SEQUENCE LISTING (388 sequences)

SEQ ID NO: 1 - DNA, 351 bp, synthetic construct
  misc_feature 1..351: note = Mus musculus GNAS 3'-UTR
  source 1..351: mol_type = other DNA
gaagggaacacccaaatttaattcagccttaagcacaattaattaagagtgaaacgtaatgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtttgtgcagcattaaaaaatcaataaaaattaaaaatgagca

SEQ ID NO: 2 - DNA, 353 bp, synthetic construct
  misc_feature 1..353: note = Mus musculus GNAS 3'-UTR
  source 1..353: mol_type = other DNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 3 - DNA, 385 bp, synthetic construct
  misc_feature 1..385: note = Homo sapiens GNAS 3'-UTR
  source 1..385: mol_type = other DNA
gaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaatgaaaaaaaaaaaaaaaaaaaa

SEQ ID NO: 4 - DNA, 1476 bp, synthetic construct
  misc_feature 1..1476: note = Homo sapiens GNAS 3'-UTR
  source 1..1476: mol_type = other DNA
tggaggacgccgtccagattctccttgttttcatggattcaggtgctggagaatctggtaaaagcaccattgtgaagcagatgaggatcctgcatgttaatgggtttaatggagagggcggcgaagaggacccgcaggctgcaaggagcaacagcgatggcagtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaatgaaaaaaaaaaaaaaaaaaaa

SEQ ID NO: 5 - DNA, 117 bp, synthetic construct
  misc_feature 1..117: note = Mus musculus MORN2 3'-UTR
  source 1..117: mol_type = other DNA
acctgctgccttaacgctgagatgtggcctctgcaaccccccttaggcaaagcaactgaaccttctgctaaagtgacctgccctcttccgtaagtccaataaagttgtcatgcaccc

SEQ ID NO: 6 - DNA, 135 bp, synthetic construct
  misc_feature 1..135: note = Mus musculus MORN2 3'-UTR
  source 1..135: mol_type = other DNA
acctgctgccttaacgctgagatgtggcctctgcaaccccccttaggcaaagcaactgaaccttctgctaaagtgacctgccctcttccgtaagtccaataaagttgtcatgcacccacaaaaaaaaaaaaaaaa

SEQ ID NO: 7 - DNA, 210 bp, synthetic construct
  misc_feature 1..210: note = Homo sapiens MORN2 3'-UTR
  source 1..210: mol_type = other DNA
catgtagatgtgatgttaaattaaagttgaaatgtagtaattgaagcttttagttgtaaggaaagcaacttaatctgttatttgaaatgacttcatacactacccctataagtttgccaataaaaccatcacctgcttacacctttttgaactttatattcattgtcttacaattagtttaaaataaatgacatgattcaaaaaaaaaaa

SEQ ID NO: 8 - DNA, 438 bp, synthetic construct
  misc_feature 1..438: note = Mus musculus GSTM1 3'-UTR
  source 1..438: mol_type = other DNA
gcccttgctacacgggcactcactaggaggacctgtccacactggggatcctgcaggccctgggtggggacagcaccctggccttctgcactgtggctcctggttctctctccttcccgctcccttctgcagcttggtcagccccatctcctcaccctcttcccagtcaagtccacacagccttcattctccccagtttctttcacatggccccttcttcattggctccctgacccaacctcacagcccgtttctgcgaactgaggtctgtcctgaactcacgcttcctagaattaccccgatggtcaacactatcttagtgctagccctccctagagttaccccgaaggtcaatacttgagtgccagcctgttcctggtggagtagcctccccaggtctgtctcgtctacaataaagtctgaaacacacttgccatg

SEQ ID NO: 9 - DNA, 455 bp, synthetic construct
  misc_feature 1..455: note = Mus musculus GSTM1 3'-UTR
  source 1..455: mol_type = other DNA
gcccttgctacacgggcactcactaggaggacctgtccacactggggatcctgcaggccctgggtggggacagcaccctggccttctgcactgtggctcctggttctctctccttcccgctcccttctgcagcttggtcagccccatctcctcaccctcttcccagtcaagtccacacagccttcattctccccagtttctttcacatggccccttcttcattggctccctgacccaacctcacagcccgtttctgcgaactgaggtctgtcctgaactcacgcttcctagaattaccccgatggtcaacactatcttagtgctagccctccctagagttaccccgaaggtcaatacttgagtgccagcctgttcctggtggagtagcctccccaggtctgtctcgtctacaataaagtctgaaacacacttgccatgaaaaaaaaaaaaaaaaa

SEQ ID NO: 10 - DNA, 531 bp, synthetic construct
  misc_feature 1..531: note = Homo sapiens GSTM1 3'-UTR
  source 1..531: mol_type = other DNA
ggccttgaaggccaggaggtgggagtgaggagcccatactcagcctgctgcccaggctgtgcagcgcagctggactctgcatcccagcacctgcctcctcgttcctttctcctgtttattcccatctttactcccaagacttcattgtccctcttcactccccctaaacccctgtcccatgcaggccctttgaagcctcagctacccactatccttcgtgaacatcccctcccatcattacccttccctgcactaaagccagcctgaccttccttcctgttagtggttgtgtctgctttaaagggcctgcctggcccctcgcctgtggagctcagccccgagctgtccccgtgttgcatgaaggagcagcattgactggtttacaggccctgctcctgcagcatggtccctgccttaggcctacctgatggaagtaaagcctcaaccacaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 133 bp, synthetic construct
  misc_feature 1..133: note = Mus musculus NDUFA1 3'-UTR
  source 1..133: mol_type = other DNA
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 12 - DNA, 148 bp, synthetic construct
  misc_feature 1..148: note = Homo sapiens NDUFA1 3'-UTR
  source 1..148: mol_type = other DNA
ggaagcattttcctgattgatgaaaaaaataactcagttatggccatctacccctgctagaaggttacagtgtattatgtagcatgcaatgtgttatgtagtgcttaataaaaataaaatgaaaaaaatgcaaaaaaaaaaaaaaaaa

SEQ ID NO: 13 - DNA, 237 bp, synthetic construct
  misc_feature 1..237: note = Mus musculus CBR2 3'-UTR
  source 1..237: mol_type = other DNA
tctgctcagttgccgcggacatctgagtggccttcttagccccaccctcagccaaagcatttactgatctcgtgactccgccctcatgctacagccacgcccaccacgcagctcacagttccacccccatgttactgtcgatcccacaaccactccaggcgcagaccttgttctctttgtccactttgttgggctcatttgcctaaataaacgggccaccgcgttacctttaactat

SEQ ID NO: 14 - DNA, 416 bp, synthetic construct
  misc_feature 1..416: note = Mus musculus YBX1 3'-UTR
  source 1..416: mol_type = other DNA
atgccggcttaccatctctaccatcatccggtttggtcatccaacaagaagaaatgaatatgaaattccagcaataagaaatgaacaaagattggagctgaagaccttaagtgcttgctttttgcccgctgaccagataacattagaactatctgcattatctatgcagcatggggtttttattatttttacctaaagatgtctctttttggtaatgacaaacgtgttttttaagaaaaaaaaaaaaggcctggtttttctcaatacacctttaacggtttttaaattgtttcatatctggtcaagttgagatttttaagaacttcatttttaatttgtaataaagtttacaacttgattttttcaaaaaagtcaacaaactgcaagcacctgttaataaaggtcttaaataataa

SEQ ID NO: 15 - DNA, 418 bp, synthetic construct
  misc_feature 1..418: note = Mus musculus YBX1 3'-UTR
  source 1..418: mol_type = other DNA
atgccggcttaccatctctaccatcatccggtttggtcatccaacaagaagaaatgaatatgaaattccagcaataagaaatgaacaaagattggagctgaagaccttaagtgcttgctttttgccctctgaccagataacattagaactatctgcattatctatgcagcatggggtttttattatttttacctaaagatgtctctttttggtaatgacaaacgtgttttttaagaaaaaaaaaaaaaaggcctggtttttctcaatacacctttaacggtttttaaattgtttcatatctggtcaagttgagatttttaagaacttcatttttaatttgtaataaagtttacaacttgattttttcaaaaaagtcaacaaactgcaagcacctgttaataaaggtcttaaataataa

SEQ ID NO: 16 - DNA, 415 bp, synthetic construct
  misc_feature 1..415: note = Homo sapiens YBX1 3'-UTR
  source 1..415: mol_type = other DNA
atgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacctaaagacgtctctttttggtaataacaaacgtgttttttaaaaaagcctggtttttctcaatacgcctttaaaggtttttaaattgtttcatatctggtcaagttgagatttttaagaacttcatttttaatttgtaataaaagtttacaacttgattttttcaaaaaagtcaacaaactgcaagcacctgttaataaaggtcttaaataataaaaaaaaaaaaaaa

SEQ ID NO: 17 - DNA, 66 bp, synthetic construct
  misc_feature 1..66: note = Mus musculus Ndufb8 3'-UTR
  source 1..66: mol_type = other DNA
ggaggcttgatgggctttttgccctcgttcctagaggcttaaccataataaaatccctaataaagc

SEQ ID NO: 18 - DNA, 125 bp, synthetic construct
  misc_feature 1..125: note = Homo sapiens Ndufb8 3'-UTR
  source 1..125: mol_type = other DNA
ggaggcttcgtgggcttttgggtcctctaactaggactccctcattcctagaaatttaaccttaatgaaatccctaataaaactcagtgctgtgttatttgtgcctcaaaaaaaaaaaaaaaaaa

SEQ ID NO: 19 - DNA, 377 bp, synthetic construct
  misc_feature 1..377: note = Homo sapiens Ndufb8 3'-UTR
  source 1..377: mol_type = other DNA
gtgaggaagaggagtgctgttcctgccttcctagcccagctgggtctgaccagaggctactgtgtacccatttaccatgcgtgattgttaactcagagtggggtgtagccaggtattgactgaatgtatgttcttgctgacctgtgtttttttctgtagggaccaaagcagtatccttacaataatctgtacctggaacgaggcggtgatccctccaaagaaccagagcgggtggttcactatgagatctgaggaggcttcgtgggcttttgggtcctctaactaggactccctcattcctagaaatttaaccttaatgaaatccctaataaaactcagtgctgtgttatttgtgcctcaaaaaaaaaaaaaaaaaa

SEQ ID NO: 20 - DNA, 922 bp, synthetic construct
  misc_feature 1..922: note = Homo sapiens CNTN1-004 3'-UTR
  source 1..922: mol_type = other DNA
tcgttgacactcaccatttctgtgaaagactttttttttttttaacatattatactagatttgactaactcaatcttgtagcttctgcagttctccccacccccaacctagttcttagagtatgtttccccttttgaaacatgtaaacatactttgggcataaatattttttaaaatataactataatgcttcactaataccttaaaaatgcctagtgaactaactcagtacattatataatggccaagtgaaagttttgtgttttcatgtcctgtttttctttgaaattatatagcccagaaattagctcattatctgaaaaacgtataagaactgatgaattgtataatacaggagtattgccattgaatgtactgtttgatttattcaagcaggtaatgaacaatgttgtcaaactctctaatgagacatcataattaggacataagctaaaaggggcattactccggcagtctttttttcttaatcctagtaccatacatattctttggcatgaaagaatgaaaagcattagtaaacaactgaagtcctaccatggctctgtagggtttttggaacaattcctggaattggaaagtgaaaatggatagcatgtgggggaaaccctcatctgagtagcaagattttagtaaagatgactaagccattaacagcatgcattcatatttaattttattgactcctgccatcagcttttgtagatcttttgggtggaaggttgtgatttttactgggaggacttgagtagaagtggatgattaaaattgaggagtatataattctttctgggactgcttaaatgttattgtttgaaaatgccttcactttccccctttggtcaaagagatgtgcttaaaattcttattccttcacaataaataattttgattttcttagaca

SEQ ID NO: 21 - DNA, 922 bp, synthetic construct
  misc_feature 1..922: note = Homo sapiens CNTN1-004 3'-UTR
  source 1..922: mol_type = other DNA
tcgttgacactcaccatttctgtgaaagacttttttttttttaacatattatactagatttgactaactcaatcttgtagcttctgcagttctccccacccccaacctagttcttagagtatgtttccccttttgaaacatgtaaacatactttgggcataaatattttttaaaatataactataatgcttcactaataccttaaaaatgcctagtgaactaactcagtacattatataatggccaagtgaaagttttgtgttttcatgtcctgtttttctttgaaattatatagcccagaaattagctcattatctgaaaaacgtatgaagaactgatgaattgtataatacaggagtattgccattgaatgtactgtttgatttattcaagcaggtaatgaacaatgttgtcaaactctctaatgagacatcataattaggacataagctaaaaggggcattactccggcagtctttttttcttaatcctagtaccatacatattctttggcatgaaagaatgaaaagcattagtaaacaactgaagtcctaccatggctctgtagggtttttggaacaattcctggaattggaaagtgaaaatggatagcatgtgggggaaaccctcatctgagtagcaagattttagtaaagatgactaagccattaacagcatgcattcatatttaattttattgactcctgccatcagcttttgtagatcgtttgggtggaaggttgtgatttttactgggaggacttgagtagaagtggatgattaaaattgaggagtatataattctttctgggactgcttaaatgttattgtttgaaaataccttcactttccccctttggtcaaagagatgtgcttaaaattcttattccttcacaataaataattttgattttcttagaca

SEQ ID NO: 22 - DNA, 928 bp, synthetic construct
  misc_feature 1..928: note = Homo sapiens CNTN1-004 3'-UTR
  source 1..928: mol_type = other DNA
ttttttcgttgacactcaccatttctgtgaaagactttttttttttttaacatattatactagatttgactaactcaatcttgtagcttctgcagttctccccacccccaacctagttcttagagtatgtttccccttttgaaacatgtaaacatactttgggcataaatattttttaaaatataactataatgcttcactaataccttaaaaatgcctagtgaactaactcagtacattatataatggccaagtgaaagttttgtgttttcatgtcctgtttttctttgaaattatatagcccagaaattagctcattatctgaaaaacgtatgaagaactgatgaattgtataatacaggagtattgccattgaatgtactgtttgatttattcaagcaggtaatgaacaatgttgtcaaactctctaatgagacatcataattaggacataagctaaaaggggcattactccggcagtctttttttcttaatcctagtaccatacatattctttggcatgaaagaatgaaaagcattagtaaacaactgaagtcctaccatggctctgtagggtttttggaacaattcctggaattggaaagtgaaaatggatagcatgtgggggaaaccctcatctgagtagcaagattttagtaaagatgactaagccattaacagcatgcattcatatttaattttattgactcctgccatcagcttttgtagatcttttgggtggaaggttgtgatttttactgggaggacttgagtagaagtggatgattaaaattgaggagtatataattctttctgggactgcttaaatgttattgtttgaaaatgccttcactttccccctttggtcaaagagatgtgcttaaaattcttattccttcacaataaataattttgattttcttagaca

SEQ ID NO: 23 - DNA, 2380 bp, synthetic construct
  misc_feature 1..2380: note = Homo sapiens CNTN1-004 3'-UTR
  source 1..2380: mol_type = other DNA
atgtgttgtgacagctgctgttcccatcccagctcagaagacacccttcaaccctgggatgaccacaattccttccaatttctgcggctccatcctaagccaaataaattatactttaacaaactattcaactgatttacaacacacatgatgactgaggcattcgggaaccccttcatccaaaagaataaacttttaaatggatataaatgatttttaactcgttccaatatgccttataaaccacttaacctgattctgtgacagttgcatgatttaacccaatgggacaagttacagtgttcaattcaatactataggctgtagagtgaaagtcaaatcaccatatacaggtgctttaaatttaataacaagttgtgaaatataatagagattgaaatgttggttgtatgtggtaaatgtaagagtaatacagtctcttgtactttcctcactgttttgggtactgcatattattgaatggcccctatcattcatgacatcttgagttttcttgaaaagacaatagagtgtaacaaatattttgtcagaaatcccattatcaaatcatgagttgaaagattttgactattgaaaaccaaattctagaacttactatcagtattcttattttcaaaggaaataattttctaaatatttgattttcagaatcagttttttaatagtaaagttaacataccatatagatttttttttacttttatattctactctgaagttattttatgcttttcttatcaatttcaaatctcaaaaatcacagctcttatctagagtatcataatattgctatatttgttcatatgtggagtgacaaattttgaaaagtagagtgcttccttttttattgagatgtgacagtctttacatggttaggaataagtgacagttaagtgaatatcacaattactagtatgttggtttttctgcttcattcctaagtattacgtttctttattgcagatgtcagatcaaaaagtcacctgtaggttgaaaaagctaccgtattccattttgtaaaaataacaataataataataataataattagttttaagctcatttcccacttcaatgcaatactgaaaactggctaaaaataccaaatcaatatactgctaatggtactttgaagagtatgcaaaactggaaggccaggaggaggcaaataatatgtctttccgatggtgtctcccaagtgttggtgctttgggtttttataagttgtgaaaaggaagatgcacatttcttcattctccatggtgtgcatggaaatgtgtttgagtgtggatgtaaaagaaatcgagtaataaagaattagctggcttgtgaaatagtgcagtgttggatgcttcaagaggtataatcctattttattagcacaaacttgctagctaattagagtttatctttttagaaaggacaccgtataggttcgtaaaaaatatttacaggaagcaaaatagatctattactactttaccgactttaccccctttctttaatttgtataatttttgtactatatatcgatgtgtaaatgtttagagtcttcattatgaaaatatcaataaatatttcattagtttacatttaactctggtataaaatgaaacttttaaaaataagtgaaatggatgatttcccagtggaagtatgtcaacagtcttaagatcattgccagatttcataaaatatttaagtatttgaaaaagaaacaaaatgtcttcatactttagggaaacgaataccctgtataccttctgtacaaatgtttgtgttttcattgttacactttggggttttacttttgcaatgtgacccatgttgggcatttttatataatcaacaactaaatcttttgccaaatgcatgcttgccttttattttctaatatatgataataacgagcaaaactggttagattttgcatgaaatggttctgaaaggtaagaggaaaacagactttggaggttgtttagttttgaatttctgacagagataaagtagtttaaaatctctcgtacactgataactcaagcttttcattttctcatacagttgtacagatttaactgggaccatcagttttaaactgttgtcaagctaactaataatcatctgctttaagacgcaagattctgaattaaactttatataggtatagatacatctgttgtttctttgtatttcaggaaaggtgatagtagttttatttgatactgataaatattgaattgattttttagttattttttatcattttttcaatggagtagtataggactgtgctttgtcctttttatgaatgaaaaaattagtataaagtaataaatgtcttatgttacccaagaaaaaa

SEQ ID NO: 24 - DNA, 1204 bp, synthetic construct
  misc_feature 1..1204: note = Homo sapiens CNTN1-004 3'-UTR
  source 1..1204: mol_type = other DNA
tcgttgacactcaccatttctgtgaaagacttttttttttttaacatattatactagatttgactaactcaatcttgtagcttctgcagttctccccacccccaacctagttcttagagtatgtttccccttttgaaacatgtaaacatactttgggcataaatattttttaaaatataactataatgcttcactaataccttaaaaatgcctagtgaactaactcagtacattatataatggccaagtgaaagttttgtgttttcatgtcctgtttttctttgaaattatatagcccagaaattagctcattatctgaaaaacgtatgaagaactgatgaattgtataatacaggagtattgccattgaatgtactgtttgatttattcaagcaggtaatgaacaatgttgtcaaactctctaatgagacatcataattaggacataagctaaaaggggcattactccggcagtctttttttcttaatcctagtaccatacatattctttggcatgaaagaatgaaaagcattagtaaacaactgaagtcctaccatggctctgtagggtttttggaacaattcctggaattggaaagtgaaaatggatagcatgtgggggaaaccctcatctgagtagcaagattttagtaaagatgactaagccattaacagcatgcattcatatttaattttattgactcctgccatcagcttttgtagatcgtttgggtggaaggttgtgatttttactgggaggacttgagtagaagtggatgattaaaattgaggagtatataattctttctgggactgcttaaatgttattgtttgaaaataccttcactttccccctttggtcaaagagatgtgcttaaaattcttattccttcacaataaataattttgattttcttagacaggtttgtgtttaggtatgagtttctcttttacttcatctagcaattctctctgtggtcagaagaactctgaagaaagctttgagggaaatgaatataactcttaaattattatatgtgtgtgtatatatatagtttaactttaaaaataatttattagtcatcataaagaaataaatgtctctggctcaagatgttacttatttccttcttttatattttctagtctcaattactgttccaaaaggagctatcttagaacttagactagagatccagattaa

SEQ ID NO: 25 - DNA, 54 bp, synthetic construct
  misc_feature 1..54: note = Mus musculus MP68 5'-UTR
  source 1..54: mol_type = other DNA
ctttcccattctgtagcagaatttggtgttgcctgtggtcttggtcccgcggag

SEQ ID NO: 26 - DNA, 97 bp, synthetic construct
  misc_feature 1..97: note = Homo sapiens MP68 5'-UTR
  source 1..97: mol_type = other DNA
cttcccggcatcccctgcgcgcgcctgcgcgctcggtgacctttccgagttggctgcagatttgtggtgcgttctgagccgtctgtcctgcgccaag

SEQ ID NO: 27 - DNA, 315 bp, synthetic construct
  misc_feature 1..315: note = Homo sapiens MP68 5'-UTR
  source 1..315: mol_type = other DNA
cttcccggcatcccctgcgcgcgcctgcgcgctcggtgacctttccgagttggctgcagatttgtggtgcgttctgagccgtctgtcctgcgccaagggagcgtaccttggccttgagaggttcagctgcctaacccagaggctacgcagagttagagaagccagagtccaagccaagaactctgactccacatccagtcccttctctcctttataactcaagtttccttgcgccacactgccctccacgttatgctgtacatgacaacttgggtgaggcaacagggaagctgaaaagagatcatacggtgctga

SEQ ID NO: 28 - DNA, 81 bp, synthetic construct
  misc_feature 1..81: note = Mus musculus NDUFA4 5'-UTR
  source 1..81: mol_type = other DNA
gtccgctcagccaggttgcagaagcggcttagcgtgtgtcctaatcttctctctgcgtgtaggtaggcctgtgccgcaaac

SEQ ID NO: 29 - DNA, 81 bp, synthetic construct; identical to SEQ ID NO: 28
  misc_feature 1..81: note = Homo sapiens NDUFA4 5'-UTR
  source 1..81: mol_type = other DNA

SEQ ID NO: 30 - DNA, 129 bp, synthetic construct
  misc_feature 1..129: note = Homo sapiens NDUFA4 5'-UTR
  source 1..129: mol_type = other DNA
gggtccttcaggtaggaggtcctgggtgactttggaagtccgtagtgtctcattgcagataatttttagcttagggcctggtggctaggtcggttctctcctttccagtcggagacctctgccgcaaac

SEQ ID NO: 31 - DNA, 186 bp, synthetic construct
  misc_feature 1..186: note = Human albumin 3'-UTR
  source 1..186: mol_type = other DNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 32 - DNA, 186 bp, synthetic construct
  misc_feature 1..186: note = albumin7 3'-UTR
  source 1..186: mol_type = other DNA
catcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 33 - DNA, 42 bp, synthetic construct
  misc_feature 1..42: note = 5'-UTR of human ribosomal protein Large 32 lacking the 5' terminal oligopyrimidine tract
  source 1..42: mol_type = other DNA
ggcgctgcctacggaggtggcagccatctccttctcggcatc

SEQ ID NO: 34 - DNA, 24 bp, synthetic construct
  misc_feature 1..24: note = histone stem-loop
  source 1..24: mol_type = other DNA
caaaggctcttttcagagccacca

SEQ ID NO: 35 - RNA, 1848 nt, synthetic construct
  misc_feature 1..1848: note = 32L4 - PpLuc(GC) - A64 - C30 - hSL
  source 1..1848: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 36 - RNA, 2201 nt, synthetic construct
  misc_feature 1..2201: note = 32L4 - PpLuc(GC) - gnas-A64-C30-hSL
  source 1..2201: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 37 - RNA, 1965 nt, synthetic construct
  misc_feature 1..1965: note = 32L4 - PpLuc(GC) - morn2- A64 - C30 - hSL
  source 1..1965: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtacctgctgccttaacgctgagatgtggcctctgcaaccccccttaggcaaagcaactgaaccttctgctaaagtgacctgccctcttccgtaagtccaataaagttgtcatgcacccagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 38 - RNA, 2286 nt, synthetic construct
  misc_feature 1..2286: note = 32L4 - PpLuc(GC) - gstm1- A64 - C30 - hSL
  source 1..2286: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgcccttgctacacgggcactcactaggaggacctgtccacactggggatcctgcaggccctgggtggggacagcaccctggccttctgcactgtggctcctggttctctctccttcccgctcccttctgcagcttggtcagccccatctcctcaccctcttcccagtcaagtccacacagccttcattctccccagtttctttcacatggccccttcttcattggctccctgacccaacctcacagcccgtttctgcgaactgaggtctgtcctgaactcacgcttcctagaattaccccgatggtcaacactatcttagtgctagccctccctagagttaccccgaaggtcaatacttgagtgccagcctgttcctggtggagtagcctccccaggtctgtctcgtctacaataaagtctgaaacacacttgccatgagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 39 - RNA, 1981 nt, synthetic construct
  misc_feature 1..1981: note = 32L4 - PpLuc(GC) - ndufa1 - A64 - C30 - hSL
  source 1..1981: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtggaagcattttcctggctgattaaaagaaattactcagctatggtcatctgttcctgttagaaggctatgcagcatattatatactatgcgcatgttatgaaatgcataataaaaaattttaaaaaatctaaaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 40 - RNA, 2085 nt, synthetic construct
  misc_feature 1..2085: note = 32L4 - PpLuc(GC) - cbr2 - A64 - C30 - hSL
  source 1..2085: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagttctgctcagttgccgcggacatctgagtggccttcttagccccaccctcagccaaagcatttactgatctcgtgactccgccctcatgctacagccacgcccaccacgcagctcacagttccacccccatgttactgtcgatcccacaaccactccaggcgcagaccttgttctctttgtccactttgttgggctcatttgcctaaataaacgggccaccgcgttacctttaactatagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 41 - RNA, 1997 nt, synthetic construct
  misc_feature 1..1997: note = PpLuc(GC) - albumin7- A64 - C30 - hSL
  source 1..1997: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 42 - RNA, 2048 nt, synthetic construct
  misc_feature 1..2048: note = Mp68 - PpLuc(GC) - albumin7- A64 - C30 - hSL
  source 1..2048: mol_type = other RNA
gggctttcccattctgtagcagaatttggtgttgcctgtggtcttggtcccgcggagaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 43 - RNA, 2075 nt, synthetic construct
  misc_feature 1..2075: note = Ndufa4 - PpLuc(GC) - albumin7- A64 - C30 - hSL
  source 1..2075: mol_type = other RNA
ggggtccgctcagccaggttgcagaagcggcttagcgtgtgtcctaatcttctctctgcgtgtaggtaggcctgtgccgcaaacaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 44 - DNA, 1810 bp, synthetic construct
  misc_feature 1..1810: note = PpLuc(GC) - A64 - C30 - hSL
  source 1..1810: mol_type = other DNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 45 - RNA, 2163 nt, synthetic construct
  misc_feature 1..2163: note = PpLuc(GC) - gnas- A64 - C30 - hSL
  source 1..2163: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 46 - RNA, 2264 nt, synthetic construct
  misc_feature 1..2264: note = 32L4 - PpLuc(GC) - Ybx1(V2)-A64-C30-hSL
  source 1..2264: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtatgccggcttaccatctctaccatcatccggtttggtcatccaacaagaagaaatgaatatgaaattccagcaataagaaatgaacaaagattggagctgaagaccttaagtgcttgctttttgcccgctgaccagataacattagaactatctgcattatctatgcagcatggggtttttattatttttacctaaagatgtctctttttggtaatgacaaacgtgttttttaagaaaaaaaaaaaaggcctggtttttctcaatacacctttaacggtttttaaattgtttcatatctggtcaagttgagatttttaagaacttcatttttaatttgtaataaagtttacaacttgattttttcaaaaaagtcaacaaactgcaagcacctgttaataaaggtcttaaataataaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 47 - RNA, 1914 nt, synthetic construct
  misc_feature 1..1914: note = 32L4 - PpLuc(GC) - Ndufb8-A64-C30-hSL
  source 1..1914: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtggaggcttgatgggctttttgccctcgttcctagaggcttaaccataataaaatccctaataaagcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 48 - RNA, 2771 nt, synthetic construct
  misc_feature 1..2771: note = 32L4 - PpLuc(GC) - Cntn1-004(V2)-A64-C30-hSL
  source 1..2771: mol_type = other RNA
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagttcgttgacactcaccatttctgtgaaagactttttttttttttaacatattatactagatttgactaactcaatcttgtagcttctgcagttctccccacccccaacctagttcttagagtatgtttccccttttgaaacatgtaaacatactttgggcataaatattttttaaaatataactataatgcttcactaataccttaaaaatgcctagtgaactaactcagtacattatataatggccaagtgaaagttttgtgttttcatgtcctgtttttctttgaaattatatagcccagaaattagctcattatctgaaaaacgtatgaagaactgatgaattgtataatacaggagtattgccattgaatgtactgtttgatttattcaagcaggtaatgaacaatgttgtcaaactctctaatgagacatcataattaggacataagctaaaaggggcattactccggcagtctttttttcttaatcctagtaccatacatattctttggcatgaaagaatgaaaagcattagtaaacaactgaagtcctaccatggctctgtagggtttttggaacaattcctggaattggaaagtgaaaatggatagcatgtgggggaaaccctcatctgagtagcaagattttagtaaagatgactaagccattaacagcatgcattcatatttaattttattgactcctgccatcagcttttgtagatcttttgggtggaaggttgtgatttttactgggaggacttgagtagaagtggatgattaaaattgaggagtatataattctttctgggactgcttaaatgttattgtttgaaaatgccttcactttccccctttggtcaaagagatgtgcttaaaattcttattccttcacaataaataattttgattttcttagacaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 49 - DNA, 201 bp, synthetic construct
  misc_feature 1..201: note = Homo sapiens SLC38A6 3'-UTR SLC38A6-001 ENST00000267488
  source 1..201: mol_type = other DNA
aagaaatattttcctacttcttacaagaataatatacccctagttgcaagaatgaattattccggaagacaccctggatgaaaaataacattttaataaaaattattaacagaaaagcagaacaaaatggcagtgggtatggggaagtaagagtgtggcagttttaatcaaaaaaagaaacaaactcgaaatgctcttaaa

SEQ ID NO: 50 - DNA, 102 bp, synthetic construct
  misc_feature 1..102: note = Homo sapiens DECR1 3'-UTR NM_001359.1
  source 1..102: mol_type = other DNA
gaccactttggccttcatcttggttacagaaaagggaatagaaatgaaacaaattatctctcatcttttgactatttcaagtctaataaattcttaattaac

SEQ ID NO: 51 - DNA, 369 bp, synthetic construct
  misc_feature 1..369: note = Homo sapiens PIGK 3'-UTR
  source 1..369: mol_type = other DNA
acttgatgatgaatgaagaatgcatggaggactgcaaacttggataataatttatgtcattatatatttttaaaaatgtgtttctcttgtatgaattggaaataagtataaggaaactaaatttgaatcaactattaattttataacttaaagaaaaataattgttaatgcaactgcttaatggcactaaatatattccagttttgtattttgtgtattataaaagcgaatgagacagagatcagaatacattgactgtttttgaaaatagtaatttccccttatccccttttcatttggaaaagaaacaattgtgaagacattaaattctcactaacagaagtaactttggttaattattttttgtat

SEQ ID NO: 52 - DNA, 460 bp, synthetic construct
  misc_feature 1..460: note = Homo sapiens FAM175A 3'-UTR FAM175A-009 ENST00000506553
  source 1..460: mol_type = other DNA
tccttttaaccttacaaggagatttttttatttggctgatgggtaaagccaaacatttctattgtttttactatgttgagctacttgcagtaagttcatttgtttttactatgttcacctgtttgcagtaatacacagataactcttagtgcatttacttcacaaagtactttttcaaacatcagatgcttttatttccaaacctttttttcacctttcactaagttgttgaggggaaggcttacacagacacattctttagaattggaaaagtgagaccaggcacagtggctcacacctgtaatcccagcacttagggaagacaagtcaggaggattgattgaagttaggagttagagaccagcctgggcaacgtattgagaccatgtctattaaaaaataaaatggaaaagcaagaatagccttattttcaaaatatggaaagaaatttatatgaaaa

SEQ ID NO: 53 - DNA, 505 bp, synthetic construct
  misc_feature 1..505: note = Homo sapiens PHYH 3'-UTR PHYH-002 ENST00000396913
  source 1..505: mol_type = other DNA
aatagccatctgctataactctttcaacagaaaaccaaaaccaaacgaaatgtctaaggaaaatgttttcttaatgagatgatgtaaccttttctatcacttgttaaaagcagaaaacatgtatcaggtacttaattgcatagagttagttttgcagcacaatggtgttgctttaatggaaaaaaaaaacagtaaaagtgaaatattactgttttaaggaaaactaatttagggtggcagccaataaaggtggttggtgtctaatttaagtgttaaatcaatttctttcattcagttagctctttacccaagaagaagtgaatgatttggagcttagggtatgttttgtatcccctttctgataaacccattccctaccaattttatgtcataagagatttttttcccccaaatctagaacaatgtataatacattcacatctagtcaagggcataggaacggtgtcatggagtccaaataaagtggatattcctgctcggacaa

SEQ ID NO: 54 - DNA, 404 bp, synthetic construct
  misc_feature 1..404: note = Homo sapiens TBC1D19 3'-UTR TBC1D19-001 ENST00000264866
  source 1..404: mol_type = other DNA
tcttcttcacagtcactggcaacacatctagtttttcattagaaacaaatcatgaactatgcaaactctgcataaaaccaaaatgaaactttgcatataagccaataaagatcatgttccctcttcagttaaacctaagtagtttctcactttttgaaacaataactctgcaccaaatattgcatcgcatgctgctgattttcaagagagaagcaataaacacaacttctgctaaattgagcattatatatataatattataatatatatataatcctgacttgtcaatggcatgtaataatatatgcaataagaactaaagatactgtaataaacttcaagaggtaatgtagcttcttggataattcttttatgtcagtttataaatttatctctagataatg

SEQ ID NO: 55 - DNA, 353 bp, synthetic construct
  misc_feature 1..353: note = Homo sapiens TBC1D19 NM_018317.2 3'-UTR
  source 1..353: mol_type = other DNA
tcttcttcacagtcactggcaacacatctagtttttcattagaaacaaatcatgaactatgcaaactctgcataaaaccaaaatgaaactttgcatataagccaataaagatcatgttccctcttcagttaaacctaagtagtttctcactttttgaaacaataactctgcaccaaatattgcatcgcatgctgctgattttcaagagagaagcaataaacacaacttctgctaaattgagcattatatatataatattataatatatatataatcctgacttgtcaatggcatgtaataatatatgcaataagaactaaagatactgtaataaacttcaagaggtaaaaaaa

SEQ ID NO: 56 - DNA, 242 bp, synthetic construct
  misc_feature 1..242: note = Homo sapiens PIGB 3'-UTR PIGB-201 ENST00000539642
  source 1..242: mol_type = other DNA
aaattcaacatgaagatgaaattctgaactttcctagataaattaacattgctgggtggaaatattcagatgctgcttaaatacttcggtaaacactgggtaagattcatggaacttagaaaaaagctgtatgaactgctttaccaaatatcactactgaggaaatgtataaaataccacatagtataaaattacatgttaatacaatgccagattttaaataaagacctttagttttcctc

SEQ ID NO: 57 - DNA, 157 bp, synthetic construct
  misc_feature 1..157: note = Homo sapiens ALG6 3'-UTR ALG6-006 ENST00000263440
  source 1..157: mol_type = other DNA
ctgtattcctaaacaaattgtttcctaaacaaatgtgaaaatgtgaacagtgctgaaaggttttgtgaactttttgctatgtataaatgaaattaccattttgagaaccatggaaccacaggaaaggaaatggtgaaaagtcattgttgtctacaca

SEQ ID NO: 58 - DNA, 324 bp, synthetic construct
  misc_feature 1..324: note = Homo sapiens CRYZ 3'-UTR CRYZ-005 ENST00000370871
  source 1..324: mol_type = other DNA
tgattaattctttcatggatttcctatgtaattagaggtactgtctttcccccagttgtacttaccctatcttttctttaattaacattcgattccatgagcttcttatgtgaaaaaataagatttttctttagagagcagaagcagaagagtaaaatttattgtatagctagcaatatttttttatgccatctgtctcaaatcaaagagtcatcatagtaggaaataacatgttagttgtcatttggcatgagtgtgcattccagtaattcttaattgatatttgattaattccatacctttgattaaaacatgctagttcaa

SEQ ID NO: 59 - DNA, 510 bp, synthetic construct
  misc_feature 1..510: note = Homo sapiens BRP44L 3'-UTR BRP44L-001 ENST00000360961
  source 1..510: mol_type = other DNA
caatggaaaaggaagaacaaggtcttgaagggacagcattgccagctgctgctgagtcacagatttcattataaatagcctccctaaggaaaatacactgaatgctatttttactaaccattctatttttatagaaatagctgagagtttctaaaccaactctctgctgccttacaagtattaaatattttacttctttccataaagagtagctcaaaatatgcaattaatttaataatttctgatgatggttttatctgcagtaatatgtatatcatctattagaatttacttaatgaaaaactgaagagaacaaaatttgtaaccactagcacttaagtactcctgattcttaacattgtctttaatgaccacaagacaaccaacagctggccacgtacttaaaattttgtccccactgtttaaaaatgttacctgtgtatttccatgcagtgtatatattgagatgctgtaacttaatggcaataaatgatttaaatatttgttaaa

SEQ ID NO: 60 - DNA, 354 bp, synthetic construct
  misc_feature 1..354: note = Homo sapiens ACADSB 3'-UTR ACADSB-004
  source 1..354: mol_type = other DNA
cgtctataggagtgggacccctccctggtgtcactgctgtaaaattttaaacggttgtgtcttgttgggagtaagtgccttgcgtgggaataaacttccacagcattcgaatattttaatgaagcccttagtcagggtcctggtgttggcctttttggttttctcttttcaggctgtttaacttaggcacaggagatccacttttaaacttgggaaataagcacctgtatttttttccaaaactgtttttaaagctgtatacgcatacatatatatatttttactctgtcttactctgtcacccaggctagagtgcagtggcgcgatctcagctcactgcagccttgacctcct

SEQ ID NO: 61 - DNA, 539 bp, synthetic construct
  misc_feature 1..539: note = Homo sapiens TMEM14A 3'-UTR NM_014051.3
  source 1..539: mol_type = other DNA
gcatctggaggaacagaaaactaagttcatgtcatcctgctgtaatgggcagagcatattttttttgtatttaaaagataaacttcaatatggaatgctagaaacacaaatagcactgtcacctctaatatgaacattagtttgaggtagtttttttctaaagcaaaaattttaactgttttctaattgtcaagcactattttcattaaaagtgtctaatgaatcatgatatactcttccatttgttgtgtctattttttatatatttggtattttttgaaaattccaaatactcatgtctcaagtaagcttaaactacaacttgtcacataaaggaagtcttaagtggagttcacagaatgataatgtatctatttgtcatttgtgttatatttgaaattattagaaattatgctttttccattttaattgtattgctgccagtgctatttttttctttaaaaaattttattcttagcacactgttatgtcctaactgaatgtattcagtattcaaataaaagacattttggttcaaa

SEQ ID NO: 62 - DNA, 292 bp, synthetic construct
  misc_feature 1..292: note = Homo sapiens GRAMD1C 3'-UTR GRAMD1C-005 ENST00000472026
  source 1..292: mol_type = other DNA
tgatctgaaggactaaaaccgcagagatacttggaacttaaagaaaatacctggaagaaaaccagacgaatgaaggattttggcatagaacatttctatgttttttcattattgagatttctaatatgaacatttctttcagtaacatttatttgataattagtttctgctggccttaataatccatcctttcacttcttatagatatttttaagctgtgaatttcttcagtgaaccatgaaatatattatagaactgaatttctctgatacaaaaagaaaatgacacaccc

SEQ ID NO: 63 - DNA, 94 bp, synthetic construct
  misc_feature 1..94: note = Homo sapiens C11orf80 3'-UTR C11orf80-201ENST00000360962
  source 1..94: mol_type = other DNA
gccgggtccccttccgcaagcgcccaccgatccggaggctgcgggcagccgttatcccgtggtttaataaagctgccgcgcgctcaccaagtcc

SEQ ID NO: 64 - DNA, 266 bp, synthetic construct
  misc_feature 1..266: note = Homo sapiens ANXA4 3'-UTR ANXA4-002 ENST00000409920
  source 1..266: mol_type = other DNA
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 65 - DNA, 490 bp, synthetic construct
  misc_feature 1..490: note = Homo sapiens TBCK 3'-UTR TBCK-002 ENST00000361687
  source 1..490: mol_type = other DNA
agaaccaagagtgtgactgccaaaacttagtgtggcatcagcaccaacagcacagttcttcatatccacgccactctcagacaaaactagatgtccagattgttgcatttccgtaaagtttgtcacgagacattttttaaaatctcataacccacatgttcagttatccatgcaagaaacttgactctacatgtattgctgaaagaattttcttaacagtgaaatctgatcatatatttttaccacactgccacataaagcccaagaaattcagctgacaagacagatttagcattatcaagaaatcccatttgccctgaaaaagctgtcctccattgtactgaacagacagtcctgtcgattgtgttatttagaaacatacactgaatgtgggctgaaatcatcatctttccataatgaaaactgagaaactattcacaatgcattccttataaataaatgctacatttagtaactcatttcacccaaa

SEQ ID NO: 66 - DNA, 320 bp, synthetic construct
  misc_feature 1..320: note = Homo sapiens IFI6 3'-UTR IFI6-001 ENST00000361157
  source 1..320: mol_type = other DNA
ccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 67 - DNA, 479 bp, synthetic construct
  misc_feature 1..479: note = Homo sapiens CAMKMT 3'-UTR (synonym C2orf34) ENST00000378494
  source 1..479: mol_type = other DNA
aagattaagcttctcaaagacgaagaaacgtatcaagtgcatagggaatatttttacaaaaacggaaatctgtaaggggtataatcgcctgcctgcgccctttgcagcatttcacgtgtgggctatggactccacctgtcctcacccacgttattccccagctgccctctccagctccctccccgcctctttttacactctgcttgttgctcgtcctgccctaaacctttgtttgtctttaaatgtgtataagctgcctgtctgtgacttgaatttgactggtgaacaaactaaatatttttccctgtaattgagacagaatttcttttgatgatacccatccctccttcattttttttttttttttggtctttgttctgttttggtggtggtagtttttaatcagtaaacccagcaaatatcatgattctttcctggttagaaaaataaataaagtgtatctttttatctccctccaa

SEQ ID NO: 68 - DNA, 476 bp, synthetic construct
  misc_feature 1..476: note = Homo sapiens ALDH6A1 3'-UTR NM_005589.2
  source 1..476: mol_type = other DNA
aaacaagtttgtttaagactgactccatcctgagtaatctccctttatttttgaccagcttcatttgtcagctttgctcagatcagatcgatgggattggaatacattgtaactaaaatcttcctcaggactattaacccccgcaaagtttctatagggaactgcctagtgtaacaatgaaaccagatttctcacttgctcttcatacttctattttgaggtaactgttgtaactatgaaatgcttatctgaaagtagtgcttaaacctgatttctaaaaattatcccattttctgatgatttgaaggggagaaaagccagtgtatgtaaagaaaatgttccagccaggcgcggtggctcacgcctgtaattccatcattttgggaggccacagtgggcagattgcttgagcccaggagttgaagaacgtggcgaaaccccgtatctattatttaaaaaaattgaaaaagtaaaaa

SEQ ID NO: 69 - DNA, 567 bp, synthetic construct
  misc_feature 1..567: note = Homo sapiens AGTPBP1 3'-UTR AGTPBP1-004 ENST00000357081
  source 1..567: mol_type = other DNA
gcccgctgccatctcttgttaactgcaaagaataaatgaaatatcttggtttttatttcccaggaagcttgagagaaatgagtttatacagagctgactcaaaaagacaaaaagtaacttgggccagtttggtttcaagataataaatgtgttattaattaatgataaaattggcgcttgttttattttcgatattcaatgcactttatgtagcattgaatgatcaaatattggatttacctttaaaaaaaaaacctgagtatcattgcatgaatttttatctccctatggttatatcctgcatcaagtggataattttgaagtgtgttcagaatataaaattgaaattttagagttgttgaaaatcctgacttgttgaaaactaatatatatgtacatggatttctatagatgtgtttgtttagaagtgggtagatattgcagataagactgttcttcagaatcatgttaactattgggttgtgactgaagtagtccagggtttgccttgaaaccattacattctacatttaccaaattaaacaaataaaaactgtattaaatgtt

SEQ ID NO: 70 - DNA, 169 bp, synthetic construct
  misc_feature 1..169: note = Homo sapiens CCDC53 3'-UTR CCDC53-001 ENST00000240079
  source 1..169: mol_type = other DNA
gcttaattttgataagaattacatatgcatgcataggggtacatttacattctgtaagagattgagcctgaactctcttagtcataaaaacatcaaatggccacatgtccactaccaagcttcttctatgttaaaaaaataataataaagcagttttaacctgccagta

SEQ ID NO: 71 - DNA, 194 bp, synthetic construct
  misc_feature 1..194: note = Homo sapiens LRRC28 3'-UTR LRRC28-002 ENST00000331450
  source 1..194: mol_type = other DNA
taaacactcaagaacctcaggagcgctgccagcttgacactggggaatccagccagtccagcacactcttccatcctgtcctgtccaatgcgggggcactgcagaactctctagaaatgtcatgattgagcttcagagctaaaatgccttcacccttcccccaagttggaatatatcctcccccaaattaagga

SEQ ID NO: 72 - DNA, 120 bp, synthetic construct
  misc_feature 1..120: note = Homo sapiens CCDC109B 3'-UTR NM_017918.4
  source 1..120: mol_type = other DNA
tcttacagttttaaatgtcgtcagattttccattatgtattgattttgcaacttaggatgtttttgagtcccatggttcattttgattgtttaatctttgttattaaattcttgtaaaac

SEQ ID NO: 73 - DNA, 426 bp, synthetic construct
  misc_feature 1..426: note = Homo sapiens PUS10 3'-UTR PUS10-001 ENST00000316752
  source 1..426: mol_type = other DNA
ctttcaaatttggagacaaagagtatggttttcctggcatgatgtggacatccatggagcacatgccgtaaaatggctgtttacccaccataacggtgtcttgaaaactatttggatcatgttgatctatataattgttaatttgttgtaacatctcaggatctatatatgtgtatattttgtgttaaattgttccaaggatgtcttaggatttttctcattccctctttcacccccacaaaccaaactatgaataatgaaataattctccttaattctttcatttagagaggtgcacaaacaggacacattctctgttaacctaagaagctgtaatttcagcaagatttccctccacaagagatataccacctttaaaatcatgttctaatttttgtaaattatctgaataaaagttatatctag

SEQ ID NO: 74 - DNA, 71 bp, synthetic construct
  misc_feature 1..71: note = Homo sapiens CCDC104 3'-UTR CCDC104-002 ENST00000339012
  source 1..71: mol_type = other DNA
taattaagaacaatttaacaaaatggaagttcaaattgtcttaaaaataaattatttagtccttacactga

SEQ ID NO: 75 - DNA, 125 bp, synthetic construct
  misc_feature 1..125: note = Homo sapiens CASP1 3'-UTR CASP1-007 ENST00000527979
  source 1..125: mol_type = other DNA
aataaggaaactgtatgaatgtctgtgggcaggaagtgaagagatccttctgtaaaggtttttggaattatgtctgctgaataataaacttttttgaaataataaatctggtagaaaaatgaaaa

SEQ ID NO: 76 - DNA, 174 bp, synthetic construct
  misc_feature 1..174: note = Homo sapiens SNX14 3'-UTR SNX14-007 ENST00000513865
  source 1..174: mol_type = other DNA
acacttggatttggtatagaataacccattgaaatttctgctgtgcgagggtggtagaaatttacttttttgggtatattcttatatatattatgtacatcgctgtctgaaattttagttattttttgtttttaataaagactaacacaaacttaatgattaaaagtgattgag

SEQ ID NO: 77 - DNA, 237 bp, synthetic construct
  misc_feature 1..237: note = Homo sapiens SKAP2 3'-UTR SKAP2-201 (part of SKAP2.001 ENST00000345317)
  source 1..237: mol_type = other DNA
gagtcctggaaaaggaaaattcttctgcttgtctgcaaatgctttggatttagaagcgtcatgaaagcacgagtgacagctcctaacctctccttgttttattaaacattacttatctttgactgttattttatgcagtcgctcattaaaatattcctctgatgtgaaattaaatgaaggatattaatgtaaattagatgcaaccagttaagttatacctgttgctattttgcaaag

SEQ ID NO: 78 - DNA, 362 bp, synthetic construct
  misc_feature 1..362: note = Homo sapiens NDUFB6 3'-UTR NM_182739.2
  source 1..362: mol_type = other DNA
agattatgtaaaaagttaaaaggcttatgagcctaagtttgttcctatattaccatatttactgaattttctggaaaagtaactttaataaagtttaatctcagaaattgtcatatctgttttcaagcattgtacaatttgagactgagtaatttaacaataagtaaaaagtggacatgctaaacaaatatgagagactacctactttttctggtcattcttgacttggaaaacggtatggaaaagtatttagttacatgtttgtttgtttttttcttacacagtacttacactaatttggtatcagggtatgcaacagtgaaatatcacaataaacaaatgtaagaacaaaaaaaaaaaaa

SEQ ID NO: 79 - DNA, 549 bp, synthetic construct
  misc_feature 1..549: note = Homo sapiens EFHA1 3'-UTR EFHA1-001 ENST00000382374
  source 1..549: mol_type = other DNA
taaaagatataatagtatggcaattatattgttccaaatgtcaaaatttgtgattttttagaagtacttgctatttatcttcttaagtcttcattgatattctgtgtgaaataagcatgtcttgtacttgctttctgattcataattttattaaagaacttagtagaaagaaaagtaagtataaaaatagatattggattctgtcagaaggcctagatttgaaataatgttttgtacttcggtaagatggaaaacttagtgattcactgatttcttagacactctaatatgatatgctttctggaaggataaaacaaatacatatgggaaaaagtacttgagaccaaggccagcatcaattccagacatcttcatgttcctaataggctaaatgaagttaaaaacttatttcagatttttctcatctgtaccttatatctcataaatttattgcatattttatgtcagtagcttagctgtttattgtctttaaaataacatgtaaacttcaatgttctatctggaagcagaataaaatatttacataga

SEQ ID NO: 80 - DNA, 288 bp, synthetic construct
  misc_feature 1..288: note = Homo sapiens BCKDHB 3'-UTR BCKDHB-005 ENST00000356489
  source 1..288: mol_type = other DNA
ccatatagaaaagctggaagattatgactagatatggaaatattttttctgaatttttttttatatttcctccgacttacctctttttgaaaagagagtttttattaagtgaaccatcacgatattggctgaaaagttctacattctattattgtattgtaacacacatgtattgatgattttcattaagagtttcagattaactttgaaaaatattccacatggtaatcttataaattctgtttaattacatctgtaaatattatgtgtgtgatagtattcaataaa

SEQ ID NO: 81 - DNA, 414 bp, synthetic construct
  misc_feature 1..414: note = Homo sapiens BCKDHB 3'-UTR NM_001164783.1
  source 1..414: mol_type = other DNA
gacctgctcagcccacccccacccatcctcagctaccccgagaggtagccccactctaaggggagcagggggacctgacagcacaccactgtcttccccagtcagctccctctaaaatactcagcggccagggcggctgccactcttcacccctgctcctcccggctgttacattgtcaggggacagcatctgcagcagttgctgaggctccgtcagccccctcttcacctgttgttacagtgccttctcccaggggctgggtgagggcacattcaggactagaagcccctctgggcatggggtggacatggcaggtcagcctgtggaacttgcgcaggtgcgagtggccagcagaggtcacgaataaactgcatctctgcgcctggctctctaccaaaaaaaaaaaaaaaaaa

SEQ ID NO: 82 - DNA, 414 bp, synthetic construct
  misc_feature 1..414: note = Homo sapiens BBS2 3'-UTR NM_031885.3
  source 1..414: mol_type = other DNA
gtgaggaaaatacaggtcatgaagttcctggcaaagattttctgttaaaaacctatgctggtttgctttggatcacaccctggtgaaccccgggtgctaagaatgaaaataaccttggtgagttgtacaaattaaagacaaagaactacatgtgaagatagacttgctttctatttttaaatcagtagtagtactgttgctgaataatactaggtttttatggaataggatgaatgcttttgaagtattagggcttcagagtccaattttgcttatttatggtatataaatacatatttttttcttgaaattgcaattgagtttgtacttttcaaatagattatctactttttcattaaaatgtaaagatgttaaactttgtgttgattgattataaaatcaccaccaaatcag

SEQ ID NO: 83 - DNA, 409 bp, synthetic construct
  misc_feature 1..409: note = Homo sapiens LMBRD1 3'UTR NM_018368.3
  source 1..409: mol_type = other DNA
cagccttctgtcttaaaggttttataatgctgactgaatatctgttatgcatttttaaagtattaaactaacattaggatttgctaactagctttcatcaaaaatgggagcatggctataagacaactatattttattatatgttttctgaagtaacattgtatcatagattaacattttaaattaccataatcatgctatgtaaatataagactactggctttgtgagggaatgtttgtgcaaaattttttcctctaatgtataatagtgttaaattgattaaaaatcttccagaattaatattcccttttgtcactttttgaaaacataataaatcatctgtatctgtgccttaggttctccagagtgatgtggaattttaaagtgtctctctctgattgcctccaa

SEQ ID NO: 84 - DNA, 466 bp, synthetic construct
  misc_feature 1..466: note = Homo sapiens ITGA6 3'-UTR ITGA6-003 ENST00000409532
  source 1..466: mol_type = other DNA
tattgatctacttctgtaattgtgtggattctttaaacgctctaggtacgatgacagtgttccccgataccatgctgtaaggatccggaaagaagagcgagagatcaaagatgaaaagtatattgataaccttgaaaaaaaacagtggatcacaaagtggaacgaaaatgaaagctactcatagcgggggcctaaaaaaaaaaagcttcacagtacccaaactgctttttccaactcagaaattcaatttggatttaaaagcctgctcaatccctgaggactgatttcagagtgactacacacagtacgaacctacagttttaactgtggatattgttacgtagcctaaggctcctgttttgcacagccaaatttaaaactgttggaatggatttttctttaactgccgtaatttaactttctgggttgcctttatttttggcgtggctgacttacatcatgtgtt

SEQ ID NO: 85 - DNA, 285 bp, synthetic construct
  misc_feature 1..285: note = Homo sapiens HERC5 3'-UTR HERC5-001 ENST00000264350
  source 1..285: mol_type = other DNA
ccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgggaaatatttttcaaaaataaaataaccgaaaaattaa

SEQ ID NO: 86 - DNA, 515 bp, synthetic construct
  misc_feature 1..515: note = Homo sapiens HADHB 3'-UTR HADHB-001 ENST00000317799
  source 1..515: mol_type = other DNA
tagatccagaagaagtgacctgaagtttctgtgcaacactcacactaggcaatgccatttcaatgcattactaaatgacatttgtagttcctagctcctcttaggaaaacagttcttgtggccttctattaaatagtttgcacttaagccttgccagtgttctgagcttttcaataatcagtttactgctctttcagggatttctaagccaccagaatctcacatgagatgtgtgggtggttgtttttggtctctgttgtcactaaagactaaatgagggtttgcagttgggaaagaggtcaactgagatttggaaatcatctttgtaatatttgcaaattatacttgttcttatctgtgtcctaaagatgtgttctctataaaatacaaaccaacgtgcctaattaattatggaaaaataattcagaatctaaacaccactgaaaacttataaaaaatgtttagatacataaatatggtggtcagcgttaataaagtggagaaatattggagaa

SEQ ID NO: 87 - DNA, 116 bp, synthetic construct
  misc_feature 1..116: note = Homo sapiens ANAPC4 3'-UTR ANAPC4-001 ENST00000315368
  source 1..116: mol_type = other DNA
tctagcttgccattattgtgtgtgtaattatggccaaaaggacataggagatggactaagatgtcttggaccacctttgtgtaacaaagaaataaacagtaaattttattttttca

SEQ ID NO: 88 - DNA, 154 bp, synthetic construct
  misc_feature 1..154: note = Homo sapiens PCCB 3'-UTR NM_000532.4
  source 1..154: mol_type = other DNA
acaaatcaaaggaaaagaaaccaagaactgaattactgtctgcccattcacatcccattcctgccttttgcaatcatgaaacctgggaatccaaatagttggataacttagaataactaagtttattaaattctagaaagatctcaaaaaaaaa

SEQ ID NO: 89 - DNA, 90 bp, synthetic construct
  misc_feature 1..90: note = Homo sapiens ABCB7 3'-UTR ABCB7-001 ENST00000253577
  source 1..90: mol_type = other DNA
gtcacataagacattttctttttttgttgttttggactacatatttgcactgaagcagaattgttttattaaaaaaatcatacattccca

SEQ ID NO: 90 - DNA, 318 bp, synthetic construct
  misc_feature 1..318: note = Homo sapiens PGCP 3'-UTR CPQ-001 ENST00000220763
  source 1..318: mol_type = other DNA
aaacagtaagaaagaaacgttttcatgcttctggccaggaatcctgggtctgcaactttggaaaactcctcttcacataacaatttcatccaattcatcttcaaagcacaactctatttcatgctttctgttattatctttcttgatactttccaaattctctgattctagaaaaaggaatcattctcccctccctcccaccacatagaatcaacatatggtagggattacagtgggggcatttctttatatcacctcttaaaaacattgtttccactttaaaagtaaacacttaataaatttttggaagatctctga

SEQ ID NO: 91 - DNA, 133 bp, synthetic construct
  misc_feature 1..133: note = Homo sapiens NFU1 3'-UTR NM_001002755.2
  source 1..133: mol_type = other DNA
aataatctggattttctttgggcataacagtcagacttgttgataatatatatcaagtttttattattaatatgctgaggaacttgaagattaataaaatatgctcttcagagaatgatatataaatattgca

SEQ ID NO: 92 - DNA, 246 bp, synthetic construct
  misc_feature 1..246: note = Homo sapiens OMA1 3'-UTR OMA1-001 ENST00000371226
  source 1..246: mol_type = other DNA
attaaaatttatgagacacaagatatatgaagaatgttgcagtccttatcattttatgttactttttaaaaaatgatgtttgaagtgaaaaaaaaaaggatattcagggtcaaatcatgtacattacagatattatctaaattcttctagaatttatttttcatgaaatattgatgtattttaatctatgttaaaatatcttcaatgaggaaaatgtcacagaataaatttatattacacatttta

SEQ ID NO: 93 - DNA, 423 bp, synthetic construct
  misc_feature 1..423: note = Homo sapiens HHLA3 3'-UTR NM_001036646.1
  source 1..423: mol_type = other DNA
ggcgaatccatagagtaagcttagtgatgtgtgtcagacctctgagcccaagcaaagccatcatatcccctgtgacctgcatgtatacatccagatggcctgaagcaagtgaagaatcacaaaagaagtgaaaagggccggttcctgccttaactgatgacattccaccattgtgatttgttcctgccccaccttaactgagcgattaacctgtgaacttccttctcctggctcagaagcttccccactgagcaccttgtgacccccgcccctgcctgccatagaacaaccccctttgattgtaattttcctttacctacccaaatcctataaaacggccccacccctatctcccttcgctgacactctctttggactcagcctgcctgcacctaggtgattaaaaagctttattgctcacgc

SEQ ID NO: 94 - DNA, 292 bp, synthetic construct
  misc_feature 1..292: note = Homo sapiens HHLA3 3'-UTR NM_001031693.2
  source 1..292: mol_type = other DNA
aaagggccggttcctgccttaactgatgacattccaccattgtgatttgttcctgccccaccttaactgagcgattaacctgtgaacttccttctcctggctcagaagcttccccactgagcaccttgtgacccccgcccctgcctgccatagaacaaccccctttgattgtaattttcctttacctacccaaatcctataaaacggccccacccctatctcccttcgctgacactctctttggactcagcctgcctgcacctaggtgattaaaaagctttattgctcacgc

SEQ ID NO: 95 - DNA, 342 bp, synthetic construct
  misc_feature 1..342: note = Homo sapiens ACAA2 3'-UTR NM_006111.2
  source 1..342: mol_type = other DNA
agagaccagtgagctcactgtgacccatccttactctacttggccaggccacagtaaaacaagtgaccttcagagcagctgccacaactggccatgccctgccattgaaacagtgattaagtttgatcaagccatggtgacacaaaaatgcattgatcatgaataggagcccatgctagaagtacattctctcagatttgaaccagtgaaatatgatgtatttctgagctaaaactcaactatagaagacattaaaagaaatcgtattcttgccaagtaaccaccacttctgccttagataatatgattataaggaaatcaaataaatgttgccttaacttc

SEQ ID NO: 96 - DNA, 446 bp, synthetic construct
  misc_feature 1..446: note = Homo sapiens GSTM4 3'-UTR GSTM4-001 ENST00000369836
  source 1..446: mol_type = other DNA
tgccttgaaggccaggaggtgggagtgaggagcccatactcagcctgctgcccaggctgtgcagcgcagctggactctgcatcccagcacctgcctcctcgttcctttctcctgtttattcccatctttacccccaagactttattgggcctcttcacttcccctaaacccctgtcccatgcaggccctttgaagcctcagctacccactttccttcatgaacatccccctcccaacactacccttccctgcactaaagccagcctgaccttccttcctgttagtggttgtatctgctttgaagggcctacctggcccctcgcctgtggagctcagccctgagctgtccccgtgttgcatgacagcattgactggtttacaggccctgctcctgcagcatggcccctgccttaggcctacctgatcaaaataaagcctcagccaca

SEQ ID NO: 97 - DNA, 465 bp, synthetic construct
  misc_feature 1..465: note = Homo sapiens GSTM4 3'-UTR GSTM4-003 ENST00000326729
  source 1..465: mol_type = other DNA
tggtcaattttctgcatcaacttgactgggctaagggatgctcagatggcaggtaaaatcattgtgcttgtgagggtgtttccagaagagatttgcctttgaatcagaagacagcaaagatttccttcagcaatgaaggaggcatccaccaaactgtcagggcccagagagaagaaaaagacaggaagggtgaatttgacctctctgactgggacatccatctctgcctatcctgggacctccacactcctggttctctggccttcagacttgatcagggactaacaccatcgcctcccacccccacctttgttctgaggcctttagcctctgaatgataccactggctttcctgcttctctatcctgcagtcggcagatcatgggacttcttcactccaaaattgtgtgagccaattcccataacagatagataaatttataaataaacacacaaatttcctac

SEQ ID NO: 98 - DNA, 274 bp, synthetic construct
  misc_feature 1..274: note = Homo sapiens ALG8 3'-UTR NM_001007027.2
  source 1..274: mol_type = other DNA
ctgaaacctccgcctcccagaaaagaaaaacctctttttaattggatggaaactttctacctgcttggcctggggcctctggaagtctgctgtgaatttgtattccctttcacctcctggaaggtgaagtaccccttcatccctttgttactaacctcagtgtattgtgcagtaggcatcacatatgcttggttcaaactgtatgtttcagtattgattgactctgctattggcaagacaaagaaacaatgaataaaggaactgcttagatatg

SEQ ID NO: 99 - DNA, 122 bp, synthetic construct
  misc_feature 1..122: note = Homo sapiens C11orf74 3'UTR
  source 1..122: mol_type = other DNA
ttcacagaggcattttgtgtgtgtgtgcttattttaattttgttcttattctagcaacattagaataaaagataaacctactataattccctttgtggaaatttaaaaaaaaaaaaaaaaaa

SEQ ID NO: 100 - DNA, 133 bp, synthetic construct; identical to SEQ ID NO: 11
  misc_feature 1..133: note = Mus musculus Ndufa1 3'-UTR Ndufa1-001 ENSMUST00000016571
  source 1..133: mol_type = other DNA

SEQ ID NO: 101 - DNA, 155 bp, synthetic construct
  misc_feature 1..155: note = Mus musculus Atp5e 3'-UTR NM_025983
  source 1..155: mol_type = other DNA
ctgaatctgaagcctgaagtgctgagtcttgaaggtgaagcatgtgggcccctgttctggcagatggaaatcaacctcacctcctgggggacaggctgcccatctcgttgataaattgactatgccaataaattaacatggttcactttcaaaaa

SEQ ID NO: 102 - DNA, 136 bp, synthetic construct
  misc_feature 1..136: note = Mus musculus Gstm5 3'-UTR NM_010360
  source 1..136: mol_type = other DNA
gccagagctcgctgctgctgagccatcttgccctgaggggcccacactcttagctcactgtcagtcttgttccatcctgtcctgagggcccccactctgtctcctctgctctttctaataaacagcagttgcatta

SEQ ID NO: 103 - DNA, 189 bp, synthetic construct
  misc_feature 1..189: note = Mus musculus Uqcr11 3'-UTR NM_025650
  source 1..189: mol_type = other DNA
gcagcccctcccccaccacaggcctcgatggtaccatgtgccgaggcctcagacacagcgtagtcctgtggaagacactgaggaagctggacactggagaggtctgcaccgctcagggagcttccatgttgacagacactagggctgccttgatgggtgcagcattaaaccttattcttatgccttgga

SEQ ID NO: 104 - DNA, 143 bp, synthetic construct
  misc_feature 1..143: note = Mus musculus IFi27I2a 3'-UTR IFi27I2a-001 ENSMUST00000055071; NM_029803
  source 1..143: mol_type = other DNA
gcttaggagatgacacttctatcagctcaactcaaagcctgtacagactacgcaggagatgaagttccaaaaggcaccttcagaaccctcactgatgtcaaagaatgatgaaaacaacaaagtatatgggctggtgttcctaa

SEQ ID NO: 105 - DNA, 237 bp, synthetic construct; identical to SEQ ID NO: 13
  misc_feature 1..237: note = Mus musculus Cbr2 3'-UTR NM_007621
  source 1..237: mol_type = other DNA

SEQ ID NO: 106 - DNA, 118 bp, synthetic construct
  misc_feature 1..118: note = Mus musculus Atp5l 3'-UTR Atp5l-201 ENSMUST00000043675
  source 1..118: mol_type = other DNA
agaccaatctttaacttctgatttgagttcttatttgaatgttcttggaccatgtgtaacaggactgctatctgaataaaatactaggtgttgaaaacactgctgtgttttctctgtc

SEQ ID NO: 107 - DNA, 271 bp, synthetic construct
  misc_feature 1..271: note = Mus musculus Tmsb10 3'-UTR NM_025284
  source 1..271: mol_type = other DNA
aagcctaggaagatttccccaccccaccccaccccgccccatcatctccaagaccccctcgtgatgtggaggaagagccacctgcaagatggacgcgagccacaagctgcactgtgaaacccgggcactccgagccgatgccaccggcccgcgggtctctgaaggggacccctccactaatcggactgccaaatttcaccggtttgccctgggatattatagaaaattatttgtatgattgatgaaaataaaaacacctcgtggcatggtt

SEQ ID NO: 108 - DNA, 116 bp, synthetic construct
  misc_feature 1..116: note = Mus musculus Nenf 3'-UTR NM_025424
  source 1..116: mol_type = other DNA
tgtctagctgagaagcagccggttctagggagaagtgaggggacaggagttaagtgtccctcggaacaagcggaggaagcctccgagtgccctgcagctgaataaagcgaatgttt

SEQ ID NO: 109 - DNA, 123 bp, synthetic construct
  misc_feature 1..123: note = Mus musculus Atp5k 3'-UTR NM_007507
  source 1..123: mol_type = other DNA
ggcgtcagcgagcttgcttttctctagtcgttgagaacgaataaagcttcattgtgtgaaaaaaaaaaaaaaaaaaaaaaaaaaaaaaaaaaaaaaaaaaaaaaaaaaaaaaaaaaaaaaaaa

SEQ ID NO: 110 - DNA, 260 bp, synthetic construct
  misc_feature 1..260: note = Mus musculus 1110008P14Rik 3'-UTR 1110008P14Rik-001 ENSMUST00000048792
  source 1..260: mol_type = other DNA
gtgccgggagcccccatccaggccctaccctcacctctctaggccatgttctggcctgggtagatactacttggcttagacaccatctcgggtactggcctccagatcctagtgggtctaccagcctggaccagtccccattcactgcccatcacccttcctggagtcaggtgcaatcctacagttctcccacttgtctgtcttctttcccctccatccagactgagagtccgaattaaagatgtctcccacaccactgc

SEQ ID NO: 111 - DNA, 102 bp, synthetic construct
  misc_feature 1..102: note = Mus musculus Cox4i1 3'-UTR NM_009941
  source 1..102: mol_type = other DNA
gagcccgctgcctgccggctccctgcctccctcactccctcggcatgctggaagctgccgtatccaatggtccatgctaataaaagaccagtttacgtggtg

SEQ ID NO: 112 - DNA, 189 bp, synthetic construct
  misc_feature 1..189: note = Mus musculus Cox6a1 3'-UTR NM_007748
  source 1..189: mol_type = other DNA
agagaacctggcctcccccaggcaacaaagggaccacagcactggttttggacccttactctgtgtggaccacgaaaaccctttggatgctaagctcgtgtctcctttcctcagatggcgaccattactctgatcttccatcccttctgcttgtaagaggagatgccttaaataaataacttaaactca

SEQ ID NO: 113 - DNA, 139 bp, synthetic construct
  misc_feature 1..139: note = Mus musculus Ndufs6 3'-UTR NM_010888
  source 1..139: mol_type = other DNA
tgtgggctgtgtcctggtcctctgactcctatggaacatctccacgctgggtgttctgtgtgaggccactgctctgtgaatggtgtcccttgttttgaataaaggatgctcccaccatgaaaaaaaaaaaaaaaaaaaa

SEQ ID NO: 114 - DNA, 171 bp, synthetic construct
  misc_feature 1..171: note = Mus musculus Sec61b 3'-UTR NM_024171
  source 1..171: mol_type = other DNA
attgggctacatccatctgtcatctgaagaagaagaagaaggaaaaaaacccaacatatcttggaccaaaagtgtagtgattttctgttcacgtgtattattttacagagaataagaattgactttgagaaatcagttttttctatggctaataaactttggaattgcttt

SEQ ID NO: 115 - DNA, 101 bp, synthetic construct
  misc_feature 1..101: note = Mus musculus Romo1 3'-UTR NM_025946
  source 1..101: mol_type = other DNA
ttagggctaggatgccctgcaatacctaaacttccccatccatttcgacccttgtacaataataaagttgttttcttctcgttaaaaaaaaaaaaaaaaaa

SEQ ID NO: 116 - DNA, 370 bp, synthetic construct
  misc_feature 1..370: note = Mus musculus Gnas 3'-UTR NM_010309
  source 1..370: mol_type = other DNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagcaaaaaaaaaaaaaaaaaa

SEQ ID NO: 117 - DNA, 96 bp, synthetic construct
  misc_feature 1..96: note = Mus musculus Snrpd2 3'-UTR NM_026943
  source 1..96: mol_type = other DNA
agcctgctccctgccctgcgaaggcctgcagaaccctgcccagtgggcgagaaataaaaccctgtgctttttggttaaaaaaaaaaaaaaaaaaaa

SEQ ID NO: 118 - DNA, 119 bp, synthetic construct
  misc_feature 1..119: note = Mus musculus Mgst3 3'-UTR NM_025569
  source 1..119: mol_type = other DNA
ggtgtggagggccttccgactctcactcacctccagcgactcaccctgatttccagttgcactggttttttttttttttttaatataataaaaacttatctggcatcagcctcatacct

SEQ ID NO: 119 - DNA, 304 bp, synthetic construct
  misc_feature 1..304: note = Mus musculus Aldh2 3'-UTR NM_009656
  source 1..304: mol_type = other DNA
agcggcatgcctgcttcctcagcccgcacccgaaaacccaacaagatatactgagaaaaaccgccacacacactgcgcctccaaagagaaaccccttcaccaaagtgtcttgggtcaagaaagaattttataaacagggcggggctggtgggggggaaagctcctgataaactgggtaggggatgaagctcaatgcagaccgatcacgcgtccagatgtgcaggatgctgccttcaacctgcagtccctaagcagcaaatgagcaataaaaatcagcagatcaaagccacggggtcagttctct

SEQ ID NO: 120 - DNA, 134 bp, synthetic construct
  misc_feature 1..134: note = Mus musculus Mp68 (2010107E04Rik) 3'-UTR NM_027360
  source 1..134: mol_type = other DNA
ctgctccgaatccacaagatgaagacgtcggctaaacttgagcaagctttgttagatgggaacatggaacatcactgtacacttatctaagtaccatttataatgtggcattaataaatgtatctgtgaatacc

SEQ ID NO: 121 - DNA, 87 bp, synthetic construct
  misc_feature 1..87: note = Mus musculus Ssr4 3'-UTR NM_001166480
  source 1..87: mol_type = other DNA
gggcagcaacttcagccgtccattgcttctttcaataaacagtcactatttgacatgagtacattcaagaaaaaaaaaaaaaaaaaa

SEQ ID NO: 122 - DNA, 186 bp, synthetic construct
  misc_feature 1..186: note = Mus musculus Myl6 3'-UTR NM_010860
  source 1..186: mol_type = other DNA
ggacattctgtatcccgagtctgttccttgcccagtgtgatttctgtgtggctccagaggctcccctgtcacagcaccttgcccatttggtttcttttggatgatgtttgccttccccaaataaaatttgctctctttgccctccaaaaaaaaaaaaaaaaaaaaaaaaaaaaaaaaaaaaaaaaa

SEQ ID NO: 123 - DNA, 58 bp, synthetic construct
  misc_feature 1..58: note = Mus musculus Prdx4 3'-UTR Prdx4-001, NM_016764
  source 1..58: mol_type = other DNA
aaagtacttcagttatgatgtttggaccttctcaataaaggtcattgtgttattacca

SEQ ID NO: 124 - DNA, 130 bp, synthetic construct
  misc_feature 1..130: note = Mus musculus Ubl5 3'-UTR NM_025401
  source 1..130: mol_type = other DNA
agggggattccttctcctcctcgccctgctctgccctgccctcctctcccatcctcatctgacactggtgtagatggtcatttttaacagttcacatgaataaaaacttggctgctgctttgctgctgtc

SEQ ID NO: 125 - DNA, 87 bp, synthetic construct
  misc_feature 1..87: note = Mus musculus 1110001J03Rik 3'-UTR NM_025363
  source 1..87: mol_type = other DNA
tgcagagagtcctcagatgttccttcattcaagagtttaaccatttctaacaatatgtagttatcattaaatcttttttaaagtgtg

SEQ ID NO: 126 - DNA, 33 bp, synthetic construct
  misc_feature 1..33: note = Mus musculus Ndufa13 3'-UTR Ndufa13-201 ENSMUST00000110167
  source 1..33: mol_type = other DNA
ggcctgagccaacgcacataataaagagtggtc

SEQ ID NO: 127 - DNA, 69 bp, synthetic construct
  misc_feature 1..69: note = Mus musculus Ndufa3 3'-UTR NM_025348
  source 1..69: mol_type = other DNA
atgcctctgctgatggaagaggccccttccctgttgctctccaataaaaatgtgaaaactaataacccc

SEQ ID NO: 128 - DNA, 96 bp, synthetic construct
  misc_feature 1..96: note = Mus musculus Gstp2 3'-UTR NM_181796
  source 1..96: mol_type = other DNA
tggactgaagagacaagagcttcttgtccccgttttcccagcactaataaagtttgtaagacaaaaaaaaaaaaaaaaaaaaaaaaaaaaaaaaaa

SEQ ID NO: 129 - DNA, 89 bp, synthetic construct
  misc_feature 1..89: note = Mus musculus Tmem160 3'-UTR NM_026938
  source 1..89: mol_type = other DNA
acaacagggctgtggggactggctgggcctgacgactgggacattaaaacctgacccttccgcaaaaaaaaaaaaaaaaaaaaaaaaaa

SEQ ID NO: 130 - DNA, 129 bp, synthetic construct
  misc_feature 1..129: note = Mus musculus Ergic3 3'-UTR NM_025516
  source 1..129: mol_type = other DNA
ctctctcccttccccacagcttgtcctgccctctcttcccctgtgggtttaccctccagcctgtcaactacccatatcctctcctcagccagcccagcccagggcaataaatatgaattgtgataggaa

SEQ ID NO: 131 - DNA, 295 bp, synthetic construct
  misc_feature 1..295: note = Mus musculus Pgcp 3'-UTR NM_018755
  source 1..295: mol_type = other DNA
ggagaacaagaagagaggaccttgttctctgtagttgggaatcccaactctgaatctttacaacatccatcgtcacaaaagagtgttatacatttaatccacagggcatagttttctttataccttctgttaatcatctttccttaatactttcttatctgtttctagaataaatcatgatccctactgcaccaccttgaaaatgttgtttccagttttaaaataagcaataaatatttgaaatgcttctgatttttcattttcatttaaaaacattaaattaaatgtaatgaga

SEQ ID NO: 132 - DNA, 263 bp, synthetic construct
  misc_feature 1..263: note = Mus musculus Slpi 3'-UTR NM_011414
  source 1..263: mol_type = other DNA
gcctgatccctgacattggcgccggctctggactcgtgctcggtgtgctctggaaactacttccctgctcccaggcgtccctgctccgggttccatggctcccggctccctgtatcccaggcttggatcctgtggaccagggttactgttttaccactaacatctccttttggctcagcattcaccgatctttagggaaatgctgttggagagcaaataaataaacgcattcatttctctatgcaaaaaaaaaaaaaaaaaaa

SEQ ID NO: 133 - DNA, 241 bp, synthetic construct
  misc_feature 1..241: note = Mus musculus Myeov2 3'-UTR NM_001163425
  source 1..241: mol_type = other DNA
ggccgcccggtcctatgtgctccatgtctgtgatgtgtctggagtctctcgggacacgaccagctgattgtagacaccgtgttgatatcactagaaatgaagaccttgtcaaccaatagaggaactgtctgaaccaactgggtactgatgtctctgggaatgccagcccgtgtccttgtttaagttaataaagaacactgtaacacgcagggtgattttaaaaaaaaaaaaaaaaaaaaaa

SEQ ID NO: 134 - DNA, 162 bp, synthetic construct
  misc_feature 1..162: note = Mus musculus Ndufa4 3'-UTR NM_010886
  source 1..162: mol_type = other DNA
actatgaagttcactgtaaagctgctgataatgaaggtctttcagaagccatccgcacaattttccacttaagcaggaaatatgtctctgaatgcatgaaatcatgttgattttttttttttttggagtttattacactgatgaataaatctctgaaacttg

SEQ ID NO: 135 - DNA, 143 bp, synthetic construct
  misc_feature 1..143: note = Mus musculus Ndufs5 3'-UTR NM_001030274
  source 1..143: mol_type = other DNA
gcggggcagctggaggccgctgtcatgctctgttttcccctggagagaatatttaaggaaagctccttcattaagtattaagtatgtggaaataaagaattactcagtcttaaaaaaaaaaaaaaaaaaaaaaaaaaaaaaaa

SEQ ID NO: 136 - DNA, 455 bp, synthetic construct; identical to SEQ ID NO: 9
  misc_feature 1..455: note = Mus musculus Gstm1 3'-UTR NM_010358
  source 1..455: mol_type = other DNA

SEQ ID NO: 137 - DNA, 109 bp, synthetic construct
  misc_feature 1..109: note = Mus musculus 1810027O10Rik 3'-UTR 1810027O10Rik-001 ENSMUST00000094065
  source 1..109: mol_type = other DNA
agtctcttgtttaagcgcccagtcctggcctttctgggtaattgggcgcagagggaaggagccaatgttgaagcagaaaagaaattaaaagaaaaaggcatataaagaa

SEQ ID NO: 138 - DNA, 49 bp, synthetic construct
  misc_feature 1..49: note = Mus musculus 1810027O10Rik 3'-UTR BC117077
  source 1..49: mol_type = other DNA
agtctcttgtttaagcgcccagtcctggcctttctgggtaattgggcgc

SEQ ID NO: 139 - DNA, 55 bp, synthetic construct
  misc_feature 1..55: note = Mus musculus Atp5o 3'-UTR NM_138597
  source 1..55: mol_type = other DNA
gagactgtcacctgtgtgagctcttgtccttggagcaacaataaaatgcttcctg

SEQ ID NO: 140 - DNA, 174 bp, synthetic construct
  misc_feature 1..174: note = Mus musculus Shfm1 3'-UTR NM_009169
  source 1..174: mol_type = other DNA
catctgggaatgtcccaggaacctcaatcatggactctaccacagtctaggacagagaaagcaggacgggatactttaaagaacatgtttatttcattatctgcttcaatttatttttgttttataacaaaaaaaataagtaaataaatgttttgatttaatctttttggttca

SEQ ID NO: 141 - DNA, 260 bp, synthetic construct
  misc_feature 1..260: note = Mus musculus Tspo 3'-UTR NM_009775
  source 1..260: mol_type = other DNA
aggcacccagccatcaggaatgcagccctgccagccaggcaccatgggtggcagccatcatgcttttatgaccattgggcctgctggtctacctggtcttagcccaggaagccaccaggtaggttagggtggtcagtgccgagtctcctgcagacacagttatacctgcctttctgcactgctccaggcatgcccttagagcatggtgttttaaagctaaataaagtctctaacttcatgtgtaaaaaaaaaaaaaaaaa

SEQ ID NO: 142 - DNA, 92 bp, synthetic construct
  misc_feature 1..92: note = Mus musculus S100a6 3'-UTR NM_011313
  source 1..92: mol_type = other DNA
aatgggaccgttgagatgacttccgggggcctctctcggtcaaatccagtggtgggtagttatacaataaatatttcgtttttgttatgcct

SEQ ID NO: 143 - DNA, 200 bp, synthetic construct
  misc_feature 1..200: note = Mus musculus Taldo1 3'-UTR NM_011528
  source 1..200: mol_type = other DNA
tgcaacacccgaggccccagtcctgcaccgaggctgaccccagacctgcactgcctttgagctgggtcctaattgcacatggcttgtgacgaatgaatcttgcattttttagtgatcggagaagggatggatcataggattctgattttatgtgaaattttgtctaattcattaaagcagttgcttttcctatgctgttt

SEQ ID NO: 144 - DNA, 83 bp, synthetic construct
  misc_feature 1..83: note = Mus musculus Bloc1s1 3'-UTR NM_015740
  source 1..83: mol_type = other DNA
actaaaacccacccctcttacttcaccctcctggacaggagggaaactggtgagccacgaataaaaacacaagcttccattct

SEQ ID NO: 145 - DNA, 93 bp, synthetic construct
  misc_feature 1..93: note = Mus musculus Ndufb11 3'-UTR NM_019435
  source 1..93: mol_type = other DNA
tggcttaccgagcagggcctaagaagcattactcatccgctgcttgttatttacctggttcctcagaacaccttattaaaggaattgaaagta

SEQ ID NO: 146 - DNA, 454 bp, synthetic construct
  misc_feature 1..454: note = Mus musculus Map1lc3a 3'-UTR NM_025735
  source 1..454: mol_type = other DNA
gtcaagaggaggggaggggggtggctgggagttctggtcaggttctccccagggaggtcctggctcctaaactaagctatttcagtccccagtggattaggcagagatgtgacacccactcccccccccaggtaggggccaccagccagcctaccacatcctgggtaggtcctgggccagtcatgttcgggttgctcttttgggtgctggctgggttgggagtgggtggggagcagcatccctgctctgtggggtttgtcattttgttaggcccttgcctgtctgcccatcttgcccctcatccacctgaggctttgcctcctgccaggacctgccccacccctgaaaggctggctccccttgtcctgactcggtgtatggatctgtggtcatttcctctgcagaaagaataaagactgctcaggcctgcctggccaaaaaaaaaaaaaaaaaa

SEQ ID NO: 147 - DNA, 135 bp, synthetic construct; identical to SEQ ID NO: 6
  misc_feature 1..135: note = Mus musculus Morn2 3'-UTR NM_194269
  source 1..135: mol_type = other DNA

SEQ ID NO: 148 - DNA, 238 bp, synthetic construct
  misc_feature 1..238: note = Mus musculus Gpx4 3'-UTR NM_008162.2
  source 1..238: mol_type = other DNA
ctagccctacaagtgtgtgcccctacaccgagcccccctgccctgtgacccctggagccttccaccccggcactcatgaaggtctgcctgaaaaccagcctgctggtggggcagtcctgaggacctggcgtgcatccctgccggaggaaggtccagaggcctgtggccctgggctcgagcttcaccctggctgccttgtgggaataaaatgtagaaatgtgaaaaaaaaaaaaaaaaa

SEQ ID NO: 149 - DNA, 124 bp, synthetic construct
  misc_feature 1..124: note = Mus musculus Mif 3'-UTR NM_010798.2
  source 1..124: mol_type = other DNA
gtcctggccccacttacctgcaccgctgttctttgagcctcgctccacgtagtgttctgtgtttatccaccggtagcgatgcccaccttccagccgggagaaataaatggtttataagagacca

SEQ ID NO: 150 - DNA, 135 bp, synthetic construct
  misc_feature 1..135: note = Mus musculus Cox6b1 3'-UTR NM_025628
  source 1..135: mol_type = other DNA
cctggctccgcccacctctcctctgttctttgtctttctccccggatagaaaagggggacctcagcatatgatggtccttaccctgggaccctgaatcatgatgcaactactaataaaaactcactggaaaagtt

SEQ ID NO: 151 - DNA, 267 bp, synthetic construct
  misc_feature 1..267: note = Mus musculus RIKEN cDNA2900010J23 (Swi5) 3'-UTR NM_175190
  source 1..267: mol_type = other DNA
gcagcttcttggagattttcatctacagcccacagggacaggaggatgggggcataaaaggcagagtctagacagtatgttcatatggttttcagattttaaaagatgctagaagccctccaaagtttggggtgggttctagagaagaggagtattgggaggggtgggtattgtcaatgttaaggttcctaaacatacttgtgagtaggtgtgtgtggttgtcccttttgttaataaacatatgagcagtcaaaaaaaaaaaaaaaa

SEQ ID NO: 152 - DNA, 160 bp, synthetic construct
  misc_feature 1..160: note = Mus musculus Sec61g 3'-UTR NM_011343.3
  source 1..160: mol_type = other DNA
gtccttctcatcatgggacgagtgagccagagcgggggaaagggcatgaagtaaagcgttgcctgaatgctgtgtggtgttttgtttcttcctccttcctatgaggttttctacttctcaattaaaataatttcaaaataaacactttttccataacaga

SEQ ID NO: 153 - DNA, 62 bp, synthetic construct
  misc_feature 1..62: note = Mus musculus 2900010M23Rik 3'-UTR BC_030629
  source 1..62: mol_type = other DNA
ccgtggggtctgatactcatcaataaaactgcctggtttctcccacaaaaaaaaaaaaaaaa

SEQ ID NO: 154 - DNA, 338 bp, synthetic construct
  misc_feature 1..338: note = Mus musculus Anapc5 3'-UTR Anapc5-201 ENSMUST00000086216
  source 1..338: mol_type = other DNA
ccaggactccctgcttgatggtgtgcatttaggggtgggtcattacatgctatcttgtcaataaactgttctgatcagtttgtctgaagtgggtttttttttatttttctgggttgaattgtcagtatctttgttaagaactgtgtatctaggggctggagagatggcttagcagttaagagcactaactgttcttctaaaggacctgggttcaattcctagcaccctcatgacagctcacagctgtctgtaactcctgttccagggactctgacaccctcaggcagacataaaagcagtcaaaacaccgatgtacataaaattaaaataaattattt

SEQ ID NO: 155 - DNA, 71 bp, synthetic construct
  misc_feature 1..71: note = Mus musculus Mars2 3'-UTR BC132343.1
  source 1..71: mol_type = other DNA
gaactcagctcttactgactggtagtaaaagatcaaatgtattctttttgcgtttttaagtaaagtcatgc

SEQ ID NO: 156 - DNA, 176 bp, synthetic construct
  misc_feature 1..176: note = Mus musculus Phpt1 3'-UTR NM_029293
  source 1..176: mol_type = other DNA
agctctgccccaccccccaccccccggactaagtcaggtctctgctcttgctgtgttctgttttgaggggctggccctgtgctttccttttgtaccttaggcagcatagcacctgccaggccttagaggccagaccaatctggtccataggaattaaaagcattgatatgcctact

SEQ ID NO: 157 - DNA, 66 bp, synthetic construct; identical to SEQ ID NO: 17
  misc_feature 1..66: note = Mus musculus Ndufb8 3'-UTR NM_026061
  source 1..66: mol_type = other DNA

SEQ ID NO: 158 - DNA, 170 bp, synthetic construct
  misc_feature 1..170: note = Mus musculus Pfdn5 3'-UTR NM_027044
  source 1..170: mol_type = other DNA
gagtgcactgcagaaatgaagcagagtgagggacccttcttcaaggggcctgggactttttccggcaatggcctcctgggaaagtggcctgggaagagagtgttttgtgtttaatgttaataaatgtgaccgctgcgcaaaaaaaaaaaaaaaaaaaaaaaaaaaaaaaa

SEQ ID NO: 159 - DNA, 278 bp, synthetic construct
  misc_feature 1..278: note = Mus musculus Arpc3 3'-UTR NM_019824
  source 1..278: mol_type = other DNA
gaggagcctgggcagcaccatcacgtggagacacatcataggacacacaggccaatgtgtctgttcatacctaccgtatcaaggagagaagagagcctgtctttgctggaaaagctcttggtcaagaattgggagggtgggtgttgggcgatttcgatttttggcagttttaagctggtacttaatatataataaatgtcactgcttatgttagacattgaattaaaacatttttgagaaaaagctttaaaaaaaaaaaaaaaaaaaaaaaaaaaaaa

SEQ ID NO: 160 - DNA, 58 bp, synthetic construct
  misc_feature 1..58: note = Mus musculus Ndufb7 3'-UTR NM_025843
  source 1..58: mol_type = other DNA
ggattacccgccagcctgtggacctatcagtgaaataaaagctttgggtcacctgcct

SEQ ID NO: 161 - DNA, 51 bp, synthetic construct
  misc_feature 1..51: note = Mus musculus Atp5h 3'-UTR NM_027862
  source 1..51: mol_type = other DNA
agcagcctgggacggagccccggccgacatgaaataaaacatttaaatagt

SEQ ID NO: 162 - DNA, 80 bp, synthetic construct
  misc_feature 1..80: note = Mus musculus Mrpl23 3'-UTR NM_011288
  source 1..80: mol_type = other DNA
cctatgacagcaggatttggaccacagaccctagtgagcacagtggttctgacaagcccaaataaaaattctttgtggag

SEQ ID NO: 163 - DNA, 387 bp, synthetic construct
  misc_feature 1..387: note = Mus musculus Tomm6 3'-UTR NM_025365.3
  source 1..387: mol_type = other DNA
ccagagaatggaactcctgtgtattcagactttccaaagacagcctactgtctgtgaccacaagatcctacctgagtggcagctgaagttgactccctctccttgcctgaacccccccccactgcccccccatcccccagtgtcggctgagatgttgcctctgcacggttctgtgtgcagttcccaactttctgcagaagatggtccttgcccttgtcctgaagagtagtaatggttcttgaaaaagatttcaaataaagcctgcacataaaagacaggtattttattcttttaataagaaacttattacaaaaacaaggtgtaaaaagtccgcttacaaaaatcaaataaacatgacttgtatttcaaaaaaaaaaaaaaaaaaaa

SEQ ID NO: 164 - DNA, 327 bp, synthetic construct
  misc_feature 1..327: note = Mus musculus Tomm6 3'-UTR Tomm6-002 ENSMUST00000113301
  source 1..327: mol_type = other DNA
ccaggtgagagcagttctcctgtgtttccccgtttctgatgctgttatctgcttacagagaatggaactcctgtgtattcagactttccaaagacagcctactgtctgtgaccacaagatcctacctgagtggcagctgaagttgactccctctccttgcctgaacccccccccactgcccccccatcccccagtgtcggctgagatgttgcctctgcacggttctgtgtgcagttcccaactttctgcagaagatggtccttgcccttgtcctgaagagtagtaatggttcttgaaaaagatttcaaataaagcctgcacataaaa

SEQ ID NO: 165 - DNA, 273 bp, synthetic construct
  misc_feature 1..273: note = Mus musculus Tomm6 3'-UTR
  source 1..273: mol_type = other DNA
ccagagaatggaactcctgtgtattcagactttccaaagacagcctactgtctgtgaccacaagatcctacctgagtggcagctgaagttgactccctctccttgcctgaacccccccccactgcccccccatcccccagtgtcggctgagatgttgcctctgcacggttctgtgtgcagttcccaactttctgcagaagatggtccttgcccttgtcctgaagagtagtaatggttcttgaaaaagatttcaaataaagcctgcacataaaa

SEQ ID NO: 166 - DNA, 631 bp, synthetic construct
  misc_feature 1..631: note = Mus musculus Mtch1 3'-UTR NM_019880
  source 1..631: mol_type = other DNA
cctaagctgcccgaccaaacatttatggggtcttagcctacccctggtgaggacccatcatctcagatgcccaagggtgactccagcccagcctggcttcatgtccatatttgccatgtgtctgtccagatgtgggctggtggaggtgggtcacctgggacctggggaagcctgggggagcagtgttggggtggcatccccttcctgcctagaggtactggagtccatcttgtactcaggcagaggcaggctgcagaggcaaacgtcactcagtggcaaggcttccctgcacctctagcccagctcatcctgccagtcagccagaagcacccccgccccccacttcctgctttgtaaattgggcgccatcacacctgggccatgggaggctggagctatgttcccaacactaattttcttatacaagggtggtgccttctcctgaataggaaatcatgttctcctcagaccatcccctcatctgcttgtctgtgctggtgacgccaggtgtgagggttcagtcactgtgctgggtgcgaatacgcacaggttacataggccgacatctagtcctcccctcgtggtaagatagacccatctcctcgaataaatgtattggtggtgatttgga

SEQ ID NO: 167 - DNA, 158 bp, synthetic construct
  misc_feature 1..158: note = Mus musculus Pcbd2 3'-UTR NM_028281
  source 1..158: mol_type = other DNA
tctgcgcctgccttgtctgcagcgttgtttgcaagccacttatgttaataaattgtcataaagtagttcatagttacatgtatacattgttgtatgattgatgctcaaatacagaatgatttgaagccaaaaaaaaaaaaaaaaaaaaaaaaaaaaaa

SEQ ID NO: 168 - DNA, 93 bp, synthetic construct
  misc_feature 1..93: note = Mus musculus Ecm1 3'-UTR NM_007899
  source 1..93: mol_type = other DNA
gtcaccctgagcctcagaggattagatgggggaactccgccctactccaccctcctcgaacactcattacaataaatgcctcttggatttggc

SEQ ID NO: 169 - DNA, 458 bp, synthetic construct
  misc_feature 1..458: note = Mus musculus Hrsp12 3'-UTR Hrsp12-001 ENSMUST00000022946
  source 1..458: mol_type = other DNA
ctataagtagccatgctgatgttgactccggaggttttagaatgtctttcacactttaatttttacaaatgatgctgggaagtataaaaatgaccagagtggttgaagttattgtggaagtgatcaaatatgtggagatttgacattaattggagattattcagtatagtgactgatgttctaatttcacttatgttgctgggtgtgagagaagaggtgcacagctactgagatgggaagcagaaggaaagatgggctgttgtacatgagaaatagtaaggagcacatctacttaaatcatattaatttgctcatgtgaaatacttagttcttatgttagatataagaaactaaattgaaatattcaaacttgaatagtaccaggagaacaagtggaccaaaatcttatacagataatattactttaattgaaataaaaaatagatgtgtaactttcc

SEQ ID NO: 170 - DNA, 183 bp, synthetic construct
  misc_feature 1..183: note = Mus musculus Mecr 3'-UTR NM_025297
  source 1..183: mol_type = other DNA
ttgctccagaggaccaggaggaaagcaggagaggcaagactggctgtctgctggcccctccatgagaaccccagccttcccagactgcctcacccatattgtctcttcctaccaggagggtgggggaccaactctaggctccctaataaacccttaacttcccgagtggaggatgaagagtac

SEQ ID NO: 171 - DNA, 120 bp, synthetic construct
  misc_feature 1..120: note = Mus musculus Uqcrq 3'-UTR NM_025352
  source 1..120: mol_type = other DNA
acggcctgcacctgggtgacagtcccctgcctctgaaagacccttctctgggagaggaatccacactgtagtcttgaagacaataaactacttatggacttccctttgaaaaaaaaaaaa

SEQ ID NO: 172 - DNA, 511 bp, synthetic construct
  misc_feature 1..511: note = Mus musculus Gstm3 3'-UTR NM_010359
  source 1..511: mol_type = other DNA
gcccctgccatgctgtcactcagagtgggggacctgtccatactgcggatcctgcaggctctgggtggggacagcaccctggccttctgcactgtggctcccggttctctctccttcccgctcccttctgcagcttggtcagccccatctcctcatcctcaccccagtcaagcccatgcagcctttattctccccatttttttttcacatggccccttcttcattggtgcccagacccaacctcacagcccttttctgcaatctgaggtctgtcctgaactcaggctccctagagttaccccaatggtcaacactatcttagtgccagccctccctagagataccctgatggtcaatactatcttagtgacggccctccctagagttaccctgaaggtcaatactcgagtgccagcctgttcctgtttaaggagctgccccaggcctgtctcatgtacaataaagcctgaaacacacttgaaacacaataaacactgaacacttgctgtga

SEQ ID NO: 173 - DNA, 312 bp, synthetic construct
  misc_feature 1..312: note = Mus musculus Lsm4 3'-UTR NM_015816
  source 1..312: mol_type = other DNA
tcactccctgcctgagccgagcccagaacggtgggtgaggcctcagggcacctttgtgtgaagccccacttggcgtctggtccagtgaagtccctcgctggccactgactcagtttctggaaggttccgagtctgaggtgcctgtggagccttagatgccctttgaagggctgacttcttccaggcatgtttgagtttcagttggagctgcaggctcagcccatggcggctcacctgtcctttaccagccataccctgtacatcttctgtttgaaaaataaaagcaaacaccatagaaagaaaaaaaaaaaa

SEQ ID NO: 174 - DNA, 195 bp, synthetic construct
  misc_feature 1..195: note = Mus musculus Park7 3'-UTR NM_020569
  source 1..195: mol_type = other DNA
agcccaagccctgggccccacgcttgagcaggcattggaagcccactggtgtgtccagagcccagggaacctcagcagtagtatgtgaagcagccgccacacggggctctcatcccgggtctgtatgtttctgaaccttgctagtagaataaacagtttaccaagctcctgccagctaaaaaaaaaaaaaaaaaa

SEQ ID NO: 175 - DNA, 131 bp, synthetic construct
  misc_feature 1..131: note = Mus musculus Usmg5 3'-UTR NM_023211
  source 1..131: mol_type = other DNA
atggattttgaaatgtctgacctcacctgttaagtcccatgcctgaagaagctgatgtgaactcatcatgtaatactcaatttgtacaataaattatgaacccaaaaaaaaaaaaaaaaaaaaaaaaaaaa

SEQ ID NO: 176 - DNA, 231 bp, synthetic construct
  misc_feature 1..231: note = Mus musculus Cox8a 3'-UTR NM_007750
  source 1..231: mol_type = other DNA
agggagcagtcttccctcatcctttgactagaccacttttgccagcccaccttgatcatgttgcctgcattcctggctggccttccccgggatcatgttattcaattccagtcacctcttctgcaatcatgacctctcgatgtctccatggtgacaactgggaccacatgtattggctctgcttggtggggtccccctttgtaacaataaagtctatttaaaccttgctcc

SEQ ID NO: 177 - DNA, 403 bp, synthetic construct
  misc_feature 1..403: note = Mus musculus Ly6c1 3'-UTR NM_010741
  source 1..403: mol_type = other DNA
tggtccttccaatgacccccacccttttccttttatcttcatgtgcaaccactctttcctggagtcctctagtgacaaattatatgttatagaaggtccaatgtggggatagtgtgtggaacaccctgtttcacctttatagcccctgctgggtaagtgcccgactcctctctagggctttcaaatctgtacttcttgcaatgccatttagttgtggatttctattcttggccctggaggcatgtggccagcacatgcaacaggcagtattccaaggtattatagtatcaccatccacacataagtatctggggtcctgcagggttcccatgtatgcctgtcaatgacccctgttgagtccaataaaagctttgttctcccagccaaaaaaaaaaaaaaaaaa

SEQ ID NO: 178 - DNA, 309 bp, synthetic construct
  misc_feature 1..309: note = Mus musculus Ly6c1 3'-UTR NM_001252058.1
  source 1..309: mol_type = other DNA
actcataaaaatgctcctgcctcggtcttccaagttctaggattgcaagtctgacttcaacatgccttacagacaactctgggacatccaggcctagtggcatgttgcccagatatggggatgctctgtggcccctgcataagaagtgagtcactccctgatttcttgcagactctcaaagaaggaaactaaagacccgtcagtgcctttctttctgccctgctggtgtgccaatcagggatcctaacatcagggagaggacttcctgttgcagcgaagacctctgcaatgcagcagttcccactgcag

SEQ ID NO: 179 - DNA, 802 bp, synthetic construct
  misc_feature 1..802: note = Mus musculus Cox7b 3'-UTR NM_025379
  source 1..802: mol_type = other DNA
tcgtgccagctggtacaataatcaaggaattgtttaaaaccaacttataagtgaatgccaagtcaaagaatcatgtactcattatactatggcagattgaagaacaaataaagaaataaagtaccttaaccttcattctaggctttgtttttttcctttgtaaatgaagcccaagcatggtgacttctcatttatttaagctgtattgtctcttaaaatggctttttaccctatgaggtggtatgagggaaatctatgatcaggagggcacctttatagtaagctgaaattacagagaatgaagaaataagcacagagctgttttaggagcccactgggtcattggccatataggttatgcttactgccctctacctcgtggttatatttggaattgccattagctcccttctgcttagagactggactgtcaccaaacccaaggggatagtgatcctgtaatgatcctgtgtgaactaggtttgctaaagactaccacctccttacactgtatggcatattcatctgaaataggtgctaatttttcagcataatccttaatctttaggatctgtcatacttcctagtaatttaactgttgctgaagaaataaaggctatctgttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 180 - DNA, 231 bp, synthetic construct
  misc_feature 1..231: note = Mus musculus Ppib 3'-UTR NM_011149
  source 1..231: mol_type = other DNA
agagcctgggggacctcatccctctaagcagctgtctgtgtgggtcctgtcaatccccacacagacgaaggtagccagtcacaaggttctgtgccaccctggccctagtgcttccatctgatggggtgaccacacccctcacattccacaggcctgatttttataaaaaactaccaatgctgatcaataaagtgggttttttttatagcttgaaaaaaaaaaaaaaaaaaa

SEQ ID NO: 181 - DNA, 205 bp, synthetic construct
  misc_feature 1..205: note = Mus musculus Bag1 3'-UTR NM_009736
  source 1..205: mol_type = other DNA
agtgcagtggagagtggctgtactggcctgaagagcagctttacagccctgccctctctggaacagaagtcgcctgtttctccatggctgccaggggcaactagccaaatgtcaatttccctgctcctccgtcggttctcaatgaaaaagtcctgtctttgcaacctgaattagacttgtgttttctcaaaaaaaaaaaaaaaaa

SEQ ID NO: 182 - DNA, 140 bp, synthetic construct
  misc_feature 1..140: note = Mus musculus S100a4 3'-UTR S100a4-201 ENSMUST00000001046
  source 1..140: mol_type = other DNA
agactcctcagatgaagtgttggggtgtagtttgccagtgggggatcttccctgttggctgtgagcatagtgccttactctggcttcttcgcacatgtgcacagtgctgagcaaattcaataaaaggttttgaaactatt

SEQ ID NO: 183 - DNA, 374 bp, synthetic construct
  misc_feature 1..374: note = Mus musculus Bcap31 3'-UTR NM_012060
  source 1..374: mol_type = other DNA
aggcttggtgtttccctgcctgccgctggcttctacctgacccatgcttactgcttccttggagcccagactatccctctggtacttgggtttattccctacttccccaattttcttccatggcttatagatcattattttggcaccattacacatactgctcttataccaaaagggacctgattgttgtttattcagagtacttttgccactgttctgcctggctagggcactttccactcctggaagtgtagaaaagcactggtgacctggcctgcagtttgaacccctttttattttgcaatgtaccctaaaggaggctgctgtgaagcaggtcaactgttttatcctgaggggaataaatgttgttatgt

SEQ ID NO: 184 - DNA, 126 bp, synthetic construct
  misc_feature 1..126: note = Mus musculus Tecr 3'-UTR NM_134118
  source 1..126: mol_type = other DNA
gcagctcctcacggctctgcccagtaatactctccacccctcactgcccctgtcctgatgtgtggctggccatggctctccagcagcaacaataaaacctgcttacccaaaaaaaaaaaaaaaaaa

SEQ ID NO: 185 - DNA, 171 bp, synthetic construct
  misc_feature 1..171: note = Mus musculus Rabac1 3'-UTR NM_010261
  source 1..171: mol_type = other DNA
agtgtcctccaggacctgccggcctctcctgccggccggctgtcccatctctgtctgttctcgtcctacctggccttgctgctcagctccgagccttccacctgaggcctcaaacccagggaggggcttttgtctttggaaataaagctgttacaattgctatttggccaa

SEQ ID NO: 186 - DNA, 144 bp, synthetic construct
  misc_feature 1..144: note = Mus musculus Robld3 3'-UTR NM_031248 (Lamtor2)
  source 1..144: mol_type = other DNA
cagcgtgatggaggctggagtagaaaagggatgatgatctggagggaggggcggggccctagaaacgccatatcgggcgaggtacaggaagggggggttgcttttttctgaataaattttcaactcttaaaaaaaaaaaaaaaa

SEQ ID NO: 187 - DNA, 80 bp, synthetic construct
  misc_feature 1..80: note = Mus musculus Sod1 3'-UTR NM_011434
  source 1..80: mol_type = other DNA
acattccctgtgtggtctgagtctcagactcatctgctaccctcaaaccattaaactgtaatctgaaaaaaaaaaaaaaa

SEQ ID NO: 188 - DNA, 262 bp, synthetic construct
  misc_feature 1..262: note = Mus musculus Nedd8 3'-UTR NM_008683
  source 1..262: mol_type = other DNA
agaaacttggttccgtttacctccttgccctgccaatcataatgtggcatcacatatcctctcactctctgggacaccagagccactgccccctctcttggatgcccaatcttgtgtgtctactggtgggagaatgtgaggaccccagggtgcagtgttcctggcccagatggcccctgctggctattgggttttagtttgcagtcatgtgtgcttccctgtcttatggctgtatccttggttatcaataaaatatttcctg

SEQ ID NO: 189 - DNA, 257 bp, synthetic construct
  misc_feature 1..257: note = Mus musculus Higd2a 3'-UTR NM_025933
  source 1..257: mol_type = other DNA
gtatagccgggtcttaaagcgccatggaaaccattacaaaacccaggaacaacagacatccctgtcagacttgctccctccgtttcagaccggaccttattgtcatttgggtgaggaagtggccgattttgtaactgatttgcgcttccaccgctgccccctcccgctcccaaaatcccaggttcatttcagttgggttgcatgcttctatttgtgatgcgtccccttaattacttaataaaagcttattacacttg

SEQ ID NO: 190 - DNA, 268 bp, synthetic construct
  misc_feature 1..268: note = Mus musculus Trappc6a 3'-UTR Trappc6a-001 ENSMUST00000002112
  source 1..268: mol_type = other DNA
ggaccccagaccccaggcttgcccttccctaagcttagcctcggaatgtggcacctgaccctgcctcactgctcacctttgcaggtcgccttgaagctggagctcacaggctctggggaggtcacatgtgcttcagacaagggaatgaaagggccgggagggtcccgggaggtgggaccatcccctgagttccaagtcagcatggagggacattagggcatcacccagatgacagatgttcagtaaaggttctttatgtgcaaacaga

SEQ ID NO: 191 - DNA, 188 bp, synthetic construct
  misc_feature 1..188: note = Mus musculus Ldhb 3'-UTR Ldhb-001 ENSMUST00000032373
  source 1..188: mol_type = other DNA
ctgccagtctctaggctgtagaacacaaacctccaatgtgaccatgaacctttagtcttcagccatgtatgtaggtcacagtttgcttcttccctgacatgtgatatgagctcacagatcaaagcccaggcttgtttgatgtttgcactaggagctcctgatcaaataaagttagcaattgcagcata

SEQ ID NO: 192 - DNA, 112 bp, synthetic construct
  misc_feature 1..112: note = Mus musculus Nme2 3'-UTR Nme2-001 ENSMUST00000021217
  source 1..112: mol_type = other DNA
acatgaagaaaccagaatccttttcagcactactgatgggtttctggacagagctcttcatcccactgacaggatggatcatcttttctaaaacaataaagactttggaact

SEQ ID NO: 193 - DNA, 119 bp, synthetic construct
  misc_feature 1..119: note = Mus musculus Snrpg 3'-UTR NM_026506
  source 1..119: mol_type = other DNA
cctgtgctcagcaagcagtgtccacatccctccccaaaggcctgtttgattgtgatgtagaattaggtcatgtacattttcatatggaactttttactaaataaacttttgtgatactc

SEQ ID NO: 194 - DNA, 235 bp, synthetic construct
  misc_feature 1..235: note = Mus musculus Ndufa2 3'-UTR NM_010885
  source 1..235: mol_type = other DNA
aggtctccactgaggactgtgagcgagagcagctgaacctgctggactgaagacagtgtggggaaatgtgtgctttgggtccttataaagcttacgctgtacagtgtcccttcagaatgtcctcttcattaccttctccctcttactgcgcaacactgaggcaaagtagttttatataaaaatactcctttatttctcctcaaaaaaaaaaaaaaaaaaaacccaccaggtgcca

SEQ ID NO: 195 - DNA, 196 bp, synthetic construct
  misc_feature 1..196: note = Mus musculus Serf1 3'-UTR Serf1-003 ENSMUST00000142155
  source 1..196: mol_type = other DNA
tgactggctttttggaaaacctgggtgctattgccagtgggtgcatcatacgctctaagattaaaatttcacagtgactaatcattatatgtgttataacttgtccttataaaactattttaaactttactcttcagcctatcttaatgtgatgttttaagaccatcaaaaaataaagtactgaccttgcatgtaa

SEQ ID NO: 196 - DNA, 286 bp, synthetic construct
  misc_feature 1..286: note = Mus musculus Oaz1 3'-UTR Oaz1-001 ENSMUST00000180036
  source 1..286: mol_type = other DNA
gtgccagccctgcccagtgtccctgtgccctctcctgggttagtccacatgtcgtgattgtgcagaataaacgctcactccattagcggggtgcttcttcgagctgaatgctgtgtttgtcacactcaagtgttggctttaattctaaataaaggtttctattttacttttttattgctgtttaagatggtcaggtgacctatgctatagcagtctcctttgaagtctggaaaaatagtgtcacctcccctggctcaaatccaataaagtgatctcgttcattggc

SEQ ID NO: 197 - DNA, 418 bp, synthetic construct; identical to SEQ ID NO: 15
  misc_feature 1..418: note = Mus musculus Ybx1 3'-UTR Ybx1-001 ENSMUST00000079644
  source 1..418: mol_type = other DNA

SEQ ID NO: 198 - DNA, 421 bp, synthetic construct
  misc_feature 1..421: note = Mus musculus Ybx1(v2) 3'-UTR with mutation T128bpG and deletion del236-237bp
  source 1..421: mol_type = other DNA
tttttatgccggcttaccatctctaccatcatccggtttggtcatccaacaagaagaaatgaatatgaaattccagcaataagaaatgaacaaagattggagctgaagaccttaagtgcttgctttttgcccgctgaccagataacattagaactatctgcattatctatgcagcatggggtttttattatttttacctaaagatgtctctttttggtaatgacaaacgtgttttttaagaaaaaaaaaaaaggcctggtttttctcaatacacctttaacggtttttaaattgtttcatatctggtcaagttgagatttttaagaacttcatttttaatttgtaataaagtttacaacttgattttttcaaaaaagtcaacaaactgcaagcacctgttaataaaggtcttaaataataa

SEQ ID NO: 199 - DNA, 798 bp, synthetic construct
  misc_feature 1..798: note = Mus musculus Sepp1 3'-UTR NM_009155
  source 1..798: mol_type = other DNA
attatttaaaacaaggcatacctctccccaactcagtctaaagacacaatttcattttgagaatgtttacagcccatttaattaatcagtgaactaaaagtcatagaaattggatttgtgcaaatgtagagaaatctaccatattggcttccaaaatttaaaaattttatgccacagaacatttcatccaaatcagatttgtacaatagggcacctgaaaagtgactgcagcctttggttaatatgtctttctttttcctttttccagtgttctagttacattaatgagaacagaaacataaactatgacctaggggtttctgttggatagcttgtaattaagaacggagaaagaacaacaaagacatattttccagttttttttttctttacttaaactctgaaaacaacagaaactttgtcttcctactcttacattctaaaccgatgaaatctttaacagattacactttaaatatctactcatcattttctctctcagagtcctagcttgagttgcactgcatgtatctgtgcatcttgttctcttcatttaatgctgtactgttctgctgagctctgagggactatcttgagagatgtaatggaaggaaagcgtggtgttaatctgcgtactgcttaagacagtatttccataatcaatgatggtttcatagagaaactaagtcctatgaacctgacctcttttatggctaatacgactaagcaagaatggagtacagaattaagtggctacagtacacacttatcaaaataaatgcaattttaaaacctttc

SEQ ID NO: 200 - DNA, 390 bp, synthetic construct
  misc_feature 1..390: note = Mus musculus Gaa 3'-UTR Gaa-001 ENSMUST00000106259
  source 1..390: mol_type = other DNA
gagagtccgtcgtttacagaggcctccagggaggcagagggagcttgagctggctctggctggtggctcctgtaaggacctgcgtcctgctctcctgacacatctttgagcttttcccaccgtgttactgcatgcgcccctgaagctctgtgttcttaggagagtgaggctcgcctcacctgccccaccccagctgtctgtccctcacctggcactagagaatgtggagctcggcgtggggacatcgtgtctgcaccaacatcaggctgtgcagccactgcagccgcaaccctgcagagacagagctggtgccttcaccaggttcccaagactcgagaaacttactgtgaagtgtacttacttttaataaaaaggatattgtttggaagc

SEQ ID NO: 201 - DNA, 481 bp, synthetic construct
  misc_feature 1..481: note = Homo sapiens ACTR10 3'-UTR ACTR10-002 ENST00000254286
  source 1..481: mol_type = other DNA
aagtttgattaaaaatcaaccttgcttcatatcaaatatttaaccaattataagcaaattgtacaaagtatgtaggatgttttgttatagaggactatagtggaagtgaaagcattctgtgtttactctttgcattaatatataattcttttgactttgtttctcttgtgtagtggtaaaatggtagctggtgcttattgagatttgctgtatttatatcaataaagtatagtaaagcagtttgattttggaagtttgttatgtggctttttttttttttttttttttgagacggagtctcgctctgtcacttaggctggagtgcagtggcacaatctctactcattgcaagctccgcctcccgggtttacgccattctgtctcagcctcctgagtagctgggactataggcatacgccaccccgcccggctaattttttgtatatttagtagagacggggtttcaccatgttagccagga

SEQ ID NO: 202 - DNA, 347 bp, synthetic construct
  misc_feature 1..347: note = Homo sapiens PIGF 3'-UTR NM_173074
  source 1..347: mol_type = other DNA
gtaacttaatcctgacaaccgtagtgcaaggtatggcccatctcctgtacgcttggagcgacctttggctacgtggctggccttgttatttcaccactctggatatactggaatagaaagcaacttacatacaagaacaattaactggagcaaagggagatatttctttgtgcagattctgtaagggctgggcagaaatgtgtatggtcaaagccaagcagttccatttacagctctgttttttacgtagttacaacatgatgtgattgtagctttttaaactatgaaacccctgagagattgtaccttctagttgaaataaagtatttataatagattgtggcttc

SEQ ID NO: 203 - DNA, 233 bp, synthetic construct
  misc_feature 1..233: note = Homo sapiens PIGF 3'-UTR NM_002643.3
  source 1..233: mol_type = other DNA
ctggagcaaagggagatatttctttgtgcagattctgtaagggctgggcagaaatgtgtatggtcaaagccaagcagttccatttacagctctgttttttacgtagttacaacatgatgtgattgtagctttttaaactatgaaacccctgagagattgtaccttctagttgaaataaagtatttataatagattgtggcttcaaaaaaaaaaaaaaaaaaaaaaaaaaaaaa

SEQ ID NO: 204 - DNA, 116 bp, synthetic construct
  misc_feature 1..116: note = Homo sapiens MGST3 3'-UTR MGST3-001 ENST00000367889
  source 1..116: mol_type = other DNA
agaattataggggtttaaaaactctcattcattttaaatgacttacctttatttccagttacattttttttctaaatataataaaaacttacctggcatcagcctcatacctaaaa

SEQ ID NO: 205 - DNA, 913 bp, synthetic construct
  misc_feature 1..913: note = Homo sapiens SCP2 3'-UTR NM_001193599
  source 1..913: mol_type = other DNA
agaactccctttggctacttttgaaaatcaagatgagatatatagatatatatccatacattttattgtcagaatttagactgaaactacacattggcaaatagcgtgggatagatttgtttcttaatgggtgtgaccaatcctgtttttcctatgctctgggtgaatagagcctgatggtatactactgctttgcggaattgcatacaactgtgcattacaaagttaatatggtaattatggtctggggtaaaattgagtttcagaataaaattaggaacagtaaaatccaaagaactatgtaaacaaaaaagcttttgttttgcttacaaagtatatttaaggattattctgctgaagattcagtttaagagttttccttgggagaactaagtaagaaacacaatgccaacagctggccagtaattagtgttgtgcacttcatgtcattaatcaatttctcaatagttcttaaaattagtgagattaaaaatctaaaaattttgcatttcatgctatcagaaacagtattttcttcccaaatcaaaataaaagaaatatgatcagagcttgaacacaggcttatttttaaaataaaaatatttttaacatgggtttccttattgaaaaatcagtgtattagtcataaaacaccatcattaagaataattgaacaataaagtttgctttcagatgcagttttcaaattataatctcatttcaatttataacgttctcagtcctttgttataattttcctttttcatgtaagtttaattatctgcatttatcttttttcctagtttttctaatactaatgttatttcttaaaattcagtgagatataggataaaataatgctttgagaagaatgtttaatagaaaattaaaataactttttctggcctctctt

SEQ ID NO: 206 - DNA, 409 bp, synthetic construct
  misc_feature 1..409: note = Homo sapiens SCP2 3'-UTR SCP2-015 ENST00000435345
  source 1..409: mol_type = other DNA
agaactccctttggctacttttgaaaatcaagatgagatatatagatatatatccatacattttattgtcagaatttagactgaaactacacattggcaaatagcgtgggatagatttgtttcttaatgggtgtgaccaatcctgtttttcctatgctctgggtgaatagagcctgatggtatactactgctttgcggaattgcatacaactgtgcattacaaagttaatatggtaattatggtctggggtaaaattgagtttcagaataaaattaggaacagtaaaatccaaagaactatgtaaacaaaaaagcttttgttttgcttacaaagtatatttaaggattattctgctgaagattcagtttaagagttttccttgggagaactaagtaagaaacacaatgc

SEQ ID NO: 207 - DNA, 591 bp, synthetic construct
  misc_feature 1..591: note = Homo sapiens HPRT1 3'-UTR HPRT1-001 ENST00000298556
  source 1..591: mol_type = other DNA
gatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaa

SEQ ID NO: 208 - DNA, 283 bp, synthetic construct
  misc_feature 1..283: note = ACSF2 Homo sapiens
  source 1..283: mol_type = other DNA
ataaagcagcaggcctgtcctggccggttggcttgactctctcctgtcagaatgcaacctggctttatgcacctagatgtccccagcacccagttctgagccaggcacatcaaatgtcaaggaattgactgaacgaactaagagctcctggatgggtccgggaactcgcctgggcacaaggtgccaaaaggcaggcagcctgcccaggccctccctcctgtccatcccccacattcccctgtctgtccttgtgatttggcataaagagcttctgttttctttg

SEQ ID NO: 209 - DNA, 555 bp, synthetic construct
  misc_feature 1..555: note = Homo sapiens VPS13A 3'-UTR NM_033305
  source 1..555: mol_type = other DNA
aattcatatgttctttattttacttggaatgtttcattaacatgttttgtatgacttataccataatgcccatatgtccatttatagggaggtaaaacacattttcttttaaaatgttttcctacacattttcataaagcaaaataattgtattatttaagcacagaaaaaaatgtatcttacatccaaagtagggagggcatccaacatattatagatttgcttttatatattttatagctttgtattgcatagtttgtctttaagagttcaagttagacttaaatataattttgatgttcactggttttattttaaattgccttcttatttgttagcaaaatgcctttttttaatggtctctgtaaattttctgggctttaatgtaatgccactgtgtaaaaaaaaaggaagaaaatagtaatagccatttaatgttttatatttatcattttaaagatatttttgtcaaatttcttttaataataataaacatatgtaatctaaaaaaaaaaaaaaaaaaaaaaaaaaaaaaaaaaaaaaaaaaaaaaaaaa

SEQ ID NO: 210 - DNA, 724 bp, synthetic construct
  misc_feature 1..724: note = Homo sapiens CTH 3'-UTR NM_001190463.1
  source 1..724: mol_type = other DNA
tattccagagctgctattagaagctgcttcctgtgaagatcaaatcttcctgagtaattaaatggaccaacaatgagcctttgcaaaattttcaagcggaaattttaaggcacctcattatctttcataactgtaattttcttagggatcatctctgttaaaaagttttctgtatgtcatgttataattacaggtcaattctgttaatatctttttgttaattttgctctatgtttgcctctgaaggaggtgagatttgtgctactttgggagattatgttcttttttcatgtctaagatttattttgatcatgtttataatataatggtaattcatttttgatgttttgtgaagaatttaaatttaaacgaatgttcttaaatcaagtgtgatttttttgcatatcattgaaaagaacattaaaagcaatggtttacacttagttaccataagccgaaaatcaaatacttgaaaagtttactgtgaaattctactgatttaagactatacttaatatttttaaaaaaataaatcagctgggcgcggtggctcacgcatgtaatgccagcacttttggaggataaggcgggcggatcacgaggtcaggagattgagaccatcctggctagcgcagtgaaacccccatctctactaaaaatgcaaaaaaaattagacggacgtggtggcgggtgcctgtagtcccagctacttgggaggctgagg

SEQ ID NO: 211 - DNA, 443 bp, synthetic construct
  misc_feature 1..443: note = Homo sapiens CTH 3'-UTR CTH-001 ENST00000370938
  source 1..443: mol_type = other DNA
tattccagagctgctattagaagctgcttcctgtgaagatcaaatcttcctgagtaattaaatggaccaacaatgagcctttgcaaaattttcaagcggaaattttaaggcacctcattatctttcataactgtaattttcttagggatcatctctgttaaaaagttttctgtatgtcatgttataattacaggtcaattctgttaatatctttttgttaattttgctctatgtttgcctctgaaggaggtgagatttgtgctactttgggagattatgttcttttttcatgtctaagatttattttgatcatgtttataatataatggtaattcatttttgatgttttgtgaagaatttaaatttaaacgaatgttcttaaatcaagtgtgatttttttgcatatcattgaaaagaacattaaaagcaatggtttacactta

SEQ ID NO: 212 - DNA, 77 bp, synthetic construct
  misc_feature 1..77: note = Homo sapiens CTH 3'-UTR CTH-002 ENST00000346806
  source 1..77: mol_type = other DNA
tattccagagctgctattagaagctgcttcctgtgaagatcaaatcttcctgagtaattaaatggaccaacaatgag

SEQ ID NO: 213 - DNA, 286 bp, synthetic construct
  misc_feature 1..286: note = Homo sapiens NXT2 3'-UTR NXT2-004 ENST00000372107
  source 1..286: mol_type = other DNA
aggggcaaaagtccattctcatttggtccattagttccagcaattgaaatttatgtgaattattttgattgtagaagcactataatatgtgctgaaactaaatttctttaatattttctattcctgtcagcaccttttctagcagctgccagtttggagcattgccctctaagagctttaaaactatttttttacatgccttatatacattccactaatgacattcttataataatattaaacacatgatcttggtactaacatactcactgtgaacccagcctat

SEQ ID NO: 214 - DNA, 121 bp, synthetic construct
  misc_feature 1..121: note = Homo sapiens MGST2 3'-UTR NM_002413
  source 1..121: mol_type = other DNA
ctttttctcttccctttaatgcttgcagaagctgttcccaccatgaaggtaatatggtatcatttgttaaataaaaataaagtctttattctgtttttcttgaaaaaaaaaaaaaaaaaaa

SEQ ID NO: 215 - DNA, 582 bp, synthetic construct
  misc_feature 1..582: note = Homo sapiens MGST2 3'-UTR NM_001204366.1
  source 1..582: mol_type = other DNA
ctttttctcttccctttaatgcttgcagaagctgttcccaccatgaaggcttgaagccacagtgcatggccagaaccagccagacctttggagttcaagaactcgagaggtgggtgaaaactgccattgcctccacagactgtcttctccgtggaaagaagacctgagtcaccagggctgggaaacctgcaccactgagacgagcacagcctctgccggcatgcaagtggccgctgtcaggacacatggactgaaagtggtttgtcagctgctccattaggttttttttacccatatgtttgctacctttctttccttgatttaaaaatagggagggggagcagtctcagctgtcttcagctgctagggagatttttttccccctcctgagctactgtttcccccaacccgagcctttctctcttattgtacccaccctttctgatgaagtcatcaaagcaaagattgcataactgatgcataggcctatcttgtgttatactgggagacaggccaatgtttccattaatagacaagagcaccaccacgctgccaaatggagctctctgctgcaaccactac

SEQ ID NO: 216 - DNA, 37 bp, synthetic construct
  misc_feature 1..37: note = Homo sapiens C11orf67 3'-UTR AAMDC-005 ENST00000526415
  source 1..37: mol_type = other DNA
tggagccttaagaggagaataaatcactaagtgccta

SEQ ID NO: 217 - DNA, 266 bp, synthetic construct
  misc_feature 1..266: note = Homo sapiens PCCA 3'-UTR NM_000282
  source 1..266: mol_type = other DNA
aggatttataacctttcagtcatcacccaatttaattagccatttgcatgatgctttcacacacaattgattcaagcattatacaggaacacccctgtgcagctacgtttacgtcgtcatttattccacagagtcaagaccaatattctgccaaaaaatcaccaatggaaattttcattgatataaatacttgtacatatgatttgtacttctgctgtgagattccctagtgtcaaaattaaatcaataaaactgagcatttgtct

SEQ ID NO: 218 - DNA, 142 bp, synthetic construct
  misc_feature 1..142: note = Homo sapiens GLMN 3'-UTR NM_053274
  source 1..142: mol_type = other DNA
aagttccatttcctaaataaaaactaataaaatatagtactttccattatgattcatttaatacctttataaaaaatttttctgtaaaaatttactgcttgaaaaataaatgtagcttttctcatttatcaaaaaaaaaaaa

SEQ ID NO: 219 - DNA, 276 bp, synthetic construct
  misc_feature 1..276: note = Homo sapiens DHRS1 3'-UTR NM_001136050
  source 1..276: mol_type = other DNA
ccctcctggtctgacactacgtctctgcttgtcttctcatttggacttggtggttcgtcctgtctcagtgaaacagcagcctttcttgtttacccatacccttgatatgaagagaagccctctgctgtgtgtccgtggtgagttctggggtgcgcctaggtcccttctttgtgccttggttttccttgtccttctttttactttttgccttagtattgaaaaatgctcttggagctaataaaagtctcatttctctttcaaaaaaaaaaaaaaaaa

SEQ ID NO: 220 - DNA, 450 bp, synthetic construct
  misc_feature 1..450: note = Homo sapiens PON2 3'-UTR PON2-001 ENST00000433091
  source 1..450: mol_type = other DNA
attgtacttttggcatgaaagtgcgataacttaacaattaattttctatgaattgctaattctgagggaatttaaccagcaacattgacccagaaatgtatggcatgtgtagttaattttattccagtaaggaacggcccttttagttcttagagcacttttaacaaaaaaggaaaatgaacaggttctttaaaatgccaagcaagggacagaaaagaaagctgctttcgaataaagtgaatacattttgcacaaagtaagcctcacctttgccttccaactgccagaacatggattccactgaaatagagtgaattatatttccttaaaatgtgagtgacctcacttctggcactgtgactactatggctgtttagaactactgataacgtattttgatgttttgtacttacatctttgtttaccattaaaaagttggagttatattaa

SEQ ID NO: 221 - DNA, 277 bp, synthetic construct
  misc_feature 1..277: note = Homo sapiens NME7 3'-UTR NM_013330
  source 1..277: mol_type = other DNA
tggtgtggaaagtaaagaagtcacaggttgggacatttagacaagagtgaatcacacacgaggaatgtgttcattcttttattgtccgttgttttaacctgactgaatacaagatcaacaagagcactgtactcctggcaattattacatatgttagaacatggattttgcactgtagacaacatttaacaccagtctatggggtactgcattgctttttataaagttcaaaataaagatttattttcaaacaaaaaaaaaaaaaaaaaaaaaaaaa

SEQ ID NO: 222 - DNA, 163 bp, synthetic construct
  misc_feature 1..163: note = Homo sapiens ETFDH 3'-UTR NM_004453
  source 1..163: mol_type = other DNA
actgcagctagccagtttctttcaagtatggcaagctaacgttaaaatgtttagagattaacagatttcagaatgtctttctgcatattactgaacagaatagtcacaaaatgattatcaaataaaaattttatactatatgtaagattgtcccataaagaaa

SEQ ID NO: 223 - DNA, 275 bp, synthetic construct
  misc_feature 1..275: note = Homo sapiens ALG13 3'-UTR BC117377
  source 1..275: mol_type = other DNA
gatccagcagtatgaagtattcttgcactgccattttcttgctgtttttgtttttaaaaagtattttatgttagtggttaaatgatttaggtgattagtgtttactattgtatttgtctttaaaattattttatcttttgatttaaaatagtactttaaaattaaggggtattattttgggctgtgactaaggaaattgagatggatgtacaactagccccatattgagcatacttcattgtattcagctgttttcctgtcagccatttgtcagc

SEQ ID NO: 224 - DNA, 664 bp, synthetic construct
  misc_feature 1..664: note = Homo sapiens ALG13 3'-UTR NM_001099922.2
  source 1..664: mol_type = other DNA
gatccagcagtatgaagtattcttgcactgccattttcttgctgtttttgtttttaaaaagtattttatgttagtggttaaatgatttaggtgattagtgtttactattgtatttgtctttaaaattattttatcttttgatttaaaatagtactttaaaattaaggggtattattttgggctgtgactaaggaaattgagatggatgtacaactagccccatattgagcatacttcattgtattcagctgttttcctgtcagccatttgtcagctttatattagctgatggtaccaattgataaaatgaatataaagtatttcattggttcaaaaatcacacatcatattaaaccatgcagaattggagtaacttccacttttttctagaaagtaaaaccaagagcctttgcttctggataactcacttaatattaaattaaagagctcttcacgtttcttgagaattatctgaagccagttgcattctgtgatatcagttttgaaggcacatggttctctgctttagatttatcccatatgctattgtttaatactggatgtatgtaagtgttttactgcactgtattgaattggtgtcttttgcacagttagcagtaaataaaaattagcatttaaaattgccaaaaaaaaaaaaaaaaaa

SEQ ID NO: 225 - DNA, 640 bp, synthetic construct
  misc_feature 1..640: note = Homo sapiens DDX60 3'-UTR DDX60-001 ENST00000393743
  source 1..640: mol_type = other DNA
aaacaaagtctatgcaaaccacttaaaaataattccatagtagtttttcaggtcacgtttttgattcttatgcttcttgccagaaatacattatgataaagtggaaatacattacgatgaagtggaaagagcaaacactttggaatcaaacagagttgcaatcaaacctgccatgttctgtcatgaatactcacaaattatttagtatacctgaatcttggtttctttttataactgagtaataatggttacatctcaggtagtttgaggattgactaaaaaaatgcgagaatgttgtatgtgactgaataacaatttttactctgcgaagccaaagtaaatataatattatcagtaactttatccccagtgtcagtatttataaaatgtttattaaggctagaaaaaatgaatacaatatcctgaaggtgaaatatattctcttcaattagcataaatatgatttacataagttagctatacagctattgagatagtactttctagtaaacttaaactactttttaaacatacattttgtgatgatttaacaaaaatatagagaatgatttgctttattgtaattgtatataagtgactggaaaagcacaaagaaataaagtgggttcgatctgtttac

SEQ ID NO: 226 - DNA, 451 bp, synthetic construct
  misc_feature 1..451: note = Homo sapiens DYNC2LI1 3'-UTR NM_015522.3
  source 1..451: mol_type = other DNA
aattcatttgatgtagatgaacctgttcactggaaaattacagcaatttattaaaacctcagtaagagcaaaacaaggaagaagattccttatatcttcttgttagacatcttctgtgattgttatggcatattacaccaatcagagaaatagagttttaaagtagtggtttgatattgattttataatctctgtaaaaatgaagataaaaagccagattgtacaaaagtcacctgacaaagactagatgaagctacaactttaagcaaggggtagagttgtaatagccttcaccatcactctgtattttacattcatttcgtttctgtcacttattcagtatctttttatcatctgacagctaattaaattataaagttgctatgatggtaacacaagttcttcaaatacaataataaatatcatcatctggaaaaaaaaaaaaaaaaaa

SEQ ID NO: 227 - DNA, 606 bp, synthetic construct
  misc_feature 1..606: note = Homo sapiens VPS8 3'-UTR NM_001009921, NM_015303
  source 1..606: mol_type = other DNA
tgactccatggagcctggcccaggagaaccagagatgatcccgaggcagctggggagaggccccgcctctggtgggcttggcctccaccacctcccacgcttctgagaagaggttccaaattgggcttctgtgcccagagcgtccacagcaccattcccagtgtagactcccagtcttctccacattgctgtcatggcgtcagttcaccagactcattgattttgttttgcttgttaagcaaaggaatgtcacatacctctgtccagctttttaggaaatacatttcgcctattgcgactttttccatttaccctgaagcctagaaagtaggtggaactcacacaaatggcattccagagtctgccatactccgtctcctccagctgctggataatacagaggaacttcaacttctacagggaacagtggttggccaggctgcagtataactgaagcatgccttggagagagcagacactgtgggggccagggccatctccctttaatgtgttcatgttaaaacctatttgagtgtaagacttgccctttctaacaataaatgctccgtgtttaagttctgcaggtctcaaaaaaaaaaaaaaaaa

SEQ ID NO: 228 - DNA, 360 bp, synthetic construct
  misc_feature 1..360: note = Homo sapiens ITFG1 3'-UTR NM_030790
  source 1..360: mol_type = other DNA
cttgcctttaatattacataatggaatggctgttcacttgattagttgaaacacaaattctggcttgaaaaaataggggagattaaatattatttataaatgatgtatcccatggtaattattggaaagtattcaaataaatatggtttgaatatgtcacaaggtctttttttttaaagcactttgtatataaaaatttgggttctctattctgtagtgctgtacatttttgttcctttgtggaatgtgttgcatgtactccagtgtttgtgtatttataatcttatttgcatcatgatgatggaaaaagttgtgtaaataaaaataattaaaaaaaaaaaaaaaaaaaaaaaaaaaaaa

SEQ ID NO: 229 - DNA, 215 bp, synthetic construct
  misc_feature 1..215: note = Homo sapiens CDK5 3'-UTR NM_004935
  source 1..215: mol_type = other DNA
gccccgggacccccggcctccaggctggggcctggcctatttaagccccctcttgagaggggtgagacagtgggggtgcctggtgcgctgtgctccagcagtgctgggcccagccggggtggggtgcctgagcccgaatttctcactccctttgtggactttatttaatttcataaattggctcctttcccacagtcaaaaaaaaaaaaaaaaaa

SEQ ID NO: 230 - DNA, 146 bp, synthetic construct
  misc_feature 1..146: note = Homo sapiens C1orf112 3'-UTR BC091516
  source 1..146: mol_type = other DNA
aacttatcactaggcagaactgggtttgatgctttgtcaactgaaaatacttatgtctgtacattttctaacagatataaaacaaattttgtaaagttgaaaaaaaaaaaaaaaaaaaaaaaaaaaaaaaaaaaaaaaaaaaaaaa

SEQ ID NO: 231 - DNA, 239 bp, synthetic construct
  misc_feature 1..239: note = Homo sapiens IFT52 3'-UTR NM_016004
  source 1..239: mol_type = other DNA
agaccatgcctcttgaagctttttctgcctcctgattctctctttgtaaactattttcaaattgtttttcaactccttatcaaaattgtttatacactctttcctccatgagctctggaaggtatatgcatcttctgtaatactcagataggtataagatttttcacaaaatccttatgtaagatacattccatttttaaaaattaaatgtatggttgcatctgtctttttatacccta

SEQ ID NO: 232 - DNA, 146 bp, synthetic construct
  misc_feature 1..146: note = Homo sapiens CLYBL 3'-UTR CLYBL-003 ENST00000339105
  source 1..146: mol_type = other DNA
tctgttaaatgaagctgtcatcaggctaaagggtattgaagctgcagagggatcaacttgtgcttgccagaggacgccaatgaagtttgaaacaccaacaatcagagattttgtttctgttcctcattaaatcatgagcttttgtg

SEQ ID NO: 233 - DNA, 477 bp, synthetic construct
  misc_feature 1..477: note = Homo sapiens FAM114A2 3'-UTR FAM114A2-006 ENST00000520667
  source 1..477: mol_type = other DNA
agaatggagacgttttgacctgggacttgtgacggccaaggaatgccaccttattctggctactcctgcagaaatgaaggagtggggttattttagtatataaaaattcaggcaggagagatggtttaaagaggaagattgttgccttcagtgtttgattgaagtattcaggttctcacagtattctttccagttgttgtaattcataaattatttgaaaagaaacttttgtagaaagtccaagaataataactctagataaagattagtgggacactcaggcaaaaatgttggtctttctttgacatgttgcaaaatgttatcaattttgtcatggatataatttgcagcccatggatataactggttgataagccagagaaaaataatttagtgttctaaaattcatggcatgtgtggtttattaatgccatgtactttctcctttctggaataaaatctatggctttaagaaaa

SEQ ID NO: 234 - DNA, 310 bp, synthetic construct
  misc_feature 1..310: note = Homo sapiens NUDT7 3'-UTR NM_001243661
  source 1..310: mol_type = other DNA
tttactagagcaagagacaaagaactattcacgaggattctgtgtgtgcttattcgtagaacaacaacaatgccagctgttggaatttgacaggtgtgaatattttttctgcagtatgtagttagaatccttgcctcttttccagttgccttctattgtctgaaaaagtaaaagccattcaaaaatgaaaactatgttcatagtgttgcatattttcacccacaatatgttaataatatttttcttacacatataataaagaatatctggcacatactaggcccttaataaagattttttgaatatataa

SEQ ID NO: 235 - DNA, 513 bp, synthetic construct
  misc_feature 1..513: note = Homo sapiens AKD1 3'-UTR NM_001145128
  source 1..513: mol_type = other DNA
tttacttaggtgatagcagcctgaatctcaagagttatctgaaagtgatagagggaaactgagagaagtagattgaaaatctgggcctcttggaagtacttttgcctcctgagcaaggtaccatggctgccagacttcaggtgaactcaaaggtctgccagccaggaaggagcactcttatggaaacaagttttaatacaattttaaaatgtattgctctttgcctgaactttgatgctttaacaaaataaacattctatttataattccatatagaaaagttaagtgacttatttaataaatgtattattttcctttttaacattttcagtagaaaagtcagtctctgttaaaattactcattaaatgttagaaagctttaagacatttaacattgttataaatgaaaccaaaatatgggttatacattttacatacaaaactgtttgtgaactttgtgaacataagatactatcattttcccaataaaataaatggattttgcaacaactt

SEQ ID NO: 236 - DNA, 160 bp, synthetic construct
  misc_feature 1..160: note = Homo sapiens MAGED2 3'-UTR NM_014599
  source 1..160: mol_type = other DNA
gattttagatattgttaatcctgccagtctttctcttcaagccagggtgcatcctcagaaacctactcaacacagcactctaggcagccactatcaatcaattgaagttgacactctgcattaaatctatttgccatttcaaaaaaaaaaaaaaaaaaaa

SEQ ID NO: 237 - DNA, 498 bp, synthetic construct
  misc_feature 1..498: note = Homo sapiens HRSP12 3'-UTR HRSP12-001 ENST00000254878
  source 1..498: mol_type = other DNA
gtgggcccagtgctgtgtagtctggaattgttaacattttaatttttacaattgatgtaacatcttaattaaccttttaattttcacaattgatgacagtgtgagtttgatgaaaatatctgaagctattatggaaataccatgtaatagggagagttgaacatgaatattagagaaggaatccagttacttttttaaattacacctgtgtgcacctgtattactgaatataggaaagagatacccattacatagttactcagtaaacaaaagagaaataccaggtaggaaagaagagttactattcctgagaaataatcaagaacatatttaatttaaactaatgatgtgaactatttagttttgatgtccgttatgtgattctgcttttacttgagtaaaattaaagtgtttaaatttgagatcaaggagaagatagtggaacaaaatgttatatagataatatttttctaatggaaataaaataggcagatttcc

SEQ ID NO: 238 - DNA, 127 bp, synthetic construct
  misc_feature 1..127: note = Homo sapiens STX8 NM_004853 3'-UTR
  source 1..127: mol_type = other DNA
tggcagtaaagagaccaccagcagtgacacctgccaatgacagatgcaagcccaacacccttttggtacgcaaaacctgctctcaataaattcccccaaagctctgaaaaaaaaaaaaaaaaaaaaa

SEQ ID NO: 239 - DNA, 386 bp, synthetic construct
  misc_feature 1..386: note = Homo sapiens ACAT1 3'-UTR ACAT1-001 ENST00000265838
  source 1..386: mol_type = other DNA
acaacctctgctatttaaggagacaaccctatgtgaccagaaggcctgctgtaatcagtgtgactactgtgggtcagcttatattcagataagctgtttcattttttattattttctatgttaacttttaaaaatcaaaatgatgaaatcccaaaacattttgaaattaaaaataaatttcttcttctgcttttttcttggtaaccttgaaaagtttgatacatttttgcattctgagtctatacttatcgaaatatggtagaaataccaatgtgtaatattagtgacttacataagtagctagaagtttccatttgtgagaacacatttatatttttgaggattgttaaaggtcaagtgaatgctctttataggtaatttacatt

SEQ ID NO: 240 - DNA, 189 bp, synthetic construct
  misc_feature 1..189: note = Homo sapiens IFT74 3'-UTR IFT74-201 ENST00000433700
  source 1..189: mol_type = other DNA
gtttaagtccactgaaagtctctaaggaagtatcctcttgctgctaaacttggtacaagttgactaccaaaaaaaaaaaaagcttacttttggagtttacctaaaatttctgaatgttataatttttgtggcctcttttaagaatgatattttaaaatagtaaatagttcaataaatggtttgcatatt

SEQ ID NO: 241 - DNA, 361 bp, synthetic construct
  misc_feature 1..361: note = Homo sapiens KIFAP3 3'-UTR NM_014970
  source 1..361: mol_type = other DNA
taaagtatctgtttccatgtgtaatctcagcttagaagaaatctgtgtgggttgggttaattttggatctttgcctaataatgcatgttgatgttattgtgggtctgtgtttgtttttatttttatatgttgttagctgcagattaaccccagcccctctgtcttctgttaagtacagttgatactgacattgttcactcatcaaaccacatcttgatgctaagtaacatttcccatgagccacaaaactgaatgctgaaaagctactagactggaaaacaaacactgcattatgtatgttaagtgactaatttaatttcaattaaaaagcgtaaagtgaaaatgaaaaaaaaaaaaaaaa

SEQ ID NO: 242 - DNA, 783 bp, synthetic construct
  misc_feature 1..783: note = Homo sapiens CAPN1 3'-UTR NM_005186
  source 1..783: mol_type = other DNA
ggcagggactcggtcccccttgccgtgctcccctccctcctcgtctgccaagcctcgcctcctaccacaccacaccaggccaccccagctgcaagtgccttccttggagcagagaggcagcctcgtcctcctgtcccctctcctcccagccaccatcgttcatctgctccgggcagaactgtgtggcccctgcctgtgccagccatgggctcgggatggactccctgggccccacccattgccaagccaggaaggcagctttcgcttgttcctgcctcgggacagccccgggtttccccagcatcctgatgtgtcccctctccccacttcagaggccacccactcagcaccaccggcctggccttgcctgcagactataaactataaccactagctcgacacagtctgcagtccaggcgtgtggagccgcctcccggctcggggaggccccggggctgggaacgcctgtgccttcctgcgccgaagccaacgccccctctgtccttccctggccctgctgccgaccaggagctgcccagcctgtgggcggtcggccttccctccttcgctccttttttatattagtgattttaaaggggactcttcagggacttgtgtactggttatgggggtgccagaggcactaggcttggggtggggaggtcccgtgttccatatagaggaaccccaaataataaaaggccccacatctgtctgtgaaaaaaaaaaaaaaaaaaaaaaaaaaaaaaaaaaaaaaaaaaaaaaaaaaaaaaaaaaaaaaaa

SEQ ID NO: 243 - DNA, 88 bp, synthetic construct
  misc_feature 1..88: note = Homo sapiens COX11 3'-UTR NM_001162861
  source 1..88: mol_type = other DNA
agagttggcacctttgatgtggtagtgagctgatcatccactttcttctaaaataaagagaagaaaatggccagtaaaaaaaaaaaaa

SEQ ID NO: 244 - DNA, 77 bp, synthetic construct
  misc_feature 1..77: note = Homo sapiens GLT8D4 3'-UTR BC127733
  source 1..77: mol_type = other DNA
atattttgtcttgttgcaagtcaattaggtgtcttgtgaacaaggaaatactaatctctaagctgcctgggtctttt

SEQ ID NO: 245 - DNA, 215 bp, synthetic construct
  misc_feature 1..215: note = Homo sapiens GLT8D4 3'-UTR NM_001080393
  source 1..215: mol_type = other DNA
atattttgtcttgttgcaagtcaattaggtgtcttgtgaccaaggaaatactaatctctaagctgcctgggtctttttgtgtgaatatttaatggtgctccatgactgttgagttttaaaaacctcgttaaattttgccaaatcagttgcccccaaaagggaatatgcttttccttatttttttttctaaaatgctatttatctctaaggaaaaa

SEQ ID NO: 246 - DNA, 140 bp, synthetic construct
  misc_feature 1..140: note = Homo sapiens HACL1 3'-UTR NM_012260
  source 1..140: mol_type = other DNA
ataaagacgccagttggtggtcttgagttttctctttcttgcaagatgaaattttattttccacagcaaaattactctactgttaaaattgtgcaaaataaaataaacatttaaaatgacattttacagtaaaaaaaaaa

SEQ ID NO: 247 - DNA, 273 bp, synthetic construct
  misc_feature 1..273: note = Homo sapiens IFT88 3'-UTR NM_175605
  source 1..273: mol_type = other DNA
tattcactttaatatttattaaaggaaagaaattgccttatgagatcatcctcatgttaaaccttggattaaatatctaacctgtaattattttttttcactgtcaaaacttaagtaagtgtattctattctgtatgtatgcatttaagttgtttttttcttttaaggaataaaaacaggtaaaactaatactttaggccagtgacttccttagctttttgaaaacattgacacacaggaagaaataaatttcataacacaaaaaaaaaaaaa

SEQ ID NO: 248 - DNA, 187 bp, synthetic construct
  misc_feature 1..187: note = Homo sapiens IFT88 3'-UTR IFT88-001 ENST00000351808
  source 1..187: mol_type = other DNA
tattcactttaatatttattaaaggaaagaaattgccttatgagatcatcctcatgttaaaccttggattaaatatctaacctgtaattattttttttcactgtcaaaacttaagtaagtgtattctattctgtatgtatgcatttaagttgtttttttcttttaaggaataaaaacaggtaaaact

SEQ ID NO: 249 - DNA, 150 bp, synthetic construct
  misc_feature 1..150: note = Homo sapiens NDUFB3 3'-UTR NM_002491
  source 1..150: mol_type = other DNA
agataatacctggaagcatcatagtggtttcttaactctccaaaataagatttcttctctgtagcctacttgtctggtttatcccttacagaatattagtaagatttaatcaattaaaatatatatatatgccaaaaaaaaaaaaaaaaa

SEQ ID NO: 250 - DNA, 589 bp, synthetic construct
  misc_feature 1..589: note = Homo sapiens ANO10 3'-UTR NM_018075
  source 1..589: mol_type = other DNA
gtgcccagcgtgcccagctgccctgttggcagaggcctgtgtctgtgccacacctgccacggtggcagggggggtacccggggcagcatcgtggctcctgaacccagacccaatgcttagccaaacgaagtggctcccatgtggcaagcacccttctcagtttcgcagtggcttggctcgggatccttggcagttcccccagccccaccctgtctgctccttcccagttccttcccgggccccacacgctgctccagctgccaactttgctgcagagccactgccgcccttgagcctctcaccatgagtgagccaccagctctccacgttcccctcatagcagtgtcactcccaaccccaccatggcccagggacccgtggacaggttggggatggggtgtgtgcccactgtgctcatcacaggagcctcagttgagagtgagcggggtacagtaaggcagtgcttcccacactggacctctttcctggttctcttttgcaatacattaacagaccctttatcaacataaacaatagtaactgagctattaaaggcaacctctctgactccttctgcctaaaaaaaaaa

SEQ ID NO: 251 - DNA, 263 bp, synthetic construct
  misc_feature 1..263: note = Homo sapiens ANO10 3'-UTR ANO10-005 ENST00000451430
  source 1..263: mol_type = other DNA
gtgcccagcgtgcccagctgccctgttggcagaggcctgtgtctgtgccacacctgccacggtggcagggggggtacccggggcagcatcgtggctcctgaacccagacccaatgcttagccaaacgaagtggctcccatgtggcaagcacccttctcagtttcgcagtggcttggctcgggatccttggcagttcccccagccccaccctgtctgctccttcccagttccttcccgggccccacacgctgctccagctgcca

SEQ ID NO: 252 - DNA, 486 bp, synthetic construct
  misc_feature 1..486: note = Homo sapiens ARL6 3'-UTR NM_032146
  source 1..486: mol_type = other DNA
aaagataatagttggaaacctcagcaattttcaattcaaggaatctatctaagacaaatagaatacattttgtaaaagatgtttatgcatcaaaaaatataattttctgcttgcatttatggactctgacctttttaagaacataggacttcaggtatgctaatttggccattaattatttaaaaactaaatattccctcaaaagggctccctagaattatcaagttcttagtgaaggtctacatttgattgtacgtagaatgtttaaaagtcagttataagccatctcatcccatcataatttatgatatgtttaatatattttattttttaattgtctttttaaaaaatttagtttatgactttgcagtatgaattgtgcttgtgaaaaagaactttaaatatttataagggaccatgggtaattaatatatattcaatttttactatgtgtcactgtcaataaaatgtaaaatataatgtgcc

SEQ ID NO: 253 - DNA, 719 bp, synthetic construct
  misc_feature 1..719: note = Homo sapiens LPCAT3 3'-UTR NM_005768
  source 1..719: mol_type = other DNA
tccatttccctggtggcctgtgcgggactggtgcagaaactactcgtctcccttttcacagcactcctttgccccagagcagagaatggaaaagccagggaggtggaagatcgatgcttccagctgtgcctctgctgccagccaagtcttcatttggggccaaaggggaaacttttttttggagaaggcgtcttgctttgtcacccacgctggaatgcagtggcgggatctcagctcaccgcaacctccacctcctgggttcaagtgattttcctgcctcagcctcccaagtagctgggaatacaggcacgccaccatgcccagctaatttttgtattttcagtagaaacgggatttcaccacgttggccaggctggtctcgaactcctgaccgcaagtgatccacccgcctccgcctcccaaagtgctgggattacaggcgtgagccaccgtgcccggcccaaaggggaaactcttgtgggaggagcagaggggctcacatctcccctctgattcccccatgcacattgccttatctctccccatctagccaggaatctattgtgtttttcttctgccaatttactatgattgtgtatgtgccgctaccaccaccccccccatgggggggtggagaggggtgcaaggccctgcctgctccactttttctaccttggaactgtattagataaaatcacttctgtttgttcagtttttca

SEQ ID NO: 254 - DNA, 154 bp, synthetic construct
  misc_feature 1..154: note = Homo sapiens ABCD3 3'-UTR NM_001122674
  source 1..154: mol_type = other DNA
aaaccagacaaatgtattggccaggcgtggtggctcatgcctgtaatcccagcactttgggaggctgagatgggaggatcgcttgaatccaggagttcgagacaagcctggacaaaaagcgagacccgcttctttaaaaaataataataaaaca

SEQ ID NO: 255 - DNA, 448 bp, synthetic construct
  misc_feature 1..448: note = Homo sapiens COPG2 3'-UTR NM_012133
  source 1..448: mol_type = other DNA
atgcttactggacaagaggaaactgatgcacactacatggtcagtgggcttttaggctagtggcatcagtttcccagaatcagacttttgaagatgaatgactttggagaagcaaattaaacatttggccctgagccagcagatcaagcaaatgtctatctttgcgcatgggttgttttttttttttttctttttattctacttggtcagctttgggacgatagtgcagctttgggtgatcttgaaaatcaaatactatcctatactccagctgcttaacttcattttattctttaatgtgtacctgaaagctcctggcaatgctggaaaatttttatcccagaggggtgggggggaggggggaggggaagccagagtccacttttgtcacaattcatttttattaatagaaaataaacacttattccagtttcaaaaaaaaaaaaaa

SEQ ID NO: 256 - DNA, 176 bp, synthetic construct
  misc_feature 1..176: note = Homo sapiens MIPEP 3'-UTR NM_005932
  source 1..176: mol_type = other DNA
aagaaacactctacacctcttaaatcaaggtcatgtagataatgactttgttataaatgctacagctgtgagagcttgtttctgatttcattgttcgcttctgtaattctgaaaaactttaaactggtagaacttggaataaataatttgttttaattaaaaaaaaaaaaaaaaaa

SEQ ID NO: 257 - DNA, 478 bp, synthetic construct
  misc_feature 1..478: note = Homo sapiens LEPR 3'-UTR NM_002303
  source 1..478: mol_type = other DNA
tttcactgaagaaaccttcagatttgtgttataatgggtaatataaagtgtaatagattatagttgtgggtgggagagagaaaagaaaccagagtcaaatttgaaaataattgttccaaatgaatgttgtctgtttgttctctcttagtaacatagacaaaaaatttgagaaagccttcataagcctaccaatgtagacacgctcttctattttattcccaagctctagtgggaaggtcccttgtttccagctagaaataagcccaacagacaccatcttttgtgagatgtaattgttttttcagagggcgtgttgttttacctcaagtttttgttttgtaccaacacacacacacacacacattcttaacacatgtccttgtgtgttttgagagtatattatgtatttatattttgtgctatcagactgtaggatttgaagtaggactttcctaaatgtttaagataaacagaattc

SEQ ID NO: 258 - DNA, 64 bp, synthetic construct
  misc_feature 1..64: note = Homo sapiens LEPR 3'-UTR NM_001198688
  source 1..64: mol_type = other DNA
gaaatgcttgtagactacgtcctacctcgctgccgcacctgctctccctgaggtgtgcacaatg

SEQ ID NO: 259 - DNA, 28 bp, synthetic construct
  misc_feature 1..28: note = Homo sapiens C2orf76 3'-UTR NM_001017927
  source 1..28: mol_type = other DNA
aaacatctcgagggcttcctttttgcat

SEQ ID NO: 260 - DNA, 244 bp, synthetic construct
  misc_feature 1..244: note = Homo sapiens C2orf76 3'-UTR C2orf76-001 ENST00000409466
  source 1..244: mol_type = other DNA
aaacatctcgagggcttcctttttgcatacctgtattaagctctttattccactgctgaatttttgaaattgacaaacaaatcttaaaaaattaatcccaggctatactctttgagctaaaatctggttatttctttctcttcaggtctttccttctctctttctttttctttgttgttgtaaaataatatattatgagaaaaacatttgatctttttaaagggaaataaattgttattaaaaa

SEQ ID NO: 261 - DNA, 267 bp, synthetic construct
  misc_feature 1..267: note = Homo sapiens ABCA6 3'-UTR NM_080284.2
  source 1..267: mol_type = other DNA
aacctcaaacctagtaattttttgttgatctcctataaactcatgttttatgtaataattaatagtatgtttaattttaaagatcatttaaaattaacatcaggtatattttgtaaatttagttaacaaatacataaattttaaaattattcttcctctcaaacataggggtgatagcaaacctgtgataaaggcaatacaaaatattagtaaagtcacccaaagagtcaggcactgggtattgtggaaataaaactatataaactt

SEQ ID NO: 262 - DNA, 45 bp, synthetic construct
  misc_feature 1..45: note = Homo sapiens LY96 3'-UTR NM_015364.4
  source 1..45: mol_type = other DNA
aataaattgagtatttaaaaaaaaaaaaaaaaaaaaaaaaaaaaa

SEQ ID NO: 263 - DNA, 755 bp, synthetic construct
  misc_feature 1..755: note = Homo sapiens CROT 3'-UTR NM_ 001243745.1
  source 1..755: mol_type = other DNA
tgatgatgtttaaagaatgataaataaaaagtgcatagtttttatttttaaattattgctgtaaaaatttttacagttattattgttattttcataatccaaaagaaggaatgaatcacttaactttgggagttttcagtgggtggattcgggaacttgttaaaatgcagatttgctgggataagtgattctgattcacatggctggaatgaggcccagagattcttattttaacaatcacttcatgtggtttggctgcaggtaatctgtagaccatgctgaaggaaaacattttgtccaggtgactagcttgaaaaatcagaaacactaaaatagacatgtcacataggtggcatagaaatattttcgtagtacaatggagaaagggaatcattaaaaatcagagtggagaatggttatgtatattgtatatttcagttagataaattgaggaagctagtataataattattgaaggtctcaataattttccacaaaattctttaacttcttcagctcaaccatttctgtacttctctactatgaatcagaggatgaggttgtataattcaaaagcattgccttagtctagaaataattattgtacctatcatttagttttagaaataaaaagcaagctgattttttttgatgaaccatttatatctgtgatggaataataaaatttcacacttccggattcctttgttctcaattttgagccttgagttgttttaattaaagaggggtaaagg

SEQ ID NO: 264 - DNA, 911 bp, synthetic construct
  misc_feature 1..911: note = Homo sapiens ENPP5 3'-UTR ENPP5-002 ENST00000230565
  source 1..911: mol_type = other DNA
tgttactttgaagtggatttgcatattgaagtggagattccataattatgtcagtgtttaaaggtttcaaattctgggaaaccagttccaaacatttgcagaaaccattaagcagttacatatttaggtatacacacacacacacacacacatacacacacacggaccaaaatacttacacctgcaaaggaataaagatgtgagagtatgtctccattgttcactgtagcatagggatagataagatcctgctttatttggacttggcgcagataatgtatatatttagcaactttgcactatgtaaagtaccttatgtattgcactttaaatttctctcctgatgggtactttaatttgaaatgcactttatgcacagttatgtcttataacttgattgaaaatgacaactttttgcacccatgtcacagaatacttgttacgcattgttcaaactgaaggaaatttctaataatcccgaataatgaacgtagaaatctatctccataaattgagagaagaagaaggtgataagtgttgaaaattaaatgtgataacctttgaaccttgaattttggagatgtattcccaacagcagaatgcaactgtgggcatttcttgtcttatttctttccagagaacgtggttttcatttatttttccctcaaaagagagtcaaatactgacagattcgttctaaatatattgtttctgtcataaaattattgtgatttcctgatgagtcatattactgtgattttcataataatgaagacaccatgaatatactttttttctatatagttcagcaatggcctgaatagaagcaaccaggcaccatctcagcaatgttttctcttgtttgtaattatttgctcctttgaaaattaaatcactattaattacattaa

SEQ ID NO: 265 - DNA, 300 bp, synthetic construct
  misc_feature 1..300: note = Homo sapiens SERPINB7 3'-UTR SERPINB7-203 ENST00000546027
  source 1..300: mol_type = other DNA
aaatccaattggtttctgttatagcagtccccacaacatcaaagaaccaccacaagtcaatagatttgagtttaattggaaaaatgtggtgtttcctttgagtttatttcttcctaacattggtcagcagatgacactggtgacttgacccttcctagacacctggttgattgtcctgatccctgctcttagcattctaccaccatgtgtctcacccatttctaatttcattgtctttcttcccacgctcatttctatcattctcccccatgacccgtctggaaattatggagagtgctc

SEQ ID NO: 266 - DNA, 509 bp, synthetic construct
  misc_feature 1..509: note = Homo sapiens TCP11L2 3'-UTR NM_152772
  source 1..509: mol_type = other DNA
agaagaactgacattggacgagagattggaaatccagtactttggtatccagtccacttccattgatggcattagagatccagcacattctcagtactgtggtgcagtattagcccaaatctgtgtaatgggtaatattagcattacagaagacacacacatcacatagaccctcagaagacgtaaacatcacatagaccctatttgtgcatcattttcaagtttaaaacagatatttgtaatgaacagaaaacaatttgtaattaattatattacctatataatacttgtaaatgttttcttaaccatttatatttggcttatgacatttaacccctaaggagttgtttttctcacttgttattatcaaacctaatggtttttaattttggtacaactccttaaagggttgaaggttgtgacaataactgagggaactgatgttctgaataaatgatgtgaagtaaacacaattgtatttgaaaaaaaaaaaaaaaaaaaaaaaaaaa

SEQ ID NO: 267 - DNA, 84 bp, synthetic construct
  misc_feature 1..84: note = Homo sapiens IRAK1BP1 3'-UTR NM_001010844
  source 1..84: mol_type = other DNA
aattccaaacaaattatattgtacttgtatctttttacctatttttatactttttataatgtttacgtttgtcctgaatatata

SEQ ID NO: 268 - DNA, 338 bp, synthetic construct
  misc_feature 1..338: note = Homo sapiens CDKL2 3'-UTR CDKL2-002 ENST00000307465
  source 1..338: mol_type = other DNA
gaaccattttggttctgaactggatgatgctcttgcacttgagatgacatcttcttgcagcaagagtgctgatatcccaagaggagagattcatggttttgatcatttccttctgaactgcctgcattttctgaggaaggccttctagaagaaggaaagacaaagacttccaaatgtttcaaaggaagattgaacaaatggccctccccaactgttatcccattacctttcacgtccaccgatgctatttcaagacatatccagtggaataacagtgatatggttcttgttacatgaatgtgtatttactgttaggagattgtatattttaagttacc

SEQ ID NO: 269 - DNA, 367 bp, synthetic construct
  misc_feature 1..367: note = Homo sapiens GHR 3'-UTR GHR-202 ENST00000537449
  source 1..367: mol_type = other DNA
cctttctttggtttcccaagagctacgtatttaatagcaaagaattgactggggcaataacgtttaagccaaaacaatgtttaaaccttttttgggggagtgacaggatggggtatggattctaaaatgccttttcccaaaatgttgaaatatgatgttaaaaaaataagaagaatgcttaatcagatagatattcctattgtgcaatgtaaatattttaaagaattgtgtcagactgtttagtagcagtgattgtcttaatattgtgggtgttaatttttgatactaagcattgaatggctatgtttttaatgtatagtaaatcacgctttttgaaaaagcgaaaaaatcaggtggcttttgcggt

SEQ ID NO: 270 - DNA, 406 bp, synthetic construct
  misc_feature 1..406: note = Homo sapiens KIAA1107 3'-UTR NM_015237
  source 1..406: mol_type = other DNA
gtgttaacattttggaaaaatttatgccactcctttattttttgatgcctatattatatccaaatgataattgcattagccggatataaactttctttaatattgagtctttccaatttaatgaggtaaacatagtttatttattaatatatcacatatagaaaaatgtttttctaaagtttttgagcatgttttctctaattattagagaaattagaagacttataaggaaaccctagcttcagttttcctttcctagctgatgatttgttcacttaatcattattcaagaatttaaaatgtgaatgcagaagtagatcagtccctttactttttgctctgcatagggtaacatagtaatttaacaataaaaacttaccgtgcttgtgtccaaaaaaaaaaaaaa

SEQ ID NO: 271 - DNA, 301 bp, synthetic construct
  misc_feature 1..301: note = Homo sapiens RPS6KA6 3'-UTR RPS6KA6-001 ENST00000262752
  source 1..301: mol_type = other DNA
gatttgtggtgttcctaggccaaactggatgaagatgaaattaaatgtgtggcttttttcctattcttatcaaaggcatcgttgtctgctaaattacttgaatattaagtaatattaaatccccatttttaggggaagtgagatttaaaaaaccattcacaggtccacaatattcatactatgtgtttgcagtagtgttcaagtgtttatttaagcatataattggtgtccaccaggtcctcacaacttctctgcacacaagcttctaaaattcctttcaaataaagttactttaatattt

SEQ ID NO: 272 - DNA, 777 bp, synthetic construct
  misc_feature 1..777: note = Homo sapiens CLGN 3'-UTR NM_004362, NM_001130675
  source 1..777: mol_type = other DNA
actagattgaaatatttttaattcccgagagggatgtttggcattgtaaaaatcagcatgccagacctgaactttaatcagtctgcacatcctgtttctaatatctagcaacattatattctttcagacatttattttagtccttcatttcagaggaaaaagaagcaactttgaagttacctcatctttgaatttagaataaaagtggcacattacatatcggatctaagagattaataccattagaagttacacagttttagttgtttggagatagttttggtttgtacagaacaaaataatatgtagcagcttcattgctattggaaaaatcagttattggaatttccacttaaatggctatacaacaatataactggtagttctataataaaaatgagcatatgttctgttgtgaagagctaaatgcaataaagtttctgtatggttgtttgattctatcaacaattgaaagtgttgtatatgacccacatttacctagtttgtgtcaaattatagttacagtgagttgtttgcttaaattatagattcctttaaggacatgccttgttcataaaatcactggattatattgcagcatattttacatttgaatacaaggataatgggttttatcaaaacaaaatgatgtacagattttttttcaagtttttatagttgctttatgccagagtggtttaccccattcacaaaatttcttatgcatacattgctattgaaaataaaatttaaatattttttcatcctgaaaaaaaa

SEQ ID NO: 273 - DNA, 466 bp, synthetic construct
  misc_feature 1..466: note = Homo sapiens CLGN-202 3'-UTR NM_004362, NM_001130675 ENST00000325617
  source 1..466: mol_type = other DNA
actagattgaaatatttttaattcccgagagggatgtttggcattgtaaaaatcagcatgccagacctgaactttaatcagtctgcacatcctgtttctaatatctagcaacattatattctttcagacatttattttagtccttcatttcagaggaaaaagaagcaactttgaagttacctcatctttgaatttagaataaaagtggcacattacatatcggatctaagagattaataccattagaagttacacagttttagttgtttggagatagttttggtttgtacagaacaaaataatatgtagcagcttcattgctattggaaaaatcagttattggaatttccacttaaatggctatacaacaatataactggtagttctataataaaaatgagcatatgttctgttgtgaagagctaaatgcaataaagtttctgtatggttgtttgattctatcaac

SEQ ID NO: 274 - DNA, 423 bp, synthetic construct
  misc_feature 1..423: note = Homo sapiens TMEM45A 3'-UTR NM_018004
  source 1..423: mol_type = other DNA
ctttgatgagcttccagtttttctagataaaccttttcttttttacattgttcttggttttgtttctcgatcttttgtttggagaacagctggctaaggatgactctaagtgtactgtttgcatttccaatttggttaaagtatttgaatttaaatattttctttttagctttgaaaatattttgggtgatactttcattttgcacatcatgcacatcatggtattcaggggctagagtgatttttttccagattatctaaagttggatgcccacactatgaaagaaatatttgttttatttgccttatagatatgctcaaggttactgggcttgctactatttgtaactccttgaccatggaattatacttgtttatcttgttgctgcaatgagaaataaatgaatgtatgtattttggtgc

SEQ ID NO: 275 - DNA, 152 bp, synthetic construct
  misc_feature 1..152: note = Homo sapiens TBC1D8B 3'-UTR TBC1D8B-007 ENST00000276175
  source 1..152: mol_type = other DNA
atccctaggaattgcctatcatagacaagtttactaacattcctgtagctgtcagtttgattcctgtgagtagggctcagggatttatcttgttaccaatgtgtctgaaggccaaaatatatatccagaagcacaatgcatcattcctttgt

SEQ ID NO: 276 - DNA, 81 bp, synthetic construct
  misc_feature 1..81: note = Homo sapiens ACP6 3'-UTR NM_016361
  source 1..81: mol_type = other DNA
ctgatttataaaagcaggatgtgttgattttaaaataaagtgcctttatacaatgccaaaaaaaaaaaaaaaaaaaaaaaa

SEQ ID NO: 277 - DNA, 111 bp, synthetic construct
  misc_feature 1..111: note = Homo sapiens RP6-213H19.1 3'-UTR MST4-003 (RBM4B-003 ENST00000496850)
  source 1..111: mol_type = other DNA
gaaacttattattggcttctgtttcatatggacccagagagccccaccaaacctacgtcaagattaacaatgcttaacccatgagctccatgtgccttttggatctttgca

SEQ ID NO: 278 - DNA, 138 bp, synthetic construct
  misc_feature 1..138: note = Homo sapiens SNRPN 3'-UTR NM_022807
  source 1..138: mol_type = other DNA
catactgttgatccatctcagtcactttttcccctgcaatgcgtcttgtgaaattgtgtagagtgtttgtgagctttttgttccctcattctgcattaataatagctaataataaatgcatagagcaattaaactgtg

SEQ ID NO: 279 - DNA, 425 bp, synthetic construct
  misc_feature 1..425: note = Homo sapiens GLRB 3'-UTR GLRB-005 ENST00000512619
  source 1..425: mol_type = other DNA
gatctaatgacttcagcattgttggaagcttaccaagagattttgaactatccaattatgactgctatggaaaacccattgaagttaacaacggacttgggaaatctcaggctaagaacaacaagaagcctccccctgcgaaacctgttattccaacagcagcaaagcgaattgatctttatgcaagagcattgtttcctttctgcttcttgttcttcaatgttatatattggtctatatatttatgataaatcttttccatttgtacaaaataaaattccatttcattgtgacctactcctttcataaatgccaatctgtgagaacttttgaattttcatagcaacattgcattttggatgccatttgattgtaataaaactgtggcaccttaattttgaatggcagcatgatcatgtaatatc

SEQ ID NO: 280 - DNA, 651 bp, synthetic construct
  misc_feature 1..651: note = Homo sapiens HERC6 3'-UTR NM_017912
  source 1..651: mol_type = other DNA
tcacctctgagagactcagggtgggctttctcacacttggatccttctgttcttccttacacctaaataatacaagagattaatgaatagtggttagaagtagttgagggagagattgggggaatggggagatgatgatgatggtcaaagggtgcaaaatctcacacaagactgaggcaggagaatagggtacagagatagggatctaaggatgacttggacacactccctggcactgaagagtctgaacactggcctgtgattggtccattccaggaccttcatttgcataaggtatcaaaccacatcagcctctgattggccatgggccagacctgcactctggccaatgattggttcattccaggacattcatttgcataaggagtcaaaccacaccagtcttggattggctgtgagccaattcacctcagtctctaattggctgtgagtcagtctttcatttacatagggtgtaaccatcaagaaacctctacagggtacttaagccccagaagattttgctaccagggctcttgagccacttgctctagcccactcccaccctgtggaatgtactttcacttttgctgcttcactgccttgtgctccaataaatccactccttcaccacccaaaaaaaaaaaaaaa

SEQ ID NO: 281 - DNA, 264 bp, synthetic construct
  misc_feature 1..264: note = Homo sapiens CFH 3'-UTR NM_000186
  source 1..264: mol_type = other DNA
aatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctcttcttaaaagcaccatattaaatcctggaaaactaaaaaaaaaaaaaaaaaaaaaaaaaaaaaaaaaaaaaaaaaaaaaaaaaaaaaaaaaaaaaaaaaaaaaaaaaaa

SEQ ID NO: 282 - DNA, 401 bp, synthetic construct
  misc_feature 1..401: note = Homo sapiens GALC 3'-UTR GALC-002 ENST00000393569
  source 1..401: mol_type = other DNA
tacttaacagggcatcatagaatactctggattttcttcccttctttttggttttggttcagagccaattcttgtttcattggaacagtatatgaggcttttgagactaaaaataatgaagagtaaaaggggagagaaatttatttttaatttaccctgtggaagattttattagaattaattccaaggggaaaactggtgaatctttaacattacctggtgtgttccctaacattcaaactgtgcattggccatacccttaggagtggtttgagtagtacagacctcgaagccttgctgctaacactgaggtagctctcttcatcttatttgcaagcggtcctgtagatggcagtaacttgatcatcactgagatgtatttatgcatgctgaccgtgtgt

SEQ ID NO: 283 - DNA, 51 bp, synthetic construct
  misc_feature 1..51: note = Homo sapiens GALC 3'-UTR GALC-005 ENST00000393568
  source 1..51: mol_type = other DNA
tacttaacagggcatcatagaatactctggattttcttcccttctttttgg

SEQ ID NO: 284 - DNA, 360 bp, synthetic construct
  misc_feature 1..360: note = Homo sapiens PDE1A 3'-UTR NM_001003683.2
  source 1..360: mol_type = other DNA
acacctttaagtaaaacctcgtgcatggtggcagctctaatttgaccaaaagacttggagattttgattatgcttgctggaaatctaccctgtcctgtgtgagacaggaaatctatttttgcagattgctcaataagcatcatgagccacataaataacagctgtaaactccttaattcaccgggctcaactgctaccgaacagattcatctagtggctacatcagcaccttgtgctttcagatatctgtttcaatggcattttgtggcatttgtctttaccgagtgccaataaattttctttgagcagctaattgctaattttgtcatttctacaataaagcttggtccacctgttttc

SEQ ID NO: 285 - DNA, 308 bp, synthetic construct
  misc_feature 1..308: note = Homo sapiens PDE1A 3'-UTR PDE1A-003 ENST00000410103
  source 1..308: mol_type = other DNA
acacctttaagtaaaacctcgtgcatggtggcagctctaatttgaccaaaagacttggagattttgattatgcttgctggaaatctaccctgtcctgtgtgagacaggaaatctatttttgcagattgctcaataagcatcatgagccacataaataacagctgtaaactccttaattcaccgggctcaactgctaccgaacagattcatctagtggctacatcagcaccttgtgctttcagatatctgtttcaatggcattttgtggcatttgtctttaccgagtgccaataaattttctttgagca

SEQ ID NO: 286 - DNA, 855 bp, synthetic construct
  misc_feature 1..855: note = Homo sapiens GSTM5 3'-UTR NM_000851
  source 1..855: mol_type = other DNA
ggcccagtgatgccagaagatgggagggaggagccaaccttgctgcctgcgaccctggaggacagcctgactccctggacctgccttcttcctttttccttctttctactctcttctcttccccaaggcctcattggcttcctttcttctaacatcatccctccccgcatcgaggctctttaaagcttcagctccccactgtcctccatcaaagtccccctcctaacgtcttcctttccctgcactaacgccaacctgactgcttttcctgtcagtgcttttctcttctttgagaagccagactgatctctgagctccctagcactgtcctcaaagaccatctgtatgccctgctccctttgctgggtccctaccccagctccgtgtgatgcccagtaaagcctgaaccatgcctgccatgtcttgtcttattccctgaggctcccttgactcaggactgtgctcgaattgtgggtggttttttgtcttctgttgtccacagccagagcttagtggatgggtgtgtgtgtgtgtgtgttgggggtggtgatcaggcaggttcataaatttccttggtcatttctgccctctagccacatccctctgttcctcactgtggggattactacagaaaggtgctctgtgccaagttcctcactcattcgcgctcctgtaggccgtctagaactggcatggttcaaagaggggctaggctgatggggaagggggctgagcagctcccaggcagactgccttctttcaccctgtcctgatagacttccctgatctagatatccttcgtcatgacacttctcaataaaacgtatcccaccgtattgtaaaaaaaaaaaaaaa

SEQ ID NO: 287 - DNA, 419 bp, synthetic construct
  misc_feature 1..419: note = Homo sapiens CADPS2 3'-UTR CADPS2-002 ENST00000412584
  source 1..419: mol_type = other DNA
tatcacacagctttgcagaaggaaggaagaccttgatcgacattgttttttatttttttaaccttgtccttgtaattacattcattgtttgttttggccaaataaaaatgcttgtatttctttaaaaagtaagcctgaatgtagagtaaaaggggaaatgccaagattttggggtttttttgtttcctttttttgtttgtttgtttgtttgtttttttggagaagagcatcctcttttgtgtagtttgacctaaaaatgaaccttggctctgcttgtgatcagaacatgaacttttttttttaaagaagatttgagcatttttctgtaatcacatcaaaatgatgttttctgtgtaaagcgagatacatatttctcataatgcagcattgtgagaagtcagttcggaccactgcaccaa

SEQ ID NO: 288 - DNA, 162 bp, synthetic construct
  misc_feature 1..162: note = Homo sapiens CADPS2 3'-UTR CADPS2-001 ENST00000449022
  source 1..162: mol_type = other DNA
tatcacacagctttgcagaaggaaggaagaccttgatcgacattgttttttatttttttaaccttgtccttgtaattacattcattgtttgttttggccaaataaaaatgcttgtatttctttaaaaagtaagcctgaatgtagagtaaaaggggaaatgcc

SEQ ID NO: 289 - DNA, 247 bp, synthetic construct
  misc_feature 1..247: note = Homo sapiens AASS 3'-UTR AASS-001 ENST00000417368
  source 1..247: mol_type = other DNA
ttgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaatacgtatgtgaaagtgatga

SEQ ID NO: 290 - DNA, 695 bp, synthetic construct
  misc_feature 1..695: note = Homo sapiens TRIM6-TRIM34 3'-UTR NM_001003819
  source 1..695: mol_type = other DNA
attttctcatttcttcacctacaaccctttgtcttgacttatctcctgcaactgactcatctgcaacattcacaccattgcttccttgtggtttcccttctttagaacttttactcatccttgagatgtatggtgtatttggcttgagttatgagagatgcttatttattcatttactctttttcatattttcagagaaagttacctaatccctcctaaagacacagcagtatgggtataacatccttgccttcccatttatccatgtttcactttatcactgatatgaagaggcccaaagcctgttagccaccatccatgctacctaggtagtccataggaaccacccccatgaccaccaccaacatcaactaaaggttcttggagggtatgtcagtgtgttgctcaggataccccaggtacatcaaggaatcaaggagaggaaaatatgagcaatatgtgtattcagagtgaagattttatgtccagagtatttgagctcaaaccttgcctgttgttttctaatcatgatgaatactttctcagtttctttttcctgaaatataaattgggatttaagactgtacctaactattaagatcactgtgtaaaactaagtgtctctaaatgtaatgcatcgatttagtgtctggaacataataaatatttgctctcatgattgctaaaaaaaaaaa

SEQ ID NO: 291 - DNA, 918 bp, synthetic construct
  misc_feature 1..918: note = Homo sapiens SEPP1 3'-UTR NM_005410
  source 1..918: mol_type = other DNA
atatttaaaataggacatactccccaatttagtctagacacaatttcatttccagcatttttataaactaccaaattagtgaaccaaaaatagaaattagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataattagcagtttagaatggaggaagaacaacaaagacatgctttccatttttttctttacttatctctcaaaacaatattactttgtcttttcaatcttctacttttaactaataaaataagtggattttgtattttaagatccagaaatacttaacacgtgaatattttgctaaaaaagcatatataactattttaaatatccatttatcttttgtatatctaagactcatcctgatttttactatcacacatgaataaagcctttgtatctttctttctctaatgttgtatcatactcttctaaaacttgagtggctgtcttaaaagatataaggggaaagataatattgtctgtctctatattgcttagtaagtatttccatagtcaatgatggtttaataggtaaaccaaaccctataaacctgacctcctttatggttaatactattaagcaagaatgcagtacagaattggatacagtacggatttgtccaaataaattcaataaaaaccttaaagctgaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 292 - DNA, 589 bp, synthetic construct
  misc_feature 1..589: note = Homo sapiens SEPP1 3'-UTR SEPP1-004 ENST00000506577
  source 1..589: mol_type = other DNA
atatttaaaataggacatactccccaatttagtctagacacaatttcatttccagcatttttataaactaccaaattagtgaaccaaaaatagaaattagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataattagcagtttagaatggaggaagaacaacaaagacatgctttccatttttttctttacttatctctcaaaacaatattactttgtcttttcaatcttctacttttaactaataaaataagtggattttgtattttaagatccagaaatacttaacacgtgaatattttgctaaaaaagcatatataactattttaaatatccatttatcttttgtatatctaagactcatcctgatttttactatcacacatgaataaagcctttgtatcttt

SEQ ID NO: 293 - DNA, 60 bp, synthetic construct
  misc_feature 1..60: note = Homo sapiens PDE5A 3'-UTR PDE5A-002 ENST00000264805
  source 1..60: mol_type = other DNA
gtggcctatttcatgcagagttgaagtttacagagatggtgtgttctgcaatatgcctag

SEQ ID NO: 294 - DNA, 422 bp, synthetic construct
  misc_feature 1..422: note = Homo sapiens SATB1 3'-UTR SATB1-004 ENST00000417717
  source 1..422: mol_type = other DNA
gataaaagtatttgtttcgttcaacagtgccactggtatttactaacaaaatgaaaagtccaccttgtcttctctcagaaaacctttgttgttcattgtttggccaatgaatcttcaaaaacttgcacaaacagaaaagttggaaaaggataatacagactgcactaaatgttttcctctgttttacaaactgcttggcagccccaggtgaagcatcaaggattgtttggtattaaaatttgtgttcacgggatgcaccaaagtgtgtaccccgtaagcatgaaaccagtgttttttgttttttttttagttcttattccggagcctcaaacaagcattataccttctgtgattatgatttcctctcctataattatttctgtagcactccacactgatctttggaaacttgccccttattt

SEQ ID NO: 295 - DNA, 981 bp, synthetic construct
  misc_feature 1..981: note = Homo sapiens CCPG1 3'-UTR CCPG1-002 ENST00000442196
  source 1..981: mol_type = other DNA
ttcacaattgagttaaattagacaactgtaagagaaaaatttatgctttgtataatgtttggtattgaaactaatgaaattaccaagatgacaatgtcttttcttttgtttctaagtatcagtttgataactttatattattcctcagaagcattagttaaaagtctactaacctgcattttcctgtagtttagcttcgttgaattttttttgacactggaaatgttcaactgtagttttattaaggaagccaggcatgcaacagattttgtgcatgaaatgagacttcctttcagtgtaagagcttaaagcaagctcagtcatacatgacaaagtgtaattaacactgatgtttgtgttaaatttgcagcagagcttgagaaaagtacattgttctggaatttcatcattaacattttataatcttacactcacttcttgtctttttgtgggttcaagagccctctgacttgtgaagaatttgctgccctcttaagagcttgctgacttgttttcttgtgaaattttttgcacatctgaatatcgtggaagaaacaataaaactacaccatgaggaaaactaaaggtctttatttaaaatctggcattgtattaacatgtaattttatactatgtggtattttatacatttcctcagtagtgatatttggtaaagcagttcatacagcttttttctaagttccatgaatcttacccagtgtttaccgaagtatttaagcagcatctgaatatttccacccagcaatgttaatttatctaggaaagttcagaatttcatcttcatgttgaatttcccttttaacttccgttcatagacatatatgtgacttccaattcgaccctctggcaagtgagtgtggaagaaaacagcagttcttttataattgcttgaaattaggaaagcgcttatttcctagaagcaaataaatgtttaagtaaataaaggctacattttgctga

SEQ ID NO: 296 - DNA, 575 bp, synthetic construct
  misc_feature 1..575: note = Homo sapiens CCPG1 3'-UTR CCPG1-004 ENST00000425574
  source 1..575: mol_type = other DNA
ttcacaattgagttaaattagacaactgtaagagaaaaatttatgctttgtataatgtttggtattgaaactaatgaaattaccaagatgacaatgtcttttcttttgtttctaagtatcagtttgataactttatattattcctcagaagcattagttaaaagtctactaacctgcattttcctgtagtttagcttcgttgaattttttttgacactggaaatgttcaactgtagttttattaaggaagccaggcatgcaacagattttgtgcatgaaatgagacttcctttcagtgtaagagcttaaagcaagctcagtcatacatgacaaagtgtaattaacactgatgtttgtgttaaatttgcagcagagcttgagaaaagtacattgttctggaatttcatcattaacattttataatcttacactcacttcttgtctttttgtgggttcaagagccctctgacttgtgaagaatttgctgccctcttaagagcttgctgacttgttttcttgtgaaattttttgcacatctgaatatcgtggaagaaacaataaaactacaccatgag

SEQ ID NO: 297 - DNA, 230 bp, synthetic construct
  misc_feature 1..230: note = Homo sapiens CNTN1 3'-UTR CNTN1-002 ENST00000348761
  source 1..230: mol_type = other DNA
atgtgttgtgacagctgctgttcccatcccagctcagaagacacccttcaaccctgggatgaccacaattccttccaatttctgcggctccatcctaagccaaataaattatactttaacaaactattcaactgatttacaacacacatgatgactgaggcattcgggaaccccttcatccaaaagaataaacttttaaatggatataaatgatttttaactcgttccaa

SEQ ID NO: 298 - DNA, 922 bp, synthetic construct; identical to SEQ ID NO: 21
  misc_feature 1..922: note = Homo sapiens CNTN1 3'-UTR CNTN1-004 ENST00000547849
  source 1..922: mol_type = other DNA

SEQ ID NO: 299 - DNA, 928 bp, synthetic construct; identical to SEQ ID NO: 22
  misc_feature 1..928: note = Homo sapiens CNTN1 3'-UTR CNTN1-004 ENST00000547849 +T at pos. 30bp, mutations G727bpT, A840bpG
  source 1..928: mol_type = other DNA

SEQ ID NO: 300 - DNA, 734 bp, synthetic construct
  misc_feature 1..734: note = Homo sapiens LMBRD2 3'-UTR
  source 1..734: mol_type = other DNA
agtctgaaaaagtttgtgggaccactaaccaaggtcaacacatcagttcagtcttgatgaacatctgtgtaccctagaatttcctctatacacagtgaaaagtgtcaagataacaaaaaaggcactgagaattaattatatcttaggaataatagtttaatgtgcattgaatagagtatcacctttttcaacaagatttattacatatcatttcctaagcatctgccttagaaatacagttacagtggaaggactttaagaaagatcaacatatgttaagaacatgcagttcagtttgtttcagattaattttttttcaagagagttattttaaagattcaaggaagccataagtcatactaaataatattatatacagttttgttattgtgacttacatttttgttacttctaaaaagtatattcaacctgtatttcccaaagaaatgtaagtgaatggagacctcaaataataactgtattcataaaactcgtgtcttaaaacaaggcttacttactagacataactgaatgtaaaaagtgctttttcaaatctgtttgcaaactcgtgggggatttttgcatgtataagattaagattatacttcaagtgatgcgtgtctgtgtatttagcatgtgtactataatcaggtgatatagtattccttcagtctttgtagtaactggatttttttatgcttctggtattgctttataaaagattttcatttcag

SEQ ID NO: 301 - DNA, 241 bp, synthetic construct
  misc_feature 1..241: note = Homo sapiens TLR3 3'-UTR NM_003265
  source 1..241: mol_type = other DNA
atttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgtaaaaaaaaaaaaaaaaaa

SEQ ID NO: 302 - DNA, 527 bp, synthetic construct
  misc_feature 1..527: note = Homo sapiens BCAT1 3'-UTR BCAT1-002 ENST00000342945
  source 1..527: mol_type = other DNA
atggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttacctagaaaaacattaatgtaagccatataacatgggattttcctcaatgattttagtgcctccttttgtacttcactcagatactaaatagtagtttattctttaatataagttacattctgctcctcaaacaaatgcaattttttgtgtgtgtttgaaagctaatttgagaaaatttcataggttacatttcctgcagcctatctttatccacagaaagtgttttcttttttttaaatcaagacttttaaaactggatttcctcccatcactgttttttgaaggtcctccaagtccgtgttaa

SEQ ID NO: 303 - DNA, 199 bp, synthetic construct
  misc_feature 1..199: note = Homo sapiens BCAT1 3'-UTR
  source 1..199: mol_type = other DNA
atggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttg

SEQ ID NO: 304 - DNA, 716 bp, synthetic construct
  misc_feature 1..716: note = Homo sapiens TOM1L1 3'-UTR TOM1L1-001 ENST00000575882
  source 1..716: mol_type = other DNA
gaagaaagtggatgatcagctcactaccacatcaaaggtgccaactctctaaaacgtagactctgtgcagctttgaagcctggaagacaatacctaccaacatgtcaaagccatggtggcacatttctgctataatgaagattaaatagaataacagttccaggataacactgattcctgacaacagcgtgagatttcaacagaacttgtttggaacaaatactcacttaaaacttcagcagaagaaaaattacttagtccttaggccaaccaatttaactgcagtgtcatgtttcacaggccttcctacatttagaaatcgtcacacagctgtgataagagtagattattttactatgaaataattctgaatagatgaaagcataaaatgtgagaaactgaatgtattattcaggaagaatactgagtgccttcatttaactaaagttgaatgtaaaagtcaatttgcacttctttataatcctctggtttagaattataaattgttaaaaccttgataattgtcatttaattatatttcaggtgtcctgaacaggtcactagactctacattgggcagcctttaaatatgattctttgtaatgctaaatagcctttttttctctttttactgcaacttaatatttctatttagaacacagaaaatgaaaatatttagaataagttgtacatttgatgacaaataaatcactatt

SEQ ID NO: 305 - DNA, 804 bp, synthetic construct
  misc_feature 1..804: note = Homo sapiens SLC35A1 3'-UTR SLC35A1-201 ENST00000369556
  source 1..804: mol_type = other DNA
ttttagcctcacgtgagactccttttaagactaaaccatttgcattaaactagagccttaagtcaatctcagaaggtagcataaacaaataaaaattaactgtatggcatgatcagtgcggttatgtggaaacaacaacaaacaaacgaagctatctgagtgaactgctaatacagaaacttaatgtagacctgtttggggtctactattgttttagaatgaaggaattgtattattgtgtgtatatataatttgtaaataaaaagtatggagatgatacggtgttaaaaaaaatcatggtaaggctacaatactcaagtaacaaggtttgggacaatgtctaagggttaaagtgccaaagccatttctgtactaactgttctcttgttccggtaccggggagaaggatgacccctccttattctccaattcatgtacagtattttgtcctagcagcataaagacctagctcttttcttacaagaggcagaaacaagacaggctagttcataaacaaactgtgtaacttctcaaaatgaatctatttcataactcggacaatttctgggtggtgactgagtacccctttagtgagtacccctttagtgctatatttgtgccattcattatctggttcatatttcttttctgttagatgatacacatttcttcaaaaaaatttctaatgtcacttttgtacttttttaaataaagtatgtttaactgttgggctctcaataatttgtgaaatttcagtgttttctataatgttaatggggaaattcagcaataaactttatttgt

SEQ ID NO: 306 - DNA, 332 bp, synthetic construct
  misc_feature 1..332: note = Homo sapiens GLYATL2 3'-UTR GLYATL2-003 ENST00000532258
  source 1..332: mol_type = other DNA
ttgattccactgtccatttcaaatctttcttatcagtaaaaaaacattaattcaaacacaagcattgtgatctacattagcacaaaatgcaactgattatctaggatctgtgtattacttaagctcacccttaacagttttaccttccttctcctctgtattcttacagaaaattagaagctcaattttatggtctcataatttcctttatgacagacatctcagaattaaaatcacccaaagccaatcattagtgccaagataaccctttaacggcaacactttcttaaatgaagactatttctttcatgaaaaaattcacttttatgact

SEQ ID NO: 307 - DNA, 260 bp, synthetic construct
  misc_feature 1..260: note = Homo sapiens STAT4 3'-UTR STAT4-002 ENST00000392320
  source 1..260: mol_type = other DNA
caggataaactctgacgcaccaagaaaggaagcaaatgaaaaagtttaaagactgttctttgcccaataaccacattttatttcttcagctttgtaaataccaggttctaggaaatgtttgacatctgaagctctcttcacactcccgtggcactcctcaattgggagtgttgtgactgaaatgcttgaaaccaaagcttcagataaacttgcaagataagacaactttaagaaaccagtgttaataacaatattaacag

SEQ ID NO: 308 - DNA, 270 bp, synthetic construct
  misc_feature 1..270: note = Homo sapiens GULP1 3'-UTR GULP1-002 ENST00000409609
  source 1..270: mol_type = other DNA
catcaagaacaagaaatcctgattcatgttaaatgtgtttgtatacacatgtcatttattattattactttaagataggtattattcatgtgtcaatgtttttgaatattttaatattttgaaaattttctcagttaaatttcctcaccttcactattgatctgtaatttttattttaaaaacagcttactgtaaagtagatcatacttttatgttcctttctgtttctactgtagatgaatttgtaattgaaagacatattatacaaat

SEQ ID NO: 309 - DNA, 79 bp, synthetic construct
  misc_feature 1..79: note = Homo sapiens GULP1 3'-UTR GULP1-010 ENST00000409805
  source 1..79: mol_type = other DNA
catcaagaacaagaaatcctgattcatgttaaatgtgtttgtatacacatgtcatttattattattactttaagatagg

SEQ ID NO: 310 - DNA, 256 bp, synthetic construct
  misc_feature 1..256: note = Homo sapiens EHHADH 3'-UTR EHHADH-002 ENST00000456310
  source 1..256: mol_type = other DNA
ttcagtcttccagattatgcctcacatgctagcatcaggtaatgctgactgaatttcagtgaaattaaatcaaaaatccaaagtaagattgttctgaaatacaaagcaaaataaataatcattagaatcttctgtgtaacgactctaatggtcaaatctttaggaatgtgcttcctatgcctctgaatctgtccttatcagataaattcaatgcatgaacttgtgtgaatataataccataatagctaatgaaaga

SEQ ID NO: 311 - DNA, 640 bp, synthetic construct
  misc_feature 1..640: note = Homo sapiens NBEAL1 3'-UTR NM_001114132.1
  source 1..640: mol_type = other DNA
ttgttatttccattttctgttatgattactgaaacctgatttattgctttgtcactttaaccacatctctcaactctctgcaatgttgcaaggcttttatccctgaaaatcatttacagataaccacaatttgctgtggtatataaactaattcttggtctatactaagatgtatttgagaaaatacatttgatttgattttgtggcccattcctaaaggtcattgtatccatttttaaaacaaactaaaatgagaacattaggttcaattttcttattattccaaatgataaaatttaagatttttctaataaaagagtacagataatgggacagttgagagagatggctttaaatacattcttaagtaatcattttcctatttactgaccactgtaatgaaaatatatcaatttatttatggaactcctgattggggataatattttaaaggtatctgttgcacacttggattttcaaaactcggtgaaagttacaagtttgcatggtaagaataaaataagaatattgaaactggtacattagctaattctattactacttagcgtgtttctaatgagaagttactgaaatctattactgtccttaataaaaattgagtagaaaaaagtggaactag

SEQ ID NO: 312 - DNA, 225 bp, synthetic construct
  misc_feature 1..225: note = Homo sapiens KIAA1598 3'-UTR NM_001258299.1
  source 1..225: mol_type = other DNA
tctgaatcagaaaatactgcaactccttcctccttttgtctgccttttgttctccaaaagtaagtggaaattacatttccaagaaaggaaatgaaataattgcaggcccaaggtctgcaaaatatgtgttgaattgacagtgaaaaggatccatgtgttgacagacacagttgttagatgccataaaggcagatgtgaagctcaatttatttctcatcttgcttg

SEQ ID NO: 313 - DNA, 991 bp, synthetic construct
  misc_feature 1..991: note = Homo sapiens HFE 3'-UTR HFE-006 ENST00000317896
  source 1..991: mol_type = other DNA
cacgcagcctgcagactcactgtgggaaggagacaaaactagagactcaaagagggagtgcatttatgagctcttcatgtttcaggagagagttgaacctaaacatagaaattgcctgacgaactccttgattttagccttctctgttcatttcctcaaaaagatttccccatttaggtttctgagttcctgcatgccggtgatccctagctgtgacctctcccctggaactgtctctcatgaacctcaagctgcatctagaggcttccttcatttcctccgtcacctcagagacatacacctatgtcatttcatttcctatttttggaagaggactccttaaatttgggggacttacatgattcattttaacatctgagaaaagctttgaaccctgggacgtggctagtcataaccttaccagatttttacacatgtatctatgcattttctggacccgttcaacttttcctttgaatcctctctctgtgttacccagtaactcatctgtcaccaagccttggggattcttccatctgattgtgatgtgagttgcacagctatgaaggctgtacactgcacgaatggaagaggcacctgtcccagaaaaagcatcatggctatctgtgggtagtatgatgggtgtttttagcaggtaggaggcaaatatcttgaaaggggttgtgaagaggtgttttttctaattggcatgaaggtgtcatacagatttgcaaagtttaatggtgccttcatttgggatgctactctagtattccagacctgaagaatcacaataattttctacctggtctctccttgttctgataatgaaaattatgataaggatgataaaagcacttacttcgtgtccgactcttctgagcacctacttacatgcattactgcatgcacttcttacaataattctatgagataggtactattatccccatttcttttttaaatgaagaaagtgaagtaggccgggcac

SEQ ID NO: 314 - DNA, 761 bp, synthetic construct
  misc_feature 1..761: note = Homo sapiens HFE 3'-UTR HFE-004 ENST00000349999
  source 1..761: mol_type = other DNA
cacgcagcctgcagactcactgtgggaaggagacaaaactagagactcaaagagggagtgcatttatgagctcttcatgtttcaggagagagttgaacctaaacatagaaattgcctgacgaactccttgattttagccttctctgttcatttcctcaaaaagatttccccatttaggtttctgagttcctgcatgccggtgatccctagctgtgacctctcccctggaactgtctctcatgaacctcaagctgcatctagaggcttccttcatttcctccgtcacctcagagacatacacctatgtcatttcatttcctatttttggaagaggactccttaaatttgggggacttacatgattcattttaacatctgagaaaagctttgaaccctgggacgtggctagtcataaccttaccagatttttacacatgtatctatgcattttctggacccgttcaacttttcctttgaatcctctctctgtgttacccagtaactcatctgtcaccaagccttggggattcttccatctgattgtgatgtgagttgcacagctatgaaggctgtacactgcacgaatggaagaggcacctgtcccagaaaaagcatcatggctatctgtgggtagtatgatgggtgtttttagcaggtaggaggcaaatatcttgaaaggggttgtgaagaggtgttttttctaattggcatgaaggtgtcatacagatttgcaaagtttaatggtgccttcatttgggatg

SEQ ID NO: 315 - DNA, 142 bp, synthetic construct
  misc_feature 1..142: note = Homo sapiens HFE 3'-UTR HFE-005 ENST00000397022
  source 1..142: mol_type = other DNA
cacgcagcctgcagactcactgtgggaaggagacaaaactagagactcaaagagggagtgcatttatgagctcttcatgtttcaggagagagttgaacctaaacatagaaattgcctgacgaactccttgattttagccttc

SEQ ID NO: 316 - DNA, 31 bp, synthetic construct
  misc_feature 1..31: note = Homo sapiens HFE 3'-UTR HFE-012 ENST00000336625
  source 1..31: mol_type = other DNA
cacgcagcctgcagactcactgtgggaagga

SEQ ID NO: 317 - DNA, 394 bp, synthetic construct
  misc_feature 1..394: note = Homo sapiens KIAA1324L 3'-UTR KIAA1324L-005 ENST00000416314
  source 1..394: mol_type = other DNA
agagacagtgctgtagccttgagactaatgaacaaagaaacctgctctagttttacaggaccatattttagggtctgtcctcatacctgtcacattggtgatctcacagaggagggccatgccgctgaaaagggaaggagattgaaacatttgattgccttatcacatggtcaagtaccttgccaaataaaggaaagcaaatgatttgggtctcaactgaagatgaagctcaactcaggaagagatttatctgtatatacacataactgaaaaccaagtttaagcccaccaatgcactgctgatgcatgccatataattaatgggtaacttttattctttatgatgtctacataacaagtgtgatttggaaggcacatgtgagcatatgcatta

SEQ ID NO: 318 - DNA, 743 bp, synthetic construct
  misc_feature 1..743: note = Homo sapiens MANSC1 NM_018050 3'-UTR
  source 1..743: mol_type = other DNA
ggatggaactcggtgtctcttaattcatttagtaaccagaagcccaaatgcaatgagtttctgctgacttgctagtcttagcaggaggttgtattttgaagacaggaaaatgcccccttctgctttccttttttttttttggagacagagtcttgctttgttgcccaggctggagtgcagtagcacgatctcggctctcaccgcaacctccgtctcctgggttcaagcgattctcctgcctcagcctcctaagtatctgggattacaggcatgtgccaccacacctgggtgatttttgtatttttagtagagacggggtttcaccatgttggtcaggctggtctcaaactcctgacctagtgatccaccctcctcggcctcccaaagtgctgggattacaggcatgagccaccacagctggcccccttctgttttatgtttggtttttgagaaggaatgaagtgggaaccaaattaggtaattttgggtaatctgtctctaaaatattagctaaaaacaaagctctatgtaaagtaataaagtataattgccatataaatttcaaaattcaactggcttttatgcaaagaaacaggttaggacatctaggttccaattcattcacattcttggttccagataaaatcaactgtttatatcaatttctaatggatttgcttttctttttatatggattcctttaaaacttattccagatgtagttccttccaattaaatatttg

SEQ ID NO: 319 - DNA, 142 bp, synthetic construct
  misc_feature 1..142: note = Homo sapiens LTA4H 5'-UTR LTA4H-001 ENST00000228740
  source 1..142: mol_type = other DNA
aagaaacttcctttcccggcgtgcaccgcgaatccctcctcctcttctttacctctctccctcctcctcaggttctctatcgacgagtctggtagctgagcgttgggctgtaggtcgctgtgctgtgtgatcccccagagcc

SEQ ID NO: 320 - DNA, 88 bp, synthetic construct
  misc_feature 1..88: note = Homo sapiens DECR1 5'-UTR DECR1-001 ENST00000220764
  source 1..88: mol_type = other DNA
tccagccccgagaactttgttctttttgtcccgccccctgcgcccaaccgcctgcgccgccttccggcccgagttctggagactcaac

SEQ ID NO: 321 - DNA, 45 bp, synthetic construct
  misc_feature 1..45: note = Homo sapiens PIGK 5'-UTR
  source 1..45: mol_type = other DNA
actgcctccgccccttcaggtgcgggaagtctgaagccggtaaac

SEQ ID NO: 322 - DNA, 122 bp, synthetic construct
  misc_feature 1..122: note = Homo sapiens BRP44L 5'-UTR BRP44L-001
  source 1..122: mol_type = other DNA
gtcgtgaggcgggccttcgggctggctcgccgtcggctgccggggggttggccggggtgtcattggctctgggaagcggcagcagaggcagggaccactcggggtctggtgtcggcacagcc

SEQ ID NO: 323 - DNA, 117 bp, synthetic construct
  misc_feature 1..117: note = Homo sapiens ACADSB 5'-UTR ACADSB-004 NM_001609.3 ENST00000368869
  source 1..117: mol_type = other DNA
agggattaagggggggtgtgtgcggggcgggtactgagtgggcggggccttgctcgggtaactcccaggggctggctagagacccagaggcgcagagcggagaggcctgcggcgagg

SEQ ID NO: 324 - DNA, 166 bp, synthetic construct
  misc_feature 1..166: note = Homo sapiens SUPT3H 5'-UTR SUPT3H-006 ENST00000371459
  source 1..166: mol_type = other DNA
cacagccgagtcaccttttccctttctacactccacactctcagtcccccaccccgcccctttccaagcgtgtcccgggccgcagcagcagaaaccgcaccatctccacccccacattctcctcgcgggaagcgcagcagtgcctccaagggttcttaaagcagag

SEQ ID NO: 325 - DNA, 176 bp, synthetic construct
  misc_feature 1..176: note = Homo sapiens TMEM14A 5'-UTR NM_014051.3
  source 1..176: mol_type = other DNA
gtttccaggagggagcggcctttgctcagcgcgagacggctgggcgccgagtgggacagcgctggtgcggagactgcttccggactccaggtaccgcgcttggcggcagctggccccagacttctgtcttttcagctgcagtgaaggctcggggctgcagaattgcaaccttgcca

SEQ ID NO: 326 - DNA, 222 bp, synthetic construct
  misc_feature 1..222: note = Homo sapiens C9orf46 5'-UTR AF225420.1
  source 1..222: mol_type = other DNA
gagcgaggcccggtccctgcagcgggcgaaaggagcccgggcctggaggtttgcgtaccggtcgcctggtcccggcaccagcgccgcccagtgtggtttcccataaggaagctcttcttcctgcttggcttccacctttaacccttccacctgggagcgtcctctaacacattcagactacaagtccagacccaggagagcaaggcccagaaagaggtcaaa

SEQ ID NO: 327 - DNA, 227 bp, synthetic construct
  misc_feature 1..227: note = Homo sapiens ANXA4 5'-UTR NM_001153.3
  source 1..227: mol_type = other DNA
gccccaggtgcgcttcccctagagagggattttccggtctcgtgggcagaggaacaaccaggaacttgggctcagtctccaccccacagtggggcggatccgtcccggataagacccgctgtctggccctgagtagggtgtgacctccgcagccgcagaggaggagcgcagcccggcctcgaagaacttctgcttgggtggctgaactctgatcttgacctagagtc

SEQ ID NO: 328 - DNA, 123 bp, synthetic construct
  misc_feature 1..123: note = Homo sapiens IFI6 5'-UTR NM_022873.2
  source 1..123: mol_type = other DNA
ccagccttcagccggagaaccgtttactcgctgctgtgcccatctatcagcaggctccgggctgaagattgcttctcttctctcctccaaggtctagtgacggagcccgcgcgcggcgccacc

SEQ ID NO: 329 - DNA, 58 bp, synthetic construct
  misc_feature 1..58: note = Homo sapiens C2orf34 5'-UTR CAMKMT -008 ENST00000402247
  source 1..58: mol_type = other DNA
tcctggcaggggacgagctgcggcggtggcacctccgggtgtggaaggctccagtgag

SEQ ID NO: 330 - DNA, 104 bp, synthetic construct
  misc_feature 1..104: note = Homo sapiens C2orf34 5'-UTR NM_024766.3
  source 1..104: mol_type = other DNA
gagggtgccgggcgtcacaggtcctgacagggaagaagttggcaggtcctggcaggggacgagctgcggcggtggcacctccgggtgtggaaggctccagtgag

SEQ ID NO: 331 - DNA, 53 bp, synthetic construct
  misc_feature 1..53: note = Homo sapiens ALDH6A1 5'-UTR ALDH6A1-002 ENST00000350259
  source 1..53: mol_type = other DNA
agtgcttctgggcagtagaggcgcggggtgcggagctagggcggccgagagcc

SEQ ID NO: 332 - DNA, 117 bp, synthetic construct
  misc_feature 1..117: note = Homo sapiens CCDC53 5'-UTR CCDC53-002 ENST00000545679
  source 1..117: mol_type = other DNA
ggaagggccccggaggcgggcacttggggggaaagttgagacgtgattaccgggttgggcgggccccatctgggaggggtttgtgggtgaactcggggtccaccgcccgctgaggag

SEQ ID NO: 333 - DNA, 44 bp, synthetic construct
  misc_feature 1..44: note = Homo sapiens CASP1 5'-UTR NM_001257119.1
  source 1..44: mol_type = other DNA
atactttcagtttcagtcacacaagaagggaggagagaaaagcc

SEQ ID NO: 334 - DNA, 124 bp, synthetic construct
  misc_feature 1..124: note = Homo sapiens NDUFB6 5'-UTR NM_182739.2
  source 1..124: mol_type = other DNA
gtaataaccgcgcgcggcgctcggcgttcccgcaaggtcgctttgcagagcgggagcgcgcttaagtaactagtccgtagttcgagggtgcgccgtgtccttttgcgttggtaccagcggcgac

SEQ ID NO: 335 - DNA, 47 bp, synthetic construct
  misc_feature 1..47: note = Homo sapiens BCKDHB 5'-UTR BCKDHB-002 ENST00000369760
  source 1..47: mol_type = other DNA
aggcggcgtgcggctgcatagcctgagaatcccggtggtgagcgggg

SEQ ID NO: 336 - DNA, 39 bp, synthetic construct
  misc_feature 1..39: note = Homo sapiens BCKDHB 5'-UTR NM_001164783.1
  source 1..39: mol_type = other DNA
ctacgtgagtgccggaccgctgagtggttgttagccaag

SEQ ID NO: 337 - DNA, 234 bp, synthetic construct
  misc_feature 1..234: note = Homo sapiens BBS2 5'-UTR NM_031885.3
  source 1..234: mol_type = other DNA
cacagaaggcgccgaggctccaccgcgcagccgcaaaaagagcggacgggtctgcgccgccgcaggaggagcaggcggtacctggacgggttcgtcccgggctgtttcgcgtccggcctgaggcggctggggccgcgcaggtagtgtccctgcacttcttgcccgggcgcgtgaggccagctccgctgcgcttgtctccagcttccagccctcctcccctaagccgccgccatc

SEQ ID NO: 338 - DNA, 153 bp, synthetic construct
  misc_feature 1..153: note = Homo sapiens HERC5 5'-UTR HERC5-001 ENST00000264350
  source 1..153: mol_type = other DNA
tcagtagctgaggctgcggttccccgacgccacgcagctgcgcgcagctggttcccgctctgcagcgcaacgcctgaggcagtgggcgcgctcagtcccgggaccaggcgttctctcctctcgcctctgggcctgggaccccgcaaagcggcg

SEQ ID NO: 339 - DNA, 65 bp, synthetic construct
  misc_feature 1..65: note = Homo sapiens FAM175A 5'-UTR NM_139076.2
  source 1..65: mol_type = other DNA
accacagggtcttgcctccgcgcgccccgccctcgtcctcttgtgtagcctgaggcggcggtagc

SEQ ID NO: 340 - DNA, 82 bp, synthetic construct
  misc_feature 1..82: note = Homo sapiens NT5DC1 5'-UTR NT5DC1-002 ENST00000319550
  source 1..82: mol_type = other DNA
cggtcctgtcccgcagcgtcccgccagccagctccttgcacccttcgcggccgaggcgctccctggtgctccccgcgcagcc

SEQ ID NO: 341 - DNA, 246 bp, synthetic construct
  misc_feature 1..246: note = Homo sapiens RAB7A 5'-UTR RAB7A-001 ENST00000265062
  source 1..246: mol_type = other DNA
gtctcgtgacaggtacttccgctcggggcggcggcggtggcggaagtgggagcgggcctggagtcttggccataaagcctgaggcggcggcagcggcggagttggcggcttggagagctcgggagagttccctggaaccagaacttggaccttctcgcttctgtcctccgtttagtctcctcctcggcgggagccctcgcgacgcgcccggcccggagcccccagcgcagcggccgcgtttgaagg

SEQ ID NO: 342 - DNA, 128 bp, synthetic construct
  misc_feature 1..128: note = Homo sapiens AGA 5'-UTR AGA-001 ENST00000264595
  source 1..128: mol_type = other DNA
agggacgcctgagcgaacccccgagagagcgggcgtgggcgccaggcgggcggggcactggggattaattgttcggcgatcgctggctgccgggacttttctcgcgctggtctcttcggtggtcaggg

SEQ ID NO: 343 - DNA, 103 bp, synthetic construct
  misc_feature 1..103: note = Homo sapiens TPK1 5'-UTR TPK1-001 ENST00000360057
  source 1..103: mol_type = other DNA
aaggctcctcagccgagcgccgagcggtcgatcgccgtagctcccgcagcctgcgatctccagtctgtggctcctaccagccattgtaggccaataatccgtt

SEQ ID NO: 344 - DNA, 79 bp, synthetic construct
  misc_feature 1..79: note = Homo sapiens MBNL3 5'-UTR MBNL3-001 ENST00000370839
  source 1..79: mol_type = other DNA
aattcatttttaatcctttaatagtccacagtaatattgtcctaaagagggtacattggattttaattttgctttcaat

SEQ ID NO: 345 - DNA, 129 bp, synthetic construct
  misc_feature 1..129: note = Homo sapiens MCCC2 5'-UTR MCCC2-001 ENST00000340941
  source 1..129: mol_type = other DNA
agaatcagagaaaccttctctggggctgcaaggacctgagctcagcttccgccccagccagggaagcggcaggggaaagcaccggctccaggccagcgtgggccgctctctcgctcggtgcccgccgcc

SEQ ID NO: 346 - DNA, 89 bp, synthetic construct
  misc_feature 1..89: note = Homo sapiens CAT 5'-UTR CAT-001 ENST00000241052
  source 1..89: mol_type = other DNA
actcggggcaacaggcagatttgcctgctgagggtggagacccacgagccgaggcctcctgcagtgttctgcacagcaaaccgcacgct

SEQ ID NO: 347 - DNA, 142 bp, synthetic construct
  misc_feature 1..142: note = Homo sapiens ANAPC4 5'-UTR ANAPC4-001 ENST00000315368
  source 1..142: mol_type = other DNA
cccgacgccggaagtgcctggagcgcgcgacagcggcggggcggggcggcctggaggctgtggcgcgcggccggcagagggaggggagaggccactggggccgtgttagtctgccggtggggactcttgcagggccgtcccc

SEQ ID NO: 348 - DNA, 24 bp, synthetic construct
  misc_feature 1..24: note = Homo sapiens PHKB 5'-UTR PHKB-002 ENST00000323584
  source 1..24: mol_type = other DNA
ggccaaggcggcgaccggagcgcg

SEQ ID NO: 349 - DNA, 25 bp, synthetic construct
  misc_feature 1..25: note = Homo sapiens ABCB7 5'-UTR ABCB7-001 ENST00000253577
  source 1..25: mol_type = other DNA
ctcggttcctctttcctcgctcaag

SEQ ID NO: 350 - DNA, 133 bp, synthetic construct
  misc_feature 1..133: note = Homo sapiens GPD2 5'-UTR GPD2-002 ENST00000438166
  source 1..133: mol_type = other DNA
cccgcgcgcctcgctgggagcacccgggccgaggctctgattctggggggaggccgactccaccctggctggaggaactgggtgctcctgcccgctggcccctcgcgcgtgaggatctatctcaggctaagaa

SEQ ID NO: 351 - DNA, 117 bp, synthetic construct
  misc_feature 1..117: note = Homo sapiens TMEM38B 5'-UTR TMEM38B-001 ENST00000374692
  source 1..117: mol_type = other DNA
gctggagccggcgcggaggagcgggcggccgcggctgtgccctctcctactcctcaccgcgcgagcgcggggaaccagtagccgcggctgcttcggttgccgcggtcggtggtcgtt

SEQ ID NO: 352 - DNA, 206 bp, synthetic construct
  misc_feature 1..206: note = Homo sapiens NFU1 5'-UTR NM_001002755.2
  source 1..206: mol_type = other DNA
gggaaaggttccccggcctctcttggtcagggtgacgcagtagcctgcaaacctcggcgcgtaggccaccgcacttatccgcagcaggaccgcccgcagccggtagggtgggctcttcccagtgcccgcccagctaccggccagcctgcggctgcgcagatctttcgtggttctgtcagggagacccttaggcactccggactaag

SEQ ID NO: 353 - DNA, 99 bp, synthetic construct
  misc_feature 1..99: note = Homo sapiens LOC128322/NUTF2 5'-UTR NM_005796.1
  source 1..99: mol_type = other DNA
ggaagggacagtcggccgcagaccgcgctgggttgccgctgccgctgccgccatcgtgccagcccctcgggtctccgtgaggccgggtgacgctccaga

SEQ ID NO: 354 - DNA, 55 bp, synthetic construct
  misc_feature 1..55: note = Homo sapiens NUBPL 5'-UTR NM_025152.2
  source 1..55: mol_type = other DNA
actccgcgccacccgcgacagtttcccagcagggctcacagcagcgttccgcgtc

SEQ ID NO: 355 - DNA, 75 bp, synthetic construct
  misc_feature 1..75: note = Homo sapiens LANCL1 5'-UTR LANCL1-004 ENST00000233714
  source 1..75: mol_type = other DNA
gagaagggcttcaggacgcgggaggcgcacttgcttcaagtcgcgggcgtgggaacggggcttgcttccggcgtc

SEQ ID NO: 356 - DNA, 204 bp, synthetic construct
  misc_feature 1..204: note = Homo sapiens PIR 5'-UTR PIR-002 ENST00000380420
  source 1..204: mol_type = other DNA
cctcccgcctcctctaggccgccggccgcgaagcgctgagtcacggtgaggctactggacccacactctcttaacctgccctccctgcactcgctcccggcggctcttcgcgtcacccccgccgctaaggctccaggtgccgctaccgcagcccctccatcctctacagctcagcatcagaacactctctttttagactccgat

SEQ ID NO: 357 - DNA, 65 bp, synthetic construct
  misc_feature 1..65: note = Homo sapiens CTBS 5'-UTR NM_004388.2
  source 1..65: mol_type = other DNA
gacgcgcagcaggccccgcccacccaggcggtaggaacccactccggcccgctagacctgctgct

SEQ ID NO: 358 - DNA, 314 bp, synthetic construct
  misc_feature 1..314: note = Homo sapiens GSTM4 5'-UTR NM_000850.4
  source 1..314: mol_type = other DNA
aagctggcgaggccgagcccctcctagtgcttccggaccttgctccctgaacactcggaggtggcggtggatcttactccttccagccagtgaggatccagcaacctgctccgtgcctcccgcgcctgttggttggaagtgacgaccttgaagatcggccggttggaagtgacgaccttgaagatcggcgggcgcagcggggccgagggggcgggtctggcgctaggtccagcccctgcgtgccgggaaccccagaggaggtcgcagttcagcccagctgaggcctgtctgcagaatcgacaccaaccagcatc

SEQ ID NO: 359 - DNA, 73 bp, synthetic construct
  misc_feature 1..73: note = Mus musculus Ndufa1 5'-UTR Ndufa1-001 ENSMUST00000016571
  source 1..73: mol_type = other DNA
gccggaagagaggtaaagccgggtcacctctgaggagccggtgacgggttggcgtgcgagtaacggtgcggag

SEQ ID NO: 360 - DNA, 105 bp, synthetic construct
  misc_feature 1..105: note = Mus musculus Atp5e 5'-UTR NM_025983
  source 1..105: mol_type = other DNA
cccaccccttccgctactcaggcctgaccttcctgctgccgggccggtttgaggctactctgaagcgacccagcggttctgcccgacgcgcccgctcgagacacc

SEQ ID NO: 361 - DNA, 100 bp, synthetic construct
  misc_feature 1..100: note = Mus musculus Gstm5 5'-UTR NM_010360
  source 1..100: mol_type = other DNA
gagacagttcggtcgcgtcagcccggcccacagcgtccagtataaagttagccgcccacagtccatcgctgtatccccgaaggggctaagatcgcccaaa

SEQ ID NO: 362 - DNA, 49 bp, synthetic construct
  misc_feature 1..49: note = Mus musculus Cbr2 5'-UTR NM_007621
  source 1..49: mol_type = other DNA
ataaaagctgagcccatctcttgcttcggaagaagctggtgtcagcagc

SEQ ID NO: 363 - DNA, 78 bp, synthetic construct
  misc_feature 1..78: note = Mus musculus Anapc13 5'-UTR NM_181394
  source 1..78: mol_type = other DNA
gtgacccagaagaagggcggggccgggaggaagccgacgcgcgcgcagtgggcctgacaagatcaaagctgcaggagg

SEQ ID NO: 364 - DNA, 16 bp, synthetic construct
  misc_feature 1..16: note = Mus musculus Ndufa7 5'-UTR NM_023202
  source 1..16: mol_type = other DNA
tcggagcggaaggaat

SEQ ID NO: 365 - DNA, 17 bp, synthetic construct
  misc_feature 1..17: note = Mus musculus Atp5k 5'-UTR NM_007507
  source 1..17: mol_type = other DNA
cgaaggtcacggacaaa

SEQ ID NO: 366 - DNA, 67 bp, synthetic construct
  misc_feature 1..67: note = Mus musculus Cox4i1 5'-UTR NM_009941
  source 1..67: mol_type = other DNA
cttccggtcgcgagcaccccagggtgtagagggcggtcgcggcggtcgcctgggcagcggtggcaga

SEQ ID NO: 367 - DNA, 36 bp, synthetic construct
  misc_feature 1..36: note = Mus musculus Ndufs6 5'-UTR NM_010888
  source 1..36: mol_type = other DNA
ttggtacgacgcgtggggtcaagggtcaccggcaag

SEQ ID NO: 368 - DNA, 90 bp, synthetic construct
  misc_feature 1..90: note = Mus musculus Sec61b 5'-UTR NM_024171
  source 1..90: mol_type = other DNA
agagcctgtatctacgagagttctgagtgctcggcaacttcacgacttccctcttcctgcctcctgtgcccaccgttcttaggcatcagc

SEQ ID NO: 369 - DNA, 55 bp, synthetic construct
  misc_feature 1..55: note = Mus musculus Snrpd2 5'-UTR NM_026943
  source 1..55: mol_type = other DNA
aaggctggagcaacgcgcttggaggcgggagtgatctgcgagcgaaacctacacc

SEQ ID NO: 370 - DNA, 54 bp, synthetic construct
  misc_feature 1..54: note = Mus musculus Mgst3 5'-UTR NM_025569
  source 1..54: mol_type = other DNA
actgctgtgcttctcaggtctgtaccaggcgcacgaaggtgagccagagccaag

SEQ ID NO: 371 - DNA, 54 bp, synthetic construct; identical to SEQ ID NO: 25
  misc_feature 1..54: note = Mus musculus Mp68 (2010107E04Rik) 5'-UTR NM_027360
  source 1..54: mol_type = other DNA

SEQ ID NO: 372 - DNA, 92 bp, synthetic construct
  misc_feature 1..92: note = Mus musculus Prdx4-001, 5'-UTR NM_016764
  source 1..92: mol_type = other DNA
gcgcggtctccagcgcgccgttttagctggctgcctggcggcaggggactctgtgctttagcagagggacgtgttttcgcgcttgcttggtc

SEQ ID NO: 373 - DNA, 215 bp, synthetic construct
  misc_feature 1..215: note = Mus musculus Pgcp 5'-UTR NM_176073
  source 1..215: mol_type = other DNA
gctgtcctggcacacaaagaagccaggcctgcagactactggggctccgggctgttcctgaggcctctggaggcccgccctgtggctccagtgcgctctgaggaccttcctggtcccgcccccgaacgtgcctgtggtctgcaggcctcaccgggtgttgtggccgctgctgctccgcagagcctcgtgatcaggaagaaaagcaactaggaaca

SEQ ID NO: 374 - DNA, 43 bp, synthetic construct
  misc_feature 1..43: note = Mus musculus Myeov2 5'-UTR NM_001163425
  source 1..43: mol_type = other DNA
agaaggggctggccggaagtgagcgcaacgccgccttgtcgag

SEQ ID NO: 375 - DNA, 81 bp, synthetic construct; identical to SEQ ID NO: 28
  misc_feature 1..81: note = Mus musculus Ndufa4 5'-UTR NM_010886
  source 1..81: mol_type = other DNA

SEQ ID NO: 376 - DNA, 76 bp, synthetic construct
  misc_feature 1..76: note = Mus musculus Ndufs5 5'-UTR NM_001030274
  source 1..76: mol_type = other DNA
acggcaggcgtctgcgtcctcccgcagccggcggtcgggaattgcaccagggacctgacaagggcactgcagagcc

SEQ ID NO: 377 - DNA, 198 bp, synthetic construct
  misc_feature 1..198: note = Mus musculus Gstm1 5'-UTR NM_010358
  source 1..198: mol_type = other DNA
ctgccttccgctttagggtctgctgctctggttacagacctaggaaggggagtgcctaattgggattggtgcagggttgggagggacccgctgttttgtcctgcccacgtttctctagtagtctgtataaagtcacaactccaaacacacaggtcagtcctgctgaagccagtttgagaagaccacagcaccagcacc

SEQ ID NO: 378 - DNA, 75 bp, synthetic construct
  misc_feature 1..75: note = Mus musculus Atp5o 5'-UTR NM_138597
  source 1..75: mol_type = other DNA
ctggcgcgcgcgcgtgcgctctggcgccagtagtctcttttcatttgggtttgacctacagccgcccgggaaaag

SEQ ID NO: 379 - DNA, 101 bp, synthetic construct
  misc_feature 1..101: note = Mus musculus Tspo 5'-UTR NM_009775
  source 1..101: mol_type = other DNA
gtcagcggctaccaacctctgtgcgcagtgtccttcacggaacaaccagcgactgcgtgagcggggctgtggatctttccagaacatcagttgcaatcacc

SEQ ID NO: 380 - DNA, 62 bp, synthetic construct
  misc_feature 1..62: note = Mus musculus Taldo1 5'-UTR NM_011528
  source 1..62: mol_type = other DNA
gacgcgcggggcattgtgggttagcacgcaccggctaccgcctcagctgttcgcgtttcgcc

SEQ ID NO: 381 - DNA, 89 bp, synthetic construct
  misc_feature 1..89: note = Mus musculus Bloc1s1 5'-UTR NM_015740
  source 1..89: mol_type = other DNA
gtgacgccttccgggtgagccaaggcatagtccagttcctgcagccttagggaggggtccgccgtgcccacacccagccagactcgacc

SEQ ID NO: 382 - DNA, 57 bp, synthetic construct
  misc_feature 1..57: note = Mus musculus Hexa 5'-UTR NM_010421
  source 1..57: mol_type = other DNA
agctgaccggggctcacgtgggctcagcctgctggaaggggagctggccggtgggcc

SEQ ID NO: 383 - RNA, 1810 nt, synthetic construct
  misc_feature 1..1810: note = 32L4 - PpLuc(GC) - A64-C30-hSL
  source 1..1810: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 384 - RNA, 1927 nt, synthetic construct
  misc_feature 1..1927: note = PpLuc(GC) - morn2- A64 - C30 - hSL
  source 1..1927: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtacctgctgccttaacgctgagatgtggcctctgcaaccccccttaggcaaagcaactgaaccttctgctaaagtgacctgccctcttccgtaagtccaataaagttgtcatgcacccagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 385 - RNA, 1943 nt, synthetic construct
  misc_feature 1..1943: note = PpLuc(GC) - ndufa1- A64 - C30 - hSL
  source 1..1943: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtggaagcattttcctggctgattaaaagaaattactcagctatggtcatctgttcctgttagaaggctatgcagcatattatatactatgcgcatgttatgaaatgcataataaaaaattttaaaaaatctaaaagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 386 - RNA, 1941 nt, synthetic construct
  misc_feature 1..1941: note = PpLuc(GC) - NDUFA1- A64 - C30 - hSL
  source 1..1941: mol_type = other RNA
gggagaaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtggaagcattttcctgattgatgaaaaaaataactcagttatggccatctacccctgctagaaggttacagtgtattatgtagcatgcaatgtgttatgtagtgcttaataaaaataaaatgaaaaaaatgcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 387 - RNA, 1861 nt, synthetic construct
  misc_feature 1..1861: note = Mp68 - PpLuc(GC) - A64 - C30 - hSL
  source 1..1861: mol_type = other RNA
gggctttcccattctgtagcagaatttggtgttgcctgtggtcttggtcccgcggagaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 388 - RNA, 1888 nt, synthetic construct
  misc_feature 1..1888: note = Ndufa4 - PpLuc(GC) - A64 - C30 - hSL
  source 1..1888: mol_type = other RNA
ggggtccgctcagccaggttgcagaagcggcttagcgtgtgtcctaatcttctctctgcgtgtaggtaggcctgtgccgcaaacaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

(19) United States
(12) Patent Application Publication
(10) Pub. No.: US 2024/0294928 A1
(43) Pub. Date: Sep. 5, 2024

(54) ARTIFICIAL NUCLEIC ACID MOLECULES

(71) Applicant: CureVac SE, Tübingen (DE)

(72) Inventors: Stefanie GRUND, Stuttgart (DE); Thomas SCHLAKE, Gundelfingen (DE)

(73) Assignee: CureVac SE, Tübingen (DE)

(21) Appl. No.: 18/415,477

(22) Filed: Jan. 17, 2024

Related U.S. Application Data
(62) Division of application No. 16/523,214, filed on Jul. 26, 2019, now abandoned.
Application No. 16/523,214 is a division of application No. 15/540,610, filed on Jun. 29, 2017, now abandoned.

(30) Foreign Application Priority Data
Dec. 30, 2014 (WO) PCT/EP2014/003479

(51) Int. Cl. C12N 15/68; A61K 48/00; C12N 15/67; C12N 15/85

(52) U.S. Cl. CPC C12N 15/68; A61K 48/0066; C12N 15/67; C12N 15/85; C12N 2830/50; C12N 2840/105

(57) ABSTRACT

The invention relates to an artificial nucleic acid molecule comprising at least one open reading frame and at least one 3′-untranslated region element (3′-UTR element) and/or at least one 5′-untranslated region element (5′-UTR element), wherein the at least one 3′-UTR element and/or the at least one 5′-UTR element prolongs and/or increases protein production from said artificial nucleic acid molecule and wherein the at least one 3′-UTR element and/or the at least one 5′-UTR element is derived from a stable mRNA. The invention further relates to the use of such an artificial nucleic acid molecule in gene therapy and/or genetic vaccination. Furthermore, methods for identifying a 3′-UTR element and/or a 5′-UTR derived from a stable mRNA element are disclosed.

This application is a divisional of U.S. application Ser. No. 16/523,214, filed Jul. 26, 2019, which is a divisional of U.S. application Ser. No. 15/540,610, filed Jun. 29, 2017, now abandoned, which is a national phase application under 35 U.S.C. § 371 of International Application No. PCT/EP2015/081366, filed Dec. 29, 2015, which claims the priority of International Application No. PCT/EP2014/003479, filed on Dec. 30, 2014, each of which is incorporated herein by reference.

This invention was made with government support under Agreement No. HR0011-11-3-0001 awarded by DARPA. The Government has certain rights in the invention.

This application contains a Sequence Listing XML, which has been submitted electronically and is hereby incorporated by reference in its entirety. Said Sequence Listing XML, created on Jan. 17, 2024, is named CRVCP0167USD2.xml and is 640,058 bytes in size.

The invention relates to artificial nucleic acid molecules comprising an open reading frame, a 3′-untranslated region element (3′-UTR element) and/or a 5′-untranslated region element (5′-UTR element) and optionally a poly(A) sequence and/or a polyadenylation-signal. The invention relates further to a vector comprising a 3′-UTR element and/or a 5′-UTR element, to a cell comprising the artificial nucleic acid molecule or the vector, to a pharmaceutical composition comprising the artificial nucleic acid molecule or the vector and to a kit comprising the artificial nucleic acid molecule, the vector and/or the pharmaceutical composition, preferably for use in the field of gene therapy and/or genetic vaccination.

Gene therapy and genetic vaccination belong to the most promising and quickly developing methods of modern medicine. They may provide highly specific and individual options for therapy of a large variety of diseases. Particularly, inherited genetic diseases but also autoimmune diseases, cancerous or tumour-related diseases as well as inflammatory diseases may be the subject of such treatment approaches. Also, it is envisaged to prevent early onset of such diseases by these approaches.

The main conceptual rational behind gene therapy is appropriate modulation of impaired gene expression associated with pathological conditions of specific diseases. Pathologically altered gene expression may result in lack or overproduction of essential gene products, for example, signalling factors such as hormones, housekeeping factors, metabolic enzymes, structural proteins or the like. Altered gene expression may not only be due to mis-regulation of transcription and/or translation, but also due to mutations within the ORF coding for a particular protein. Pathological mutations may be caused by e.g. chromosomal aberration, or by more specific mutations, such as point or frame-shift-mutations, all of them resulting in limited functionality and, potentially, total loss of function of the gene product. However, misregulation of transcription or translation may also occur, if mutations affect genes encoding proteins which are involved in the transcriptional or translational machinery of the cell. Such mutations may lead to pathological up- or down-regulation of genes which are—as such—functional. Genes encoding gene products which exert such regulating functions, may be, e.g., transcription factors, signal receptors, messenger proteins or the like. However, loss of function of such genes encoding regulatory proteins may, under certain circumstances, be reversed by artificial introduction of other factors acting further downstream of the impaired gene product. Such gene defects may also be compensated by gene therapy via substitution of the affected gene itself.

Genetic vaccination allows evoking a desired immune response to selected antigens, such as characteristic components of bacterial surfaces, viral particles, tumour antigens or the like. Generally, vaccination is one of the pivotal achievements of modern medicine. However, effective vaccines are currently available only for a limited number of diseases. Accordingly, infections that are not preventable by vaccination still affect millions of people every year.

Commonly, vaccines may be subdivided into “first”, “second” and “third” generation vaccines. “First generation” vaccines are, typically, whole-organism vaccines. They are based on either live and attenuated or killed pathogens, e.g. viruses, bacteria or the like. The major drawback of live and attenuated vaccines is the risk for a reversion to life-threatening variants. Thus, although attenuated, such pathogens may still intrinsically bear unpredictable risks. Killed pathogens may not be as effective as desired for generating a specific immune response. In order to minimize these risks, “second generation” vaccines were developed. These are, typically, subunit vaccines, consisting of defined antigens or recombinant protein components which are derived from pathogens.

Genetic vaccines, i.e. vaccines for genetic vaccination, are usually understood as “third generation” vaccines. They are typically composed of genetically engineered nucleic acid molecules which allow expression of peptide or protein (antigen) fragments characteristic for a pathogen or a tumor antigen in vivo. Genetic vaccines are expressed upon administration to a patient after uptake by target cells. Expression of the administered nucleic acids results in production of the encoded proteins. In the event these proteins are recognized as foreign by the patient's immune system, an immune response is triggered.

As can be seen from the above, both methods, gene therapy and genetic vaccination, are essentially based on the administration of nucleic acid molecules to a patient and subsequent transcription and/or translation of the encoded genetic information. Alternatively, genetic vaccination or gene therapy may also comprise methods which include isolation of specific body cells from a patient to be treated, subsequent in ex vivo transfection of such cells, and re-administration of the treated cells to the patient.

DNA as well as RNA may be used as nucleic acid molecules for administration in the context of gene therapy or genetic vaccination. DNA is known to be relatively stable and easy to handle. However, the use of DNA bears the risk of undesired insertion of the administered DNA-fragments into the patient's genome potentially resulting mutagenic events such as in loss of function of the impaired genes. As a further risk, the undesired generation of anti-DNA antibodies has emerged. Another drawback is the limited expression level of the encoded peptide or protein that is achievable upon DNA administration because the DNA must enter the nucleus in order to be transcribed before the resulting mRNA can be translated. Among other reasons, the expression level of the administered DNA will be dependent on the presence of specific transcription factors which regulate DNA transcription. In the absence of such factors, DNA transcription will not yield satisfying amounts of RNA. As a result, the level of translated peptide or protein obtained is limited.

By using RNA instead of DNA for gene therapy or genetic vaccination, the risk of undesired genomic integration and generation of anti-DNA antibodies is minimized or avoided. However, RNA is considered to be a rather unstable molecular species which may readily be degraded by ubiquitous RNAses.

Typically, RNA degradation contributes to the regulation of the RNA half-life time. That effect was considered and proven to fine tune the regulation of eukaryotic gene expression (Friedel et al., 2009. Conserved principles of mammalian transcriptional regulation revealed by RNA half-life, Nucleic Acid Research 37(17): 1-12). Accordingly, each naturally occurring mRNA has its individual half-life depending on the gene from which the mRNA is derived and in which cell type it is expressed. It contributes to the regulation of the expression level of this gene. Unstable RNAs are important to realize transient gene expression at distinct points in time. However, long-lived RNAs may be associated with accumulation of distinct proteins or continuous expression of genes. In vivo, the half-life of mRNAs may also be dependent on environmental factors, such as hormonal treatment, as has been shown, e.g., for insulin-like growth factor I, actin, and albumin mRNA (Johnson et al., Newly synthesized RNA: Simultaneous measurement in intact cells of transcription rates and RNA stability of insulin-like growth factor I, actin, and albumin in growth hormone-stimulated hepatocytes, Proc. Natl. Acad. Sci., Vol. 88, pp. 5287-5291, 1991).

For gene therapy and genetic vaccination, usually stable RNA is desired. This is, on the one hand, due to the fact that it is usually desired that the product encoded by the RNA sequence accumulates in vivo. On the other hand, the RNA has to maintain its structural and functional integrity when prepared for a suitable dosage form, in the course of its storage, and when administered. Thus, efforts were made to provide stable RNA molecules for gene therapy or genetic vaccination in order to prevent them from being subject to early degradation or decay.

It has been reported that the G/C-content of nucleic acid molecules may influence their stability. Thus, nucleic acids comprising an increased amount of guanine (G) and/or cytosine (C) residues may be functionally more stable than nucleic acids containing a large amount of adenine (A) and thymine (T) or uracil (U) nucleotides. In this context, WO02/098443 provides a pharmaceutical composition containing an mRNA that is stabilised by sequence modifications in the coding region. Such a sequence modification takes advantage of the degeneracy of the genetic code. Accordingly, codons which contain a less favourable combination of nucleotides (less favourable in terms of RNA stability) may be substituted by alternative codons without altering the encoded amino acid sequence. This method of RNA stabilization is limited by the provisions of the specific nucleotide sequence of each single RNA molecule which is not allowed to leave the space of the desired amino acid sequence. Also, that approach is restricted to coding regions of the RNA.

As an alternative option for mRNA stabilisation, it has been found that naturally occurring eukaryotic mRNA molecules contain characteristic stabilising elements. For example, they may comprise so-called untranslated regions (UTR) at their 5′-end (5′-UTR) and/or at their 3′-end (3′-UTR) as well as other structural features, such as a 5′-cap structure or a 3′-poly(A) tail. Both, 5′-UTR and 3′-UTR are typically transcribed from the genomic DNA and are, thus, an element of the premature mRNA. Characteristic structural features of mature mRNA, such as the 5′-cap and the 3′-poly(A) tail (also called poly(A) tail or poly(A) sequence) are usually added to the transcribed (premature) mRNA during mRNA processing.

A 3′-poly(A) tail is typically a monotonous sequence stretch of adenosine nucleotides added to the 3-end of the transcribed mRNA. It may comprise up to about 400 adenosine nucleotides. It was found that the length of such a 3′-poly(A) tail is a potentially critical element for the stability of the individual mRNA.

Also, it was shown that the 3′-UTR of α-globin mRNA may be an important factor for the well-known stability of α-globin mRNA (Rodgers et al., Regulated α-globin mRNA decay is a cytoplasmic event proceeding through 3′-to-5′ exosome-dependent decapping, RNA, 8, pp. 1526-1537, 2002). The 3′-UTR of α-globin mRNA is apparently involved in the formation of a specific ribonucleoprotein-complex, the α-complex, whose presence correlates with mRNA stability in vitro (Wang et al., An mRNA stability complex functions with poly(A)-binding protein to stabilize mRNA in vitro, Molecular and Cellular biology, Vol 19, No. 7, July 1999, p. 4552-4560).

An interesting regulatory function has further been demonstrated for the UTRs in ribosomal protein mRNAs: while the 5′-UTR of ribosomal protein mRNAs controls the growth-associated translation of the mRNA, the stringency of that regulation is conferred by the respective 3′-UTR in ribosomal protein mRNAs (Ledda et al., Effect of the 3′-UTR length on the translational regulation of 5′-terminal oligopyrimidine mRNAs, Gene, Vol. 344, 2005, p. 213-220). This mechanism contributes to the specific expression pattern of ribosomal proteins, which are typically transcribed in a constant manner so that some ribosomal protein mRNAs such as ribosomal protein S9 or ribosomal protein L32 are referred to as housekeeping genes (Janovick-Guretzky et al., Housekeeping Gene Expression in Bovine Liver is Affected by Physiological State, Feed Intake, and Dietary Treatment, J. Dairy Sci., Vol. 90, 2007, p. 2246-2252). The growth-associated expression pattern of ribosomal proteins is thus mainly due to regulation on the level of translation.

Irrespective of factors influencing mRNA stability, effective translation of the administered nucleic acid molecules by the target cells or tissue is crucial for any approach using nucleic acid molecules for gene therapy or genetic vaccination. As can be seen from the examples cited above, along with the regulation of stability, also translation of the majority of mRNAs is regulated by structural features like UTRs, 5′-cap and 3′-poly(A) tail. In this context, it has been reported that the length of the poly(A) tail may play an important role for translational efficiency as well. Stabilizing 3′-elements, however, may also have an attenuating effect on translation.

It is the object of the invention to provide nucleic acid molecules which may be suitable for application in gene therapy and/or genetic vaccination. Particularly, it is the object of the invention to provide an mRNA species which is stabilized against preterm degradation or decay without exhibiting significant functional loss in translational efficiency. It is also an object of the invention to provide an artificial nucleic acid molecule, preferably an mRNA, which is characterized by enhanced expression of the respective protein encoded by said nucleic acid molecule. One particular object of the invention is the provision of an mRNA, wherein the efficiency of translation of the respective encoded protein is enhanced. Another object of the present invention is to provide nucleic acid molecules coding for such a superior mRNA species which may be amenable for use in gene therapy and/or genetic vaccination. It is a further object of the present invention to provide a pharmaceutical composition for use in gene therapy and/or genetic vaccination. In summary, it is the object of the present invention to provide improved nucleic acid species which overcome the above discussed disadvantages of the prior art by a cost-effective and straight-forward approach.

The object underlying the present invention is solved by the claimed subject matter.

For the sake of clarity and readability the following definitions are provided. Any technical feature mentioned for these definitions may be read on each and every embodiment of the invention. Additional definitions and explanations may be specifically provided in the context of these embodiments.

Adaptive immune response: The adaptive immune response is typically understood to be an antigen-specific response of the immune system. Antigen specificity allows for the generation of responses that are tailored to specific pathogens or pathogen-infected cells. The ability to mount these tailored responses is usually maintained in the body by “memory cells”. Should a pathogen infect the body more than once, these specific memory cells are used to quickly eliminate it. In this context, the first step of an adaptive immune response is the activation of naive antigen-specific T cells or different immune cells able to induce an antigen-specific immune response by antigen-presenting cells. This occurs in the lymphoid tissues and organs through which naïve T cells are constantly passing. The three cell types that may serve as antigen-presenting cells are dendritic cells, macrophages, and B cells. Each of these cells has a distinct function in eliciting immune responses. Dendritic cells may take up antigens by phagocytosis and macropinocytosis and may become stimulated by contact with e.g. a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. MHC-molecules are, typically, responsible for presentation of an antigen to T-cells. Therein, presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind the antigen directly, but instead recognize short peptide fragments e.g. of pathogen-derived protein antigens, e.g. so-called epitopes, which are bound to MHC molecules on the surfaces of other cells.

Adaptive immune system: The adaptive immune system is essentially dedicated to eliminate or prevent pathogenic growth. It typically regulates the adaptive immune response by providing the vertebrate immune system with the ability to recognize and remember specific pathogens (to generate immunity), and to mount stronger attacks each time the pathogen is encountered. The system is highly adaptable because of somatic hypermutation (a process of accelerated somatic mutations), and V(D)J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of such a cell will then inherit genes encoding the same receptor specificity, including the Memory B cells and Memory T cells that are the keys to long-lived specific immunity.

Adjuvant/adjuvant component: An adjuvant or an adjuvant component in the broadest sense is typically a pharmacological and/or immunological agent that may modify, e.g. enhance, the effect of other agents, such as a drug or vaccine. It is to be interpreted in a broad sense and refers to a broad spectrum of substances. Typically, these substances are able to increase the immunogenicity of antigens. For example, adjuvants may be recognized by the innate immune systems and, e.g., may elicit an innate immune response. “Adjuvants” typically do not elicit an adaptive immune response. Insofar, “adjuvants” do not qualify as antigens. Their mode of action is distinct from the effects triggered by antigens resulting in an adaptive immune response.

Antigen: In the context of the present invention “antigen” refers typically to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. In the sense of the present invention an antigen may be the product of translation of a provided nucleic acid molecule, preferably an mRNA as defined herein. In this context, also fragments, variants and derivatives of peptides and proteins comprising at least one epitope are understood as antigens. In the context of the present invention, tumour antigens and pathogenic antigens as defined herein are particularly preferred.

Artificial nucleic acid molecule: An artificial nucleic acid molecule may typically be understood to be a nucleic acid molecule, e.g. a DNA or an RNA, that does not occur naturally. In other words, an artificial nucleic acid molecule may be understood as a non-natural nucleic acid molecule. Such nucleic acid molecule may be non-natural due to its individual sequence (which does not occur naturally) and/or due to other modifications, e.g. structural modifications of nucleotides which do not occur naturally. An artificial nucleic acid molecule may be a DNA molecule, an RNA molecule or a hybrid-molecule comprising DNA and RNA portions. Typically, artificial nucleic acid molecules may be designed and/or generated by genetic engineering methods to correspond to a desired artificial sequence of nucleotides (heterologous sequence). In this context an artificial sequence is usually a sequence that may not occur naturally, i.e. it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

Bicistronic RNA, multicistronic RNA: A bicistronic or multicistronic RNA is typically an RNA, preferably an mRNA, that typically may have two (bicistronic) or more (multicistronic) open reading frames (ORF). An open reading frame in this context is a sequence of codons that is translatable into a peptide or protein.

Carrier/polymeric carrier: A carrier in the context of the invention may typically be a compound that facilitates transport and/or complexation of another compound (cargo). A polymeric carrier is typically a carrier that is formed of a polymer. A carrier may be associated to its cargo by covalent or non-covalent interaction. A carrier may transport nucleic acids, e.g. RNA or DNA, to the target cells. The carrier may—for some embodiments—be a cationic component.

Cationic component: The term “cationic component” typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic component may be any positively charged compound or polymer, preferably a cationic peptide or protein which is positively charged under physiological conditions, particularly under physiological conditions in vivo. A “cationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the conditions given.

5′-cap: A 5-cap is an entity, typically a modified nucleotide entity, which generally “caps” the 5′-end of a mature mRNA. A 5-cap may typically be formed by a modified nucleotide, particularly by a derivative of a guanine nucleotide. Preferably, the 5′-cap is linked to the 5′-terminus via a 5-5-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Cellular immunity/cellular immune response: Cellular immunity relates typically to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface. Such cells may be virus-infected or infected with intracellular bacteria, or cancer cells displaying tumor antigens. Further characteristics may be activation of macrophages and natural killer cells, enabling them to destroy pathogens and stimulation of cells to secrete a variety of cytokines that influence the function of other cells involved in adaptive immune responses and innate immune responses.

The term “derived from” as used throughout the present specification in the context of a nucleic acid, i.e. for a nucleic acid “derived from” (another) nucleic acid, means that the nucleic acid, which is derived from (another) nucleic acid, shares at least 50%, preferably at least 60%, preferably at least 70%, more preferably at least 75%, more preferably at least 80%, more preferably at least 85%, even more preferably at least 90%, even more preferably at least 95%, and particularly preferably at least 98% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the thymidines (T) by uracils (U) throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. It goes without saying that such modifications are preferred, which do not impair RNA stability, e.g. in comparison to the nucleic acid from which it is derived.

DNA: DNA is the usual abbreviation for deoxy-ribonucleic acid. It is a nucleic acid molecule, i.e. a polymer consisting of nucleotides. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerise by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing.

Epitope: (also called “antigen determinant”) can be distinguished in T cell epitopes and B cell epitopes. T cell epitopes or parts of the proteins in the context of the present invention may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens as defined herein, preferably having 5 to 15 amino acids, more preferably having 5 to 12 amino acids, even more preferably having 6 to 9 amino acids, which may be recognized by antibodies, i.e. in their native form.

Such epitopes of proteins or peptides may furthermore be selected from any of the herein mentioned variants of such proteins or peptides. In this context antigenic determinants can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment of a sequence: A fragment of a sequence may typically be a shorter portion of a full-length sequence of e.g. a nucleic acid molecule or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 5%, 10%, 20%, preferably at least 30%, more preferably at least 40%, more preferably at least 50%, even more preferably at least 60%, even more preferably at least 70%, and most preferably at least 80% of the total (i.e. full-length) molecule from which the fragment is derived.

G/C modified: A G/C-modified nucleic acid may typically be a nucleic acid, preferably an artificial nucleic acid molecule as defined herein, based on a modified wild-type sequence comprising a preferably increased number of guanosine and/or cytosine nucleotides as compared to the wild-type sequence. Such an increased number may be generated by substitution of codons containing adenosine or thymidine nucleotides by codons containing guanosine or cytosine nucleotides. If the enriched G/C content occurs in a coding region of DNA or RNA, it makes use of the degeneracy of the genetic code. Accordingly, the codon substitutions preferably do not alter the encoded amino acid residues, but exclusively increase the G/C content of the nucleic acid molecule.

Gene therapy: Gene therapy may typically be understood to mean a treatment of a patient's body or isolated elements of a patient's body, for example isolated tissues/cells, by nucleic acids encoding a peptide or protein. It typically may comprise at least one of the steps of a) administration of a nucleic acid, preferably an artificial nucleic acid molecule as defined herein, directly to the patient—by whatever administration route—or in vitro to isolated cells/tissues of the patient, which results in transfection of the patient's cells either in vivo/ex vivo or in vitro; b) transcription and/or translation of the introduced nucleic acid molecule; and optionally c) re-administration of isolated, transfected cells to the patient, if the nucleic acid has not been administered directly to the patient.

Genetic vaccination: Genetic vaccination may typically be understood to be vaccination by administration of a nucleic acid molecule encoding an antigen or an immunogen or fragments thereof. The nucleic acid molecule may be administered to a subject's body or to isolated cells of a subject. Upon transfection of certain cells of the body or upon transfection of the isolated cells, the antigen or immunogen may be expressed by those cells and subsequently presented to the immune system, eliciting an adaptive, i.e. antigen-specific immune response. Accordingly, genetic vaccination typically comprises at least one of the steps of a) administration of a nucleic acid, preferably an artificial nucleic acid molecule as defined herein, to a subject, preferably a patient, or to isolated cells of a subject, preferably a patient, which usually results in transfection of the subject's cells either in vivo or in vitro; b) transcription and/or translation of the introduced nucleic acid molecule; and optionally c) re-administration of isolated, transfected cells to the subject, preferably the patient, if the nucleic acid has not been administered directly to the patient.

Heterologous sequence: Two sequences are typically understood to be ‘heterologous’ if they are not derivable from the same gene. I.e., although heterologous sequences may be derivable from the same organism, they naturally (in nature) do not occur in the same nucleic acid molecule, such as in the same mRNA.

Humoral immunity/humoral immune response: Humoral immunity refers typically to antibody production and optionally to accessory processes accompanying antibody production. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Immunogen: In the context of the present invention an immunogen may be typically understood to be a compound that is able to stimulate an immune response. Preferably, an immunogen is a peptide, polypeptide, or protein. In a particularly preferred embodiment, an immunogen in the sense of the present invention is the product of translation of a provided nucleic acid molecule, preferably an artificial nucleic acid molecule as defined herein. Typically, an immunogen elicits at least an adaptive immune response.

Immunostimulatory composition: In the context of the invention, an immunostimulatory composition may be typically understood to be a composition containing at least one component which is able to induce an immune response or from which a component which is able to induce an immune response is derivable. Such immune response may be preferably an innate immune response or a combination of an adaptive and an innate immune response. Preferably, an immunostimulatory composition in the context of the invention contains at least one artificial nucleic acid molecule, more preferably an RNA, for example an mRNA molecule. The immunostimulatory component, such as the mRNA may be complexed with a suitable carrier. Thus, the immunostimulatory composition may comprise an mRNA/carrier-complex. Furthermore, the immunostimulatory composition may comprise an adjuvant and/or a suitable vehicle for the immunostimulatory component, such as the mRNA.

Immune response: An immune response may typically be a specific reaction of the adaptive immune system to a particular antigen (so called specific or adaptive immune response) or an unspecific reaction of the innate immune system (so called unspecific or innate immune response), or a combination thereof.

Immune system: The immune system may protect organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Immunostimulatory RNA: An immunostimulatory RNA (isRNA) in the context of the invention may typically be an RNA that is able to induce an innate immune response. It usually does not have an open reading frame and thus does not provide a peptide-antigen or immunogen but elicits an immune response e.g. by binding to a specific kind of Toll-like-receptor (TLR) or other suitable receptors. However, of course also mRNAs having an open reading frame and coding for a peptide/protein may induce an innate immune response and, thus, may be immunostimulatory RNAs.

Innate immune system: The innate immune system, also known as non-specific (or unspecific) immune system, typically comprises the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g., activated by ligands of Toll-like receptors (TLRs) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent. The pharmaceutical composition according to the present invention may comprise one or more such substances. Typically, a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system; and/or acting as a physical and chemical barrier to infectious agents.

Cloning site: A cloning site is typically understood to be a segment of a nucleic acid molecule, which is suitable for insertion of a nucleic acid sequence, e.g., a nucleic acid sequence comprising an open reading frame. Insertion may be performed by any molecular biological method known to the one skilled in the art, e.g. by restriction and ligation. A cloning site typically comprises one or more restriction enzyme recognition sites (restriction sites). These one or more restrictions sites may be recognized by restriction enzymes which cleave the DNA at these sites. A cloning site which comprises more than one restriction site may also be termed a multiple cloning site (MCS) or a polylinker.

Nucleic acid molecule: A nucleic acid molecule is a molecule comprising, preferably consisting of nucleic acid components. The term nucleic acid molecule preferably refers to DNA or RNA molecules. It is preferably used synonymous with the term “polynucleotide”. Preferably, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

Open reading frame: An open reading frame (ORF) in the context of the invention may typically be a sequence of several nucleotide triplets which may be translated into a peptide or protein. An open reading frame preferably contains a start codon, i.e. a combination of three subsequent nucleotides coding usually for the amino acid methionine (ATG), at its 5′-end and a subsequent region which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present invention is preferably a nucleotide sequence, consisting of a number of nucleotides that may be divided by three, which starts with a start codon (e.g. ATG) and which preferably terminates with a stop codon (e.g., TAA, TGA, or TAG). The open reading frame may be isolated or it may be incorporated in a longer nucleic acid sequence, for example in a vector or an mRNA. An open reading frame may also be termed “protein coding region”.

Peptide: A peptide or polypeptide is typically a polymer of amino acid monomers, linked by peptide bonds. It typically contains less than 50 monomer units. Nevertheless, the term peptide is not a disclaimer for molecules having more than 50 monomer units. Long peptides are also called polypeptides, typically having between 50 and 600 monomeric units.

Pharmaceutically effective amount: A pharmaceutically effective amount in the context of the invention is typically understood to be an amount that is sufficient to induce a pharmaceutical effect, such as an immune response, altering a pathological level of an expressed peptide or protein, or substituting a lacking gene product, e.g., in case of a pathological situation.

Protein A protein typically comprises one or more peptides or polypeptides. A protein is typically folded into 3-dimensional form, which may be required for to protein to exert its biological function.

Poly(A) sequence: A poly(A) sequence, also called poly(A) tail or 3′-poly(A) tail, is typically understood to be a sequence of adenosine nucleotides, e.g., of up to about 400 adenosine nucleotides, e.g. from about 20 to about 400, preferably from about 50 to about 400, more preferably from about 50 to about 300, even more preferably from about 50 to about 250, most preferably from about 60 to about 250 adenosine nucleotides. A poly(A) sequence is typically located at the 3′end of an mRNA. In the context of the present invention, a poly(A) sequence may be located within an mRNA or any other nucleic acid molecule, such as, e.g., in a vector, for example, in a vector serving as template for the generation of an RNA, preferably an mRNA, e.g., by transcription of the vector.

Polyadenylation: Polyadenylation is typically understood to be the addition of a poly(A) sequence to a nucleic acid molecule, such as an RNA molecule, e.g. to a premature mRNA. Polyadenylation may be induced by a so called polyadenylation signal. This signal is preferably located within a stretch of nucleotides at the 3′-end of a nucleic acid molecule, such as an RNA molecule, to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation.

Restriction site: A restriction site, also termed restriction enzyme recognition site, is a nucleotide sequence recognized by a restriction enzyme. A restriction site is typically a short, preferably palindromic nucleotide sequence, e.g. a sequence comprising 4 to 8 nucleotides. A restriction site is preferably specifically recognized by a restriction enzyme. The restriction enzyme typically cleaves a nucleotide sequence comprising a restriction site at this site. In a double-stranded nucleotide sequence, such as a double-stranded DNA sequence, the restriction enzyme typically cuts both strands of the nucleotide sequence.

RNA, mRNA: RNA is the usual abbreviation for ribonucleic-acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. Usually RNA may be obtainable by transcription of a DNA-sequence, e.g., inside a cell. In eukaryotic cells, transcription is typically performed inside the nucleus or the mitochondria. Typically, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino-acid sequence of a particular peptide or protein. Typically, a mature mRNA comprises a 5′-cap, a 5′-UTR, an open reading frame, a 3′-UTR and a poly(A) sequence. Aside from messenger RNA, several non-coding types of RNA exist which may be involved in regulation of transcription and/or translation.

Sequence of a nucleic acid molecule: The sequence of a nucleic acid molecule is typically understood to be the particular and individual order, i.e. the succession of its nucleotides. The sequence of a protein or peptide is typically understood to be the order, i.e. the succession of its amino acids.

Sequence identity: Two or more sequences are identical if they exhibit the same length and order of nucleotides or amino acids. The percentage of identity typically describes the extent to which two sequences are identical, i.e. it typically describes the percentage of nucleotides that correspond in their sequence position with identical nucleotides of a reference-sequence. For determination of the degree of identity, the sequences to be compared are considered to exhibit the same length, i.e. the length of the longest sequence of the sequences to be compared. This means that a first sequence consisting of 8 nucleotides is 80% identical to a second sequence consisting of 10 nucleotides comprising the first sequence. In other words, in the context of the present invention, identity of sequences preferably relates to the percentage of nucleotides of a sequence which have the same position in two or more sequences having the same length. Gaps are usually regarded as non-identical positions, irrespective of their actual position in an alignment.

Stabilized nucleic acid molecule: A stabilized nucleic acid molecule is a nucleic acid molecule, preferably a DNA or RNA molecule that is modified such, that it is more stable to disintegration or degradation, e.g., by environmental factors or enzymatic digest, such as by an exo- or endonuclease degradation, than the nucleic acid molecule without the modification. Preferably, a stabilized nucleic acid molecule in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

Transfection: The term “transfection” refers to the introduction of nucleic acid molecules, such as DNA or RNA (e.g. mRNA) molecules, into cells, preferably into eukaryotic cells. In the context of the present invention, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. Preferably, the introduction is non-viral.

Vaccine: A vaccine is typically understood to be a prophylactic or therapeutic material providing at least one antigen, preferably an immunogen. The antigen or immunogen may be derived from any material that is suitable for vaccination. For example, the antigen or immunogen may be derived from a pathogen, such as from bacteria or virus particles etc., or from a tumor or cancerous tissue. The antigen or immunogen stimulates the body's adaptive immune system to provide an adaptive immune response.

Vector: The term “vector” refers to a nucleic acid molecule, preferably to an artificial nucleic acid molecule. A vector in the context of the present invention is suitable for incorporating or harboring a desired nucleic acid sequence, such as a nucleic acid sequence comprising an open reading frame. Such vectors may be storage vectors, expression vectors, cloning vectors, transfer vectors etc. A storage vector is a vector which allows the convenient storage of a nucleic acid molecule, for example, of an mRNA molecule. Thus, the vector may comprise a sequence corresponding, e.g., to a desired mRNA sequence or a part thereof, such as a sequence corresponding to the open reading frame and the 3′-UTR and/or the 5′-UTR of an mRNA. An expression vector may be used for production of expression products such as RNA, e.g. mRNA, or peptides, polypeptides or proteins. For example, an expression vector may comprise sequences needed for transcription of a sequence stretch of the vector, such as a promoter sequence, e.g. an RNA polymerase promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. Preferably, a vector is a DNA molecule. Preferably, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector.

Vehicle: A vehicle is typically understood to be a material that is suitable for storing, transporting, and/or administering a compound, such as a pharmaceutically active compound. For example, it may be a physiologically acceptable liquid which is suitable for storing, transporting, and/or administering a pharmaceutically active compound.

3′-untranslated region (3′-UTR): Generally, the term “3′-UTR” refers to a part of the artificial nucleic acid molecule, which is located 3′ (i.e. “downstream”) of an open reading frame and which is not translated into protein. Typically, a 3′-UTR is the part of an mRNA which is located between the protein coding region (open reading frame (ORF) or coding sequence (CDS)) and the poly(A) sequence of the mRNA. In the context of the invention, the term 3′-UTR may also comprise elements, which are not encoded in the template, from which an RNA is transcribed, but which are added after transcription during maturation, e.g. a poly(A) sequence. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′capping, splicing the pre-mature mRNA to excize optional introns and modifications of the 3′-end, such as polyadenylation of the 3′-end of the pre-mature mRNA and optional endo-/or exonuclease cleavages etc. In the context of the present invention, a 3′-UTR corresponds to the sequence of a mature mRNA which is located between the the stop codon of the protein coding region, preferably immediately 3′ to the stop codon of the protein coding region, and the poly(A) sequence of the mRNA. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence (both sense and antisense strand and both mature and immature) of the 3′-UTR. Preferably, the 3′UTRs have a length of more than 20, 30, 40 or 50 nucleotides.

5′-untranslated region (5′-UTR): Generally, the term “5′-UTR” refers to a part of the artificial nucleic acid molecule, which is located 5′ (i.e. “upstream”) of an open reading frame and which is not translated into protein. A 5′-UTR is typically understood to be a particular section of messenger RNA (mRNA), which is located 5′ of the open reading frame of the mRNA. Typically, the 5′-UTR starts with the transcriptional start site and ends one nucleotide before the start codon of the open reading frame. Preferably, the 5′UTRs have a length of more than 20, 30, 40 or 50 nucleotides. The 5′-UTR may comprise elements for controlling gene expression, also called regulatory elements. Such regulatory elements may be, for example, ribosomal binding sites. The 5′-UTR may be posttranscriptionally modified, for example by addition of a 5′-CAP. A 5′-UTR of the mRNA is not translated into an amino acid sequence. The 5′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′capping, splicing the pre-mature mRNA to excize optional introns and modifications of the 3′-end, such as polyadenylation of the 3′-end of the pre-mature mRNA and optional endo-/or exonuclease cleavages etc. In the context of the present invention, a 5′-UTR corresponds to the sequence of a mature mRNA which is located between the start codon and, for example, the 5′-CAP. Preferably, the 5′-UTR corresponds to the sequence which extends from a nucleotide located 3′ to the 5′-CAP, more preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene” is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence (both sense and antisense strand and both mature and immature) of the 5′-UTR.

5′Terminal Oligopyrimidine Tract (TOP): The 5′terminal oligopyrimidine tract (TOP) is typically a stretch of pyrimidine nucleotides located in the 5′ terminal region of a nucleic acid molecule, such as the 5′ terminal region of certain mRNA molecules or the 5′ terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′ TOP ends one nucleotide 5′ to the first purine nucleotide located downstream of the TOP. Messenger RNA that contains a 5′terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′end with a cytosine nucleotide. In TOP genes and TOP mRNAs, the TOP-motif preferably starts at its 5′end with the transcriptional start site and ends one nucleotide 5′ to the first purin residue in said gene or mRNA. A TOP motif in the sense of the present invention is preferably located at the 5′end of a sequence which represents a 5′-UTR or at the 5′end of a sequence which codes for a 5′-UTR. Thus, preferably, a stretch of 3 or more pyrimidine nucleotides is called “TOP motif” in the sense of the present invention if this stretch is located at the 5′end of a respective sequence, such as the artificial nucleic acid molecule, the 5′-UTR element of the artificial nucleic acid molecule, or the nucleic acid sequence which is derived from the 5′-UTR of a TOP gene as described herein. In other words, a stretch of 3 or more pyrimidine nucleotides, which is not located at the 5′-end of a 5′-UTR or a 5′-UTR element but anywhere within a 5′-UTR or a 5′-UTR element, is preferably not referred to as “TOP motif”.

TOP gene: TOP genes are typically characterised by the presence of a 5′ terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-CAP to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID Nos. 1-1363 of the patent application WO2013/143700, whose disclosure is incorporated herewith by reference. In this context a particularly preferred fragment of a 5′-UTR of a TOP gene is a 5′-UTR of a TOP gene lacking the 5′TOP motif. The terms “5′-UTR of a TOP gene” or “5′-TOP UTR” preferably refer to the 5′-UTR of a naturally occurring TOP gene.

In a first aspect, the present invention relates to an artificial nucleic acid molecule comprising
a. at least one open reading frame (ORF); and
b. at least one 3′-untranslated region element (3′-UTR element) and/or at least one 5′-untranslated region element (5′-UTR element), wherein the at least one 3′-UTR element and/or the at least one 5′-UTR element prolongs and/or increases protein production from said artificial nucleic acid molecule and wherein the at least one 3′-UTR element and/or the at least one 5′-UTR element is derived from a stable mRNA.

Preferably, the artificial nucleic acid molecule according to the present invention does not comprise a 3′-UTR (element) and/or a 5′-UTR (element) of ribosomal protein S6, of RPL36AL, of rps16 or of ribosomal protein L9. More preferably, the artificial nucleic acid molecule according to the present invention does not comprise a 3′-UTR (element) and/or a 5′-UTR (element) of ribosomal protein S6, of RPL36AL, of rps16 or of ribosomal protein L9 and the open reading frame of the artificial nucleic acid molecule according to the present invention does not code for a GFP protein. Even more preferably, the artificial nucleic acid molecule according to the present invention does not comprise a 3′-UTR (element) and/or a 5′-UTR (element) of ribosomal protein S6, of RPL36AL, of rps16 or of ribosomal protein L9 and the open reading frame of the artificial nucleic acid molecule according to the present invention does not code for a reporter protein, e.g., selected from the group consisting of globin proteins (particularly beta-globin), luciferase protein, GFP proteins, glucurinodase proteins (particularly beta-glucurinodase) or variants thereof, for example, variants exhibiting at least 70% sequence identity to a globin protein, a luciferase protein, a GFP protein, or a glucurinodase protein.

The term “3′-UTR element” refers to a nucleic acid sequence which comprises or consists of a nucleic acid sequence that is derived from a 3′-UTR or from a variant or a fragment of a 3′-UTR. A “3′-UTR element” preferably refers to a nucleic acid sequence which is comprised by a 3′-UTR of an artificial nucleic acid sequence, such as an artificial mRNA. Accordingly, in the sense of the present invention, preferably, a 3′-UTR element may be comprised by the 3′-UTR of an mRNA, preferably of an artificial mRNA, or a 3′-UTR element may be comprised by the 3′-UTR of the respective transcription template. Preferably, a 3′-UTR element is a nucleic acid sequence which corresponds to the 3′-UTR of an mRNA, preferably to the 3′-UTR of an artificial mRNA, such as an mRNA obtained by transcription of a genetically engineered vector construct. Preferably, a 3′-UTR element in the sense of the present invention functions as a 3′-UTR or codes for a nucleotide sequence that fulfils the function of a 3′-UTR.

Accordingly, the term “5′-UTR element” refers to a nucleic acid sequence which comprises or consists of a nucleic acid sequence that is derived from a 5′-UTR or from a variant or a fragment of a 5′-UTR. A “5′-UTR element” preferably refers to a nucleic acid sequence which is comprised by a 5′-UTR of an artificial nucleic acid sequence, such as an artificial mRNA. Accordingly, in the sense of the present invention, preferably, a 5′-UTR element may be comprised by the 5′-UTR of an mRNA, preferably of an artificial mRNA, or a 5′-UTR element may be comprised by the 5′-UTR of the respective transcription template. Preferably, a 5′-UTR element is a nucleic acid sequence which corresponds to the 5′-UTR of an mRNA, preferably to the 5′-UTR of an artificial mRNA, such as an mRNA obtained by transcription of a genetically engineered vector construct. Preferably, a 5′-UTR element in the sense of the present invention functions as a 5′-UTR or codes for a nucleotide sequence that fulfils the function of a 5′-UTR.

The 3′-UTR element and/or the 5′-UTR element in the artificial nucleic acid molecule according to the present invention prolongs and/or increases protein production from said artificial nucleic acid molecule. Thus, the artificial nucleic acid molecule according to the present invention may in particular comprise:
a 3′-UTR element which increases protein production from said artificial nucleic acid molecule,
a 3′-UTR element which prolongs protein production from said artificial nucleic acid molecule,
a 3′-UTR element which increases and prolongs protein production from said artificial nucleic acid molecule,
a 5′-UTR element which increases protein production from said artificial nucleic acid molecule,
a 5′-UTR element which prolongs protein production from said artificial nucleic acid molecule,
a 5′-UTR element which increases and prolongs protein production from said artificial nucleic acid molecule,
a 3′-UTR element which increases protein production from said artificial nucleic acid molecule and a 5′-UTR element which increases protein production from said artificial nucleic acid molecule,
a 3′-UTR element which increases protein production from said artificial nucleic acid molecule and a 5′-UTR element which prolongs protein production from said artificial nucleic acid molecule,
a 3′-UTR element which increases protein production from said artificial nucleic acid molecule and a 5′-UTR element which increases and prolongs protein production from said artificial nucleic acid molecule,
a 3′-UTR element which prolongs protein production from said artificial nucleic acid molecule and a 5′-UTR element which increases protein production from said artificial nucleic acid molecule,
a 3′-UTR element which prolongs protein production from said artificial nucleic acid molecule and a 5′-UTR element which prolongs protein production from said artificial nucleic acid molecule,
a 3′-UTR element which prolongs protein production from said artificial nucleic acid molecule and a 5′-UTR element which increases and prolongs protein production from said artificial nucleic acid molecule,
a 3′-UTR element which increases and prolongs protein production from said artificial nucleic acid molecule and a 5′-UTR element which increases protein production from said artificial nucleic acid molecule,
a 3′-UTR element which increases and prolongs protein production from said artificial nucleic acid molecule and a 5′-UTR element which prolongs protein production from said artificial nucleic acid molecule, or
a 3′-UTR element which increases and prolongs protein production from said artificial nucleic acid molecule and a 5′-UTR element which increases and prolongs protein production from said artificial nucleic acid molecule.

Preferably, the artificial nucleic acid molecule according to the present invention comprises a 3′-UTR element which prolongs protein production from said artificial nucleic acid molecule and/or a 5′-UTR element which increases protein production from said artificial nucleic acid molecule.

Preferably, the artificial nucleic acid molecule according to the present invention comprises at least one 3′-UTR element and at least one 5′-UTR element, i.e. at least one 3′-UTR element which prolongs and/or increases protein production from said artificial nucleic acid molecule and which is derived from a stable mRNA and at least one 5′-UTR element which prolongs and/or increases protein production from said artificial nucleic acid molecule and which is derived from a stable mRNA.

“Prolonging and/or increasing protein production from said artificial nucleic acid molecule” in general refers to the amount of protein produced from the artificial nucleic acid molecule according to the present invention with the respective 3′-UTR element and/or the 5′-UTR element in comparison to the amount of protein produced from a respective reference nucleic acid lacking a 3′-UTR and/or a 5′-UTR or comprising a reference 3′-UTR and/or a reference 5′-UTR, such as a 3′-UTR and/or a 5′-UTR naturally occurring in combination with the ORF.

In particular, the at least one 3′-UTR element and/or the 5′-UTR element of the artificial nucleic acid molecule according to the present invention prolongs protein production from the artificial nucleic acid molecule according to the present invention, e.g. from an mRNA according to the present invention, compared to a respective nucleic acid lacking a 3′-UTR and/or 5′-UTR or comprising a reference 3′-UTR and/or 5′-UTR, such as a 3′- and/or 5′-UTR naturally occurring in combination with the ORF.

In particular, the at least one 3′-UTR element and/or 5′-UTR element of the artificial nucleic acid molecule according to the present invention increases protein production, in particular the protein expression and/or total protein production, from the artificial nucleic acid molecule according to the present invention, e.g. from an mRNA according to the present invention, compared to a respective nucleic acid lacking a 3′- and/or 5′-UTR or comprising a reference 3′- and/or 5′-UTR, such as a 3′- and/or 5′-UTR naturally occurring in combination with the ORF.

Preferably, the at least one 3′-UTR element and/or the at least one 5′-UTR element of the artificial nucleic acid molecule according to the present invention do not negatively influence translational efficiency of a nucleic acid compared to the translational efficiency of a respective nucleic acid lacking a 3′-UTR and/or a 5′-UTR or comprising a reference 3′-UTR and/or a reference 5′-UTR, such as a 3′-UTR and/or a 5′-UTR naturally occurring in combination with the ORF. Even more preferably, the translation efficiency is enhanced by the 3′-UTR and/or a 5′-UTR in comparison to the translation efficiency of the protein encoded by the respective ORF in its natural context.

The term “respective nucleic acid molecule” or “reference nucleic acid molecule” as used herein means that—apart from the different 3′-UTRs and/or 5′-UTRs—the reference nucleic acid molecule is comparable, preferably identical, to the inventive artificial nucleic acid molecule comprising the 3′-UTR element and/or the 5′-UTR element.

In order to assess the protein production in vivo or in vitro as defined herein (i.e. in vitro referring to (“living”) cells and/or tissue, including tissue of a living subject; cells include in particular cell lines, primary cells, cells in tissue or subjects, preferred are mammalian cells, e.g. human cells and mouse cells and particularly preferred are the human cell lines HeLa, and U-937 and the mouse cell lines NIH3T3, JAWSII and L929, furthermore primary cells are particularly preferred, in particular preferred embodiments human dermal fibroblasts (HDF)) by the inventive artificial nucleic acid molecule, the expression of the encoded protein is determined following injection/transfection of the inventive artificial nucleic acid molecule into target cells/tissue and compared to the protein expression induced by the reference nucleic acid. Quantitative methods for determining protein expression are known in the art (e.g. Western-Blot, FACS, ELISA, mass spectrometry). Particularly useful in this context is the determination of the expression of reporter proteins like luciferase, Green fluorescent protein (GFP), or secreted alkaline phosphatase (SEAP). Thus, an artificial nucleic acid according to the invention or a reference nucleic acid is introduced into the target tissue or cell, e.g. via transfection or injection, preferably in a mammalian expression system, such as in mammalian cells, e.g. in HeLa or HDF cells. Several hours or several days (e.g. 6, 12, 24, 48 or 72 hours) post initiation of expression or post introduction of the nucleic acid molecule, a target cell sample is collected and measured via FACS and/or lysed. Afterwards the lysates can be used to detect the expressed protein (and thus determine the efficiency of protein expression) using several methods, e.g. Western-Blot, FACS, ELISA, mass spectrometry or by fluorescence or luminescence measurement.

Therefore, if the protein expression from an artificial nucleic acid molecule according to the invention is compared to the protein expression from a reference nucleic acid molecule at a specific point in time (e.g. 6, 12, 24, 48 or 72 hours post initiation of expression or post introduction of the nucleic acid molecule), both nucleic acid molecules are introduced separately into target tissue/cells, a sample from the tissue/cells is collected after a specific point in time, protein lysates are prepared according to the particular protocol adjusted to the particular detection method (e.g. Western Blot, ELISA, fluorescence or luminescence measurement, etc. as known in the art) and the protein is detected by the chosen detection method. As an alternative to the measurement of expressed protein amounts in cell lysates—or, in addition to the measurement of protein amounts in cell lysates prior to lysis of the collected cells or using an aliquot in parallel—protein amounts may also be determined by using FACS analysis.

The term “prolonging protein production” from an artificial nucleic acid molecule such as an artificial mRNA preferably means that the protein production from the artificial nucleic acid molecule such as the artificial mRNA is prolonged compared to the protein production from a reference nucleic acid molecule such as a reference mRNA, e.g. comprising a reference 3′- and/or 5′-UTR or lacking a 3′- and/or 5′-UTR, preferably in a mammalian expression system, such as in HeLa or HDF cells. Thus, protein produced from the artificial nucleic acid molecule such as the artificial mRNA is observable for a longer period of time than what may be seen for a protein produced from a reference nucleic acid molecule. In other words, the amount of protein produced from the artificial nucleic acid molecule such as the artificial mRNA measured at a later point in time, e.g. 48 hours or 72 hours after transfection, is larger than the amount of protein produced from a reference nucleic acid molecule such as a reference mRNA at a corresponding later point in time. Such a “later point in time” may be, for example, any time beyond 24 hours post initiation of expression, such as post transfection of the nucleic acid molecule, e.g. 36, 48, 60, 72, 96 hours post initiation of expression, i.e. after transfection. Moreover, for the same nucleic acid, the amount of protein produced at a later point in time may be normalized to the amount produced an earlier (reference) point in time, for example the amount of protein at a later point in time may be expressed as percentage of the amount of protein at 24 h after transfection.

Preferably, this effect of prolonging protein production is determined by (i) measuring protein amounts, e.g. obtained by expression of an encoded reporter protein such as luciferase, preferably in a mammalian expression system such as in HeLa or HDF cells, over time, (ii) determining the amount of protein observed at a “reference” point in time t₁, for example t₁=24 h after transfection, and setting this protein amount to 100%, (iii) determining the amount of protein observed at one or more later points in time t₂, t₃, etc., for example t₂=48 h and t₃=72 h after transfection, and calculating the relative amount of protein observed at a later point in time as a percentage of the protein amount at a point in time t₁. For example, a protein which is expressed at t₁ in an amount of “80”, at t₂ in an amount of “20”, and at t₃ in an amount of “10”, the relative amount of protein at t₂ would be 25%, and at t₃ 12.5%. These relative amounts at a later point in time may then be compared in a step (iv) to relative protein amounts for the corresponding points in time for a nucleic acid molecule lacking a 3′- and/or 5′-UTR, respectively, or comprising a reference 3′- and/or 5′-UTR, respectively. By comparing the relative protein amount produced from the artificial nucleic acid molecule according to the present invention to the relative protein amount produced from the reference nucleic acid molecule, i.e. the nucleic acid molecule lacking a 3′- and/or 5′-UTR, respectively, or comprising a reference 3′- and/or 5′-UTR, respectively, a factor may be determined by which the protein production from the artificial nucleic acid molecule according to the present invention is prolonged compared to the protein production from the reference nucleic acid molecule.

Preferably, the at least one 3′- and/or 5′-UTR element in the artificial nucleic acid molecule according to the invention prolongs protein production from said artificial nucleic acid molecule at least 1.2 fold, preferably at least 1.5 fold, more preferably at least 2 fold, even more preferably at least 2.5 fold, compared to the protein production from a reference nucleic acid molecule lacking 3′- and/or 5′-UTR, respectively, or comprising a reference 3′- and/or 5′-UTR, respectively. In other words, the (relative) amount of protein produced from in the artificial nucleic acid molecule according to the invention at a certain later point in time as described above is increased by a factor of at least 1.2, preferably at least 1.5, more preferably at least 2, even more preferably at least 2.5, compared to the (relative) amount of protein produced from a reference nucleic acid molecule, which is e.g. lacking a 3′- and/or 5′-UTR, respectively, or comprising a reference 3′- and/or 5′-UTR, respectively, for the same later point in time.

Alternatively, the effect of prolonging protein production may also be determined by (i) measuring protein amounts, e.g. obtained by expression of an encoded reporter protein such as luciferase, preferably in a mammalian expression system such as in HeLa or HDF cells, over time, (ii) determining the point in time at which the protein amount undercuts the amount of protein observed, e.g., at 1, 2, 3, 4, 5, or 6 hours post initiation of expression, e.g. 1, 2, 3, 4, 5, or 6 hours post transfection of the artificial nucleic acid molecule, and (iii) comparing the point in time at which the protein amount undercuts the protein amount observed at 1, 2, 3, 4, 5, or 6 hours post initiation of expression to said point in time determined for a nucleic acid molecule lacking a 3′- and/or 5′-UTR, respectively, or comprising a reference 3′- and/or 5′-UTR, respectively.

For example, the protein production from the artificial nucleic acid molecule such as the artificial mRNA—in an amount which is at least the amount observed in the initial phase of expression, such as 1, 2, 3, 4, 5, or 6 hours post initiation of expression, such as post transfection of the nucleic acid molecule—is prolonged by at least about 5 hours, preferably by at least about 10 hours, more preferably by at least about 24 hours compared to the protein production from a reference nucleic acid molecule, such as a reference mRNA, in a mammalian expression system, such as in mammalian cells, e.g. in HeLa or HDF cells. Thus, the artificial nucleic acid molecule according to the present invention preferably allows for prolonged protein production in an amount which is at least the amount observed in the initial phase of expression, such as 1, 2, 3, 4, 5, or 6 hours post initiation of expression, such as post transfection, by at least about 5 hours, preferably by at least about 10 hours, more preferably by at least about 24 hours compared to a reference nucleic acid molecule lacking a 3′- and/or 5′-UTR, respectively, or comprising a reference 3′- and/or 5′-UTR, respectively.

In preferred embodiments, the period of protein production from the artificial nucleic acid molecule according to the present invention is extended at least 1.2 fold, preferably at least 1.5 fold, more preferably at least 2 fold, even more preferably at least 2.5 fold, compared to the protein production from a reference nucleic acid molecule lacking a 3′- and/or 5′-UTR, respectively, or comprising a reference 3′- and/or 5′-UTR, respectively.

Preferably, this prolonging effect on protein production is achieved, while the total amount of protein produced from the artificial nucleic acid molecule according to the present invention, e.g. within a time span of 48 or 72 hours, corresponds at least to the amount of protein produced from a reference nucleic acid molecule lacking a 3′- and/or 5′-UTR, respectively, or comprising a reference 3′- and/or 5′-UTR, respectively, such as a 3′-UTR and/or 5′-UTR naturally occurring with the ORF of the artificial nucleic acid molecule. Thus, the present invention provides an artificial nucleic acid molecule which allows for prolonged protein production in a mammalian expression system, such as in mammalian cells, e.g. in HeLa or HDF cells, as specified above, wherein the total amount of protein produced from said artificial nucleic acid molecule, e.g. within a time span of 48 or 72 hours, is at least the total amount of protein produced, e.g. within said time span, from a reference nucleic acid molecule lacking a 3′- and/or 5′-UTR, respectively, or comprising a reference 3′- and/or 5′-UTR, respectively, such as a 3′- and/or 5′-UTR naturally occurring with the ORF of the artificial nucleic acid molecule.

Moreover, the term “prolonged protein expression” also includes “stabilized protein expression”, whereby “stabilized protein expression” preferably means that there is more uniform protein production from the artificial nucleic acid molecule according to the present invention over a predetermined period of time, such as over 24 hours, more preferably over 48 hours, even more preferably over 72 hours, when compared to a reference nucleic acid molecule, for example, an mRNA comprising a reference 3′- and/or 5′-UTR, respectively, or lacking a 3′- and/or 5′-UTR, respectively.

Accordingly, the level of protein production, e.g. in a mammalian system, from the artificial nucleic acid molecule comprising a 3′- and/or 5′-UTR element according to the present invention, e.g. from an mRNA according to the present invention, preferably does not drop to the extent observed for a reference nucleic acid molecule, such as a reference mRNA as described above. To assess to which extent the protein production from a specific nucleic acid molecule drops, for example, the amount of a protein (encoded by the respective ORF) observed 24 hours after initiation of expression, e.g. 24 hours post transfection of the artificial nucleic acid molecule according to the present invention into a cell, such as a mammalian cell, may be compared to the amount of protein observed 48 hours after initiation of expression, e.g. 48 hours post transfection. Thus, the ratio of the amount of protein encoded by the ORF of the artificial nucleic acid molecule according to the present invention, such as the amount of a reporter protein, e.g., luciferase, observed at a later point in time, e.g. 48 hours, post initiation of expression, e.g. post transfection, to the amount of protein observed at an earlier point in time, e.g. 24 hours, post initiation of expression, e.g. post transfection, is preferably higher than the corresponding ratio (including the same points in time) for a reference nucleic acid molecule comprising a reference 3′- and/or 5′-UTR, respectively, or lacking a 3′- and/or 5′-UTR, respectively.

Preferably, the ratio of the amount of protein encoded by the ORF of the artificial nucleic acid molecule according to the present invention, such as the amount of a reporter protein, e.g., luciferase, observed at a later point in time, e.g. 48 hours, post initiation of expression, e.g. post transfection, to the amount of protein observed at an earlier point in time, e.g. 24 hours, post initiation of expression, e.g. post transfection, is preferably at least 0.2, more preferably at least about 0.3, even more preferably at least about 0.4, even more preferably at least about 0.5, and particularly preferably at least about 0.7. For a respective reference nucleic acid molecule, e.g. an mRNA comprising a reference 3′- and/or 5′-UTR, respectively, or lacking a 3′- and/or 5′-UTR, respectively, said ratio may be, for example between about 0.05 and about 0.35.

Thus, the present invention provides an artificial nucleic acid molecule comprising an ORF and a 3′- and/or 5′-UTR element as described above, wherein the ratio of the protein amount, e.g. the amount of luciferase, observed 48 hours after initiation of expression to the protein amount observed 24 hours after initiation of expression, preferably in a mammalian expression system, such as in mammalian cells, e.g. in HDF cells or in HeLa cells, is preferably at least 0.2, more preferably at least about 0.3, more preferably at least about 0.4, even more preferably at least about 0.5, even more preferably at least about 0.6, and particularly preferably at least about 0.7. Thereby, preferably the total amount of protein produced from said artificial nucleic acid molecule, e.g. within a time span of 48 hours, corresponds at least to the total amount of protein produced, e.g. within said time span, from a reference nucleic acid molecule lacking a 3′- and/or 5′-UTR, respectively, or comprising a reference 3′- and/or 5′-UTR, respectively, such as a 3′-UTR and/or 5′-UTR naturally occurring with the ORF of the artificial nucleic acid molecule.

Preferably, the present invention provides an artificial nucleic acid molecule comprising an ORF and a 3′-UTR element and/or a 5′-UTR element as described above, wherein the ratio of the protein amount, e.g. the amount of luciferase, observed 72 hours after initiation of expression to the protein amount observed 24 hours after initiation of expression, preferably in a mammalian expression system, such as in mammalian cells, e.g. in HeLa cells or HDF cells, is preferably above about 0.05, more preferably above about 0.1, more preferably above about 0.2, even more preferably above about 0.3, wherein preferably the total amount of protein produced from said artificial nucleic acid molecule, e.g. within a time span of 72 hours, is at least the total amount of protein produced, e.g. within said time span, from a reference nucleic acid molecule lacking a 3′- and/or 5′-UTR, respectively, or comprising a reference 3′- and/or 5′-UTR, respectively, such as a 3′- and/or 5′-UTR naturally occurring with the ORF of the artificial nucleic acid molecule.

“Increased protein expression” or “enhanced protein expression” in the context of the present invention preferably means an increased/enhanced protein expression at one point in time after initiation of expression or an increased/enhanced total amount of expressed protein compared to the expression induced by a reference nucleic acid molecule. Thus, the protein level observed at a certain point in time after initiation of expression, e.g. after transfection, of the artificial nucleic acid molecule according to the present invention, e.g. after transfection of an mRNA according to the present invention, for example, 6, 12, 24, 48 or 72 hours post transfection, is preferably higher than the protein level observed at the same point in time after initiation of expression, e.g. after transfection, of a reference nucleic acid molecule, such as a reference mRNA comprising a reference 3′- and/or 5′-UTR, respectively, or lacking a 3′- and/or 5′-UTR, respectively. In a preferred embodiment, the maximum amount of protein (as determined e.g. by protein activity or mass) expressed from the artificial nucleic acid molecule is increased with respect to the protein amount expressed from a reference nucleic acid comprising a reference 3′- and/or 5′-UTR, respectively, or lacking a 3′- and/or 5′-UTR, respectively. Peak expression levels are preferably reached within 48 hours, more preferably within 24 hours and even more preferably within 12 hours after, for instance, transfection.

Preferably, the term “increased total protein production” or “enhanced total protein production” from an artificial nucleic acid molecule according to the invention refers to an increased/enhanced protein production over a time span, in which protein is produced from an artificial nucleic acid molecule, e.g. 48 hours or 72 hours, preferably in a mammalian expression system, such as in mammalian cells, e.g. in HeLa or HDF cells in comparison to a reference nucleic acid molecule lacking a 3′- and/or 5′-UTR, respectively, or comprising a reference 3′- and/or 5′-UTR, respectively. According to a preferred embodiment, the cumulative amount of protein expressed over time is increased when using the artificial nucleic acid molecule according to the invention.

The total amount of protein for a specific time period may be determined by (i) collecting tissue or cells at several points in time after introduction of the artificial nucleic acid molecule (e.g. 6, 12, 24, 48 and 72 hours post initiation of expression or post introduction of the nucleic acid molecule), and the protein amount per point in time can be determined as explained above. In order to calculate the cumulative protein amount, a mathematical method of determining the total amount of protein can be used, e.g. the area under the curve (AUC) can be determined according to the following formula:

$$AUC = {\int }_{a}^{b} f ( x ) d ( x )$$

In order to calculate the area under the curve for total amount of protein, the integral of the equation of the expression curve from each end point (a and b) is calculated.

Thus, “total protein production” preferably refers to the area under the curve (AUC) representing protein production over time.

Preferably, the at least one 3′- or 5′-UTR element according to the present invention increases protein production from said artificial nucleic acid molecule at least 1.5 fold, preferably at least 2 fold, more preferably at least 2.5 fold, compared to the protein production from a reference nucleic acid molecule lacking a 3′- and/or 5′-UTR, respectively. In other words, the total amount of protein produced from in the artificial nucleic acid molecule according to the invention at a certain point in time, e.g. 48 hours or 72 hours post initiation of expression, e.g. post transfection, is increased by a factor of at least 1.5, preferably at least 2, more preferably at least 2.5, compared to the (relative) amount of protein produced from a reference nucleic acid molecule, which is e.g. lacking a 3′- and/or 5′-UTR, respectively, or comprising a reference 3′- and/or 5′-UTR, respectively, for the corresponding later point in time.

The mRNA and/or protein production prolonging effect and efficiency and/or the protein production increasing effect and efficiency of the variants, fragments and/or variant fragments of the 3′-UTR and/or the 5′-UTR as well as the mRNA and/or protein production prolonging effect and efficiency and/or the protein production increasing effect and efficiency of the at least one 3′-UTR element and/or the at least one 5′-UTR element of the artificial nucleic acid molecule according to the present invention may be determined by any method suitable for this purpose known to skilled person.

For example, artificial mRNA molecules may be generated comprising a coding sequence/open reading frame (ORF) for a reporter protein, such as luciferase, and a 3′-UTR element according to the present invention, i.e. which prolongs and/or increases protein production from said artificial mRNA molecule. In addition such an inventive mRNA molecule may further comprise a a 5′-UTR element according to the present invention, i.e. which prolongs and/or increases protein production from said artificial mRNA molecule, no 5′-UTR element or a 5′-UTR element which is not according to the present invention, e.g. a reference 5′-UTR. Accordingly, artificial mRNA molecules may be generated comprising a coding sequence/open reading frame (ORF) for a reporter protein, such as luciferase, and a 5′-UTR element according to the present invention, i.e. which prolongs and/or increases protein production from said artificial mRNA molecule. In addition such an inventive mRNA molecule may further comprise a a 3′-UTR element according to the present invention, i.e. which prolongs and/or increases protein production from said artificial mRNA molecule, no 3′-UTR element or a 3′-UTR element which is not according to the present invention, e.g. a reference 3′-UTR.

According to the present invention mRNAs may be generated, for example, by in vitro transcription of respective vectors such as plasmid vectors, e.g. comprising a T7 promoter and a sequence encoding the respective mRNA sequences. The generated mRNA molecules may be transfected into cells by any transfection method suitable for transfecting mRNA, for example they may be lipofected into mammalian cells, such as HeLa cells or HDF cells, and samples may be analyzed certain points in time after transfection, for example, 6 hours, 24 hours, 48 hours, and 72 hours post transfection. Said samples may be analyzed for mRNA quantities and/or protein quantities by methods well known to the skilled person. For example, the quantities of reporter mRNA present in the cells at the sample points in time may be determined by quantitative PCR methods. The quantities of reporter protein encoded by the respective mRNAs may be determined, e.g., by Western Blot, ELISA assays, FACS analysisor reporter assays such as luciferase assays depending on the reporter protein used. The effect of stabilizing protein expression and/or prolonging protein expression may be, for example, analyzed by determining the ratio of the protein level observed 48 hours post transfection and the protein level observed 24 hours post transfection. The closer said value is to 1, the more stable the protein expression is within this time period. Such measurements may of course also be performed at 72 or more hours and the ratio of the protein level observed 72 hours post transfection and the protein level observed 24 hours post transfection may be determined to determine stability of protein expression.

Moreover, the at least one 3′-UTR element and/or the at least one 5′-UTR element in the artificial nucleic acid molecule according to the present invention, is derived from a stable mRNA. Thereby, “derived” from a stable mRNA means that the at least one 3′-UTR element and/or the at least one 5′-UTR element shares at least 50%, preferably at least 60%, preferably at least 70%, more preferably at least 75%, more preferably at least 80%, more preferably at least 85%, even more preferably at least 90%, even more preferably at least 95%, and particularly preferably at least 98% sequence identity with a 3′-UTR element and/or a 5′-UTR element of a stable mRNA. Preferably, the stable mRNA is a naturally occurring mRNA and, thus, a 3′-UTR element and/or a 5′-UTR element of a stable mRNA refers to a 3′-UTR and/or a 5′-UTR, or fragments or variants thereof, of naturally occurring mRNA. Moreover, a 3′-UTR element and/or a 5′-UTR element derived from a stable mRNA preferably also refers to a 3′-UTR element and/or a 5′-UTR element, which is modified in comparison to a naturally occurring 3′-UTR element and/or 5′-UTR element, e.g. in order to increase RNA stability even further and/or to prolong and/or increase protein production. It goes without saying that such modifications are preferred, which do not impair RNA stability, e.g. in comparison to a naturally occurring (non-modified) 3′-UTR element and/or 5′-UTR element. In particular, the term mRNA as used herein refers to an mRNA molecule, however, it may also refer to an mRNA species as defined herein.

Preferably, the stability of mRNA, i.e. mRNA decay and/or half-life, is assessed under standard conditions, for example standard conditions (standard medium, incubation, etc.) for a certain cell line used.

The term “stable mRNA” as used herein refers in general to an mRNA having a slow mRNA decay. Thus, a “stable mRNA” has typically a long half-life. The half-life of an mRNA is the the time required for degrading 50% of the in vivo or in vitro existing mRNA molecules. Accordingly, stability of mRNA is usually assessed in vivo or in vitro. Thereby, in vitro refers in particular to (“living”) cells and/or tissue, including tissue of a living subject. Cells include in particular cell lines, primary cells, cells in tissue or subjects. In specific embodiments cell types allowing cell culture may be suitable for the present invention. Particularly preferred are mammalian cells, e.g. human cells and mouse cells. In particularly preferred embodiments the human cell lines HeLa, and U-937 and the mouse cell lines NIH3T3, JAWSII and L929 are used. Furthermore primary cells are particularly preferred, in particular preferred embodiments human dermal fibroblasts (HDF) may be used. Alternatively also a tissue of a subject may be used.

Preferably, the half-life of a “stable mRNA” is at least 5 h, at least 6 h, at least 7 h, at least 8 h, at least 9 h, at least 10 h, at least 11 h, at least 12 h, at least 13 h, at least 14 h, and/or at least 15 h. The half-life of an mRNA of interest may be determined by different methods known to the person skilled in the art. Typically, the half-life of an mRNA of interest is determined by determining the decay constant, whereby usually an ideal in vivo (or in vitro as defined above) situation is assumed, in which transcription of the mRNA of interest can be “turned off” completely (or at least to an undetectable level). In such an ideal situation it is usually assumed that mRNA decay follows first-order kinetics. Accordingly, the decay of an mRNA may usually be described by the following equation:

A(t)=A₀*e^{-λt}

with A₀ being the amount (or concentration) of the mRNA of interest at time 0, i.e. before the decay starts, A(t) being the amount (or concentration) of the mRNA of interest at a time t during decay and λ being the decay constant. Thus, if the amount (or concentration) of the mRNA of interest at time 0 (A₀) and the amount (or concentration) of the mRNA of interest at a certain time t during the decay process (A(t) and t) are known, the decay constant λ may be calculated. Based on the decay constant λ, the half-life t_{1/2} can be calculated by the following equation:

$${t}_{1 / 2} = ln 2 / λ .$$

since per definition A(t)/A0=1/2 at t_{1/2}. Thus, to assess the half-life of an mRNA of interest, usually the amount or concentration of the mRNA is determined during the RNA decay process in vivo (or in vitro as defined above).

To determine the amount or concentration of mRNA during the RNA decay process in vivo (or in vitro as defined above), various methods may be used, which are known to the skilled person. Non-limiting examples of such methods include general inhibition of transcription, e.g. with a transcription inhibitor such as actinomycin D, use of inducible promotors to specifically promote transient transcription, e.g. c-fos serum-inducible promotor system and Tet-off regulatory promotor system, and kinetic labelling techniques, e.g. pulse labelling, for example by 4-Thiouridine (4sU), 5-Ethynyluridine (EU) or 5′-Bromo-Uridine (BrU). Further details and preferred embodiments regarding how to determine the amount or concentration of mRNA during the RNA decay are outlined below, in the context of a method for identifying a 3′-UTR element and/or the at least one 5′-UTR element, which is derived from a stable mRNA, according to the present invention. The respective description and preferred embodiments of how to determine the amount or concentration of mRNA during the RNA decay apply here as well.

Preferably, a “stable mRNA” in the sense of the present invention has a slower mRNA decay compared to average mRNA, preferably assessed in vivo (or in vitro as defined above). For example, “average mRNA decay” may be assessed by investigating mRNA decay of a plurality of mRNA species, preferably 100, at least 300, at least 500, at least 1000, at least 2000, at least 3000, at least 4000, at least 5000, at least 6000, at least 7000, at least 8000, at least 9000, at least 10000, at least 11000, at least 12000, at least 13000, at least 14000, at least 15000, at least 16000, at least 17000, at least 18000, at least 19000, at least 20000, at least 21000, at least 22000, at least 23000, at least 24000, at least 25000, at least 26000, at least 27000, at least 28000, at least 29000, at least 30000 mRNA species. It is particularly preferred that the whole transcriptome is assessed, or as many mRNA species of the transcriptome as possible. This may be achieved, for example, by using a micro array providing whole transcript coverage.

An “mRNA species”, as used herein, corresponds to a genomic transcription unit, i.e. usually to a gene. Thus, within one “mRNA species” different transcripts may occur, for example, due to mRNA processing. For example, an mRNA species may be represented by a spot on a microarray. Accordingly, a microarray provides an advantageous tool to determine the amount of a plurality of mRNA species, e.g. at a certain point in time during mRNA decay. However, also other techniques known to the skilled person, e.g. RNA-seq, quantitative PCR etc. may be used.

In the present invention it is particularly preferred that a stable mRNA is characterized by an mRNA decay wherein the ratio of the amount of said mRNA at a second point in time to the amount of said mRNA at a first point in time is at least 0.5 (50%), at least 0.6 (60%), at least 0.7 (70%), at least 0.75 (75%), at least 0.8 (80%), at least 0.85 (85%), at least 0.9 (90%), or at least 0.95 (95%). Thereby, the second point in time is later in the decay process than the first point in time.

Preferably, the first point in time is selected such that only mRNA undergoing a decay process is considered, i.e. emerging mRNA—e.g. in ongoing transcription—is avoided. For example, if kinetic labelling techniques, e.g. pulse labelling, are used, the first point in time is preferably selected such that the incorporation of the label into mRNA is completed, i.e. no ongoing incorporation of the label into mRNA occurs. Thus, if kinetic labelling is used, the first point in time may be at least 10 min, at least 20 min, at least 30 min, at least 40 min, at least 50 min, at least 60 min, at least 70 min, at least 80 min, or at least 90 min after the end of the experimental labelling procedure, e.g. after the end of the incubation of cells with the label.

For example, the first point in time may be preferably from 0 to 6 h after the stop of transcription (e.g. by a transcriptional inhibitor), stop of promotor induction in case of inducible promotors or after stop of pulse or label supply, e.g. after end of labelling. More preferably, the first point in time may be 30 min to 5 h, even more preferably 1 h to 4 h and particularly preferably about 3 h after the stop of transcription (e.g. by a transcriptional inhibitor), stop of promotor induction in case of inducible promotors or after stop of pulse or label supply, e.g. after end of labelling.

Preferably, the second point in time is selected as late as possible during the mRNA decay process. However, if a plurality of mRNA species is considered, the second point in time is preferably selected such that still a considerable amount of the plurality of mRNA species, preferably at least 10% of the mRNA species, is present in a detectable amount, i.e. in an amount higher than 0. Preferably, the second point in time is at least 5 h, at least 6 h, at least 7 h, at least 8 h, at least 9 h, at least 10 h, at least 11 h, at least 12 h, at least 13 h, at least 14 h, or at least 15 h after the end of transcription or the end of the experimental labelling procedure.

Thus, the time span between the first point in time and the second point in time is preferably as large as possible within the above described limits. Therefore, the time span between the first point in time and the second point in time is preferably at least 4 h, at least 5 h, at least 6 h, at least 7 h, at least 8 h, at least 9 h, at least 10 h, at least 11 h, or at least 12 h.

Moreover, it is preferred that the at least one 3′-UTR element and/or the at least one 5′-UTR element in the artificial nucleic acid molecule according to the present invention, which is derived from a stable mRNA, is identified by a method for identifying a 3′-UTR element and/or a 5′-UTR element, which is derived from a stable mRNA, according to the present invention as described herein. It is particularly preferred that the at least one 3′-UTR element and/or the at least one 5′-UTR element in the artificial nucleic acid molecule according to the present invention, is identified by a method for identifying a 3′-UTR element and/or a 5′-UTR element, which prolongs and/or increases protein production from an artificial nucleic acid molecule and which is derived from a stable mRNA, according to the present invention as described herein.

Preferably, the at least one 3′-UTR element and/or the at least one 5′-UTR element in the artificial nucleic acid molecule according to the present invention comprises or consists of a nucleic acid sequence which is derived from the 3′-UTR and/or the 5′-UTR of a eukaryotic protein coding gene, preferably from the 3′-UTR and/or the 5′-UTR of a vertebrate protein coding gene, more preferably from the 3′-UTR and/or the 5′-UTR of a mammalian protein coding gene, e.g. from mouse and human protein coding genes, even more preferably from the 3′-UTR and/or the 5′-UTR of a primate or rodent protein coding gene, in particular the 3′-UTR and/or the 5′-UTR of a human or murine protein coding gene.

In general, it is understood that the at least one 3′-UTR element in the artificial nucleic acid molecule according to the present invention comprises or consists of a nucleic acid sequence which is preferably derived from a naturally (in nature) occurring 3′-UTR, whereas the at least one 5′-UTR element in the artificial nucleic acid molecule according to the present invention comprises or consists of a nucleic acid sequence which is preferably derived from a naturally (in nature) occurring 5′-UTR.

Preferably, the at least one open reading frame is heterologous to the at least one 3′-UTR element and/or to the at least one 5′-UTR element. The term “heterologous” in this context means that two sequence elements comprised by the artificial nucleic acid molecule, such as the open reading frame and the 3′-UTR element and/or the open reading frame and the 5′-UTR element, do not occur naturally (in nature) in this combination. They are typically recombinant. Preferably, the 3′-UTR element and/or the 5′-UTR element are/is derived from a different gene than the open reading frame. For example, the ORF may be derived from a different gene than the 3′-UTR element and/or to the at least one 5′-UTR element, e.g. encoding a different protein or the same protein but of a different species etc. I.e. the open reading frame is derived from a gene which is distinct from the gene from which the 3′-UTR element and/or to the at least one 5′-UTR element is derived. In a preferred embodiment, the ORF does not encode a human or plant (e.g., Arabidopsis) ribosomal protein, preferably does not encode human ribosomal protein S6 (RPS6), human ribosomal protein L36a-like (RPL36AL) or Arabidopsis ribosomal protein S16 (RPS16). In a further preferred embodiment, the open reading frame (ORF) does not encode ribosomal protein S6 (RPS6), ribosomal protein L36a-like (RPL36AL) or ribosomal protein S16 (RPS16).

In specific embodiments it is preferred that the open reading frame does not code for a reporter protein, e.g., selected from the group consisting of globin proteins (particularly beta-globin), luciferase protein, GFP proteins or variants thereof, for example, variants exhibiting at least 70% sequence identity to a globin protein, a luciferase protein, or a GFP protein. Thereby, it is particularly preferred that the open reading frame does not code for a GFP protein. It is also particularly preferred that the open reading frame (ORF) does not encode a reporter gene or is not derived from a reporter gene, wherein the reporter gene is preferably not selected from group consisting of globin proteins (particularly beta-globin), luciferase protein, beta-glucuronidase (GUS) and GFP proteins or variants thereof, preferably not selected from EGFP, or variants of any of the above genes, typically exhibiting at least 70% sequence identity to any of these reporter genes, preferably a globin protein, a luciferase protein, or a GFP protein.

Even more preferably, the 3′-UTR element and/or the 5′-UTR element is heterologous to any other element comprised in the artificial nucleic acid as defined herein. For example, if the artificial nucleic acid according to the invention comprises a 3′-UTR element from a given gene, it does preferably not comprise any other nucleic acid sequence, in particular no functional nucleic acid sequence (e.g. coding or regulatory sequence element) from the same gene, including its regulatory sequences at the 5′ and 3′ terminus of the gene's ORF. Accordingly, for example, if the artificial nucleic acid according to the invention comprises a 5′-UTR element from a given gene, it does preferably not comprise any other nucleic acid sequence, in particular no functional nucleic acid sequence (e.g. coding or regulatory sequence element) from the same gene, including its regulatory sequences at the 5′ and 3′ terminus of the gene's ORF.

Moreover, it is preferred that the artificial nucleic acid according to the present invention comprises at least one open reading frame, at least one 3′-UTR (element) and at least one 5′-UTR (element), whereby either the at least one 3′-UTR (element) is a 3′-UTR element according to the present invention and/or the at least one 5′-UTR (element) is a 5′-UTR element according to the present invention. In such a preferred artificial nucleic acid according to the present invention, which comprises at least one open reading frame, at least one 3′-UTR (element) and at least one 5′-UTR (element), it is particularly preferred that each of the at least one open reading frame, the at least one 3′-UTR (element) and the at least one 5′-UTR (element) are heterologous, i.e. neither the at least one 3′-UTR (element) and the at least one 5′-UTR (element) nor the the open reading frame and the 3′-UTR (element) or the 5′-UTR (element), respectively, are occurring naturally (in nature) in this combination. This means that the artificial nucleic acid molecule comprises an ORF, a 3′-UTR (element) and a 5′-UTR (element), all of which are heterologous to each other, e.g. they are recombinant as each of them is derived from different genes (and their 5′ and 3′ UTR's). In another preferred embodiment, the 3′-UTR (element) is not derived from a 3′-UTR (element) of a viral gene or is not of viral origin.

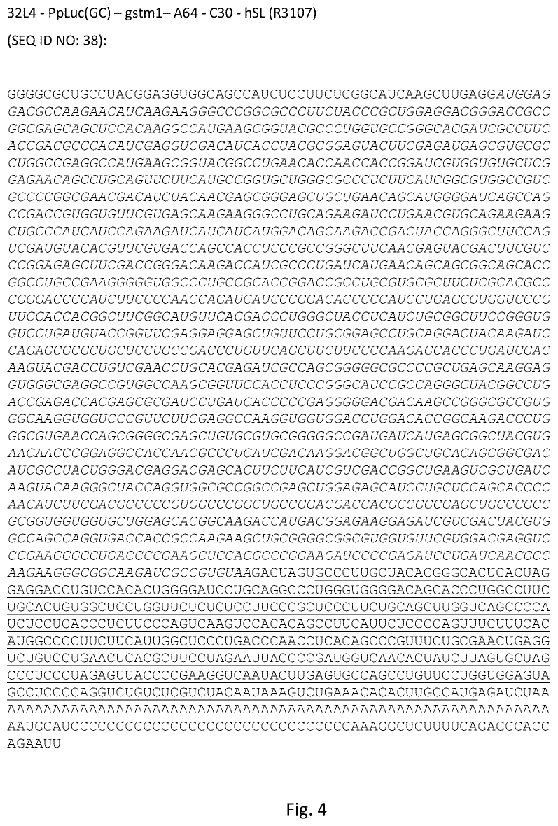

Preferably, the artificial nucleic acid molecule according to the present invention:
(i) comprises at least one 3′-UTR element and at least one 5′-UTR element, wherein preferably (each of) the at least one 3′-UTR element and at least one 5′-UTR element comprises or consists of a nucleic acid sequence which is derived from the 3′-UTR, or the 5′-UTR respectively, of a human or murine protein coding gene;
(ii) the at least one 3′-UTR element, the at least one 5′-UTR element and the at least one open reading frame of the artificial nucleic acid molecule according to the present invention are all heterologous to each other;
(iii) the at least one 3′ UTR element is derived from a gene selected from the group consisting of: housekeeping genes, genes coding for a membrane protein, genes involved in cellular metabolism, genes involved in transcription, translation and replication processes, genes involved in protein modification and genes involved in cell division; and
(iv) the 3′UTR is not derived from a gene coding for a ribosomal protein or from the FIG. 4 gene.

Housekeeping genes are typically constitutive genes that are required for the maintenance of basic cellular function and that are typically expressed in all cells of an organism under normal and patho-physiological conditions. Although some housekeeping genes are expressed at relatively constant levels in most non-pathological situations, other housekeeping genes may vary depending on experimental conditions. Typically, housekeeping genes are expressed in at least 25 copies per cell and sometimes number in the thousands. Preferred examples of housekeeping genes in the context of the present invention are shown below in Table 10.

TABLE 10
List of abundant housekeeping genes (cf. WO 2007/068265 A1, Table 1).

| Acc | Definition | Symbol^{a} | Length^{b} | Abundance^{c} |
|---|---|---|---|---|
| NM_001402 | Eukaryotic translation elongation factor 1 alpha 1 | EEF1A1 | 387 | 20011 |
| NM_001614 | Actin, gamma 1 | ACTG1 | 718 | 16084 |
| NM_002046 | Glyceraldehyde-3-phosphate dehydrogenase | GAPD | 201 | 15931 |
| NM_001101 | Actin, beta | ACTB | 593 | 15733 |
| NM_000967 | Ribosomal protein L3 | RPL3 | 74 | 10924 |
| NM_006082 | Tubulin, alpha, ubiquitous | K-ALPHA-1 | 174 | 10416 |
| NM_001428 | Enolase 1, (alpha) | ENO1 | 357 | 9816 |
| NM_006098 | Guanine nucleotide binding protein (G protein), beta polypeptide 2- | GNB2L1 | 45 | 8910 |
| | like 1 | | | |
| NM_002032 | Ferritin, heavy polypeptide 1 | FTH1 | 138 | 8861 |
| NM_002654 | Pyruvate kinase, muscle | PKM2 | 643 | 7413 |
| NM_004048 | Beta-2-microglobulin | B2M | 568 | 7142 |
| NM_006597 | Heat shock 70 kDa protein 8 | HSPA8 | 258 | 6066 |
| NM_000034 | Aldolase A, fructose-bisphosphate | ALDOA | 252 | 5703 |
| NM_021009 | Ubiquitin C | UBC | 67 | 5579 |
| NM_006013 | Ribosomal protein L10 | RPL10 | 1,503 | 5572 |
| NM_012423 | Ribosomal protein L13a | RPL13A | 509 | 5552 |
| NM_007355 | Heat shock 90 kDa protein 1, beta | HSPCB | 309 | 5436 |
| NM_004046 | ATP synthase, H+ transporting, mitochondrial F1 complex, alpha | ATP5A1 | 164 | 5434 |
| | subunit, isoform 1, cardiac muscle | | | |
| NM_000516 | GNAS complex locus | GNAS | 362 | 4677 |
| NM_001743 | Calmodulin 2 (phosphorylase kinase, delta) | CALM2 | 611 | 4306 |
| NM_005566 | Lactate dehydrogenase A | LDHA | 566 | 4186 |
| NM_000973 | Ribosomal protein L8 | RPL8 | 92 | 4042 |
| NM_002948 | Ribosomal protein L15 | RPL15 | 1,368 | 3861 |
| NM_000977 | Ribosomal protein L13 | RPL13 | 424 | 3774 |
| NM_002952 | Ribosomal protein S2 | RPS2 | 86 | 3758 |
| NM_005507 | Cofilin 1 (non-muscle) | CFL1 | 508 | 3616 |
| NM_004039 | Annexin A2 | ANXA2 | 294 | 3560 |
| NM_021019 | Myosin, light polypeptide 6, alkali, smooth muscle and non-muscle | MYL6 | 209 | 3512 |
| NM_002300 | Lactate dehydrogenase B | LDHB | 230 | 3501 |
| NM_003217 | Testis enhanced gene transcript (BAX inhibitor 1) | TEGT | 1,847 | 3438 |
| NM_002568 | Poly(A) binding protein, cytoplasmic 1 | PABPC1 | 445 | 3241 |
| NM_001015 | Ribosomal protein S11 | RPS11 | 85 | 3220 |
| NM_003973 | Ribosomal protein L14 | RPL14 | 156 | 3198 |
| NM_000969 | Ribosomal protein L5 | RPL5 | 78 | 3167 |
| NM_007104 | Ribosomal protein L10a | RPL10A | 32 | 3079 |
| NM_001642 | Amyloid beta (A4) precursor-like protein 2 | APLP2 | 1,364 | 3002 |
| NM_001418 | Eukaryotic translation initiation factor 4 gamma, 2 | EIF4G2 | 791 | 2913 |
| NM_002635 | Solute carrier family 25 (mitochondrial carrier; phosphate carrier), | SLC25A3 | 197 | 2900 |
| | member 3 | | | |
| NM_001009 | Ribosomal protein S5 | RPS5 | 58 | 2897 |
| NM_000291 | Phosphoglycerate kinase 1 | PGK1 | 1,016 | 2858 |
| NM_001728 | Basigin (OK blood group) | BSG | 769 | 2827 |
| NM_001658 | ADP-ribosylation factor 1 | ARF1 | 1,194 | 2772 |
| NM_001003 | Ribosomal protein, large, P1 | RPLP1 | 39 | 2770 |
| NM_018955 | Ubiquitin B | UBB | 144 | 2732 |
| NM_005998 | Chaperonin containing TCP1, subunit 3 (gamma) | CCT3 | 255 | 2709 |
| NM_001967 | Eukaryotic translation initiation factor 4A, isoform 2 | EIF4A2 | 626 | 2693 |
| NM_001469 | Thyroid autoantigen 70 kDa (Ku antigen) | G22P1 | 259 | 2682 |
| NM_000918 | Procollagen-proline, 2-oxoglutarate 4-dioxygenase (proline 4- | P4HB | 868 | 2659 |
| | hydroxylase), beta polypeptide (protein disulfide isomerase; thyroid | | | |
| | hormone binding protein p55) | | | |
| NM_002574 | Peroxiredoxin 1 | PRDX1 | 323 | 2604 |
| NM_001020 | Ribosomal protein S16 | RPS16 | 78 | 2573 |
| NM_007363 | Non-POU domain containing, octamer-binding | NONO | 1,119 | 2557 |
| NM_001022 | Ribosomal protein S19 | RPS19 | 63 | 2533 |
| NM_001675 | Activating transcription factor 4 (tax-responsive enhancer element | ATF4 | 85 | 2479 |
| | B67) | | | |
| NM_005617 | Ribosomal protein S14 | RPS14 | 78 | 2465 |
| NM_001664 | Ras homolog gene family, member A | RHOA | 1,045 | 2426 |
| NM_005801 | Putative translation initiation factor | SUI1 | 836 | 2425 |
| NM_000981 | Ribosomal protein L19 | RPL19 | 80 | 2381 |
| NM_000979 | Ribosomal protein L18 | RPL18 | 49 | 2362 |
| NM_001026 | Ribosomal protein S24 | RPS24 | 77 | 2355 |
| NM_000975 | Ribosomal protein L11 | RPL11 | 53 | 2314 |
| NM_002117 | Major histocompatibility complex, class I, C | HLA-C | 434 | 2278 |
| NM_004068 | Adaptor-related protein complex 2, mu 1 subunit | AP2M1 | 494 | 2230 |
| NM_006429 | Chaperonin containing TCP1, subunit 7 (eta) | CCT7 | 164 | 2216 |
| NM_022551 | Ribosomal protein S18 | RPS18 | 5,538 | 2208 |
| NM_001013 | Ribosomal protein S9 | RPS9 | 73 | 2113 |
| NM_005594 | Nascent-polypeptide-associated complex alpha polypeptide | NACA | 133 | 2075 |
| NM_001028 | Ribosomal protein S25 | RPS25 | 74 | 2066 |
| NM_032378 | Eukaryotic translation elongation factor 1 delta (guanine nucleotide | EEF1D | 76 | 2051 |
| | exchange protein) | | | |
| NM_000999 | Ribosomal protein L38 | RPL38 | 50 | 2007 |
| NM_000994 | Ribosomal protein L32 | RPL32 | 64 | 2003 |
| NM_007008 | Reticulon 4 | RTN4 | 973 | 1969 |
| NM_001909 | Cathepsin D (lysosomal aspartyl protease) | CTSD | 834 | 1940 |
| NM_006325 | RAN, member RAS oncogene family | RAN | 892 | 1906 |
| NM_003406 | Tyrosine 3-monooxygenase/tryptophan 5-monooxygenase | YWHAZ | 2,013 | 1892 |
| | activation protein, zeta polypeptide | | | |
| NM_006888 | Calmodulin 1 (phosphorylase kinase, delta) | CALM1 | 3,067 | 1880 |
| NM_004339 | Pituitary tumor-transforming 1 interacting protein | PTTG1IP | 1,985 | 1837 |
| NM_005022 | Profilin 1 | PFN1 | 289 | 1787 |
| NM_001961 | Eukaryotic translation elongation factor 2 | EEF2 | 504 | 1754 |
| NM_003091 | Small nuclear ribonucleoprotein polypeptides B and B1 | SNRPB | 295 | 1735 |
| NM_006826 | Tyrosine 3-monooxygenase/tryptophan 5-monooxygenase | YWHAQ | 1,310 | 1726 |
| | activation protein, theta polypeptide | | | |
| NM_002140 | Heterogeneous nuclear ribonucleoprotein K | HNRPK | 1,227 | 1725 |
| NM_001064 | Transketolase (Wernicke-Korsakoff syndrome) | TKT | 167 | 1721 |
| NM_021103 | Thymosin, beta 10 | TMSB10 | 317 | 1714 |
| NM_004309 | Rho GDP dissociation inhibitor (GDI) alpha | ARHGDIA | 1,206 | 1702 |
| NM_002473 | Myosin, heavy polypeptide 9, non-muscle | MYH9 | 1,392 | 1692 |
| NM_000884 | IMP (inosine monophosphate) dehydrogenase 2 | IMPDH2 | 63 | 1690 |
| NM_001004 | Ribosomal protein, large P2 | RPLP2 | 59 | 1688 |
| NM_001746 | Calnexin | CANX | 2,302 | 1677 |
| NM_002819 | Polypyrimidine tract binding protein 1 | PTBP1 | 1,561 | 1663 |
| NM_000988 | Ribosomal protein L27 | RPL27 | 59 | 1660 |
| NM_004404 | Neural precursor cell expressed, developmentally down-regulated 5 | NEDD5 | 2,090 | 1654 |
| NM_005347 | Heat shock 70 kDa protein 5 (glucose-regulated protein, 78 kDa) | HSPA5 | 1,757 | 1651 |
| NM_000175 | Glucose phosphate isomerase | GPI | 296 | 1635 |
| NM_001207 | Basic transcription factor 3 | BTF3 | 300 | 1632 |
| NM_003186 | Transgelin | TAGLN | 405 | 1612 |
| NM_003334 | Ubiquitin-activating enzyme E1 (A1S9T and BN75 temperature | UBE1 | 199 | 1590 |
| | sensitivity complementing) | | | |
| NM_001018 | Ribosomal protein S15 | RPS15 | 32 | 1574 |
| NM_003404 | Tyrosine 3-monooxygenase/tryptophan 5-monooxygenase | YWHAB | 2,088 | 1523 |
| | activation protein, beta polypeptide | | | |
| NM_003753 | Eukaryotic translation initiation factor 3, subunit 7 zeta, 66/67 kDa | EIF3S7 | 152 | 1509 |
| NM_005762 | Tripartite motif-containing 28 | TRIM28 | 193 | 1507 |
| NM_005381 | Nucleolin | NCL | 284 | 1501 |
| NM_000995 | Ribosomal protein L34 | RPL34 | 450 | 1495 |
| NM_002823 | Prothymosin, alpha (gene sequence 28) | PTMA | 720 | 1462 |
| NM_002415 | Macrophage migration inhibitory factor (glycosylation-inhibiting | MIF | 117 | 1459 |
| | factor) | | | |
| NM_002128 | High-mobility group box 1 | HMGB1 | 1,527 | 1457 |
| NM_006908 | Ras-related C3 botulinum toxin substrate 1 (rho family, small GTP | RAC1 | 1,536 | 1437 |
| | binding protein Rac1) | | | |
| NM_002070 | Guanine nucleotide binding protein (G protein), alpha inhibiting | GNAI2 | 512 | 1435 |
| | activity polypeptide 2 | | | |
| NM_001997 | Finkel-Biskis-Reilly murine sarcoma virus (FBR-MuSV) ubiquitously | FAU | 68 | 1428 |
| | expressed (fox derived); ribosomal protein S30 | | | |
| NM_014390 | Staphylococcal nuclease domain containing 1 | SND1 | 556 | 1422 |
| NM_014764 | DAZ associated protein 2 | DAZAP2 | 1,322 | 1419 |
| NM_005917 | Malate dehydrogenase 1, NAD (soluble) | MDH1 | 208 | 1396 |
| NM_001494 | GDP dissociation inhibitor 2 | GDI2 | 785 | 1395 |
| NM_014225 | Protein phosphatase 2 (formerly 2A), regulatory subunit A (PR 65), | PPP2R1A | 472 | 1391 |
| | alpha isoform | | | |
| NM_001660 | ADP-ribosylation factor 4 | ARF4 | 858 | 1382 |
| NM_001823 | Creatine kinase, brain | CKB | 206 | 1381 |
| NM_003379 | Villin 2 (ezrin) | VIL2 | 1,272 | 1380 |
| NM_000182 | Hydroxyacyl-Coenzyme A dehydrogenase/3-ketoacyl-Coenzyme A | HADHA | 647 | 1379 |
| | thiolase/enoyl-Coenzyme A hydratase (trifunctional protein), alpha | | | |
| | subunit | | | |
| NM_003746 | Dynein, cytoplasmic, light polypeptide 1 | DNCL1 | 281 | 1375 |
| NM_007103 | NADH dehydrogenase (ubiquinone) flavoprotein 1, 51 kDa | NDUFV1 | 103 | 1352 |
| NM_000992 | Ribosomal protein L29 | RPL29 | 164 | 1349 |
| NM_007209 | Ribosomal protein L35 | RPL35 | 35 | 1345 |
| NM_006623 | Phosphoglycerate dehydrogenase | PHGDH | 231 | 1340 |
| NM_002796 | Proteasome (prosome, macropain) subunit, beta type, 4 | PSMB4 | 108 | 1340 |
| NM_002808 | Proteasome (prosome, macropain) 26S subunit, non-ATPase, 2 | PSMD2 | 231 | 1326 |
| NM_000454 | Superoxide dismutase 1, soluble (amyotrophic lateral sclerosis 1 | SOD1 | 346 | 1323 |
| | (adult)) | | | |
| NM_003915 | RNA binding motif protein 12 | RBM12 | 216 | 1323 |
| NM_004924 | Actinin, alpha 4 | ACTN4 | 1,099 | 1316 |
| NM_006086 | Tubulin, beta 3 | TUBB3 | 296 | 1314 |
| NM_001016 | Ribosomal protein S12 | RPS12 | 56 | 1304 |
| NM_003365 | Ubiquinol-cytochrome c reductase core protein I | UQCRC1 | 126 | 1303 |
| NM_003016 | Splicing factor, arginine/serine-rich 2 | SFRS2 | 1,059 | 1301 |
| NM_007273 | Repressor of estrogen receptor activity | REA | 332 | 1281 |
| NM_014610 | Glucosidase, alpha; neutral AB | GANAB | 1,652 | 1280 |
| NM_001749 | Calpain, small subunit 1 | CAPNS1 | 514 | 1270 |
| NM_005080 | X-box binding protein 1 | XBP1 | 1,003 | 1269 |
| NM_005216 | Dolichyl-diphosphooligosaccharide-protein glycosyltransferase | DDOST | 616 | 1268 |
| NM_004640 | HLA-B associated transcript 1 | BAT1 | 237 | 1262 |
| NM_021983 | Major histocompatibility complex, class II, DR beta 4 | HLA- | 313 | 1251 |
| | | DRB1 | | |
| NM_013234 | Eukaryotic translation initiation factor 3 subunit k | eIF3k | 84 | 1251 |
| NM_004515 | Interleukin enhancer binding factor 2, 45 kDa | ILF2 | 384 | 1249 |
| NM_000997 | Ribosomal protein L37 | RPL37 | 50 | 1244 |
| NM_000801 | FK506 binding protein 1A, 12 kDa | FKBP1A | 1,149 | 1243 |
| NM_000985 | Ribosomal protein L17 | RPL17 | 58 | 1243 |
| NM_001014 | Ribosomal protein S10 | RPS10 | 57 | 1232 |
| NM_001069 | Tubulin, beta 2 | TUBB2 | 194 | 1230 |
| NM_004960 | Fusion (involved in t(12; 16) in malignant liposarcoma) | FUS | 166 | 1197 |
| NM_005165 | Aldolase C, fructose-bisphosphate | ALDOC | 432 | 1195 |
| NM_004930 | Capping protein (actin filament) muscle Z-line, beta | CAPZB | 259 | 1193 |
| NM_000239 | Lysozyme (renal amyloidosis) | LYZ | 1,016 | 1190 |
| NM_007263 | Coatomer protein complex, subunit epsilon | COPE | 263 | 1179 |
| NM_001861 | Cytochrome c oxidase subunit IV isoform 1 | COX4I1 | 129 | 1178 |
| NM_003757 | Eukaryotic translation initiation factor 3, subunit 2 beta, 36 kDa | EIF3S2 | 408 | 1169 |
| NM_005745 | B-cell receptor-associated protein 31 | BCAP31 | 438 | 1166 |
| NM_002743 | Protein kinase C substrate 80K-H | PRKCSH | 337 | 1158 |
| NM_004161 | RAB1A, member RAS oncogene family | RAB1A | 638 | 1115 |
| NM_002080 | Glutamic-oxaloacetic transaminase 2, mitochondrial (aspartate | GOT2 | 1,039 | 1114 |
| | aminotransferase 2) | | | |
| NM_005731 | Actin related protein 2/3 complex, subunit 2, 34 kDa | ARPC2 | 448 | 1113 |
| NM_006445 | PRP8 pre-mRNA processing factor 8 homolog (yeast) | PRPF8 | 173 | 1110 |
| NM_001867 | Cytochrome c oxidase subunit VIIc | COX7C | 168 | 1106 |
| NM_002375 | Microtubule-associated protein 4 | MAP4 | 1,164 | 1102 |
| NM_003145 | Signal sequence receptor, beta (translocon-associated protein beta) | SSR2 | 492 | 1099 |
| NM_001788 | CDC10 cell division cycle 10 homolog (S. cerevisiae) | CDC10 | 1,015 | 1094 |
| NM_006513 | Seryl-tRNA synthetase | SARS | 323 | 1085 |
| NM_003754 | Eukaryotic translation initiation factor 3, subunit 5 epsilon, 47 kDa | EIF3S5 | 152 | 1081 |
| NM_005112 | WD repeat domain 1 | WDR1 | 845 | 1080 |
| NM_004893 | H2A histone family, member Y | H2AFY | 635 | 1072 |
| NM_004494 | Hepatoma-derived growth factor (high-mobility group protein 1-like) | HDGF | 1,339 | 1069 |
| NM_001436 | Fibrillarin | FBL | 111 | 1069 |
| NM_003752 | Eukaryotic translation initiation factor 3, subunit 8, 110 kDa | EIF3S8 | 201 | 1060 |
| NM_003321 | Tu translation elongation factor, mitochondrial | TUFM | 207 | 1038 |
| NM_001119 | Adducin 1 (alpha) | ADD1 | 1,569 | 1037 |
| NM_005273 | Guanine nucleotide binding protein (G protein), beta polypeptide 2 | GNB2 | 386 | 1030 |
| NM_006755 | Transaldolase 1 | TALDO1 | 256 | 1026 |
| NM_023009 | MARCKS-like 1 | MARCKSL1 | 774 | 1014 |
| NM_002799 | Proteasome (prosome, macropain) subunit, beta type, 7 | PSMB7 | 162 | 1012 |
| NM_002539 | Ornithine decarboxylase 1 | ODC1 | 343 | 1009 |
| NM_006801 | KDEL (Lys-Asp-Glu-Leu) endoplasmic reticulum protein retention | KDELR1 | 742 | 1007 |
| | receptor 1 | | | |
| NM_014944 | Calsyntenin 1 | CLSTN1 | 1,481 | 1003 |
| NM_007262 | Parkinson disease (autosomal recessive, early onset) 7 | PARK7 | 253 | 1002 |

The above table was obtained from WO 2007/068265 A1, Table 1 and is based on the list of the accession numbers as provided by Eisenberg, E. and E. Y. Levanon (2003): Human housekeeping genes are compact; Trends Genet. 19(7): 362-365. The accession numbers were used as input for a PERL (Programmed Extraction Report Language) computer program that extracts EST data from the Unigene database. The Unigene database was downloaded as a text file from the NCBI website. The length of the 3′UTR was derived by computationally extracting the 3′UTR (Bakheet, T., Frevel, M., Williams, BR, and K. S. Khabar, 2001. ARED: Human AU-rich element-containing mRNA database reveals unexpectedly diverse functional repertoire of encoded proteins. Nucleic Acids Research. 29:246-254). <a> is a commonly used abbreviation of the gene product; <b> is the length of the 3′UTR; <c> is the number of ESTs.

Preferred housekeeping genes include LDHA, NONO, PGK1 and PPIH.

A gene coding for a membrane protein typically refers to such a gene, which codes for a protein that interacts with biological membranes. In most genomes, about 20-30% of all genes encode membrane proteins. Common types of proteins include—in addition to membrane proteins—soluble globular proteins, fibrous proteins and disordered proteins. Thus, genes coding for a membrane protein are typically different from genes coding for soluble globular proteins, fibrous proteins or disordered proteins. Membrane proteins include membrane receptors, transport proteins, membrane enzymes and cell adhesion molecules.

A gene involved in cellular metabolism typically refers to such a gene, which codes for a protein involved in cellular metabolism, i.e. in the set of life-sustaining chemical transformations within the cells of living organisms. These are typically enzyme-catalyzed reactions, which allow organisms to grow and reproduce, maintain their structures, and respond to their environments. Accordingly, preferred genes involved in cellular metabolism are such genes, which code for enzymes catalyzing a reaction, which allow organisms to grow and reproduce, maintain their structures, and respond to their environments. Other examples for a gene involved in cellular metabolism include genes coding for proteins having structural or mechanical function, such as those that form the cytoskeleton. Other proteins involved in cellular metabolism include proteins involved in cell signalling, immune responses, cell adhesion, active transport across membranes and in the cell cycle. Metabolism is usually divided into two categories: catabolism, the breaking down of organic matter by way of cellular respiration, and anabolism, the building up of components of cells such as proteins and nucleic acids.

A gene involved in transcription, translation and replication processes typically refers to such a gene, which codes for a protein involved in transcription, translation and replication processes. In particular, the term “replication”, as used in this context, refers preferably to replication of nucleic acids, e.g. DNA replication. Preferred genes involved in transcription, translation and replication processes are genes coding for an enzyme involved in transcription, translation and/or (DNA) replication processes. Other preferred examples include genes coding for a transcription factor or for a translation factor. Ribosomal genes are other preferred examples of genes involved in transcription, translation and replication processes.

A gene involved in protein modification typically refers to such a gene, which codes for a protein involved in protein modification. Preferred examples of such genes code for enzymes involved in protein modification, in particular in post-translational modification processes. Preferred examples of enzymes involved in post-translational modification include (i) enzymes involved in the addition of hydrophobic groups, in particular for membrane localization, e.g. enzymes involed in myristoylation, palmitoylation, isoprenylation or prenylation, farnesylation, geranylation or in glypiation; (ii) enzymes involved in the addition of cofactors for enhanced enzymatic activity, e.g. enzymes involved in lipoylation, in the attachment of a flavin moiety, in the attachment of heme C, in phosphopantetheinylation or in retinylidene Schiff base formation; (iii) enzymes involved in the modification of translation factors, e.g. in diphtamide formation, in ethanolamine phosphoglycerol attachment or in hypusine formation; and (vi) enzymes involved in the addition of smaller chemical groups, e.g. acylation, such as acetylation and formylation, alkylation such as methylation, amide bond formation, such as amidation at C-terminus and amino acid addition (e.g. arginylation, polyglutamylation and polyglycylation), butyrylation, gamma-carboxylation, glycosylation, malonylation, hydroxylation, iodination, nucleotide addition, oxidation, phosphate ester or phosphoramidate formation such as phosphorylation and adenylation, propionylation, pyroglutamate formation, S-glutathionylation, S-nitrosylation, succinylation and sulfation.

A gene involved in cell division processes typically refers to such a gene, which codes for a protein involved in cell division. Cell division is the process by which a parent cell divides into two or more daughter cells. Cell division usually occurs as part of a larger cell cycle. In eukaryotes, there are two distinct types of cell division: a vegetative division, whereby each daughter cell is genetically identical to the parent cell (mitosis), and a reductive cell division, whereby the number of chromosomes in the daughter cells is reduced by half, to produce haploid gametes (meiosis). Accordingly, preferred gene involved in cell division processes code for a protein involved in mitosis and/or meiosis.

FIG. 4 is an abbreviation for Factor-Induced Gene. The FIG. 4 gene codes for polyphosphoinositide phosphatase also known as phosphatidylinositol 3,5-bisphosphate 5-phosphatase or SAC domain-containing protein 3 (Sac3).

Preferably, the artificial nucleic acid molecule according to the present invention:
(i) comprises at least one 3′-UTR element and at least one 5′-UTR element, wherein preferably (each of) the at least one 3′-UTR element and at least one 5′-UTR element comprises or consists of a nucleic acid sequence which is derived from the 3′-UTR, or the 5′-UTR respectively, of a human or murine protein coding gene;
(ii) the at least one 3′-UTR element, the at least one 5′-UTR element and the at least one open reading frame are all heterologous to each other;
(iii) the at least one 5′-UTR element is derived from a gene selected from the group consisting of: housekeeping genes, genes coding for a membrane protein, genes involved in cellular metabolism, genes involved in transcription, translation and replication processes, genes involved in protein modification and genes involved in cell division;
(iv) the 5′-UTR is preferably not a 5′ TOP UTR; and
(v) the 3′-UTR is preferably not derived from a gene coding for a ribosomal protein or for albumin or from the FIG. 4 gene.

More preferably, such an artificial nucleic acid molecule according to the present invention:
(i) comprises at least one 3′-UTR element and at least one 5′-UTR element, wherein preferably (each of) the at least one 3′-UTR element and at least one 5′-UTR element comprises or consists of a nucleic acid sequence which is derived from the 3′-UTR, or the 5′-UTR respectively, of a human or murine protein coding gene;
(ii) the at least one 3′-UTR element, the at least one 5′-UTR element and the at least one open reading frame are all heterologous to each other;
(iii) the at least one 3′ UTR element is derived from a human or a murine gene selected from the group consisting of: housekeeping genes, genes coding for a membrane protein, genes involved in cellular metabolism, genes involved in transcription, translation and replication processes, genes involved in protein modification and genes involved in cell division;
(iv) the 3′UTR is not derived from a gene coding for a ribosomal protein or for albumin or from the FIG. 4 gene;
(v) the at least one 5′-UTR element is derived from a human or a murine gene selected from the group consisting of: housekeeping genes, genes coding for a membrane protein, genes involved in cellular metabolism, genes involved in transcription, translation and replication processes, genes involved in protein modification and genes involved in cell division; and
(vi) the 5′-UTR is not a 5′ TOP UTR.

Thereby, it is preferred in the artificial nucleic acid molecule according to the present invention that the 3′-UTR and the 5′-UTR are derived from a human or a murine housekeeping gene. It is also preferred that the 3′-UTR and the 5′-UTR are derived from a human or a murine gene coding for a membrane protein. It is also preferred that the 3′-UTR and the 5′-UTR are derived from a human or a murine gene involved in cellular metabolism. It is also preferred that the 3′-UTR and the 5′-UTR are derived from a human or a murine gene involved in transcription, translation and replication processes. It is also preferred that the 3′-UTR and the 5′-UTR are derived from a human or a murine gene involved in protein modification. It is also preferred that the 3′-UTR and the 5′-UTR are derived from a human or a murine gene involved in cell division. In this context, the skilled person is aware that if (i) the 3′-UTR and the 5′-UTR are derived from genes belonging to the same gene class and (ii) the at least one 3′-UTR and the at least one 5′-UTR are heterologous to each other, that the 3′-UTR and the 5′-UTR are not derived from the same gene, but from distinct genes belonging to the same gene class. Accordingly, it is preferred that the at least one 3′-UTR and the at least one 5′-UTR are derived from distinct genes belonging to the same gene class.

As used herein the term “gene class” refers to the classification of genes. Examples of gene classes include (i) housekeeping genes, (ii) genes coding for a membrane protein, (iii) genes involved in cellular metabolism, (iv) genes involved in transcription, translation and replication processes, (v) genes involved in protein modification and (vi) genes involved in cell division. In other words, “housekeeping genes” is one gene class, whereas “genes involved in transcription” is another gene class, “genes involved in cellular metabolism” is a further gene class, etc.

It is also preferred in the artificial nucleic acid molecule according to the present invention as described herein, that the 3′-UTR and the 5′-UTR are derived from a human or a murine gene selected from the group consisting of: genes coding for a membrane protein, genes involved in cellular metabolism, genes involved in transcription, translation and replication processes, genes involved in protein modification and genes involved in cell division, wherein the 3′-UTR and the 5′-UTR are selected from distinct gene classes.

Preferably, the at least one 3′-UTR element and/or to the at least one 5′-UTR element is functionally linked to the ORF. This means preferably that the 3′-UTR element and/or to the at least one 5′-UTR element is associated with the ORF such that it may exert a function, such as an enhancing or stabilizing function on the expression of the encoded peptide or protein or a stabilizing function on the artificial nucleic acid molecule. Preferably, the ORF and the 3′-UTR element are associated in 5′43′ direction and/or the 5′-UTR element and the ORF are associated in 5′43′ direction. Thus, preferably, the artificial nucleic acid molecule comprises in general the structure 5′-[5′-UTR element]-(optional)-linker-ORF-(optional)-linker-[3′-UTR element]-3′, wherein the artificial nucleic acid molecule may comprise only a 5′-UTR element and no 3′-UTR element, only a 3′-UTR element and no 5′-UTR element, or both, a 3′-UTR element and a 5′-UTR element. Furthermore, the linker may be present or absent. For example, the linker may be one or more nucleotides, such as a stretch of 1-50 or 1-20 nucleotides, e.g., comprising or consisting of one or more restriction enzyme recognition sites (restriction sites).

Preferably, the at least one 3′-UTR element and/or the at least one 5′-UTR element comprises or consists of a nucleic acid sequence which is derived from the 3′-UTR and/or the 5′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), Ybx1 (Y-Box binding protein 1), Ndufb8 (NADH dehydrogenase (ubiquinone) 1 beta subcomplex 8), CNTN1 (contactin 1), LTA4H, SLC38A6, DECR1, PIGK, FAM175A, PHYH, TBC1D19, PIGB, ALG6, CRYZ, BRP44L, ACADSB, SUPT3H, TMEM14A, GRAMD1C, C11orf80, C9orf46, ANXA4, TBCK, IFI6, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, NT5DC1, RAB7A, AGA, TPK1, MBNL3, HADHB, MCCC2, CAT, ANAPC4, PCCB, PHKB, ABCB7, PGCP, GPD2, TMEM38B, NFU1, OMA1, LOC128322/NUTF2, NUBPL, LANCL1, HHLA3, PIR, ACAA2, CTBS, GSTM4, ALG8, Atp5e, Gstm5, Uqcr11, Ifi27I2a, Anapc13, Atp5I, Tmsb10, Nenf, Ndufa7, Atp5k, 1110008P14Rik, Cox4i1, Cox6a1, Ndufs6, Sec61b, Romo1, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Myeov2, Ndufs5, 1810027O10Rik, Atp5o, Shfm1, Tspo, S100a6, Taldo1, Bloc1s1, Hexa, Ndufb11, Map11c3a, Gpx4, Mif, Cox6b1, RIKEN cDNA2900010J23 (Swi5), Sec61g, 2900010M23Rik, Anapc5, Mars2, Phpt1, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, LMBRD2, TLR3, BCAT1, TOM1L1, SLC35A1, GLYATL2, STAT4, GULP1, EHHADH, NBEAL1, KIAA1598, HFE, KIAA1324L, and MANSCI.

In a particularly preferred embodiment the at least one 3′-UTR element and/or the at least one 5′-UTR element comprises or consists of a nucleic acid sequence which is derived from the 3′-UTR and/or the 5′-UTR of a transcript of a gene selected from the group consisting of GNAS (guanine nucleotide binding protein, alpha stimulating complex locus), MORN2 (MORN repeat containing 2), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), LTA4H, SLC38A6, DECR1, PIGK, FAM175A, PHYH, TBC1D19, PIGB, ALG6, CRYZ, BRP44L, ACADSB, SUPT3H, TMEM14A, GRAMD1C, C11orf80, C9orf46, ANXA4, TBCK, IFI6, C2orf34, ALDH6A1, AGTPBP1, CCDC53, LRRC28, CCDC109B, PUS10, CASP1, SNX14, SKAP2, NDUFB6, EFHA1, BCKDHB, BBS2, ITGA6, HERC5, NT5DC1, RAB7A, AGA, TPK1, MBNL3, HADHB, MCCC2, CAT, ANAPC4, PCCB, PHKB, ABCB7, PGCP, GPD2, TMEM38B, NFU1, OMA1, LOC128322/NUTF2, NUBPL, LANCL1, HHLA3, PIR, ACAA2, CTBS, GSTM4, ALG8, Atp5e, Gstm5, Uqcr11, Ifi27I2a, Anapc13, Atp5I, Nenf, Ndufa7, Atp5k, 1110008P14Rik, Cox4i1, Cox6a1, Ndufs6, Sec61b, Romo1, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Ndufs5, 1810027010Rik, Atp5o, Shfml, Tspo, S100a6, Taldol, Bloc1s1, Hexa, Ndufb11, Map11c3a, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Ldhb, Nme2, Snrpg, Ndufa2, Serf1, Oaz1, Ybx1, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, Clorf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI.

More preferably, the at least one 3′-UTR element and/or the at least one 5′-UTR element comprises or consists of a nucleic acid sequence which is derived from the 3′-UTR and/or the 5′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), Ybx1 (Y-Box binding protein 1), Ndufb8 (NADH dehydrogenase (ubiquinone) 1 beta subcomplex 8), CNTN1 (contactin 1) and NDUFA4 (NADH dehydrogenase (ubiquinone) 1 alpha subcomplex 4).

Preferably, the at least one 3′-UTR element and/or the at least one 5′-UTR element of the artificial nucleic acid molecule according to the present invention comprises or consists of a “functional fragment”, a “functional variant” or a “functional fragment of a variant” of the 3′-UTR and/or the 5′-UTR of a transcript of a gene.

Preferably, the at least one 3′-UTR element and/or the at least one 5′-UTR element comprises a nucleic acid sequence which is derived from the 3′-UTR and/or the 5′-UTR of a transcript of a human gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), LTA4H, SLC38A6, DECR1, PIGK, FAM175A, PHYH, TBC1D19, PIGB, ALG6, CRYZ, BRP44L, ACADSB, SUPT3H, TMEM14A, GRAMD1C, C11orf80, C9orf46, ANXA4, TBCK, IFI6, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, NT5DC1, RAB7A, AGA, TPK1, MBNL3, HADHB, MCCC2, CAT, ANAPC4, PCCB, PHKB, ABCB7, PGCP, GPD2, TMEM38B, NFU1, OMA1, LOC128322/NUTF2, NUBPL, LANCL1, HHLA3, PIR, ACAA2, CTBS, GSTM4, ALG8, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI.

Alternatively or additionally, it is also preferred that the at least one 3′-UTR element and/or the at least one 5′-UTR element comprises a nucleic acid sequence which is derived from the 3′-UTR and/or the 5′-UTR of a transcript of a murine gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), Atp5e, Gstm5, Uqcr11, Ifi27I2a, Anapc13, Atp5I, Tmsb10, Nenf, Ndufa7, Atp5k, 1110008P14Rik, Cox4i1, Cox6a1, Ndufs6, Sec61b, Romo1, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Myeov2, Ndufs5, 1810027010Rik, Atp5o, Shfml, Tspo, S100a6, Taldol, Bloc1s1, Hexa, Ndufb11, Map11c3a, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Ybx1, Sepp1, and Gaa.

Preferably, the at least one 3′-UTR element comprises a nucleic acid sequence which is derived from the 3′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), SLC38A6, DECR1, PIGK, FAM175A, PHYH, TBC1D19, PIGB, ALG6, CRYZ, BRP44L, ACADSB, TMEM14A, GRAMD1C, C11orf80, ANXA4, TBCK, IFI6, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, HADHB, ANAPC4, PCCB, ABCB7, PGCP, NFU1, OMA1, HHLA3, ACAA2, GSTM4, ALG8, Atp5e, Gstm5, Uqcr11, Ifi27I2a, Cbr2, Atp5I, Tmsb10, Nenf, Atp5k, 1110008P14Rik, Cox4i1, Cox6a1, Ndufs6, Sec61b, Romo1, Gnas, Snrpd2, Mgst3, Aldh2, Ss4, Myl6, Prdx4, Ubl5, 1110001J03Rik, Ndufa13, Ndufa3, Gstp2, Tmem160, Ergic3, Pgcp, Slpi, Myeov2, Ndufa4, Ndufs5, Gstml, 1810027010Rik, Atp5o, Shfml, Tspo, S100a6, Taldol, Bloc1s1, Ndufb11, Map11c3a, Morn2, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Ybx1, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI. More preferably, the at least one 3′-UTR element comprises or consists of a nucleic acid sequence which is derived from the 3′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1).

In a particularly preferred embodiment, the at least one 3′-UTR element comprises a nucleic acid sequence which is derived from the 3′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), SLC38A6, DECR1, PIGK, FAM175A, PHYH, TBC1D19, PIGB, ALG6, CRYZ, BRP44L, ACADSB, TMEM14A, GRAMD1C, C11orf80, ANXA4, TBCK, IF16, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, HADHB, ANAPC4, PCCB, ABCB7, PGCP, NFU1, OMA1, HHLA3, ACAA2, GSTM4, ALG8, Atp5e, Gstm5, Uqcr11, Ifi2712a, Cbr2, Atp5I, Tmsb10, Nenf, Atp5k, 1110008P14Rik, Cox4i1, Cox6a1, Ndufs6, Sec61b, Romo1, Gnas, Snrpd2, Mgst3, Aldh2, Ss4, Myl6, Prdx4, Ubl5, 1110001J03Rik, Ndufa13, Ndufa3, Gstp2, Tmem160, Ergic3, Pgcp, Slpi, Myeov2, Ndufa4, Ndufs5, Gstml, 1810027010Rik, Atp5o, Shfml, Tspo, S100a6, Taldo1, Bloc1s1, Ndufb11, Map11c3a, Morn2, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Ybx1, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI. More preferably, the at least one 3′-UTR element comprises or consists of a nucleic acid sequence which is derived from the 3′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1).

More preferably, the at least one 3′-UTR element comprises a nucleic acid sequence which is derived from the 3′-UTR of a transcript of a human gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), SLC38A6, DECR1, PIGK, FAM175A, PHYH, TBC1D19, PIGB, ALG6, CRYZ, BRP44L, ACADSB, TMEM14A, GRAMD1C, C11orf80, ANXA4, TBCK, IFI6, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, HADHB, ANAPC4, PCCB, ABCB7, PGCP, NFU1, OMA1, HHLA3, ACAA2, GSTM4, ALG8, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI; preferably, the at least one 3′-UTR element comprises or consists of a nucleic acid sequence which is derived from the 3′-UTR of a transcript of a human gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1).

Accordingly, it is also more preferable that the at least one 3′-UTR element comprises a nucleic acid sequence which is derived from the 3′-UTR of a transcript of a murine gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1), Ndufa1, Atp5e, Gstm5, Uqcr11, Ifi27I2a, Cbr2, Atp5I, Tmsb10, Nenf, Atp5k, 1110008P14Rik, Cox4i1, Cox6a1, Ndufs6, Sec61b, Romo1, Gnas, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Myeov2, Ndufa4, Ndufs5, Gstml, 1810027010Rik, Atp5o, Shfml, Tspo, S100a6, Taldol, Bloc1s1, Ndufb11, Map11c3a, Morn2, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Ybx1, Sepp1, and Gaa; preferably, the at least one 3′-UTR element comprises or consists of a nucleic acid sequence which is derived from the 3′-UTR of a transcript of a murine gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1).

Preferably, the at least one 5′-UTR element comprises a nucleic acid sequence which is derived from the 5′-UTR of a transcript of a gene selected from the group consisting of MP68 (RIKEN cDNA 2010107E04 gene), NDUFA4 (NADH dehydrogenase (ubiquinone) 1 alpha subcomplex 4), LTA4H, DECR1, PIGK, TBC1D19, BRP44L, ACADSB, SUPT3H, TMEM14A, C9orf46, ANXA4, IF16, C2orf34, ALDH6A1, CCDC53, CCDC104, CASP1, NDUFB6, BCKDHB, BBS2, HERC5, FAM175A, NT5DC1, RAB7A, AGA, TPK1, MBNL3, MCCC2, CAT, ANAPC4, PHKB, ABCB7, GPD2, TMEM38B, NFU1, LOC128322/NUTF2, NUBPL, LANCL1, PIR, CTBS, GSTM4, Ndufa1, Atp5e, Gstm5, Cbr2, Anapc13, Ndufa7, Atp5k, 1110008P14Rik, Cox4i1, Ndufs6, Sec61b, Snrpd2, Mgst3, Prdx4; Pgcp; Myeov2; Ndufa4; Ndufs5; Gstml; Atp5o; Tspo; Taldol; Bloc1s1; and Hexa. More preferably, the at least one 5′-UTR element comprises or consists of a nucleic acid sequence which is derived from the 5′-UTR of a transcript of MP68 (RIKEN cDNA 2010107E04 gene) or NDUFA4 (NADH dehydrogenase (ubiquinone) 1 alpha subcomplex 4).

In a particularly preferred embodiment, the at least one 5′-UTR element comprises a nucleic acid sequence which is derived from the 5′-UTR of a transcript of a gene selected from the group consisting of MP68 (RIKEN cDNA 2010107E04 gene), NDUFA4 (NADH dehydrogenase (ubiquinone) 1 alpha subcomplex 4), LTA4H, DECR1, PIGK, TBC1D19, BRP44L, ACADSB, SUPT3H, TMEM14A, C9orf46, ANXA4, IF16, C2orf34, ALDH6A1, CCDC53, CASP1, NDUFB6, BCKDHB, BBS2, HERC5, FAM175A, NT5DC1, RAB7A, AGA, TPK1, MBNL3, MCCC2, CAT, ANAPC4, PHKB, ABCB7, GPD2, TMEM38B, NFU1, LOC128322/NUTF2, NUBPL, LANCL1, PIR, CTBS, GSTM4, Ndufa1, Atp5e, Gstm5, Cbr2, Anapc13, Ndufa7, Atp5k, 1110008P14Rik, Cox4i1, Ndufs6, Sec61b, Snrpd2, Mgst3, Prdx4; Pgcp; Ndufa4; Ndufs5; Atp5o; Tspo; Taldol; Bloc1s1; and Hexa. More preferably, the at least one 5′-UTR element comprises or consists of a nucleic acid sequence which is derived from the 5′-UTR of a transcript of MP68 (RIKEN cDNA 2010107E04 gene) or NDUFA4 (NADH dehydrogenase (ubiquinone) 1 alpha subcomplex 4).

More preferably, the at least one 5′-UTR element comprises a nucleic acid sequence which is derived from the 5′-UTR of a transcript of a human gene selected from the group consisting of MP68 (RIKEN cDNA 2010107E04 gene), NDUFA4 (NADH dehydrogenase (ubiquinone) 1 alpha subcomplex 4), LTA4H, DECR1, PIGK, TBC1D19, BRP44L, ACADSB, SUPT3H, TMEM14A, C9orf46, ANXA4, IF16, C2orf34, ALDH6A1, CCDC53, CCDC104, CASP1, NDUFB6, BCKDHB, BBS2, HERC5, FAM175A, NT5DC1, RAB7A, AGA, TPK1, MBNL3, MCCC2, CAT, ANAPC4, PHKB, ABCB7, GPD2, TMEM38B, NFU1, LOC128322/NUTF2, NUBPL, LANCL1, PIR, CTBS, and GSTM4; preferably, the at least one 5′-UTR element comprises or consists of a nucleic acid sequence which is derived from the 5′-UTR of a human transcript of MP68 (RIKEN cDNA 2010107E04 gene) or NDUFA4 (NADH dehydrogenase (ubiquinone) 1 alpha subcomplex 4).

Accordingly, it is also more preferable that the at least one 5′-UTR element comprises a nucleic acid sequence which is derived from the 5′-UTR of a transcript of a murine gene selected from the group consisting of MP68 (RIKEN cDNA 2010107E04 gene), NDUFA4 (NADH dehydrogenase (ubiquinone) 1 alpha subcomplex 4), Ndufa1, Atp5e, Gstm5, Cbr2, Anapc13, Ndufa7, Atp5k, 1110008P14Rik, Cox4i1, Ndufs6, Sec61b, Snrpd2, Mgst3, Prdx4; Pgcp; Myeov2; Ndufa4; Ndufs5; Gstml; Atp5o; Tspo; Taldol; Bloc1s1; and Hexa; preferably, the at least one 5′-UTR element comprises or consists of a nucleic acid sequence which is derived from the 5′-UTR of a murine transcript of MP68 (RIKEN cDNA 2010107E04 gene) or NDUFA4 (NADH dehydrogenase (ubiquinone) 1 alpha subcomplex 4).

The phrase “nucleic acid sequence which is derived from the 3′-UTR and/or the 5′-UTR of a of a transcript of a gene” preferably refers to a nucleic acid sequence which is based on the 3′-UTR sequence and/or on the 5′-UTR sequence of a transcript of a gene or a fragment or part thereof, preferably a naturally occurring gene or a fragment or part thereof. This phrase includes sequences corresponding to the entire 3′-UTR sequence and/or the entire 5′-UTR sequence, i.e. the full length 3′-UTR and/or 5′-UTR sequence of a transcript of a gene, and sequences corresponding to a fragment of the 3′-UTR sequence and/or the 5′-UTR sequence of a transcript of a gene. Preferably, a fragment of a 3′-UTR and/or a 5′-UTR of a transcript of a gene consists of a continuous stretch of nucleotides corresponding to a continuous stretch of nucleotides in the full-length 3′-UTR and/or 5′-UTR of a transcript of a gene, which represents at least 5%, 10%, 20%, preferably at least 30%, more preferably at least 40%, more preferably at least 50%, even more preferably at least 60%, even more preferably at least 70%, even more preferably at least 80%, and most preferably at least 90% of the full-length 3′-UTR and/or 5′-UTR of a transcript of a gene. Such a fragment, in the sense of the present invention, is preferably a functional fragment as described herein. Preferably, the fragment retains a regulatory function for the translation of the ORF linked to the 3′-UTR and/or 5′-UTR or fragment thereof.

The terms “variant of the 3′-UTR and/or variant of the 5′-UTR of a of a transcript of a gene” and “variant thereof” in the context of a 3′-UTR and/or a 5′-UTR of a transcript of a gene refers to a variant of the 3′-UTR and/or 5′-UTR of a transcript of a naturally occurring gene, preferably to a variant of the 3′-UTR and/or 5′-UTR of a transcript of a vertebrate gene, more preferably to a variant of the 3′-UTR and/or 5′-UTR of a transcript of a mammalian gene, even more preferably to a variant of the 3′-UTR and/or 5′-UTR of a transcript of a primate gene, in particular a human gene as described above. Such variant may be a modified 3′-UTR and/or 5′-UTR of a transcript of a gene. For example, a variant 3′-UTR and/or a variant of the 5′-UTR may exhibit one or more nucleotide deletions, insertions, additions and/or substitutions compared to the naturally occurring 3′-UTR and/or 5′-UTR from which the variant is derived. Preferably, a variant of a 3′-UTR and/or variant of the 5′-UTR of a of a transcript of a gene is at least 40%, preferably at least 50%, more preferably at least 60%, more preferably at least 70%, even more preferably at least 80%, even more preferably at least 90%, most preferably at least 95% identical to the naturally occurring 3′-UTR and/or 5′-UTR the variant is derived from. Preferably, the variant is a functional variant as described herein.

The phrase “a nucleic acid sequence which is derived from a variant of the 3′-UTR and/or from a variant of the 5′-UTR of a of a transcript of a gene” preferably refers to a nucleic acid sequence which is based on a variant of the 3′-UTR sequence and/or the 5′-UTR of a transcript of a gene or on a fragment or part thereof as described above. This phrase includes sequences corresponding to the entire sequence of the variant of the 3′-UTR and/or the 5′-UTR of a transcript of a gene, i.e. the full length variant 3′-UTR sequence and/or the full length variant 5′-UTR sequence of a transcript of a gene, and sequences corresponding to a fragment of the variant 3′-UTR sequence and/or a fragment of the variant 5′-UTR sequence of a transcript of a gene. Preferably, a fragment of a variant of the 3′-UTR and/or the 5′-UTR of a transcript of a gene consists of a continuous stretch of nucleotides corresponding to a continuous stretch of nucleotides in the full-length variant of the 3′-UTR and/or the 5′-UTR of a transcript of a gene, which represents at least 20%, preferably at least 30%, more preferably at least 40%, more preferably at least 50%, even more preferably at least 60%, even more preferably at least 70%, even more preferably at least 80%, and most preferably at least 90% of the full-length variant of the 3′-UTR and/or the 5′-UTR of a transcript of a gene. Such a fragment of a variant, in the sense of the present invention, is preferably a functional fragment of a variant as described herein.

The terms “functional variant”, “functional fragment”, and “functional fragment of a variant” (also termed “functional variant fragment”) in the context of the present invention, mean that the fragment of the 3′-UTR and/or the 5′-UTR, the variant of the 3′-UTR and/or the 5′-UTR, or the fragment of a variant of the 3′-UTR and/or the 5′-UTR of a transcript of a gene fulfils at least one, preferably more than one function of the naturally occurring 3′-UTR and/or 5′-UTR of a transcript of a gene of which the variant, the fragment, or the fragment of a variant is derived. Such function may be, for example, stabilizing mRNA and/or enhancing, stabilizing and/or prolonging protein production from an mRNA and/or increasing protein expression or total protein production from an mRNA, preferably in a mammalian cell, such as in a human cell. Preferably, the function of the 3′-UTR and/or the 5′-UTR concerns the translation of the protein encoded by the ORF. More preferably, the function comprises enhancing translation efficiency of the ORF linked to the 3′-UTR and/or the 5′-UTR or fragment or variant thereof. It is particularly preferred that the variant, the fragment, and the variant fragment in the context of the present invention fulfil the function of stabilizing an mRNA, preferably in a mammalian cell, such as a human cell, compared to an mRNA comprising a reference 3′-UTR and/or a reference 5′-UTR or lacking a 3′-UTR and/or a 5′-UTR, and/or the function of enhancing, stabilizing and/or prolonging protein production from an mRNA, preferably in a mammalian cell, such as in a human cell, compared to an mRNA comprising a reference 3′-UTR and/or a reference 5′-UTR or lacking a 3′-UTR and/or a 5′-UTR, and/or the function of increasing protein production from an mRNA, preferably in a mammalian cell, such as in a human cell, compared to an mRNA comprising a reference 3′-UTR and/or a reference 5′-UTR or lacking a 3′-UTR and/or a 5′-UTR. A reference 3′-UTR and/or a reference 5′-UTR may be, for example, a 3′-UTR and/or a 5′-UTR naturally occurring in combination with the ORF. Furthermore, a functional variant, a functional fragment, or a functional variant fragment of a 3′-UTR and/or a 5′-UTR of a transcript of a gene preferably does not have a substantially diminishing effect on the efficiency of translation of the mRNA which comprises such variant, fragment, or variant fragment of a 3′-UTR and/or a 5′-UTR compared to the wild type 3′-UTR and/or the wild-type 5′-UTR from which the variant, the fragment, or the variant fragment is derived. A particularly preferred function of a “functional fragment”, a “functional variant” or a “functional fragment of a variant” of the 3′-UTR and/or the 5′-UTR of a transcript of a gene in the context of the present invention is the enhancement, stabilization and/or prolongation of protein production by expression of an mRNA carrying the functional fragment, functional variant or functional fragment of a variant as described above. Preferably, the efficiency of the one or more functions exerted by the functional variant, the functional fragment, or the functional variant fragment, such as mRNA and/or protein production stabilizing efficiency and/or the protein production increasing efficiency, is increased by at least 5%, more preferably by at least 10%, more preferably by at least 20%, more preferably by at least 30%, more preferably by at least 40%, more preferably by at least 50%, more preferably by at least 60%, even more preferably by at least 70%, even more preferably by at least 80%, most preferably by at least 90% with respect to the mRNA and/or protein production stabilizing efficiency and/or the protein production increasing efficiency exhibited by the naturally occurring 3′-UTR and/or 5′-UTR of a transcript of a gene from which the variant, the fragment or the variant fragment is derived.

In the context of the present invention, a fragment of the 3′-UTR and/or of the 5′-UTR of a transcript of a gene or of a variant of the 3′-UTR and/or of the 5′-UTR of a transcript of a gene preferably exhibits a length of at least about 3 nucleotides, preferably of at least about 5 nucleotides, more preferably of at least about 10, 15, 20, 25 or 30 nucleotides, even more preferably of at least about 50 nucleotides, most preferably of at least about 70 nucleotides. Preferably, such fragment of the 3′-UTR and/or of the 5′-UTR of a transcript of a gene or of a variant of the 3′-UTR and/or of the 5′-UTR of a transcript of a gene is a functional fragment as described above. In a preferred embodiment, the 3′-UTR and/or the 5′-UTR of a transcript of a gene or a fragment or variant thereof exhibits a length of between 3 and about 500 nucleotides, preferably of between 5 and about 150 nucleotides, more preferably of between 10 and 100 nucleotides, even more preferably of between 15 and 90, most preferably of between 20 and 70. Typically, the 5′-UTR element and/or the 3′-UTR element is characterized by less than 500, 400, 300, 200, 150 or less than 100 nucleotides.

Preferably, the at least one 3′-UTR element comprises or consists of a nucleic acid sequence which has an identity of at least about 1, 2, 3, 4, 5, 10, 15, 20, 30 or 40%, preferably of at least about 50%, preferably of at least about 60%, preferably of at least about 70%, more preferably of at least about 80%, more preferably of at least about 90%, even more preferably of at least about 95%, even more preferably of at least about 99% to a nucleic acid sequence selected from the group consisting of SEQ ID NOs: 1 to 24 and SEQ ID NOs: 49 to 318 or the corresponding RNA sequence, respectively, or wherein the at least one 3′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to a nucleic acid sequence selected from the group consisting of SEQ ID NOs: 1 to 24 and SEQ ID NOs: 49 to 318 or the corresponding RNA sequence, respectively:

Homo sapiens SLC38A6 3′-UTR
SLC38A6-001 ENST00000267488
(SEQ ID NO: 49)
AAGAAATATTTTCCTACTTCTTACAAGAATAATATACCCCTAGTTGCAAGAATGAATTATTCCGGA
AGACACCCTGGATGAAAAATAACATTTTAATAAAAATTATTAACAGAAAAGCAGAACAAAATGGCA
GTGGGTATGGGGAAGTAAGAGTGTGGCAGTTTTAATCAAAAAAAGAAACAAACTCGAAATGCTCTT
AAA
Homo sapiens DECR1 3′-UTR
NM_001359.1
(SEQ ID NO: 50)
GACCACTTTGGCCTTCATCTTGGTTACAGAAAAGGGAATAGAAATGAAACAAATTATCTCTCATCT
TTTGACTATTTCAAGTCTAATAAATTCTTAATTAAC
Homo sapiens PIGK 3′-UTR
(SEQ ID NO: 51)
ACTTGATGATGAATGAAGAATGCATGGAGGACTGCAAACTTGGATAATAATTTATGTCATTATATA
TTTTTAAAAATGTGTTTCTCTTGTATGAATTGGAAATAAGTATAAGGAAACTAAATTTGAATCAAC
TATTAATTTTATAACTTAAAGAAAAATAATTGTTAATGCAACTGCTTAATGGCACTAAATATATTC
CAGTTTTGTATTTTGTGTATTATAAAAGCGAATGAGACAGAGATCAGAATACATTGACTGTTTTTG
AAAATAGTAATTTCCCCTTATCCCCTTTTCATTTGGAAAAGAAACAATTGTGAAGACATTAAATTC
TCACTAACAGAAGTAACTTTGGTTAATTATTTTTTGTAT
Homo sapiens FAM175A 3′-UTR
FAM175A-009 ENST00000506553
(SEQ ID NO: 52)
TCCTTTTAACCTTACAAGGAGATTTTTTTATTTGGCTGATGGGTAAAGCCAAACATTTCTATTGTT
TTTACTATGTTGAGCTACTTGCAGTAAGTTCATTTGTTTTTACTATGTTCACCTGTTTGCAGTAAT
ACACAGATAACTCTTAGTGCATTTACTTCACAAAGTACTTTTTCAAACATCAGATGCTTTTATTTC
CAAACCTTTTTTTCACCTTTCACTAAGTTGTTGAGGGGAAGGCTTACACAGACACATTCTTTAGAA
TTGGAAAAGTGAGACCAGGCACAGTGGCTCACACCTGTAATCCCAGCACTTAGGGAAGACAAGTCA
GGAGGATTGATTGAAGTTAGGAGTTAGAGACCAGCCTGGGCAACGTATTGAGACCATGTCTATTAA
AAAATAAAATGGAAAAGCAAGAATAGCCTTATTTTCAAAATATGGAAAGAAATTTATATGAAAA
Homo sapiens PHYH 3′-UTR
PHYH-002 ENST00000396913
(SEQ ID NO: 53)
AATAGCCATCTGCTATAACTCTTTCAACAGAAAACCAAAACCAAACGAAATGTCTAAGGAAAATGT
TTTCTTAATGAGATGATGTAACCTTTTCTATCACTTGTTAAAAGCAGAAAACATGTATCAGGTACT
TAATTGCATAGAGTTAGTTTTGCAGCACAATGGTGTTGCTTTAATGGAAAAAAAAAACAGTAAAAG
TGAAATATTACTGTTTTAAGGAAAACTAATTTAGGGTGGCAGCCAATAAAGGTGGTTGGTGTCTAA
TTTAAGTGTTAAATCAATTTCTTTCATTCAGTTAGCTCTTTACCCAAGAAGAAGTGAATGATTTGG
AGCTTAGGGTATGTTTTGTATCCCCTTTCTGATAAACCCATTCCCTACCAATTTTATGTCATAAGA
GATTTTTTTCCCCCAAATCTAGAACAATGTATAATACATTCACATCTAGTCAAGGGCATAGGAACG
GTGTCATGGAGTCCAAATAAAGTGGATATTCCTGCTCGGACAA
Homo sapiens TBC1D19 3′-UTR
TBC1D19-001 ENST00000264866
(SEQ ID NO: 54)
TCTTCTTCACAGTCACTGGCAACACATCTAGTTTTTCATTAGAAACAAATCATGAACTATGCAAAC
TCTGCATAAAACCAAAATGAAACTTTGCATATAAGCCAATAAAGATCATGTTCCCTCTTCAGTTAA
ACCTAAGTAGTTTCTCACTTTTTGAAACAATAACTCTGCACCAAATATTGCATCGCATGCTGCTGA
TTTTCAAGAGAGAAGCAATAAACACAACTTCTGCTAAATTGAGCATTATATATATAATATTATAAT
ATATATATAATCCTGACTTGTCAATGGCATGTAATAATATATGCAATAAGAACTAAAGATACTGTA
ATAAACTTCAAGAGGTAATGTAGCTTCTTGGATAATTCTTTTATGTCAGTTTATAAATTTATCTCT
AGATAATG
Homo sapiens TBC1D19 NM_018317.2 3′-UTR
(SEQ ID NO: 55)
TCTTCTTCACAGTCACTGGCAACACATCTAGTTTTTCATTAGAAACAAATCATGAACTATGCAAAC
TCTGCATAAAACCAAAATGAAACTTTGCATATAAGCCAATAAAGATCATGTTCCCTCTTCAGTTAA
ACCTAAGTAGTTTCTCACTTTTTGAAACAATAACTCTGCACCAAATATTGCATCGCATGCTGCTGA
TTTTCAAGAGAGAAGCAATAAACACAACTTCTGCTAAATTGAGCATTATATATATAATATTATAAT
ATATATATAATCCTGACTTGTCAATGGCATGTAATAATATATGCAATAAGAACTAAAGATACTGTA
ATAAACTTCAAGAGGTAAAAAAA
Homo sapiens PIGB 3′-UTR
PIGB-201 ENST00000539642
(SEQ ID NO: 56)
AAATTCAACATGAAGATGAAATTCTGAACTTTCCTAGATAAATTAACATTGCTGGGTGGAAATATT
CAGATGCTGCTTAAATACTTCGGTAAACACTGGGTAAGATTCATGGAACTTAGAAAAAAGCTGTAT
GAACTGCTTTACCAAATATCACTACTGAGGAAATGTATAAAATACCACATAGTATAAAATTACATG
TTAATACAATGCCAGATTTTAAATAAAGACCTTTAGTTTTCCTC
Homo sapiens ALG6 3′-UTR
ALG6-006 ENST00000263440
(SEQ ID NO: 57)
CTGTATTCCTAAACAAATTGTTTCCTAAACAAATGTGAAAATGTGAACAGTGCTGAAAGGTTTTGT
GAACTTTTTGCTATGTATAAATGAAATTACCATTTTGAGAACCATGGAACCACAGGAAAGGAAATG
GTGAAAAGTCATTGTTGTCTACACA
Homo sapiens CRYZ 3′-UTR
CRYZ-005 ENST00000370871
(SEQ ID NO: 58)
TGATTAATTCTTTCATGGATTTCCTATGTAATTAGAGGTACTGTCTTTCCCCCAGTTGTACTTACC
CTATCTTTTCTTTAATTAACATTCGATTCCATGAGCTTCTTATGTGAAAAAATAAGATTTTTCTTT
AGAGAGCAGAAGCAGAAGAGTAAAATTTATTGTATAGCTAGCAATATTTTTTTATGCCATCTGTCT
CAAATCAAAGAGTCATCATAGTAGGAAATAACATGTTAGTTGTCATTTGGCATGAGTGTGCATTCC
AGTAATTCTTAATTGATATTTGATTAATTCCATACCTTTGATTAAAACATGCTAGTTCAA
Homo sapiens BRP44L 3′-UTR
BRP44L-001 ENST00000360961
(SEQ ID NO: 59)
CAATGGAAAAGGAAGAACAAGGTCTTGAAGGGACAGCATTGCCAGCTGCTGCTGAGTCACAGATTT
CATTATAAATAGCCTCCCTAAGGAAAATACACTGAATGCTATTTTTACTAACCATTCTATTTTTAT
AGAAATAGCTGAGAGTTTCTAAACCAACTCTCTGCTGCCTTACAAGTATTAAATATTTTACTTCTT
TCCATAAAGAGTAGCTCAAAATATGCAATTAATTTAATAATTTCTGATGATGGTTTTATCTGCAGT
AATATGTATATCATCTATTAGAATTTACTTAATGAAAAACTGAAGAGAACAAAATTTGTAACCACT
AGCACTTAAGTACTCCTGATTCTTAACATTGTCTTTAATGACCACAAGACAACCAACAGCTGGCCA
CGTACTTAAAATTTTGTCCCCACTGTTTAAAAATGTTACCTGTGTATTTCCATGCAGTGTATATAT
TGAGATGCTGTAACTTAATGGCAATAAATGATTTAAATATTTGTTAAA
Homo sapiens ACADSB 3′-UTR
ACADSB-004
(SEQ ID NO: 60)
CGTCTATAGGAGTGGGACCCCTCCCTGGTGTCACTGCTGTAAAATTTTAAACGGTTGTGTCTTGTT
GGGAGTAAGTGCCTTGCGTGGGAATAAACTTCCACAGCATTCGAATATTTTAATGAAGCCCTTAGT
CAGGGTCCTGGTGTTGGCCTTTTTGGTTTTCTCTTTTCAGGCTGTTTAACTTAGGCACAGGAGATC
CACTTTTAAACTTGGGAAATAAGCACCTGTATTTTTTTCCAAAACTGTTTTTAAAGCTGTATACGC
ATACATATATATATTTTTACTCTGTCTTACTCTGTCACCCAGGCTAGAGTGCAGTGGCGCGATCTC
AGCTCACTGCAGCCTTGACCTCCT
Homo sapiens TMEM14A 3′-UTR
NM_014051.3
(SEQ ID NO: 61)
GCATCTGGAGGAACAGAAAACTAAGTTCATGTCATCCTGCTGTAATGGGCAGAGCATATTTTTTTT
GTATTTAAAAGATAAACTTCAATATGGAATGCTAGAAACACAAATAGCACTGTCACCTCTAATATG
AACATTAGTTTGAGGTAGTTTTTTTCTAAAGCAAAAATTTTAACTGTTTTCTAATTGTCAAGCACT
ATTTTCATTAAAAGTGTCTAATGAATCATGATATACTCTTCCATTTGTTGTGTCTATTTTTTATAT
ATTTGGTATTTTTTGAAAATTCCAAATACTCATGTCTCAAGTAAGCTTAAACTACAACTTGTCACA
TAAAGGAAGTCTTAAGTGGAGTTCACAGAATGATAATGTATCTATTTGTCATTTGTGTTATATTTG
AAATTATTAGAAATTATGCTTTTTCCATTTTAATTGTATTGCTGCCAGTGCTATTTTTTTCTTTAA
AAAATTTTATTCTTAGCACACTGTTATGTCCTAACTGAATGTATTCAGTATTCAAATAAAAGACAT
TTTGGTTCAAA
Homo sapiens GRAMD1C 3′-UTR
GRAMD1C-005 ENST00000472026
(SEQ ID NO: 62)
TGATCTGAAGGACTAAAACCGCAGAGATACTTGGAACTTAAAGAAAATACCTGGAAGAAAACCAGA
CGAATGAAGGATTTTGGCATAGAACATTTCTATGTTTTTTCATTATTGAGATTTCTAATATGAACA
TTTCTTTCAGTAACATTTATTTGATAATTAGTTTCTGCTGGCCTTAATAATCCATCCTTTCACTTC
TTATAGATATTTTTAAGCTGTGAATTTCTTCAGTGAACCATGAAATATATTATAGAACTGAATTTC
TCTGATACAAAAAGAAAATGACACACCC
Homo sapiens C11orf80 3′-UTR
C11orf80-201 ENST00000360962
(SEQ ID NO: 63)
GCCGGGTCCCCTTCCGCAAGCGCCCACCGATCCGGAGGCTGCGGGCAGCCGTTATCCCGTGGTTTA
ATAAAGCTGCCGCGCGCTCACCAAGTCC
Homo sapiens ANXA4 3′-UTR
ANXA4-002 ENST00000409920
(SEQ ID NO: 64)
AATAAAAATCCCAGAAGGACAGGAGGATTCTCAACACTTTGAATTTTTTTAACTTCATTTTTCTAC
ACTGCTATTATCATTATCTCAGAATGCTTATTTCCAATTAAAACGCCTACAGCTGCCTCCTAGAAT
ATAGACTGTCTGTATTATTATTCACCTATAATTAGTCATTATGATGCTTTAAAGCTGTACTTGCAT
TTCAAAGCTTATAAGATATAAATGGAGATTTTAAAGTAGAAATAAATATGTATTCCATGTTTTTAA
AA
Homo sapiens TBCK 3′-UTR
TBCK-002 ENST00000361687
(SEQ ID NO: 65)
AGAACCAAGAGTGTGACTGCCAAAACTTAGTGTGGCATCAGCACCAACAGCACAGTTCTTCATATC
CACGCCACTCTCAGACAAAACTAGATGTCCAGATTGTTGCATTTCCGTAAAGTTTGTCACGAGACA
TTTTTTAAAATCTCATAACCCACATGTTCAGTTATCCATGCAAGAAACTTGACTCTACATGTATTG
CTGAAAGAATTTTCTTAACAGTGAAATCTGATCATATATTTTTACCACACTGCCACATAAAGCCCA
AGAAATTCAGCTGACAAGACAGATTTAGCATTATCAAGAAATCCCATTTGCCCTGAAAAAGCTGTC
CTCCATTGTACTGAACAGACAGTCCTGTCGATTGTGTTATTTAGAAACATACACTGAATGTGGGCT
GAAATCATCATCTTTCCATAATGAAAACTGAGAAACTATTCACAATGCATTCCTTATAAATAAATG
CTACATTTAGTAACTCATTTCACCCAAA
Homo sapiens IFI6 3′-UTR
IFI6-001 ENST00000361157
(SEQ ID NO: 66)
CCAGCAGCTCCCAGAACCTCTTCTTCCTTCTTGGCCTAACTCTTCCAGTTAGGATCTAGAACTTTG
CCTTTTTTTTTTTTTTTTTTTTTTTGAGATGGGTTCTCACTATATTGTCCAGGCTAGAGTGCAGTG
GCTATTCACAGATGCGAACATAGTACACTGCAGCCTCCAACTCCTAGCCTCAAGTGATCCTCCTGT
CTCAACCTCCCAAGTAGGATTACAAGCATGCGCCGACGATGCCCAGAATCCAGAACTTTGTCTATC
ACTCTCCCCAACAACCTAGATGTGAAAACAGAATAAACTTCACCCAGAAAACACTT
Homo sapiens CAMKMT 3′-UTR
(synonym C2orf34) ENST00000378494
(SEQ ID NO: 67)
AAGATTAAGCTTCTCAAAGACGAAGAAACGTATCAAGTGCATAGGGAATATTTTTACAAAAACGGA
AATCTGTAAGGGGTATAATCGCCTGCCTGCGCCCTTTGCAGCATTTCACGTGTGGGCTATGGACTC
CACCTGTCCTCACCCACGTTATTCCCCAGCTGCCCTCTCCAGCTCCCTCCCCGCCTCTTTTTACAC
TCTGCTTGTTGCTCGTCCTGCCCTAAACCTTTGTTTGTCTTTAAATGTGTATAAGCTGCCTGTCTG
TGACTTGAATTTGACTGGTGAACAAACTAAATATTTTTCCCTGTAATTGAGACAGAATTTCTTTTG
ATGATACCCATCCCTCCTTCATTTTTTTTTTTTTTTTGGTCTTTGTTCTGTTTTGGTGGTGGTAGT
TTTTAATCAGTAAACCCAGCAAATATCATGATTCTTTCCTGGTTAGAAAAATAAATAAAGTGTATC
TTTTTATCTCCCTCCAA
Homo sapiens ALDH6A1 3′-UTR
NM_005589.2
(SEQ ID NO: 68)
AAACAAGTTTGTTTAAGACTGACTCCATCCTGAGTAATCTCCCTTTATTTTTGACCAGCTTCATTT
GTCAGCTTTGCTCAGATCAGATCGATGGGATTGGAATACATTGTAACTAAAATCTTCCTCAGGACT
ATTAACCCCCGCAAAGTTTCTATAGGGAACTGCCTAGTGTAACAATGAAACCAGATTTCTCACTTG
CTCTTCATACTTCTATTTTGAGGTAACTGTTGTAACTATGAAATGCTTATCTGAAAGTAGTGCTTA
AACCTGATTTCTAAAAATTATCCCATTTTCTGATGATTTGAAGGGGAGAAAAGCCAGTGTATGTAA
AGAAAATGTTCCAGCCAGGCGCGGTGGCTCACGCCTGTAATTCCATCATTTTGGGAGGCCACAGTG
GGCAGATTGCTTGAGCCCAGGAGTTGAAGAACGTGGCGAAACCCCGTATCTATTATTTAAAAAAAT
TGAAAAAGTAAAAA
Homo sapiens AGTPBP1 3′-UTR
AGTPBP1-004 ENST00000357081
(SEQ ID NO: 69)
GCCCGCTGCCATCTCTTGTTAACTGCAAAGAATAAATGAAATATCTTGGTTTTTATTTCCCAGGAA
GCTTGAGAGAAATGAGTTTATACAGAGCTGACTCAAAAAGACAAAAAGTAACTTGGGCCAGTTTGG
TTTCAAGATAATAAATGTGTTATTAATTAATGATAAAATTGGCGCTTGTTTTATTTTCGATATTCA
ATGCACTTTATGTAGCATTGAATGATCAAATATTGGATTTACCTTTAAAAAAAAAACCTGAGTATC
ATTGCATGAATTTTTATCTCCCTATGGTTATATCCTGCATCAAGTGGATAATTTTGAAGTGTGTTC
AGAATATAAAATTGAAATTTTAGAGTTGTTGAAAATCCTGACTTGTTGAAAACTAATATATATGTA
CATGGATTTCTATAGATGTGTTTGTTTAGAAGTGGGTAGATATTGCAGATAAGACTGTTCTTCAGA
ATCATGTTAACTATTGGGTTGTGACTGAAGTAGTCCAGGGTTTGCCTTGAAACCATTACATTCTAC
ATTTACCAAATTAAACAAATAAAAACTGTATTAAATGTT
Homo sapiens CCDC53 3′-UTR
CCDC53-001 ENST00000240079
(SEQ ID NO: 70)
GCTTAATTTTGATAAGAATTACATATGCATGCATAGGGGTACATTTACATTCTGTAAGAGATTGAG
CCTGAACTCTCTTAGTCATAAAAACATCAAATGGCCACATGTCCACTACCAAGCTTCTTCTATGTT
AAAAAAATAATAATAAAGCAGTTTTAACCTGCCAGTA
Homo sapiens LRRC28 3′-UTR
LRRC28-002 ENST00000331450
(SEQ ID NO: 71)
TAAACACTCAAGAACCTCAGGAGCGCTGCCAGCTTGACACTGGGGAATCCAGCCAGTCCAGCACAC
TCTTCCATCCTGTCCTGTCCAATGCGGGGGCACTGCAGAACTCTCTAGAAATGTCATGATTGAGCT
TCAGAGCTAAAATGCCTTCACCCTTCCCCCAAGTTGGAATATATCCTCCCCCAAATTAAGGA
Homo sapiens CCDC109B 3′-UTR
NM_017918.4
(SEQ ID NO: 72)
TCTTACAGTTTTAAATGTCGTCAGATTTTCCATTATGTATTGATTTTGCAACTTAGGATGTTTTTG
AGTCCCATGGTTCATTTTGATTGTTTAATCTTTGTTATTAAATTCTTGTAAAAC
Homo sapiens PUS10 3′-UTR
PUS10-001 ENST00000316752
(SEQ ID NO: 73)
CTTTCAAATTTGGAGACAAAGAGTATGGTTTTCCTGGCATGATGTGGACATCCATGGAGCACATGC
CGTAAAATGGCTGTTTACCCACCATAACGGTGTCTTGAAAACTATTTGGATCATGTTGATCTATAT
AATTGTTAATTTGTTGTAACATCTCAGGATCTATATATGTGTATATTTTGTGTTAAATTGTTCCAA
GGATGTCTTAGGATTTTTCTCATTCCCTCTTTCACCCCCACAAACCAAACTATGAATAATGAAATA
ATTCTCCTTAATTCTTTCATTTAGAGAGGTGCACAAACAGGACACATTCTCTGTTAACCTAAGAAG
CTGTAATTTCAGCAAGATTTCCCTCCACAAGAGATATACCACCTTTAAAATCATGTTCTAATTTTT
GTAAATTATCTGAATAAAAGTTATATCTAG
Homo sapiens CCDC104 3′-UTR
CCDC104-002 ENST00000339012
(SEQ ID NO: 74)
TAATTAAGAACAATTTAACAAAATGGAAGTTCAAATTGTCTTAAAAATAAATTATTTAGTCCTTAC
ACTGA
Homo sapiens CASP1 3′-UTR
CASP1-007 ENST00000527979
(SEQ ID NO: 75)
AATAAGGAAACTGTATGAATGTCTGTGGGCAGGAAGTGAAGAGATCCTTCTGTAAAGGTTTTTGGA
ATTATGTCTGCTGAATAATAAACTTTTTTGAAATAATAAATCTGGTAGAAAAATGAAAA
Homo sapiens SNX14 3′-UTR
SNX14-007 ENST00000513865
(SEQ ID NO: 76)
ACACTTGGATTTGGTATAGAATAACCCATTGAAATTTCTGCTGTGCGAGGGTGGTAGAAATTTACT
TTTTTGGGTATATTCTTATATATATTATGTACATCGCTGTCTGAAATTTTAGTTATTTTTTGTTTT
TAATAAAGACTAACACAAACTTAATGATTAAAAGTGATTGAG
Homo sapiens SKAP2 3′-UTR
SKAP2-201 (part of SKAP2.001 ENST00000345317)
(SEQ ID NO: 77)
GAGTCCTGGAAAAGGAAAATTCTTCTGCTTGTCTGCAAATGCTTTGGATTTAGAAGCGTCATGAAA
GCACGAGTGACAGCTCCTAACCTCTCCTTGTTTTATTAAACATTACTTATCTTTGACTGTTATTTT
ATGCAGTCGCTCATTAAAATATTCCTCTGATGTGAAATTAAATGAAGGATATTAATGTAAATTAGA
TGCAACCAGTTAAGTTATACCTGTTGCTATTTTGCAAAG
Homo sapiens NDUFB6 3′-UTR
NM_182739.2
(SEQ ID NO: 78)
AGATTATGTAAAAAGTTAAAAGGCTTATGAGCCTAAGTTTGTTCCTATATTACCATATTTACTGAA
TTTTCTGGAAAAGTAACTTTAATAAAGTTTAATCTCAGAAATTGTCATATCTGTTTTCAAGCATTG
TACAATTTGAGACTGAGTAATTTAACAATAAGTAAAAAGTGGACATGCTAAACAAATATGAGAGAC
TACCTACTTTTTCTGGTCATTCTTGACTTGGAAAACGGTATGGAAAAGTATTTAGTTACATGTTTG
TTTGTTTTTTTCTTACACAGTACTTACACTAATTTGGTATCAGGGTATGCAACAGTGAAATATCAC
AATAAACAAATGTAAGAACAAAAAAAAAAAAA
Homo sapiens EFHA1 3′-UTR
EFHA1-001 ENST00000382374
(SEQ ID NO: 79)
TAAAAGATATAATAGTATGGCAATTATATTGTTCCAAATGTCAAAATTTGTGATTTTTTAGAAGTA
CTTGCTATTTATCTTCTTAAGTCTTCATTGATATTCTGTGTGAAATAAGCATGTCTTGTACTTGCT
TTCTGATTCATAATTTTATTAAAGAACTTAGTAGAAAGAAAAGTAAGTATAAAAATAGATATTGGA
TTCTGTCAGAAGGCCTAGATTTGAAATAATGTTTTGTACTTCGGTAAGATGGAAAACTTAGTGATT
CACTGATTTCTTAGACACTCTAATATGATATGCTTTCTGGAAGGATAAAACAAATACATATGGGAA
AAAGTACTTGAGACCAAGGCCAGCATCAATTCCAGACATCTTCATGTTCCTAATAGGCTAAATGAA
GTTAAAAACTTATTTCAGATTTTTCTCATCTGTACCTTATATCTCATAAATTTATTGCATATTTTA
TGTCAGTAGCTTAGCTGTTTATTGTCTTTAAAATAACATGTAAACTTCAATGTTCTATCTGGAAGC
AGAATAAAATATTTACATAGA
Homo sapiens BCKDHB 3′-UTR
BCKDHB-005 ENST00000356489
(SEQ ID NO: 80)
CCATATAGAAAAGCTGGAAGATTATGACTAGATATGGAAATATTTTTTCTGAATTTTTTTTTATAT
TTCCTCCGACTTACCTCTTTTTGAAAAGAGAGTTTTTATTAAGTGAACCATCACGATATTGGCTGA
AAAGTTCTACATTCTATTATTGTATTGTAACACACATGTATTGATGATTTTCATTAAGAGTTTCAG
ATTAACTTTGAAAAATATTCCACATGGTAATCTTATAAATTCTGTTTAATTACATCTGTAAATATT
ATGTGTGTGATAGTATTCAATAAA
Homo sapiens BCKDHB 3′-UTR
NM_001164783.1
(SEQ ID NO: 81)
GACCTGCTCAGCCCACCCCCACCCATCCTCAGCTACCCCGAGAGGTAGCCCCACTCTAAGGGGAGC
AGGGGGACCTGACAGCACACCACTGTCTTCCCCAGTCAGCTCCCTCTAAAATACTCAGCGGCCAGG
GCGGCTGCCACTCTTCACCCCTGCTCCTCCCGGCTGTTACATTGTCAGGGGACAGCATCTGCAGCA
GTTGCTGAGGCTCCGTCAGCCCCCTCTTCACCTGTTGTTACAGTGCCTTCTCCCAGGGGCTGGGTG
AGGGCACATTCAGGACTAGAAGCCCCTCTGGGCATGGGGTGGACATGGCAGGTCAGCCTGTGGAAC
TTGCGCAGGTGCGAGTGGCCAGCAGAGGTCACGAATAAACTGCATCTCTGCGCCTGGCTCTCTACC
AAAAAAAAAAAAAAAAAA
Homo sapiens BBS2 3′-UTR
NM_031885.3
(SEQ ID NO: 82)
GTGAGGAAAATACAGGTCATGAAGTTCCTGGCAAAGATTTTCTGTTAAAAACCTATGCTGGTTTGC
TTTGGATCACACCCTGGTGAACCCCGGGTGCTAAGAATGAAAATAACCTTGGTGAGTTGTACAAAT
TAAAGACAAAGAACTACATGTGAAGATAGACTTGCTTTCTATTTTTAAATCAGTAGTAGTACTGTT
GCTGAATAATACTAGGTTTTTATGGAATAGGATGAATGCTTTTGAAGTATTAGGGCTTCAGAGTCC
AATTTTGCTTATTTATGGTATATAAATACATATTTTTTTCTTGAAATTGCAATTGAGTTTGTACTT
TTCAAATAGATTATCTACTTTTTCATTAAAATGTAAAGATGTTAAACTTTGTGTTGATTGATTATA
AAATCACCACCAAATCAG
Homo sapiens LMBRD1 3′UTR
NM_018368.3
(SEQ ID NO: 83)
CAGCCTTCTGTCTTAAAGGTTTTATAATGCTGACTGAATATCTGTTATGCATTTTTAAAGTATTAA
ACTAACATTAGGATTTGCTAACTAGCTTTCATCAAAAATGGGAGCATGGCTATAAGACAACTATAT
TTTATTATATGTTTTCTGAAGTAACATTGTATCATAGATTAACATTTTAAATTACCATAATCATGC
TATGTAAATATAAGACTACTGGCTTTGTGAGGGAATGTTTGTGCAAAATTTTTTCCTCTAATGTAT
AATAGTGTTAAATTGATTAAAAATCTTCCAGAATTAATATTCCCTTTTGTCACTTTTTGAAAACAT
AATAAATCATCTGTATCTGTGCCTTAGGTTCTCCAGAGTGATGTGGAATTTTAAAGTGTCTCTCTC
TGATTGCCTCCAA
Homo sapiens ITGA6 3′-UTR
ITGA6-003 ENST00000409532
(SEQ ID NO: 84)
TATTGATCTACTTCTGTAATTGTGTGGATTCTTTAAACGCTCTAGGTACGATGACAGTGTTCCCCG
ATACCATGCTGTAAGGATCCGGAAAGAAGAGCGAGAGATCAAAGATGAAAAGTATATTGATAACCT
TGAAAAAAAACAGTGGATCACAAAGTGGAACGAAAATGAAAGCTACTCATAGCGGGGGCCTAAAAA
AAAAAAGCTTCACAGTACCCAAACTGCTTTTTCCAACTCAGAAATTCAATTTGGATTTAAAAGCCT
GCTCAATCCCTGAGGACTGATTTCAGAGTGACTACACACAGTACGAACCTACAGTTTTAACTGTGG
ATATTGTTACGTAGCCTAAGGCTCCTGTTTTGCACAGCCAAATTTAAAACTGTTGGAATGGATTTT
TCTTTAACTGCCGTAATTTAACTTTCTGGGTTGCCTTTATTTTTGGCGTGGCTGACTTACATCATG
TGTT
Homo sapiens HERC5 3′-UTR
HERC5-001 ENST00000264350
(SEQ ID NO: 85)
CCAGCTTGCTTGTCCAACAGCCTTATTTTGTTGTTGTTATCGTTGTTGTTGTTGTTGTTGTTGTTG
TTTCTCTACTTTGTTTTGTTTTAGGCTTTTAGCAGCCTGAAGCCATGGTTTTTCATTTCTGTCTCT
AGTGATAAGCAGGAAAGAGGGATGAAGAAGAGGGTTTACTGGCCGGTTAGAACCCGTGACTGTATT
CTCTCCCTTGGATACCCCTATGCCTACATCATATTCCTTACCTCTTTTGGGAAATATTTTTCAAAA
ATAAAATAACCGAAAAATTAA
Homo sapiens HADHB 3′-UTR
HADHB-001 ENST00000317799
(SEQ ID NO: 86)
TAGATCCAGAAGAAGTGACCTGAAGTTTCTGTGCAACACTCACACTAGGCAATGCCATTTCAATGC
ATTACTAAATGACATTTGTAGTTCCTAGCTCCTCTTAGGAAAACAGTTCTTGTGGCCTTCTATTAA
ATAGTTTGCACTTAAGCCTTGCCAGTGTTCTGAGCTTTTCAATAATCAGTTTACTGCTCTTTCAGG
GATTTCTAAGCCACCAGAATCTCACATGAGATGTGTGGGTGGTTGTTTTTGGTCTCTGTTGTCACT
AAAGACTAAATGAGGGTTTGCAGTTGGGAAAGAGGTCAACTGAGATTTGGAAATCATCTTTGTAAT
ATTTGCAAATTATACTTGTTCTTATCTGTGTCCTAAAGATGTGTTCTCTATAAAATACAAACCAAC
GTGCCTAATTAATTATGGAAAAATAATTCAGAATCTAAACACCACTGAAAACTTATAAAAAATGTT
TAGATACATAAATATGGTGGTCAGCGTTAATAAAGTGGAGAAATATTGGAGAA
Homo sapiens ANAPC4 3′-UTR
ANAPC4-001 ENST00000315368
(SEQ ID NO: 87)
TCTAGCTTGCCATTATTGTGTGTGTAATTATGGCCAAAAGGACATAGGAGATGGACTAAGATGTCT
TGGACCACCTTTGTGTAACAAAGAAATAAACAGTAAATTTTATTTTTTCA
Homo sapiens PCCB 3′-UTR
NM_000532.4
(SEQ ID NO: 88)
ACAAATCAAAGGAAAAGAAACCAAGAACTGAATTACTGTCTGCCCATTCACATCCCATTCCTGCCT
TTTGCAATCATGAAACCTGGGAATCCAAATAGTTGGATAACTTAGAATAACTAAGTTTATTAAATT
CTAGAAAGATCTCAAAAAAAAA
Homo sapiens ABCB7 3′-UTR
ABCB7-001 ENST00000253577
(SEQ ID NO: 89)
GTCACATAAGACATTTTCTTTTTTTGTTGTTTTGGACTACATATTTGCACTGAAGCAGAATTGTTT
TATTAAAAAAATCATACATTCCCA
Homo sapiens PGCP 3′-UTR
CPQ-001 ENST00000220763
(SEQ ID NO: 90)
AAACAGTAAGAAAGAAACGTTTTCATGCTTCTGGCCAGGAATCCTGGGTCTGCAACTTTGGAAAAC
TCCTCTTCACATAACAATTTCATCCAATTCATCTTCAAAGCACAACTCTATTTCATGCTTTCTGTT
ATTATCTTTCTTGATACTTTCCAAATTCTCTGATTCTAGAAAAAGGAATCATTCTCCCCTCCCTCC
CACCACATAGAATCAACATATGGTAGGGATTACAGTGGGGGCATTTCTTTATATCACCTCTTAAAA
ACATTGTTTCCACTTTAAAAGTAAACACTTAATAAATTTTTGGAAGATCTCTGA
Homo sapiens NFU1 3′-UTR
NM_001002755.2
(SEQ ID NO: 91)
AATAATCTGGATTTTCTTTGGGCATAACAGTCAGACTTGTTGATAATATATATCAAGTTTTTATTA
TTAATATGCTGAGGAACTTGAAGATTAATAAAATATGCTCTTCAGAGAATGATATATAAATATTGC
A
Homo sapiens OMA1 3′-UTR
OMA1-001 ENST00000371226
(SEQ ID NO: 92)
ATTAAAATTTATGAGACACAAGATATATGAAGAATGTTGCAGTCCTTATCATTTTATGTTACTTTT
TAAAAAATGATGTTTGAAGTGAAAAAAAAAAGGATATTCAGGGTCAAATCATGTACATTACAGATA
TTATCTAAATTCTTCTAGAATTTATTTTTCATGAAATATTGATGTATTTTAATCTATGTTAAAATA
TCTTCAATGAGGAAAATGTCACAGAATAAATTTATATTACACATTTTA
Homo sapiens HHLA3 3′-UTR
NM_001036646.1
(SEQ ID NO: 93)
GGCGAATCCATAGAGTAAGCTTAGTGATGTGTGTCAGACCTCTGAGCCCAAGCAAAGCCATCATAT
CCCCTGTGACCTGCATGTATACATCCAGATGGCCTGAAGCAAGTGAAGAATCACAAAAGAAGTGAA
AAGGGCCGGTTCCTGCCTTAACTGATGACATTCCACCATTGTGATTTGTTCCTGCCCCACCTTAAC
TGAGCGATTAACCTGTGAACTTCCTTCTCCTGGCTCAGAAGCTTCCCCACTGAGCACCTTGTGACC
CCCGCCCCTGCCTGCCATAGAACAACCCCCTTTGATTGTAATTTTCCTTTACCTACCCAAATCCTA
TAAAACGGCCCCACCCCTATCTCCCTTCGCTGACACTCTCTTTGGACTCAGCCTGCCTGCACCTAG
GTGATTAAAAAGCTTTATTGCTCACGC
Homo sapiens HHLA3 3′-UTR
NM_001031693.2
(SEQ ID NO: 94)
AAAGGGCCGGTTCCTGCCTTAACTGATGACATTCCACCATTGTGATTTGTTCCTGCCCCACCTTAA
CTGAGCGATTAACCTGTGAACTTCCTTCTCCTGGCTCAGAAGCTTCCCCACTGAGCACCTTGTGAC
CCCCGCCCCTGCCTGCCATAGAACAACCCCCTTTGATTGTAATTTTCCTTTACCTACCCAAATCCT
ATAAAACGGCCCCACCCCTATCTCCCTTCGCTGACACTCTCTTTGGACTCAGCCTGCCTGCACCTA
GGTGATTAAAAAGCTTTATTGCTCACGC
Homo sapiens ACAA2 3′-UTR
NM_006111.2
(SEQ ID NO: 95)
AGAGACCAGTGAGCTCACTGTGACCCATCCTTACTCTACTTGGCCAGGCCACAGTAAAACAAGTGA
CCTTCAGAGCAGCTGCCACAACTGGCCATGCCCTGCCATTGAAACAGTGATTAAGTTTGATCAAGC
CATGGTGACACAAAAATGCATTGATCATGAATAGGAGCCCATGCTAGAAGTACATTCTCTCAGATT
TGAACCAGTGAAATATGATGTATTTCTGAGCTAAAACTCAACTATAGAAGACATTAAAAGAAATCG
TATTCTTGCCAAGTAACCACCACTTCTGCCTTAGATAATATGATTATAAGGAAATCAAATAAATGT
TGCCTTAACTTC
Homo sapiens GSTM4 3′-UTR
GSTM4-001 ENST00000369836
(SEQ ID NO: 96)
TGCCTTGAAGGCCAGGAGGTGGGAGTGAGGAGCCCATACTCAGCCTGCTGCCCAGGCTGTGCAGCG
CAGCTGGACTCTGCATCCCAGCACCTGCCTCCTCGTTCCTTTCTCCTGTTTATTCCCATCTTTACC
CCCAAGACTTTATTGGGCCTCTTCACTTCCCCTAAACCCCTGTCCCATGCAGGCCCTTTGAAGCCT
CAGCTACCCACTTTCCTTCATGAACATCCCCCTCCCAACACTACCCTTCCCTGCACTAAAGCCAGC
CTGACCTTCCTTCCTGTTAGTGGTTGTATCTGCTTTGAAGGGCCTACCTGGCCCCTCGCCTGTGGA
GCTCAGCCCTGAGCTGTCCCCGTGTTGCATGACAGCATTGACTGGTTTACAGGCCCTGCTCCTGCA
GCATGGCCCCTGCCTTAGGCCTACCTGATCAAAATAAAGCCTCAGCCACA
Homo sapiens GSTM4 3′-UTR
GSTM4-003 ENST00000326729
(SEQ ID NO: 97)
TGGTCAATTTTCTGCATCAACTTGACTGGGCTAAGGGATGCTCAGATGGCAGGTAAAATCATTGTG
CTTGTGAGGGTGTTTCCAGAAGAGATTTGCCTTTGAATCAGAAGACAGCAAAGATTTCCTTCAGCA
ATGAAGGAGGCATCCACCAAACTGTCAGGGCCCAGAGAGAAGAAAAAGACAGGAAGGGTGAATTTG
ACCTCTCTGACTGGGACATCCATCTCTGCCTATCCTGGGACCTCCACACTCCTGGTTCTCTGGCCT
TCAGACTTGATCAGGGACTAACACCATCGCCTCCCACCCCCACCTTTGTTCTGAGGCCTTTAGCCT
CTGAATGATACCACTGGCTTTCCTGCTTCTCTATCCTGCAGTCGGCAGATCATGGGACTTCTTCAC
TCCAAAATTGTGTGAGCCAATTCCCATAACAGATAGATAAATTTATAAATAAACACACAAATTTCC
TAC
Homo sapiens ALG8 3′-UTR
NM_001007027.2
(SEQ ID NO: 98)
CTGAAACCTCCGCCTCCCAGAAAAGAAAAACCTCTTTTTAATTGGATGGAAACTTTCTACCTGCTT
GGCCTGGGGCCTCTGGAAGTCTGCTGTGAATTTGTATTCCCTTTCACCTCCTGGAAGGTGAAGTAC
CCCTTCATCCCTTTGTTACTAACCTCAGTGTATTGTGCAGTAGGCATCACATATGCTTGGTTCAAA
CTGTATGTTTCAGTATTGATTGACTCTGCTATTGGCAAGACAAAGAAACAATGAATAAAGGAACTG
CTTAGATATG
Homo sapiens C11orf74 3′UTR
(SEQ ID NO: 99)
TTCACAGAGGCATTTTGTGTGTGTGTGCTTATTTTAATTTTGTTCTTATTCTAGCAACATTAGAAT
AAAAGATAAACCTACTATAATTCCCTTTGTGGAAATTTAAAAAAAAAAAAAAAAAA
Mus musculus Ndufa1 3′-UTR
Ndufa1-001 ENSMUST00000016571
(SEQ ID NO: 100)
GGAAGCATTTTCCTGGCTGATTAAAAGAAATTACTCAGCTATGGTCATCTGTTCCTGTTAGAAGGC
TATGCAGCATATTATATACTATGCGCATGTTATGAAATGCATAATAAAAAATTTTAAAAAATCTAA
A
Mus musculus Atp5e 3′-UTR
NM_025983
(SEQ ID NO: 101)
CTGAATCTGAAGCCTGAAGTGCTGAGTCTTGAAGGTGAAGCATGTGGGCCCCTGTTCTGGCAGATG
GAAATCAACCTCACCTCCTGGGGGACAGGCTGCCCATCTCGTTGATAAATTGACTATGCCAATAAA
TTAACATGGTTCACTTTCAAAAA
Mus musculus Gstm5 3′-UTR
NM_010360
(SEQ ID NO: 102)
GCCAGAGCTCGCTGCTGCTGAGCCATCTTGCCCTGAGGGGCCCACACTCTTAGCTCACTGTCAGTC
TTGTTCCATCCTGTCCTGAGGGCCCCCACTCTGTCTCCTCTGCTCTTTCTAATAAACAGCAGTTGC
ATTA
Mus musculus Uqcr11 3′-UTR
NM_025650
(SEQ ID NO: 103)
GCAGCCCCTCCCCCACCACAGGCCTCGATGGTACCATGTGCCGAGGCCTCAGACACAGCGTAGTCC
TGTGGAAGACACTGAGGAAGCTGGACACTGGAGAGGTCTGCACCGCTCAGGGAGCTTCCATGTTGA
CAGACACTAGGGCTGCCTTGATGGGTGCAGCATTAAACCTTATTCTTATGCCTTGGA
Mus musculus IFi2712a 3′-UTR
IFi2712a-001 ENSMUST00000055071; NM_029803
(SEQ ID NO: 104)
GCTTAGGAGATGACACTTCTATCAGCTCAACTCAAAGCCTGTACAGACTACGCAGGAGATGAAGTT
CCAAAAGGCACCTTCAGAACCCTCACTGATGTCAAAGAATGATGAAAACAACAAAGTATATGGGCT
GGTGTTCCTAA
Mus musculus Cbr2 3′-UTR
NM_007621
(SEQ ID NO: 105)
TCTGCTCAGTTGCCGCGGACATCTGAGTGGCCTTCTTAGCCCCACCCTCAGCCAAAGCATTTACTG
ATCTCGTGACTCCGCCCTCATGCTACAGCCACGCCCACCACGCAGCTCACAGTTCCACCCCCATGT
TACTGTCGATCCCACAACCACTCCAGGCGCAGACCTTGTTCTCTTTGTCCACTTTGTTGGGCTCAT
TTGCCTAAATAAACGGGCCACCGCGTTACCTTTAACTAT
Mus musculus Atp51 3′-UTR
Atp51-201 ENSMUST00000043675
(SEQ ID NO: 106)
AGACCAATCTTTAACTTCTGATTTGAGTTCTTATTTGAATGTTCTTGGACCATGTGTAACAGGACT
GCTATCTGAATAAAATACTAGGTGTTGAAAACACTGCTGTGTTTTCTCTGTC
Mus musculus Tmsb10 3′-UTR
NM_025284
(SEQ ID NO: 107)
AAGCCTAGGAAGATTTCCCCACCCCACCCCACCCCGCCCCATCATCTCCAAGACCCCCTCGTGATG
TGGAGGAAGAGCCACCTGCAAGATGGACGCGAGCCACAAGCTGCACTGTGAAACCCGGGCACTCCG
AGCCGATGCCACCGGCCCGCGGGTCTCTGAAGGGGACCCCTCCACTAATCGGACTGCCAAATTTCA
CCGGTTTGCCCTGGGATATTATAGAAAATTATTTGTATGATTGATGAAAATAAAAACACCTCGTGG
CATGGTT
Mus musculus Nenf 3′-UTR
NM_025424
(SEQ ID NO: 108)
TGTCTAGCTGAGAAGCAGCCGGTTCTAGGGAGAAGTGAGGGGACAGGAGTTAAGTGTCCCTCGGAA
CAAGCGGAGGAAGCCTCCGAGTGCCCTGCAGCTGAATAAAGCGAATGTTT
Mus musculus Atp5k 3′-UTR
NM_007507
(SEQ ID NO: 109)
GGCGTCAGCGAGCTTGCTTTTCTCTAGTCGTTGAGAACGAATAAAGCTTCATTGTGTGAAAAAAAA
AAAAAAAAAAAAAAAAAAAAAAAAAAAAAAAAAAAAAAAAAAAAAAAAAAAAAAAAA
Mus musculus 1110008P14Rik 3′-UTR
1110008P14Rik-001 ENSMUST00000048792
(SEQ ID NO: 110)
GTGCCGGGAGCCCCCATCCAGGCCCTACCCTCACCTCTCTAGGCCATGTTCTGGCCTGGGTAGATA
CTACTTGGCTTAGACACCATCTCGGGTACTGGCCTCCAGATCCTAGTGGGTCTACCAGCCTGGACC
AGTCCCCATTCACTGCCCATCACCCTTCCTGGAGTCAGGTGCAATCCTACAGTTCTCCCACTTGTC
TGTCTTCTTTCCCCTCCATCCAGACTGAGAGTCCGAATTAAAGATGTCTCCCACACCACTGC
Mus musculus Cox4i1 3′-UTR
NM_009941
(SEQ ID NO: 111)
GAGCCCGCTGCCTGCCGGCTCCCTGCCTCCCTCACTCCCTCGGCATGCTGGAAGCTGCCGTATCCA
ATGGTCCATGCTAATAAAAGACCAGTTTACGTGGTG
Mus musculus Cox6a1 3′-UTR
NM_007748
(SEQ ID NO: 112)
AGAGAACCTGGCCTCCCCCAGGCAACAAAGGGACCACAGCACTGGTTTTGGACCCTTACTCTGTGT
GGACCACGAAAACCCTTTGGATGCTAAGCTCGTGTCTCCTTTCCTCAGATGGCGACCATTACTCTG
ATCTTCCATCCCTTCTGCTTGTAAGAGGAGATGCCTTAAATAAATAACTTAAACTCA
Mus musculus Ndufs6 3′-UTR
NM_010888
(SEQ ID NO: 113)
TGTGGGCTGTGTCCTGGTCCTCTGACTCCTATGGAACATCTCCACGCTGGGTGTTCTGTGTGAGGC
CACTGCTCTGTGAATGGTGTCCCTTGTTTTGAATAAAGGATGCTCCCACCATGAAAAAAAAAAAAA
AAAAAAA
Mus musculus Sec61b 3′-UTR
NM_024171
(SEQ ID NO: 114)
ATTGGGCTACATCCATCTGTCATCTGAAGAAGAAGAAGAAGGAAAAAAACCCAACATATCTTGGAC
CAAAAGTGTAGTGATTTTCTGTTCACGTGTATTATTTTACAGAGAATAAGAATTGACTTTGAGAAA
TCAGTTTTTTCTATGGCTAATAAACTTTGGAATTGCTTT
Mus musculus Romo1 3′-UTR
NM_025946
(SEQ ID NO: 115)
TTAGGGCTAGGATGCCCTGCAATACCTAAACTTCCCCATCCATTTCGACCCTTGTACAATAATAAA
GTTGTTTTCTTCTCGTTAAAAAAAAAAAAAAAAAA
Mus musculus Gnas 3′-UTR
NM_010309
(SEQ ID NO: 116)
GAAGGGAACACCCAAATTTAATTCAGCCTTAAGCACAATTAATTAAGAGTGAAACGTAATTGTACA
AGCAGTTGGTCACCCACCATAGGGCATGATCAACACCGCAACCTTTCCTTTTTCCCCCAGTGATTC
TGAAAAACCCCTCTTCCCTTCAGCTTGCTTAGATGTTCCAAATTTAGTAAGCTTAAGGCGGCCTAC
AGAAGAAAAAGAAAAAAAAGGCCACAAAAGTTCCCTCTCACTTTCAGTAAATAAAATAAAAGCAGC
AACAGAAATAAAGAAATAAATGAAATTCAAAATGAAATAAATATTGTGTTGTGCAGCATTAAAAAA
TCAATAAAAATTAAAAATGAGCAAAAAAAAAAAAAAAAAA
Mus musculus Snrpd2 3′-UTR
NM_026943
(SEQ ID NO: 117)
AGCCTGCTCCCTGCCCTGCGAAGGCCTGCAGAACCCTGCCCAGTGGGCGAGAAATAAAACCCTGTG
CTTTTTGGTTAAAAAAAAAAAAAAAAAAAA
Mus musculus Mgst3 3′-UTR
NM_025569
(SEQ ID NO: 118)
GGTGTGGAGGGCCTTCCGACTCTCACTCACCTCCAGCGACTCACCCTGATTTCCAGTTGCACTGGT
TTTTTTTTTTTTTTTAATATAATAAAAACTTATCTGGCATCAGCCTCATACCT
Mus musculus Aldh2 3′-UTR
NM_009656
(SEQ ID NO: 119)
AGCGGCATGCCTGCTTCCTCAGCCCGCACCCGAAAACCCAACAAGATATACTGAGAAAAACCGCCA
CACACACTGCGCCTCCAAAGAGAAACCCCTTCACCAAAGTGTCTTGGGTCAAGAAAGAATTTTATA
AACAGGGCGGGGCTGGTGGGGGGGAAAGCTCCTGATAAACTGGGTAGGGGATGAAGCTCAATGCAG
ACCGATCACGCGTCCAGATGTGCAGGATGCTGCCTTCAACCTGCAGTCCCTAAGCAGCAAATGAGC
AATAAAAATCAGCAGATCAAAGCCACGGGGTCAGTTCTCT
Mus musculus Mp68 (2010107E04Rik) 3′-UTR
NM_027360
(SEQ ID NO: 120)
CTGCTCCGAATCCACAAGATGAAGACGTCGGCTAAACTTGAGCAAGCTTTGTTAGATGGGAACATG
GAACATCACTGTACACTTATCTAAGTACCATTTATAATGTGGCATTAATAAATGTATCTGTGAATA
CC
Mus musculus Ssr4 3′-UTR
NM_001166480
(SEQ ID NO: 121)
GGGCAGCAACTTCAGCCGTCCATTGCTTCTTTCAATAAACAGTCACTATTTGACATGAGTACATTC
AAGAAAAAAAAAAAAAAAAAA
Mus musculus My16 3′-UTR
NM_010860
(SEQ ID NO: 122)
GGACATTCTGTATCCCGAGTCTGTTCCTTGCCCAGTGTGATTTCTGTGTGGCTCCAGAGGCTCCCC
TGTCACAGCACCTTGCCCATTTGGTTTCTTTTGGATGATGTTTGCCTTCCCCAAATAAAATTTGCT
CTCTTTGCCCTCCAAAAAAAAAAAAAAAAAAAAAAAAAAAAAAAAAAAAAAAAA
Mus musculus Prdx4 3′-UTR
Prdx4-001, NM_016764
(SEQ ID NO: 123)
AAAGTACTTCAGTTATGATGTTTGGACCTTCTCAATAAAGGTCATTGTGTTATTACCA
Mus musculus Ubl5 3′-UTR
NM_025401
(SEQ ID NO: 124)
AGGGGGATTCCTTCTCCTCCTCGCCCTGCTCTGCCCTGCCCTCCTCTCCCATCCTCATCTGACACT
GGTGTAGATGGTCATTTTTAACAGTTCACATGAATAAAAACTTGGCTGCTGCTTTGCTGCTGTC
Mus musculus 1110001J03Rik 3′-UTR
NM_025363
(SEQ ID NO: 125)
TGCAGAGAGTCCTCAGATGTTCCTTCATTCAAGAGTTTAACCATTTCTAACAATATGTAGTTATCA
TTAAATCTTTTTTAAAGTGTG
Mus musculus Ndufa13 3′-UTR
Ndufa13-201 ENSMUST00000110167
(SEQ ID NO: 126)
GGCCTGAGCCAACGCACATAATAAAGAGTGGTC
Mus musculus Ndufa3 3′-UTR
NM_025348
(SEQ ID NO: 127)
ATGCCTCTGCTGATGGAAGAGGCCCCTTCCCTGTTGCTCTCCAATAAAAATGTGAAAACTAATAAC
CCC
Mus musculus Gstp2 3′-UTR
NM_181796
(SEQ ID NO: 128)
TGGACTGAAGAGACAAGAGCTTCTTGTCCCCGTTTTCCCAGCACTAATAAAGTTTGTAAGACAAAA
AAAAAAAAAAAAAAAAAAAAAAAAAAAAAA
Mus musculus Tmem160 3′-UTR
NM_026938
(SEQ ID NO: 129)
ACAACAGGGCTGTGGGGACTGGCTGGGCCTGACGACTGGGACATTAAAACCTGACCCTTCCGCAAA
AAAAAAAAAAAAAAAAAAAAAAA
Mus musculus Ergic3 3′-UTR
NM_025516
(SEQ ID NO: 130)
CTCTCTCCCTTCCCCACAGCTTGTCCTGCCCTCTCTTCCCCTGTGGGTTTACCCTCCAGCCTGTCA
ACTACCCATATCCTCTCCTCAGCCAGCCCAGCCCAGGGCAATAAATATGAATTGTGATAGGAA
Mus musculus Pgcp 3′-UTR
NM_018755
(SEQ ID NO: 131)
GGAGAACAAGAAGAGAGGACCTTGTTCTCTGTAGTTGGGAATCCCAACTCTGAATCTTTACAACAT
CCATCGTCACAAAAGAGTGTTATACATTTAATCCACAGGGCATAGTTTTCTTTATACCTTCTGTTA
ATCATCTTTCCTTAATACTTTCTTATCTGTTTCTAGAATAAATCATGATCCCTACTGCACCACCTT
GAAAATGTTGTTTCCAGTTTTAAAATAAGCAATAAATATTTGAAATGCTTCTGATTTTTCATTTTC
ATTTAAAAACATTAAATTAAATGTAATGAGA
Mus musculus Slpi 3′-UTR
NM_011414
(SEQ ID NO: 132)
GCCTGATCCCTGACATTGGCGCCGGCTCTGGACTCGTGCTCGGTGTGCTCTGGAAACTACTTCCCT
GCTCCCAGGCGTCCCTGCTCCGGGTTCCATGGCTCCCGGCTCCCTGTATCCCAGGCTTGGATCCTG
TGGACCAGGGTTACTGTTTTACCACTAACATCTCCTTTTGGCTCAGCATTCACCGATCTTTAGGGA
AATGCTGTTGGAGAGCAAATAAATAAACGCATTCATTTCTCTATGCAAAAAAAAAAAAAAAAAAA
Mus musculus Myeov2 3′-UTR
NM_001163425
(SEQ ID NO: 133)
GGCCGCCCGGTCCTATGTGCTCCATGTCTGTGATGTGTCTGGAGTCTCTCGGGACACGACCAGCTG
ATTGTAGACACCGTGTTGATATCACTAGAAATGAAGACCTTGTCAACCAATAGAGGAACTGTCTGA
ACCAACTGGGTACTGATGTCTCTGGGAATGCCAGCCCGTGTCCTTGTTTAAGTTAATAAAGAACAC
TGTAACACGCAGGGTGATTTTAAAAAAAAAAAAAAAAAAAAAA
Mus musculus Ndufa4 3′-UTR
NM_010886
(SEQ ID NO: 134)
ACTATGAAGTTCACTGTAAAGCTGCTGATAATGAAGGTCTTTCAGAAGCCATCCGCACAATTTTCC
ACTTAAGCAGGAAATATGTCTCTGAATGCATGAAATCATGTTGATTTTTTTTTTTTTTGGAGTTTA
TTACACTGATGAATAAATCTCTGAAACTTG
Mus musculus Ndufs5 3′-UTR
NM_001030274
(SEQ ID NO: 135)
GCGGGGCAGCTGGAGGCCGCTGTCATGCTCTGTTTTCCCCTGGAGAGAATATTTAAGGAAAGCTCC
TTCATTAAGTATTAAGTATGTGGAAATAAAGAATTACTCAGTCTTAAAAAAAAAAAAAAAAAAAAA
AAAAAAAAAAA
Mus musculus Gstm1 3′-UTR
NM_010358
(SEQ ID NO: 136)
GCCCTTGCTACACGGGCACTCACTAGGAGGACCTGTCCACACTGGGGATCCTGCAGGCCCTGGGTG
GGGACAGCACCCTGGCCTTCTGCACTGTGGCTCCTGGTTCTCTCTCCTTCCCGCTCCCTTCTGCAG
CTTGGTCAGCCCCATCTCCTCACCCTCTTCCCAGTCAAGTCCACACAGCCTTCATTCTCCCCAGTT
TCTTTCACATGGCCCCTTCTTCATTGGCTCCCTGACCCAACCTCACAGCCCGTTTCTGCGAACTGA
GGTCTGTCCTGAACTCACGCTTCCTAGAATTACCCCGATGGTCAACACTATCTTAGTGCTAGCCCT
CCCTAGAGTTACCCCGAAGGTCAATACTTGAGTGCCAGCCTGTTCCTGGTGGAGTAGCCTCCCCAG
GTCTGTCTCGTCTACAATAAAGTCTGAAACACACTTGCCATGAAAAAAAAAAAAAAAAA
Mus musculus 1810027O10Rik 3′-UTR
1810027010Rik-001 ENSMUST00000094065
(SEQ ID NO: 137)
AGTCTCTTGTTTAAGCGCCCAGTCCTGGCCTTTCTGGGTAATTGGGCGCAGAGGGAAGGAGCCAAT
GTTGAAGCAGAAAAGAAATTAAAAGAAAAAGGCATATAAAGAA
Mus musculus 1810027O10Rik 3′-UTR
BC117077
(SEQ ID NO: 138)
AGTCTCTTGTTTAAGCGCCCAGTCCTGGCCTTTCTGGGTAATTGGGCGC
Mus musculus Atp5o 3′-UTR
NM_138597
(SEQ ID NO: 139)
GAGACTGTCACCTGTGTGAGCTCTTGTCCTTGGAGCAACAATAAAATGCTTCCTG
Mus musculus Shfm1 3′-UTR
NM_009169
(SEQ ID NO: 140)
CATCTGGGAATGTCCCAGGAACCTCAATCATGGACTCTACCACAGTCTAGGACAGAGAAAGCAGGA
CGGGATACTTTAAAGAACATGTTTATTTCATTATCTGCTTCAATTTATTTTTGTTTTATAACAAAA
AAAATAAGTAAATAAATGTTTTGATTTAATCTTTTTGGTTCA
Mus musculus Tspo 3′-UTR
NM_009775
(SEQ ID NO: 141)
AGGCACCCAGCCATCAGGAATGCAGCCCTGCCAGCCAGGCACCATGGGTGGCAGCCATCATGCTTT
TATGACCATTGGGCCTGCTGGTCTACCTGGTCTTAGCCCAGGAAGCCACCAGGTAGGTTAGGGTGG
TCAGTGCCGAGTCTCCTGCAGACACAGTTATACCTGCCTTTCTGCACTGCTCCAGGCATGCCCTTA
GAGCATGGTGTTTTAAAGCTAAATAAAGTCTCTAACTTCATGTGTAAAAAAAAAAAAAAAAA
Mus musculus S100a6 3′-UTR
NM_011313
(SEQ ID NO: 142)
AATGGGACCGTTGAGATGACTTCCGGGGGCCTCTCTCGGTCAAATCCAGTGGTGGGTAGTTATACA
ATAAATATTTCGTTTTTGTTATGCCT
Mus musculus Taldo1 3′-UTR
NM_011528
(SEQ ID NO: 143)
TGCAACACCCGAGGCCCCAGTCCTGCACCGAGGCTGACCCCAGACCTGCACTGCCTTTGAGCTGGG
TCCTAATTGCACATGGCTTGTGACGAATGAATCTTGCATTTTTTAGTGATCGGAGAAGGGATGGAT
CATAGGATTCTGATTTTATGTGAAATTTTGTCTAATTCATTAAAGCAGTTGCTTTTCCTATGCTGT
TT
Mus musculus Bloc1s13′ -UTR
NM_015740
(SEQ ID NO: 144)
ACTAAAACCCACCCCTCTTACTTCACCCTCCTGGACAGGAGGGAAACTGGTGAGCCACGAATAAAA
ACACAAGCTTCCATTCT
Mus musculus Ndufb11 3′-UTR
NM_019435
(SEQ ID NO: 145)
TGGCTTACCGAGCAGGGCCTAAGAAGCATTACTCATCCGCTGCTTGTTATTTACCTGGTTCCTCAG
AACACCTTATTAAAGGAATTGAAAGTA
Mus musculus Map1lc3a 3′-UTR
NM_025735
(SEQ ID NO: 146)
GTCAAGAGGAGGGGAGGGGGGTGGCTGGGAGTTCTGGTCAGGTTCTCCCCAGGGAGGTCCTGGCTC
CTAAACTAAGCTATTTCAGTCCCCAGTGGATTAGGCAGAGATGTGACACCCACTCCCCCCCCCAGG
TAGGGGCCACCAGCCAGCCTACCACATCCTGGGTAGGTCCTGGGCCAGTCATGTTCGGGTTGCTCT
TTTGGGTGCTGGCTGGGTTGGGAGTGGGTGGGGAGCAGCATCCCTGCTCTGTGGGGTTTGTCATTT
TGTTAGGCCCTTGCCTGTCTGCCCATCTTGCCCCTCATCCACCTGAGGCTTTGCCTCCTGCCAGGA
CCTGCCCCACCCCTGAAAGGCTGGCTCCCCTTGTCCTGACTCGGTGTATGGATCTGTGGTCATTTC
CTCTGCAGAAAGAATAAAGACTGCTCAGGCCTGCCTGGCCAAAAAAAAAAAAAAAAAA
Mus musculus Morn2 3′-UTR
NM_194269
(SEQ ID NO: 147)
ACCTGCTGCCTTAACGCTGAGATGTGGCCTCTGCAACCCCCCTTAGGCAAAGCAACTGAACCTTCT
GCTAAAGTGACCTGCCCTCTTCCGTAAGTCCAATAAAGTTGTCATGCACCCACAAAAAAAAAAAAA
AAA
Mus musculus Gpx4 3′-UTR
NM_008162.2
(SEQ ID NO: 148)
CTAGCCCTACAAGTGTGTGCCCCTACACCGAGCCCCCCTGCCCTGTGACCCCTGGAGCCTTCCACC
CCGGCACTCATGAAGGTCTGCCTGAAAACCAGCCTGCTGGTGGGGCAGTCCTGAGGACCTGGCGTG
CATCCCTGCCGGAGGAAGGTCCAGAGGCCTGTGGCCCTGGGCTCGAGCTTCACCCTGGCTGCCTTG
TGGGAATAAAATGTAGAAATGTGAAAAAAAAAAAAAAAAA
Mus musculus Mif 3′-UTR
NM_010798.2
(SEQ ID NO: 149)
GTCCTGGCCCCACTTACCTGCACCGCTGTTCTTTGAGCCTCGCTCCACGTAGTGTTCTGTGTTTAT
CCACCGGTAGCGATGCCCACCTTCCAGCCGGGAGAAATAAATGGTTTATAAGAGACCA
Mus musculus Cox6b1 3′-UTR
NM_025628
(SEQ ID NO: 150)
CCTGGCTCCGCCCACCTCTCCTCTGTTCTTTGTCTTTCTCCCCGGATAGAAAAGGGGGACCTCAGC
ATATGATGGTCCTTACCCTGGGACCCTGAATCATGATGCAACTACTAATAAAAACTCACTGGAAAA
GTT
Mus musculus RIKEN cDNA2900010J23 (Swi5) 3′-UTR
NM_175190
(SEQ ID NO: 151)
GCAGCTTCTTGGAGATTTTCATCTACAGCCCACAGGGACAGGAGGATGGGGGCATAAAAGGCAGAG
TCTAGACAGTATGTTCATATGGTTTTCAGATTTTAAAAGATGCTAGAAGCCCTCCAAAGTTTGGGG
TGGGTTCTAGAGAAGAGGAGTATTGGGAGGGGTGGGTATTGTCAATGTTAAGGTTCCTAAACATAC
TTGTGAGTAGGTGTGTGTGGTTGTCCCTTTTGTTAATAAACATATGAGCAGTCAAAAAAAAAAAAA
AAA
Mus musculus Sec61g 3′-UTR
NM_011343.3
(SEQ ID NO: 152)
GTCCTTCTCATCATGGGACGAGTGAGCCAGAGCGGGGGAAAGGGCATGAAGTAAAGCGTTGCCTGA
ATGCTGTGTGGTGTTTTGTTTCTTCCTCCTTCCTATGAGGTTTTCTACTTCTCAATTAAAATAATT
TCAAAATAAACACTTTTTCCATAACAGA
Mus musculus 2900010M23Rik 3′-UTR
BC_030629
(SEQ ID NO: 153)
CCGTGGGGTCTGATACTCATCAATAAAACTGCCTGGTTTCTCCCACAAAAAAAAAAAAAAAA
Mus musculus Anapc5 3′-UTR
Anapc5-201 ENSMUST00000086216
(SEQ ID NO: 154)
CCAGGACTCCCTGCTTGATGGTGTGCATTTAGGGGTGGGTCATTACATGCTATCTTGTCAATAAAC
TGTTCTGATCAGTTTGTCTGAAGTGGGTTTTTTTTTATTTTTCTGGGTTGAATTGTCAGTATCTTT
GTTAAGAACTGTGTATCTAGGGGCTGGAGAGATGGCTTAGCAGTTAAGAGCACTAACTGTTCTTCT
AAAGGACCTGGGTTCAATTCCTAGCACCCTCATGACAGCTCACAGCTGTCTGTAACTCCTGTTCCA
GGGACTCTGACACCCTCAGGCAGACATAAAAGCAGTCAAAACACCGATGTACATAAAATTAAAATA
AATTATTT
Mus musculus Mars2 3′-UTR
BC132343.1
(SEQ ID NO: 155)
GAACTCAGCTCTTACTGACTGGTAGTAAAAGATCAAATGTATTCTTTTTGCGTTTTTAAGTAAAGT
CATGC
Mus musculus Phpt1 3′-UTR
NM_029293
(SEQ ID NO: 156)
AGCTCTGCCCCACCCCCCACCCCCCGGACTAAGTCAGGTCTCTGCTCTTGCTGTGTTCTGTTTTGA
GGGGCTGGCCCTGTGCTTTCCTTTTGTACCTTAGGCAGCATAGCACCTGCCAGGCCTTAGAGGCCA
GACCAATCTGGTCCATAGGAATTAAAAGCATTGATATGCCTACT
Mus musculus Ndufb8 3′-UTR
NM_026061
(SEQ ID NO: 157)
GGAGGCTTGATGGGCTTTTTGCCCTCGTTCCTAGAGGCTTAACCATAATAAAATCCCTAATAAAGC
Mus musculus Pfdn5 3′-UTR
NM_027044
(SEQ ID NO: 158)
GAGTGCACTGCAGAAATGAAGCAGAGTGAGGGACCCTTCTTCAAGGGGCCTGGGACTTTTTCCGGC
AATGGCCTCCTGGGAAAGTGGCCTGGGAAGAGAGTGTTTTGTGTTTAATGTTAATAAATGTGACCG
CTGCGCAAAAAAAAAAAAAAAAAAAAAAAAAAAAAAAA
Mus musculus Arpc3 3′-UTR
NM_019824
(SEQ ID NO: 159)
GAGGAGCCTGGGCAGCACCATCACGTGGAGACACATCATAGGACACACAGGCCAATGTGTCTGTTC
ATACCTACCGTATCAAGGAGAGAAGAGAGCCTGTCTTTGCTGGAAAAGCTCTTGGTCAAGAATTGG
GAGGGTGGGTGTTGGGCGATTTCGATTTTTGGCAGTTTTAAGCTGGTACTTAATATATAATAAATG
TCACTGCTTATGTTAGACATTGAATTAAAACATTTTTGAGAAAAAGCTTTAAAAAAAAAAAAAAAA
AAAAAAAAAAAAAA
Mus musculus Ndufb7 3′-UTR
NM_025843
(SEQ ID NO: 160)
GGATTACCCGCCAGCCTGTGGACCTATCAGTGAAATAAAAGCTTTGGGTCACCTGCCT
Mus musculus Atp5h 3′-UTR
NM_027862
(SEQ ID NO: 161)
AGCAGCCTGGGACGGAGCCCCGGCCGACATGAAATAAAACATTTAAATAGT
Mus musculus Mrp123 3′-UTR
NM_011288
(SEQ ID NO: 162)
CCTATGACAGCAGGATTTGGACCACAGACCCTAGTGAGCACAGTGGTTCTGACAAGCCCAAATAAA
AATTCTTTGTGGAG
Mus musculus Tomm6 3′-UTR
NM_025365.3
(SEQ ID NO: 163)
CCAGAGAATGGAACTCCTGTGTATTCAGACTTTCCAAAGACAGCCTACTGTCTGTGACCACAAGAT
CCTACCTGAGTGGCAGCTGAAGTTGACTCCCTCTCCTTGCCTGAACCCCCCCCCACTGCCCCCCCA
TCCCCCAGTGTCGGCTGAGATGTTGCCTCTGCACGGTTCTGTGTGCAGTTCCCAACTTTCTGCAGA
AGATGGTCCTTGCCCTTGTCCTGAAGAGTAGTAATGGTTCTTGAAAAAGATTTCAAATAAAGCCTG
CACATAAAAGACAGGTATTTTATTCTTTTAATAAGAAACTTATTACAAAAACAAGGTGTAAAAAGT
CCGCTTACAAAAATCAAATAAACATGACTTGTATTTCAAAAAAAAAAAAAAAAAAAA
Mus musculus Tomm6 3′-UTR
Tomm6-002 ENSMUST00000113301
(SEQ ID NO: 164)
CCAGGTGAGAGCAGTTCTCCTGTGTTTCCCCGTTTCTGATGCTGTTATCTGCTTACAGAGAATGGA
ACTCCTGTGTATTCAGACTTTCCAAAGACAGCCTACTGTCTGTGACCACAAGATCCTACCTGAGTG
GCAGCTGAAGTTGACTCCCTCTCCTTGCCTGAACCCCCCCCCACTGCCCCCCCATCCCCCAGTGTC
GGCTGAGATGTTGCCTCTGCACGGTTCTGTGTGCAGTTCCCAACTTTCTGCAGAAGATGGTCCTTG
CCCTTGTCCTGAAGAGTAGTAATGGTTCTTGAAAAAGATTTCAAATAAAGCCTGCACATAAAA
Mus musculus Tomm6 3′-UTR
(SEQ ID NO: 165)
CCAGAGAATGGAACTCCTGTGTATTCAGACTTTCCAAAGACAGCCTACTGTCTGTGACCACAAGAT
CCTACCTGAGTGGCAGCTGAAGTTGACTCCCTCTCCTTGCCTGAACCCCCCCCCACTGCCCCCCCA
TCCCCCAGTGTCGGCTGAGATGTTGCCTCTGCACGGTTCTGTGTGCAGTTCCCAACTTTCTGCAGA
AGATGGTCCTTGCCCTTGTCCTGAAGAGTAGTAATGGTTCTTGAAAAAGATTTCAAATAAAGCCTG
CACATAAAA
Mus musculus Mtch1 3′-UTR
NM_019880
(SEQ ID NO: 166)
CCTAAGCTGCCCGACCAAACATTTATGGGGTCTTAGCCTACCCCTGGTGAGGACCCATCATCTCAG
ATGCCCAAGGGTGACTCCAGCCCAGCCTGGCTTCATGTCCATATTTGCCATGTGTCTGTCCAGATG
TGGGCTGGTGGAGGTGGGTCACCTGGGACCTGGGGAAGCCTGGGGGAGCAGTGTTGGGGTGGCATC
CCCTTCCTGCCTAGAGGTACTGGAGTCCATCTTGTACTCAGGCAGAGGCAGGCTGCAGAGGCAAAC
GTCACTCAGTGGCAAGGCTTCCCTGCACCTCTAGCCCAGCTCATCCTGCCAGTCAGCCAGAAGCAC
CCCCGCCCCCCACTTCCTGCTTTGTAAATTGGGCGCCATCACACCTGGGCCATGGGAGGCTGGAGC
TATGTTCCCAACACTAATTTTCTTATACAAGGGTGGTGCCTTCTCCTGAATAGGAAATCATGTTCT
CCTCAGACCATCCCCTCATCTGCTTGTCTGTGCTGGTGACGCCAGGTGTGAGGGTTCAGTCACTGT
GCTGGGTGCGAATACGCACAGGTTACATAGGCCGACATCTAGTCCTCCCCTCGTGGTAAGATAGAC
CCATCTCCTCGAATAAATGTATTGGTGGTGATTTGGA
Mus musculus Pcbd2 3′-UTR
NM_028281
(SEQ ID NO: 167)
TCTGCGCCTGCCTTGTCTGCAGCGTTGTTTGCAAGCCACTTATGTTAATAAATTGTCATAAAGTAG
TTCATAGTTACATGTATACATTGTTGTATGATTGATGCTCAAATACAGAATGATTTGAAGCCAAAA
AAAAAAAAAAAAAAAAAAAAAAAAAA
Mus musculus Ecm1 3′-UTR
NM_007899
(SEQ ID NO: 168)
GTCACCCTGAGCCTCAGAGGATTAGATGGGGGAACTCCGCCCTACTCCACCCTCCTCGAACACTCA
TTACAATAAATGCCTCTTGGATTTGGC
Mus musculus Hrsp12 3′-UTR
Hrsp12-001 ENSMUST00000022946
(SEQ ID NO: 169)
CTATAAGTAGCCATGCTGATGTTGACTCCGGAGGTTTTAGAATGTCTTTCACACTTTAATTTTTAC
AAATGATGCTGGGAAGTATAAAAATGACCAGAGTGGTTGAAGTTATTGTGGAAGTGATCAAATATG
TGGAGATTTGACATTAATTGGAGATTATTCAGTATAGTGACTGATGTTCTAATTTCACTTATGTTG
CTGGGTGTGAGAGAAGAGGTGCACAGCTACTGAGATGGGAAGCAGAAGGAAAGATGGGCTGTTGTA
CATGAGAAATAGTAAGGAGCACATCTACTTAAATCATATTAATTTGCTCATGTGAAATACTTAGTT
CTTATGTTAGATATAAGAAACTAAATTGAAATATTCAAACTTGAATAGTACCAGGAGAACAAGTGG
ACCAAAATCTTATACAGATAATATTACTTTAATTGAAATAAAAAATAGATGTGTAACTTTCC
Mus musculus Mecr 3′-UTR
NM_025297
(SEQ ID NO: 170)
TTGCTCCAGAGGACCAGGAGGAAAGCAGGAGAGGCAAGACTGGCTGTCTGCTGGCCCCTCCATGAG
AACCCCAGCCTTCCCAGACTGCCTCACCCATATTGTCTCTTCCTACCAGGAGGGTGGGGGACCAAC
TCTAGGCTCCCTAATAAACCCTTAACTTCCCGAGTGGAGGATGAAGAGTAC
Mus musculus Uqcrq 3′-UTR
NM_025352
(SEQ ID NO: 171)
ACGGCCTGCACCTGGGTGACAGTCCCCTGCCTCTGAAAGACCCTTCTCTGGGAGAGGAATCCACAC
TGTAGTCTTGAAGACAATAAACTACTTATGGACTTCCCTTTGAAAAAAAAAAAA
Mus musculus Gstm3 3′-UTR
NM_010359
(SEQ ID NO: 172)
GCCCCTGCCATGCTGTCACTCAGAGTGGGGGACCTGTCCATACTGCGGATCCTGCAGGCTCTGGGT
GGGGACAGCACCCTGGCCTTCTGCACTGTGGCTCCCGGTTCTCTCTCCTTCCCGCTCCCTTCTGCA
GCTTGGTCAGCCCCATCTCCTCATCCTCACCCCAGTCAAGCCCATGCAGCCTTTATTCTCCCCATT
TTTTTTTCACATGGCCCCTTCTTCATTGGTGCCCAGACCCAACCTCACAGCCCTTTTCTGCAATCT
GAGGTCTGTCCTGAACTCAGGCTCCCTAGAGTTACCCCAATGGTCAACACTATCTTAGTGCCAGCC
CTCCCTAGAGATACCCTGATGGTCAATACTATCTTAGTGACGGCCCTCCCTAGAGTTACCCTGAAG
GTCAATACTCGAGTGCCAGCCTGTTCCTGTTTAAGGAGCTGCCCCAGGCCTGTCTCATGTACAATA
AAGCCTGAAACACACTTGAAACACAATAAACACTGAACACTTGCTGTGA
Mus musculus Lsm4 3′-UTR
NM_015816
(SEQ ID NO: 173)
TCACTCCCTGCCTGAGCCGAGCCCAGAACGGTGGGTGAGGCCTCAGGGCACCTTTGTGTGAAGCCC
CACTTGGCGTCTGGTCCAGTGAAGTCCCTCGCTGGCCACTGACTCAGTTTCTGGAAGGTTCCGAGT
CTGAGGTGCCTGTGGAGCCTTAGATGCCCTTTGAAGGGCTGACTTCTTCCAGGCATGTTTGAGTTT
CAGTTGGAGCTGCAGGCTCAGCCCATGGCGGCTCACCTGTCCTTTACCAGCCATACCCTGTACATC
TTCTGTTTGAAAAATAAAAGCAAACACCATAGAAAGAAAAAAAAAAAA
Mus musculus Park7 3′-UTR
NM_020569
(SEQ ID NO: 174)
AGCCCAAGCCCTGGGCCCCACGCTTGAGCAGGCATTGGAAGCCCACTGGTGTGTCCAGAGCCCAGG
GAACCTCAGCAGTAGTATGTGAAGCAGCCGCCACACGGGGCTCTCATCCCGGGTCTGTATGTTTCT
GAACCTTGCTAGTAGAATAAACAGTTTACCAAGCTCCTGCCAGCTAAAAAAAAAAAAAAAAAA
Mus musculus Usmg5 3′-UTR
NM_023211
(SEQ ID NO: 175)
ATGGATTTTGAAATGTCTGACCTCACCTGTTAAGTCCCATGCCTGAAGAAGCTGATGTGAACTCAT
CATGTAATACTCAATTTGTACAATAAATTATGAACCCAAAAAAAAAAAAAAAAAAAAAAAAAAAA
Mus musculus Cox8a 3′-UTR
NM_007750
(SEQ ID NO: 176)
AGGGAGCAGTCTTCCCTCATCCTTTGACTAGACCACTTTTGCCAGCCCACCTTGATCATGTTGCCT
GCATTCCTGGCTGGCCTTCCCCGGGATCATGTTATTCAATTCCAGTCACCTCTTCTGCAATCATGA
CCTCTCGATGTCTCCATGGTGACAACTGGGACCACATGTATTGGCTCTGCTTGGTGGGGTCCCCCT
TTGTAACAATAAAGTCTATTTAAACCTTGCTCC
Mus musculus Ly6c1 3′-UTR
NM_010741
(SEQ ID NO: 177)
TGGTCCTTCCAATGACCCCCACCCTTTTCCTTTTATCTTCATGTGCAACCACTCTTTCCTGGAGTC
CTCTAGTGACAAATTATATGTTATAGAAGGTCCAATGTGGGGATAGTGTGTGGAACACCCTGTTTC
ACCTTTATAGCCCCTGCTGGGTAAGTGCCCGACTCCTCTCTAGGGCTTTCAAATCTGTACTTCTTG
CAATGCCATTTAGTTGTGGATTTCTATTCTTGGCCCTGGAGGCATGTGGCCAGCACATGCAACAGG
CAGTATTCCAAGGTATTATAGTATCACCATCCACACATAAGTATCTGGGGTCCTGCAGGGTTCCCA
TGTATGCCTGTCAATGACCCCTGTTGAGTCCAATAAAAGCTTTGTTCTCCCAGCCAAAAAAAAAAA
AAAAAAA
Mus musculus Ly6c1 3′-UTR
NM_001252058.1
(SEQ ID NO: 178)
ACTCATAAAAATGCTCCTGCCTCGGTCTTCCAAGTTCTAGGATTGCAAGTCTGACTTCAACATGCC
TTACAGACAACTCTGGGACATCCAGGCCTAGTGGCATGTTGCCCAGATATGGGGATGCTCTGTGGC
CCCTGCATAAGAAGTGAGTCACTCCCTGATTTCTTGCAGACTCTCAAAGAAGGAAACTAAAGACCC
GTCAGTGCCTTTCTTTCTGCCCTGCTGGTGTGCCAATCAGGGATCCTAACATCAGGGAGAGGACTT
CCTGTTGCAGCGAAGACCTCTGCAATGCAGCAGTTCCCACTGCAG
Mus musculus Cox7b 3′-UTR
NM_025379
(SEQ ID NO: 179)
TCGTGCCAGCTGGTACAATAATCAAGGAATTGTTTAAAACCAACTTATAAGTGAATGCCAAGTCAA
AGAATCATGTACTCATTATACTATGGCAGATTGAAGAACAAATAAAGAAATAAAGTACCTTAACCT
TCATTCTAGGCTTTGTTTTTTTCCTTTGTAAATGAAGCCCAAGCATGGTGACTTCTCATTTATTTA
AGCTGTATTGTCTCTTAAAATGGCTTTTTACCCTATGAGGTGGTATGAGGGAAATCTATGATCAGG
AGGGCACCTTTATAGTAAGCTGAAATTACAGAGAATGAAGAAATAAGCACAGAGCTGTTTTAGGAG
CCCACTGGGTCATTGGCCATATAGGTTATGCTTACTGCCCTCTACCTCGTGGTTATATTTGGAATT
GCCATTAGCTCCCTTCTGCTTAGAGACTGGACTGTCACCAAACCCAAGGGGATAGTGATCCTGTAA
TGATCCTGTGTGAACTAGGTTTGCTAAAGACTACCACCTCCTTACACTGTATGGCATATTCATCTG
AAATAGGTGCTAATTTTTCAGCATAATCCTTAATCTTTAGGATCTGTCATACTTCCTAGTAATTTA
ACTGTTGCTGAAGAAATAAAGGCTATCTGTTAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
AAAAAAAAAA
Mus musculus Ppib 3′-UTR
NM_011149
(SEQ ID NO: 180)
AGAGCCTGGGGGACCTCATCCCTCTAAGCAGCTGTCTGTGTGGGTCCTGTCAATCCCCACACAGAC
GAAGGTAGCCAGTCACAAGGTTCTGTGCCACCCTGGCCCTAGTGCTTCCATCTGATGGGGTGACCA
CACCCCTCACATTCCACAGGCCTGATTTTTATAAAAAACTACCAATGCTGATCAATAAAGTGGGTT
TTTTTTATAGCTTGAAAAAAAAAAAAAAAAAAA
Mus musculus Bag1 3′-UTR
NM_009736
(SEQ ID NO: 181)
AGTGCAGTGGAGAGTGGCTGTACTGGCCTGAAGAGCAGCTTTACAGCCCTGCCCTCTCTGGAACAG
AAGTCGCCTGTTTCTCCATGGCTGCCAGGGGCAACTAGCCAAATGTCAATTTCCCTGCTCCTCCGT
CGGTTCTCAATGAAAAAGTCCTGTCTTTGCAACCTGAATTAGACTTGTGTTTTCTCAAAAAAAAAA
AAAAAAA
Mus musculus S100a4 3′-UTR
S100a4-201 ENSMUST00000001046
(SEQ ID NO: 182)
AGACTCCTCAGATGAAGTGTTGGGGTGTAGTTTGCCAGTGGGGGATCTTCCCTGTTGGCTGTGAGC
ATAGTGCCTTACTCTGGCTTCTTCGCACATGTGCACAGTGCTGAGCAAATTCAATAAAAGGTTTTG
AAACTATT
Mus musculus Bcap31 3′-UTR
NM_012060
(SEQ ID NO: 183)
AGGCTTGGTGTTTCCCTGCCTGCCGCTGGCTTCTACCTGACCCATGCTTACTGCTTCCTTGGAGCC
CAGACTATCCCTCTGGTACTTGGGTTTATTCCCTACTTCCCCAATTTTCTTCCATGGCTTATAGAT
CATTATTTTGGCACCATTACACATACTGCTCTTATACCAAAAGGGACCTGATTGTTGTTTATTCAG
AGTACTTTTGCCACTGTTCTGCCTGGCTAGGGCACTTTCCACTCCTGGAAGTGTAGAAAAGCACTG
GTGACCTGGCCTGCAGTTTGAACCCCTTTTTATTTTGCAATGTACCCTAAAGGAGGCTGCTGTGAA
GCAGGTCAACTGTTTTATCCTGAGGGGAATAAATGTTGTTATGT
Mus musculus Tecr 3′-UTR
NM_134118
(SEQ ID NO: 184)
GCAGCTCCTCACGGCTCTGCCCAGTAATACTCTCCACCCCTCACTGCCCCTGTCCTGATGTGTGGC
TGGCCATGGCTCTCCAGCAGCAACAATAAAACCTGCTTACCCAAAAAAAAAAAAAAAAAA
Mus musculus Rabac1 3′-UTR
NM_010261
(SEQ ID NO: 185)
AGTGTCCTCCAGGACCTGCCGGCCTCTCCTGCCGGCCGGCTGTCCCATCTCTGTCTGTTCTCGTCC
TACCTGGCCTTGCTGCTCAGCTCCGAGCCTTCCACCTGAGGCCTCAAACCCAGGGAGGGGCTTTTG
TCTTTGGAAATAAAGCTGTTACAATTGCTATTTGGCCAA
Mus musculus Robld3 3′-UTR
NM_031248 (Lamtor2)
(SEQ ID NO: 186)
CAGCGTGATGGAGGCTGGAGTAGAAAAGGGATGATGATCTGGAGGGAGGGGCGGGGCCCTAGAAAC
GCCATATCGGGCGAGGTACAGGAAGGGGGGGTTGCTTTTTTCTGAATAAATTTTCAACTCTTAAAA
AAAAAAAAAAAA
Mus musculus Sod1 3′-UTR
NM_011434
(SEQ ID NO: 187)
ACATTCCCTGTGTGGTCTGAGTCTCAGACTCATCTGCTACCCTCAAACCATTAAACTGTAATCTGA
AAAAAAAAAAAAAA
Mus musculus Nedd8 3′-UTR
NM_008683
(SEQ ID NO: 188)
AGAAACTTGGTTCCGTTTACCTCCTTGCCCTGCCAATCATAATGTGGCATCACATATCCTCTCACT
CTCTGGGACACCAGAGCCACTGCCCCCTCTCTTGGATGCCCAATCTTGTGTGTCTACTGGTGGGAG
AATGTGAGGACCCCAGGGTGCAGTGTTCCTGGCCCAGATGGCCCCTGCTGGCTATTGGGTTTTAGT
TTGCAGTCATGTGTGCTTCCCTGTCTTATGGCTGTATCCTTGGTTATCAATAAAATATTTCCTG
Mus musculus Higd2a 3′-UTR
NM_025933
(SEQ ID NO: 189)
GTATAGCCGGGTCTTAAAGCGCCATGGAAACCATTACAAAACCCAGGAACAACAGACATCCCTGTC
AGACTTGCTCCCTCCGTTTCAGACCGGACCTTATTGTCATTTGGGTGAGGAAGTGGCCGATTTTGT
AACTGATTTGCGCTTCCACCGCTGCCCCCTCCCGCTCCCAAAATCCCAGGTTCATTTCAGTTGGGT
TGCATGCTTCTATTTGTGATGCGTCCCCTTAATTACTTAATAAAAGCTTATTACACTTG
Mus musculus Trappc6a 3′-UTR
Trappc6a-001 ENSMUST00000002112
(SEQ ID NO: 190)
GGACCCCAGACCCCAGGCTTGCCCTTCCCTAAGCTTAGCCTCGGAATGTGGCACCTGACCCTGCCT
CACTGCTCACCTTTGCAGGTCGCCTTGAAGCTGGAGCTCACAGGCTCTGGGGAGGTCACATGTGCT
TCAGACAAGGGAATGAAAGGGCCGGGAGGGTCCCGGGAGGTGGGACCATCCCCTGAGTTCCAAGTC
AGCATGGAGGGACATTAGGGCATCACCCAGATGACAGATGTTCAGTAAAGGTTCTTTATGTGCAAA
CAGA
Mus musculus Ldhb 3′-UTR
Ldhb-001 ENSMUST00000032373
(SEQ ID NO: 191)
CTGCCAGTCTCTAGGCTGTAGAACACAAACCTCCAATGTGACCATGAACCTTTAGTCTTCAGCCAT
GTATGTAGGTCACAGTTTGCTTCTTCCCTGACATGTGATATGAGCTCACAGATCAAAGCCCAGGCT
TGTTTGATGTTTGCACTAGGAGCTCCTGATCAAATAAAGTTAGCAATTGCAGCATA
Mus musculus Nme2 3′-UTR
Nme2-001 ENSMUST00000021217
(SEQ ID NO: 192)
ACATGAAGAAACCAGAATCCTTTTCAGCACTACTGATGGGTTTCTGGACAGAGCTCTTCATCCCAC
TGACAGGATGGATCATCTTTTCTAAAACAATAAAGACTTTGGAACT
Mus musculus Snrpg 3′-UTR
NM_026506
(SEQ ID NO: 193)
CCTGTGCTCAGCAAGCAGTGTCCACATCCCTCCCCAAAGGCCTGTTTGATTGTGATGTAGAATTAG
GTCATGTACATTTTCATATGGAACTTTTTACTAAATAAACTTTTGTGATACTC
Mus musculus Ndufa2 3′-UTR
NM_010885
(SEQ ID NO: 194)
AGGTCTCCACTGAGGACTGTGAGCGAGAGCAGCTGAACCTGCTGGACTGAAGACAGTGTGGGGAAA
TGTGTGCTTTGGGTCCTTATAAAGCTTACGCTGTACAGTGTCCCTTCAGAATGTCCTCTTCATTAC
CTTCTCCCTCTTACTGCGCAACACTGAGGCAAAGTAGTTTTATATAAAAATACTCCTTTATTTCTC
CTCAAAAAAAAAAAAAAAAAAAACCCACCAGGTGCCA
Mus musculus Serf1 3′-UTR
Serf1-003 ENSMUST00000142155
(SEQ ID NO: 195)
TGACTGGCTTTTTGGAAAACCTGGGTGCTATTGCCAGTGGGTGCATCATACGCTCTAAGATTAAAA
TTTCACAGTGACTAATCATTATATGTGTTATAACTTGTCCTTATAAAACTATTTTAAACTTTACTC
TTCAGCCTATCTTAATGTGATGTTTTAAGACCATCAAAAAATAAAGTACTGACCTTGCATGTAA
Mus musculus Oaz1 3′-UTR
Oaz1-001 ENSMUST00000180036
(SEQ ID NO: 196)
GTGCCAGCCCTGCCCAGTGTCCCTGTGCCCTCTCCTGGGTTAGTCCACATGTCGTGATTGTGCAGA
ATAAACGCTCACTCCATTAGCGGGGTGCTTCTTCGAGCTGAATGCTGTGTTTGTCACACTCAAGTG
TTGGCTTTAATTCTAAATAAAGGTTTCTATTTTACTTTTTTATTGCTGTTTAAGATGGTCAGGTGA
CCTATGCTATAGCAGTCTCCTTTGAAGTCTGGAAAAATAGTGTCACCTCCCCTGGCTCAAATCCAA
TAAAGTGATCTCGTTCATTGGC
Mus musculus Ybx1 3′-UTR
Ybx1-001 ENSMUST00000079644
(SEQ ID NO: 197)
ATGCCGGCTTACCATCTCTACCATCATCCGGTTTGGTCATCCAACAAGAAGAAATGAATATGAAAT
TCCAGCAATAAGAAATGAACAAAGATTGGAGCTGAAGACCTTAAGTGCTTGCTTTTTGCCCTCTGA
CCAGATAACATTAGAACTATCTGCATTATCTATGCAGCATGGGGTTTTTATTATTTTTACCTAAAG
ATGTCTCTTTTTGGTAATGACAAACGTGTTTTTTAAGAAAAAAAAAAAAAAGGCCTGGTTTTTCTC
AATACACCTTTAACGGTTTTTAAATTGTTTCATATCTGGTCAAGTTGAGATTTTTAAGAACTTCAT
TTTTAATTTGTAATAAAGTTTACAACTTGATTTTTTCAAAAAAGTCAACAAACTGCAAGCACCTGT
TAATAAAGGTCTTAAATAATAA
Mus musculus Ybx1 (v2) 3′-UTR
with mutation T128bpG and deletion de1236-237bp
(SEQ ID NO: 198)
TTTTTATGCCGGCTTACCATCTCTACCATCA
TCCGGTTTGGTCATCCAACAAGAAGAAATGAATATGAAATTCCAGCAATAAGAAATGAAC
AAAGATTGGAGCTGAAGACCTTAAGTGCTTGCTTTTTGCCCGCTGACCAGATAACATTAG
AACTATCTGCATTATCTATGCAGCATGGGGTTTTTATTATTTTTACCTAAAGATGTCTCT
TTTTGGTAATGACAAACGTGTTTTTTAAGAAAAAAAAAAAAGGCCTGGTTTTTCTCAATA
CACCTTTAACGGTTTTTAAATTGTTTCATATCTGGTCAAGTTGAGATTTTTAAGAACTTC
ATTTTTAATTTGTAATAAAGTTTACAACTTGATTTTTTCAAAAAAGTCAACAAACTGCAA
GCACCTGTTAATAAAGGTCTTAAATAATAA
Mus musculus Sepp1 3′-UTR
NM_009155
(SEQ ID NO: 199)
ATTATTTAAAACAAGGCATACCTCTCCCCAACTCAGTCTAAAGACACAATTTCATTTTGAGAATGT
TTACAGCCCATTTAATTAATCAGTGAACTAAAAGTCATAGAAATTGGATTTGTGCAAATGTAGAGA
AATCTACCATATTGGCTTCCAAAATTTAAAAATTTTATGCCACAGAACATTTCATCCAAATCAGAT
TTGTACAATAGGGCACCTGAAAAGTGACTGCAGCCTTTGGTTAATATGTCTTTCTTTTTCCTTTTT
CCAGTGTTCTAGTTACATTAATGAGAACAGAAACATAAACTATGACCTAGGGGTTTCTGTTGGATA
GCTTGTAATTAAGAACGGAGAAAGAACAACAAAGACATATTTTCCAGTTTTTTTTTTCTTTACTTA
AACTCTGAAAACAACAGAAACTTTGTCTTCCTACTCTTACATTCTAAACCGATGAAATCTTTAACA
GATTACACTTTAAATATCTACTCATCATTTTCTCTCTCAGAGTCCTAGCTTGAGTTGCACTGCATG
TATCTGTGCATCTTGTTCTCTTCATTTAATGCTGTACTGTTCTGCTGAGCTCTGAGGGACTATCTT
GAGAGATGTAATGGAAGGAAAGCGTGGTGTTAATCTGCGTACTGCTTAAGACAGTATTTCCATAAT
CAATGATGGTTTCATAGAGAAACTAAGTCCTATGAACCTGACCTCTTTTATGGCTAATACGACTAA
GCAAGAATGGAGTACAGAATTAAGTGGCTACAGTACACACTTATCAAAATAAATGCAATTTTAAAA
CCTTTC
Mus musculus Gaa 3′-UTR
Gaa-001 ENSMUST00000106259
(SEQ ID NO: 200)
GAGAGTCCGTCGTTTACAGAGGCCTCCAGGGAGGCAGAGGGAGCTTGAGCTGGCTCTGGCTGGTGG
CTCCTGTAAGGACCTGCGTCCTGCTCTCCTGACACATCTTTGAGCTTTTCCCACCGTGTTACTGCA
TGCGCCCCTGAAGCTCTGTGTTCTTAGGAGAGTGAGGCTCGCCTCACCTGCCCCACCCCAGCTGTC
TGTCCCTCACCTGGCACTAGAGAATGTGGAGCTCGGCGTGGGGACATCGTGTCTGCACCAACATCA
GGCTGTGCAGCCACTGCAGCCGCAACCCTGCAGAGACAGAGCTGGTGCCTTCACCAGGTTCCCAAG
ACTCGAGAAACTTACTGTGAAGTGTACTTACTTTTAATAAAAAGGATATTGTTTGGAAGC
Homo sapiens ACTR10 3′-UTR
ACTR10-002 ENST00000254286
(SEQ ID NO: 201)
AAGTTTGATTAAAAATCAACCTTGCTTCATATCAAATATTTAACCAATTATAAGCAAATTGTACAA
AGTATGTAGGATGTTTTGTTATAGAGGACTATAGTGGAAGTGAAAGCATTCTGTGTTTACTCTTTG
CATTAATATATAATTCTTTTGACTTTGTTTCTCTTGTGTAGTGGTAAAATGGTAGCTGGTGCTTAT
TGAGATTTGCTGTATTTATATCAATAAAGTATAGTAAAGCAGTTTGATTTTGGAAGTTTGTTATGT
GGCTTTTTTTTTTTTTTTTTTTTTGAGACGGAGTCTCGCTCTGTCACTTAGGCTGGAGTGCAGTGG
CACAATCTCTACTCATTGCAAGCTCCGCCTCCCGGGTTTACGCCATTCTGTCTCAGCCTCCTGAGT
AGCTGGGACTATAGGCATACGCCACCCCGCCCGGCTAATTTTTTGTATATTTAGTAGAGACGGGGT
TTCACCATGTTAGCCAGGA
Homo sapiens PIGF 3′-UTR
NM_173074
(SEQ ID NO: 202)
GTAACTTAATCCTGACAACCGTAGTGCAAGGTATGGCCCATCTCCTGTACGCTTGGAGCGACCTTT
GGCTACGTGGCTGGCCTTGTTATTTCACCACTCTGGATATACTGGAATAGAAAGCAACTTACATAC
AAGAACAATTAACTGGAGCAAAGGGAGATATTTCTTTGTGCAGATTCTGTAAGGGCTGGGCAGAAA
TGTGTATGGTCAAAGCCAAGCAGTTCCATTTACAGCTCTGTTTTTTACGTAGTTACAACATGATGT
GATTGTAGCTTTTTAAACTATGAAACCCCTGAGAGATTGTACCTTCTAGTTGAAATAAAGTATTTA
TAATAGATTGTGGCTTC
Homo sapiens PIGF 3′-UTR
NM_002643.3
(SEQ ID NO: 203)
CTGGAGCAAAGGGAGATATTTCTTTGTGCAGATTCTGTAAGGGCTGGGCAGAAATGTGTATGGTCA
AAGCCAAGCAGTTCCATTTACAGCTCTGTTTTTTACGTAGTTACAACATGATGTGATTGTAGCTTT
TTAAACTATGAAACCCCTGAGAGATTGTACCTTCTAGTTGAAATAAAGTATTTATAATAGATTGTG
TCAAAAAAAAAAAAAAAAAAAAAAAAAAAAAA
Homo sapiens MGST3 3′-UTR
MGST3-001 ENST00000367889
(SEQ ID NO: 204)
AGAATTATAGGGGTTTAAAAACTCTCATTCATTTTAAATGACTTACCTTTATTTCCAGTTACATTT
TTTTTCTAAATATAATAAAAACTTACCTGGCATCAGCCTCATACCTAAAA
Homo sapiens SCP2 3′-UTR
NM_001193599
(SEQ ID NO: 205)
AGAACTCCCTTTGGCTACTTTTGAAAATCAAGATGAGATATATAGATATATATCCATACATTTTAT
TGTCAGAATTTAGACTGAAACTACACATTGGCAAATAGCGTGGGATAGATTTGTTTCTTAATGGGT
GTGACCAATCCTGTTTTTCCTATGCTCTGGGTGAATAGAGCCTGATGGTATACTACTGCTTTGCGG
AATTGCATACAACTGTGCATTACAAAGTTAATATGGTAATTATGGTCTGGGGTAAAATTGAGTTTC
AGAATAAAATTAGGAACAGTAAAATCCAAAGAACTATGTAAACAAAAAAGCTTTTGTTTTGCTTAC
AAAGTATATTTAAGGATTATTCTGCTGAAGATTCAGTTTAAGAGTTTTCCTTGGGAGAACTAAGTA
AGAAACACAATGCCAACAGCTGGCCAGTAATTAGTGTTGTGCACTTCATGTCATTAATCAATTTCT
CAATAGTTCTTAAAATTAGTGAGATTAAAAATCTAAAAATTTTGCATTTCATGCTATCAGAAACAG
TATTTTCTTCCCAAATCAAAATAAAAGAAATATGATCAGAGCTTGAACACAGGCTTATTTTTAAAA
TAAAAATATTTTTAACATGGGTTTCCTTATTGAAAAATCAGTGTATTAGTCATAAAACACCATCAT
TAAGAATAATTGAACAATAAAGTTTGCTTTCAGATGCAGTTTTCAAATTATAATCTCATTTCAATT
TATAACGTTCTCAGTCCTTTGTTATAATTTTCCTTTTTCATGTAAGTTTAATTATCTGCATTTATC
TTTTTTCCTAGTTTTTCTAATACTAATGTTATTTCTTAAAATTCAGTGAGATATAGGATAAAATAA
TGCTTTGAGAAGAATGTTTAATAGAAAATTAAAATAACTTTTTCTGGCCTCTCTT
Homo sapiens SCP2 3′-UTR
SCP2-015 ENST00000435345
(SEQ ID NO: 206)
AGAACTCCCTTTGGCTACTTTTGAAAATCAAGATGAGATATATAGATATATATCCATACATTTTAT
TGTCAGAATTTAGACTGAAACTACACATTGGCAAATAGCGTGGGATAGATTTGTTTCTTAATGGGT
GTGACCAATCCTGTTTTTCCTATGCTCTGGGTGAATAGAGCCTGATGGTATACTACTGCTTTGCGG
AATTGCATACAACTGTGCATTACAAAGTTAATATGGTAATTATGGTCTGGGGTAAAATTGAGTTTC
AGAATAAAATTAGGAACAGTAAAATCCAAAGAACTATGTAAACAAAAAAGCTTTTGTTTTGCTTAC
AAAGTATATTTAAGGATTATTCTGCTGAAGATTCAGTTTAAGAGTTTTCCTTGGGAGAACTAAGTA
AGAAACACAATGC
Homo sapiens HPRT1 3′-UTR
HPRT1-001 ENST00000298556
(SEQ ID NO: 207)
GATGAGAGTTCAAGTTGAGTTTGGAAACATCTGGAGTCCTATTGACATCGCCAGTAAAATTATCAA
TGTTCTAGTTCTGTGGCCATCTGCTTAGTAGAGCTTTTTGCATGTATCTTCTAAGAATTTTATCTG
TTTTGTACTTTAGAAATGTCAGTTGCTGCATTCCTAAACTGTTTATTTGCACTATGAGCCTATAGA
CTATCAGTTCCCTTTGGGCGGATTGTTGTTTAACTTGTAAATGAAAAAATTCTCTTAAACCACAGC
ACTATTGAGTGAAACATTGAACTCATATCTGTAAGAAATAAAGAGAAGATATATTAGTTTTTTAAT
TGGTATTTTAATTTTTATATATGCAGGAAAGAATAGAAGTGATTGAATATTGTTAATTATACCACC
GTGTGTTAGAAAAGTAAGAAGCAGTCAATTTTCACATCAAAGACAGCATCTAAGAAGTTTTGTTCT
GTCCTGGAATTATTTTAGTAGTGTTTCAGTAATGTTGACTGTATTTTCCAACTTGTTCAAATTATT
ACCAGTGAATCTTTGTCAGCAGTTCCCTTTTAAATGCAAATCAATAAATTCCCAAAAATTTAA
ACSF2
Homo sapiens
(SEQ ID NO: 208)
ATAAAGCAGCAGGCCTGTCCTGGCCGGTTGGCTTGACTCTCTCCTGTCAGAATGCAACCTGGCTTT
ATGCACCTAGATGTCCCCAGCACCCAGTTCTGAGCCAGGCACATCAAATGTCAAGGAATTGACTGA
ACGAACTAAGAGCTCCTGGATGGGTCCGGGAACTCGCCTGGGCACAAGGTGCCAAAAGGCAGGCAG
CCTGCCCAGGCCCTCCCTCCTGTCCATCCCCCACATTCCCCTGTCTGTCCTTGTGATTTGGCATAA
AGAGCTTCTGTTTTCTTTG
Homo sapiens VPS13A 3′-UTR
NM_033305
(SEQ ID NO: 209)
AATTCATATGTTCTTTATTTTACTTGGAATGTTTCATTAACATGTTTTGTATGACTTATACCATAA
TGCCCATATGTCCATTTATAGGGAGGTAAAACACATTTTCTTTTAAAATGTTTTCCTACACATTTT
CATAAAGCAAAATAATTGTATTATTTAAGCACAGAAAAAAATGTATCTTACATCCAAAGTAGGGAG
GGCATCCAACATATTATAGATTTGCTTTTATATATTTTATAGCTTTGTATTGCATAGTTTGTCTTT
AAGAGTTCAAGTTAGACTTAAATATAATTTTGATGTTCACTGGTTTTATTTTAAATTGCCTTCTTA
TTTGTTAGCAAAATGCCTTTTTTTAATGGTCTCTGTAAATTTTCTGGGCTTTAATGTAATGCCACT
GTGTAAAAAAAAAGGAAGAAAATAGTAATAGCCATTTAATGTTTTATATTTATCATTTTAAAGATA
TTTTTGTCAAATTTCTTTTAATAATAATAAACATATGTAATCTAAAAAAAAAAAAAAAAAAAAAAA
AAAAAAAAAAAAAAAAAAAAAAAAAAA
Homo sapiens CTH 3′-UTR
NM_001190463.1
(SEQ ID NO: 210)
TATTCCAGAGCTGCTATTAGAAGCTGCTTCCTGTGAAGATCAAATCTTCCTGAGTAATTAAATGGA
CCAACAATGAGCCTTTGCAAAATTTTCAAGCGGAAATTTTAAGGCACCTCATTATCTTTCATAACT
GTAATTTTCTTAGGGATCATCTCTGTTAAAAAGTTTTCTGTATGTCATGTTATAATTACAGGTCAA
TTCTGTTAATATCTTTTTGTTAATTTTGCTCTATGTTTGCCTCTGAAGGAGGTGAGATTTGTGCTA
CTTTGGGAGATTATGTTCTTTTTTCATGTCTAAGATTTATTTTGATCATGTTTATAATATAATGGT
AATTCATTTTTGATGTTTTGTGAAGAATTTAAATTTAAACGAATGTTCTTAAATCAAGTGTGATTT
TTTTGCATATCATTGAAAAGAACATTAAAAGCAATGGTTTACACTTAGTTACCATAAGCCGAAAAT
CAAATACTTGAAAAGTTTACTGTGAAATTCTACTGATTTAAGACTATACTTAATATTTTTAAAAAA
ATAAATCAGCTGGGCGCGGTGGCTCACGCATGTAATGCCAGCACTTTTGGAGGATAAGGCGGGCGG
ATCACGAGGTCAGGAGATTGAGACCATCCTGGCTAGCGCAGTGAAACCCCCATCTCTACTAAAAAT
GCAAAAAAAATTAGACGGACGTGGTGGCGGGTGCCTGTAGTCCCAGCTACTTGGGAGGCTGAGG
Homo sapiens CTH 3′-UTR
CTH-001 ENST00000370938
(SEQ ID NO: 211)
TATTCCAGAGCTGCTATTAGAAGCTGCTTCCTGTGAAGATCAAATCTTCCTGAGTAATTAAATGGA
CCAACAATGAGCCTTTGCAAAATTTTCAAGCGGAAATTTTAAGGCACCTCATTATCTTTCATAACT
GTAATTTTCTTAGGGATCATCTCTGTTAAAAAGTTTTCTGTATGTCATGTTATAATTACAGGTCAA
TTCTGTTAATATCTTTTTGTTAATTTTGCTCTATGTTTGCCTCTGAAGGAGGTGAGATTTGTGCTA
CTTTGGGAGATTATGTTCTTTTTTCATGTCTAAGATTTATTTTGATCATGTTTATAATATAATGGT
AATTCATTTTTGATGTTTTGTGAAGAATTTAAATTTAAACGAATGTTCTTAAATCAAGTGTGATTT
TTTTGCATATCATTGAAAAGAACATTAAAAGCAATGGTTTACACTTA
Homo sapiens CTH 3′-UTR
CTH-002 ENST00000346806
(SEQ ID NO: 212)
TATTCCAGAGCTGCTATTAGAAGCTGCTTCCTGTGAAGATCAAATCTTCCTGAGTAATTAAATGGA
CCAACAATGAG
Homo sapiens NXT2 3′-UTR
NXT2-004 ENST00000372107
(SEQ ID NO: 213)
AGGGGCAAAAGTCCATTCTCATTTGGTCCATTAGTTCCAGCAATTGAAATTTATGTGAATTATTTT
GATTGTAGAAGCACTATAATATGTGCTGAAACTAAATTTCTTTAATATTTTCTATTCCTGTCAGCA
CCTTTTCTAGCAGCTGCCAGTTTGGAGCATTGCCCTCTAAGAGCTTTAAAACTATTTTTTTACATG
CCTTATATACATTCCACTAATGACATTCTTATAATAATATTAAACACATGATCTTGGTACTAACAT
ACTCACTGTGAACCCAGCCTAT
Homo sapiens MGST2 3′-UTR
NM_002413
(SEQ ID NO: 214)
CTTTTTCTCTTCCCTTTAATGCTTGCAGAAGCTGTTCCCACCATGAAGGTAATATGGTATCATTTG
TTAAATAAAAATAAAGTCTTTATTCTGTTTTTCTTGAAAAAAAAAAAAAAAAAAA
Homo sapiens MGST2 3′-UTR
NM_001204366.1
(SEQ ID NO: 215)
CTTTTTCTCTTCCCTTTAATGCTTGCAGAAGCTGTTCCCACCATGAAGGCTTGAAGCCACAGTGCA
TGGCCAGAACCAGCCAGACCTTTGGAGTTCAAGAACTCGAGAGGTGGGTGAAAACTGCCATTGCCT
CCACAGACTGTCTTCTCCGTGGAAAGAAGACCTGAGTCACCAGGGCTGGGAAACCTGCACCACTGA
GACGAGCACAGCCTCTGCCGGCATGCAAGTGGCCGCTGTCAGGACACATGGACTGAAAGTGGTTTG
TCAGCTGCTCCATTAGGTTTTTTTTACCCATATGTTTGCTACCTTTCTTTCCTTGATTTAAAAATA
GGGAGGGGGAGCAGTCTCAGCTGTCTTCAGCTGCTAGGGAGATTTTTTTCCCCCTCCTGAGCTACT
GTTTCCCCCAACCCGAGCCTTTCTCTCTTATTGTACCCACCCTTTCTGATGAAGTCATCAAAGCAA
AGATTGCATAACTGATGCATAGGCCTATCTTGTGTTATACTGGGAGACAGGCCAATGTTTCCATTA
ATAGACAAGAGCACCACCACGCTGCCAAATGGAGCTCTCTGCTGCAACCACTAC
Homo sapiens C11orf67 3′-UTR
AAMDC-005 ENST00000526415
(SEQ ID NO: 216)
TGGAGCCTTAAGAGGAGAATAAATCACTAAGTGCCTA
Homo sapiens PCCA 3′-UTR
NM_000282
(SEQ ID NO: 217)
AGGATTTATAACCTTTCAGTCATCACCCAATTTAATTAGCCATTTGCATGATGCTTTCACACACAA
TTGATTCAAGCATTATACAGGAACACCCCTGTGCAGCTACGTTTACGTCGTCATTTATTCCACAGA
GTCAAGACCAATATTCTGCCAAAAAATCACCAATGGAAATTTTCATTGATATAAATACTTGTACAT
ATGATTTGTACTTCTGCTGTGAGATTCCCTAGTGTCAAAATTAAATCAATAAAACTGAGCATTTGT
CT
Homo sapiens GLMN 3′-UTR
NM_053274
(SEQ ID NO: 218)
AAGTTCCATTTCCTAAATAAAAACTAATAAAATATAGTACTTTCCATTATGATTCATTTAATACCT
TTATAAAAAATTTTTCTGTAAAAATTTACTGCTTGAAAAATAAATGTAGCTTTTCTCATTTATCAA
AAAAAAAAAA
Homo sapiens DHRS1 3′-UTR
NM_001136050
(SEQ ID NO: 219)
CCCTCCTGGTCTGACACTACGTCTCTGCTTGTCTTCTCATTTGGACTTGGTGGTTCGTCCTGTCTC
AGTGAAACAGCAGCCTTTCTTGTTTACCCATACCCTTGATATGAAGAGAAGCCCTCTGCTGTGTGT
CCGTGGTGAGTTCTGGGGTGCGCCTAGGTCCCTTCTTTGTGCCTTGGTTTTCCTTGTCCTTCTTTT
TACTTTTTGCCTTAGTATTGAAAAATGCTCTTGGAGCTAATAAAAGTCTCATTTCTCTTTCAAAAA
AAAAAAAAAAAA
Homo sapiens PON2 3′-UTR
PON2-001 ENST00000433091
(SEQ ID NO: 220)
ATTGTACTTTTGGCATGAAAGTGCGATAACTTAACAATTAATTTTCTATGAATTGCTAATTCTGAG
GGAATTTAACCAGCAACATTGACCCAGAAATGTATGGCATGTGTAGTTAATTTTATTCCAGTAAGG
AACGGCCCTTTTAGTTCTTAGAGCACTTTTAACAAAAAAGGAAAATGAACAGGTTCTTTAAAATGC
CAAGCAAGGGACAGAAAAGAAAGCTGCTTTCGAATAAAGTGAATACATTTTGCACAAAGTAAGCCT
CACCTTTGCCTTCCAACTGCCAGAACATGGATTCCACTGAAATAGAGTGAATTATATTTCCTTAAA
ATGTGAGTGACCTCACTTCTGGCACTGTGACTACTATGGCTGTTTAGAACTACTGATAACGTATTT
TGATGTTTTGTACTTACATCTTTGTTTACCATTAAAAAGTTGGAGTTATATTAA
Homo sapiens NME7 3′-UTR
NM_013330
(SEQ ID NO: 221)
TGGTGTGGAAAGTAAAGAAGTCACAGGTTGGGACATTTAGACAAGAGTGAATCACACACGAGGAAT
GTGTTCATTCTTTTATTGTCCGTTGTTTTAACCTGACTGAATACAAGATCAACAAGAGCACTGTAC
TCCTGGCAATTATTACATATGTTAGAACATGGATTTTGCACTGTAGACAACATTTAACACCAGTCT
ATGGGGTACTGCATTGCTTTTTATAAAGTTCAAAATAAAGATTTATTTTCAAACAAAAAAAAAAAA
AAAAAAAAAAAAA
Homo sapiens ETFDH 3′-UTR
NM_004453
(SEQ ID NO: 222)
ACTGCAGCTAGCCAGTTTCTTTCAAGTATGGCAAGCTAACGTTAAAATGTTTAGAGATTAACAGAT
TTCAGAATGTCTTTCTGCATATTACTGAACAGAATAGTCACAAAATGATTATCAAATAAAAATTTT
ATACTATATGTAAGATTGTCCCATAAAGAAA
Homo sapiens ALG13 3′-UTR
BC117377
(SEQ ID NO: 223)
GATCCAGCAGTATGAAGTATTCTTGCACTGCCATTTTCTTGCTGTTTTTGTTTTTAAAAAGTATTT
TATGTTAGTGGTTAAATGATTTAGGTGATTAGTGTTTACTATTGTATTTGTCTTTAAAATTATTTT
ATCTTTTGATTTAAAATAGTACTTTAAAATTAAGGGGTATTATTTTGGGCTGTGACTAAGGAAATT
GAGATGGATGTACAACTAGCCCCATATTGAGCATACTTCATTGTATTCAGCTGTTTTCCTGTCAGC
CATTTGTCAGC
Homo sapiens ALG13 3′-UTR
NM_001099922.2
(SEQ ID NO: 224)
GATCCAGCAGTATGAAGTATTCTTGCACTGCCATTTTCTTGCTGTTTTTGTTTTTAAAAAGTATTT
TATGTTAGTGGTTAAATGATTTAGGTGATTAGTGTTTACTATTGTATTTGTCTTTAAAATTATTTT
ATCTTTTGATTTAAAATAGTACTTTAAAATTAAGGGGTATTATTTTGGGCTGTGACTAAGGAAATT
GAGATGGATGTACAACTAGCCCCATATTGAGCATACTTCATTGTATTCAGCTGTTTTCCTGTCAGC
CATTTGTCAGCTTTATATTAGCTGATGGTACCAATTGATAAAATGAATATAAAGTATTTCATTGGT
TCAAAAATCACACATCATATTAAACCATGCAGAATTGGAGTAACTTCCACTTTTTTCTAGAAAGTA
AAACCAAGAGCCTTTGCTTCTGGATAACTCACTTAATATTAAATTAAAGAGCTCTTCACGTTTCTT
GAGAATTATCTGAAGCCAGTTGCATTCTGTGATATCAGTTTTGAAGGCACATGGTTCTCTGCTTTA
GATTTATCCCATATGCTATTGTTTAATACTGGATGTATGTAAGTGTTTTACTGCACTGTATTGAAT
TGGTGTCTTTTGCACAGTTAGCAGTAAATAAAAATTAGCATTTAAAATTGCCAAAAAAAAAAAAAA
AAAA
Homo sapiens DDX60 3′-UTR
DDX60-001 ENST00000393743
(SEQ ID NO: 225)
AAACAAAGTCTATGCAAACCACTTAAAAATAATTCCATAGTAGTTTTTCAGGTCACGTTTTTGATT
CTTATGCTTCTTGCCAGAAATACATTATGATAAAGTGGAAATACATTACGATGAAGTGGAAAGAGC
AAACACTTTGGAATCAAACAGAGTTGCAATCAAACCTGCCATGTTCTGTCATGAATACTCACAAAT
TATTTAGTATACCTGAATCTTGGTTTCTTTTTATAACTGAGTAATAATGGTTACATCTCAGGTAGT
TTGAGGATTGACTAAAAAAATGCGAGAATGTTGTATGTGACTGAATAACAATTTTTACTCTGCGAA
GCCAAAGTAAATATAATATTATCAGTAACTTTATCCCCAGTGTCAGTATTTATAAAATGTTTATTA
AGGCTAGAAAAAATGAATACAATATCCTGAAGGTGAAATATATTCTCTTCAATTAGCATAAATATG
ATTTACATAAGTTAGCTATACAGCTATTGAGATAGTACTTTCTAGTAAACTTAAACTACTTTTTAA
ACATACATTTTGTGATGATTTAACAAAAATATAGAGAATGATTTGCTTTATTGTAATTGTATATAA
GTGACTGGAAAAGCACAAAGAAATAAAGTGGGTTCGATCTGTTTAC
Homo sapiens DYNC2LI1 3′-UTR
NM_015522.3
(SEQ ID NO: 226)
AATTCATTTGATGTAGATGAACCTGTTCACTGGAAAATTACAGCAATTTATTAAAACCTCAGTAAG
AGCAAAACAAGGAAGAAGATTCCTTATATCTTCTTGTTAGACATCTTCTGTGATTGTTATGGCATA
TTACACCAATCAGAGAAATAGAGTTTTAAAGTAGTGGTTTGATATTGATTTTATAATCTCTGTAAA
AATGAAGATAAAAAGCCAGATTGTACAAAAGTCACCTGACAAAGACTAGATGAAGCTACAACTTTA
AGCAAGGGGTAGAGTTGTAATAGCCTTCACCATCACTCTGTATTTTACATTCATTTCGTTTCTGTC
ACTTATTCAGTATCTTTTTATCATCTGACAGCTAATTAAATTATAAAGTTGCTATGATGGTAACAC
AAGTTCTTCAAATACAATAATAAATATCATCATCTGGAAAAAAAAAAAAAAAAAA
Homo sapiens VPS8 3′-UTR
NM_001009921, NM_015303
(SEQ ID NO: 227)
TGACTCCATGGAGCCTGGCCCAGGAGAACCAGAGATGATCCCGAGGCAGCTGGGGAGAGGCCCCGC
CTCTGGTGGGCTTGGCCTCCACCACCTCCCACGCTTCTGAGAAGAGGTTCCAAATTGGGCTTCTGT
GCCCAGAGCGTCCACAGCACCATTCCCAGTGTAGACTCCCAGTCTTCTCCACATTGCTGTCATGGC
GTCAGTTCACCAGACTCATTGATTTTGTTTTGCTTGTTAAGCAAAGGAATGTCACATACCTCTGTC
CAGCTTTTTAGGAAATACATTTCGCCTATTGCGACTTTTTCCATTTACCCTGAAGCCTAGAAAGTA
GGTGGAACTCACACAAATGGCATTCCAGAGTCTGCCATACTCCGTCTCCTCCAGCTGCTGGATAAT
ACAGAGGAACTTCAACTTCTACAGGGAACAGTGGTTGGCCAGGCTGCAGTATAACTGAAGCATGCC
TTGGAGAGAGCAGACACTGTGGGGGCCAGGGCCATCTCCCTTTAATGTGTTCATGTTAAAACCTAT
TTGAGTGTAAGACTTGCCCTTTCTAACAATAAATGCTCCGTGTTTAAGTTCTGCAGGTCTCAAAAA
Homo sapiens ITFG1 3′-UTR
NM_030790
(SEQ ID NO: 228)
CTTGCCTTTAATATTACATAATGGAATGGCTGTTCACTTGATTAGTTGAAACACAAATTCTGGCTT
GAAAAAATAGGGGAGATTAAATATTATTTATAAATGATGTATCCCATGGTAATTATTGGAAAGTAT
TCAAATAAATATGGTTTGAATATGTCACAAGGTCTTTTTTTTTAAAGCACTTTGTATATAAAAATT
TGGGTTCTCTATTCTGTAGTGCTGTACATTTTTGTTCCTTTGTGGAATGTGTTGCATGTACTCCAG
TGTTTGTGTATTTATAATCTTATTTGCATCATGATGATGGAAAAAGTTGTGTAAATAAAAATAATT
AAAAAAAAAAAAAAAAAAAAAAAAAAAAAA
Homo sapiens CDK5 3′-UTR
NM_004935
(SEQ ID NO: 229)
GCCCCGGGACCCCCGGCCTCCAGGCTGGGGCCTGGCCTATTTAAGCCCCCTCTTGAGAGGGGTGAG
ACAGTGGGGGTGCCTGGTGCGCTGTGCTCCAGCAGTGCTGGGCCCAGCCGGGGTGGGGTGCCTGAG
CCCGAATTTCTCACTCCCTTTGTGGACTTTATTTAATTTCATAAATTGGCTCCTTTCCCACAGTCA
AAAAAAAAAAAAAAAAA
Homo sapiens C1orf112 3′-UTR
BC091516
(SEQ ID NO: 230)
AACTTATCACTAGGCAGAACTGGGTTTGATGCTTTGTCAACTGAAAATACTTATGTCTGTACATTT
TCTAACAGATATAAAACAAATTTTGTAAAGTTGAAAAAAAAAAAAAAAAAAAAAAAAAAAAAAAAA
AAAAAAAAAAAAAA
Homo sapiens IFT52 3′-UTR
NM_016004
(SEQ ID NO: 231)
AGACCATGCCTCTTGAAGCTTTTTCTGCCTCCTGATTCTCTCTTTGTAAACTATTTTCAAATTGTT
TTTCAACTCCTTATCAAAATTGTTTATACACTCTTTCCTCCATGAGCTCTGGAAGGTATATGCATC
TTCTGTAATACTCAGATAGGTATAAGATTTTTCACAAAATCCTTATGTAAGATACATTCCATTTTT
AAAAATTAAATGTATGGTTGCATCTGTCTTTTTATACCCTA
Homo sapiens CLYBL 3′-UTR
CLYBL-003 ENST00000339105
(SEQ ID NO: 232)
TCTGTTAAATGAAGCTGTCATCAGGCTAAAGGGTATTGAAGCTGCAGAGGGATCAACTTGTGCTTG
CCAGAGGACGCCAATGAAGTTTGAAACACCAACAATCAGAGATTTTGTTTCTGTTCCTCATTAAAT
CATGAGCTTTTGTG
Homo sapiens FAM114A2 3′-UTR
FAM114A2-006 ENST00000520667
(SEQ ID NO: 233)
AGAATGGAGACGTTTTGACCTGGGACTTGTGACGGCCAAGGAATGCCACCTTATTCTGGCTACTCC
TGCAGAAATGAAGGAGTGGGGTTATTTTAGTATATAAAAATTCAGGCAGGAGAGATGGTTTAAAGA
GGAAGATTGTTGCCTTCAGTGTTTGATTGAAGTATTCAGGTTCTCACAGTATTCTTTCCAGTTGTT
GTAATTCATAAATTATTTGAAAAGAAACTTTTGTAGAAAGTCCAAGAATAATAACTCTAGATAAAG
ATTAGTGGGACACTCAGGCAAAAATGTTGGTCTTTCTTTGACATGTTGCAAAATGTTATCAATTTT
GTCATGGATATAATTTGCAGCCCATGGATATAACTGGTTGATAAGCCAGAGAAAAATAATTTAGTG
TTCTAAAATTCATGGCATGTGTGGTTTATTAATGCCATGTACTTTCTCCTTTCTGGAATAAAATCT
ATGGCTTTAAGAAAA
Homo sapiens NUDT7 3′-UTR
NM_001243661
(SEQ ID NO: 234)
TTTACTAGAGCAAGAGACAAAGAACTATTCACGAGGATTCTGTGTGTGCTTATTCGTAGAACAACA
ACAATGCCAGCTGTTGGAATTTGACAGGTGTGAATATTTTTTCTGCAGTATGTAGTTAGAATCCTT
GCCTCTTTTCCAGTTGCCTTCTATTGTCTGAAAAAGTAAAAGCCATTCAAAAATGAAAACTATGTT
CATAGTGTTGCATATTTTCACCCACAATATGTTAATAATATTTTTCTTACACATATAATAAAGAAT
ATCTGGCACATACTAGGCCCTTAATAAAGATTTTTTGAATATATAA
Homo sapiens AKD1 3′-UTR
NM_001145128
(SEQ ID NO: 235)
TTTACTTAGGTGATAGCAGCCTGAATCTCAAGAGTTATCTGAAAGTGATAGAGGGAAACTGAGAGA
AGTAGATTGAAAATCTGGGCCTCTTGGAAGTACTTTTGCCTCCTGAGCAAGGTACCATGGCTGCCA
GACTTCAGGTGAACTCAAAGGTCTGCCAGCCAGGAAGGAGCACTCTTATGGAAACAAGTTTTAATA
CAATTTTAAAATGTATTGCTCTTTGCCTGAACTTTGATGCTTTAACAAAATAAACATTCTATTTAT
AATTCCATATAGAAAAGTTAAGTGACTTATTTAATAAATGTATTATTTTCCTTTTTAACATTTTCA
GTAGAAAAGTCAGTCTCTGTTAAAATTACTCATTAAATGTTAGAAAGCTTTAAGACATTTAACATT
GTTATAAATGAAACCAAAATATGGGTTATACATTTTACATACAAAACTGTTTGTGAACTTTGTGAA
CATAAGATACTATCATTTTCCCAATAAAATAAATGGATTTTGCAACAACTT
Homo sapiens MAGED2 3′-UTR
NM_014599
(SEQ ID NO: 236)
GATTTTAGATATTGTTAATCCTGCCAGTCTTTCTCTTCAAGCCAGGGTGCATCCTCAGAAACCTAC
TCAACACAGCACTCTAGGCAGCCACTATCAATCAATTGAAGTTGACACTCTGCATTAAATCTATTT
GCCATTTCAAAAAAAAAAAAAAAAAAAA
Homo sapiens HRSP12 3′-UTR
HRSP12-001 ENST00000254878
(SEQ ID NO: 237)
GTGGGCCCAGTGCTGTGTAGTCTGGAATTGTTAACATTTTAATTTTTACAATTGATGTAACATCTT
AATTAACCTTTTAATTTTCACAATTGATGACAGTGTGAGTTTGATGAAAATATCTGAAGCTATTAT
GGAAATACCATGTAATAGGGAGAGTTGAACATGAATATTAGAGAAGGAATCCAGTTACTTTTTTAA
ATTACACCTGTGTGCACCTGTATTACTGAATATAGGAAAGAGATACCCATTACATAGTTACTCAGT
AAACAAAAGAGAAATACCAGGTAGGAAAGAAGAGTTACTATTCCTGAGAAATAATCAAGAACATAT
TTAATTTAAACTAATGATGTGAACTATTTAGTTTTGATGTCCGTTATGTGATTCTGCTTTTACTTG
AGTAAAATTAAAGTGTTTAAATTTGAGATCAAGGAGAAGATAGTGGAACAAAATGTTATATAGATA
ATATTTTTCTAATGGAAATAAAATAGGCAGATTTCC
Homo sapiens STX8 NM_004853 3′-UTR
(SEQ ID NO: 238)
TGGCAGTAAAGAGACCACCAGCAGTGACACCTGCCAATGACAGATGCAAGCCCAACACCCTTTTGG
TACGCAAAACCTGCTCTCAATAAATTCCCCCAAAGCTCTGAAAAAAAAAAAAAAAAAAAAA
Homo sapiens ACAT1 3′-UTR
ACAT1-001 ENST00000265838
(SEQ ID NO: 239)
ACAACCTCTGCTATTTAAGGAGACAACCCTATGTGACCAGAAGGCCTGCTGTAATCAGTGTGACTA
CTGTGGGTCAGCTTATATTCAGATAAGCTGTTTCATTTTTTATTATTTTCTATGTTAACTTTTAAA
AATCAAAATGATGAAATCCCAAAACATTTTGAAATTAAAAATAAATTTCTTCTTCTGCTTTTTTCT
TGGTAACCTTGAAAAGTTTGATACATTTTTGCATTCTGAGTCTATACTTATCGAAATATGGTAGAA
ATACCAATGTGTAATATTAGTGACTTACATAAGTAGCTAGAAGTTTCCATTTGTGAGAACACATTT
ATATTTTTGAGGATTGTTAAAGGTCAAGTGAATGCTCTTTATAGGTAATTTACATT
Homo sapiens IFT74 3′-UTR
IFT74-201 ENST00000433700
(SEQ ID NO: 240)
GTTTAAGTCCACTGAAAGTCTCTAAGGAAGTATCCTCTTGCTGCTAAACTTGGTACAAGTTGACTA
CCAAAAAAAAAAAAAGCTTACTTTTGGAGTTTACCTAAAATTTCTGAATGTTATAATTTTTGTGGC
CTCTTTTAAGAATGATATTTTAAAATAGTAAATAGTTCAATAAATGGTTTGCATATT
Homo sapiens KIFAP3 3′-UTR
NM_014970
(SEQ ID NO: 241)
TAAAGTATCTGTTTCCATGTGTAATCTCAGCTTAGAAGAAATCTGTGTGGGTTGGGTTAATTTTGG
ATCTTTGCCTAATAATGCATGTTGATGTTATTGTGGGTCTGTGTTTGTTTTTATTTTTATATGTTG
TTAGCTGCAGATTAACCCCAGCCCCTCTGTCTTCTGTTAAGTACAGTTGATACTGACATTGTTCAC
TCATCAAACCACATCTTGATGCTAAGTAACATTTCCCATGAGCCACAAAACTGAATGCTGAAAAGC
TACTAGACTGGAAAACAAACACTGCATTATGTATGTTAAGTGACTAATTTAATTTCAATTAAAAAG
CGTAAAGTGAAAATGAAAAAAAAAAAAAAAA
Homo sapiens CAPN1 3′-UTR
NM_005186
(SEQ ID NO: 242)
GGCAGGGACTCGGTCCCCCTTGCCGTGCTCCCCTCCCTCCTCGTCTGCCAAGCCTCGCCTCCTACC
ACACCACACCAGGCCACCCCAGCTGCAAGTGCCTTCCTTGGAGCAGAGAGGCAGCCTCGTCCTCCT
GTCCCCTCTCCTCCCAGCCACCATCGTTCATCTGCTCCGGGCAGAACTGTGTGGCCCCTGCCTGTG
CCAGCCATGGGCTCGGGATGGACTCCCTGGGCCCCACCCATTGCCAAGCCAGGAAGGCAGCTTTCG
CTTGTTCCTGCCTCGGGACAGCCCCGGGTTTCCCCAGCATCCTGATGTGTCCCCTCTCCCCACTTC
AGAGGCCACCCACTCAGCACCACCGGCCTGGCCTTGCCTGCAGACTATAAACTATAACCACTAGCT
CGACACAGTCTGCAGTCCAGGCGTGTGGAGCCGCCTCCCGGCTCGGGGAGGCCCCGGGGCTGGGAA
CGCCTGTGCCTTCCTGCGCCGAAGCCAACGCCCCCTCTGTCCTTCCCTGGCCCTGCTGCCGACCAG
GAGCTGCCCAGCCTGTGGGCGGTCGGCCTTCCCTCCTTCGCTCCTTTTTTATATTAGTGATTTTAA
AGGGGACTCTTCAGGGACTTGTGTACTGGTTATGGGGGTGCCAGAGGCACTAGGCTTGGGGTGGGG
AGGTCCCGTGTTCCATATAGAGGAACCCCAAATAATAAAAGGCCCCACATCTGTCTGTGAAAAAAA
AAAAAAAAAAAAAAAAAAAAAAAAAAAAAAAAAAAAAAAAAAAAAAAAAAAAAAAAA
Homo sapiens COX11 3′UTR
NM_001162861
(SEQ ID NO: 243)
AGAGTTGGCACCTTTGATGTGGTAGTGAGCTGATCATCCACTTTCTTCTAAAATAAAGAGAAGAAA
ATGGCCAGTAAAAAAAAAAAAA
Homo sapiens GLT8D4 3′-UTR
BC127733
(SEQ ID NO: 244)
ATATTTTGTCTTGTTGCAAGTCAATTAGGTGTCTTGTGAACAAGGAAATACTAATCTCTAAGCTGC
CTGGGTCTTTT
Homo sapiens GLT8D4 3′-UTR
NM_001080393
(SEQ ID NO: 245)
ATATTTTGTCTTGTTGCAAGTCAATTAGGTGTCTTGTGACCAAGGAAATACTAATCTCTAAGCTGC
CTGGGTCTTTTTGTGTGAATATTTAATGGTGCTCCATGACTGTTGAGTTTTAAAAACCTCGTTAAA
TTTTGCCAAATCAGTTGCCCCCAAAAGGGAATATGCTTTTCCTTATTTTTTTTTCTAAAATGCTAT
TTATCTCTAAGGAAAAA
Homo sapiens HACL1 3′-UTR
NM_012260
(SEQ ID NO: 246)
ATAAAGACGCCAGTTGGTGGTCTTGAGTTTTCTCTTTCTTGCAAGATGAAATTTTATTTTCCACAG
CAAAATTACTCTACTGTTAAAATTGTGCAAAATAAAATAAACATTTAAAATGACATTTTACAGTAA
AAAAAAAA
Homo sapiens IFT88 3′-UTR
NM_175605
(SEQ ID NO: 247)
TATTCACTTTAATATTTATTAAAGGAAAGAAATTGCCTTATGAGATCATCCTCATGTTAAACCTTG
GATTAAATATCTAACCTGTAATTATTTTTTTTCACTGTCAAAACTTAAGTAAGTGTATTCTATTCT
GTATGTATGCATTTAAGTTGTTTTTTTCTTTTAAGGAATAAAAACAGGTAAAACTAATACTTTAGG
CCAGTGACTTCCTTAGCTTTTTGAAAACATTGACACACAGGAAGAAATAAATTTCATAACACAAAA
AAAAAAAAA
Homo sapiens IFT88 3′-UTR
IFT88-001 ENST00000351808
(SEQ ID NO: 248)
TATTCACTTTAATATTTATTAAAGGAAAGAAATTGCCTTATGAGATCATCCTCATGTTAAACCTTG
GATTAAATATCTAACCTGTAATTATTTTTTTTCACTGTCAAAACTTAAGTAAGTGTATTCTATTCT
GTATGTATGCATTTAAGTTGTTTTTTTCTTTTAAGGAATAAAAACAGGTAAAACT
Homo sapiens NDUFB3 3′-UTR
NM_002491
(SEQ ID NO: 249)
AGATAATACCTGGAAGCATCATAGTGGTTTCTTAACTCTCCAAAATAAGATTTCTTCTCTGTAGCC
TACTTGTCTGGTTTATCCCTTACAGAATATTAGTAAGATTTAATCAATTAAAATATATATATATGC
CAAAAAAAAAAAAAAAAA
Homo sapiens ANO10 3′-UTR
NM_018075
(SEQ ID NO: 250)
GTGCCCAGCGTGCCCAGCTGCCCTGTTGGCAGAGGCCTGTGTCTGTGCCACACCTGCCACGGTGGC
AGGGGGGGTACCCGGGGCAGCATCGTGGCTCCTGAACCCAGACCCAATGCTTAGCCAAACGAAGTG
GCTCCCATGTGGCAAGCACCCTTCTCAGTTTCGCAGTGGCTTGGCTCGGGATCCTTGGCAGTTCCC
CCAGCCCCACCCTGTCTGCTCCTTCCCAGTTCCTTCCCGGGCCCCACACGCTGCTCCAGCTGCCAA
CTTTGCTGCAGAGCCACTGCCGCCCTTGAGCCTCTCACCATGAGTGAGCCACCAGCTCTCCACGTT
CCCCTCATAGCAGTGTCACTCCCAACCCCACCATGGCCCAGGGACCCGTGGACAGGTTGGGGATGG
GGTGTGTGCCCACTGTGCTCATCACAGGAGCCTCAGTTGAGAGTGAGCGGGGTACAGTAAGGCAGT
GCTTCCCACACTGGACCTCTTTCCTGGTTCTCTTTTGCAATACATTAACAGACCCTTTATCAACAT
AAACAATAGTAACTGAGCTATTAAAGGCAACCTCTCTGACTCCTTCTGCCTAAAAAAAAAA
Homo sapiens ANO10 3′-UTR
ANO10-005 ENST00000451430
(SEQ ID NO: 251)
GTGCCCAGCGTGCCCAGCTGCCCTGTTGGCAGAGGCCTGTGTCTGTGCCACACCTGCCACGGTGGC
AGGGGGGGTACCCGGGGCAGCATCGTGGCTCCTGAACCCAGACCCAATGCTTAGCCAAACGAAGTG
GCTCCCATGTGGCAAGCACCCTTCTCAGTTTCGCAGTGGCTTGGCTCGGGATCCTTGGCAGTTCCC
CCAGCCCCACCCTGTCTGCTCCTTCCCAGTTCCTTCCCGGGCCCCACACGCTGCTCCAGCTGCCA
Homo sapiens ARL6 3′-UTR
NM_032146
(SEQ ID NO: 252)
AAAGATAATAGTTGGAAACCTCAGCAATTTTCAATTCAAGGAATCTATCTAAGACAAATAGAATAC
ATTTTGTAAAAGATGTTTATGCATCAAAAAATATAATTTTCTGCTTGCATTTATGGACTCTGACCT
TTTTAAGAACATAGGACTTCAGGTATGCTAATTTGGCCATTAATTATTTAAAAACTAAATATTCCC
TCAAAAGGGCTCCCTAGAATTATCAAGTTCTTAGTGAAGGTCTACATTTGATTGTACGTAGAATGT
TTAAAAGTCAGTTATAAGCCATCTCATCCCATCATAATTTATGATATGTTTAATATATTTTATTTT
TTAATTGTCTTTTTAAAAAATTTAGTTTATGACTTTGCAGTATGAATTGTGCTTGTGAAAAAGAAC
TTTAAATATTTATAAGGGACCATGGGTAATTAATATATATTCAATTTTTACTATGTGTCACTGTCA
ATAAAATGTAAAATATAATGTGCC
Homo sapiens LPCAT3 3′-UTR
NM_005768
(SEQ ID NO: 253)
TCCATTTCCCTGGTGGCCTGTGCGGGACTGGTGCAGAAACTACTCGTCTCCCTTTTCACAGCACTC
CTTTGCCCCAGAGCAGAGAATGGAAAAGCCAGGGAGGTGGAAGATCGATGCTTCCAGCTGTGCCTC
TGCTGCCAGCCAAGTCTTCATTTGGGGCCAAAGGGGAAACTTTTTTTTGGAGAAGGCGTCTTGCTT
TGTCACCCACGCTGGAATGCAGTGGCGGGATCTCAGCTCACCGCAACCTCCACCTCCTGGGTTCAA
GTGATTTTCCTGCCTCAGCCTCCCAAGTAGCTGGGAATACAGGCACGCCACCATGCCCAGCTAATT
TTTGTATTTTCAGTAGAAACGGGATTTCACCACGTTGGCCAGGCTGGTCTCGAACTCCTGACCGCA
AGTGATCCACCCGCCTCCGCCTCCCAAAGTGCTGGGATTACAGGCGTGAGCCACCGTGCCCGGCCC
AAAGGGGAAACTCTTGTGGGAGGAGCAGAGGGGCTCACATCTCCCCTCTGATTCCCCCATGCACAT
TGCCTTATCTCTCCCCATCTAGCCAGGAATCTATTGTGTTTTTCTTCTGCCAATTTACTATGATTG
TGTATGTGCCGCTACCACCACCCCCCCCATGGGGGGGTGGAGAGGGGTGCAAGGCCCTGCCTGCTC
CACTTTTTCTACCTTGGAACTGTATTAGATAAAATCACTTCTGTTTGTTCAGTTTTTCA
Homo sapiens ABCD3 3′-UTR
NM_001122674
(SEQ ID NO: 254)
AAACCAGACAAATGTATTGGCCAGGCGTGGTGGCTCATGCCTGTAATCCCAGCACTTTGGGAGGCT
GAGATGGGAGGATCGCTTGAATCCAGGAGTTCGAGACAAGCCTGGACAAAAAGCGAGACCCGCTTC
TTTAAAAAATAATAATAAAACA
Homo sapiens COPG2 3′-UTR
NM_012133
(SEQ ID NO: 255)
ATGCTTACTGGACAAGAGGAAACTGATGCACACTACATGGTCAGTGGGCTTTTAGGCTAGTGGCAT
CAGTTTCCCAGAATCAGACTTTTGAAGATGAATGACTTTGGAGAAGCAAATTAAACATTTGGCCCT
GAGCCAGCAGATCAAGCAAATGTCTATCTTTGCGCATGGGTTGTTTTTTTTTTTTTTCTTTTTATT
CTACTTGGTCAGCTTTGGGACGATAGTGCAGCTTTGGGTGATCTTGAAAATCAAATACTATCCTAT
ACTCCAGCTGCTTAACTTCATTTTATTCTTTAATGTGTACCTGAAAGCTCCTGGCAATGCTGGAAA
ATTTTTATCCCAGAGGGGTGGGGGGGAGGGGGGAGGGGAAGCCAGAGTCCACTTTTGTCACAATTC
ATTTTTATTAATAGAAAATAAACACTTATTCCAGTTTCAAAAAAAAAAAAAA
Homo sapiens MIPEP 3′-UTR
NM_005932
(SEQ ID NO: 256)
AAGAAACACTCTACACCTCTTAAATCAAGGTCATGTAGATAATGACTTTGTTATAAATGCTACAGC
TGTGAGAGCTTGTTTCTGATTTCATTGTTCGCTTCTGTAATTCTGAAAAACTTTAAACTGGTAGAA
CTTGGAATAAATAATTTGTTTTAATTAAAAAAAAAAAAAAAAAA
Homo sapiens LEPR 3′-UTR
NM_002303
(SEQ ID NO: 257)
TTTCACTGAAGAAACCTTCAGATTTGTGTTATAATGGGTAATATAAAGTGTAATAGATTATAGTTG
TGGGTGGGAGAGAGAAAAGAAACCAGAGTCAAATTTGAAAATAATTGTTCCAAATGAATGTTGTCT
GTTTGTTCTCTCTTAGTAACATAGACAAAAAATTTGAGAAAGCCTTCATAAGCCTACCAATGTAGA
CACGCTCTTCTATTTTATTCCCAAGCTCTAGTGGGAAGGTCCCTTGTTTCCAGCTAGAAATAAGCC
CAACAGACACCATCTTTTGTGAGATGTAATTGTTTTTTCAGAGGGCGTGTTGTTTTACCTCAAGTT
TTTGTTTTGTACCAACACACACACACACACACATTCTTAACACATGTCCTTGTGTGTTTTGAGAGT
ATATTATGTATTTATATTTTGTGCTATCAGACTGTAGGATTTGAAGTAGGACTTTCCTAAATGTTT
AAGATAAACAGAATTC
Homo sapiens LEPR 3′-UTR
NM_001198688
(SEQ ID NO: 258)
GAAATGCTTGTAGACTACGTCCTACCTCGCTGCCGCACCTGCTCTCCCTGAGGTGTGCACAATG
Homo sapiens C2orf76 3′-UTR
NM_001017927
(SEQ ID NO: 259)
AAACATCTCGAGGGCTTCCTTTTTGCAT
Homo sapiens C2orf76 3′-UTR
C2orf76-001 ENST00000409466
(SEQ ID NO: 260)
AAACATCTCGAGGGCTTCCTTTTTGCATACCTGTATTAAGCTCTTTATTCCACTGCTGAATTTTTG
AAATTGACAAACAAATCTTAAAAAATTAATCCCAGGCTATACTCTTTGAGCTAAAATCTGGTTATT
TCTTTCTCTTCAGGTCTTTCCTTCTCTCTTTCTTTTTCTTTGTTGTTGTAAAATAATATATTATGA
GAAAAACATTTGATCTTTTTAAAGGGAAATAAATTGTTATTAAAAA
Homo sapiens ABCA6 3′-UTR
NM_080284.2
(SEQ ID NO: 261)
AACCTCAAACCTAGTAATTTTTTGTTGATCTCCTATAAACTCATGTTTTATGTAATAATTAATAGT
ATGTTTAATTTTAAAGATCATTTAAAATTAACATCAGGTATATTTTGTAAATTTAGTTAACAAATA
CATAAATTTTAAAATTATTCTTCCTCTCAAACATAGGGGTGATAGCAAACCTGTGATAAAGGCAAT
ACAAAATATTAGTAAAGTCACCCAAAGAGTCAGGCACTGGGTATTGTGGAAATAAAACTATATAAA
CTT
Homo sapiens LY96 3′-UTR
NM_015364.4
(SEQ ID NO: 262)
AATAAATTGAGTATTTAAAAAAAAAAAAAAAAAAAAAAAAAAAAA
Homo sapiens CROT 3′-UTR
NM_001243745.1
(SEQ ID NO: 263)
TGATGATGTTTAAAGAATGATAAATAAAAAGTGCATAGTTTTTATTTTTAAATTATTGCTGTAAAA
ATTTTTACAGTTATTATTGTTATTTTCATAATCCAAAAGAAGGAATGAATCACTTAACTTTGGGAG
TTTTCAGTGGGTGGATTCGGGAACTTGTTAAAATGCAGATTTGCTGGGATAAGTGATTCTGATTCA
CATGGCTGGAATGAGGCCCAGAGATTCTTATTTTAACAATCACTTCATGTGGTTTGGCTGCAGGTA
ATCTGTAGACCATGCTGAAGGAAAACATTTTGTCCAGGTGACTAGCTTGAAAAATCAGAAACACTA
AAATAGACATGTCACATAGGTGGCATAGAAATATTTTCGTAGTACAATGGAGAAAGGGAATCATTA
AAAATCAGAGTGGAGAATGGTTATGTATATTGTATATTTCAGTTAGATAAATTGAGGAAGCTAGTA
TAATAATTATTGAAGGTCTCAATAATTTTCCACAAAATTCTTTAACTTCTTCAGCTCAACCATTTC
TGTACTTCTCTACTATGAATCAGAGGATGAGGTTGTATAATTCAAAAGCATTGCCTTAGTCTAGAA
ATAATTATTGTACCTATCATTTAGTTTTAGAAATAAAAAGCAAGCTGATTTTTTTTGATGAACCAT
TTATATCTGTGATGGAATAATAAAATTTCACACTTCCGGATTCCTTTGTTCTCAATTTTGAGCCTT
GAGTTGTTTTAATTAAAGAGGGGTAAAGG
Homo sapiens ENPP5 3′-UTR
ENPP5-002 ENST00000230565
(SEQ ID NO: 264)
TGTTACTTTGAAGTGGATTTGCATATTGAAGTGGAGATTCCATAATTATGTCAGTGTTTAAAGGTT
TCAAATTCTGGGAAACCAGTTCCAAACATTTGCAGAAACCATTAAGCAGTTACATATTTAGGTATA
CACACACACACACACACACATACACACACACGGACCAAAATACTTACACCTGCAAAGGAATAAAGA
TGTGAGAGTATGTCTCCATTGTTCACTGTAGCATAGGGATAGATAAGATCCTGCTTTATTTGGACT
TGGCGCAGATAATGTATATATTTAGCAACTTTGCACTATGTAAAGTACCTTATGTATTGCACTTTA
AATTTCTCTCCTGATGGGTACTTTAATTTGAAATGCACTTTATGCACAGTTATGTCTTATAACTTG
ATTGAAAATGACAACTTTTTGCACCCATGTCACAGAATACTTGTTACGCATTGTTCAAACTGAAGG
AAATTTCTAATAATCCCGAATAATGAACGTAGAAATCTATCTCCATAAATTGAGAGAAGAAGAAGG
TGATAAGTGTTGAAAATTAAATGTGATAACCTTTGAACCTTGAATTTTGGAGATGTATTCCCAACA
GCAGAATGCAACTGTGGGCATTTCTTGTCTTATTTCTTTCCAGAGAACGTGGTTTTCATTTATTTT
TCCCTCAAAAGAGAGTCAAATACTGACAGATTCGTTCTAAATATATTGTTTCTGTCATAAAATTAT
TGTGATTTCCTGATGAGTCATATTACTGTGATTTTCATAATAATGAAGACACCATGAATATACTTT
TTTTCTATATAGTTCAGCAATGGCCTGAATAGAAGCAACCAGGCACCATCTCAGCAATGTTTTCTC
TTGTTTGTAATTATTTGCTCCTTTGAAAATTAAATCACTATTAATTACATTAA
Homo sapiens SERPINB7 3′-UTR
SERPINB7-203 ENST00000546027
(SEQ ID NO: 265)
AAATCCAATTGGTTTCTGTTATAGCAGTCCCCACAACATCAAAGAACCACCACAAGTCAATAGATT
TGAGTTTAATTGGAAAAATGTGGTGTTTCCTTTGAGTTTATTTCTTCCTAACATTGGTCAGCAGAT
GACACTGGTGACTTGACCCTTCCTAGACACCTGGTTGATTGTCCTGATCCCTGCTCTTAGCATTCT
ACCACCATGTGTCTCACCCATTTCTAATTTCATTGTCTTTCTTCCCACGCTCATTTCTATCATTCT
CCCCCATGACCCGTCTGGAAATTATGGAGAGTGCTC
Homo sapiens TCP11L2 3′-UTR
NM_152772
(SEQ ID NO: 266)
AGAAGAACTGACATTGGACGAGAGATTGGAAATCCAGTACTTTGGTATCCAGTCCACTTCCATTGA
TGGCATTAGAGATCCAGCACATTCTCAGTACTGTGGTGCAGTATTAGCCCAAATCTGTGTAATGGG
TAATATTAGCATTACAGAAGACACACACATCACATAGACCCTCAGAAGACGTAAACATCACATAGA
CCCTATTTGTGCATCATTTTCAAGTTTAAAACAGATATTTGTAATGAACAGAAAACAATTTGTAAT
TAATTATATTACCTATATAATACTTGTAAATGTTTTCTTAACCATTTATATTTGGCTTATGACATT
TAACCCCTAAGGAGTTGTTTTTCTCACTTGTTATTATCAAACCTAATGGTTTTTAATTTTGGTACA
ACTCCTTAAAGGGTTGAAGGTTGTGACAATAACTGAGGGAACTGATGTTCTGAATAAATGATGTGA
AGTAAACACAATTGTATTTGAAAAAAAAAAAAAAAAAAAAAAAAAAA
Homo sapiens IRAK1BP1 3′-UTR
NM_001010844
(SEQ ID NO: 267)
AATTCCAAACAAATTATATTGTACTTGTATCTTTTTACCTATTTTTATACTTTTTATAATGTTTAC
GTTTGTCCTGAATATATA
Homo sapiens CDKL2 3′-UTR
CDKL2-002 ENST00000307465
(SEQ ID NO: 268)
GAACCATTTTGGTTCTGAACTGGATGATGCTCTTGCACTTGAGATGACATCTTCTTGCAGCAAGAG
TGCTGATATCCCAAGAGGAGAGATTCATGGTTTTGATCATTTCCTTCTGAACTGCCTGCATTTTCT
GAGGAAGGCCTTCTAGAAGAAGGAAAGACAAAGACTTCCAAATGTTTCAAAGGAAGATTGAACAAA
TGGCCCTCCCCAACTGTTATCCCATTACCTTTCACGTCCACCGATGCTATTTCAAGACATATCCAG
TGGAATAACAGTGATATGGTTCTTGTTACATGAATGTGTATTTACTGTTAGGAGATTGTATATTTT
AAGTTACC
Homo sapiens GHR 3′-UTR
GHR-202 ENST00000537449
(SEQ ID NO: 269)
CCTTTCTTTGGTTTCCCAAGAGCTACGTATTTAATAGCAAAGAATTGACTGGGGCAATAACGTTTA
AGCCAAAACAATGTTTAAACCTTTTTTGGGGGAGTGACAGGATGGGGTATGGATTCTAAAATGCCT
TTTCCCAAAATGTTGAAATATGATGTTAAAAAAATAAGAAGAATGCTTAATCAGATAGATATTCCT
ATTGTGCAATGTAAATATTTTAAAGAATTGTGTCAGACTGTTTAGTAGCAGTGATTGTCTTAATAT
TGTGGGTGTTAATTTTTGATACTAAGCATTGAATGGCTATGTTTTTAATGTATAGTAAATCACGCT
TTTTGAAAAAGCGAAAAAATCAGGTGGCTTTTGCGGT
Homo sapiens KIAA1107 3′-UTR
NM_015237
(SEQ ID NO: 270)
GTGTTAACATTTTGGAAAAATTTATGCCACTCCTTTATTTTTTGATGCCTATATTATATCCAAATG
ATAATTGCATTAGCCGGATATAAACTTTCTTTAATATTGAGTCTTTCCAATTTAATGAGGTAAACA
TAGTTTATTTATTAATATATCACATATAGAAAAATGTTTTTCTAAAGTTTTTGAGCATGTTTTCTC
TAATTATTAGAGAAATTAGAAGACTTATAAGGAAACCCTAGCTTCAGTTTTCCTTTCCTAGCTGAT
GATTTGTTCACTTAATCATTATTCAAGAATTTAAAATGTGAATGCAGAAGTAGATCAGTCCCTTTA
CTTTTTGCTCTGCATAGGGTAACATAGTAATTTAACAATAAAAACTTACCGTGCTTGTGTCCAAAA
AAAAAAAAAA
Homo sapiens RPS6KA6 3′-UTR
RPS6KA6-001 ENST00000262752
(SEQ ID NO: 271)
GATTTGTGGTGTTCCTAGGCCAAACTGGATGAAGATGAAATTAAATGTGTGGCTTTTTTCCTATTC
TTATCAAAGGCATCGTTGTCTGCTAAATTACTTGAATATTAAGTAATATTAAATCCCCATTTTTAG
GGGAAGTGAGATTTAAAAAACCATTCACAGGTCCACAATATTCATACTATGTGTTTGCAGTAGTGT
TCAAGTGTTTATTTAAGCATATAATTGGTGTCCACCAGGTCCTCACAACTTCTCTGCACACAAGCT
TCTAAAATTCCTTTCAAATAAAGTTACTTTAATATTT
Homo sapiens CLGN 3′-UTR
NM_004362, NM_001130675
(SEQ ID NO: 272)
ACTAGATTGAAATATTTTTAATTCCCGAGAGGGATGTTTGGCATTGTAAAAATCAGCATGCCAGAC
CTGAACTTTAATCAGTCTGCACATCCTGTTTCTAATATCTAGCAACATTATATTCTTTCAGACATT
TATTTTAGTCCTTCATTTCAGAGGAAAAAGAAGCAACTTTGAAGTTACCTCATCTTTGAATTTAGA
ATAAAAGTGGCACATTACATATCGGATCTAAGAGATTAATACCATTAGAAGTTACACAGTTTTAGT
TGTTTGGAGATAGTTTTGGTTTGTACAGAACAAAATAATATGTAGCAGCTTCATTGCTATTGGAAA
AATCAGTTATTGGAATTTCCACTTAAATGGCTATACAACAATATAACTGGTAGTTCTATAATAAAA
ATGAGCATATGTTCTGTTGTGAAGAGCTAAATGCAATAAAGTTTCTGTATGGTTGTTTGATTCTAT
CAACAATTGAAAGTGTTGTATATGACCCACATTTACCTAGTTTGTGTCAAATTATAGTTACAGTGA
GTTGTTTGCTTAAATTATAGATTCCTTTAAGGACATGCCTTGTTCATAAAATCACTGGATTATATT
GCAGCATATTTTACATTTGAATACAAGGATAATGGGTTTTATCAAAACAAAATGATGTACAGATTT
TTTTTCAAGTTTTTATAGTTGCTTTATGCCAGAGTGGTTTACCCCATTCACAAAATTTCTTATGCA
TACATTGCTATTGAAAATAAAATTTAAATATTTTTTCATCCTGAAAAAAAA
Homo sapiens CLGN-202 3′-UTR
NM_004362, NM_001130675
ENST00000325617
(SEQ ID NO: 273)
ACTAGATTGAAATATTTTTAATTCCCGAGAGGGATGTTTGGCATTGTAAAAATCAGCATGCCAGAC
CTGAACTTTAATCAGTCTGCACATCCTGTTTCTAATATCTAGCAACATTATATTCTTTCAGACATT
TATTTTAGTCCTTCATTTCAGAGGAAAAAGAAGCAACTTTGAAGTTACCTCATCTTTGAATTTAGA
ATAAAAGTGGCACATTACATATCGGATCTAAGAGATTAATACCATTAGAAGTTACACAGTTTTAGT
TGTTTGGAGATAGTTTTGGTTTGTACAGAACAAAATAATATGTAGCAGCTTCATTGCTATTGGAAA
AATCAGTTATTGGAATTTCCACTTAAATGGCTATACAACAATATAACTGGTAGTTCTATAATAAAA
ATGAGCATATGTTCTGTTGTGAAGAGCTAAATGCAATAAAGTTTCTGTATGGTTGTTTGATTCTAT
CAAC
Homo sapiens TMEM45A 3′-UTR
NM_018004
(SEQ ID NO: 274)
CTTTGATGAGCTTCCAGTTTTTCTAGATAAACCTTTTCTTTTTTACATTGTTCTTGGTTTTGTTTC
TCGATCTTTTGTTTGGAGAACAGCTGGCTAAGGATGACTCTAAGTGTACTGTTTGCATTTCCAATT
TGGTTAAAGTATTTGAATTTAAATATTTTCTTTTTAGCTTTGAAAATATTTTGGGTGATACTTTCA
TTTTGCACATCATGCACATCATGGTATTCAGGGGCTAGAGTGATTTTTTTCCAGATTATCTAAAGT
TGGATGCCCACACTATGAAAGAAATATTTGTTTTATTTGCCTTATAGATATGCTCAAGGTTACTGG
GCTTGCTACTATTTGTAACTCCTTGACCATGGAATTATACTTGTTTATCTTGTTGCTGCAATGAGA
AATAAATGAATGTATGTATTTTGGTGC
Homo sapiens TBC1D8B 3′-UTR
TBC1D8B-007 ENST00000276175
(SEQ ID NO: 275)
ATCCCTAGGAATTGCCTATCATAGACAAGTTTACTAACATTCCTGTAGCTGTCAGTTTGATTCCTG
TGAGTAGGGCTCAGGGATTTATCTTGTTACCAATGTGTCTGAAGGCCAAAATATATATCCAGAAGC
ACAATGCATCATTCCTTTGT
Homo sapiens ACP6 3′-UTR
NM_016361
(SEQ ID NO: 276)
CTGATTTATAAAAGCAGGATGTGTTGATTTTAAAATAAAGTGCCTTTATACAATGCCAAAAAAAAA
AAAAAAAAAAAAAAA
Homo sapiens RP6-213H19.1 3′-UTR
MST4-003 (RBM4B-003 ENST00000496850)
(SEQ ID NO: 277)
GAAACTTATTATTGGCTTCTGTTTCATATGGACCCAGAGAGCCCCACCAAACCTACGTCAAGATTA
ACAATGCTTAACCCATGAGCTCCATGTGCCTTTTGGATCTTTGCA
Homo sapiens SNRPN 3′-UTR
NM_022807
(SEQ ID NO: 278)
CATACTGTTGATCCATCTCAGTCACTTTTTCCCCTGCAATGCGTCTTGTGAAATTGTGTAGAGTGT
TTGTGAGCTTTTTGTTCCCTCATTCTGCATTAATAATAGCTAATAATAAATGCATAGAGCAATTAA
ACTGTG
Homo sapiens GLRB 3′-UTR
GLRB-005 ENST00000512619
(SEQ ID NO: 279)
GATCTAATGACTTCAGCATTGTTGGAAGCTTACCAAGAGATTTTGAACTATCCAATTATGACTGCT
ATGGAAAACCCATTGAAGTTAACAACGGACTTGGGAAATCTCAGGCTAAGAACAACAAGAAGCCTC
CCCCTGCGAAACCTGTTATTCCAACAGCAGCAAAGCGAATTGATCTTTATGCAAGAGCATTGTTTC
CTTTCTGCTTCTTGTTCTTCAATGTTATATATTGGTCTATATATTTATGATAAATCTTTTCCATTT
GTACAAAATAAAATTCCATTTCATTGTGACCTACTCCTTTCATAAATGCCAATCTGTGAGAACTTT
TGAATTTTCATAGCAACATTGCATTTTGGATGCCATTTGATTGTAATAAAACTGTGGCACCTTAAT
TTTGAATGGCAGCATGATCATGTAATATC
Homo sapiens HERC6 3′-UTR
NM_017912
(SEQ ID NO: 280)
TCACCTCTGAGAGACTCAGGGTGGGCTTTCTCACACTTGGATCCTTCTGTTCTTCCTTACACCTAA
ATAATACAAGAGATTAATGAATAGTGGTTAGAAGTAGTTGAGGGAGAGATTGGGGGAATGGGGAGA
TGATGATGATGGTCAAAGGGTGCAAAATCTCACACAAGACTGAGGCAGGAGAATAGGGTACAGAGA
TAGGGATCTAAGGATGACTTGGACACACTCCCTGGCACTGAAGAGTCTGAACACTGGCCTGTGATT
GGTCCATTCCAGGACCTTCATTTGCATAAGGTATCAAACCACATCAGCCTCTGATTGGCCATGGGC
CAGACCTGCACTCTGGCCAATGATTGGTTCATTCCAGGACATTCATTTGCATAAGGAGTCAAACCA
CACCAGTCTTGGATTGGCTGTGAGCCAATTCACCTCAGTCTCTAATTGGCTGTGAGTCAGTCTTTC
ATTTACATAGGGTGTAACCATCAAGAAACCTCTACAGGGTACTTAAGCCCCAGAAGATTTTGCTAC
CAGGGCTCTTGAGCCACTTGCTCTAGCCCACTCCCACCCTGTGGAATGTACTTTCACTTTTGCTGC
TTCACTGCCTTGTGCTCCAATAAATCCACTCCTTCACCACCCAAAAAAAAAAAAAAA
Homo sapiens CFH 3′-UTR
NM_000186
(SEQ ID NO: 281)
AATCAATCATAAAGTGCACACCTTTATTCAGAACTTTAGTATTAAATCAGTTCTCAATTTCATTTT
TTATGTATTGTTTTACTCCTTTTTATTCATACGTAAAATTTTGGATTAATTTGTGAAAATGTAATT
ATAAGCTGAGACCGGTGGCTCTCTTCTTAAAAGCACCATATTAAATCCTGGAAAACTAAAAAAAAA
AAAAAAAAAAAAAAAAAAAAAAAAAAAAAAAAAAAAAAAAAAAAAAAAAAAAAAAAAAAAAAAAAA
Homo sapiens GALC 3′-UTR
GALC-002 ENST00000393569
(SEQ ID NO: 282)
TACTTAACAGGGCATCATAGAATACTCTGGATTTTCTTCCCTTCTTTTTGGTTTTGGTTCAGAGCC
AATTCTTGTTTCATTGGAACAGTATATGAGGCTTTTGAGACTAAAAATAATGAAGAGTAAAAGGGG
AGAGAAATTTATTTTTAATTTACCCTGTGGAAGATTTTATTAGAATTAATTCCAAGGGGAAAACTG
GTGAATCTTTAACATTACCTGGTGTGTTCCCTAACATTCAAACTGTGCATTGGCCATACCCTTAGG
AGTGGTTTGAGTAGTACAGACCTCGAAGCCTTGCTGCTAACACTGAGGTAGCTCTCTTCATCTTAT
TTGCAAGCGGTCCTGTAGATGGCAGTAACTTGATCATCACTGAGATGTATTTATGCATGCTGACCG
TGTGT
Homo sapiens GALC 3′-UTR
GALC-005 ENST00000393568
(SEQ ID NO: 283)
TACTTAACAGGGCATCATAGAATACTCTGGATTTTCTTCCCTTCTTTTTGG
Homo sapiens PDE1A 3′-UTR
NM_001003683.2
(SEQ ID NO: 284)
ACACCTTTAAGTAAAACCTCGTGCATGGTGGCAGCTCTAATTTGACCAAAAGACTTGGAGATTTTG
ATTATGCTTGCTGGAAATCTACCCTGTCCTGTGTGAGACAGGAAATCTATTTTTGCAGATTGCTCA
ATAAGCATCATGAGCCACATAAATAACAGCTGTAAACTCCTTAATTCACCGGGCTCAACTGCTACC
GAACAGATTCATCTAGTGGCTACATCAGCACCTTGTGCTTTCAGATATCTGTTTCAATGGCATTTT
GTGGCATTTGTCTTTACCGAGTGCCAATAAATTTTCTTTGAGCAGCTAATTGCTAATTTTGTCATT
TCTACAATAAAGCTTGGTCCACCTGTTTTC
Homo sapiens PDE1A 3′-UTR
PDE1A-003 ENST00000410103
(SEQ ID NO: 285)
ACACCTTTAAGTAAAACCTCGTGCATGGTGGCAGCTCTAATTTGACCAAAAGACTTGGAGATTTTG
ATTATGCTTGCTGGAAATCTACCCTGTCCTGTGTGAGACAGGAAATCTATTTTTGCAGATTGCTCA
ATAAGCATCATGAGCCACATAAATAACAGCTGTAAACTCCTTAATTCACCGGGCTCAACTGCTACC
GAACAGATTCATCTAGTGGCTACATCAGCACCTTGTGCTTTCAGATATCTGTTTCAATGGCATTTT
GTGGCATTTGTCTTTACCGAGTGCCAATAAATTTTCTTTGAGCA
Homo sapiens GSTM5 3′-UTR
NM_000851
(SEQ ID NO: 286)
GGCCCAGTGATGCCAGAAGATGGGAGGGAGGAGCCAACCTTGCTGCCTGCGACCCTGGAGGACAGC
CTGACTCCCTGGACCTGCCTTCTTCCTTTTTCCTTCTTTCTACTCTCTTCTCTTCCCCAAGGCCTC
ATTGGCTTCCTTTCTTCTAACATCATCCCTCCCCGCATCGAGGCTCTTTAAAGCTTCAGCTCCCCA
CTGTCCTCCATCAAAGTCCCCCTCCTAACGTCTTCCTTTCCCTGCACTAACGCCAACCTGACTGCT
TTTCCTGTCAGTGCTTTTCTCTTCTTTGAGAAGCCAGACTGATCTCTGAGCTCCCTAGCACTGTCC
TCAAAGACCATCTGTATGCCCTGCTCCCTTTGCTGGGTCCCTACCCCAGCTCCGTGTGATGCCCAG
TAAAGCCTGAACCATGCCTGCCATGTCTTGTCTTATTCCCTGAGGCTCCCTTGACTCAGGACTGTG
CTCGAATTGTGGGTGGTTTTTTGTCTTCTGTTGTCCACAGCCAGAGCTTAGTGGATGGGTGTGTGT
GTGTGTGTGTTGGGGGTGGTGATCAGGCAGGTTCATAAATTTCCTTGGTCATTTCTGCCCTCTAGC
CACATCCCTCTGTTCCTCACTGTGGGGATTACTACAGAAAGGTGCTCTGTGCCAAGTTCCTCACTC
ATTCGCGCTCCTGTAGGCCGTCTAGAACTGGCATGGTTCAAAGAGGGGCTAGGCTGATGGGGAAGG
GGGCTGAGCAGCTCCCAGGCAGACTGCCTTCTTTCACCCTGTCCTGATAGACTTCCCTGATCTAGA
TATCCTTCGTCATGACACTTCTCAATAAAACGTATCCCACCGTATTGTAAAAAAAAAAAAAAA
Homo sapiens CADPS2 3′-UTR
CADPS2-002 ENST00000412584
(SEQ ID NO: 287)
TATCACACAGCTTTGCAGAAGGAAGGAAGACCTTGATCGACATTGTTTTTTATTTTTTTAACCTTG
TCCTTGTAATTACATTCATTGTTTGTTTTGGCCAAATAAAAATGCTTGTATTTCTTTAAAAAGTAA
GCCTGAATGTAGAGTAAAAGGGGAAATGCCAAGATTTTGGGGTTTTTTTGTTTCCTTTTTTTGTTT
GTTTGTTTGTTTGTTTTTTTGGAGAAGAGCATCCTCTTTTGTGTAGTTTGACCTAAAAATGAACCT
TGGCTCTGCTTGTGATCAGAACATGAACTTTTTTTTTTAAAGAAGATTTGAGCATTTTTCTGTAAT
CACATCAAAATGATGTTTTCTGTGTAAAGCGAGATACATATTTCTCATAATGCAGCATTGTGAGAA
GTCAGTTCGGACCACTGCACCAA
Homo sapiens CADPS2 3′-UTR
CADPS2-001 ENST00000449022
(SEQ ID NO: 288)
TATCACACAGCTTTGCAGAAGGAAGGAAGACCTTGATCGACATTGTTTTTTATTTTTTTAACCTTG
TCCTTGTAATTACATTCATTGTTTGTTTTGGCCAAATAAAAATGCTTGTATTTCTTTAAAAAGTAA
GCCTGAATGTAGAGTAAAAGGGGAAATGCC
Homo sapiens AASS 3′-UTR
AASS-001 ENST00000417368
(SEQ ID NO: 289)
TTGGGAATTATATTTTGTTTTTTTCTTCCCAGGCAATACACCTCTGAACATGTGTGTGATAAATGG
GTTTGCTAATGTGCTGTTTTAAAGTATAAAGCATAATATGTTTTGGTTAACACAATGTACTTTTTG
AACTATAAATCTTTATTTTAATATGGAAATGTTTGGAACAGGAGATGCAAGCCACTAACAGAGAAC
TTTAATAATTCTACCCTGTATTTTATAAATACGTATGTGAAAGTGATGA
Homo sapiens TRIM6-TRIM34 3′-UTR
NM_001003819
(SEQ ID NO: 290)
ATTTTCTCATTTCTTCACCTACAACCCTTTGTCTTGACTTATCTCCTGCAACTGACTCATCTGCAA
CATTCACACCATTGCTTCCTTGTGGTTTCCCTTCTTTAGAACTTTTACTCATCCTTGAGATGTATG
GTGTATTTGGCTTGAGTTATGAGAGATGCTTATTTATTCATTTACTCTTTTTCATATTTTCAGAGA
AAGTTACCTAATCCCTCCTAAAGACACAGCAGTATGGGTATAACATCCTTGCCTTCCCATTTATCC
ATGTTTCACTTTATCACTGATATGAAGAGGCCCAAAGCCTGTTAGCCACCATCCATGCTACCTAGG
TAGTCCATAGGAACCACCCCCATGACCACCACCAACATCAACTAAAGGTTCTTGGAGGGTATGTCA
GTGTGTTGCTCAGGATACCCCAGGTACATCAAGGAATCAAGGAGAGGAAAATATGAGCAATATGTG
TATTCAGAGTGAAGATTTTATGTCCAGAGTATTTGAGCTCAAACCTTGCCTGTTGTTTTCTAATCA
TGATGAATACTTTCTCAGTTTCTTTTTCCTGAAATATAAATTGGGATTTAAGACTGTACCTAACTA
TTAAGATCACTGTGTAAAACTAAGTGTCTCTAAATGTAATGCATCGATTTAGTGTCTGGAACATAA
TAAATATTTGCTCTCATGATTGCTAAAAAAAAAAA
Homo sapiens SEPP1 3′-UTR
NM_005410
(SEQ ID NO: 291)
ATATTTAAAATAGGACATACTCCCCAATTTAGTCTAGACACAATTTCATTTCCAGCATTTTTATAA
ACTACCAAATTAGTGAACCAAAAATAGAAATTAGATTTGTGCAAACATGGAGAAATCTACTGAATT
GGCTTCCAGATTTTAAATTTTATGTCATAGAAATATTGACTCAAACCATATTTTTTATGATGGAGC
AACTGAAAGGTGATTGCAGCTTTTGGTTAATATGTCTTTTTTTTTCTTTTTCCAGTGTTCTATTTG
CTTTAATGAGAATAGAAACGTAAACTATGACCTAGGGGTTTCTGTTGGATAATTAGCAGTTTAGAA
TGGAGGAAGAACAACAAAGACATGCTTTCCATTTTTTTCTTTACTTATCTCTCAAAACAATATTAC
TTTGTCTTTTCAATCTTCTACTTTTAACTAATAAAATAAGTGGATTTTGTATTTTAAGATCCAGAA
ATACTTAACACGTGAATATTTTGCTAAAAAAGCATATATAACTATTTTAAATATCCATTTATCTTT
TGTATATCTAAGACTCATCCTGATTTTTACTATCACACATGAATAAAGCCTTTGTATCTTTCTTTC
TCTAATGTTGTATCATACTCTTCTAAAACTTGAGTGGCTGTCTTAAAAGATATAAGGGGAAAGATA
ATATTGTCTGTCTCTATATTGCTTAGTAAGTATTTCCATAGTCAATGATGGTTTAATAGGTAAACC
AAACCCTATAAACCTGACCTCCTTTATGGTTAATACTATTAAGCAAGAATGCAGTACAGAATTGGA
TACAGTACGGATTTGTCCAAATAAATTCAATAAAAACCTTAAAGCTGAAAAAAAAAAAAAAAAAAA
AAAAAAAAAAAAAAAAAAAAAAAAAAAAAAAAAAAAAAAAAAAAAAAAAAAAAAAAAAAA
Homo sapiens SEPP1 3′-UTR
SEPP1-004 ENST00000506577
(SEQ ID NO: 292)
ATATTTAAAATAGGACATACTCCCCAATTTAGTCTAGACACAATTTCATTTCCAGCATTTTTATAA
ACTACCAAATTAGTGAACCAAAAATAGAAATTAGATTTGTGCAAACATGGAGAAATCTACTGAATT
GGCTTCCAGATTTTAAATTTTATGTCATAGAAATATTGACTCAAACCATATTTTTTATGATGGAGC
AACTGAAAGGTGATTGCAGCTTTTGGTTAATATGTCTTTTTTTTTCTTTTTCCAGTGTTCTATTTG
CTTTAATGAGAATAGAAACGTAAACTATGACCTAGGGGTTTCTGTTGGATAATTAGCAGTTTAGAA
TGGAGGAAGAACAACAAAGACATGCTTTCCATTTTTTTCTTTACTTATCTCTCAAAACAATATTAC
TTTGTCTTTTCAATCTTCTACTTTTAACTAATAAAATAAGTGGATTTTGTATTTTAAGATCCAGAA
ATACTTAACACGTGAATATTTTGCTAAAAAAGCATATATAACTATTTTAAATATCCATTTATCTTT
TGTATATCTAAGACTCATCCTGATTTTTACTATCACACATGAATAAAGCCTTTGTATCTTT
Homo sapiens PDE5A 3′-UTR
PDE5A-002 ENST00000264805
(SEQ ID NO: 293)
GTGGCCTATTTCATGCAGAGTTGAAGTTTACAGAGATGGTGTGTTCTGCAATATGCCTAG
Homo sapiens SATB1 3′-UTR
SATB1-004 ENST00000417717
(SEQ ID NO: 294)
GATAAAAGTATTTGTTTCGTTCAACAGTGCCACTGGTATTTACTAACAAAATGAAAAGTCCACCTT
GTCTTCTCTCAGAAAACCTTTGTTGTTCATTGTTTGGCCAATGAATCTTCAAAAACTTGCACAAAC
AGAAAAGTTGGAAAAGGATAATACAGACTGCACTAAATGTTTTCCTCTGTTTTACAAACTGCTTGG
CAGCCCCAGGTGAAGCATCAAGGATTGTTTGGTATTAAAATTTGTGTTCACGGGATGCACCAAAGT
GTGTACCCCGTAAGCATGAAACCAGTGTTTTTTGTTTTTTTTTTAGTTCTTATTCCGGAGCCTCAA
ACAAGCATTATACCTTCTGTGATTATGATTTCCTCTCCTATAATTATTTCTGTAGCACTCCACACT
GATCTTTGGAAACTTGCCCCTTATTT
Homo sapiens CCPG1 3′-UTR
CCPG1-002 ENST00000442196
(SEQ ID NO: 295)
TTCACAATTGAGTTAAATTAGACAACTGTAAGAGAAAAATTTATGCTTTGTATAATGTTTGGTATT
GAAACTAATGAAATTACCAAGATGACAATGTCTTTTCTTTTGTTTCTAAGTATCAGTTTGATAACT
TTATATTATTCCTCAGAAGCATTAGTTAAAAGTCTACTAACCTGCATTTTCCTGTAGTTTAGCTTC
GTTGAATTTTTTTTGACACTGGAAATGTTCAACTGTAGTTTTATTAAGGAAGCCAGGCATGCAACA
GATTTTGTGCATGAAATGAGACTTCCTTTCAGTGTAAGAGCTTAAAGCAAGCTCAGTCATACATGA
CAAAGTGTAATTAACACTGATGTTTGTGTTAAATTTGCAGCAGAGCTTGAGAAAAGTACATTGTTC
TGGAATTTCATCATTAACATTTTATAATCTTACACTCACTTCTTGTCTTTTTGTGGGTTCAAGAGC
CCTCTGACTTGTGAAGAATTTGCTGCCCTCTTAAGAGCTTGCTGACTTGTTTTCTTGTGAAATTTT
TTGCACATCTGAATATCGTGGAAGAAACAATAAAACTACACCATGAGGAAAACTAAAGGTCTTTAT
TTAAAATCTGGCATTGTATTAACATGTAATTTTATACTATGTGGTATTTTATACATTTCCTCAGTA
GTGATATTTGGTAAAGCAGTTCATACAGCTTTTTTCTAAGTTCCATGAATCTTACCCAGTGTTTAC
CGAAGTATTTAAGCAGCATCTGAATATTTCCACCCAGCAATGTTAATTTATCTAGGAAAGTTCAGA
ATTTCATCTTCATGTTGAATTTCCCTTTTAACTTCCGTTCATAGACATATATGTGACTTCCAATTC
GACCCTCTGGCAAGTGAGTGTGGAAGAAAACAGCAGTTCTTTTATAATTGCTTGAAATTAGGAAAG
CGCTTATTTCCTAGAAGCAAATAAATGTTTAAGTAAATAAAGGCTACATTTTGCTGA
Homo sapiens CCPG1 3′-UTR
CCPG1-004 ENST00000425574
(SEQ ID NO: 296)
TTCACAATTGAGTTAAATTAGACAACTGTAAGAGAAAAATTTATGCTTTGTATAATGTTTGGTATT
GAAACTAATGAAATTACCAAGATGACAATGTCTTTTCTTTTGTTTCTAAGTATCAGTTTGATAACT
TTATATTATTCCTCAGAAGCATTAGTTAAAAGTCTACTAACCTGCATTTTCCTGTAGTTTAGCTTC
GTTGAATTTTTTTTGACACTGGAAATGTTCAACTGTAGTTTTATTAAGGAAGCCAGGCATGCAACA
GATTTTGTGCATGAAATGAGACTTCCTTTCAGTGTAAGAGCTTAAAGCAAGCTCAGTCATACATGA
CAAAGTGTAATTAACACTGATGTTTGTGTTAAATTTGCAGCAGAGCTTGAGAAAAGTACATTGTTC
TGGAATTTCATCATTAACATTTTATAATCTTACACTCACTTCTTGTCTTTTTGTGGGTTCAAGAGC
CCTCTGACTTGTGAAGAATTTGCTGCCCTCTTAAGAGCTTGCTGACTTGTTTTCTTGTGAAATTTT
TTGCACATCTGAATATCGTGGAAGAAACAATAAAACTACACCATGAG
Homo sapiens CNTN1 3′-UTR
CNTN1-002 ENST00000348761
(SEQ ID NO: 297)
ATGTGTTGTGACAGCTGCTGTTCCCATCCCAGCTCAGAAGACACCCTTCAACCCTGGGATGACCAC
AATTCCTTCCAATTTCTGCGGCTCCATCCTAAGCCAAATAAATTATACTTTAACAAACTATTCAAC
TGATTTACAACACACATGATGACTGAGGCATTCGGGAACCCCTTCATCCAAAAGAATAAACTTTTA
AATGGATATAAATGATTTTTAACTCGTTCCAA
Homo sapiens CNTN1 3′-UTR
CNTN1-004 ENST00000547849
(SEQ ID NO: 298)
TCGTTGACACTCACCATTTCTGTGAAAGACTTTTTTTTTTTTAACATATTATACTAGATTTGACTA
ACTCAATCTTGTAGCTTCTGCAGTTCTCCCCACCCCCAACCTAGTTCTTAGAGTATGTTTCCCCTT
TTGAAACATGTAAACATACTTTGGGCATAAATATTTTTTAAAATATAACTATAATGCTTCACTAAT
ACCTTAAAAATGCCTAGTGAACTAACTCAGTACATTATATAATGGCCAAGTGAAAGTTTTGTGTTT
TCATGTCCTGTTTTTCTTTGAAATTATATAGCCCAGAAATTAGCTCATTATCTGAAAAACGTATGA
AGAACTGATGAATTGTATAATACAGGAGTATTGCCATTGAATGTACTGTTTGATTTATTCAAGCAG
GTAATGAACAATGTTGTCAAACTCTCTAATGAGACATCATAATTAGGACATAAGCTAAAAGGGGCA
TTACTCCGGCAGTCTTTTTTTCTTAATCCTAGTACCATACATATTCTTTGGCATGAAAGAATGAAA
AGCATTAGTAAACAACTGAAGTCCTACCATGGCTCTGTAGGGTTTTTGGAACAATTCCTGGAATTG
GAAAGTGAAAATGGATAGCATGTGGGGGAAACCCTCATCTGAGTAGCAAGATTTTAGTAAAGATGA
CTAAGCCATTAACAGCATGCATTCATATTTAATTTTATTGACTCCTGCCATCAGCTTTTGTAGATC
GTTTGGGTGGAAGGTTGTGATTTTTACTGGGAGGACTTGAGTAGAAGTGGATGATTAAAATTGAGG
AGTATATAATTCTTTCTGGGACTGCTTAAATGTTATTGTTTGAAAATACCTTCACTTTCCCCCTTT
GGTCAAAGAGATGTGCTTAAAATTCTTATTCCTTCACAATAAATAATTTTGATTTTCTTAGACA
Homo sapiens CNTN1 3′-UTR
CNTN1-004 ENST00000547849
+T at pos. 30bp, mutations G727bpT, A840bpG
(SEQ ID NO: 299)
TTTTTTCGTTGACACTCACCATTTCTGTGAA
AGACTTTTTTTTTTTTTAACATATTATACTAGATTTGACTAACTCAATCTTGTAGCTTCT
GCAGTTCTCCCCACCCCCAACCTAGTTCTTAGAGTATGTTTCCCCTTTTGAAACATGTAA
ACATACTTTGGGCATAAATATTTTTTAAAATATAACTATAATGCTTCACTAATACCTTAA
AAATGCCTAGTGAACTAACTCAGTACATTATATAATGGCCAAGTGAAAGTTTTGTGTTTT
CATGTCCTGTTTTTCTTTGAAATTATATAGCCCAGAAATTAGCTCATTATCTGAAAAACG
TATGAAGAACTGATGAATTGTATAATACAGGAGTATTGCCATTGAATGTACTGTTTGATT
TATTCAAGCAGGTAATGAACAATGTTGTCAAACTCTCTAATGAGACATCATAATTAGGAC
ATAAGCTAAAAGGGGCATTACTCCGGCAGTCTTTTTTTCTTAATCCTAGTACCATACATA
TTCTTTGGCATGAAAGAATGAAAAGCATTAGTAAACAACTGAAGTCCTACCATGGCTCTG
TAGGGTTTTTGGAACAATTCCTGGAATTGGAAAGTGAAAATGGATAGCATGTGGGGGAAA
CCCTCATCTGAGTAGCAAGATTTTAGTAAAGATGACTAAGCCATTAACAGCATGCATTCA
TATTTAATTTTATTGACTCCTGCCATCAGCTTTTGTAGATCTTTTGGGTGGAAGGTTGTG
ATTTTTACTGGGAGGACTTGAGTAGAAGTGGATGATTAAAATTGAGGAGTATATAATTCT
TTCTGGGACTGCTTAAATGTTATTGTTTGAAAATGCCTTCACTTTCCCCCTTTGGTCAAA
GAGATGTGCTTAAAATTCTTATTCCTTCACAATAAATAATTTTGATTTTCTTAGACA
Homo sapiens LMBRD2 3′-UTR
(SEQ ID NO: 300)
AGTCTGAAAAAGTTTGTGGGACCACTAACCAAGGTCAACACATCAGTTCAGTCTTGATGAACATCT
GTGTACCCTAGAATTTCCTCTATACACAGTGAAAAGTGTCAAGATAACAAAAAAGGCACTGAGAAT
TAATTATATCTTAGGAATAATAGTTTAATGTGCATTGAATAGAGTATCACCTTTTTCAACAAGATT
TATTACATATCATTTCCTAAGCATCTGCCTTAGAAATACAGTTACAGTGGAAGGACTTTAAGAAAG
ATCAACATATGTTAAGAACATGCAGTTCAGTTTGTTTCAGATTAATTTTTTTTCAAGAGAGTTATT
TTAAAGATTCAAGGAAGCCATAAGTCATACTAAATAATATTATATACAGTTTTGTTATTGTGACTT
ACATTTTTGTTACTTCTAAAAAGTATATTCAACCTGTATTTCCCAAAGAAATGTAAGTGAATGGAG
ACCTCAAATAATAACTGTATTCATAAAACTCGTGTCTTAAAACAAGGCTTACTTACTAGACATAAC
TGAATGTAAAAAGTGCTTTTTCAAATCTGTTTGCAAACTCGTGGGGGATTTTTGCATGTATAAGAT
TAAGATTATACTTCAAGTGATGCGTGTCTGTGTATTTAGCATGTGTACTATAATCAGGTGATATAG
TATTCCTTCAGTCTTTGTAGTAACTGGATTTTTTTATGCTTCTGGTATTGCTTTATAAAAGATTTT
CATTTCAG
Homo sapiens TLR3 3′-UTR
NM_003265
(SEQ ID NO: 301)
ATTTATTTAAATATTCAATTAGCAAAGGAGAAACTTTCTCAATTTAAAAAGTTCTATGGCAAATTT
AAGTTTTCCATAAAGGTGTTATAATTTGTTTATTCATATTTGTAAATGATTATATTCTATCACAAT
TACATCTCTTCTAGGAAAATGTGTCTCCTTATTTCAGGCCTATTTTTGACAATTGACTTAATTTTA
CCCAAAATAAAACATATAAGCACGTAAAAAAAAAAAAAAAAAA
Homo sapiens BCAT1 3′-UTR
BCAT1-002 ENST00000342945
(SEQ ID NO: 302)
ATGGAAAATAGAGGATACAATGGAAAATAGAGGATACCAACTGTATGCTACTGGGACAGACTGTTG
CATTTGAATTGTGATAGATTTCTTTGGCTACCTGTGCATAATGTAGTTTGTAGTATCAATGTGTTA
CAAGAGTGATTGTTTCTTCATGCCAGAGAAAATGAATTGCAATCATCAAATGGTGTTTCATAACTT
GGTAGTAGTAACTTACCTTACCTTACCTAGAAAAACATTAATGTAAGCCATATAACATGGGATTTT
CCTCAATGATTTTAGTGCCTCCTTTTGTACTTCACTCAGATACTAAATAGTAGTTTATTCTTTAAT
ATAAGTTACATTCTGCTCCTCAAACAAATGCAATTTTTTGTGTGTGTTTGAAAGCTAATTTGAGAA
AATTTCATAGGTTACATTTCCTGCAGCCTATCTTTATCCACAGAAAGTGTTTTCTTTTTTTTAAAT
CAAGACTTTTAAAACTGGATTTCCTCCCATCACTGTTTTTTGAAGGTCCTCCAAGTCCGTGTTAA
Homo sapiens BCAT1 3′-UTR
(SEQ ID NO: 303)
ATGGAAAATAGAGGATACAATGGAAAATAGAGGATACCAACTGTATGCTACTGGGACAGACTGTTG
CATTTGAATTGTGATAGATTTCTTTGGCTACCTGTGCATAATGTAGTTTGTAGTATCAATGTGTTA
CAAGAGTGATTGTTTCTTCATGCCAGAGAAAATGAATTGCAATCATCAAATGGTGTTTCATAACTT
G
Homo sapiens TOM1L1 3′-UTR
TOM1L1-001 ENST00000575882
(SEQ ID NO: 304)
GAAGAAAGTGGATGATCAGCTCACTACCACATCAAAGGTGCCAACTCTCTAAAACGTAGACTCTGT
GCAGCTTTGAAGCCTGGAAGACAATACCTACCAACATGTCAAAGCCATGGTGGCACATTTCTGCTA
TAATGAAGATTAAATAGAATAACAGTTCCAGGATAACACTGATTCCTGACAACAGCGTGAGATTTC
AACAGAACTTGTTTGGAACAAATACTCACTTAAAACTTCAGCAGAAGAAAAATTACTTAGTCCTTA
GGCCAACCAATTTAACTGCAGTGTCATGTTTCACAGGCCTTCCTACATTTAGAAATCGTCACACAG
CTGTGATAAGAGTAGATTATTTTACTATGAAATAATTCTGAATAGATGAAAGCATAAAATGTGAGA
AACTGAATGTATTATTCAGGAAGAATACTGAGTGCCTTCATTTAACTAAAGTTGAATGTAAAAGTC
AATTTGCACTTCTTTATAATCCTCTGGTTTAGAATTATAAATTGTTAAAACCTTGATAATTGTCAT
TTAATTATATTTCAGGTGTCCTGAACAGGTCACTAGACTCTACATTGGGCAGCCTTTAAATATGAT
TCTTTGTAATGCTAAATAGCCTTTTTTTCTCTTTTTACTGCAACTTAATATTTCTATTTAGAACAC
AGAAAATGAAAATATTTAGAATAAGTTGTACATTTGATGACAAATAAATCACTATT
Homo sapiens SLC35A1 3′-UTR
SLC35A1-201 ENST00000369556
(SEQ ID NO: 305)
TTTTAGCCTCACGTGAGACTCCTTTTAAGACTAAACCATTTGCATTAAACTAGAGCCTTAAGTCAA
TCTCAGAAGGTAGCATAAACAAATAAAAATTAACTGTATGGCATGATCAGTGCGGTTATGTGGAAA
CAACAACAAACAAACGAAGCTATCTGAGTGAACTGCTAATACAGAAACTTAATGTAGACCTGTTTG
GGGTCTACTATTGTTTTAGAATGAAGGAATTGTATTATTGTGTGTATATATAATTTGTAAATAAAA
AGTATGGAGATGATACGGTGTTAAAAAAAATCATGGTAAGGCTACAATACTCAAGTAACAAGGTTT
GGGACAATGTCTAAGGGTTAAAGTGCCAAAGCCATTTCTGTACTAACTGTTCTCTTGTTCCGGTAC
CGGGGAGAAGGATGACCCCTCCTTATTCTCCAATTCATGTACAGTATTTTGTCCTAGCAGCATAAA
GACCTAGCTCTTTTCTTACAAGAGGCAGAAACAAGACAGGCTAGTTCATAAACAAACTGTGTAACT
TCTCAAAATGAATCTATTTCATAACTCGGACAATTTCTGGGTGGTGACTGAGTACCCCTTTAGTGA
GTACCCCTTTAGTGCTATATTTGTGCCATTCATTATCTGGTTCATATTTCTTTTCTGTTAGATGAT
ACACATTTCTTCAAAAAAATTTCTAATGTCACTTTTGTACTTTTTTAAATAAAGTATGTTTAACTG
TTGGGCTCTCAATAATTTGTGAAATTTCAGTGTTTTCTATAATGTTAATGGGGAAATTCAGCAATA
AACTTTATTTGT
Homo sapiens GLYATL2 3′-UTR
GLYATL2-003 ENST00000532258
(SEQ ID NO: 306)
TTGATTCCACTGTCCATTTCAAATCTTTCTTATCAGTAAAAAAACATTAATTCAAACACAAGCATT
GTGATCTACATTAGCACAAAATGCAACTGATTATCTAGGATCTGTGTATTACTTAAGCTCACCCTT
AACAGTTTTACCTTCCTTCTCCTCTGTATTCTTACAGAAAATTAGAAGCTCAATTTTATGGTCTCA
TAATTTCCTTTATGACAGACATCTCAGAATTAAAATCACCCAAAGCCAATCATTAGTGCCAAGATA
ACCCTTTAACGGCAACACTTTCTTAAATGAAGACTATTTCTTTCATGAAAAAATTCACTTTTATGA
CT
Homo sapiens STAT4 3′-UTR
STAT4-002 ENST00000392320
(SEQ ID NO: 307)
CAGGATAAACTCTGACGCACCAAGAAAGGAAGCAAATGAAAAAGTTTAAAGACTGTTCTTTGCCCA
ATAACCACATTTTATTTCTTCAGCTTTGTAAATACCAGGTTCTAGGAAATGTTTGACATCTGAAGC
TCTCTTCACACTCCCGTGGCACTCCTCAATTGGGAGTGTTGTGACTGAAATGCTTGAAACCAAAGC
TTCAGATAAACTTGCAAGATAAGACAACTTTAAGAAACCAGTGTTAATAACAATATTAACAG
Homo sapiens GULP1 3′-UTR
GULP1-002 ENST00000409609
(SEQ ID NO: 308)
CATCAAGAACAAGAAATCCTGATTCATGTTAAATGTGTTTGTATACACATGTCATTTATTATTATT
ACTTTAAGATAGGTATTATTCATGTGTCAATGTTTTTGAATATTTTAATATTTTGAAAATTTTCTC
AGTTAAATTTCCTCACCTTCACTATTGATCTGTAATTTTTATTTTAAAAACAGCTTACTGTAAAGT
AGATCATACTTTTATGTTCCTTTCTGTTTCTACTGTAGATGAATTTGTAATTGAAAGACATATTAT
ACAAAT
Homo sapiens GULP1 3′-UTR
GULP1-010 ENST00000409805
(SEQ ID NO: 309)
CATCAAGAACAAGAAATCCTGATTCATGTTAAATGTGTTTGTATACACATGTCATTTATTATTATT
ACTTTAAGATAGG
Homo sapiens EHHADH 3′-UTR
EHHADH-002 ENST00000456310
(SEQ ID NO: 310)
TTCAGTCTTCCAGATTATGCCTCACATGCTAGCATCAGGTAATGCTGACTGAATTTCAGTGAAATT
AAATCAAAAATCCAAAGTAAGATTGTTCTGAAATACAAAGCAAAATAAATAATCATTAGAATCTTC
TGTGTAACGACTCTAATGGTCAAATCTTTAGGAATGTGCTTCCTATGCCTCTGAATCTGTCCTTAT
CAGATAAATTCAATGCATGAACTTGTGTGAATATAATACCATAATAGCTAATGAAAGA
Homo sapiens NBEAL1 3′-UTR
NM_001114132.1
(SEQ ID NO: 311)
TTGTTATTTCCATTTTCTGTTATGATTACTGAAACCTGATTTATTGCTTTGTCACTTTAACCACAT
CTCTCAACTCTCTGCAATGTTGCAAGGCTTTTATCCCTGAAAATCATTTACAGATAACCACAATTT
GCTGTGGTATATAAACTAATTCTTGGTCTATACTAAGATGTATTTGAGAAAATACATTTGATTTGA
TTTTGTGGCCCATTCCTAAAGGTCATTGTATCCATTTTTAAAACAAACTAAAATGAGAACATTAGG
TTCAATTTTCTTATTATTCCAAATGATAAAATTTAAGATTTTTCTAATAAAAGAGTACAGATAATG
GGACAGTTGAGAGAGATGGCTTTAAATACATTCTTAAGTAATCATTTTCCTATTTACTGACCACTG
TAATGAAAATATATCAATTTATTTATGGAACTCCTGATTGGGGATAATATTTTAAAGGTATCTGTT
GCACACTTGGATTTTCAAAACTCGGTGAAAGTTACAAGTTTGCATGGTAAGAATAAAATAAGAATA
TTGAAACTGGTACATTAGCTAATTCTATTACTACTTAGCGTGTTTCTAATGAGAAGTTACTGAAAT
CTATTACTGTCCTTAATAAAAATTGAGTAGAAAAAAGTGGAACTAG
Homo sapiens KIAA1598 3′-UTR
NM_001258299.1
(SEQ ID NO: 312)
TCTGAATCAGAAAATACTGCAACTCCTTCCTCCTTTTGTCTGCCTTTTGTTCTCCAAAAGTAAGTG
GAAATTACATTTCCAAGAAAGGAAATGAAATAATTGCAGGCCCAAGGTCTGCAAAATATGTGTTGA
ATTGACAGTGAAAAGGATCCATGTGTTGACAGACACAGTTGTTAGATGCCATAAAGGCAGATGTGA
AGCTCAATTTATTTCTCATCTTGCTTG
Homo sapiens HFE 3′-UTR
HFE-006 ENST00000317896
(SEQ ID NO: 313)
CACGCAGCCTGCAGACTCACTGTGGGAAGGAGACAAAACTAGAGACTCAAAGAGGGAGTGCATTTA
TGAGCTCTTCATGTTTCAGGAGAGAGTTGAACCTAAACATAGAAATTGCCTGACGAACTCCTTGAT
TTTAGCCTTCTCTGTTCATTTCCTCAAAAAGATTTCCCCATTTAGGTTTCTGAGTTCCTGCATGCC
GGTGATCCCTAGCTGTGACCTCTCCCCTGGAACTGTCTCTCATGAACCTCAAGCTGCATCTAGAGG
CTTCCTTCATTTCCTCCGTCACCTCAGAGACATACACCTATGTCATTTCATTTCCTATTTTTGGAA
GAGGACTCCTTAAATTTGGGGGACTTACATGATTCATTTTAACATCTGAGAAAAGCTTTGAACCCT
GGGACGTGGCTAGTCATAACCTTACCAGATTTTTACACATGTATCTATGCATTTTCTGGACCCGTT
CAACTTTTCCTTTGAATCCTCTCTCTGTGTTACCCAGTAACTCATCTGTCACCAAGCCTTGGGGAT
TCTTCCATCTGATTGTGATGTGAGTTGCACAGCTATGAAGGCTGTACACTGCACGAATGGAAGAGG
CACCTGTCCCAGAAAAAGCATCATGGCTATCTGTGGGTAGTATGATGGGTGTTTTTAGCAGGTAGG
AGGCAAATATCTTGAAAGGGGTTGTGAAGAGGTGTTTTTTCTAATTGGCATGAAGGTGTCATACAG
ATTTGCAAAGTTTAATGGTGCCTTCATTTGGGATGCTACTCTAGTATTCCAGACCTGAAGAATCAC
AATAATTTTCTACCTGGTCTCTCCTTGTTCTGATAATGAAAATTATGATAAGGATGATAAAAGCAC
TTACTTCGTGTCCGACTCTTCTGAGCACCTACTTACATGCATTACTGCATGCACTTCTTACAATAA
TTCTATGAGATAGGTACTATTATCCCCATTTCTTTTTTAAATGAAGAAAGTGAAGTAGGCCGGGCA
C
Homo sapiens HFE 3′-UTR
HFE-004 ENST00000349999
(SEQ ID NO: 314)
CACGCAGCCTGCAGACTCACTGTGGGAAGGAGACAAAACTAGAGACTCAAAGAGGGAGTGCATTTA
TGAGCTCTTCATGTTTCAGGAGAGAGTTGAACCTAAACATAGAAATTGCCTGACGAACTCCTTGAT
TTTAGCCTTCTCTGTTCATTTCCTCAAAAAGATTTCCCCATTTAGGTTTCTGAGTTCCTGCATGCC
GGTGATCCCTAGCTGTGACCTCTCCCCTGGAACTGTCTCTCATGAACCTCAAGCTGCATCTAGAGG
CTTCCTTCATTTCCTCCGTCACCTCAGAGACATACACCTATGTCATTTCATTTCCTATTTTTGGAA
GAGGACTCCTTAAATTTGGGGGACTTACATGATTCATTTTAACATCTGAGAAAAGCTTTGAACCCT
GGGACGTGGCTAGTCATAACCTTACCAGATTTTTACACATGTATCTATGCATTTTCTGGACCCGTT
CAACTTTTCCTTTGAATCCTCTCTCTGTGTTACCCAGTAACTCATCTGTCACCAAGCCTTGGGGAT
TCTTCCATCTGATTGTGATGTGAGTTGCACAGCTATGAAGGCTGTACACTGCACGAATGGAAGAGG
CACCTGTCCCAGAAAAAGCATCATGGCTATCTGTGGGTAGTATGATGGGTGTTTTTAGCAGGTAGG
AGGCAAATATCTTGAAAGGGGTTGTGAAGAGGTGTTTTTTCTAATTGGCATGAAGGTGTCATACAG
ATTTGCAAAGTTTAATGGTGCCTTCATTTGGGATG
Homo sapiens HFE 3′-UTR
HFE-005 ENST00000397022
(SEQ ID NO: 315)
CACGCAGCCTGCAGACTCACTGTGGGAAGGAGACAAAACTAGAGACTCAAAGAGGGAGTGCATTTA
TGAGCTCTTCATGTTTCAGGAGAGAGTTGAACCTAAACATAGAAATTGCCTGACGAACTCCTTGAT
TTTAGCCTTC
Homo sapiens HFE 3′-UTR
HFE-012 ENST00000336625
(SEQ ID NO: 316)
CACGCAGCCTGCAGACTCACTGTGGGAAGGA
Homo sapiens KIAA1324L 3′-UTR
KIAA1324L-005 ENST00000416314
(SEQ ID NO: 317)
AGAGACAGTGCTGTAGCCTTGAGACTAATGAACAAAGAAACCTGCTCTAGTTTTACAGGACCATAT
TTTAGGGTCTGTCCTCATACCTGTCACATTGGTGATCTCACAGAGGAGGGCCATGCCGCTGAAAAG
GGAAGGAGATTGAAACATTTGATTGCCTTATCACATGGTCAAGTACCTTGCCAAATAAAGGAAAGC
AAATGATTTGGGTCTCAACTGAAGATGAAGCTCAACTCAGGAAGAGATTTATCTGTATATACACAT
AACTGAAAACCAAGTTTAAGCCCACCAATGCACTGCTGATGCATGCCATATAATTAATGGGTAACT
TTTATTCTTTATGATGTCTACATAACAAGTGTGATTTGGAAGGCACATGTGAGCATATGCATTA
Homo sapiens MANSC1 NM_018050 3′-UTR
(SEQ ID NO: 318)
GGATGGAACTCGGTGTCTCTTAATTCATTTAGTAACCAGAAGCCCAAATGCAATGAGTTTCTGCTG
ACTTGCTAGTCTTAGCAGGAGGTTGTATTTTGAAGACAGGAAAATGCCCCCTTCTGCTTTCCTTTT
TTTTTTTTGGAGACAGAGTCTTGCTTTGTTGCCCAGGCTGGAGTGCAGTAGCACGATCTCGGCTCT
CACCGCAACCTCCGTCTCCTGGGTTCAAGCGATTCTCCTGCCTCAGCCTCCTAAGTATCTGGGATT
ACAGGCATGTGCCACCACACCTGGGTGATTTTTGTATTTTTAGTAGAGACGGGGTTTCACCATGTT
GGTCAGGCTGGTCTCAAACTCCTGACCTAGTGATCCACCCTCCTCGGCCTCCCAAAGTGCTGGGAT
TACAGGCATGAGCCACCACAGCTGGCCCCCTTCTGTTTTATGTTTGGTTTTTGAGAAGGAATGAAG
TGGGAACCAAATTAGGTAATTTTGGGTAATCTGTCTCTAAAATATTAGCTAAAAACAAAGCTCTAT
GTAAAGTAATAAAGTATAATTGCCATATAAATTTCAAAATTCAACTGGCTTTTATGCAAAGAAACA
GGTTAGGACATCTAGGTTCCAATTCATTCACATTCTTGGTTCCAGATAAAATCAACTGTTTATATC
AATTTCTAATGGATTTGCTTTTCTTTTTATATGGATTCCTTTAAAACTTATTCCAGATGTAGTTCC
TTCCAATTAAATATTTG

Preferably, the at least one 5′-UTR element comprises or consists of a nucleic acid sequence which has an identity of at least about 1, 2, 3, 4, 5, 10, 15, 20, 30 or 40%, preferably of at least about 50%, preferably of at least about 60%, preferably of at least about 70%, more preferably of at least about 80%, more preferably of at least about 90%, even more preferably of at least about 95%, even more preferably of at least about 99% to a nucleic acid sequence selected from the group consisting of SEQ ID NO: 25 or SEQ ID NO: 30 and SEQ ID NOs: 319 to 382 or the corresponding DNA or RNA sequence, respectively, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to a nucleic acid sequence selected from the group consisting of SEQ ID NO: 25 or SEQ ID NO: 30 and SEQ ID NOs: 319 to 382 or the corresponding DNA or RNA sequence, respectively:

Homo sapiens LTA4H 5′-UTR
LTA4H-001 ENST00000228740
(SEQ ID NO: 319)
AAGAAACTTCCTTTCCCGGCGTGCACCGCGAATCCCTCCTCCTCTTCTTTACCTCTCTCCCTCCTC
CTCAGGTTCTCTATCGACGAGTCTGGTAGCTGAGCGTTGGGCTGTAGGTCGCTGTGCTGTGTGATC
CCCCAGAGCC
Homo sapiens DECR1 5′-UTR
DECR1-001 ENST00000220764
(SEQ ID NO: 320)
TCCAGCCCCGAGAACTTTGTTCTTTTTGTCCCGCCCCCTGCGCCCAACCGCCTGCGCCGCCTTCCG
GCCCGAGTTCTGGAGACTCAAC
Homo sapiens PIGK 5′-UTR
(SEQ ID NO: 321)
ACTGCCTCCGCCCCTTCAGGTGCGGGAAGTCTGAAGCCGGTAAAC
Homo sapiens BRP44L 5′-UTR
BRP44L-001
(SEQ ID NO: 322)
GTCGTGAGGCGGGCCTTCGGGCTGGCTCGCCGTCGGCTGCCGGGGGGTTGGCCGGGGTGTCATTGG
CTCTGGGAAGCGGCAGCAGAGGCAGGGACCACTCGGGGTCTGGTGTCGGCACAGCC
Homo sapiens ACADSB 5′-UTR
ACADSB-004 NM_001609.3 ENST00000368869
(SEQ ID NO: 323)
AGGGATTAAGGGGGGGTGTGTGCGGGGCGGGTACTGAGTGGGCGGGGCCTTGCTCGGGTAACTCCC
AGGGGCTGGCTAGAGACCCAGAGGCGCAGAGCGGAGAGGCCTGCGGCGAGG
Homo sapiens SUPT3H 5′-UTR
SUPT3H-006 ENST00000371459
(SEQ ID NO: 324)
CACAGCCGAGTCACCTTTTCCCTTTCTACACTCCACACTCTCAGTCCCCCACCCCGCCCCTTTCCA
AGCGTGTCCCGGGCCGCAGCAGCAGAAACCGCACCATCTCCACCCCCACATTCTCCTCGCGGGAAG
CGCAGCAGTGCCTCCAAGGGTTCTTAAAGCAGAG
Homo sapiens TMEM14A 5′-UTR
NM_014051.3
(SEQ ID NO: 325)
GTTTCCAGGAGGGAGCGGCCTTTGCTCAGCGCGAGACGGCTGGGCGCCGAGTGGGACAGCGCTGGT
GCGGAGACTGCTTCCGGACTCCAGGTACCGCGCTTGGCGGCAGCTGGCCCCAGACTTCTGTCTTTT
CAGCTGCAGTGAAGGCTCGGGGCTGCAGAATTGCAACCTTGCCA
Homo sapiens C9orf46 5′-UTR
AF225420.1
(SEQ ID NO: 326)
GAGCGAGGCCCGGTCCCTGCAGCGGGCGAAAGGAGCCCGGGCCTGGAGGTTTGCGTACCGGTCGCC
TGGTCCCGGCACCAGCGCCGCCCAGTGTGGTTTCCCATAAGGAAGCTCTTCTTCCTGCTTGGCTTC
CACCTTTAACCCTTCCACCTGGGAGCGTCCTCTAACACATTCAGACTACAAGTCCAGACCCAGGAG
AGCAAGGCCCAGAAAGAGGTCAAA
Homo sapiens ANXA4 5′-UTR
NM_001153.3
(SEQ ID NO: 327)
GCCCCAGGTGCGCTTCCCCTAGAGAGGGATTTTCCGGTCTCGTGGGCAGAGGAACAACCAGGAACT
TGGGCTCAGTCTCCACCCCACAGTGGGGCGGATCCGTCCCGGATAAGACCCGCTGTCTGGCCCTGA
GTAGGGTGTGACCTCCGCAGCCGCAGAGGAGGAGCGCAGCCCGGCCTCGAAGAACTTCTGCTTGGG
TGGCTGAACTCTGATCTTGACCTAGAGTC
Homo sapiens IFI6 5′-UTR
NM_022873.2
(SEQ ID NO: 328)
CCAGCCTTCAGCCGGAGAACCGTTTACTCGCTGCTGTGCCCATCTATCAGCAGGCTCCGGGCTGAA
GATTGCTTCTCTTCTCTCCTCCAAGGTCTAGTGACGGAGCCCGCGCGCGGCGCCACC
Homo sapiens C2orf34 5′-UTR
CAMKMT-008 ENST00000402247
(SEQ ID NO: 329)
TCCTGGCAGGGGACGAGCTGCGGCGGTGGCACCTCCGGGTGTGGAAGGCTCCAGTGAG
Homo sapiens C2orf34 5′-UTR
NM_024766.3
(SEQ ID NO: 330)
GAGGGTGCCGGGCGTCACAGGTCCTGACAGGGAAGAAGTTGGCAGGTCCTGGCAGGGGACGAGCTG
CGGCGGTGGCACCTCCGGGTGTGGAAGGCTCCAGTGAG
Homo sapiens ALDH6A1 5′-UTR
ALDH6A1-002 ENST00000350259
(SEQ ID NO: 331)
AGTGCTTCTGGGCAGTAGAGGCGCGGGGTGCGGAGCTAGGGCGGCCGAGAGCC
Homo sapiens CCDC53 5′-UTR
CCDC53-002 ENST00000545679
(SEQ ID NO: 332)
GGAAGGGCCCCGGAGGCGGGCACTTGGGGGGAAAGTTGAGACGTGATTACCGGGTTGGGCGGGCCC
CATCTGGGAGGGGTTTGTGGGTGAACTCGGGGTCCACCGCCCGCTGAGGAG
Homo sapiens CASP1 5′-UTR
NM_001257119.1
(SEQ ID NO: 333)
ATACTTTCAGTTTCAGTCACACAAGAAGGGAGGAGAGAAAAGCC
Homo sapiens NDUFB6 5′-UTR
NM_182739.2
(SEQ ID NO: 334)
GTAATAACCGCGCGCGGCGCTCGGCGTTCCCGCAAGGTCGCTTTGCAGAGCGGGAGCGCGCTTAAG
TAACTAGTCCGTAGTTCGAGGGTGCGCCGTGTCCTTTTGCGTTGGTACCAGCGGCGAC
Homo sapiens BCKDHB 5′-UTR
BCKDHB-002 ENST00000369760
(SEQ ID NO: 335)
AGGCGGCGTGCGGCTGCATAGCCTGAGAATCCCGGTGGTGAGCGGGG
Homo sapiens BCKDHB 5′-UTR
NM_001164783.1
(SEQ ID NO: 336)
CTACGTGAGTGCCGGACCGCTGAGTGGTTGTTAGCCAAG
Homo sapiens BBS2 5′-UTR
NM_031885.3
(SEQ ID NO: 337)
CACAGAAGGCGCCGAGGCTCCACCGCGCAGCCGCAAAAAGAGCGGACGGGTCTGCGCCGCCGCAGG
AGGAGCAGGCGGTACCTGGACGGGTTCGTCCCGGGCTGTTTCGCGTCCGGCCTGAGGCGGCTGGGG
CCGCGCAGGTAGTGTCCCTGCACTTCTTGCCCGGGCGCGTGAGGCCAGCTCCGCTGCGCTTGTCTC
CAGCTTCCAGCCCTCCTCCCCTAAGCCGCCGCCATC
Homo sapiens HERC5 5′-UTR
HERC5-001 ENST00000264350
(SEQ ID NO: 338)
TCAGTAGCTGAGGCTGCGGTTCCCCGACGCCACGCAGCTGCGCGCAGCTGGTTCCCGCTCTGCAGC
GCAACGCCTGAGGCAGTGGGCGCGCTCAGTCCCGGGACCAGGCGTTCTCTCCTCTCGCCTCTGGGC
CTGGGACCCCGCAAAGCGGCG
Homo sapiens FAM175A 5′-UTR
NM_139076.2
(SEQ ID NO: 339)
ACCACAGGGTCTTGCCTCCGCGCGCCCCGCCCTCGTCCTCTTGTGTAGCCTGAGGCGGCGGTAGC
Homo sapiens NT5DC1 5′-UTR
NT5DC1-002 ENST00000319550
(SEQ ID NO: 340)
CGGTCCTGTCCCGCAGCGTCCCGCCAGCCAGCTCCTTGCACCCTTCGCGGCCGAGGCGCTCCCTGG
TGCTCCCCGCGCAGCC
Homo sapiens RAB7A 5′-UTR
RAB7A-001 ENST00000265062
(SEQ ID NO: 341)
GTCTCGTGACAGGTACTTCCGCTCGGGGCGGCGGCGGTGGCGGAAGTGGGAGCGGGCCTGGAGTCT
TGGCCATAAAGCCTGAGGCGGCGGCAGCGGCGGAGTTGGCGGCTTGGAGAGCTCGGGAGAGTTCCC
TGGAACCAGAACTTGGACCTTCTCGCTTCTGTCCTCCGTTTAGTCTCCTCCTCGGCGGGAGCCCTC
GCGACGCGCCCGGCCCGGAGCCCCCAGCGCAGCGGCCGCGTTTGAAGG
Homo sapiens AGA 5′-UTR
AGA-001 ENST00000264595
(SEQ ID NO: 342)
AGGGACGCCTGAGCGAACCCCCGAGAGAGCGGGCGTGGGCGCCAGGCGGGCGGGGCACTGGGGATT
AATTGTTCGGCGATCGCTGGCTGCCGGGACTTTTCTCGCGCTGGTCTCTTCGGTGGTCAGGG
Homo sapiens TPK1 5′-UTR
TPK1-001 ENST00000360057
(SEQ ID NO: 343)
AAGGCTCCTCAGCCGAGCGCCGAGCGGTCGATCGCCGTAGCTCCCGCAGCCTGCGATCTCCAGTCT
GTGGCTCCTACCAGCCATTGTAGGCCAATAATCCGTT
Homo sapiens MBNL3 5′-UTR
MBNL3-001 ENST00000370839
(SEQ ID NO: 344)
AATTCATTTTTAATCCTTTAATAGTCCACAGTAATATTGTCCTAAAGAGGGTACATTGGATTTTAA
TTTTGCTTTCAAT
Homo sapiens MCCC2 5′-UTR
MCCC2-001 ENST00000340941
(SEQ ID NO: 345)
AGAATCAGAGAAACCTTCTCTGGGGCTGCAAGGACCTGAGCTCAGCTTCCGCCCCAGCCAGGGAAG
CGGCAGGGGAAAGCACCGGCTCCAGGCCAGCGTGGGCCGCTCTCTCGCTCGGTGCCCGCCGCC
Homo sapiens CAT 5′-UTR
CAT-001 ENST00000241052
(SEQ ID NO: 346)
ACTCGGGGCAACAGGCAGATTTGCCTGCTGAGGGTGGAGACCCACGAGCCGAGGCCTCCTGCAGTG
TTCTGCACAGCAAACCGCACGCT
Homo sapiens ANAPC4 5′-UTR
ANAPC4-001 ENST00000315368
(SEQ ID NO: 347)
CCCGACGCCGGAAGTGCCTGGAGCGCGCGACAGCGGCGGGGCGGGGCGGCCTGGAGGCTGTGGCGC
GCGGCCGGCAGAGGGAGGGGAGAGGCCACTGGGGCCGTGTTAGTCTGCCGGTGGGGACTCTTGCAG
GGCCGTCCCC
Homo sapiens PHKB 5′-UTR
PHKB-002 ENST00000323584
(SEQ ID NO: 348)
GGCCAAGGCGGCGACCGGAGCGCG
Homo sapiens ABCB7 5′-UTR
ABCB7-001 ENST00000253577
(SEQ ID NO: 349)
CTCGGTTCCTCTTTCCTCGCTCAAG
Homo sapiens GPD2 5′-UTR
GPD2-002 ENST00000438166
(SEQ ID NO: 350)
CCCGCGCGCCTCGCTGGGAGCACCCGGGCCGAGGCTCTGATTCTGGGGGGAGGCCGACTCCACCCT
GGCTGGAGGAACTGGGTGCTCCTGCCCGCTGGCCCCTCGCGCGTGAGGATCTATCTCAGGCTAAGA
A
Homo sapiens TMEM38B 5′-UTR
TMEM38B-001 ENST00000374692
(SEQ ID NO: 351)
GCTGGAGCCGGCGCGGAGGAGCGGGCGGCCGCGGCTGTGCCCTCTCCTACTCCTCACCGCGCGAGC
GCGGGGAACCAGTAGCCGCGGCTGCTTCGGTTGCCGCGGTCGGTGGTCGTT
Homo sapiens NFU1 5′-UTR
NM_001002755.2
(SEQ ID NO: 352)
GGGAAAGGTTCCCCGGCCTCTCTTGGTCAGGGTGACGCAGTAGCCTGCAAACCTCGGCGCGTAGGC
CACCGCACTTATCCGCAGCAGGACCGCCCGCAGCCGGTAGGGTGGGCTCTTCCCAGTGCCCGCCCA
GCTACCGGCCAGCCTGCGGCTGCGCAGATCTTTCGTGGTTCTGTCAGGGAGACCCTTAGGCACTCC
GGACTAAG
Homo sapiens LOC128322/NUTF2 5′-UTR
NM_005796.1
(SEQ ID NO: 353)
GGAAGGGACAGTCGGCCGCAGACCGCGCTGGGTTGCCGCTGCCGCTGCCGCCATCGTGCCAGCCCC
TCGGGTCTCCGTGAGGCCGGGTGACGCTCCAGA
Homo sapiens NUBPL 5′-UTR
NM_025152.2
(SEQ ID NO: 354)
ACTCCGCGCCACCCGCGACAGTTTCCCAGCAGGGCTCACAGCAGCGTTCCGCGTC
Homo sapiens LANCL1 5′-UTR
LANCL1-004 ENST00000233714
(SEQ ID NO: 355)
GAGAAGGGCTTCAGGACGCGGGAGGCGCACTTGCTTCAAGTCGCGGGCGTGGGAACGGGGCTTGCT
TCCGGCGTC
Homo sapiens PIR 5′-UTR
PIR-002 ENST00000380420
(SEQ ID NO: 356)
CCTCCCGCCTCCTCTAGGCCGCCGGCCGCGAAGCGCTGAGTCACGGTGAGGCTACTGGACCCACAC
TCTCTTAACCTGCCCTCCCTGCACTCGCTCCCGGCGGCTCTTCGCGTCACCCCCGCCGCTAAGGCT
CCAGGTGCCGCTACCGCAGCCCCTCCATCCTCTACAGCTCAGCATCAGAACACTCTCTTTTTAGAC
TCCGAT
Homo sapiens CTBS 5′-UTR
NM_004388.2
(SEQ ID NO: 357)
GACGCGCAGCAGGCCCCGCCCACCCAGGCGGTAGGAACCCACTCCGGCCCGCTAGACCTGCTGCT
Homo sapiens GSTM4 5′-UTR
NM_000850.4
(SEQ ID NO: 358)
AAGCTGGCGAGGCCGAGCCCCTCCTAGTGCTTCCGGACCTTGCTCCCTGAACACTCGGAGGTGGCG
GTGGATCTTACTCCTTCCAGCCAGTGAGGATCCAGCAACCTGCTCCGTGCCTCCCGCGCCTGTTGG
TTGGAAGTGACGACCTTGAAGATCGGCCGGTTGGAAGTGACGACCTTGAAGATCGGCGGGCGCAGC
GGGGCCGAGGGGGCGGGTCTGGCGCTAGGTCCAGCCCCTGCGTGCCGGGAACCCCAGAGGAGGTCG
CAGTTCAGCCCAGCTGAGGCCTGTCTGCAGAATCGACACCAACCAGCATC
Mus musculus Ndufa1 5′-UTR
Ndufa1-001 ENSMUST00000016571
(SEQ ID NO: 359)
GCCGGAAGAGAGGTAAAGCCGGGTCACCTCTGAGGAGCCGGTGACGGGTTGGCGTGCGAGTAACGG
TGCGGAG
Mus musculus Atp5e 5′-UTR
NM_025983
(SEQ ID NO: 360)
CCCACCCCTTCCGCTACTCAGGCCTGACCTTCCTGCTGCCGGGCCGGTTTGAGGCTACTCTGAAGC
GACCCAGCGGTTCTGCCCGACGCGCCCGCTCGAGACACC
Mus musculus Gstm5 5′-UTR
NM_010360
(SEQ ID NO: 361)
GAGACAGTTCGGTCGCGTCAGCCCGGCCCACAGCGTCCAGTATAAAGTTAGCCGCCCACAGTCCAT
CGCTGTATCCCCGAAGGGGCTAAGATCGCCCAAA
Mus musculus Cbr2 5′-UTR
NM_007621
(SEQ ID NO: 362)
ATAAAAGCTGAGCCCATCTCTTGCTTCGGAAGAAGCTGGTGTCAGCAGC
Mus musculus Anapc13 5′-UTR
NM_181394
(SEQ ID NO: 363)
GTGACCCAGAAGAAGGGCGGGGCCGGGAGGAAGCCGACGCGCGCGCAGTGGGCCTGACAAGATCAA
AGCTGCAGGAGG
Mus musculus Ndufa7 5′-UTR
NM_023202
(SEQ ID NO: 364)
TCGGAGCGGAAGGAAT
Mus musculus Atp5k 5′-UTR
NM_007507
(SEQ ID NO: 365)
CGAAGGTCACGGACAAA
Mus musculus Cox4i1 5′-UTR
NM_009941
(SEQ ID NO: 366)
CTTCCGGTCGCGAGCACCCCAGGGTGTAGAGGGCGGTCGCGGCGGTCGCCTGGGCAGCGGTGGCAG
A
Mus musculus Ndufs6 5′-UTR
NM_010888
(SEQ ID NO: 367)
TTGGTACGACGCGTGGGGTCAAGGGTCACCGGCAAG
Mus musculus Sec61b 5′-UTR
NM_024171
(SEQ ID NO: 368)
AGAGCCTGTATCTACGAGAGTTCTGAGTGCTCGGCAACTTCACGACTTCCCTCTTCCTGCCTCCTG
TGCCCACCGTTCTTAGGCATCAGC
Mus musculus Snrpd2 5′-UTR
NM_026943
(SEQ ID NO: 369)
AAGGCTGGAGCAACGCGCTTGGAGGCGGGAGTGATCTGCGAGCGAAACCTACACC
Mus musculus Mgst3 5′-UTR
NM_025569
(SEQ ID NO: 370)
ACTGCTGTGCTTCTCAGGTCTGTACCAGGCGCACGAAGGTGAGCCAGAGCCAAG
Mus musculus Mp68 (2010107E04Rik) 5′-UTR
NM_027360
(SEQ ID NO: 371)
CTTTCCCATTCTGTAGCAGAATTTGGTGTTGCCTGTGGTCTTGGTCCCGCGGAG
Mus musculus Prdx4-001, 5′-UTR
NM_016764
(SEQ ID NO: 372)
GCGCGGTCTCCAGCGCGCCGTTTTAGCTGGCTGCCTGGCGGCAGGGGACTCTGTGCTTTAGCAGAG
GGACGTGTTTTCGCGCTTGCTTGGTC
Mus musculus Pgcp 5′-UTR
NM_176073
(SEQ ID NO: 373)
GCTGTCCTGGCACACAAAGAAGCCAGGCCTGCAGACTACTGGGGCTCCGGGCTGTTCCTGAGGCCT
CTGGAGGCCCGCCCTGTGGCTCCAGTGCGCTCTGAGGACCTTCCTGGTCCCGCCCCCGAACGTGCC
TGTGGTCTGCAGGCCTCACCGGGTGTTGTGGCCGCTGCTGCTCCGCAGAGCCTCGTGATCAGGAAG
AAAAGCAACTAGGAACA
Mus musculus Myeov2 5′-UTR
NM_001163425
(SEQ ID NO: 374)
AGAAGGGGCTGGCCGGAAGTGAGCGCAACGCCGCCTTGTCGAG
Mus musculus Ndufa4 5′-UTR
NM_010886
(SEQ ID NO: 375)
GTCCGCTCAGCCAGGTTGCAGAAGCGGCTTAGCGTGTGTCCTAATCTTCTCTCTGCGTGTAGGTAG
GCCTGTGCCGCAAAC
Mus musculus Ndufs5 5′-UTR
NM_001030274
(SEQ ID NO: 376)
ACGGCAGGCGTCTGCGTCCTCCCGCAGCCGGCGGTCGGGAATTGCACCAGGGACCTGACAAGGGCA
CTGCAGAGCC
Mus musculus Gstm1 5′-UTR
NM_010358
(SEQ ID NO: 377)
CTGCCTTCCGCTTTAGGGTCTGCTGCTCTGGTTACAGACCTAGGAAGGGGAGTGCCTAATTGGGAT
TGGTGCAGGGTTGGGAGGGACCCGCTGTTTTGTCCTGCCCACGTTTCTCTAGTAGTCTGTATAAAG
TCACAACTCCAAACACACAGGTCAGTCCTGCTGAAGCCAGTTTGAGAAGACCACAGCACCAGCACC
Mus musculus Atp5o 5′-UTR
NM_138597
(SEQ ID NO: 378)
CTGGCGCGCGCGCGTGCGCTCTGGCGCCAGTAGTCTCTTTTCATTTGGGTTTGACCTACAGCCGCC
CGGGAAAAG
Mus musculus Tspo 5′-UTR
NM_009775
(SEQ ID NO: 379)
GTCAGCGGCTACCAACCTCTGTGCGCAGTGTCCTTCACGGAACAACCAGCGACTGCGTGAGCGGGG
CTGTGGATCTTTCCAGAACATCAGTTGCAATCACC
Mus musculus Taldo1 5′-UTR
NM_011528
(SEQ ID NO: 380)
GACGCGCGGGGCATTGTGGGTTAGCACGCACCGGCTACCGCCTCAGCTGTTCGCGTTTCGCC
Mus musculus Bloc1s1 5′-UTR
NM_015740
(SEQ ID NO: 381)
GTGACGCCTTCCGGGTGAGCCAAGGCATAGTCCAGTTCCTGCAGCCTTAGGGAGGGGTCCGCCGTG
CCCACACCCAGCCAGACTCGACC
Mus musculus Hexa 5′-UTR
NM_010421
(SEQ ID NO: 382)
AGCTGACCGGGGCTCACGTGGGCTCAGCCTGCTGGAAGGGGAGCTGGCCGGTGGGCC

Preferably, the at least one 3′-UTR element of the artificial nucleic acid molecule according to the present invention comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the 3′-UTR sequence of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), Ybx1 (Y-Box binding protein 1), Ndufb8 (NADH dehydrogenase (ubiquinone) 1 beta subcomplex 8), and CNTN1 (contactin 1; whereby CNTN1-004 is particularly preferred). Most preferably, the at least one 3′-UTR element of the artificial nucleic acid molecule according to the present invention comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to a sequence selected from the group consisting of SEQ ID NO: 1 to SEQ ID NO: 24, or the corresponding RNA sequences, respectively:

(Mus musculus GNAS 3′-UTR)
SEQ ID NO: 1
GAAGGGAACACCCAAATTTAATTCAGCCTTAAGCACAATTAATTAAGAGTGAAACGTAATGTACAA
GCAGTTGGTCACCCACCATAGGGCATGATCAACACCGCAACCTTTCCTTTTTCCCCCAGTGATTCT
GAAAAACCCCTCTTCCCTTCAGCTTGCTTAGATGTTCCAAATTTAGTAAGCTTAAGGCGGCCTACA
GAAGAAAAAGAAAAAAAAGGCCACAAAAGTTCCCTCTCACTTTCAGTAAATAAAATAAAAGCAGCA
ACAGAAATAAAGAAATAAATGAAATTCAAAATGAAATAAATATTGTTTGTGCAGCATTAAAAAATC
AATAAAAATTAAAAATGAGCA
(Mus musculus GNAS 3′-UTR)
SEQ ID NO: 2
GAAGGGAACACCCAAATTTAATTCAGCCTTAAGCACAATTAATTAAGAGTGAAACGTAATTGTACA
AGCAGTTGGTCACCCACCATAGGGCATGATCAACACCGCAACCTTTCCTTTTTCCCCCAGTGATTC
TGAAAAACCCCTCTTCCCTTCAGCTTGCTTAGATGTTCCAAATTTAGTAAGCTTAAGGCGGCCTAC
AGAAGAAAAAGAAAAAAAAGGCCACAAAAGTTCCCTCTCACTTTCAGTAAATAAAATAAAAGCAGC
AACAGAAATAAAGAAATAAATGAAATTCAAAATGAAATAAATATTGTGTTGTGCAGCATTAAAAAA
TCAATAAAAATTAAAAATGAGCA
(Homo sapiens GNAS 3′-UTR)
SEQ ID NO: 3
GAAGGGAACCCCCAAATTTAATTAAAGCCTTAAGCACAATTAATTAAAAGTGAAACGTAATTGTAC
AAGCAGTTAATCACCCACCATAGGGCATGATTAACAAAGCAACCTTTCCCTTCCCCCGAGTGATTT
TGCGAAACCCCCTTTTCCCTTCAGCTTGCTTAGATGTTCCAAATTTAGAAAGCTTAAGGCGGCCTA
CAGAAAAAGGAAAAAAGGCCACAAAAGTTCCCTCTCACTTTCAGTAAAAATAAATAAAACAGCAGC
AGCAAACAAATAAAATGAAATAAAAGAAACAAATGAAATAAATATTGTGTTGTGCAGCATTAAAAA
AAATCAAAATAAAAATTAAATGTGAGCAAAGAATGAAAAAAAAAAAAAAAAAAAA
(Homo sapiens GNAS 3′-UTR)
SEQ ID NO: 4
TGGAGGACGCCGTCCAGATTCTCCTTGTTTTCATGGATTCAGGTGCTGGAGAATCTGGTAAAAGCA
CCATTGTGAAGCAGATGAGGATCCTGCATGTTAATGGGTTTAATGGAGAGGGCGGCGAAGAGGACC
CGCAGGCTGCAAGGAGCAACAGCGATGGCAGTGAGAAGGCAACCAAAGTGCAGGACATCAAAAACA
ACCTGAAAGAGGCGATTGAAACCATTGTGGCCGCCATGAGCAACCTGGTGCCCCCCGTGGAGCTGG
CCAACCCCGAGAACCAGTTCAGAGTGGACTACATCCTGAGTGTGATGAACGTGCCTGACTTTGACT
TCCCTCCCGAATTCTATGAGCATGCCAAGGCTCTGTGGGAGGATGAAGGAGTGCGTGCCTGCTACG
AACGCTCCAACGAGTACCAGCTGATTGACTGTGCCCAGTACTTCCTGGACAAGATCGACGTGATCA
AGCAGGCTGACTATGTGCCGAGCGATCAGGACCTGCTTCGCTGCCGTGTCCTGACTTCTGGAATCT
TTGAGACCAAGTTCCAGGTGGACAAAGTCAACTTCCACATGTTTGACGTGGGTGGCCAGCGCGATG
AACGCCGCAAGTGGATCCAGTGCTTCAACGATGTGACTGCCATCATCTTCGTGGTGGCCAGCAGCA
GCTACAACATGGTCATCCGGGAGGACAACCAGACCAACCGCCTGCAGGAGGCTCTGAACCTCTTCA
AGAGCATCTGGAACAACAGATGGCTGCGCACCATCTCTGTGATCCTGTTCCTCAACAAGCAAGATC
TGCTCGCTGAGAAAGTCCTTGCTGGGAAATCGAAGATTGAGGACTACTTTCCAGAATTTGCTCGCT
ACACTACTCCTGAGGATGCTACTCCCGAGCCCGGAGAGGACCCACGCGTGACCCGGGCCAAGTACT
TCATTCGAGATGAGTTTCTGAGGATCAGCACTGCCAGTGGAGATGGGCGTCACTACTGCTACCCTC
ATTTCACCTGCGCTGTGGACACTGAGAACATCCGCCGTGTGTTCAACGACTGCCGTGACATCATTC
AGCGCATGCACCTTCGTCAGTACGAGCTGCTCTAAGAAGGGAACCCCCAAATTTAATTAAAGCCTT
AAGCACAATTAATTAAAAGTGAAACGTAATTGTACAAGCAGTTAATCACCCACCATAGGGCATGAT
TAACAAAGCAACCTTTCCCTTCCCCCGAGTGATTTTGCGAAACCCCCTTTTCCCTTCAGCTTGCTT
AGATGTTCCAAATTTAGAAAGCTTAAGGCGGCCTACAGAAAAAGGAAAAAAGGCCACAAAAGTTCC
CTCTCACTTTCAGTAAAAATAAATAAAACAGCAGCAGCAAACAAATAAAATGAAATAAAAGAAACA
AATGAAATAAATATTGTGTTGTGCAGCATTAAAAAAAATCAAAATAAAAATTAAATGTGAGCAAAG
AATGAAAAAAAAAAAAAAAAAAAA
(Mus musculus MORN2 3′-UTR)
SEQ ID NO: 5
ACCTGCTGCCTTAACGCTGAGATGTGGCCTCTGCAACCCCCCTTAGGCAAAGCAACTGAACCTTCT
GCTAAAGTGACCTGCCCTCTTCCGTAAGTCCAATAAAGTTGTCATGCACCC
(Mus musculus MORN2 3′-UTR)
SEQ ID NO: 6
ACCTGCTGCCTTAACGCTGAGATGTGGCCTCTGCAACCCCCCTTAGGCAAAGCAACTGAACCTTCT
GCTAAAGTGACCTGCCCTCTTCCGTAAGTCCAATAAAGTTGTCATGCACCCACAAAAAAAAAAAAA
AAA
(Homo sapiens MORN2 3′-UTR)
SEQ ID NO: 7
CATGTAGATGTGATGTTAAATTAAAGTTGAAATGTAGTAATTGAAGCTTTTAGTTGTAAGGAAAGC
AACTTAATCTGTTATTTGAAATGACTTCATACACTACCCCTATAAGTTTGCCAATAAAACCATCAC
CTGCTTACACCTTTTTGAACTTTATATTCATTGTCTTACAATTAGTTTAAAATAAATGACATGATT
CAAAAAAAAAAA
(Mus musculus GSTM1 3′-UTR)
SEQ ID NO: 8
GCCCTTGCTACACGGGCACTCACTAGGAGGACCTGTCCACACTGGGGATCCTGCAGGCCCTGGGTG
GGGACAGCACCCTGGCCTTCTGCACTGTGGCTCCTGGTTCTCTCTCCTTCCCGCTCCCTTCTGCAG
CTTGGTCAGCCCCATCTCCTCACCCTCTTCCCAGTCAAGTCCACACAGCCTTCATTCTCCCCAGTT
TCTTTCACATGGCCCCTTCTTCATTGGCTCCCTGACCCAACCTCACAGCCCGTTTCTGCGAACTGA
GGTCTGTCCTGAACTCACGCTTCCTAGAATTACCCCGATGGTCAACACTATCTTAGTGCTAGCCCT
CCCTAGAGTTACCCCGAAGGTCAATACTTGAGTGCCAGCCTGTTCCTGGTGGAGTAGCCTCCCCAG
GTCTGTCTCGTCTACAATAAAGTCTGAAACACACTTGCCATG
(Mus musculus GSTM1 3′-UTR)
SEQ ID NO: 9
GCCCTTGCTACACGGGCACTCACTAGGAGGACCTGTCCACACTGGGGATCCTGCAGGCCCTGGGTG
GGGACAGCACCCTGGCCTTCTGCACTGTGGCTCCTGGTTCTCTCTCCTTCCCGCTCCCTTCTGCAG
CTTGGTCAGCCCCATCTCCTCACCCTCTTCCCAGTCAAGTCCACACAGCCTTCATTCTCCCCAGTT
TCTTTCACATGGCCCCTTCTTCATTGGCTCCCTGACCCAACCTCACAGCCCGTTTCTGCGAACTGA
GGTCTGTCCTGAACTCACGCTTCCTAGAATTACCCCGATGGTCAACACTATCTTAGTGCTAGCCCT
CCCTAGAGTTACCCCGAAGGTCAATACTTGAGTGCCAGCCTGTTCCTGGTGGAGTAGCCTCCCCAG
GTCTGTCTCGTCTACAATAAAGTCTGAAACACACTTGCCATGAAAAAAAAAAAAAAAAA
(Homo sapiens GSTM1 3′-UTR)
SEQ ID NO: 10
GGCCTTGAAGGCCAGGAGGTGGGAGTGAGGAGCCCATACTCAGCCTGCTGCCCAGGCTGTGCAGCG
CAGCTGGACTCTGCATCCCAGCACCTGCCTCCTCGTTCCTTTCTCCTGTTTATTCCCATCTTTACT
CCCAAGACTTCATTGTCCCTCTTCACTCCCCCTAAACCCCTGTCCCATGCAGGCCCTTTGAAGCCT
CAGCTACCCACTATCCTTCGTGAACATCCCCTCCCATCATTACCCTTCCCTGCACTAAAGCCAGCC
TGACCTTCCTTCCTGTTAGTGGTTGTGTCTGCTTTAAAGGGCCTGCCTGGCCCCTCGCCTGTGGAG
CTCAGCCCCGAGCTGTCCCCGTGTTGCATGAAGGAGCAGCATTGACTGGTTTACAGGCCCTGCTCC
TGCAGCATGGTCCCTGCCTTAGGCCTACCTGATGGAAGTAAAGCCTCAACCACAAAAAAAAAAAAA
AAAAAAAAAAAAAAAAAAAAAAAAAAAAAAAAAAAAAAAAAAAAAAAAAAAAAAAAAAAAAAAAAA
AAA
(Mus musculus NDUFA1 3′-UTR)
SEQ ID NO: 11
GGAAGCATTTTCCTGGCTGATTAAAAGAAATTACTCAGCTATGGTCATCTGTTCCTGTTAGAAGGC
TATGCAGCATATTATATACTATGCGCATGTTATGAAATGCATAATAAAAAATTTTAAAAAATCTAA
A
(Homo sapiens NDUFA1 3′-UTR)
SEQ ID NO: 12
GGAAGCATTTTCCTGATTGATGAAAAAAATAACTCAGTTATGGCCATCTACCCCTGCTAGAAGGTT
ACAGTGTATTATGTAGCATGCAATGTGTTATGTAGTGCTTAATAAAAATAAAATGAAAAAAATGCA
AAAAAAAAAAAAAAAA
(Mus musculus CBR2 3′-UTR)
SEQ ID NO: 13
TCTGCTCAGTTGCCGCGGACATCTGAGTGGCCTTCTTAGCCCCACCCTCAGCCAAAGCATTTACTG
ATCTCGTGACTCCGCCCTCATGCTACAGCCACGCCCACCACGCAGCTCACAGTTCCACCCCCATGT
TACTGTCGATCCCACAACCACTCCAGGCGCAGACCTTGTTCTCTTTGTCCACTTTGTTGGGCTCAT
TTGCCTAAATAAACGGGCCACCGCGTTACCTTTAACTAT
(Mus musculus YBX1 3′-UTR)
SEQ ID NO: 14
ATGCCGGCTTACCATCTCTACCATCATCCGGTTTGGTCATCCAACAAGAAGAAATGAATATGAAAT
TCCAGCAATAAGAAATGAACAAAGATTGGAGCTGAAGACCTTAAGTGCTTGCTTTTTGCCCGCTGA
CCAGATAACATTAGAACTATCTGCATTATCTATGCAGCATGGGGTTTTTATTATTTTTACCTAAAG
ATGTCTCTTTTTGGTAATGACAAACGTGTTTTTTAAGAAAAAAAAAAAAGGCCTGGTTTTTCTCAA
TACACCTTTAACGGTTTTTAAATTGTTTCATATCTGGTCAAGTTGAGATTTTTAAGAACTTCATTT
TTAATTTGTAATAAAGTTTACAACTTGATTTTTTCAAAAAAGTCAACAAACTGCAAGCACCTGTTA
ATAAAGGTCTTAAATAATAA
(Mus musculus YBX1 3′-UTR)
SEQ ID NO: 15
ATGCCGGCTTACCATCTCTACCATCATCCGGTTTGGTCATCCAACAAGAAGAAATGAATATGAAAT
TCCAGCAATAAGAAATGAACAAAGATTGGAGCTGAAGACCTTAAGTGCTTGCTTTTTGCCCTCTGA
CCAGATAACATTAGAACTATCTGCATTATCTATGCAGCATGGGGTTTTTATTATTTTTACCTAAAG
ATGTCTCTTTTTGGTAATGACAAACGTGTTTTTTAAGAAAAAAAAAAAAAAGGCCTGGTTTTTCTC
AATACACCTTTAACGGTTTTTAAATTGTTTCATATCTGGTCAAGTTGAGATTTTTAAGAACTTCAT
TTTTAATTTGTAATAAAGTTTACAACTTGATTTTTTCAAAAAAGTCAACAAACTGCAAGCACCTGT
TAATAAAGGTCTTAAATAATAA
(Homo sapiens YBX1 3′-UTR)
SEQ ID NO: 16
ATGCCGGCTTACCATCTCTACCATCATCCGGTTTAGTCATCCAACAAGAAGAAATATGAAATTCCA
GCAATAAGAAATGAACAAAAGATTGGAGCTGAAGACCTAAAGTGOTTGOTTTTTGOCCOTTGACCA
GATAAATAGAACTATCTGCATTATCTATGCAGCATGGGGTTTTTATTATTTTTACCTAAAGACGTC
TCTTTTTGGTAATAACAAACGTGTTTTTTAAAAAAGCCTGGTTTTTCTCAATACGCCTTTAAAGGT
TTTTAAATTGTTTCATATCTGGTCAAGTTGAGATTTTTAAGAACTTCATTTTTAATTTGTAATAAA
AGTTTACAACTTGATTTTTTCAAAAAAGTCAACAAACTGCAAGCACCTGTTAATAAAGGTCTTAAA
TAATAAAAAAAAAAAAAAA
(Mus musculus Ndufb8 3′-UTR)
SEQ ID NO: 17
GGAGGCTTGATGGGCTTTTTGCCCTCGTTCCTAGAGGCTTAACCATAATAAAATCCCTAATAAAGC
(Homo sapiens Ndufb8 3′-UTR)
SEQ ID NO: 18
GGAGGCTTCGTGGGCTTTTGGGTCCTCTAACTAGGACTCCCTCATTCCTAGAAATTTAACCTTAAT
GAAATCCCTAATAAAACTCAGTGCTGTGTTATTTGTGCCTCAAAAAAAAAAAAAAAAAA
(Homo sapiens Ndufb8 3′-UTR)
SEQ ID NO: 19
GTGAGGAAGAGGAGTGCTGTTCCTGCCTTCCTAGCCCAGCTGGGTCTGACCAGAGGCTACTGTGTA
CCCATTTACCATGCGTGATTGTTAACTCAGAGTGGGGTGTAGCCAGGTATTGACTGAATGTATGTT
CTTGCTGACCTGTGTTTTTTTCTGTAGGGACCAAAGCAGTATCCTTACAATAATCTGTACCTGGAA
CGAGGCGGTGATCCCTCCAAAGAACCAGAGCGGGTGGTTCACTATGAGATCTGAGGAGGCTTCGTG
GGCTTTTGGGTCCTCTAACTAGGACTCCCTCATTCCTAGAAATTTAACCTTAATGAAATCCCTAAT
AAAACTCAGTGCTGTGTTATTTGTGCCTCAAAAAAAAAAAAAAAAAA
(Homo sapiens CNTN1-004 3′-UTR)
SEQ ID NO: 20
TCGTTGACACTCACCATTTCTGTGAAAGACTTTTTTTTTTTTTAACATATTATACTAGATTTGACT
AACTCAATCTTGTAGCTTCTGCAGTTCTCCCCACCCCCAACCTAGTTCTTAGAGTATGTTTCCCCT
TTTGAAACATGTAAACATACTTTGGGCATAAATATTTTTTAAAATATAACTATAATGCTTCACTAA
TACCTTAAAAATGCCTAGTGAACTAACTCAGTACATTATATAATGGCCAAGTGAAAGTTTTGTGTT
TTCATGTCCTGTTTTTCTTTGAAATTATATAGCCCAGAAATTAGCTCATTATCTGAAAAACGTATA
AGAACTGATGAATTGTATAATACAGGAGTATTGCCATTGAATGTACTGTTTGATTTATTCAAGCAG
GTAATGAACAATGTTGTCAAACTCTCTAATGAGACATCATAATTAGGACATAAGCTAAAAGGGGCA
TTACTCCGGCAGTCTTTTTTTCTTAATCCTAGTACCATACATATTCTTTGGCATGAAAGAATGAAA
AGCATTAGTAAACAACTGAAGTCCTACCATGGCTCTGTAGGGTTTTTGGAACAATTCCTGGAATTG
GAAAGTGAAAATGGATAGCATGTGGGGGAAACCCTCATCTGAGTAGCAAGATTTTAGTAAAGATGA
CTAAGCCATTAACAGCATGCATTCATATTTAATTTTATTGACTCCTGCCATCAGCTTTTGTAGATC
TTTTGGGTGGAAGGTTGTGATTTTTACTGGGAGGACTTGAGTAGAAGTGGATGATTAAAATTGAGG
AGTATATAATTCTTTCTGGGACTGCTTAAATGTTATTGTTTGAAAATGCCTTCACTTTCCCCCTTT
GGTCAAAGAGATGTGCTTAAAATTCTTATTCCTTCACAATAAATAATTTTGATTTTCTTAGACA
(Homo sapiens CNTN1-004 3′-UTR)
SEQ ID NO: 21
TCGTTGACACTCACCATTTCTGTGAAAGACTTTTTTTTTTTTAACATATTATACTAGATTTGACTA
ACTCAATCTTGTAGCTTCTGCAGTTCTCCCCACCCCCAACCTAGTTCTTAGAGTATGTTTCCCCTT
TTGAAACATGTAAACATACTTTGGGCATAAATATTTTTTAAAATATAACTATAATGCTTCACTAAT
ACCTTAAAAATGCCTAGTGAACTAACTCAGTACATTATATAATGGCCAAGTGAAAGTTTTGTGTTT
TCATGTCCTGTTTTTCTTTGAAATTATATAGCCCAGAAATTAGCTCATTATCTGAAAAACGTATGA
AGAACTGATGAATTGTATAATACAGGAGTATTGCCATTGAATGTACTGTTTGATTTATTCAAGCAG
GTAATGAACAATGTTGTCAAACTCTCTAATGAGACATCATAATTAGGACATAAGCTAAAAGGGGCA
TTACTCCGGCAGTCTTTTTTTCTTAATCCTAGTACCATACATATTCTTTGGCATGAAAGAATGAAA
AGCATTAGTAAACAACTGAAGTCCTACCATGGCTCTGTAGGGTTTTTGGAACAATTCCTGGAATTG
GAAAGTGAAAATGGATAGCATGTGGGGGAAACCCTCATCTGAGTAGCAAGATTTTAGTAAAGATGA
CTAAGCCATTAACAGCATGCATTCATATTTAATTTTATTGACTCCTGCCATCAGCTTTTGTAGATC
GTTTGGGTGGAAGGTTGTGATTTTTACTGGGAGGACTTGAGTAGAAGTGGATGATTAAAATTGAGG
AGTATATAATTCTTTCTGGGACTGCTTAAATGTTATTGTTTGAAAATACCTTCACTTTCCCCCTTT
GGTCAAAGAGATGTGCTTAAAATTCTTATTCCTTCACAATAAATAATTTTGATTTTCTTAGACA
(Homo sapiens CNTN1-004 3′-UTR)
SEQ ID NO: 22
TTTTTTCGTTGACACTCACCATTTCTGTGAAAGACTTTTTTTTTTTTTAACATATTATACTAGATT
TGACTAACTCAATCTTGTAGCTTCTGCAGTTCTCCCCACCCCCAACCTAGTTCTTAGAGTATGTTT
CCCCTTTTGAAACATGTAAACATACTTTGGGCATAAATATTTTTTAAAATATAACTATAATGCTTC
ACTAATACCTTAAAAATGCCTAGTGAACTAACTCAGTACATTATATAATGGCCAAGTGAAAGTTTT
GTGTTTTCATGTCCTGTTTTTCTTTGAAATTATATAGCCCAGAAATTAGCTCATTATCTGAAAAAC
GTATGAAGAACTGATGAATTGTATAATACAGGAGTATTGCCATTGAATGTACTGTTTGATTTATTC
AAGCAGGTAATGAACAATGTTGTCAAACTCTCTAATGAGACATCATAATTAGGACATAAGCTAAAA
GGGGCATTACTCCGGCAGTCTTTTTTTCTTAATCCTAGTACCATACATATTCTTTGGCATGAAAGA
ATGAAAAGCATTAGTAAACAACTGAAGTCCTACCATGGCTCTGTAGGGTTTTTGGAACAATTCCTG
GAATTGGAAAGTGAAAATGGATAGCATGTGGGGGAAACCCTCATCTGAGTAGCAAGATTTTAGTAA
AGATGACTAAGCCATTAACAGCATGCATTCATATTTAATTTTATTGACTCCTGCCATCAGCTTTTG
TAGATCTTTTGGGTGGAAGGTTGTGATTTTTACTGGGAGGACTTGAGTAGAAGTGGATGATTAAAA
TTGAGGAGTATATAATTCTTTCTGGGACTGCTTAAATGTTATTGTTTGAAAATGCCTTCACTTTCC
CCCTTTGGTCAAAGAGATGTGCTTAAAATTCTTATTCCTTCACAATAAATAATTTTGATTTTCTTA
GACA
(Homo sapiens CNTN1-004 3′-UTR)
SEQ ID NO: 23
ATGTGTTGTGACAGCTGCTGTTCCCATCCCAGCTCAGAAGACACCCTTCAACCCTGGGATGACCAC
AATTCCTTCCAATTTCTGCGGCTCCATCCTAAGCCAAATAAATTATACTTTAACAAACTATTCAAC
TGATTTACAACACACATGATGACTGAGGCATTCGGGAACCCCTTCATCCAAAAGAATAAACTTTTA
AATGGATATAAATGATTTTTAACTCGTTCCAATATGCCTTATAAACCACTTAACCTGATTCTGTGA
CAGTTGCATGATTTAACCCAATGGGACAAGTTACAGTGTTCAATTCAATACTATAGGCTGTAGAGT
GAAAGTCAAATCACCATATACAGGTGCTTTAAATTTAATAACAAGTTGTGAAATATAATAGAGATT
GAAATGTTGGTTGTATGTGGTAAATGTAAGAGTAATACAGTCTCTTGTACTTTCCTCACTGTTTTG
GGTACTGCATATTATTGAATGGCCCCTATCATTCATGACATCTTGAGTTTTCTTGAAAAGACAATA
GAGTGTAACAAATATTTTGTCAGAAATCCCATTATCAAATCATGAGTTGAAAGATTTTGACTATTG
AAAACCAAATTCTAGAACTTACTATCAGTATTCTTATTTTCAAAGGAAATAATTTTCTAAATATTT
GATTTTCAGAATCAGTTTTTTAATAGTAAAGTTAACATACCATATAGATTTTTTTTTACTTTTATA
TTCTACTCTGAAGTTATTTTATGCTTTTCTTATCAATTTCAAATCTCAAAAATCACAGCTCTTATC
TAGAGTATCATAATATTGCTATATTTGTTCATATGTGGAGTGACAAATTTTGAAAAGTAGAGTGCT
TCCTTTTTTATTGAGATGTGACAGTCTTTACATGGTTAGGAATAAGTGACAGTTAAGTGAATATCA
CAATTACTAGTATGTTGGTTTTTCTGCTTCATTCCTAAGTATTACGTTTCTTTATTGCAGATGTCA
GATCAAAAAGTCACCTGTAGGTTGAAAAAGCTACCGTATTCCATTTTGTAAAAATAACAATAATAA
TAATAATAATAATTAGTTTTAAGCTCATTTCCCACTTCAATGCAATACTGAAAACTGGCTAAAAAT
ACCAAATCAATATACTGCTAATGGTACTTTGAAGAGTATGCAAAACTGGAAGGCCAGGAGGAGGCA
AATAATATGTCTTTCCGATGGTGTCTCCCAAGTGTTGGTGCTTTGGGTTTTTATAAGTTGTGAAAA
GGAAGATGCACATTTCTTCATTCTCCATGGTGTGCATGGAAATGTGTTTGAGTGTGGATGTAAAAG
AAATCGAGTAATAAAGAATTAGCTGGCTTGTGAAATAGTGCAGTGTTGGATGCTTCAAGAGGTATA
ATCCTATTTTATTAGCACAAACTTGCTAGCTAATTAGAGTTTATCTTTTTAGAAAGGACACCGTAT
AGGTTCGTAAAAAATATTTACAGGAAGCAAAATAGATCTATTACTACTTTACCGACTTTACCCCCT
TTCTTTAATTTGTATAATTTTTGTACTATATATCGATGTGTAAATGTTTAGAGTCTTCATTATGAA
AATATCAATAAATATTTCATTAGTTTACATTTAACTCTGGTATAAAATGAAACTTTTAAAAATAAG
TGAAATGGATGATTTCCCAGTGGAAGTATGTCAACAGTCTTAAGATCATTGCCAGATTTCATAAAA
TATTTAAGTATTTGAAAAAGAAACAAAATGTCTTCATACTTTAGGGAAACGAATACCCTGTATACC
TTCTGTACAAATGTTTGTGTTTTCATTGTTACACTTTGGGGTTTTACTTTTGCAATGTGACCCATG
TTGGGCATTTTTATATAATCAACAACTAAATCTTTTGCCAAATGCATGCTTGCCTTTTATTTTCTA
ATATATGATAATAACGAGCAAAACTGGTTAGATTTTGCATGAAATGGTTCTGAAAGGTAAGAGGAA
AACAGACTTTGGAGGTTGTTTAGTTTTGAATTTCTGACAGAGATAAAGTAGTTTAAAATCTCTCGT
ACACTGATAACTCAAGCTTTTCATTTTCTCATACAGTTGTACAGATTTAACTGGGACCATCAGTTT
TAAACTGTTGTCAAGCTAACTAATAATCATCTGCTTTAAGACGCAAGATTCTGAATTAAACTTTAT
ATAGGTATAGATACATCTGTTGTTTCTTTGTATTTCAGGAAAGGTGATAGTAGTTTTATTTGATAC
TGATAAATATTGAATTGATTTTTTAGTTATTTTTTATCATTTTTTCAATGGAGTAGTATAGGACTG
TGCTTTGTCCTTTTTATGAATGAAAAAATTAGTATAAAGTAATAAATGTCTTATGTTACCCAAGAA
AAAA
(Homo sapiens CNTN1-004 3′-UTR)
SEQ ID NO: 24
TCGTTGACACTCACCATTTCTGTGAAAGACTTTTTTTTTTTTAACATATTATACTAGATTTGACTA
ACTCAATCTTGTAGCTTCTGCAGTTCTCCCCACCCCCAACCTAGTTCTTAGAGTATGTTTCCCCTT
TTGAAACATGTAAACATACTTTGGGCATAAATATTTTTTAAAATATAACTATAATGCTTCACTAAT
ACCTTAAAAATGCCTAGTGAACTAACTCAGTACATTATATAATGGCCAAGTGAAAGTTTTGTGTTT
TCATGTCCTGTTTTTCTTTGAAATTATATAGCCCAGAAATTAGCTCATTATCTGAAAAACGTATGA
AGAACTGATGAATTGTATAATACAGGAGTATTGCCATTGAATGTACTGTTTGATTTATTCAAGCAG
GTAATGAACAATGTTGTCAAACTCTCTAATGAGACATCATAATTAGGACATAAGCTAAAAGGGGCA
TTACTCCGGCAGTCTTTTTTTCTTAATCCTAGTACCATACATATTCTTTGGCATGAAAGAATGAAA
AGCATTAGTAAACAACTGAAGTCCTACCATGGCTCTGTAGGGTTTTTGGAACAATTCCTGGAATTG
GAAAGTGAAAATGGATAGCATGTGGGGGAAACCCTCATCTGAGTAGCAAGATTTTAGTAAAGATGA
CTAAGCCATTAACAGCATGCATTCATATTTAATTTTATTGACTCCTGCCATCAGCTTTTGTAGATC
GTTTGGGTGGAAGGTTGTGATTTTTACTGGGAGGACTTGAGTAGAAGTGGATGATTAAAATTGAGG
AGTATATAATTCTTTCTGGGACTGCTTAAATGTTATTGTTTGAAAATACCTTCACTTTCCCCCTTT
GGTCAAAGAGATGTGCTTAAAATTCTTATTCCTTCACAATAAATAATTTTGATTTTCTTAGACAGG
TTTGTGTTTAGGTATGAGTTTCTCTTTTACTTCATCTAGCAATTCTCTCTGTGGTCAGAAGAACTC
TGAAGAAAGCTTTGAGGGAAATGAATATAACTCTTAAATTATTATATGTGTGTGTATATATATAGT
TTAACTTTAAAAATAATTTATTAGTCATCATAAAGAAATAAATGTCTCTGGCTCAAGATGTTACTT
ATTTCCTTCTTTTATATTTTCTAGTCTCAATTACTGTTCCAAAAGGAGCTATCTTAGAACTTAGAC
TAGAGATCCAGATTAA

Preferably, the at least one 5′-UTR element of the artificial nucleic acid molecule according to the present invention comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the 5′-UTR sequence of a transcript of MP68 (RIKEN cDNA 2010107E04 gene), or NDUFA4 (NADH dehydrogenase (ubiquinone) 1 alpha subcomplex 4). Most preferably, the at least one 5′-UTR element of the artificial nucleic acid molecule according to the present invention comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to a sequence according to SEQ ID NO: 25 or SEQ ID NO: 30, or the corresponding RNA sequence, respectively:

(Mus musculus MP68 5′-UTR)
SEQ ID NO: 25
CTTTCCCATTCTGTAGCAGAATTTGGTGTTGCCTGTGGTCTTGGTCCCG
CGGAG
(Homo sapiens MP68 5′-UTR)
SEQ ID NO: 26
CTTCCCGGCATCCCCTGCGCGCGCCTGCGCGCTCGGTGACCTTTCCGAG
TTGGCTGCAGATTTGTGGTGCGTTCTGAGCCGTCTGTCCTGCGCCAAG
(Homo sapiens MP68 5′-UTR)
SEQ ID NO: 27
CTTCCCGGCATCCCCTGCGCGCGCCTGCGCGCTCGGTGACCTTTCCGAG
TTGGCTGCAGATTTGTGGTGCGTTCTGAGCCGTCTGTCCTGCGCCAAGG
GAGCGTACCTTGGCCTTGAGAGGTTCAGCTGCCTAACCCAGAGGCTACG
CAGAGTTAGAGAAGCCAGAGTCCAAGCCAAGAACTCTGACTCCACATCC
AGTCCCTTCTCTCCTTTATAACTCAAGTTTCCTTGCGCCACACTGCCCT
CCACGTTATGCTGTACATGACAACTTGGGTGAGGCAACAGGGAAGCTGA
AAAGAGATCATACGGTGCTGA
(Mus musculus NDUFA4 5′-UTR)
SEQ ID NO: 28
GTCCGCTCAGCCAGGTTGCAGAAGCGGCTTAGCGTGTGTCCTAATCTTC
TCTCTGCGTGTAGGTAGGCCTGTGCCGCAAAC
(Homo sapiens NDUFA4 5′-UTR)
SEQ ID NO: 29
GUCCGCUCAGCCAGGUUGCAGAAGCGGCUUAGCGUGUGUCCUAAUCUUC
UCUCUGCGUGUAGGUAGGCCUGUGCCGCAAAC
(Homo sapiens NDUFA4 5′-UTR)
SEQ ID NO: 30
GGGTCCTTCAGGTAGGAGGTCCTGGGTGACTTTGGAAGTCCGTAGTGTC
TCATTGCAGATAATTTTTAGCTTAGGGCCTGGTGGCTAGGTCGGTTCTC
TCCTTTCCAGTCGGAGACCTCTGCCGCAAAC

The at least one 3′-UTR element of the artificial nucleic acid molecule according to the present invention may also comprise or consist of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the nucleic acid sequence of the 3′-UTR of a transcript of a gene, such as to the 3′-UTR of a sequence according to SEQ ID NOs: 1 to 24 and SEQ ID NOs: 49 to 318, wherein the fragment is preferably a functional fragment or a functional variant fragment as described above. Such fragment preferably exhibits a length of at least about 3 nucleotides, preferably of at least about 5 nucleotides, more preferably of at least about 10, 15, 20, 25 or 30 nucleotides, even more preferably of at least about 50 nucleotides, most preferably of at least about 70 nucleotides. In a preferred embodiment, the fragment or variant thereof exhibits a length of between 3 and about 500 nucleotides, preferably of between 5 and about 150 nucleotides, more preferably of between 10 and 100 nucleotides, even more preferably of between 15 and 90, most preferably of between 20 and 70. Preferably, said variants, fragments or variant fragments are functional variants, functional fragments, or functional variant fragments of the 3′-UTR, prolong protein production from the artificial nucleic acid molecule according to the invention with an efficiency of at least 30%, preferably with an efficiency of at least 40%, more preferably of at least 50%, more preferably of at least 60%, even more preferably of at least 70%, even more preferably of at least 80%, most preferably of at least 90% of the protein production prolonging efficiency exhibited by an artificial nucleic acid molecule comprising the nucleic acid sequence selected from the group consisting of SEQ ID NO: 1 to SEQ ID NO: 24.

The at least one 5′-UTR element of the artificial nucleic acid molecule according to the present invention may also comprise or consist of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the nucleic acid sequence of the 5′-UTR of a transcript of a gene, such as to the 5′-UTR of a sequence according to SEQ ID NO: 25 or SEQ ID NO: 30 and SEQ ID NOs: 319 to 382, wherein the fragment is preferably a functional fragment or a functional variant fragment as described above. Such fragment preferably exhibits a length of at least about 3 nucleotides, preferably of at least about 5 nucleotides, more preferably of at least about 10, 15, 20, 25 or 30 nucleotides, even more preferably of at least about 50 nucleotides, most preferably of at least about 70 nucleotides. In a preferred embodiment, the fragment or variant thereof exhibits a length of between 3 and about 500 nucleotides, preferably of between 5 and about 150 nucleotides, more preferably of between 10 and 100 nucleotides, even more preferably of between 15 and 90, most preferably of between 20 and 70. Preferably, said variants, fragments or variant fragments are functional variants, functional fragments, or functional variant fragments of the 5′-UTR, increase protein production from the artificial nucleic acid molecule according to the invention with an efficiency of at least 30%, preferably with an efficiency of at least 40%, more preferably of at least 50%, more preferably of at least 60%, even more preferably of at least 70%, even more preferably of at least 80%, most preferably of at least 90% of the protein production increasing efficiency exhibited by an artificial nucleic acid molecule comprising the nucleic acid sequence selected from the group consisting of SEQ ID NO: 25 to SEQ ID NO: 30.

Preferably, the at least one 3′-UTR element and/or the at least one 5′-UTR element of the artificial nucleic acid molecule according to the present invention exhibits a length of at least about 3 nucleotides, preferably of at least about 5 nucleotides, more preferably of at least about 10, 15, 20, 25 or 30 nucleotides, even more preferably of at least about 50 nucleotides, most preferably of at least about 70 nucleotides. The upper limit for the length of the at least one 3′-UTR element and/or the at least one 5′-UTR element may be 500 nucleotides or less, e.g. 400, 300, 200, 150 or 100 nucleotides. For other embodiments the upper limit may be chosen within the range of 50 to 100 nucleotides. For example, the fragment or variant thereof may exhibit a length of between 3 and about 500 nucleotides, preferably of between 5 and about 150 nucleotides, more preferably of between 10 and 100 nucleotides, even more preferably of between 15 and 90, most preferably of between 20 and 70.

Furthermore, the artificial nucleic acid molecule according to the present invention may comprise more than one 3′-UTR elements and/or more than one 5′-UTR elements as described above. For example, the artificial nucleic acid molecule according to the present invention may comprise one, two, three, four or more 3′-UTR elements, and/or one, two, three, four or more 5′-UTR elements, wherein the individual 3′-UTR elements may be the same or they may be different, and similarly, the individual 5′-UTR elements may be the same or they may be different. For example, the artificial nucleic acid molecule according to the present invention may comprise two essentially identical 3′-UTR elements as described above, e.g. two 3′-UTR elements comprising or consisting of a nucleic acid sequence, which is derived from the 3′-UTR of a transcript of a gene, such as from a sequence according to SEQ ID NOs: 1 to 24 and SEQ ID NO: 49 to 318, or from a fragment or variant of the 3′-UTR of a transcript of a gene, functional variants thereof, functional fragments thereof, or functional variant fragments thereof as described above. Accordingly, for example, the artificial nucleic acid molecule according to the present invention may comprise two essentially identical 5′-UTR elements as described above, e.g. two 5′-UTR elements comprising or consisting of a nucleic acid sequence, which is derived from the 5′-UTR of a transcript of a gene, such as from a sequence according to SEQ ID NOs: 25 to 30 and SEQ ID NO: 319 to 382, or from a fragment or variant of the 5′-UTR of a transcript of a gene, functional variants thereof, functional fragments thereof, or functional variant fragments thereof as described above.

Surprisingly, the inventors found that an artificial nucleic acid molecule comprising a 3′-UTR element as described above and/or a 5′-UTR element as described above may represent or may provide an mRNA molecule, which allows for increased, prolonged and/or stabilized protein production. Thus, a 3′-UTR element as described herein and/or a 5′-UTR element as described herein may improve stability of protein expression from an mRNA molecule and/or improve translational efficiency.

In particular, the artificial nucleic acid molecule according to the invention may comprise (i) at least one 3′-UTR element and at least one 5′-UTR element, which prolongs and/or increases protein production; (ii) at least one 3′-UTR element, which prolongs and/or increases protein production, but no 5′-UTR element, which prolongs and/or increases protein production; or (iii) at least one 5′-UTR element, which prolongs and/or increases protein production, but no 3′-UTR element, which prolongs and/or increases protein production.

However, in particular in case (ii) and (iii), but possibly also in case (i), the artificial nucleic acid molecule according to the present invention may further comprise one or more “further 3′-UTR elements and/or 5′-UTR elements”, i.e. 3′-UTR elements and/or 5′-UTR elements which do not fulfil the requirements as described above. For example, an artificial nucleic acid molecule according to the invention, which comprises a 3′-UTR element according to the present invention, i.e. a 3′-UTR element which prolongs and/or increases protein production from said artificial nucleic acid molecule, may additionally comprise any further 3′-UTR and/or any further 5′-UTR, in particular a further 5′-UTR, e.g. a 5′-TOP UTR, or any other 5′-UTR or 5′-UTR element. Similarly for example, an artificial nucleic acid molecule according to the invention, which comprises a 5′-UTR element according to the present invention, i.e. a 5′-UTR element which prolongs and/or increases protein production from said artificial nucleic acid molecule, may additionally comprise any further 3′-UTR and/or any further 5′-UTR, in particular a further 3′-UTR, e.g. a 3′-UTR derived from a 3′-UTR of an albumin gene, particularly preferably a 3′-UTR comprising a sequence according to SEQ ID NO. 31 or 32, in particular to SEQ ID NO. 32, or any other 3′-UTR or 3′-UTR element.

If additionally to the inventive at least one 5′-UTR element and/or to the inventive at least one 3′-UTR element, which prolongs and/or increases protein production, a further 3′-UTR (element) and/or a further 5′-UTR (element) are present in the artificial nucleic acid molecule according to the invention, the further 5′-UTR (element) and/or the further 3′-UTR (element) may interact with the inventive 3′-UTR element and/or inventive 5′-UTR element and, thus, support the increasing and/or prolonging effect of the inventive 3′-UTR element and/or of the inventive 5′-UTR element, respectively. Such further 3′-UTR and/or 5′-UTR (elements) may further support stability and translational efficiency. Moreover, if both, an inventive 3′-UTR element and an inventive 5′-UTR element are present in the artificial nucleic acid molecule according to the invention, the prolonging and/or increasing effect of the inventive 5′-UTR element and the inventive 3′-UTR element result preferably in enhanced and prolonged protein production in a synergistic way.

Preferably, the further 3′-UTR comprises or consists of a nucleic acid sequence which is derived from a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene, or from a variant of a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene according to SEQ ID No. 1369-1390 of the patent application WO2013/143700 whose disclosure is incorporated herein by reference. In a particularly preferred embodiment, the further 3′-UTR comprises or consists of a nucleic acid sequence which is derived from a 3′-UTR of an albumin gene, preferably a vertebrate albumin gene, more preferably a mammalian albumin gene, most preferably a human albumin gene according to SEQ ID NO. 31:

(Human albumin 3′-UTR; corresponding to
SEQ ID No: 1369 of the patent application
WO2013/143700)
SEQ ID NO. 31:
CATCACATTT AAAAGCATCT CAGCCTACCA TGAGAATAAG
AGAAAGAAAA TGAAGATCAA AAGCTTATTC ATCTGTTTTT
CTTTTTCGTT GGTGTAAAGC CAACACCCTG TCTAAAAAAC
ATAAATTTCT TTAATCATTT TGCCTCTTTT CTCTGTGCTT
CAATTAATAA AAAATGGAAA GAATCT

In this context it is particularly preferred that the inventive nucleic acid molecule comprises a further 3′-UTR element derived from the nucleic acids according to SEQ ID No. 1369-1390 of the patent application WO2013/143700 or a fragment, homolog or variant thereof.

Most preferably the further 3′-UTR comprises the nucleic acid sequence derived from a fragment of the human albumin gene according to SEQ ID NO. 32:

(albumin7 3′-UTR; corresponding to
SEQ ID No: 1376 of the patent application
WO2013/143700)
SEQ ID NO. 32:
CATCACATTTAAAAGCATCTCAGCCTACCATGAGAATAAGAGAAAGAAA
ATGAAGATCAATAGCTTATTCATCTCTTTTTCTTTTTCGTTGGTGTAAA
GCCAACACCCTGTCTAAAAAACATAAATTTCTTTAATCATTTTGCCTCT
TTTCTCTGTGCTTCAATTAATAAAAAATGGAAAGAACCT

In this context it is particularly preferred that the further 3′-UTR of the inventive artificial nucleic acid molecule comprises or consists of the nucleic acid sequence according to SEQ ID NO. 32, or a corresponding RNA sequence.

The further 3′-UTR may also comprise or consist of a nucleic acid sequence derived from a ribosomal protein coding gene, whereby ribosomal protein coding genes from which a further 3′-UTR may be derived include, but are not limited to, ribosomal protein L9 (RPL9), ribosomal protein L3 (RPL3), ribosomal protein L4 (RPL4), ribosomal protein L5 (RPL5), ribosomal protein L6 (RPL6), ribosomal protein L7 (RPL7), ribosomal protein L7a (RPL7A), ribosomal protein L11 (RPL11), ribosomal protein L12 (RPL12), ribosomal protein L13 (RPL13), ribosomal protein L23 (RPL23), ribosomal protein L18 (RPL18), ribosomal protein L18a (RPL18A), ribosomal protein L19 (RPL19), ribosomal protein L21 (RPL21), ribosomal protein L22 (RPL22), ribosomal protein L23a (RPL23A), ribosomal protein L17 (RPL17), ribosomal protein L24 (RPL24), ribosomal protein L26 (RPL26), ribosomal protein L27 (RPL27), ribosomal protein L30 (RPL30), ribosomal protein L27a (RPL27A), ribosomal protein L28 (RPL28), ribosomal protein L29 (RPL29), ribosomal protein L31 (RPL31), ribosomal protein L32 (RPL32), ribosomal protein L35a (RPL35A), ribosomal protein L37 (RPL37), ribosomal protein L37a (RPL37A), ribosomal protein L38 (RPL38), ribosomal protein L39 (RPL39), ribosomal protein, large, P0 (RPLP0), ribosomal protein, large, P1 (RPLP1), ribosomal protein, large, P2 (RPLP2), ribosomal protein S3 (RPS3), ribosomal protein S3A (RPS3A), ribosomal protein S4, X-linked (RPS4X), ribosomal protein S4, Y-linked 1 (RPS4Y1), ribosomal protein S5 (RPS5), ribosomal protein S6 (RPS6), ribosomal protein S7 (RPS7), ribosomal protein S8 (RPS8), ribosomal protein S9 (RPS9), ribosomal protein S10 (RPS10), ribosomal protein Si1 (RPS11), ribosomal protein S12 (RPS12), ribosomal protein S13 (RPS13), ribosomal protein S15 (RPS15), ribosomal protein S15a (RPS15A), ribosomal protein S16 (RPS16), ribosomal protein S19 (RPS19), ribosomal protein S20 (RPS20), ribosomal protein S21 (RPS21), ribosomal protein S23 (RPS23), ribosomal protein S25 (RPS25), ribosomal protein S26 (RPS26), ribosomal protein S27 (RPS27), ribosomal protein S27a (RPS27a), ribosomal protein S28 (RPS28), ribosomal protein S29 (RPS29), ribosomal protein L15 (RPL15), ribosomal protein S2 (RPS2), ribosomal protein L14 (RPL14), ribosomal protein S14 (RPS14), ribosomal protein L10 (RPL10), ribosomal protein L10a (RPL10A), ribosomal protein L35 (RPL35), ribosomal protein L13a (RPL13A), ribosomal protein L36 (RPL36), ribosomal protein L36a (RPL36A), ribosomal protein L41 (RPL41), ribosomal protein S18 (RPS18), ribosomal protein S24 (RPS24), ribosomal protein L8 (RPL8), ribosomal protein L34 (RPL34), ribosomal protein S17 (RPS17), ribosomal protein SA (RPSA), ubiquitin A-52 residue ribosomal protein fusion product 1 (UBA52), Finkel-Biskis-Reilly murine sarcoma virus (FBR-MuSV) ubiquitously expressed (FAU), ribosomal protein L22-like 1 (RPL22L1), ribosomal protein S17 (RPS17), ribosomal protein L39-like (RPL39L), ribosomal protein L10-like (RPL10L), ribosomal protein L36a-like (RPL36AL), ribosomal protein L3-like (RPL3L), ribosomal protein S27-like (RPS27L), ribosomal protein L26-like 1 (RPL26L1), ribosomal protein L7-like 1 (RPL7L1), ribosomal protein L13a pseudogene (RPL13AP), ribosomal protein L37a pseudogene 8 (RPL37AP8), ribosomal protein S10 pseudogene 5 (RPS10P5), ribosomal protein S26 pseudogene 11 (RPS26P11), ribosomal protein L39 pseudogene 5 (RPL39P5), ribosomal protein, large, P0 pseudogene 6 (RPLP0P6) and ribosomal protein L36 pseudogene 14 (RPL36P14).

Preferably, the further 5′-UTR comprises or consists of a nucleic acid sequence which is derived from the 5′-UTR of a TOP gene or which is derived from a fragment, homolog or variant of the 5′-UTR of a TOP gene.

It is particularly preferred that the 5′-UTR element does not comprise a TOP-motif or a 5′TOP, as defined above. In particular, it is preferred that a 5′-UTR of a TOP gene is a 5′-UTR of a TOP gene lacking the TOP motif.

The nucleic acid sequence which is derived from the 5′-UTR of a TOP gene is derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the further 5′-UTR is preferably selected from 5′-UTR elements comprising or consisting of a nucleic acid sequence which is derived from a nucleic acid sequence selected from the group consisting of SEQ ID NOs. 1-1363, SEQ ID NO. 1395, SEQ ID NO. 1421 and SEQ ID NO. 1422 of the patent application WO2013/143700 whose disclosure is incorporated herein by reference, from the homologs of SEQ ID NOs. 1-1363, SEQ ID NO. 1395, SEQ ID NO. 1421 and SEQ ID NO. 1422 of the patent application WO2013/143700, from a variant thereof, or preferably from a corresponding RNA sequence. The term “homologs of SEQ ID NOs. 1-1363, SEQ ID NO. 1395, SEQ ID NO. 1421 and SEQ ID NO. 1422 of the patent application WO2013/143700” refers to sequences of other species than Homo sapiens, which are homologous to the sequences according to SEQ ID NOs. 1-1363, SEQ ID NO. 1395, SEQ ID NO. 1421 and SEQ ID NO. 1422 of the patent application WO2013/143700.

In a preferred embodiment, the further 5′-UTR comprises or consists of a nucleic acid sequence which is derived from a nucleic acid sequence extending from nucleotide position 5 (i.e. the nucleotide that is located at position 5 in the sequence) to the nucleotide position immediately 5′ to the start codon (located at the 3′ end of the sequences), e.g. the nucleotide position immediately 5′ to the ATG sequence, of a nucleic acid sequence selected from SEQ ID NOs. 1-1363, SEQ ID NO. 1395, SEQ ID NO. 1421 and SEQ ID NO. 1422 of the patent application WO2013/143700, from the homologs of SEQ ID NOs. 1-1363, SEQ ID NO. 1395, SEQ ID NO. 1421 and SEQ ID NO. 1422 of the patent application WO2013/143700, from a variant thereof, or a corresponding RNA sequence. It is particularly preferred that the further 5′-UTR is derived from a nucleic acid sequence extending from the nucleotide position immediately 3′ to the 5′TOP to the nucleotide position immediately 5′ to the start codon (located at the 3′ end of the sequences), e.g. the nucleotide position immediately 5′ to the ATG sequence, of a nucleic acid sequence selected from SEQ ID NOs. 1-1363, SEQ ID NO. 1395, SEQ ID NO. 1421 and SEQ ID NO. 1422 of the patent application WO2013/143700, from the homologs of SEQ ID NOs. 1-1363, SEQ ID NO. 1395, SEQ ID NO. 1421 and SEQ ID NO. 1422 of the patent application WO2013/143700, from a variant thereof, or a corresponding RNA sequence.

In a particularly preferred embodiment, the further 5′-UTR comprises or consists of a nucleic acid sequence which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence which is derived from a 5′-UTR of a nucleic acid sequence according to any of SEQ ID NOs: 170, 232, 244, 259, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1346, 1347, 1348, 1349, 1350, 1351, 1352, 1353, 1354, 1355, 1356, 1357, 1358, 1359, or 1360 of the patent application WO2013/143700, a corresponding RNA sequence, a homolog thereof, or a variant thereof as described herein, preferably lacking the 5′-TOP motif. As described above, the sequence extending from position 5 to the nucleotide immediately 5′ to the ATG (which is located at the 3′end of the sequences) corresponds to the 5′-UTR of said sequences.

Preferably, the further 5′-UTR comprises or consists of a nucleic acid sequence which is derived from a 5′-UTR of a TOP gene encoding a ribosomal Large protein (RPL) or from a homolog or variant of a 5′-UTR of a TOP gene encoding a ribosomal Large protein (RPL). For example, the 5′-UTR element comprises or consists of a nucleic acid sequence which is derived from a 5′-UTR of a nucleic acid sequence according to any of SEQ ID NOs: SEQ ID NOs: 67, 259, 1284-1318, 1344, 1346, 1348-1354, 1357, 1358, 1421 and 1422 of the patent application WO2013/143700, a corresponding RNA sequence, a homolog thereof, or a variant thereof as described herein, preferably lacking the 5′TOP motif.

In a particularly preferred embodiment, the 5′-UTR element comprises or consists of a nucleic acid sequence which is derived from the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, or from a variant of the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, wherein preferably the further 5′-UTR does not comprise the 5′TOP of said gene.

Accordingly, in a particularly preferred embodiment, the further 5′-UTR comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 33 (5′-UTR of human ribosomal protein Large 32 lacking the 5′ terminal oligopyrimidine tract: GGCGCTGCCTACGGAGGTGGCAGCCATCTCCTTCTCGGCATC (SEQ ID NO. 33); corresponding to SEQ ID NO. 1368 of the patent application WO2013/143700) or preferably to a corresponding RNA sequence, or wherein the further 5′-UTR comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 33 or more preferably to a corresponding RNA sequence, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In some embodiments, the artificial nucleic acid molecule comprises a further 5′-UTR which comprises or consists of a nucleic acid sequence which is derived from the 5′-UTR of a vertebrate TOP gene, such as a mammalian, e.g. a human TOP gene, selected from RPSA, RPS2, RPS3, RPS3A, RPS4, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS27A, RPS28, RPS29, RPS30, RPL3, RPL4, RPL5, RPL6, RPL7, RPL7A, RPL8, RPL9, RPL10, RPL10A, RPL11, RPL12, RPL13, RPL13A, RPL14, RPL15, RPL17, RPL18, RPL18A, RPL19, RPL21, RPL22, RPL23, RPL23A, RPL24, RPL26, RPL27, RPL27A, RPL28, RPL29, RPL30, RPL31, RPL32, RPL34, RPL35, RPL35A, RPL36, RPL36A, RPL37, RPL37A, RPL38, RPL39, RPL40, RPL41, RPLP0, RPLP1, RPLP2, RPLP3, RPLP0, RPLP1, RPLP2, EEF1A1, EEF1B2, EEF1D, EEF1G, EEF2, EIF3E, EIF3F, EIF3H, EIF2S3, EIF3C, EIF3K, EIF3EIP, EIF4A2, PABPC1, HNRNPA1, TPT1, TUBBI, UBA52, NPM1, ATP5G2, GNB2L1, NME2, UQCRB or from a homolog or variant thereof, wherein preferably the further 5′-UTR does not comprise a TOP-motif or the 5′TOP of said genes, and wherein optionally the further 5′-UTR starts at its 5′-end with a nucleotide located at position 1, 2, 3, 4, 5, 6, 7, 8, 9 or 10 downstream of the 5′terminal oligopyrimidine tract (TOP) and wherein further optionally the further 5′-UTR which is derived from a 5′-UTR of a TOP gene terminates at its 3′-end with a nucleotide located at position 1, 2, 3, 4, 5, 6, 7, 8, 9 or 10 upstream of the start codon (A(U/T)G) of the gene it is derived from.

The artificial nucleic acid molecule according to the present invention may be RNA, such as mRNA or viral RNA or a replicon, DNA, such as a DNA plasmid or viral DNA, or may be a modified RNA or DNA molecule. It may be provided as a double-stranded molecule having a sense strand and an anti-sense strand, for example, as a DNA molecule having a sense strand and an anti-sense strand.

The artificial nucleic acid molecule according to the present invention may further comprise optionally a 5′-cap. The optional 5′-cap is preferably located 5′ to the ORF, more preferably 5′ to the at least one 5′-UTR or to any further 5′-UTR within the artificial nucleic acid molecule according to the present invention.

Preferably, the artificial nucleic acid molecule according to the present invention further comprises a poly(A) sequence and/or a polyadenylation signal. Preferably, the optional poly(A) sequence is located 3′ to the at least one 3′-UTR element or to any further 3′-UTR, more preferably the optional poly(A) sequence is connected to the 3′-end of an 3′-UTR element. The connection may be direct or indirect, for example, via a stretch of 2, 4, 6, 8, 10, 20 etc. nucleotides, such as via a linker of 1-50, preferably of 1-20 nucleotides, e.g. comprising or consisting of one or more restriction sites. However, even if the artificial nucleic acid molecule according to the present invention does not comprise a 3′-UTR, for example if it only comprises at least one 5′-UTR element, it preferably still comprises a poly(A) sequence and/or a polyadenylation signal.

In one embodiment, the optional polyadenylation signal is located downstream of the 3′ of the 3′-UTR element. Preferably, the polyadenylation signal comprises the consensus sequence NN(U/T)ANA, with N=A or U, preferably AA(U/T)AAA or A(U/T)(U/T)AAA. Such consensus sequence may be recognised by most animal and bacterial cell-systems, for example by the polyadenylation-factors, such as cleavage/polyadenylation specificity factor (CPSF) cooperating with CstF, PAP, PAB2, CFI and/or CFII. Preferably, the polyadenylation signal, preferably the consensus sequence NNUANA, is located less than about 50 nucleotides, more preferably less than about 30 bases, most preferably less than about 25 bases, for example 21 bases, downstream of the 3′-end of the 3′-UTR element or of the ORF, if no 3′-UTR element is present.

Transcription of an artificial nucleic acid molecule according to the present invention, e.g. of an artificial DNA molecule, comprising a polyadenylation signal downstream of the 3′-UTR element (or of the ORF) will result in a premature-RNA containing the polyadenylation signal downstream of its 3′-UTR element (or of the ORF).

Using an appropriate transcription system will then lead to attachment of a poly(A) sequence to the premature-RNA. For example, the inventive artificial nucleic acid molecule may be a DNA molecule comprising a 3′-UTR element as described above and a polyadenylation signal, which may result in polyadenylation of an RNA upon transcription of this DNA molecule. Accordingly, a resulting RNA may comprise a combination of the inventive 3′-UTR element followed by a poly(A) sequence.

Potential transcription systems are in vitro transcription systems or cellular transcription systems etc. Accordingly, transcription of an artificial nucleic acid molecule according to the invention, e.g. transcription of an artificial nucleic acid molecule comprising an open reading frame, a 3′-UTR element and/or a 5′-UTR element and optionally a polyadenylation-signal, may result in an mRNA molecule comprising an open reading frame, a 3′-UTR element and optionally a poly(A) sequence.

Accordingly, the invention also provides an artificial nucleic acid molecule, which is an mRNA molecule comprising an open reading frame, a 3′-UTR element as described above and/or a 5′-UTR element as described above and optionally a poly(A) sequence.

In another embodiment, the 3′-UTR of the artificial nucleic acid molecule according to the invention does not comprise a polyadenylation signal or a poly(A) sequence. Further preferably, the artificial nucleic acid molecule according to the invention does not comprise a polyadenylation signal or a poly(A) sequence. More preferably, the 3′-UTR of the artificial nucleic acid molecule, or the inventive artificial nucleic acid molecule as such, does not comprise a polyadenylation signal, in particular it does not comprise the polyadenylation signal AAU/TAAA.

In a preferred embodiment, the invention provides an artificial nucleic acid molecule which is an artificial RNA molecule comprising an open reading frame and an RNA sequence corresponding to a DNA sequence selected from the group consisting of sequences according to SEQ ID NOs: 1 to 30, preferably from the group consisting of sequences according to SEQ ID NO. 1, SEQ ID NO. 5, SEQ ID NO. 8, SEQ ID NO. 11, SEQ ID NO. 13, SEQ ID NO. 14, SEQ ID NO. 17, SEQ ID NO. 20, SEQ ID NO. 25 and SEQ ID NO. 28, or sequences having an identity of at least about 40% or more to SEQ ID NOs: 1 to 30, preferably to SEQ ID NO. 1, SEQ ID NO. 5, SEQ ID NO. 8, SEQ ID NO. 11, SEQ ID NO. 13, SEQ ID NO. 14, SEQ ID NO. 17, SEQ ID NO. 20, SEQ ID NO. 25 and SEQ ID NO. 28 or a fragment thereof as described above. Moreover, a corresponding artificial DNA molecule is also provided.

In another preferred embodiment, the invention provides an artificial nucleic acid molecule which is an artificial DNA molecule comprising an open reading frame and a sequence selected from the group consisting of sequences according to SEQ ID NOs: 1 to 30, preferably from the group consisting of sequences according to SEQ ID NO. 1, SEQ ID NO. 5, SEQ ID NO. 8, SEQ ID NO. 11, SEQ ID NO. 13, SEQ ID NO. 14, SEQ ID NO. 17, SEQ ID NO. 20, SEQ ID NO. 25 and SEQ ID NO. 28, or sequences having an identity of at least about 40% or more to SEQ ID NOs: 1 to 30, preferably to SEQ ID NO. 1, SEQ ID NO. 5, SEQ ID NO. 8, SEQ ID NO. 11, SEQ ID NO. 13, SEQ ID NO. 14, SEQ ID NO. 17, SEQ ID NO. 20, SEQ ID NO. 25 and SEQ ID NO. 28.

Accordingly, the invention provides an artificial nucleic acid molecule which may serve as a template for an RNA molecule, preferably for an mRNA molecule, which is stabilised and optimized with respect to translation efficiency. In other words, the artificial nucleic acid molecule may be a DNA which may be used as a template for production of an mRNA. The obtainable mRNA, may, in turn, be translated for production of a desired peptide or protein encoded by the open reading frame. If the artificial nucleic acid molecule is a DNA, it may, for example, be used as a double-stranded storage form for continued and repetitive in vitro or in vivo production of mRNA. Thereby, in vitro refers in particular to (“living”) cells and/or tissue, including tissue of a living subject. Cells include in particular cell lines, primary cells, cells in tissue or subjects. In specific embodiments cell types allowing cell culture may be suitable for the present invention. Particularly preferred are mammalian cells, e.g. human cells and mouse cells. In particularly preferred embodiments the human cell lines HeLa, and U-937 and the mouse cell lines NIH3T3, JAWSII and L929 are used. Furthermore primary cells are particularly preferred, in particular preferred embodiments human dermal fibroblasts (HDF) may be used. Alternatively also a tissue of a subject may be used.

In one embodiment, the artificial nucleic acid molecule according to the present invention further comprises a poly(A) sequence. For example, a DNA molecule comprising an ORF, optionally followed by a 3′ UTR, may contain a stretch of thymidine nucleotides which can be transcribed into a poly(A) sequence in the resulting mRNA. The length of the poly(A) sequence may vary. For example, the poly(A) sequence may have a length of about 20 adenine nucleotides up to about 300 adenine nucleotides, preferably of about 40 to about 200 adenine nucleotides, more preferably from about 50 to about 100 adenine nucleotides, such as about 60, 70, 80, 90 or 100 adenine nucleotides. Most preferably, the inventive nucleic acid comprises a poly(A) sequence of about 60 to about 70 nucleotides, most preferably 64 adenine nucleotides.

Artificial RNA-molecules may also be obtainable in vitro by common methods of chemical-synthesis without being necessarily transcribed from a DNA-progenitor.

In a particularly preferred embodiment, the artificial nucleic acid molecule according to the present invention is an RNA molecule, preferably an mRNA molecule comprising in 5′-to-3′-direction an open reading frame, a 3′-UTR element as described above and a poly(A) sequence or comprising in 5′-to-3′-direction a 5′-UTR element as described above, an open reading frame and a poly(A) sequence.

In a preferred embodiment, the open reading frame is derived from a gene, which is distinct from the gene from which the 3′-UTR element and/or the 5′-UTR element of the inventive artificial nucleic acid is derived. In some further preferred embodiments, the open reading frame does not code for a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), Ybx1 (Y-Box binding protein 1), Ndufb8 (NADH dehydrogenase (ubiquinone) 1 beta subcomplex 8), CNTN1 (contactin 1), preferably CNTN1-004 or variants thereof, provided that the 3′-UTR element and/or the 5′-UTR element is a sequence which is selected from the group consisting of sequences according to SEQ ID NO. 1 to SEQ ID NO. 30.

In a preferred embodiment, the ORF does not encode human or plant, in particular Arabidopsis, ribosomal proteins, in particular does not encode human ribosomal protein S6 (RPS6), human ribosomal protein L36a-like (RPL36AL) or Arabidopsis ribosomal protein S16 (RPS16). In a further preferred embodiment, the open reading frame (ORF) does not encode ribosomal protein S6 (RPS6), ribosomal protein L36a-like (RPL36AL) or ribosomal protein S16 (RPS16) of whatever origin.

In one embodiment, the invention provides an artificial DNA molecule comprising an open reading frame, preferably an open reading frame derived from a gene, which is distinct from the gene from which the 3′-UTR element and/or the 5′-UTR element is derived; a 3′-UTR element comprising or consisting of a sequence which has at least about 60%, preferably at least about 70%, more preferably at least about 80%, more preferably at least about 90%, even more preferably at least about 95%; even more preferably at least 99%; even more preferably 100% sequence identity to a DNA sequence selected from the group consisting of sequences according to SEQ ID NO. 1 to 24, and/or a 5′-UTR element comprising or consisting of a sequence which has at least about 60%, preferably at least about 70%, more preferably at least about 80%, more preferably at least about 90%, even more preferably at least about 95%; even more preferably at least 99%; even more preferably 100% sequence identity to a DNA sequence selected from the group consisting of sequences according to SEQ ID NO. 25 to 30; and a polyadenylation signal and/or a poly(A) sequence. In a further embodiment there is provided a composition comprising a plurality of RNA molecules of the embodiments in pharmaceutically acceptable carrier, wherein at least about 90%, 91%, 92%, 93%, 94%, 95%, 96%, 97%, 98%, 99% or greater of the RNA in the composition comprises a poly(A) sequence that differs in length by no nor that 10 nucleotides. In a preferred embodiment at least about 90%, 91%, 92%, 93%, 94%, 95%, 96%, 97%, 98%, 99% or greater of the RNA in the composition comprises a poly(A) sequence of identical length. In certain embodiments, the poly(A) sequence is positioned at the 3′ end of the RNA, with no other nucleotides positioned 3′ relative the poly(A) sequence. In still a further embodiment, there is provided a composition comprising a plurality of RNA molecules of the embodiments in pharmaceutically acceptable carrier, wherein said plurality of RNA molecules comprise both capped and uncapped RNAs. For example, in some aspects, a composition comprises a plurality of RNA molecules wherein no more than 95%, 90%, 80%, 70% or 60% of the RNAs comprise a cap and the remaining RNA molecules are uncapped.

Furthermore, the invention provides an artificial RNA molecule, preferably an artificial mRNA molecule or an artificial viral RNA molecule, comprising an open reading frame, preferably an open reading frame is derived from a gene, which is distinct from the gene from which the 3′-UTR element and/or the 5′-UTR element is derived; a 3′-UTR element comprising or consisting of a sequence which has at least about 60%, preferably at least about 70%, more preferably at least about 80%, more preferably at least about 90%, even more preferably at least about 95%; even more preferably at least 99%; even more preferably 100% sequence identity to an RNA sequence corresponding to a DNA sequence selected from the group consisting of sequences according to SEQ ID NO. 1 to 24, and/or a 5′-UTR element comprising or consisting of a sequence which has at least about 60%, preferably at least about 70%, more preferably at least about 80%, more preferably at least about 90%, even more preferably at least about 95%; even more preferably at least 99%; even more preferably 100% sequence identity to an RNA sequence corresponding to a DNA sequence selected from the group consisting of sequences according to SEQ ID NO. 25 to 30; and a polyadenylation signal and/or a poly(A) sequence.

The invention provides an artificial nucleic acid molecule, preferably an artificial mRNA, which may be characterized by increased and/or prolonged expression of the encoded peptide or protein. Without being bound by any theory, enhanced stability of protein expression and thus prolonged protein expression may result from reduction in degradation of the artificial nucleic acid molecule, such as an artificial mRNA molecule according to the present invention. Accordingly, the inventive 3′-UTR element and/or the inventive 5′-UTR element may prevent the artificial nucleic acid from degradation and decay.

Preferably, the artificial nucleic acid molecule may additionally comprise a histone stem-loop. Thus, an artificial nucleic acid molecule according to the present invention may, for example, comprise in 5′-to-3′-direction an ORF, a 3′-UTR element, an optional histone stem-loop sequence, an optional poly(A) sequence or polyadenylation signal and an optional poly(C) sequence or in 5′-to-3′-direction an 5′-UTR element, an ORF, an optional histone stem-loop sequence, an optional poly(A) sequence or polyadenylation signal and an optional poly(C) sequence or in 5′-to-3′-direction an 5′-UTR element, an ORF, a 3′-UTR element, an optional histone stem-loop sequence, an optional poly(A) sequence or polyadenylation signal and an optional poly(C) sequence. It may also comprise in 5′-to-3′-direction an ORF, an 3′-UTR element, an optional poly(A) sequence, an optional poly (C) sequence and an optional histone stem-loop sequence, or in 5′-to-3′-direction an 5′-UTR element, an ORF, an optional poly(A) sequence, an optional poly(C) sequence and an optional histone stem-loop sequence, or in 5′-to-3′-direction an 5′-UTR element, an ORF, a 3′-UTR element, an optional poly(A) sequence, an optional poly(C) sequence and an optional histone stem-loop sequence.

In a preferred embodiment, the artificial nucleic acid molecule according to the invention further comprises at least one histone stem-loop sequence.

Such histone stem-loop sequences are preferably selected from histone stem-loop sequences as disclosed in WO 2012/019780, whose disclosure is incorporated herewith by reference.

A histone stem-loop sequence, suitable to be used within the present invention, is preferably selected from at least one of the following formulae (I) or (II):

formula (I) (stem-loop sequence without stem bordering elements):

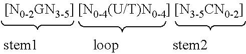

formula (II) (stem-loop sequence with stem bordering elements):

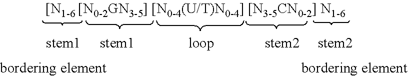

wherein:
stem1 or stem2 bordering elements N₁₋₆ is a consecutive sequence of 1 to 6, preferably of 2 to 6, more preferably of 2 to 5, even more preferably of 3 to 5, most preferably of 4 to 5 or 5 N, wherein each N is independently from another selected from a nucleotide selected from A, U, T, G and C, or a nucleotide analogue thereof;
stem1 [No₂GN₃-5] is reverse complementary or partially reverse complementary with element stem2, and is a consecutive sequence between of 5 to 7 nucleotides;
   wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof, and
   wherein G is guanosine or an analogue thereof, and may be optionally replaced by a cytidine or an analogue thereof, provided that its complementary nucleotide cytidine in stem2 is replaced by guanosine;
loop sequence [No₄(U/T)No₄] is located between elements stem1 and stem2, and is a consecutive sequence of 3 to 5 nucleotides, more preferably of 4 nucleotides;
   wherein each N₀₋₄ is independent from another a consecutive sequence of 0 to 4, preferably of 1 to 3, more preferably of 1 to 2 N, wherein each N is independently from another selected from a nucleotide selected from A, U, T, G and C or a nucleotide analogue thereof; and wherein U/T represents uridine, or optionally thymidine;
stem2 [N3m₅CNo₂] is reverse complementary or partially reverse complementary with element stem1, and is a consecutive sequence between of 5 to 7 nucleotides;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof;
   wherein N₀-2 is a consecutive sequence of 0 to 2, preferably of 0 to 1, more preferably of 1 N, wherein each N is independently from another selected from a nucleotide selected from A, U, T, G or C or a nucleotide analogue thereof; and
   wherein C is cytidine or an analogue thereof, and may be optionally replaced by a guanosine or an analogue thereof provided that its complementary nucleoside guanosine in stem1 is replaced by cytidine;
      wherein
      stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one ore more bases in one stem do not have a complementary base in the reverse complementary sequence of the other stem.

According to a further preferred embodiment the histone stem-loop sequence may be selected according to at least one of the following specific formulae (Ia) or (IIa):

formula (Ia) (stem-loop sequence without stem bordering elements):

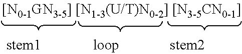

formula (IIa) (stem-loop sequence with stem bordering elements):

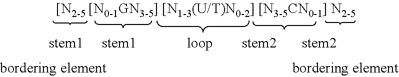

wherein:
N, C, G, T and U are as defined above.

According to a further more particularly preferred embodiment of the first aspect, the artificial nucleic acid molecule sequence may comprise at least one histone stem-loop sequence according to at least one of the following specific formulae (Ib) or (IIb):

formula (Ib) (stem-loop sequence without stem bordering elements):

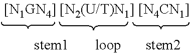

formula (IIb) (stem-loop sequence with stem bordering elements):

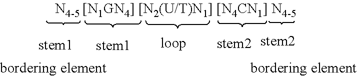

wherein:
N, C, G, T and U are as defined above.

A particular preferred histone stem-loop sequence is the sequence according to SEQ ID NO: 34: CAAAGGCTCTTTTCAGAGCCACCA or more preferably the corresponding RNA sequence of the nucleic acid sequence according to SEQ ID NO: 34.

As an example, the single elements may be present in the artificial nucleic acid molecule in the following order:
5′-cap-5′-UTR (element)-ORF-3′-UTR (element)-histone stem-loop-poly(A)/(C) sequence;
5′-cap-5′-UTR (element)-ORF-3′-UTR (element)-poly(A)/(C) sequence-histone stem-loop;
5′-cap-5′-UTR (element)-ORF-IRES-ORF-3′-UTR (element)-histone stem-loop-poly(A)/(C) sequence;
5′-cap-5′-UTR (element)-ORF-IRES-ORF-3′-UTR (element)-histone stem-loop-poly(A)/(C) sequence-poly(A)/(C) sequence;
5′-cap-5′-UTR (element)-ORF-IRES-ORF-3′-UTR (element)-poly(A)/(C) sequence-histone stem-loop;
5′-cap-5′-UTR (element)-ORF-IRES-ORF-3′-UTR (element)-poly(A)/(C) sequence-poly(A)/(C) sequence-histone stem-loop;
5′-cap-5′-UTR (element)-ORF-3′-UTR (element)-poly(A)/(C) sequence-poly(A)/(C) sequence;
5′-cap-5′-UTR (element)-ORF-3′-UTR (element)-poly(A)/(C) sequence-poly(A)/(C) sequence-histone stem loop; etc.

In some embodiments, the artificial nucleic acid molecule comprises further elements such as a 5′-cap, a poly(C) sequence and/or an IRES-motif. A 5′-cap may be added during transcription or posttranscriptionally to the 5′end of an RNA. Furthermore, the inventive artificial nucleic acid molecule, particularly if the nucleic acid is in the form of an mRNA or codes for an mRNA, may be modified by a sequence of at least 10 cytidines, preferably at least 20 cytidines, more preferably at least 30 cytidines (so-called “poly(C) sequence”). In particular, the inventive artificial nucleic acid molecule may contain, especially if the nucleic acid is in the form of an (m)RNA or codes for an mRNA, a poly(C) sequence of typically about 10 to 200 cytidine nucleotides, preferably about 10 to 100 cytidine nucleotides, more preferably about 10 to 70 cytidine nucleotides or even more preferably about 20 to 50 or even 20 to 30 cytidine nucleotides. Most preferably, the inventive nucleic acid comprises a poly(C) sequence of 30 cytidine residues. Thus, preferably the artificial nucleic acid molecule according to the present invention comprises, preferably in 5′-to-3′ direction, at least one 5′-UTR element as described above, an ORF, at least one 3′-UTR element as described above, a poly(A) sequence or a polyadenylation signal, and a poly(C) sequence or, in 5′-to-3′ direction, optionally a further 5′-UTR, an ORF, at least one 3′-UTR element as described above, a poly(A) sequence or a polyadenylation signal, and a poly(C) sequence, or, in 5′-to-3′ direction, at least one 5′-UTR element as described above, an ORF, optionally a further 3′-UTR, a poly(A) sequence or a polyadenylation signal, and a poly(C) sequence.

An internal ribosome entry site (IRES) sequence or IRES-motif may separate several open reading frames, for example if the artificial nucleic acid molecule encodes for two or more peptides or proteins. An IRES-sequence may be particularly helpful if the artificial nucleic acid molecule is a bi- or multicistronic nucleic acid molecule.

Furthermore, the artificial nucleic acid molecule may comprise additional 5′-elements, preferably a promoter or a promoter containing-sequence. The promoter may drive and or regulate transcription of the artificial nucleic acid molecule according to the present invention, for example of an artificial DNA-molecule according to the present invention.

Preferably, the artificial nucleic acid molecule according to the present invention, preferably the open reading frame, is at least partially G/C modified. Thus, the inventive artificial nucleic acid molecule may be thermodynamically stabilized by modifying the G (guanosine)/C (cytidine) content of the molecule. The G/C content of the open reading frame of an artificial nucleic acid molecule according to the present invention may be increased compared to the G/C content of the open reading frame of a corresponding wild type sequence, preferably by using the degeneration of the genetic code. Thus, the encoded amino acid sequence of the artificial nucleic acid molecule is preferably not modified by the G/C modification compared to the coded amino acid sequence of the particular wild type sequence. The codons of the coding sequence or the whole artificial nucleic acid molecule, e.g. an mRNA, may therefore be varied compared to the wild type coding sequence, such that they include an increased amount of G/C nucleotides while the translated amino acid sequence is maintained. Due to the fact that several codons code for one and the same amino acid (so-called degeneration of the genetic code), it is feasible to alter codons while not altering the encoded peptide/protein sequence (so-called alternative codon usage). Hence, it is possible to specifically introduce certain codons (in exchange for the respective wild-type codons encoding the same amino acid), which are more favourable with respect to stability of RNA and/or with respect to codon usage in a subject (so-called codon optimization).

Depending on the amino acid to be encoded by the coding region of the inventive artificial nucleic acid molecule as defined herein, there are various possibilities for modification of the nucleic acid sequence, e.g. the open reading frame, compared to its wild type coding region. In the case of amino acids, which are encoded by codons which contain exclusively G or C nucleotides, no modification of the codon is necessary. Thus, the codons for Pro (CCC or CCG), Arg (CGC or CGG), Ala (GCC or GCG) and Gly (GGC or GGG) require no modification, since no A or U/T is present.

In contrast, codons which contain A and/or U/T nucleotides may be modified by substitution of other codons which code for the same amino acids but contain no A and/or U/T. For example
the codons for Pro can be modified from CC(U/T) or CCA to CCC or CCG;
the codons for Arg can be modified from CG(U/T) or CGA or AGA or AGG to CGC or CGG;
the codons for Ala can be modified from GC(U/T) or GCA to GCC or GCG;
the codons for Gly can be modified from GG(U/T) or GGA to GGC or GGG.

In other cases, although A or (U/T) nucleotides cannot be eliminated from the codons, it is however possible to decrease the A and (U/T) content by using codons which contain a lower content of A and/or (U/T) nucleotides. Examples of these are:

The codons for Phe can be modified from (U/T)(U/T)(U/T) to (U/T) (U/T)C;
the codons for Leu can be modified from (U/T) (U/T)A, (U/T) (U/T)G, C(U/T) (U/T) or C(U/T)A to C(U/T)C or C(U/T)G;
the codons for Ser can be modified from (U/T)C(U/T) or (U/T)CA or AG(U/T) to (U/T)CC, (U/T)CG or AGC;
the codon for Tyr can be modified from (U/T)A(U/T) to (U/T)AC;
the codon for Cys can be modified from (U/T)G(U/T) to (U/T)GC;
the codon for His can be modified from CA(U/T) to CAC;
the codon for Gln can be modified from CAA to CAG;
the codons for Ile can be modified from A(U/T)(U/T) or A(U/T)A to A(U/T)C;
the codons for Thr can be modified from AC(U/T) or ACA to ACC or ACG;
the codon for Asn can be modified from AA(U/T) to AAC;
the codon for Lys can be modified from AAA to AAG;
the codons for Val can be modified from G(U/T)(U/T) or G(U/T)A to G(U/T)C or G(U/T)G;
the codon for Asp can be modified from GA(U/T) to GAC;
the codon for Glu can be modified from GAA to GAG;
the stop codon (U/T)AA can be modified to (U/T)AG or (U/T)GA.

In the case of the codons for Met (A(U/T)G) and Trp ((U/T)GG), on the other hand, there is no possibility of sequence modification without altering the encoded amino acid sequence.

The substitutions listed above can be used either individually or in all possible combinations to increase the G/C content of the open reading frame of the inventive artificial nucleic acid molecule as defined herein, compared to its particular wild type open reading frame (i.e. the original sequence). Thus, for example, all codons for Thr occurring in the wild type sequence can be modified to ACC (or ACG).

Preferably, the G/C content of the open reading frame of the inventive artificial nucleic acid molecule as defined herein is increased by at least 7%, more preferably by at least 15%, particularly preferably by at least 20%, compared to the G/C content of the wild type coding region without altering the encoded amino acid sequence, i.e. using the degeneracy of the genetic code. According to a specific embodiment at least 5%, 10%, 20%, 30%, 40%, 50%, 60%, more preferably at least 70%, even more preferably at least 80% and most preferably at least 90%, 95% or even 100% of the substitutable codons in the open reading frame of the inventive artificial nucleic acid molecule or a fragment, variant or derivative thereof are substituted, thereby increasing the G/C content of said open reading frame.

In this context, it is particularly preferable to increase the G/C content of the open reading frame of the inventive artificial nucleic acid molecule as defined herein, to the maximum (i.e. 100% of the substitutable codons), compared to the wild type open reading frame, without altering the encoded amino acid sequence.

Furthermore, the open reading frame is preferably at least partially codon-optimized. Codon-optimization is based on the finding that the translation efficiency may be determined by a different frequency in the occurrence of transfer RNAs (tRNAs) in cells. Thus, if so-called “rare codons” are present in the coding region of the inventive artificial nucleic acid molecule as defined herein, to an increased extent, the translation of the corresponding modified nucleic acid sequence is less efficient than in the case where codons coding for relatively “frequent” tRNAs are present.

Thus, the open reading frame of the inventive artificial nucleic acid molecule is preferably modified compared to the corresponding wild type coding region such that at least one codon of the wild type sequence which codes for a tRNA which is relatively rare in the cell is exchanged for a codon which codes for a tRNA which is comparably frequent in the cell and carries the same amino acid as the relatively rare tRNA. By this modification, the open reading frame of the inventive artificial nucleic acid molecule as defined herein, is modified such that codons for which frequently occurring tRNAs are available may replace codons which correspond to rare tRNAs. In other words, according to the invention, by such a modification all codons of the wild type open reading frame which code for a rare tRNA may be exchanged for a codon which codes for a tRNA which is more frequent in the cell and which carries the same amino acid as the rare tRNA. Which tRNAs occur relatively frequently in the cell and which, in contrast, occur relatively rarely is known to a person skilled in the art; cf. e.g. Akashi, Curr. Opin. Genet. Dev. 2001, 11(6): 660-666. Accordingly, preferably, the open reading frame is codon-optimized, preferably with respect to the system in which the artificial nucleic acid molecule according to the present invention is to be expressed, preferably with respect to the system in which the artificial nucleic acid molecule according to the present invention is to be translated. Preferably, the codon usage of the open reading frame is codon-optimized according to mammalian codon usage, more preferably according to human codon usage. Preferably, the open reading frame is codon-optimized and G/C-content modified.

For further improving degradation resistance, e.g. resistance to in vivo (or in vitro as defined above) degradation by an exo- or endonuclease, and/or for further improving stability of protein expression from the artificial nucleic acid molecule according to the present invention, the artificial nucleic acid molecule may further comprise modifications, such as backbone modifications, sugar modifications and/or base modifications, e.g., lipid-modifications or the like. Preferably, the transcription and/or the translation of the artificial nucleic acid molecule according to the present invention is not significantly impaired by said modifications.

Generally, the artificial nucleic acid molecule of the present invention may comprise any native (=naturally occurring) nucleotide, e.g. guanosine, uracil, adenosine, and/or cytosine or an analogue thereof. In this respect, nucleotide analogues are defined as natively and non-natively occurring variants of the naturally occurring nucleotides adenosine, cytosine, thymidine, guanosine and uridine. Accordingly, analogues are e.g. chemically derivatized nucleotides with non-natively occurring functional groups, which are preferably added to or deleted from the naturally occurring nucleotide or which substitute the naturally occurring functional groups of a nucleotide. Accordingly, each component of the naturally occurring nucleotide may be modified, namely the base component, the sugar (ribose) component and/or the phosphate component forming the backbone (see above) of the RNA sequence. Analogues of guanosine, uridine, adenosine, thymidine and cytosine include, without implying any limitation, any natively occurring or non-natively occurring guanosine, uridine, adenosine, thymidine or cytosine that has been altered e.g. chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-Amino-2′-deoxyadenosine, 2′-Amino-2′-deoxycytidine, 2′-Amino-2′-deoxyguanosine, 2′-Amino-2′-deoxyuridine, 2-Amino-6-chloropurineriboside, 2-Aminopurine-riboside, 2′-Araadenosine, 2′-Aracytidine, 2′-Arauridine, 2′-Azido-2′-deoxyadenosine, 2′-Azido-2′-deoxycytidine, 2′-Azido-2′-deoxyguanosine, 2′-Azido-2′-deoxyuridine, 2-Chloroadenosine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-Fluorothymidine, 2-methyl-adenosine, 2-methyl-guanosine, 2-methyl-thio-N6-isopenenyl-adenosine, 2′-O-Methyl-2-aminoadenosine, 2′-O-Methyl-2′-deoxyadenosine, 2′-O-Methyl-2′-deoxycytidine, 2′-O-Methyl-2′-deoxyguanosine, 2′-O-Methyl-2′-deoxyuridine, 2′-O-Methyl-5-methyluridine, 2′-O-Methylinosine, 2′-O-Methylpseudouridine, 2-Thiocytidine, 2-thio-cytosine, 3-methyl-cytosine, 4-acetyl-cytosine, 4-Thiouridine, 5-(carboxyhydroxymethyl)-uracil, 5,6-Dihydrouridine, 5-Aminoallylcytidine, 5-Aminoallyl-deoxy-uridine, 5-Bromouridine, 5-carboxymehtylaminomethyl-2-thio-uracil, 5-carboxymethylamonomethyl-uracil, 5-Chloro-Ara-cytosine, 5-Fluoro-uridine, 5-Iodouridine, 5-methoxycarbonylmethyl-uridine, 5-methoxy-uridine, 5-methyl-2-thio-uridine, 6-Azacytidine, 6-Azauridine, 6-Chloro-7-deaza-guanosine, 6-Chloropurineriboside, 6-Mercapto-guanosine, 6-Methyl-mercaptopurine-riboside, 7-Deaza-2′-deoxy-guanosine, 7-Deazaadenosine, 7-methyl-guanosine, 8-Azaadenosine, 8-Bromo-adenosine, 8-Bromo-guanosine, 8-Mercapto-guanosine, 8-Oxoguanosine, Benzimidazole-riboside, Beta-D-mannosyl-queosine, Dihydro-uracil, Inosine, N1-Methyladenosine, N6-([6-Aminohexyl]carbamoylmethyl)-adenosine, N6-isopentenyl-adenosine, N6-methyl-adenosine, N7-Methyl-xanthosine, N-uracil-5-oxyacetic acid methyl ester, Puromycin, Queosine, Uracil-5-oxyacetic acid, Uracil-5-oxyacetic acid methyl ester, Wybutoxosine, Xanthosine, and Xylo-adenosine. The preparation of such analogues is known to a person skilled in the art, for example from U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642. In the case of an analogue as described above, particular preference may be given according to certain embodiments of the invention to those analogues that increase the protein expression of the encoded peptide or protein or that increase the immunogenicity of the artificial nucleic acid molecule of the invention and/or do not interfere with a further modification of the artificial nucleic acid molecule that has been introduced.

According to a particular embodiment, the artificial nucleic acid molecule of the present invention can contain a lipid modification.

In a preferred embodiment, the artificial nucleic acid molecule comprises, preferably from 5′ to 3′ direction, the following elements:
a 5′-UTR element which prolongs and/or increases protein production from said artificial nucleic acid molecule, preferably from a nucleic acid sequence according to any of SEQ ID NO: 25 to 30 and SEQ ID NOs: 319 to 382, more preferably of the 5′-UTR of MP68 or NDUFA4; or a further 5′-UTR, preferably a 5′-TOP UTR;
at least one open reading frame (ORF), wherein the ORF preferably comprises at least one modification with respect to the wild type sequence;
a 3′-UTR element which prolongs and/or increases protein production from said artificial nucleic acid molecule, preferably from a nucleic acid sequence according to any of SEQ ID NO: 1 to 24 and SEQ ID NOs: 49 to 318, more preferably of the 3′-UTR of GNAS, MORN2, GSTM1, NDUFA1, CBR2, YBX1, NDUFB8, or CNTN1; or a further 3′-UTR, preferably an albumin7 3′-UTR;
a poly(A) sequence, preferably comprising 64 adenylates;
a poly(C) sequence, preferably comprising 30 cytidylates;
a histone stem-loop sequence.

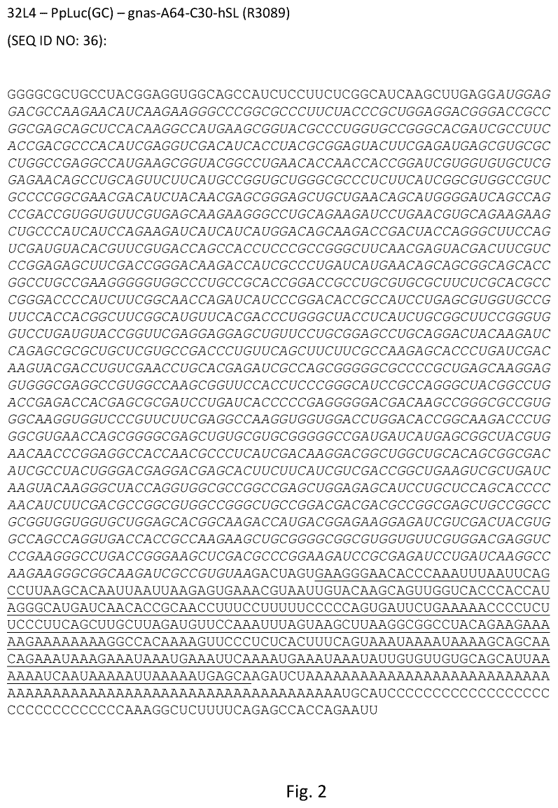

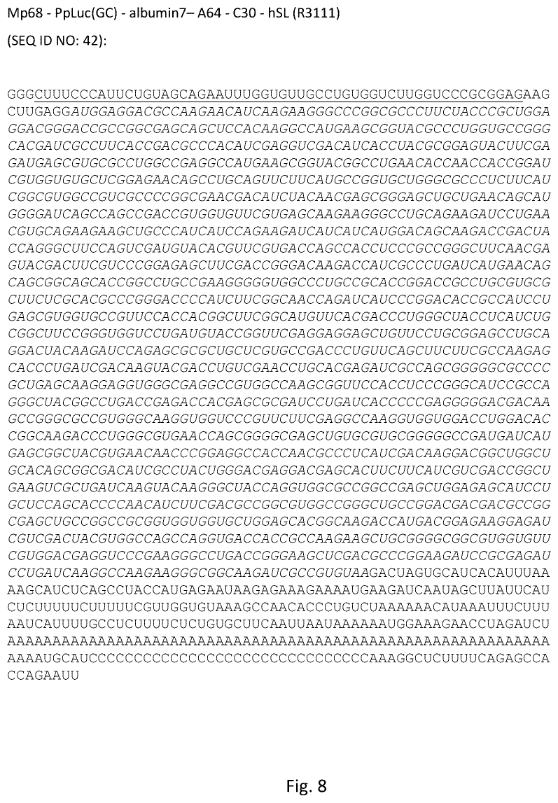

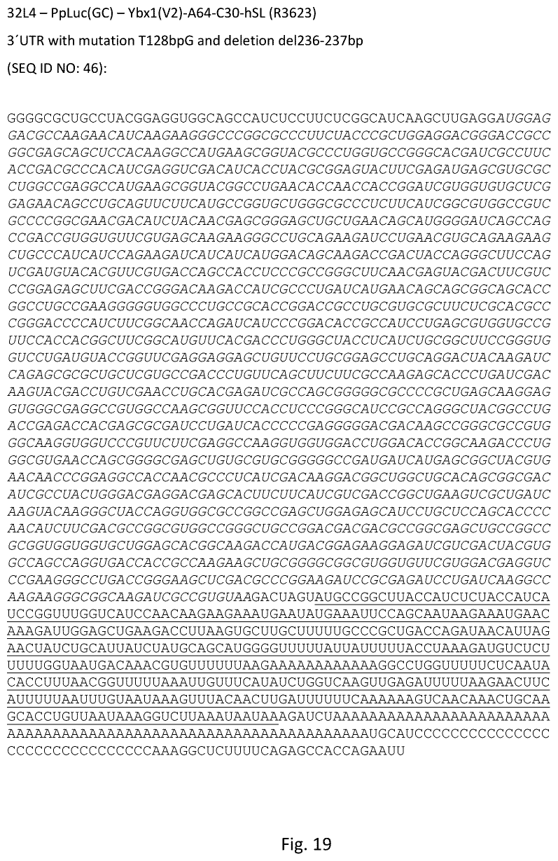

In another preferred embodiment, the artificial nucleic acid molecule comprises or consists of a nucleotide sequence selected from the group consisting of nucleic acid sequences according to SEQ ID NOs: 36 to 40, SEQ ID NOs: 42 and 43, SEQ ID NOs: 45 to 48, and SEQ ID NOs: 384 to 388 (see FIG. 2 to 6, FIG. 8, 9, 11, FIG. 19 to 21 and FIG. 26 to 30) or the complementary DNA sequence.

In a particularly preferred embodiment, the artificial nucleic acid molecule according to the invention may further comprise one or more of the modifications described in the following:

### Chemical Modifications:

The term “modification” as used herein with regard to the artificial nucleic acid molecule may refer to chemical modifications comprising backbone modifications as well as sugar modifications or base modifications.

In this context, the artificial nucleic acid molecule, preferably an RNA molecule, as defined herein may contain nucleotide analogues/modifications, e.g. backbone modifications, sugar modifications or base modifications. A backbone modification in connection with the present invention is a modification, in which phosphates of the backbone of the nucleotides contained in a nucleic acid molecule as defined herein are chemically modified. A sugar modification in connection with the present invention is a chemical modification of the sugar of the nucleotides of the nucleic acid molecule as defined herein. Furthermore, a base modification in connection with the present invention is a chemical modification of the base moiety of the nucleotides of the nucleic acid molecule of the nucleic acid molecule. In this context, nucleotide analogues or modifications are preferably selected from nucleotide analogues which are applicable for transcription and/or translation.

### Sugar Modifications:

The modified nucleosides and nucleotides, which may be incorporated into the artificial nucleic acid molecule, preferably an RNA, as described herein, can be modified in the sugar moiety. For example, the 2′ hydroxyl group (OH) of an RNA molecule can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), -0(CH2CH2o)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified nucleic acid molecule can include nucleotides containing, for instance, arabinose as the sugar.

### Backbone Modifications:

The phosphate backbone may further be modified in the modified nucleosides and nucleotides, which may be incorporated into the artificial nucleic acid molecule, preferably an RNA, as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Base Modifications:

The modified nucleosides and nucleotides, which may be incorporated into the artificial nucleic acid molecule, preferably an RNA molecule, as described herein, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

In specific embodiments, a modified nucleoside is 5′-0-(1-Thiophosphate)-Adenosine, 5′-0-(1-Thiophosphate)-Cytidine, 5′-0-(1-Thiophosphate)-Guanosine, 5′-0-(1-Thiophosphate)-Uridine or 5′-0-(1-Thiophosphate)-Pseudouridine.

In further specific embodiments the artificial nucleic acid molecule, preferably an RNA molecule, may comprise nucleoside modifications selected from 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

### Lipid Modification:

According to a further embodiment, the artificial nucleic acid molecule, preferably an RNA, as defined herein can contain a lipid modification. Such a lipid-modified RNA typically comprises an RNA as defined herein. Such a lipid-modified RNA molecule as defined herein typically further comprises at least one linker covalently linked with that RNA molecule, and at least one lipid covalently linked with the respective linker. Alternatively, the lipid-modified RNA molecule comprises at least one RNA molecule as defined herein and at least one (bifunctional) lipid covalently linked (without a linker) with that RNA molecule. According to a third alternative, the lipid-modified RNA molecule comprises an artificial nucleic acid molecule, preferably an RNA molecule, as defined herein, at least one linker covalently linked with that RNA molecule, and at least one lipid covalently linked with the respective linker, and also at least one (bifunctional) lipid covalently linked (without a linker) with that RNA molecule. In this context, it is particularly preferred that the lipid modification is present at the terminal ends of a linear RNA sequence.

### Modification of the 5′-End of the Modified RNA:

According to another preferred embodiment of the invention, the artificial nucleic acid molecule, preferably an RNA molecule, as defined herein, can be modified by the addition of a so-called “5′ CAP” structure. A 5-cap is an entity, typically a modified nucleotide entity, which generally “caps” the 5′-end of a mature mRNA. A 5-cap may typically be formed by a modified nucleotide, particularly by a derivative of a guanine nucleotide. Preferably, the 5′-cap is linked to the 5′-terminus via a 5-5-triphosphate linkage. A 5′-cap may be methylated, e.g. m7GpppN, wherein N is the terminal 5′ nucleotide of the nucleic acid carrying the 5′-cap, typically the 5′-end of an RNA. m7GpppN is the 5′-CAP structure which naturally occurs in mRNA transcribed by polymerase II and is therefore not considered as modification comprised in the modified RNA according to the invention. This means the artificial nucleic acid molecule, preferably an RNA molecule, according to the present invention may comprise a m7GpppN as 5′-CAP, but additionally the artificial nucleic acid molecule, preferably an RNA molecule, comprises at least one further modification as defined herein.

Further examples of 5′cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures are regarded as at least one modification comprised in the artificial nucleic acid molecule, preferably in an RNA molecule, according to the present invention.

Particularly preferred modified 5′-CAP structures are CAP1 (methylation of the ribose of the adjacent nucleotide of m7G), CAP2 (methylation of the ribose of the 2^{nd} nucleotide downstream of the m7G), CAP3 (methylation of the ribose of the 3^{rd} nucleotide downstream of the m7G), CAP4 (methylation of the ribose of the 4^{th} nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In a preferred embodiment, the at least one open reading frame encodes a therapeutic protein or peptide. In another embodiment, an antigen is encoded by the at least one open reading frame, such as a pathogenic antigen, a tumour antigen, an allergenic antigen or an autoimmune antigen. Therein, the administration of the artificial nucleic acid molecule encoding the antigen is used in a genetic vaccination approach against a disease involving said antigen.

In an alternative embodiment, an antibody or an antigen-specific T cell receptor or a fragment thereof is encoded by the at least one open reading frame of the artificial nucleic acid molecule according to the invention.

### Antigens:

### Pathogenic Antigens:

The artificial nucleic acid molecule according to the present invention may encode a protein or a peptide, which comprises a pathogenic antigen or a fragment, variant or derivative thereof. Such pathogenic antigens are derived from pathogenic organisms, in particular bacterial, viral or protozoological (multicellular) pathogenic organisms, which evoke an immunological reaction in a subject, in particular a mammalian subject, more particularly a human. More specifically, pathogenic antigens are preferably surface antigens, e.g. proteins (or fragments of proteins, e.g. the exterior portion of a surface antigen) located at the surface of the virus or the bacterial or protozoological organism.

Pathogenic antigens are peptide or protein antigens preferably derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, 0111 and 0104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In this context particularly preferred are antigens from the pathogens selected from Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

### Tumour Antigens:

In a further embodiment the artificial nucleic acid molecule according to the present invention may encode a protein or a peptide, which comprises a peptide or protein comprising a tumour antigen, a fragment, variant or derivative of said tumour antigen, preferably, wherein the tumour antigen is a melanocyte-specific antigen, a cancer-testis antigen or a tumour-specific antigen, preferably a CT-X antigen, a non-X CT-antigen, a binding partner for a CT-X antigen or a binding partner for a non-X CT-antigen or a tumour-specific antigen, more preferably a CT-X antigen, a binding partner for a non-X CT-antigen or a tumour-specific antigen or a fragment, variant or derivative of said tumour antigen; and wherein each of the nucleic acid sequences encodes a different peptide or protein; and wherein at least one of the nucleic acid sequences encodes for 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R17I, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-Fi, MAGE-H1, MAGEL2, mammaglobin A, MART-1/melan-A, MART-2, MART-2/m, matrix protein 22, MC1R, M-CSF, MEl/m, mesothelin, MG50/PXDN, MMPi1, MN/CA IX-antigen, MRP-3, MUC-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WTi and a immunoglobulin idiotype of a lymphoid blood cell or a T cell receptor idiotype of a lymphoid blood cell, or a fragment, variant or derivative of said tumour antigen; preferably survivin or a homologue thereof, an antigen from the MAGE-family or a binding partner thereof or a fragment, variant or derivative of said tumour antigen. Particularly preferred in this context are the tumour antigens NY-ESO-1, 5T4, MAGE-C1, MAGE-C2, Survivin, Muc-1, PSA, PSMA, PSCA, STEAP and PAP.

In a preferred embodiment, the artificial nucleic acid molecule encodes a protein or a peptide, which comprises a therapeutic protein or a fragment, variant or derivative thereof.

Therapeutic proteins as defined herein are peptides or proteins, which are beneficial for the treatment of any inherited or acquired disease or which improves the condition of an individual. Particularly, therapeutic proteins play an important role in the creation of therapeutic agents that could modify and repair genetic errors, destroy cancer cells or pathogen infected cells, treat immune system disorders, treat metabolic or endocrine disorders, among other functions. For instance, Erythropoietin (EPO), a protein hormone can be utilized in treating patients with erythrocyte deficiency, which is a common cause of kidney complications. Furthermore adjuvant proteins, therapeutic antibodies are encompassed by therapeutic proteins and also hormone replacement therapy which is e.g. used in the therapy of women in menopause. In more recent approaches, somatic cells of a patient are used to reprogram them into pluripotent stem cells, which replace the disputed stem cell therapy. Also these proteins used for reprogramming of somatic cells or used for differentiating of stem cells are defined herein as therapeutic proteins. Furthermore, therapeutic proteins may be used for other purposes, e.g. wound healing, tissue regeneration, angiogenesis, etc. Furthermore, antigen-specific B cell receptors and fragments and variants thereof are defined herein as therapeutic proteins.

Therefore therapeutic proteins can be used for various purposes including treatment of various diseases like e.g. infectious diseases, neoplasms (e.g. cancer or tumour diseases), diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired.

In this context, particularly preferred therapeutic proteins which can be used inter alia in the treatment of metabolic or endocrine disorders are selected from (in brackets the particular disease for which the therapeutic protein is used in the treatment): Acid sphingomyelinase (Niemann-Pick disease), Adipotide (obesity), Agalsidase-beta (human galactosidase A) (Fabry disease; prevents accumulation of lipids that could lead to renal and cardiovascular complications), Alglucosidase (Pompe disease (glycogen storage disease type II)), alpha-galactosidase A (alpha-GAL A, Agalsidase alpha) (Fabry disease), alpha-glucosidase (Glycogen storage disease (GSD), Morbus Pompe), alpha-L-iduronidase (mucopolysaccharidoses (MPS), Hurler syndrome, Scheie syndrome), alpha-N-acetylglucosaminidase (Sanfilippo syndrome), Amphiregulin (cancer, metabolic disorder), Angiopoietin ((Ang1, Ang2, Ang3, Ang4, ANGPTL2, ANGPTL3, ANGPTL4, ANGPTL5, ANGPTL6, ANGPTL7) (angiogenesis, stabilize vessels), Betacellulin (metabolic disorder), Beta-glucuronidase (Sly syndrome), Bone morphogenetic protein BMPs (BMP1, BMP2, BMP3, BMP4, BMP5, BMP6, BMP7, BMP8a, BMP8b, BMP10, BMP15) (regenerative effect, bone-related conditions, chronic kidney disease (CKD)), CLN6 protein (CLN6 disease Atypical Late Infantile, Late Onset variant, Early Juvenile, Neuronal Ceroid Lipofuscinoses (NCL)), Epidermal growth factor (EGF) (wound healing, regulation of cell growth, proliferation, and differentiation), Epigen (metabolic disorder), Epiregulin (metabolic disorder), Fibroblast Growth Factor (FGF, FGF-1, FGF-2, FGF-3, FGF-4, FGF-5, FGF-6, FGF-7, FGF-8, FGF-9, FGF-10, FGF-11, FGF-12, FGF-13, FGF-14, FGF-16, FGF-17, FGF-17, FGF-18, FGF-19, FGF-20, FGF-21, FGF-22, FGF-23) (wound healing, angiogenesis, endocrine disorders, tissue regeneration), Galsulphase (Mucopolysaccharidosis VI), Ghrelin (irritable bowel syndrome (IBS), obesity, Prader-Willi syndrome, type II diabetes mellitus), Glucocerebrosidase (Gaucher's disease), GM-CSF (regenerative effect, production of white blood cells, cancer), Heparin-binding EGF-like growth factor (HB-EGF) (wound healing, cardiac hypertrophy and heart development and function), Hepatocyte growth factor HGF (regenerative effect, wound healing), Hepcidin (iron metabolism disorders, Beta-thalassemia), Human albumin (Decreased production of albumin (hypoproteinaemia), increased loss of albumin (nephrotic syndrome), hypovolaemia, hyperbilirubinaemia), Idursulphase (Iduronate-2-sulphatase) (Mucopolysaccharidosis II (Hunter syndrome)), Integrins αVβ3, αVβ5 and α5β1 (Bind matrix macromolecules and proteinases, angiogenesis), Iuduronate sulfatase (Hunter syndrome), Laronidase (Hurler and Hurler-Scheie forms of mucopolysaccharidosis I), N-acetylgalactosamine-4-sulfatase (rhASB; galsulfase, Arylsulfatase A (ARSA), Arylsulfatase B (ARSB)) (arylsulfatase B deficiency, Maroteaux-Lamy syndrome, mucopolysaccharidosis VI), N-acetylglucosamine-6-sulfatase (Sanfilippo syndrome), Nerve growth factor (NGF, Brain-Derived Neurotrophic Factor (BDNF), Neurotrophin-3 (NT-3), and Neurotrophin 4/5 (NT-4/5) (regenerative effect, cardiovascular diseases, coronary atherosclerosis, obesity, type 2 diabetes, metabolic syndrome, acute coronary syndromes, dementia, depression, schizophrenia, autism, Rett syndrome, anorexia nervosa, bulimia nervosa, wound healing, skin ulcers, corneal ulcers, Alzheimer's disease), Neuregulin (NRG1, NRG2, NRG3, NRG4) (metabolic disorder, schizophrenia), Neuropilin (NRP-1, NRP-2) (angiogenesis, axon guidance, cell survival, migration), Obestatin (irritable bowel syndrome (IBS), obesity, Prader-Willi syndrome, type II diabetes mellitus), Platelet Derived Growth factor (PDGF (PDFF-A, PDGF-B, PDGF-C, PDGF-D) (regenerative effect, wound healing, disorder in angiogenesis, Arteriosclerosis, Fibrosis, cancer), TGF beta receptors (endoglin, TGF-beta 1 receptor, TGF-beta 2 receptor, TGF-beta 3 receptor) (renal fibrosis, kidney disease, diabetes, ultimately end-stage renal disease (ESRD), angiogenesis), Thrombopoietin (THPO) (Megakaryocyte growth and development factor (MGDF)) (platelets disorders, platelets for donation, recovery of platelet counts after myelosuppressive chemotherapy), Transforming Growth factor (TGF (TGF-alpha, TGF-beta (TGFbeta1, TGFbeta2, and TGFbeta3))) (regenerative effect, wound healing, immunity, cancer, heart disease, diabetes, Marfan syndrome, Loeys-Dietz syndrome), VEGF (VEGF-A, VEGF-B, VEGF-C, VEGF-D, VEGF-E, VEGF-F und PIGF) (regenerative effect, angiogenesis, wound healing, cancer, permeability), Nesiritide (Acute decompensated congestive heart failure), Trypsin (Decubitus ulcer, varicose ulcer, debridement of eschar, dehiscent wound, sunburn, meconium ileus), adrenocorticotrophic hormone (ACTH) (“Addison's disease, Small cell carcinoma, Adrenoleukodystrophy, Congenital adrenal hyperplasia, Cushing's syndrome, Nelson's syndrome, Infantile spasms), Atrial-natriuretic peptide (ANP) (endocrine disorders), Cholecystokinin (diverse), Gastrin (hypogastrinemia), Leptin (Diabetes, hypertriglyceridemia, obesity), Oxytocin (stimulate breastfeeding, non-progression of parturition), Somatostatin (symptomatic treatment of carcinoid syndrome, acute variceal bleeding, and acromegaly, polycystic diseases of the liver and kidney, acromegaly and symptoms caused by neuroendocrine tumors), Vasopressin (antidiuretic hormone) (diabetes insipidus), Calcitonin (Postmenopausal osteoporosis, Hypercalcaemia, Paget's disease, Bone metastases, Phantom limb pain, Spinal Stenosis), Exenatide (Type 2 diabetes resistant to treatment with metformin and a sulphonylurea), Growth hormone (GH), somatotropin (Growth failure due to GH deficiency or chronic renal insufficiency, Prader-Willi syndrome, Turner syndrome, AIDS wasting or cachexia with antiviral therapy), Insulin (Diabetes mellitus, diabetic ketoacidosis, hyperkalaemia), Insulin-like growth factor 1 IGF-1 (Growth failure in children with GH gene deletion or severe primary IGF1 deficiency, neurodegenerative disease, cardiovascular diseases, heart failure), Mecasermin rinfabate, IGF-1 analog (Growth failure in children with GH gene deletion or severe primary IGF1 deficiency, neurodegenerative disease, cardiovascular diseases, heart failure), Mecasermin, IGF-1 analog (Growth failure in children with GH gene deletion or severe primary IGF1 deficiency, neurodegenerative disease, cardiovascular diseases, heart failure), Pegvisomant (Acromegaly), Pramlintide (Diabetes mellitus, in combination with insulin), Teriparatide (human parathyroid hormone residues 1-34) (Severe osteoporosis), Becaplermin (Debridement adjunct for diabetic ulcers), Dibotermin-alpha (Bone morphogenetic protein 2) (Spinal fusion surgery, bone injury repair), Histrelin acetate (gonadotropin releasing hormone; GnRH) (Precocious puberty), Octreotide (Acromegaly, symptomatic relief of VIP-secreting adenoma and metastatic carcinoid tumours), and Palifermin (keratinocyte growth factor; KGF) (Severe oral mucositis in patients undergoing chemotherapy, wound healing).

These and other proteins are understood to be therapeutic, as they are meant to treat the subject by replacing its defective endogenous production of a functional protein in sufficient amounts. Accordingly, such therapeutic proteins are typically mammalian, in particular human proteins.

For the treatment of blood disorders, diseases of the circulatory system, diseases of the respiratory system, cancer or tumour diseases, infectious diseases or immunedeficiencies following therapeutic proteins may be used: Alteplase (tissue plasminogen activator; tPA) (Pulmonary embolism, myocardial infarction, acute ischaemic stroke, occlusion of central venous access devices), Anistreplase (Thrombolysis), Antithrombin III (AT-III) (Hereditary AT-III deficiency, Thromboembolism), Bivalirudin (Reduce blood-clotting risk in coronary angioplasty and heparin-induced thrombocytopaenia), Darbepoetin-alpha (Treatment of anaemia in patients with chronic renal insufficiency and chronic renal failure (+/−dialysis)), Drotrecogin-alpha (activated protein C) (Severe sepsis with a high risk of death), Erythropoietin, Epoetin-alpha, erythropoetin, erthropoyetin (Anaemia of chronic disease, myleodysplasia, anaemia due to renal failure or chemotherapy, preoperative preparation), Factor IX (Haemophilia B), Factor VIIa (Haemorrhage in patients with haemophilia A or B and inhibitors to factor VIII or factor IX), Factor VIII (Haemophilia A), Lepirudin (Heparin-induced thrombocytopaenia), Protein C concentrate (Venous thrombosis, Purpura fulminans), Reteplase (deletion mutein of tPA) (Management of acute myocardial infarction, improvement of ventricular function), Streptokinase (Acute evolving transmural myocardial infarction, pulmonary embolism, deep vein thrombosis, arterial thrombosis or embolism, occlusion of arteriovenous cannula), Tenecteplase (Acute myocardial infarction), Urokinase (Pulmonary embolism), Angiostatin (Cancer), Anti-CD22 immunotoxin (Relapsed CD33+ acute myeloid leukaemia), Denileukin diftitox (Cutaneous T-cell lymphoma (CTCL)), Immunocyanin (bladder and prostate cancer), MPS (Metallopanstimulin) (Cancer), Aflibercept (Non-small cell lung cancer (NSCLC), metastatic colorectal cancer (mCRC), hormone-refractory metastatic prostate cancer, wet macular degeneration), Endostatin (Cancer, inflammatory diseases like rheumatoid arthritis as well as Crohn's disease, diabetic retinopathy, psoriasis, and endometriosis), Collagenase (Debridement of chronic dermal ulcers and severely burned areas, Dupuytren's contracture, Peyronie's disease), Human deoxy-ribonuclease I, dornase (Cystic fibrosis; decreases respiratory tract infections in selected patients with FVC greater than 40% of predicted), Hyaluronidase (Used as an adjuvant to increase the absorption and dispersion of injected drugs, particularly anaesthetics in ophthalmic surgery and certain imaging agents), Papain (Debridement of necrotic tissue or liquefication of slough in acute and chronic lesions, such as pressure ulcers, varicose and diabetic ulcers, burns, postoperative wounds, pilonidal cyst wounds, carbuncles, and other wounds), L-Asparaginase (Acute lymphocytic leukaemia, which requires exogenous asparagine for proliferation), Peg-asparaginase (Acute lymphocytic leukaemia, which requires exogenous asparagine for proliferation), Rasburicase (Paediatric patients with leukaemia, lymphoma, and solid tumours who are undergoing anticancer therapy that may cause tumour lysis syndrome), Human chorionic gonadotropin (HCG) (Assisted reproduction), Human follicle-stimulating hormone (FSH) (Assisted reproduction), Lutropin-alpha (Infertility with luteinizing hormone deficiency), Prolactin (Hypoprolactinemia, serum prolactin deficiency, ovarian dysfunction in women, anxiety, arteriogenic erectile dysfunction, premature ejaculation, oligozoospermia, asthenospermia, hypofunction of seminal vesicles, hypoandrogenism in men), alpha-1-Proteinase inhibitor (Congenital antitrypsin deficiency), Lactase (Gas, bloating, cramps and diarrhoea due to inability to digest lactose), Pancreatic enzymes (lipase, amylase, protease) (Cystic fibrosis, chronic pancreatitis, pancreatic insufficiency, post-Billroth II gastric bypass surgery, pancreatic duct obstruction, steatorrhoea, poor digestion, gas, bloating), Adenosine deaminase (pegademase bovine, PEG-ADA) (Severe combined immunodeficiency disease due to adenosine deaminase deficiency), Abatacept (Rheumatoid arthritis (especially when refractory to TNFalpha inhibition)), Alefacept (Plaque Psoriasis), Anakinra (Rheumatoid arthritis), Etanercept (Rheumatoid arthritis, polyarticular-course juvenile rheumatoid arthritis, psoriatic arthritis, ankylosing spondylitis, plaque psoriasis, ankylosing spondylitis), Interleukin-1 (IL-1) receptor antagonist, Anakinra (inflammation and cartilage degradation associated with rheumatoid arthritis), Thymulin (neurodegenerative diseases, rheumatism, anorexia nervosa), TNF-alpha antagonist (autoimmune disorders such as rheumatoid arthritis, ankylosing spondylitis, Crohn's disease, psoriasis, hidradenitis suppurativa, refractory asthma), Enfuvirtide (HIV-1 infection), and Thymosin al (Hepatitis B and C).

(in brackets is the particular disease for which the therapeutic protein is used in the treatment)

In a further aspect, the present invention provides a vector comprising
a. an open reading frame (ORF) and/or a cloning site, e.g. for insertion of an open reading frame or a sequence comprising an open reading frame; and
b. at least one 3′-untranslated region element (3′-UTR element) and/or at least one 5′-untranslated region element (5′-UTR element), wherein the at least one 3′-UTR element and/or the at least one 5′-UTR element prolongs and/or increases protein production from said artificial nucleic acid molecule and wherein the at least one 3′-UTR element and/or the at least one 5′-UTR element is derived from a stable mRNA.

In general, the vector according to the present invention may comprise an artificial nucleic acid molecule according to the present invention as described above. In particular, the preferred embodiments described above for an artificial nucleic acid molecule according to the present invention also apply for an artificial nucleic acid molecule according to the present invention, which is comprised by a vector according to the present invention. For example, in the inventive vector the at least one 3′-UTR element and/or the at least one 5′-UTR element and the ORF are as described above for the artificial nucleic acid molecule according to the present invention, including the preferred embodiments. For example, in the vector according to the present invention, the stable mRNA from which the at least one 3′-UTR element and/or the at least one 5′-UTR element is derived may be preferably characterized by an mRNA decay wherein the ratio of the amount of said mRNA at a second point in time to the amount of said mRNA at a first point in time is at least 0.5 (50%), at least 0.6 (60%), at least 0.7 (70%), at least 0.75 (75%), at least 0.8 (80%), at least 0.85 (85%), at least 0.9 (90%), or at least 0.95 (95%).

The cloning site may be any sequence that is suitable for introducing an open reading frame or a sequence comprising an open reading frame, such as one or more restriction sites. Thus, the vector comprising a cloning site is preferably suitable for inserting an open reading frame into the vector, preferably for inserting an open reading frame 3′ to the 5′-UTR element and/or 5′ to the 3′-UTR element. Preferably the cloning site or the ORF is located 3′ to the 5′-UTR element and/or 5′ to the 3′-UTR element, preferably in close proximity to the 3′-end of the 5′-UTR element and/or to the 5′-end of the 3′-UTR element. For example, the cloning site or the ORF may be directly connected to the 3′-end of the 5′-UTR element and/or to the 5′-end of the 3′-UTR element or they may be connected via a stretch of nucleotides, such as by a stretch of 2, 4, 6, 8, 10, 20 etc. nucleotides as described above for the artificial nucleic acid molecule according to the present invention.

Preferably, the vector according to the present invention is suitable for producing the artificial nucleic acid molecule according to the present invention, preferably for producing an artificial mRNA according to the present invention, for example, by optionally inserting an open reading frame or a sequence comprising an open reading frame into the vector and transcribing the vector. Thus, preferably, the vector comprises elements needed for transcription, such as a promoter, e.g. an RNA polymerase promoter. Preferably, the vector is suitable for transcription using eukaryotic, prokaryotic, viral or phage transcription systems, such as eukaryotic cells, prokaryotic cells, or eukaryotic, prokaryotic, viral or phage in vitro transcription systems. Thus, for example, the vector may comprise a promoter sequence, which is recognized by a polymerase, such as by an RNA polymerase, e.g. by a eukaryotic, prokaryotic, viral, or phage RNA polymerase. In a preferred embodiment, the vector comprises a phage RNA polymerase promoter such as an SP6, T3 or T7, preferably a T7 promoter. Preferably, the vector is suitable for in vitro transcription using a phage based in vitro transcription system, such as a T7 RNA polymerase based in vitro transcription system.

In another preferred embodiment, the vector may be used directly for expression of the encoded peptide or protein in cells or tissue. For this purpose, the vector comprises particular elements, which are necessary for expression in those cells/tissue e.g. particular promoter sequences, such as a CMV promoter.

The vector may further comprise a poly(A) sequence and/or a polyadenylation signal as described above for the artificial nucleic acid molecule according to the present invention.

The vector may be an RNA vector or a DNA vector. Preferably, the vector is a DNA vector. The vector may be any vector known to the skilled person, such as a viral vector or a plasmid vector. Preferably, the vector is a plasmid vector, preferably a DNA plasmid vector.

In a preferred embodiment, the vector according to the present invention comprises the artificial nucleic acid molecule according to the present invention.

Preferably, a DNA vector according to the invention comprises a nucleic acid sequence which has an identity of at least about 1, 2, 3, 4, 5, 10, 15, 20, 30 or 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the nucleic acid sequence of a 3′-UTR of a transcript of a gene, such as to the nucleic acid sequences according to SEQ ID NOs: 1 to 24 and SEQ ID NOs: 49 to 318.

Preferably, a DNA vector according to the invention comprises a nucleic acid sequence which has an identity of at least about 1, 2, 3, 4, 5, 10, 15, 20, 30 or 40%, preferably of at least about 50%, preferably of at least about 60%, preferably of at least about 70%, more preferably of at least about 80%, more preferably of at least about 90%, even more preferably of at least about 95%, even more preferably of at least about 99%, most preferably of 100% to the nucleic acid sequence of a 5′-UTR of a transcript of a gene, such as to the nucleic acid sequences according to SEQ ID NOs: 25 to 30 and SEQ ID NOs: 319 to 382.

Preferably, a DNA vector according to the present invention comprises a sequence selected from the group consisting of DNA sequences according to SEQ ID NOs. 1 to 30 or a sequence having an identity of at least about 40%, preferably of at least about 50%, preferably of at least about 60%, preferably of at least about 70%, more preferably of at least about 80%, more preferably of at least about 90%, even more preferably of at least about 95%; even more preferably of at least about 99% sequence identity to the DNA sequences according to SEQ ID NOs. 1 to 30 or a fragment thereof as described above, preferably a functional fragment thereof.

Preferably, an RNA vector according to the present invention comprises a sequence selected from the group consisting of the sequences according to RNA sequences corresponding to DNA sequences according to SEQ ID NOs: 1 to 30 or a sequence having an identity of at least about 40%, preferably of at least about 50%, preferably of at least about 60%, preferably of at least about 70%, more preferably of at least about 80%, more preferably of at least about 90%, even more preferably of at least about 95%; even more preferably of at least about 99% sequence identity to the RNA sequences corresponding to the DNA sequences according to SEQ ID NOs: 1 to 30 or to a fragment thereof, preferably a functional fragment thereof.

Preferably, the vector is a circular molecule. Preferably, the vector is a double-stranded molecule, such as a double-stranded DNA molecule. Such circular, preferably double stranded DNA molecule may be used conveniently as a storage form for the inventive artificial nucleic acid molecule. Furthermore, it may be used for transfection of cells, for example, cultured cells. Also it may be used for in vitro transcription for obtaining an artificial RNA molecule according to the invention.

Preferably, the vector, preferably the circular vector, is linearizable, for example, by restriction enzyme digestion. In a preferred embodiment, the vector comprises a cleavage site, such as a restriction site, preferably a unique cleavage site, located immediately 3′ to the ORF, or—if present—located immediately 3′ to the 3′-UTR element, or—if present—located 3′ to the poly(A) sequence or polyadenylation signal, or
if present—located 3′ to the poly(C) sequence, or—if present—located 3′ to the histone stem-loop. Thus, preferably, the product obtained by linearizing the vector terminates at the 3′end with the 3′-end of the ORF, or—if present—with the 3′-end of the 3′-UTR element, or—if present—with the 3′-end of the poly(A) sequence or polyadenylation signal, or—if present—with the 3′-end of the poly(C) sequence. In the embodiment, wherein the vector according to the present invention comprises the artificial nucleic acid molecule according to the present invention, a restriction site, preferably a unique restriction site, is preferably located immediately 3′ to the 3′-end of the artificial nucleic acid molecule.

In a further aspect, the present invention relates to a cell comprising the artificial nucleic acid molecule according to the present invention or the vector according to present invention. The cell may be any cell, such as a bacterial cell, insect cell, plant cell, vertebrate cell, e.g. a mammalian cell. Such cell may be, e.g., used for replication of the vector of the present invention, for example, in a bacterial cell. Furthermore, the cell may be used for transcribing the artificial nucleic acid molecule or the vector according to the present invention and/or translating the open reading frame of the artificial nucleic acid molecule or the vector according to the present invention. For example, the cell may be used for recombinant protein production.

The cells according to the present invention are, for example, obtainable by standard nucleic acid transfer methods, such as standard transfection, transduction or transformation methods. For example, the artificial nucleic acid molecule or the vector according to the present invention may be transferred into the cell by electroporation, lipofection, e.g. based on cationic lipids and/or liposomes, calcium phosphate precipitation, nanoparticle based transfection, virus based transfection, or based on cationic polymers, such as DEAE-dextran or polyethylenimine etc.

Preferably, the cell is a mammalian cell, such as a cell of human subject, a domestic animal, a laboratory animal, such as a mouse or rat cell. Preferably the cell is a human cell. The cell may be a cell of an established cell line, such as a CHO, BHK, 293T, COS-7, HELA, HEK, etc. or the cell may be a primary cell, such as a human dermal fibroblast (HDF) cell etc., preferably a cell isolated from an organism. In a preferred embodiment, the cell is an isolated cell of a mammalian subject, preferably of a human subject. For example, the cell may be an immune cell, such as a dendritic cell, a cancer or tumor cell, or any somatic cell etc., preferably of a mammalian subject, preferably of a human subject.

In a further aspect, the present invention provides a pharmaceutical composition comprising the artificial nucleic acid molecule according to the present invention, the vector according the present invention, or the cell according to the present invention. The pharmaceutical composition according to the invention may be used, e.g., as a vaccine, for example, for genetic vaccination. Thus, the ORF may, e.g., encode an antigen to be administered to a patient for vaccination. Thus, in a preferred embodiment, the pharmaceutical composition according to the present invention is a vaccine. Furthermore, the pharmaceutical composition according to the present invention may be used, e.g., for gene therapy.

Preferably, the pharmaceutical composition further comprises one or more pharmaceutically acceptable vehicles, diluents and/or excipients and/or one or more adjuvants. In the context of the present invention, a pharmaceutically acceptable vehicle typically includes a liquid or non-liquid basis for the inventive pharmaceutical composition. In one embodiment, the pharmaceutical composition is provided in liquid form. In this context, preferably, the vehicle is based on water, such as pyrogen-free water, isotonic saline or buffered (aqueous) solutions, e.g phosphate, citrate etc. buffered solutions. The buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of mammalian cells due to osmosis or other concentration effects. Reference media are e.g. liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

One or more compatible solid or liquid fillers or diluents or encapsulating compounds suitable for administration to a patient may be used as well for the inventive pharmaceutical composition. The term “compatible” as used herein preferably means that these components of the inventive pharmaceutical composition are capable of being mixed with the inventive artificial nucleic acid, vector or cells as defined herein in such a manner that no interaction occurs which would substantially reduce the pharmaceutical effectiveness of the inventive pharmaceutical composition under typical use conditions.

The pharmaceutical composition according to the present invention may optionally further comprise one or more additional pharmaceutically active components. A pharmaceutically active component in this context is a compound that exhibits a therapeutic effect to heal, ameliorate or prevent a particular indication or disease. Such compounds include, without implying any limitation, peptides or proteins, nucleic acids, (therapeutically active) low molecular weight organic or inorganic compounds (molecular weight less than 5000, preferably less than 1000), sugars, antigens or antibodies, therapeutic agents already known in the prior art, antigenic cells, antigenic cellular fragments, cellular fractions, cell wall components (e.g. polysaccharides), modified, attenuated or de-activated (e.g. chemically or by irradiation) pathogens (virus, bacteria etc.).

Furthermore, the inventive pharmaceutical composition may comprise a carrier for the artificial nucleic acid molecule or the vector. Such a carrier may be suitable for mediating dissolution in physiological acceptable liquids, transport and cellular uptake of the pharmaceutical active artificial nucleic acid molecule or the vector. Accordingly, such a carrier may be a component which may be suitable for depot and delivery of an artificial nucleic acid molecule or vector according to the invention. Such components may be, for example, cationic or polycationic carriers or compounds which may serve as transfection or complexation agent.

Particularly preferred transfection or complexation agents in this context are cationic or polycationic compounds, including protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones.

Furthermore, such cationic or polycationic compounds or carriers may be cationic or polycationic peptides or proteins, which preferably comprise or are additionally modified to comprise at least one —SH moiety. Preferably, a cationic or polycationic carrier is selected from cationic peptides having the following sum formula (I):

{(Arg)ₗ;(Lys)ₘ;(His)ₙ;(Orn)ₒ;(Xaa)ₓ}; formula (I)

wherein l+m+n+o+x=3-100, and l, m, n or o independently of each other is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90 and 91-100 provided that the overall content of Arg (Arginine), Lys (Lysine), His (Histidine) and Orn (Ornithine) represents at least 10% of all amino acids of the oligopeptide; and Xaa is any amino acid selected from native (=naturally occurring) or non-native amino acids except of Arg, Lys, His or Orn; and x is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90, provided, that the overall content of Xaa does not exceed 90% of all amino acids of the oligopeptide. Any of amino acids Arg, Lys, His, Orn and Xaa may be positioned at any place of the peptide. In this context cationic peptides or proteins in the range of 7-30 amino acids are particular preferred.

Further, the cationic or polycationic peptide or protein, when defined according to formula {(Arg)ₗ;(Lys)m;(His)ₙ;(Orn)ₒ;(Xaa)ₓ} (formula (I)) as shown above and which comprise or are additionally modified to comprise at least one —SH moeity, may be, without being restricted thereto, selected from subformula (Ia):

{(Arg)ₗ;(Lys)ₘ;(His)ₙ;(Orn)ₒ;(Xaa′)ₓ(Cys)_{y}} subformula (Ia)

wherein (Arg)ₗ;(Lys)ₘ;(His)ₙ;(Orn)ₒ; and x are as defined herein, Xaa′ is any amino acid selected from native (=naturally occurring) or non-native amino acids except of Arg, Lys, His, Orn or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80 and 81-90, provided that the overall content of Arg (Arginine), Lys (Lysine), His (Histidine) and Orn (Ornithine) represents at least 10% of all amino acids of the oligopeptide. Further, the cationic or polycationic peptide may be selected from subformula (Ib):

Cys₁{(Arg)ₗ;(Lys)ₘ;(His)ₙ;(Orn)ₒ;(Xaa)ₓ}Cys₂ subformula (Ib)

wherein empirical formula {(Arg)ₗ;(Lys)ₘ;(His)ₙ;(Orn)ₒ;(Xaa)ₓ} (formula (III)) is as defined herein and forms a core of an amino acid sequence according to (semiempirical) formula (III) and wherein Cys₁ and Cys₂ are Cysteines proximal to, or terminal to (Arg)ₗ;(Lys)ₘ;(His)ₙ;(Orn)ₒ;(Xaa)ₓ.

Further preferred cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]-trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified Amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g polyethyleneglycole); etc.

According to another embodiment, the pharmaceutical composition according to the invention may comprise an adjuvant in order to enhance the immunostimulatory properties of the pharmaceutical composition. In this context, an adjuvant may be understood as any compound, which is suitable to support administration and delivery of the components such as the artificial nucleic acid molecule or vector comprised in the pharmaceutical composition according to the invention. Furthermore, such an adjuvant may, without being bound thereto, initiate or increase an immune response of the innate immune system, i.e. a non-specific immune response. With other words, when administered, the pharmaceutical composition according to the invention typically initiates an adaptive immune response directed to the antigen encoded by the artificial nucleic acid molecule. Additionally, the pharmaceutical composition according to the invention may generate an (supportive) innate immune response due to addition of an adjuvant as defined herein to the pharmaceutical composition according to the invention.

Such an adjuvant may be selected from any adjuvant known to a skilled person and suitable for the present case, i.e. supporting the induction of an immune response in a mammal. Preferably, the adjuvant may be selected from the group consisting of, without being limited thereto, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAPTM (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(bl-4)-N-acetylmuramyl-L-alanyl-D-isoglutamine); imiquimod (1-(2-methypropyl)-1H-imidazo[4,5-c]quinoline-4-amine); ImmTherTM (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3. TM; liposomes; LOXORIBINE™ (7-allyl-8-oxoguanosine); LT oral adjuvant (E. coli labile enterotoxin-protoxin); microspheres and microparticles of any composition; MF59TM; (squalene-water emulsion); MONTANIDE ISA 51TM (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (D-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, AL); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

Suitable adjuvants may also be selected from cationic or polycationic compounds wherein the adjuvant is preferably prepared upon complexing the artificial nucleic acid molecule or the vector of the pharmaceutical composition with the cationic or polycationic compound. Association or complexing the artificial nucleic acid molecule or the vector of the pharmaceutical composition with cationic or polycationic compounds as defined herein preferably provides adjuvant properties and confers a stabilizing effect to the artificial nucleic acid molecule or the vector of the pharmaceutical composition. Particularly such preferred, such cationic or polycationic compounds are selected from cationic or polycationic peptides or proteins, including protamine, nucleoline, spermin or spermidine, or other cationic peptides or proteins, such as poly-L-lysine (PLL), poly-arginine, basic polypeptides, cell penetrating peptides (CPPs), including HIV-binding peptides, Tat, HIV-1 Tat (HIV), Tat-derived peptides, Penetratin, VP22 derived or analog peptides, HSV VP22 (Herpes simplex), MAP, KALA or protein transduction domains (PTDs, PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, protamine, spermine, spermidine, or histones. Further preferred cationic or polycationic compounds may include cationic polysaccharides, for example chitosan, polybrene, cationic polymers, e.g. polyethyleneimine (PEI), cationic lipids, e.g. DOTMA: □1-(2,3-sioleyloxy)propyl)D-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]-trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as Q-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified Amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, Chitosan, etc., silan backbone based polymers, such as PMOXA-PDMS copolymers, etc., Blockpolymers consisting of a combination of one or more cationic blocks (e.g. selected of a cationic polymer as mentioned above) and of one or more hydrophilic- or hydrophobic blocks (e.g polyethyleneglycole); etc.

Additionally, preferred cationic or polycationic proteins or peptides, which can be used as an adjuvant by complexing the artificial nucleic acid molecule or the vector, preferably an RNA, of the composition, may be selected from following proteins or peptides having the following total formula (I): (Arg)l;(Lys)m;(His)n;(Orn)o;(Xaa)x, wherein l+m+n+o+x=8-15, and 1, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide. Particularly preferred oligoarginines in this context are e.g. Arg7, Arg8, Arg9, Arg7, H3R9, R9H3, H3R9H3, YSSR9SSY, (RKH)4, Y(RKH)2R, etc.

The ratio of the artificial nucleic acid or the vector to the cationic or polycationic compound may be calculated on the basis of the nitrogen/phosphate ratio (N/P-ratio) of the entire nucleic acid complex. For example, 1 μg RNA typically contains about 3 nmol phosphate residues, provided the RNA exhibits a statistical distribution of bases. Additionally, 1 μg peptide typically contains about x nmol nitrogen residues, dependent on the molecular weight and the number of basic amino acids. When exemplarily calculated for (Arg)9 (molecular weight 1424 g/mol, 9 nitrogen atoms), 1 μg (Arg)9 contains about 700 pmol (Arg)9 and thus 700×9=6300 pmol basic amino acids=6.3 nmol nitrogen atoms. For a mass ratio of about 1:1 RNA/(Arg)9 an N/P ratio of about 2 can be calculated. When exemplarily calculated for protamine (molecular weight about 4250 g/mol, 21 nitrogen atoms, when protamine from salmon is used) with a mass ratio of about 2:1 with 2 μg RNA, 6 nmol phosphate are to be calculated for the RNA; 1 μg protamine contains about 235 pmol protamine molecules and thus 235×21=4935 pmol basic nitrogen atoms=4.9 nmol nitrogen atoms. For a mass ratio of about 2:1 RNA/protamine an N/P ratio of about 0.81 can be calculated. For a mass ratio of about 8:1 RNA/protamine an N/P ratio of about 0.2 can be calculated. In the context of the present invention, an N/P-ratio is preferably in the range of about 0.1-10, preferably in a range of about 0.3-4 and most preferably in a range of about 0.5-2 or 0.7-2 regarding the ratio of nucleic acid:peptide in the complex, and most preferably in the range of about 0.7-1.5.

Patent application WO2010/037539, the disclosure of which is incorporated herein by reference, describes an immunostimulatory composition and methods for the preparation of an immunostimulatory composition. Accordingly, in a preferred embodiment of the invention, the composition is obtained in two separate steps in order to obtain both, an efficient immunostimulatory effect and efficient translation of the artificial nucleic acid molecule according to the invention. Therein, a so called “adjuvant component” is prepared by complexing—in a first step—the artificial nucleic acid molecule or vector, preferably an RNA, of the adjuvant component with a cationic or polycationic compound in a specific ratio to form a stable complex. In this context, it is important, that no free cationic or polycationic compound or only a neglibly small amount remains in the adjuvant component after complexing the nucleic acid. Accordingly, the ratio of the nucleic acid and the cationic or polycationic compound in the adjuvant component is typically selected in a range that the nucleic acid is entirely complexed and no free cationic or polycationic compound or only a neclectably small amount remains in the composition. Preferably the ratio of the adjuvant component, i.e. the ratio of the nucleic acid to the cationic or polycationic compound is selected from a range of about 6:1 (w/w) to about 0,25:1 (w/w), more preferably from about 5:1 (w/w) to about 0,5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w).

According to a preferred embodiment, the artificial nucleic acid molecule or vector, preferably an RNA molecule, according to the invention is added in a second step to the complexed nucleic acid molecule, preferably an RNA, of the adjuvant component in order to form the (immunostimulatory) composition of the invention. Therein, the artificial acid molecule or vector, preferably an RNA, of the invention is added as free nucleic acid, i.e. nucleic acid, which is not complexed by other compounds. Prior to addition, the free artificial nucleic acid molecule or vector is not complexed and will preferably not undergo any detectable or significant complexation reaction upon the addition of the adjuvant component.

Suitable adjuvants may furthermore be selected from nucleic acids having the formula (II): GlXmGn, wherein: G is guanosine, uracil or an analogue of guanosine or uracil; X is guanosine, uracil, adenosine, thymidine, cytosine or an analogue of the above-mentioned nucleotides; 1 is an integer from 1 to 40, wherein when 1=1 G is guanosine or an analogue thereof, when 1>1 at least 50% of the nucleotides are guanosine or an analogue thereof; m is an integer and is at least 3; wherein when m=3× is uracil or an analogue thereof, when m>3 at least 3 successive uracils or analogues of uracil occur; n is an integer from 1 to 40, wherein when n=1 G is guanosine or an analogue thereof, when n>1 at least 50% of the nucleotides are guanosine or an analogue thereof.

Other suitable adjuvants may furthermore be selected from nucleic acids having the formula (III): ClXmCn, wherein: C is cytosine, uracil or an analogue of cytosine or uracil; X is guanosine, uracil, adenosine, thymidine, cytosine or an analogue of the above-mentioned nucleotides; 1 is an integer from 1 to 40, wherein when l=1 C is cytosine or an analogue thereof, when 1>1 at least 50% of the nucleotides are cytosine or an analogue thereof; m is an integer and is at least 3; wherein when m=3× is uracil or an analogue thereof, when m>3 at least 3 successive uracils or analogues of uracil occur; n is an integer from 1 to 40, wherein when n=1 C is cytosine or an analogue thereof, when n>1 at least 50% of the nucleotides are cytosine or an analogue thereof.

The pharmaceutical composition according to the present invention preferably comprises a “safe and effective amount” of the components of the pharmaceutical composition, particularly of the inventive artificial nucleic acid molecule, the vector and/or the cells as defined herein. As used herein, a “safe and effective amount” means an amount sufficient to significantly induce a positive modification of a disease or disorder as defined herein. At the same time, however, a “safe and effective amount” preferably avoids serious side-effects and permits a sensible relationship between advantage and risk. The determination of these limits typically lies within the scope of sensible medical judgment.

In a further aspect, the present invention provides the artificial nucleic acid molecule according to the present invention, the vector according to the present invention, the cell according to the present invention, or the pharmaceutical composition according to the present invention for use as a medicament, for example, as vaccine (in genetic vaccination) or in gene therapy.

The artificial nucleic acid molecule according to the present invention, the vector according to the present invention, the cell according to the present invention, or the pharmaceutical composition according to the present invention are particularly suitable for any medical application which makes use of the therapeutic action or effect of peptides, polypeptides or proteins, or where supplementation of a particular peptide or protein is needed. Thus, the present invention provides the artificial nucleic acid molecule according to the present invention, the vector according to the present invention, the cell according to the present invention, or the pharmaceutical composition according to the present invention for use in the treatment or prevention of diseases or disorders amenable to treatment by the therapeutic action or effect of peptides, polypeptides or proteins or amenable to treatment by supplementation of a particular peptide, polypeptide or protein. For example, the artificial nucleic acid molecule according to the present invention, the vector according to the present invention, the cell according to the present invention, or the pharmaceutical composition according to the present invention may be used for the treatment or prevention of genetic diseases, autoimmune diseases, cancerous or tumour-related diseases, infectious diseases, chronic diseases or the like, e.g., by genetic vaccination or gene therapy.

In particular, such therapeutic treatments which benefit from an increased and prolonged presence of therapeutic peptides, polypeptides or proteins in a subject to be treated are especially suitable as medical application in the context of the present invention, since the inventive 3′-UTR element provides for a stable and prolonged expression of the encoded peptide or protein of the inventive artificial nucleic acid molecule or vector and/or the inventive 5′-UTR element provides for an increased expression of the encoded peptide or protein of the inventive artificial nucleic acid molecule or vector. Thus, a particularly suitable medical application for the artificial nucleic acid molecule according to the present invention, the vector according to the present invention, the cell according to the present invention, or the pharmaceutical composition according to the present invention is vaccination. Thus, the present invention provides the artificial nucleic acid molecule according to the present invention, the vector according to the present invention, the cell according to the present invention, or the pharmaceutical composition according to the present invention for vaccination of a subject, preferably a mammalian subject, more preferably a human subject. Preferred vaccination treatments are vaccination against infectious diseases, such as bacterial, protozoal or viral infections, and anti-tumour-vaccination. Such vaccination treatments may be prophylactic or therapeutic.

Depending on the disease to be treated or prevented, the ORF may be selected. For example, the open reading frame may code for a protein that has to be supplied to a patient suffering from total lack or at least partial loss of function of a protein, such as a patient suffering from a genetic disease. Additionally the open reading frame may be chosen from an ORF coding for a peptide or protein which beneficially influences a disease or the condition of a subject. Furthermore, the open reading frame may code for a peptide or protein which effects down-regulation of a pathological overproduction of a natural peptide or protein or elimination of cells expressing pathologically a protein or peptide. Such lack, loss of function or overproduction may, e.g., occur in the context of tumour and neoplasia, autoimmune diseases, allergies, infections, chronic diseases or the like. Furthermore, the open reading frame may code for an antigen or immunogen, e.g. for an epitope of a pathogen or for a tumour antigen. Thus, in preferred embodiments, the artificial nucleic acid molecule or the vector according to the present invention comprises an ORF encoding an amino acid sequence comprising or consisting of an antigen or immunogen, e.g. an epitope of a pathogen or a tumour-associated antigen, a 3′-UTR element as described above and/or a 5′-UTR element as described above, and optional further components, such as a poly(A) sequence etc.

In the context of medical application, in particular, in the context of vaccination, it is preferred that the artificial nucleic acid molecule according to the present invention is RNA, preferably mRNA, since DNA harbours the risk of eliciting an anti-DNA immune response and tends to insert into genomic DNA. However, in some embodiments, for example, if a viral delivery vehicle, such as an adenoviral delivery vehicle is used for delivery of the artificial nucleic acid molecule or the vector according to the present invention, e.g., in the context of gene therapeutic treatments, it may be desirable that the artificial nucleic acid molecule or the vector is a DNA molecule.

The artificial nucleic acid molecule according to the present invention, the vector according to the present invention, the cell according to the present invention, or the pharmaceutical composition according to the present invention may be administered orally, parenterally, by inhalation spray, topically, rectally, nasally, buccally, vaginally, via an implanted reservoir or via jet injection. The term parenteral as used herein includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, and sublingual injection or infusion techniques. In a preferred embodiment, the artificial nucleic acid molecule according to the present invention, the vector according to the present invention, the cell according to the present invention, or the pharmaceutical composition according to the present invention is administered via needle-free injection (e.g. jet injection).

Preferably, the artificial nucleic acid molecule according to the present invention, the vector according to the present invention, the cell according to the present invention, or the pharmaceutical composition according to the present invention is administered parenterally, e.g. by parenteral injection, more preferably by subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, sublingual injection or via infusion techniques. Particularly preferred is intradermal and intramuscular injection. Sterile injectable forms of the inventive pharmaceutical composition may be aqueous or oleaginous suspension. These suspensions may be formulated according to techniques known in the art using suitable dispersing or wetting agents and suspending agents. Preferably, the solutions or suspensions are administered via needle-free injection (e.g. jet injection).

The artificial nucleic acid molecule according to the present invention, the vector according to the present invention, the cell according to the present invention, or the pharmaceutical composition according to the present invention may also be administered orally in any orally acceptable dosage form including, but not limited to, capsules, tablets, aqueous suspensions or solutions.

The artificial nucleic acid molecule according to the present invention, the vector according to the present invention, the cell according to the present invention, or the pharmaceutical composition according to the present invention may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the artificial nucleic acid molecule according to the present invention, the vector according to the present invention, the cell according to the present invention, or the pharmaceutical composition according to the present invention may be formulated in a suitable ointment suspended or dissolved in one or more carriers.

In one embodiment, the use as a medicament comprises the step of transfection of mammalian cells, preferably in vitro or ex vivo transfection of mammalian cells, more preferably in vitro transfection of isolated cells of a subject to be treated by the medicament. If the use comprises the in vitro transfection of isolated cells, the use as a medicament may further comprise the readministration of the transfected cells to the patient. The use of the inventive artificial nucleic acid molecules or the vector as a medicament may further comprise the step of selection of successfully transfected isolated cells. Thus, it may be beneficial if the vector further comprises a selection marker. Also, the use as a medicament may comprise in vitro transfection of isolated cells and purification of an expression-product, i.e. the encoded peptide or protein from these cells. This purified peptide or protein may subsequently be administered to a subject in need thereof.

The present invention also provides a method for treating or preventing a disease or disorder as described above comprising administering the artificial nucleic acid molecule according to the present invention, the vector according to the present invention, the cell according to the present invention, or the pharmaceutical composition according to the present invention to a subject in need thereof.

Furthermore, the present invention provides a method for treating or preventing a disease or disorder comprising transfection of a cell with an artificial nucleic acid molecule according to the present invention or with the vector according to the present invention. Said transfection may be performed in vitro, ex vivo or in vivo. In a preferred embodiment, transfection of a cell is performed in vitro and the transfected cell is administered to a subject in need thereof, preferably to a human patient. Preferably, the cell which is to be transfected in vitro is an isolated cell of the subject, preferably of the human patient. Thus, the present invention provides a method of treatment comprising the steps of isolating a cell from a subject, preferably from a human patient, transfecting the isolated cell with the artificial nucleic acid according to the present invention or the vector according to the present invention, and administering the transfected cell to the subject, preferably the human patient.

The method of treating or preventing a disorder according to the present invention is preferably a vaccination method or a gene therapy method as described above.

As described above, the inventive 3′-UTR element and/or the inventive 5′-UTR element are capable of prolonging and/or increasing the protein production from an mRNA. Thus, in a further aspect, the present invention relates to a method for increasing and/or prolonging protein production from an artificial nucleic acid molecule, preferably from an mRNA molecule or a vector, the method comprising the step of associating an open reading frame with a 3′-UTR element and/or a 5′-UTR element, wherein the 3′-UTR element and/or the 5′-UTR element prolongs and/or increases protein production from a resulting artificial nucleic acid molecule and wherein the at least one 3′-UTR element and/or the at least one 5′-UTR element is derived from a stable mRNA, to obtain an artificial nucleic acid molecule, preferably an mRNA molecule, according to the present invention as described above or a vector according to the present invention as described above.

Preferably, in the method for increasing and/or prolonging protein production from an artificial nucleic acid molecule, preferably from an mRNA molecule or a vector, according to the present invention the 3′-UTR element and/or the 5′-UTR element comprises or consists of a nucleic acid sequence which is derived from the 3′-UTR and/or the 5′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), LTA4H, SLC38A6, DECR1, PIGK, FAM175A, PHYH, TBC1D19, PIGB, ALG6, CRYZ, BRP44L, ACADSB, SUPT3H, TMEM14A, GRAMD1C, C11orf80, C9orf46, ANXA4, TBCK, IFI6, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5,NT5DC1, RAB7A, AGA, TPK1, MBNL3, HADHB, MCCC2, CAT, ANAPC4, PCCB, PHKB, ABCB7, PGCP, GPD2, TMEM38B, NFU1, OMA1, LOC128322/NUTF2, NUBPL, LANCL1, HHLA3, PIR, ACAA2, CTBS, GSTM4, ALG8, Atp5e, Gstm5, Uqcr11, Ifi27I2a, Anapc13, Atp5I, Tmsb10, Nenf, Ndufa7, Atp5k, 1110008P4Rik, Cox4i1, Cox6a1, Ndufs6, Sec61b, Romo1, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Myeov2, Ndufs5, 1810027010Rik, Atp5o, Shfml, Tspo, S100a6, Taldol, Bloc1s1, Hexa, Ndufb11, Map11c3a, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Ybx1, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI; preferably from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonylreductase 2), MP68 (RIKEN cDNA 2010107E04 gene), NDUFA4 (NADH dehydrogenase (ubiquinone) 1 alpha subcomplex 4), Ybx1 (Y-Box binding protein 1), Ndufb8 (NADH dehydrogenase (ubiquinone) 1 beta subcomplex 8), and CNTN1 (contactin 1).

The term “associating the artificial nucleic acid molecule or the vector with a 3′-UTR element and/or a 5′-UTR element” in the context of the present invention preferably means functionally associating or functionally combining the artificial nucleic acid molecule or the vector with the 3′-UTR element and/or with the 5′-UTR element. This means that the artificial nucleic acid molecule or the vector and the 3′-UTR element and/or the 5′-UTR element, preferably the 3′-UTR element and/or the 5′-UTR element as described above, are associated or coupled such that the function of the 3′-UTR element and/or of the 5′-UTR element, e.g., the RNA and/or protein production prolonging and/or increasing function, is exerted. Typically, this means that the 3′-UTR element and/or the 5′-UTR element is integrated into the artificial nucleic acid molecule or the vector, preferably the mRNA molecule, 3′ and/or 5′, respectively, to an open reading frame, preferably immediately 3′ to an open reading frame and/or immediately 5′ to an open reading frame, the 3′-UTR element preferably between the open reading frame and a poly(A) sequence or a polyadenylation signal. Preferably, the 3′-UTR element and/or the 5′-UTR element is integrated into the artificial nucleic acid molecule or the vector, preferably the mRNA, as 3′-UTR and/or as 5′-UTR respectively, i.e. such that the 3′-UTR element and/or the 5′-UTR element is the 3′-UTR and/or the 5′-UTR, respectively, of the artificial nucleic acid molecule or the vector, preferably the mRNA, i.e., such that the 5′-UTR ends immediately before the 5′-end of the ORF and the 3′-UTR extends from the 3′-side of the open reading frame to the 5′-side of a poly(A) sequence or a polyadenylation signal, optionally connected via a short linker, such as a sequence comprising or consisting of one or more restriction sites. Thus, preferably, the term “associating the artificial nucleic acid molecule or the vector with a 3′-UTR element and/or a 5′-UTR element” means functionally associating the 3′-UTR element and/or the 5′-UTR element with an open reading frame located within the artificial nucleic acid molecule or the vector, preferably within the mRNA molecule. The 3′-UTR and/or the 5′-UTR and the ORF are as described above for the artificial nucleic acid molecule according to the present invention, for example, preferably the ORF and the 3′-UTR are heterologous and/or the ORF and the 5′-UTR are heterologous, respectively, e.g. derived from different genes, as described above.

In a further aspect, the present invention provides the use of a 3′-UTR element and/or of a 5′-UTR element, preferably the 3′-UTR element as described above and/or the 5′-UTR element as described above, for increasing and/or prolonging protein production from an artificial nucleic acid molecule, preferably from an mRNA molecule or a vector, wherein the 3′-UTR element and/or the 5′-UTR element comprises or consists of a nucleic acid sequence which is derived from the 3′-UTR and/or the 5′-UTR of a transcript of a gene selected from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), LTA4H, SLC38A6, DECR1, PIGK, FAM175A, PHYH, TBC1D19, PIGB, ALG6, CRYZ, BRP44L, ACADSB, SUPT3H, TMEM14A, GRAMD1C, C11orf80, C9orf46, ANXA4, TBCK, IF16, C2orf34, ALDH6A1, AGTPBP1, CCDC53, LRRC28, CCDC109B, PUS10, CCDC104, CASP1, SNX14, SKAP2, NDUFB6, EFHA1, BCKDHB, BBS2, LMBRD1, ITGA6, HERC5, NT5DC1, RAB7A, AGA, TPK1, MBNL3, HADHB, MCCC2, CAT, ANAPC4, PCCB, PHKB, ABCB7, PGCP, GPD2, TMEM38B, NFU1, OMA1, LOC128322/NUTF2, NUBPL, LANCL1, HHLA3, PIR, ACAA2, CTBS, GSTM4, ALG8, Atp5e, Gstm5, Uqcr11, Ifi27I2a, Anapc13, Atp5I, Tmsb10, Nenf, Ndufa7, Atp5k, 1110008P14Rik, Cox4i1, Cox6a1, Ndufs6, Sec61b, Romo1, Snrpd2, Mgst3, Aldh2, Ssr4, Myl6, Prdx4, Ubl5, 1110001J03Rik, Ndufa13, Ndufa3, Gstp2, Tmem160, Ergic3, Pgcp, Slpi, Myeov2, Ndufs5, 1810027010Rik, Atp5o, Shfml, Tspo, S100a6, Taldol, Bloc1s1, Hexa, Ndufb11, Map11c3a, Gpx4, Mif, Cox6b1, RIKEN cDNA2900010J23 (Swi5), Sec61g, 2900010M23Rik, Anapc5, Mars2, Phpt1, Ndufb8, Pfdn5, Arpc3, Ndufb7, Atp5h, Mrp123, Uba52, Tomm6, Mtch1, Pcbd2, Ecm1, Hrsp12, Meer, Uqcrq, Gstm3, Lsm4, Park7, Usmg5, Cox8a, Ly6c1, Cox7b, Ppib, Bag1, S100a4, Bcap31, Tecr, Rabac1, Robld3, Sod1, Nedd8, Higd2a, Trappc6a, Ldhb, Nme2, Snrpg, Ndufa2, Serf1, Oaz1, Rps4x, Rps13, Ybx1, Sepp1, Gaa, ACTR10, PIGF, MGST3, SCP2, HPRT1, ACSF2, VPS13A, CTH, NXT2, MGST2, C11orf67, PCCA, GLMN, DHRS1, PON2, NME7, ETFDH, ALG13, DDX60, DYNC2LI1, VPS8, ITFG1, CDK5, C1orf112, IFT52, CLYBL, FAM114A2, NUDT7, AKD1, MAGED2, HRSP12, STX8, ACAT1, IFT74, KIFAP3, CAPN1, COX11, GLT8D4, HACL1, IFT88, NDUFB3, ANO10, ARL6, LPCAT3, ABCD3, COPG2, MIPEP, LEPR, C2orf76, ABCA6, LY96, CROT, ENPP5, SERPINB7, TCP11L2, IRAK1BP1, CDKL2, GHR, KIAA1107, RPS6KA6, CLGN, TMEM45A, TBC1D8B, ACP6, RP6-213H19.1, SNRPN, GLRB, HERC6, CFH, GALC, PDE1A, GSTM5, CADPS2, AASS, TRIM6-TRIM34 (readthrough transcript), SEPP1, PDE5A, SATB1, CCPG1, CNTN1, LMBRD2, TLR3, BCAT1, TOM1L1, SLC35A1, GLYATL2, STAT4, GULP1, EHHADH, NBEAL1, KIAA1598, HFE, KIAA1324L, and MANSCI; preferably from the group consisting of GNAS (guanine nucleotide binding protein, alpha stimulating complex locus), MORN2 (MORN repeat containing 2), GSTM1 (glutathione S-transferase, mu 1), NDUFA1 (NADH dehydrogenase (ubiquinone) 1 alpha subcomplex), CBR2 (carbonyl reductase 2), MP68 (RIKEN cDNA 2010107E04 gene), NDUFA4 (NADH dehydrogenase (ubiquinone) 1 alpha subcomplex 4), Ybx1 (Y-Box binding protein 1), Ndufb8 (NADH dehydrogenase (ubiquinone) 1 beta subcomplex 8), and CNTN1 (contactin 1).

The uses according to the present invention preferably comprise associating the artificial nucleic acid molecule, the vector, or the RNA with the 3′-UTR element as described above and/or with the 5′-UTR element as described above.

The compounds and ingredients of the inventive pharmaceutical composition may also be manufactured and traded separately of each other. Thus, the invention relates further to a kit or kit of parts comprising an artificial nucleic acid molecule according to the invention, a vector according to the invention, a cell according to the invention, and/or a pharmaceutical composition according to the invention. Preferably, such kit or kits of parts may, additionally, comprise instructions for use, cells for transfection, an adjuvant, a means for administration of the pharmaceutical composition, a pharmaceutically acceptable carrier and/or a pharmaceutically acceptable solution for dissolution or dilution of the artificial nucleic acid molecule, the vector, the cells or the pharmaceutical composition.

In a further aspect the present invention provides a method for identifying a 3′-untranslated region element (3′-UTR element) and/or a 5′-untranslated region element (5′-UTR element), which is derived from a stable mRNA, comprising the following steps:
a) Analyzing the stability of an mRNA comprising the following sub-steps:
   i. Determining the amount of said mRNA at a first point in time during a decay process of said mRNA,
   ii. Determining the amount of said mRNA at a second point in time during a decay process of said mRNA, and
   iii. Calculating the ratio of the amount of said mRNA determined in step (i) to the amount of said mRNA determined in step (ii);
b) Selecting a stable mRNA having a ratio calculated in sub-step (iii) of at least 0.5 (50%), at least 0.6 (60%), at least 0.7 (70%), at least 0.75 (75%), at least 0.8 (80%), at least 0.85 (85%), at least 0.9 (90%), or at least 0.95 (95%); and
c) Determining the nucleotide sequence of a 3′- and/or 5′-UTR element of said stable mRNA.

Thereby, the stability of the mRNA is preferably assessed under standard conditions, for example standard conditions (standard medium, incubation, etc.) for a certain cell line or cell type used.

In order to analyze the stability of an mRNA, the decay process of this mRNA is assessed by determining the amount or concentration of said mRNA at a first and at a second point in time during the decay process of said mRNA (cf. steps a) i. and a) ii.).

To determine the amount or concentration of mRNA during the RNA decay process in vivo or in vitro as defined above (i.e. in vitro referring in particular to (“living”) cells and/or tissue, including tissue of a living subject; cells include in particular cell lines, primary cells, cells in tissue or subjects, preferred are mammalian cells, e.g. human cells and mouse cells and particularly preferred are the human cell lines HeLa, and U-937 and the mouse cell lines NIH3T3, JAWSII and L929 are used; furthermore primary cells are particularly preferred, in particular preferred embodiments human dermal fibroblasts (HDF)), various methods may be used, which are known to the skilled person. Non-limiting examples of such methods include general inhibition of transcription, e.g. with a transcription inhibitor such as Actinomycin D, use of inducible promotors to specifically promote transient transcription, e.g. c-fos serum-inducible promotor system and Tet-off regulatory promotor system, and kinetic labelling techniques, e.g. pulse labelling.

For example, if transcriptional inhibitor-mediated transcriptional arrest is used in step a) to determine the amount or concentration of mRNA during the RNA decay process in vivo or in vitro as defined above, transcriptional inhibitors such as Actinomycin D (ActD), 5,6-dichloro-1-D-ribofuranosyl-benzimidazole (DRB) or -amanitin (α-Am) may be used. Hereby, to assess mRNA decay, the transcriptional inhibitors are usually added to the cells and, thereby the transcription is generally inhibited and RNA decay can be observed without interferences of ongoing transcription.

Alternatively, inducible promotors to specifically promote transient transcription may be used in step a), whereby the rationale is to provide a stimulus that activates transcription and leads to a burst of mRNA synthesis, then remove the stimulus to shut off transcription and monitor the decay of mRNA. Thereby, the inducible promoter enables a stringent control, so that induction and silencing of transcription is accomplished within a narrow window of time. In mammalian cells, the cfos promoter is known to be valuable for this purpose, because it can be induced in response to serum addition quickly and transiently, thereby providing a reliable and simple way of achieving a transient burst in transcription. The Tet-off promotor system offers another option that further broadens the application of a transcriptional pulsing approach to study mRNA turnover in mammalian cells.

However, in the present invention kinetic labelling techniques are preferred in step a) for determining the amount of mRNA during the RNA decay process in vivo or in vitro as defined above. In kinetic labelling RNA is usually labelled, whereby labels include in particular labelled nucleotides and labelled nucleosides and labelled uridine and labelled uracil are particularly preferred. Examples of preferred labels include 4-thiouridine (4sU), 2-thiouridine, 6-thioguanosine, 5-ethynyluridine (EU), 5-bromo-uridine (BrU), Biotin-16-Aminoallyluridine, 5-Aminoallyluridine, 5-Aminoallylcytidine, etc., whereby 4-Thiouridine (4sU), 5-Ethynyluridine (EU) or 5′-Bromo-Uridine (BrU) are more preferred. Particularly preferred is 4-thiouridine (4sU). 4-Thiouridine (4sU) is preferably used in a concentration of 100-500 μM. Moreover, also radioactively labelled nucleotides may be used, e.g. with Uridine-³H. Also combinations of the above mentioned labelled nucleotides may be used, whereby a combination of 4-thiouridine and 6-thioguanosine is particularly preferred.

In kinetic labelling, usually the emerging RNA is labelled, e.g. by incorporation of labelled uridine or uracil during transcription. After a while, the provision of label is stopped and RNA decay may then be observed by assessing specifically labelled RNA without generally inhibiting transcription.

For determining the amount of mRNA during the RNA decay process in step a), pulse labelling is preferred, and a pulse-chase methodology is particularly preferred. As used herein, the term “pulse labelling refers to a technique in which a label, e.g. the labels described above, is used for the measurement of the rates of synthesis and/or decay of compounds within living cells. Typically, cells are exposed to a small quantity of a label for a brief period, hence the term ‘pulse’. In the pulse-chase methodology, after pulse-labelling usually a much larger quantity of an unlabeled compound corresponding to the “pulse” (e.g. unlabelled uridine, if labelled uridine is used as pulse) is added following the required period of exposure to the label.

The effect of competition between the labelled and the unlabeled compound is to reduce to a negligible level the further uptake of the labelled compound, hence the term “chase”.

To determine the amount or concentration of mRNA usually the mRNA has to be isolated. Different techniques for RNA isolation are known to the skilled person, e.g. by Guanidinium thiocyanate-phenol-chloroform extraction or by silica-column based extraction. Also commercially available kits may be used, e.g. RNeasy Kit from Qiagen.

Furthermore, an extraction step may be required, in particular if kinetic labelling is used (in contrast to a transcription inhibitor, wherein the total RNA represents “decaying” RNA since transcription is generally inhibited). In the extraction step, labelled RNA (i.e. representing “decaying” RNA) is extracted from total isolated RNA. Thus, the means of extraction may be selected depending on the label used. For example, immunopurification with antibodies to the label may be used.

Furthermore, for example, for extraction of thio-labelled, e.g. 4-thiouridine (4sU)-labelled, RNA, HPDP-Biotin (pyridyldithiol-activated, sulfhydryl-reactive biotinylation reagent that conjugates via a cleavable (reversible) disulfide bond) may be incubated with the isolated “total RNA”. This reagent specifically reacts with the reduced thiols (—SH) in the 4-thiouridine (4sU)-labelled RNA to form reversible disulfide bonds. The biotinylation allows for binding of the thio-labelled e.g. 4-thiouridine (4sU)-labelled RNA to streptavidin and therefore can be extracted from the total RNA by reduction of the disulfide bond with dithiothreitol or beta-mercaptoethanol (or any other reduction agent).

In case biotin-labelled nucleotides, e.g. Biotin-16-Aminoallyluridine, streptavidin can directly be used to extract the labelled RNA from total RNA.

For example, for extraction of newly transcribed 5-ethynyluridine (EU)-labelled cellular RNAs from total RNA, biotinylation of EU in a copper-catalyzed cycloaddition reaction (often referred to as click chemistry) may be used, which is followed by purification by streptavidin affinity. This method is commercially available as the Click-iT Nascent RNA Capture Kit (Catalog no. C10365, Invitrogen). The manufacturer's instruction of this kit recommends that the pulse labeling time is 30 to 60 min for a 0.5 mM EU dose, or 1 to 24 h for a 0.1 or 0.2 mM EU dose.

For example, BrU-labeled RNA molecules may be extracted by immunopurification with an anti-Bromodeoxyuridine antibody (e.g. Clone. 2B1, Catalog no. MI-11-3, MBL), and Protein G Sepharose.

The amount or concentration of mRNA, i.e. the transcript level, may then be measured by various methods known to the person skilled in the art. Non-limiting examples for such methods include micro array analysis, Northern Blot analysis, quantitative PCR or by next generation sequencing (high throughput sequencing). Particularly preferred are micro array analysis and next generation sequencing. Moreover, whole-genome approaches/whole transcriptome approaches are particularly preferred, e.g. in micro array analysis whole genome micro array analysis, e.g. Affymetrix Human Gene 1.0 ST or 2.0 ST or Affymetrix Mouse Gene 1.0 ST or 2.0 ST or whole transcriptome analysis by next generation sequencing.

In substeps i. and ii. of step a), the amount of mRNA is determined at a first and at a second point in time during a decay process of the mRNA. Typically, this means that mRNA is in particular isolated at a first and at a second point in time during a decay process of the mRNA to determine the respective amounts. Therefore, “the first point in time” and “the second point in time” are in particular points in time during the RNA decay process, at which RNA is isolated to determine the RNA amount. In general, “the second point in time” is later in the RNA decay process than the “the first point in time”.

Preferably, the first point in time is selected such, that only mRNA undergoing a decay process is considered, i.e. emerging mRNA—e.g. in ongoing transcription—is avoided. For example, if kinetic labelling techniques, e.g. pulse labelling, are used, the first point in time is preferably selected such that the incorporation of the label into mRNA is completed, i.e. no ongoing incorporation of the label into mRNA occurs. Thus, if kinetic labelling is used, the first point in time may be at least 10 min, at least 20 min, at least 30 min, at least 40 min, at least 50 min, at least 60 min, at least 70 min, at least 80 min, or at least 90 min after the end of the experimental labelling procedure, e.g. after the end of the incubation of cells with the label.

For example, the first point in time may be preferably from 0 to 6 h after the stop of transcription (e.g. by a transcriptional inhibitor), stop of promotor induction in case of inducible promotors or after stop of pulse or label supply, e.g. after end of labelling. More preferably, the first point in time may be from 30 min to 5 h, even more preferably from 1 h to 4 h and particularly preferably about 3 h after the stop of transcription (e.g. by a transcriptional inhibitor), stop of promotor induction in case of inducible promotors or after stop of pulse or label supply, e.g. after end of labelling.

Preferably, the second point in time is selected as late as possible during the mRNA decay process. However, if a plurality of mRNA species is considered, the second point in time is preferably selected such that still a considerable amount of the plurality of mRNA species, preferably at least 10% of the mRNA species, is present in a detectable amount, i.e. in an amount higher than 0. Preferably, the second point in time is at least 5 h, at least 6 h, at least 7 h, at least 8 h, at least 9 h, at least 10 h, at least 11 h, at least 12 h, at least 13 h, at least 14 h, or at least 15 h after the stop of transcription (e.g. by a transcriptional inhibitor), stop of promotor induction in case of inducible promotors or after stop of pulse or label supply, e.g. after end of labelling.

For example, the second point in time may be preferably from 3 to 48 h after the stop of transcription (e.g. by a transcriptional inhibitor), stop of promotor induction in case of inducible promotors or after stop of pulse or label supply, e.g. after end of labelling. More preferably, the second point in time may be from 6 min to 36 h, even more preferably from 10 h to 24 h and particularly preferably about 15 h after the stop of transcription (e.g. by a transcriptional inhibitor), stop of promotor induction in case of inducible promotors or after stop of pulse or label supply, e.g. after end of labelling.

Thus, the time span between the first point in time and the second point in time is preferably as large as possible within the above described limits. Therefore, the time span between the first point in time and the second point in time is preferably at least 4 h, at least 5 h, at least 6 h, at least 7 h, at least 8 h, at least 9 h, at least 10 h, at least 11 h, or at least 12 h, whereby a time span of about 12 h is particularly preferred. In general, the second later point in time is at least 10 minutes later than the first point in time.

In sub-step iii. of step a) the ratio of the amount of the mRNA determined in step (i) to the amount of the mRNA determined in step (ii) is calculated. To this end, the amount of the mRNA (transcript level) determined as described above at the second point in time is divided by the amount of the mRNA (transcript level) determined as described above at the first point in time. This ratio prevents that stable mRNAs, which are already at the first point in time present only in very low amounts, are disregarded in respect to mRNAs, which are present in high amounts.

In step b), such an mRNA is selected, which has a ratio calculated in sub-step (iii) of step a) of at least 0.5 (50%), at least 0.6 (60%), at least 0.7 (70%), at least 0.75 (75%), at least 0.8 (80%), at least 0.85 (85%), at least 0.9 (90%), or at least 0.95 (95%). Such mRNA is in the present invention considered as a particular stable mRNA.

In step c), the nucleotide sequence of a 3′- and/or 5′-UTR element of said mRNA, i.e. the mRNA selected in step b), is determined. To this end, different methods known to the skilled person may be applied, e.g. sequencing or selection from a publicly available database, such as e.g. NCBI (National Center for Biotechnology Information). For example, the mRNA sequence of the mRNA selected in step b) may be searched in a database and the 3′- and/or 5′-UTR may then be extracted from the mRNA sequence present in the database.

In particular, in the above described method for identifying a 3′-untranslated region element (3′-UTR element) and/or a 5′-untranslated region element (5′-UTR element), which is derived from a stable mRNA, the term “mRNA” and/or “stable mRNA”, respectively, may also refer to an mRNA species as defined herein and/or to a stable mRNA species, respectively.

Furthermore, it is preferred in the present invention that a “stable mRNA” may have a slower mRNA decay compared to average mRNA decay, preferably assessed in vivo or in vitro as defined above. Thereby, “average mRNA decay” may be assessed by investigating mRNA decay of a plurality of mRNA species.

Accordingly, the present invention provides in a further aspect a method for identifying a 3′-untranslated region element (3′-UTR element) and/or a 5′-untranslated region element (5′-UTR element), which is derived from a stable mRNA, comprising the following steps:
a) Analyzing the stability of a plurality of mRNA species comprising the following sub-steps:
   i. Determining the amount of each mRNA species of said plurality of mRNA species at a first point in time during a decay process of said mRNA species,
   ii. Determining the amount of each mRNA species of said plurality of mRNA species at a second point in time during a decay process of said mRNA species, and
   iii. Calculating for each mRNA species of said plurality of mRNA species the ratio of the amount of said mRNA species determined in step (i) to the the amount of said mRNA species determined in step (ii);
b) Ranking of the mRNA species of the plurality of mRNA species according to the ratio calculated in sub-step (iii) for each mRNA species;
c) Selecting one or more mRNA species having the highest ratio or the highest ratios calculated in sub-step (iii); and
d) Determining the nucleotide sequence of a 3′- and/or 5′-UTR element of said mRNA.

An “mRNA species”, as used herein, corresponds to a genomic transcription unit, i.e. usually to a gene. Thus, within one “mRNA species” different transcripts may occur, for example, due to mRNA processing. For example, an mRNA species may be represented by a spot on a microarray. Accordingly, a microarray provides an advantageous tool to determine the amount of a plurality of mRNA species, e.g. at a certain point in time during mRNA decay. However, also other techniques known to the skilled person, e.g. RNA-seq (also called Whole Transcriptome Shotgun Sequencing which is a technology that uses the capabilities of next-generation sequencing to reveal a snapshot of RNA presence and quantity from a genome at a given moment in time), quantitative PCR etc. may be used.

Preferably, “a plurality of mRNA species”, refers to at least 100, at least 300, at least 500, at least 1000, at least 2000, at least 3000, at least 4000, at least 5000, at least 6000, at least 7000, at least 8000, at least 9000, at least 10000, at least 11000, at least 12000, at least 13000, at least 14000, at least 15000, at least 16000, at least 17000, at least 18000, at least 19000, at least 20000, at least 21000, at least 22000, at least 23000, at least 24000, at least 25000, at least 26000, at least 27000, at least 28000, at least 29000, or at least 30000 mRNA species. It is particularly preferred that the whole transcriptome is assessed, or as many mRNA species of the transcriptome as possible. This may be achieved, for example, by using a micro array providing whole transcript coverage.

Step a) of this method with its sub-steps i. to iii. corresponds essentially to step a) with its sub-steps i. to iii. of the previously described inventive method, but differs only in that the amount of each mRNA species of a plurality of mRNA species is determined at a first and at a second point in time and in that the ratio is calculated for each mRNA species. Accordingly, the detailed methods and preferred embodiments outlined above apply here as well and the ratio for a single mRNA species (and each single mRNA species, respectively) may be determined as outlined above for “an mRNA”.

However, in contrast to the above method, the stability of the mRNA is not assessed by the absolute value of the ratio, but by a ranking of the mRNA species of the plurality of mRNA species according to the ratio calculated in sub-step (iii) of step a) for each mRNA species. In sub-step c) one or more mRNA species having the highest ratio or the highest ratios calculated in sub-step (iii) of step a) are then selected.

In this context it is particularly preferred to select the 0.1%, 0.2%, 0.3%, 0.4%, 0.5%, 0.6%, 0.7%, 0.8%, 0.9%, 1%, 2%, 3%, 4%, 5%, 10%, 15%, 20% most stable mRNA species in step c). Alternatively or additionally, in step c) such mRNA species may be selected which show a ratio calculated in sub-step iii. of step a) corresponding to a least 100% of the average ratio calculated from all mRNA species analyzed. More preferably such mRNA species are selected showing a ratio of at least 150%, even more preferably of at least 200% and most preferably of at least 300% of the average ratio calculated from all mRNA species analyzed.

In step d) the nucleotide sequence of a 3′- and/or 5′-UTR element of the mRNA selected in step c) is determined as described above, for step c) of the previously described inventive method.

Preferably, in both of the above described methods for identifying a 3′-UTR element and/or a 5′-UTR element according to the present invention, the time period between the first point in time and the second point in time is at least 5 h, preferably at least 6 h, preferably at least 7 h, more preferably at least 8 h, more preferably at least 9 h, even more preferably at least 10 h, even more preferably at least 11 h, and particularly preferably at least 12 h.

Preferably, in both of the above described methods for identifying a 3′-UTR element and/or a 5′-UTR element according to the present invention, the stability of an mRNA is analysed by pulse labelling, preferably using a pulse-chase methodology.

In a further aspect, the present invention also provides a method for identifying a 3′-untranslated region element (3′-UTR element) and/or a 5′-untranslated region element (5′-UTR element), which prolongs and/or increases protein production from an artificial nucleic acid molecule and which is derived from a stable mRNA comprising the following steps:
a) identifying a 3′-UTR element and/or a 5′-UTR element which is derived from a stable mRNA by a method for identifying a 3′-UTR element and/or a 5′-UTR element according to any of the methods described above;
b) synthesizing an artificial nucleic acid molecule comprising at least one open reading frame and at least one 3′-UTR element and/or at least one 5′-UTR element which corresponds to or is comprised by the 3′-UTR element and/or the 5′-UTR element identified in step a);
c) analyzing the expression of the protein encoded by the at least one open reading frame (ORF) of the artificial nucleic acid molecule synthesized in step b);
d) analyzing the expression of a protein encoded by at least one open reading frame of a reference artificial nucleic acid molecule lacking a 3′-UTR element and/or a 5′-UTR element;
e) comparing the protein expression from the artificial nucleic acid molecule analysed in step c) to the protein expression from the reference artificial nucleic acid molecule analysed in step d); and
f) selecting the 3′-UTR element and/or the 5′-UTR element if the protein expression from the artificial nucleic acid molecule analysed in step c) is prolonged and/or increased in comparison to the protein expression from the reference artificial nucleic acid molecule analysed in step d).

In this method, at first a 3′-UTR element and/or a 5′-UTR element are identified by a method according to the present invention as described above. This enables synthesis of the 3′- and/or the 5′-UTR element by methods known to the skilled person, e.g. by PCR amplification. The primers used for such a PCR may preferably comprise restriction sites for cloning. Alternatively, the 3′- and/or 5′-UTR element may be synthesized e.g. by chemical synthesis or oligo annealing. Accordingly, in step b), an artificial nucleic acid molecule is synthesized comprising at least one open reading frame and at least one 3′-UTR element and/or at least one 5′-UTR element which corresponds to or is comprised by the 3′-UTR element and/or the 5′-UTR element identified in step a). In particular, the at least one 3′-UTR element and/or at least one 5′-UTR element is usually combined with an open reading frame, which results in an artificial nucleic acid comprising a 3′- and/or 5′-UTR element according to the present invention, if the 3′- and/or 5′-UTR element fulfil the respective requirements, i.e. if they prolong and/or increase protein expression. To test this, the 3′- and/or the 5′-UTR element identified in step a), or a PCR fragment or synthesized sequence thereof respectively, may be cloned into a particular vector, preferably in an expression vector, in order to assess protein expression from the respective ORF.

The protein expression from the artificial nucleic acid molecule comprising the at least one 3′-UTR element and/or the at least one 5′-UTR element is then assessed in step c) as described herein and compared to the protein expression assessed in step d) from a respective reference artificial nucleic acid molecule lacking a 3′-UTR element and/or a 5′-UTR element as described herein in step e).

Thereafter, in step f), such a 3′-UTR element and/or 5′-UTR element is selected, which prolongs and/or increases the protein expression from the artificial nucleic acid molecule analysed in step c) in comparison to the protein expression from the reference artificial nucleic acid molecule analysed in step d). The comparison of the protein expression of the inventive nucleic acid molecule to the reference nucleic acid molecule is carried out as described herein, in particular in the context of the inventive artificial nucleic acid molecule.

Furthermore, the present invention provides a particularly preferred method for identifying a 3′-untranslated region element (3′-UTR element) and/or a 5′-untranslated region element (5′-UTR element), which prolongs and/or increases protein production from an artificial nucleic acid molecule and which is derived from a stable mRNA comprising the following steps:
a) feeding/incubating cells with a labelled nucleotide for incorporation in newly transcribed RNA molecules (pulse-chase labelling);
b) isolating total RNA of the cells at a first point in time and at at least one second later point in time;
c) extracting of the labelled RNA molecules from the total RNA isolated in step b);
d) measuring of the amount/transcript level of the different mRNA species comprised in the labelled RNA;
e) calculating the ratio of the amount/transcript level of an mRNA species present at the at least one second later point in time to the amount/transcript level of the mRNA species present at the first point in time;
f) ranking of the mRNA species according to the ratio determined in step e);
g) selecting the most stable mRNA species;
h) determinating the nucleotide sequence of the 3′- and/or 5′-UTR of the most stable mRNA species selected in step g);
i) synthesizing a 3′- and/or a 5′-UTR element comprised in the 3′- and/or 5′-UTR determined in step h);
j) combination of the 3′- and/or 5′-UTR element synthesized in step i) with an open reading frame to get a nucleic acid according to the invention as described herein; and
k) optionally comparing the expression of the open reading frame present in the inventive nucleic acid compared to the expression of the open reading frame present in a reference nucleic acid without a 3′- and/or 5′-UTR element as described herein.

Thereby, the details and preferred embodiments described for the inventive methods above also apply herein, within the respective limitation outlined in steps a) to k).

In particular, the following labelled nucleotides are preferred for feeding the cells in step a) of the inventive method: 4-thiouridine (4sU), 2-thiouridine, 6-thioguanosine, 5-ethynyluridine (EU), 5-bromo-uridine (BrU), Biotin-16-Aminoallyluridine, 5-Aminoallyluridine, 5-Aminoallylcytidine, etc. Particularly preferred is 4-thiouridine (4sU). 4-thiouridine is preferably used in a concentration of 100-500 μM.

Alternatively radioactively labelled nucleotides may be used, e.g. Uridine-³H. Combinations of the above mentioned labelled nucleotides may be used. Particularly preferred is the combination of 4-thiouridine and 6-thioguanosine

The incubation of the cells with the labelled nucleotide in step a) can be varied. Particularly preferred is an incubation (feeding time) from 10 minutes to 24 hours. Particularly preferred are 2 to 6 hours, more preferably 2 to 3 hours.

Cells, which can be used for the inventive method, include in particular cell lines, primary cells, cells in tissue or subjects. In specific embodiments cell types allowing cell culture may be suitable for the inventive method. Particularly preferred are mammalian cells, e.g. human cells and mouse cells. In particularly preferred embodiments the human cell lines HeLa, and U-937 and the mouse cell lines NIH3T3, JAWSII and L929 are used. Furthermore primary cells are particularly preferred; in particular preferred embodiments particularly human dermal fibroblasts (HDF) can be used. Alternatively the labelled nucleotide may also be applied to a tissue of a subject and after the incubation time the RNA of the tissue is isolated according to step c).

For determination of the most stable mRNAs of a cell (type), total RNA is extracted at a first point in time as described above, e.g. 0 to 6 h after labelling, preferably 3 h after labelling and at a second later point in time as described above, e.g. 3 to 48 h after labelling, preferably 10 to 24 h, most preferably 15 h after labelling. The second later point in time is at least 10 minutes later than the first time.

In step f) the mRNA species are ranked according to the ratio calculated in step e). In this context it is particularly preferred to select the 0.1%, 0.2%, 0.3%, 0.4%, 0.5%, 0.6%, 0.7%, 0.8%, 0.9%, 1%, 2%, 3%, 4%, 5%, 10%, 15%, 20% most stable mRNA species.

In this context it is further preferred to select these mRNA species showing at least 50% (0,5 fold), at least 60% (0,6 fold), at least 70% (0,7 fold), at least 90% (0,9 fold) or at least 95% (0,95 fold) transcript level/amount of the mRNA species at the second later time compared to the first time. This embodiment is particularly preferred if the RNA is isolated at 3 hours (first point in time) and at 15 hours (second point in time) after labelling.

Alternatively or additionally, these mRNA species are selected showing a ratio calculated in step e) corresponding to a least 100% of the average ratio calculated from all mRNA species analyzed. More preferably these mRNA species are selected showing a ratio of at least 150% and more preferably of at least 200% and most preferably of at least 300% of the average ratio calculated from all mRNA species analyzed.

In a further step of the inventive method the nucleotide sequence of the 3′- and/or 5′-UTR of the most stable mRNA species selected in step g) is determined and in step i) the 3′- and/or 5′-UTR element is synthesized e.g. by PCR amplification. The primers used for the PCR may preferably comprise restriction sites for cloning. Alternatively the 3′- and/or 5′-UTR element may be synthesized (e.g. by chemical synthesis or oligo annealing).

In step j) of the inventive method the resulting PCR fragment or synthesized sequence is combined with an open reading frame resulting in an artificial nucleic acid comprising a 3′- and/or 5′-UTR element according to the invention. Preferably, the PCR fragment or sequence may be cloned into a vector.

In a particularly preferred embodiment the invention provides a method comprising the steps a) to k) for identifying 3′-untranslated region elements (3′-UTR elements) and/or 5′-untranslated region elements (5′-UTR elements), wherein the 3′-UTR elements and/or the 5′-UTR elements prolong protein production from an artificial nucleic acid molecule comprising at least one of the 3′-UTR elements and/or at least one of the 5′-UTR elements.

In a further aspect, the present invention also provides a method for generating an artificial nucleic acid molecule, wherein an artificial nucleic acid molecule comprising at least one open reading frame and at least one 3′-UTR element and/or at least one 5′-UTR element identified by a method for identifying a 3′-UTR element and/or a 5′-UTR element according to the present invention as described above is synthesized. Synthesizing of such an artificial nucleic acid molecule is typically carried out by methods known to the skilled person, e.g. cloning methods for example as generally known or described herein.

Preferably, a vector according to the present invention as described herein is used in such an inventive method for generating an artificial nucleic acid molecule.

Preferably, the artificial nucleic acid molecule generated by such a method for generating an artificial nucleic acid molecule is a nucleic acid molecule according to the present invention as described herein.

In addition, the present invention also provides an artificial nucleic acid molecule obtainable by a method for generating an artificial nucleic acid molecule according to the present invention as described herein.

The following Figures, Sequences and Examples are intended to illustrate the invention further. They are not intended to limit the subject matter of the invention thereto.

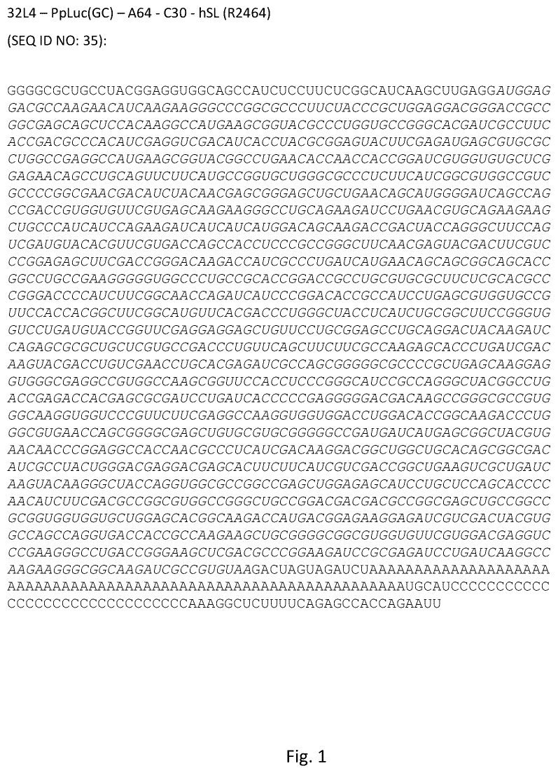

FIGS. 1 to 11, 19 to 21 and 25 to 30 show sequences encoding mRNAs that can be obtained by in vitro transcription. The following abbreviations are used:
PpLuc (GC): GC-enriched mRNA sequence coding for Photinus pyralis luciferase
A64: poly(A)-sequence with 64 adenylates
C30: poly(C)-sequence with 30 cytidylates
hSL: a histone stem-loop sequence taken from (Cakmakci, Lerner, Wagner, Zheng, & William F Marzluff, 2008. Mol. Cell. Biol. 28(3):1182-94)
32L4: 5′-UTR of human ribosomal protein Large 32 lacking the 5′ terminal oligopyrimidine tract
albumin7: 3′-UTR of human albumin with three single point mutations introduced to remove a T7 termination signal as well as a HindIII and XbaI restriction site
gnas: 3′-UTR element derived from the 3′-UTR of murine gnas; Mus musculus GNAS (guanine nucleotide binding protein, alpha stimulating) complex locus (Gnas), mRNA
morn2: 3′-UTR element derived from the 3′-UTR of murine morn2; Mus musculus MORN repeat containing 2 (Morn2), mRNA
gstm1: 3′-UTR element derived from the 3′-UTR of murine gstm1; Mus musculus glutathione S-transferase, mu 1 (Gstm1), mRNA
ndufa1: 3′-UTR element derived from the 3′-UTR of murine ndufa1; Mus musculus NADH dehydrogenase (ubiquinone) 1 alpha subcomplex, (Ndufa1), mRNA
cbr2: 3′-UTR element derived from the 3′-UTR of murine cbr2; Mus musculus carbonyl reductase 2 (Cbr2), mRNA
mp68: 5′-UTR element derived from the 5′-UTR of murine mp68; Mus musculus RIKEN cDNA 2010107E04 gene (2010107E04Rik), mRNA
ndufa4: 5′-UTR element derived from the 5′-UTR of murine nudfa4; Mus musculus NADH dehydrogenase (ubiquinone) 1 alpha subcomplex,4, (Ndufa4), mRNA
Ybx1: 3′-UTR element derived from the 3′-UTR of murine Ybx1 (Y-Box binding protein 1)
Ndufb8: 3′-UTR element derived from the 3′-UTR of murine Ndufb8 (NADH dehydrogenase (ubiquinone) 1 beta subcomplex 8)
CNTN1: 3′-UTR element derived from the 3′-UTR of human CNTN1 (contactin 1)

FIG. 1: shows SEQ ID NO. 35, i.e. the mRNA sequence of 32L4-PpLuc(GC)-A64-C30-hSL. (R2464). The 5′-UTR is derived of human ribosomal protein Large 32 mRNA lacking the 5′ terminal oligopyrimidine tract. The PpLuc(GC) ORF is highlighted in italics.

FIG. 2: shows SEQ ID NO. 36, i.e. the mRNA sequence of 32L4-PpLuc(GC)-gnas-A64-C30-hSL. (R3089). The PpLuc(GC) ORF is highlighted in italics. The 3′-UTR element, which is derived from mouse Gnas transcript, is underlined.

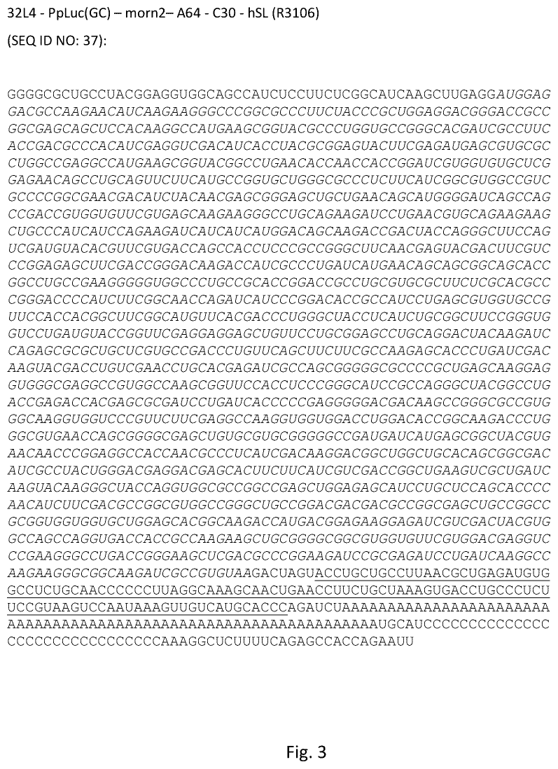

FIG. 3: shows SEQ ID NO. 37, i.e. the mRNA sequence of 32L4-PpLuc(GC)-morn2-A64-C30-hSL. (R3106). The PpLuc(GC) ORF is highlighted in italics. The 3′-UTR element, which is derived from mouse morn2, is underlined.

FIG. 4: shows SEQ ID NO. 38, i.e. the mRNA sequence of 32L4-PpLuc(GC)-gstm1-A64-C30-hSL. (R3107). The PpLuc(GC) ORF is highlighted in italics. The 3′-UTR element, which is derived from mouse gstm1, is underlined.

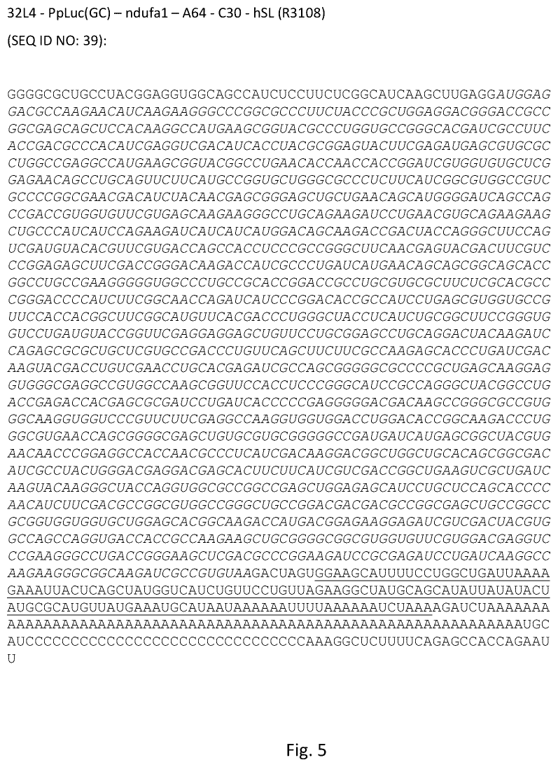

FIG. 5: shows SEQ ID NO. 39, i.e. the mRNA sequence of 32L4-PpLuc(GC)-ndufa1-A64-C30-hSL. (R3108). The PpLuc(GC) ORF is highlighted in italics. The 3′-UTR element, which is derived from mouse ndufa1, is underlined.

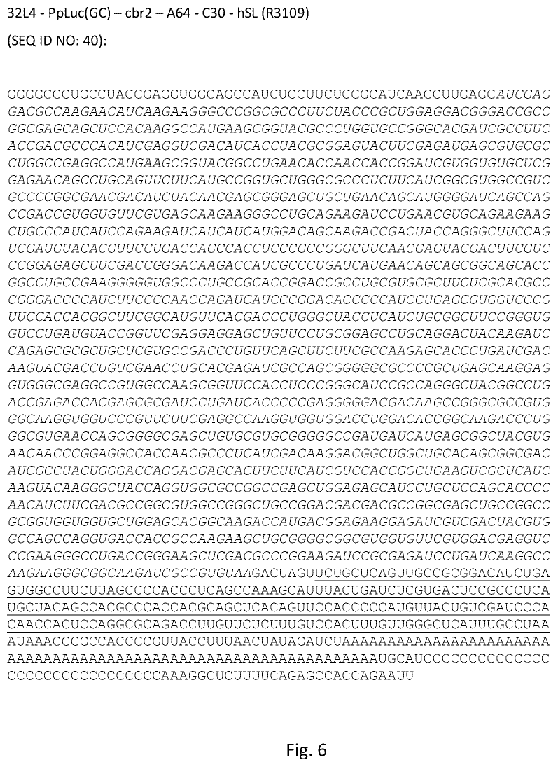

FIG. 6: shows SEQ ID NO. 40, i.e. the mRNA sequence of 32L4-PpLuc(GC)-cbr2-A64-C30-hSL. (R3109). The PpLuc(GC) ORF is highlighted in italics. The 3′-UTR element, which is derived from mouse cbr2, is underlined.

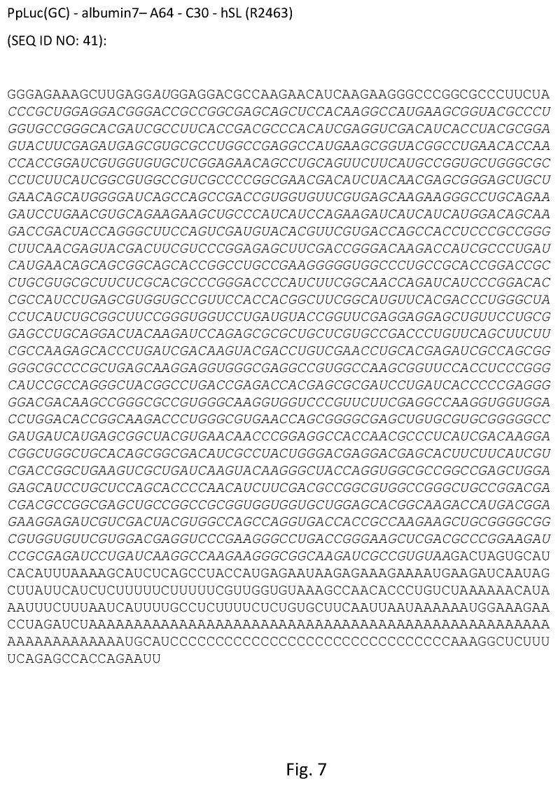

FIG. 7: shows SEQ ID NO. 41, i.e. the mRNA sequence of PpLuc(GC)-albumin7-A64-C30-hSL. (R2463). The 3′-UTR is derived from human albumin with three single point mutations introduced to remove a T7 termination signal as well as a HindIII and XbaI restriction site (albumin7). The PpLuc(GC) ORF is highlighted in italics.

FIG. 8: shows SEQ ID NO. 42, i.e. the mRNA sequence of Mp68-PpLuc(GC)-albumin7-A64-C30-hSL. (R3111). The PpLuc(GC) ORF is highlighted in italics. The 5′-UTR element, which is derived from mouse mp68, is underlined.

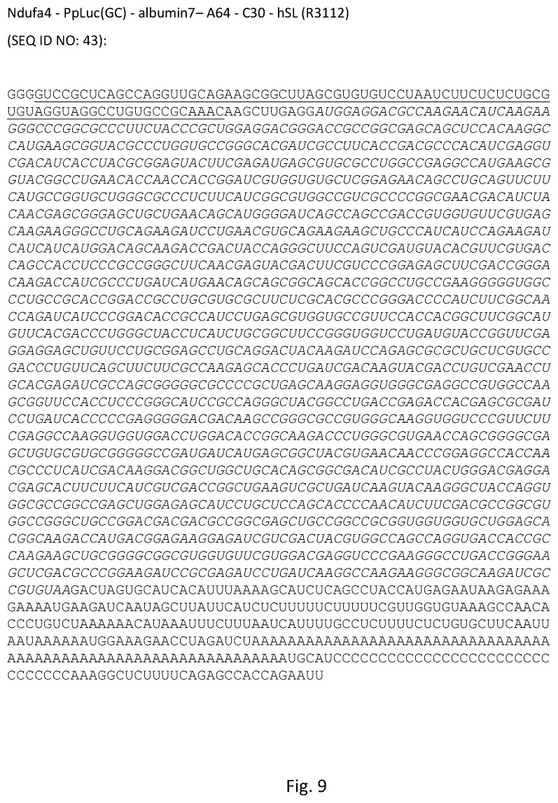

FIG. 9: shows SEQ ID NO. 43, i.e. the mRNA sequence of Ndufa4-PpLuc(GC)-albumin7-A64-C30-hSL. (R3112). The PpLuc(GC) ORF is highlighted in italics. The 5′-UTR element, which is derived from mouse Ndufa4, is underlined.

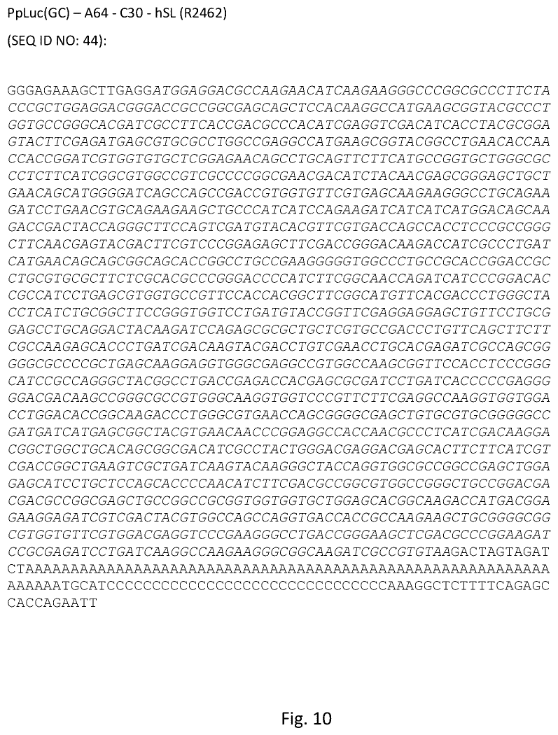

FIG. 10: shows SEQ ID NO. 44, i.e. the mRNA sequence of PpLuc(GC)-A64-C30-hSL (R2462) The PpLuc(GC) ORF is highlighted in italics.

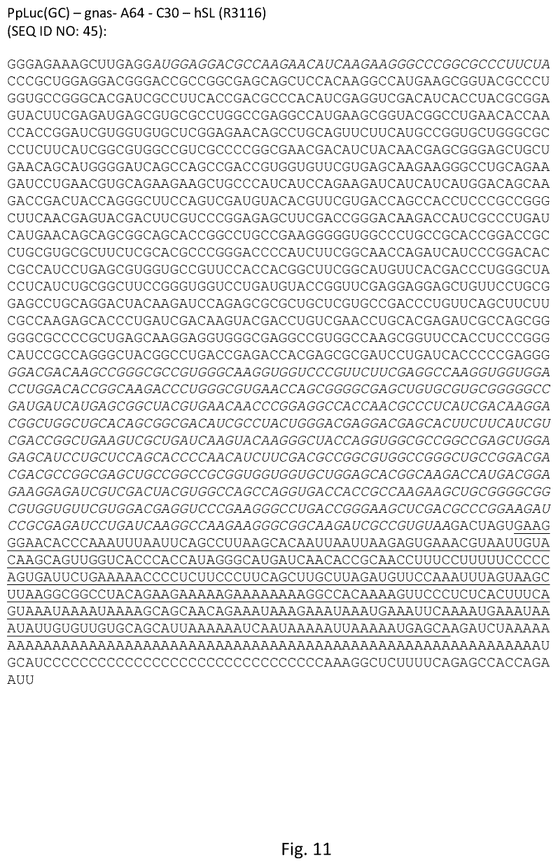

FIG. 11: shows SEQ ID NO. 45, i.e. the mRNA sequence of PpLuc(GC)-gnas-A64-C30-hSL (R3116). The PpLuc(GC) ORF is highlighted in italics. The 3′-UTR element, which is derived from mouse Gnas, is underlined.

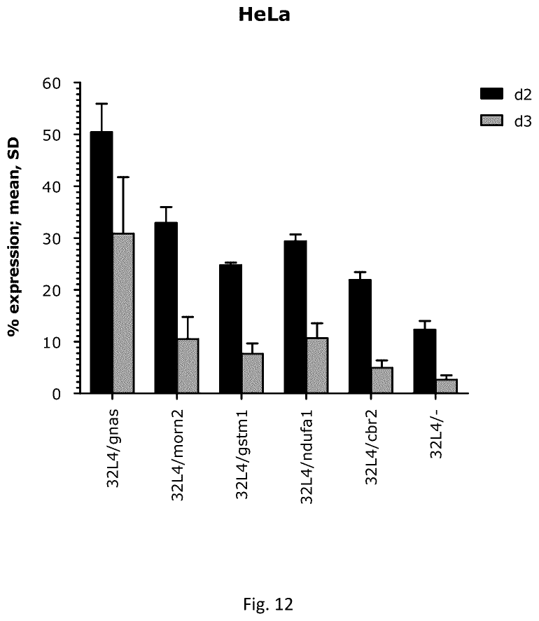

FIG. 12: shows that different 3′-UTR elements, namely 3′-UTR elements derived from gnas, morn2, gstm1, ndufa1 and cbr2 markedly prolong protein expression from mRNA. The effect of the inventive 3′-UTR elements derived from gnas, morn2, gstm1, ndufa1 and cbr2 3′-UTRs on luciferase expression from mRNA was examined, compared to luciferase expression from mRNA lacking a 3′-UTR. To this end, human HeLa were transfected with different mRNAs by lipofection. Luciferase levels were measured at different times after transfection. The PpLuc signal was corrected for transfection efficiency by the signal of cotransfected RrLuc. Normalized PpLuc levels at 24 h were set to 100% and relative expression to 24 h was calculated. The 3′-UTRs prolong luciferase expression. Mean values from three independent experiments are shown. Values are summarized in Example 7.a.

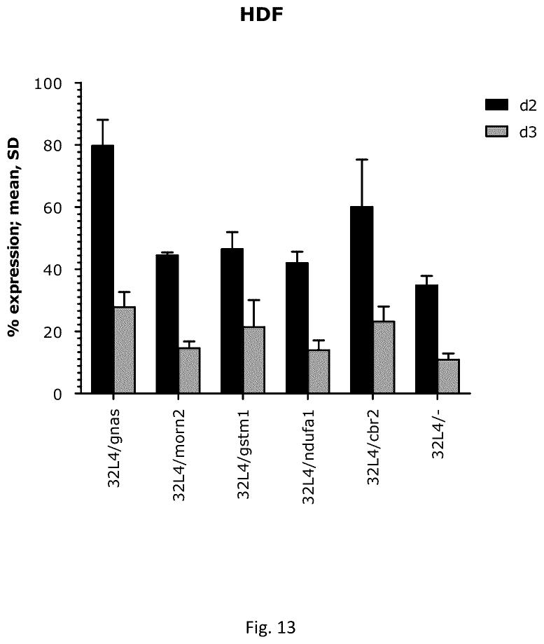

FIG. 13: shows that different 3′-UTR elements, namely 3′-UTR elements derived from gnas, morn2, gstm1, ndufa1 and cbr2 markedly prolong protein expression from mRNA.

The effect of the inventive 3′-UTR elements derived from gnas, morn2, gstm1, ndufa1 and cbr2 3′-UTRs on luciferase expression from mRNA was examined, compared to luciferase expression from mRNA lacking a 3′-UTR. To this end, HDF (human dermal fibroblasts) cells were transfected with different mRNAs by lipofection. Luciferase levels were measured at different times after transfection. The PpLuc signal was corrected for transfection efficiency by the signal of cotransfected RrLuc. Normalized PpLuc levels at 24 h were set to 100% and relative expression to 24 h was calculated. The 3′-UTRs prolong luciferase expression. Mean values from three independent experiments are shown. Values are summarized in Example 7.a.

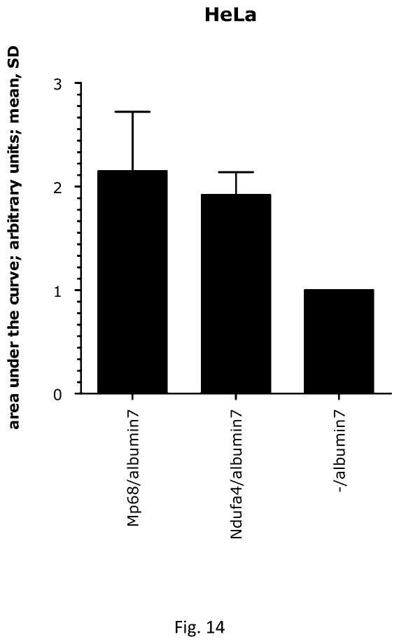

FIG. 14: shows that different 5′-UTR elements, namely 5′-UTR elements derived from Mp68 and ndufa4 markedly increase total protein expression from mRNA.

The effect of the inventive 5′-UTR elements derived from Mp68 and ndufa4 on luciferase expression from mRNA was examined. To this end, human HeLa cells were transfected with different mRNAs by lipofection. Luciferase levels were measured 6, 24, 48, and 72 hours after transfection. The PpLuc signal was corrected for transfection efficiency by the signal of cotransfected RrLuc. Total protein expression (area under the curve) was calculated. To compare the expression levels of the mRNAs containing the inventive 5′-UTR elements to an mRNA lacking a 5′-UTR, expression levels of the control construct without 5′ UTR was set to 1. Mean values from three independent experiments are shown. Values are summarized in Example 7.b.

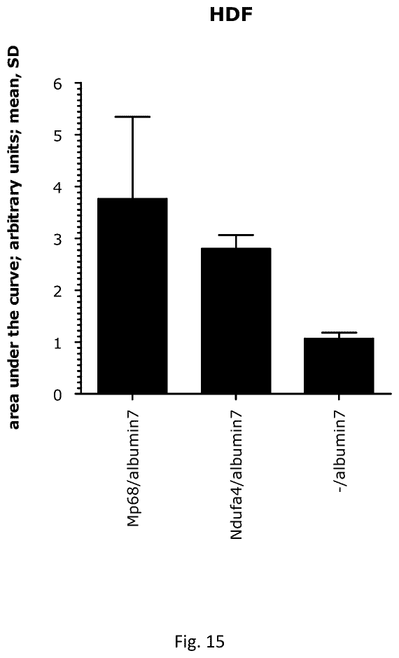

FIG. 15: shows that different 5′-UTR elements, namely 5′-UTR elements derived from Mp68 and ndufa4 markedly increase total protein expression from mRNA.

The effect of the inventive 5′-UTR elements derived from Mp68 and ndufa4 on luciferase expression from mRNA was examined. To this end, HDF cells were transfected with different mRNAs by lipofection. Luciferase levels were measured 6, 24, 48, and 72 hours after transfection. The PpLuc signal was corrected for transfection efficiency by the signal of cotransfected RrLuc. Total protein expression (area under the curve) was calculated. To compare the expression levels of the mRNAs containing the inventive 5′-UTR elements to an mRNA lacking a 5′-UTR, expression levels of the control construct without 5′ UTR was set to 1. Mean values from three independent experiments are shown. Values are summarized in Example 7.b.

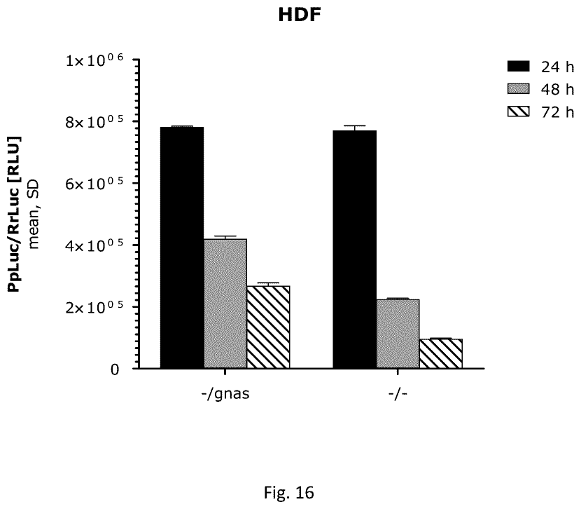

FIG. 16: shows that the 3′-UTR element derived from gnas markedly prolongs protein expression from mRNA.

The effect of the inventive 3′-UTR element derived from gnas 3′-UTR on luciferase expression from mRNA was examined, compared to luciferase expression from mRNA lacking a 3′-UTR. To this end, HDF cells were transfected with respective mRNAs by lipofection. Luciferase levels were measured at 24, 48, and 72 hours after transfection. The PpLuc signal was corrected for transfection efficiency by the signal of cotransfected RrLuc. Normalized PpLuc levels at 24 h were set to 100% and relative expression to 24 h was calculated. The gnas 3′-UTR prolongs luciferase expression. Values are summarized in Example 7.c.

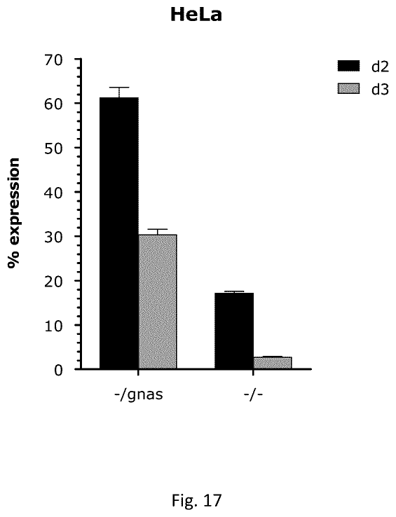

FIG. 17: shows that the 3′-UTR element derived from gnas markedly prolongs protein expression from mRNA.

The effect of the inventive 3′-UTR element derived from gnas 3′-UTR on luciferase expression from mRNA was examined, compared to luciferase expression from mRNA lacking a 3′-UTR. To this end, HeLa cells were transfected with respective mRNAs by lipofection. Luciferase levels were measured at d2 and d3 after transfection. The PpLuc signal was corrected for transfection efficiency by the signal of cotransfected RrLuc. Normalized PpLuc levels at 24 h were set to 100% and relative expression to 24 h was calculated. The gnas 3′-UTR prolongs luciferase expression. Values are summarized in Example 7.c.

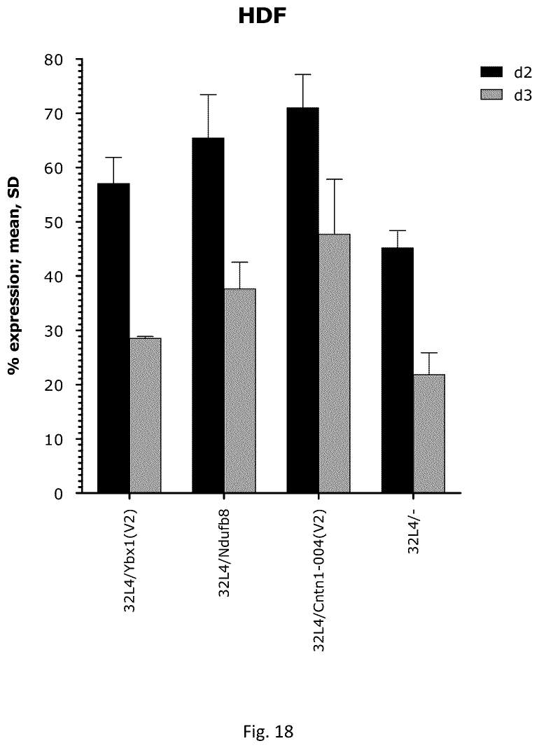

FIG. 18: shows that different 3′-UTR elements, namely 3′-UTR elements derived from ybx1(V2), ndufb8, and cntnl-004(V2) markedly prolong protein expression from mRNA.

The effect of the inventive 3′-UTR elements derived from ybx1(V2), ndufb8, and cntn1-004(V2) 3′-UTRs on luciferase expression from mRNA was examined, compared to luciferase expression from mRNA lacking a 3′-UTR. To this end, HDF cells were transfected with the different mRNAs by lipofection. Luciferase levels were measured at different times after transfection. The PpLuc signal was corrected for transfection efficiency by the signal of cotransfected RrLuc. Normalized PpLuc levels at 24 h were set to 100% and relative expression to 24 h was calculated. The 3′-UTRs prolong luciferase expression. Values are summarized in Example 7.d.

FIG. 19: shows SEQ ID NO. 46, i.e. the mRNA sequence of 32L4-PpLuc(GC)-Ybx1-001(V2)-A64-C30-hSL (R3623) Mus musculus 3′UTR with mutation T128bpG and deletion del236-237 bp.

The PpLuc(GC) ORF is highlighted in italics. The 3′-UTR element, which is derived from mouse Ybx1 transcript, is underlined.

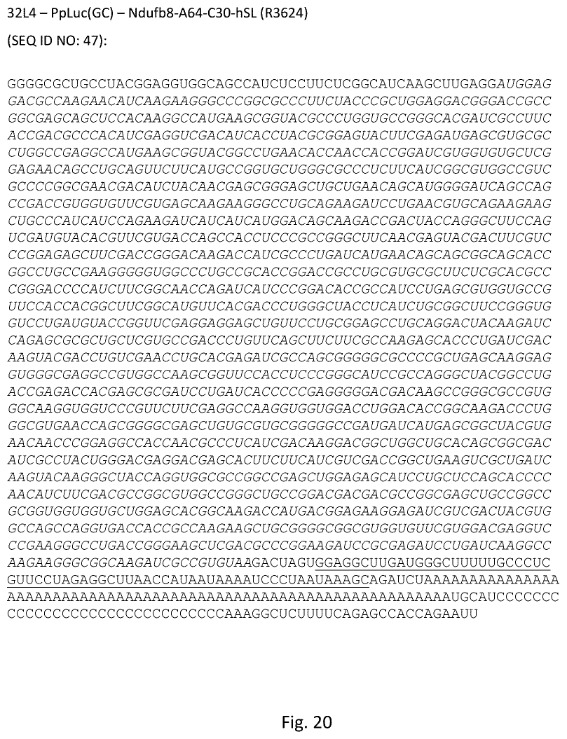

FIG. 20: shows SEQ ID NO. 47, i.e. the mRNA sequence of 32L4-PpLuc(GC)-Ndufb8-A64-C30-hSL (R3624) The PpLuc(GC) ORF is highlighted in italics. The 3′-UTR element, which is derived from mouse Ndufb8 transcript, is underlined.

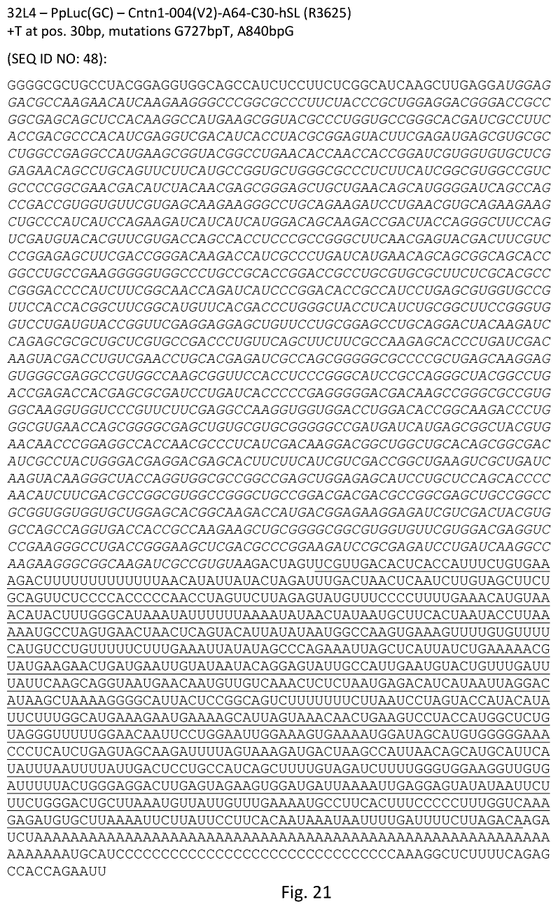

FIG. 21: shows SEQ ID NO. 48, i.e. the mRNA sequence of 32L4-PpLuc(GC)-Cntn1-004(V2)-A64-C30-hSL (R3625)+T at pos. 30 bp, mutations G727bpT, A840bpG. The PpLuc(GC) ORF is highlighted in italics. The 3′-UTR element, which is derived from human Cntn1 transcript, is underlined.

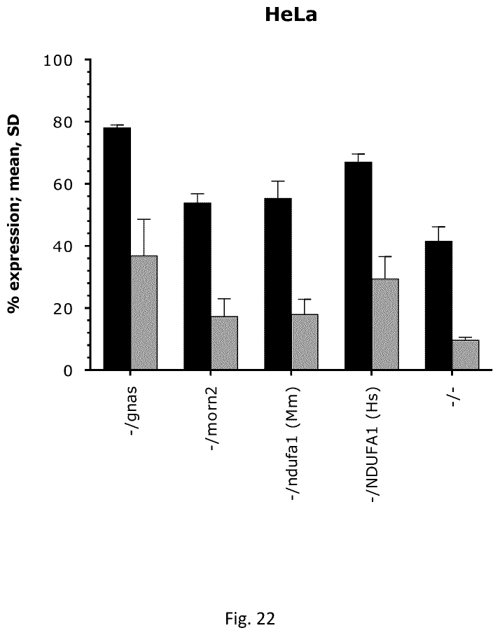

FIG. 22: shows that different 3′-UTR elements, namely 3′-UTR elements derived from gnas, morn2, ndufa1 (Mm; Mus musculus), and NDUFA1 (Hs; Homo sapiens) markedly prolong protein expression from mRNA. The effect of the inventive 3′-UTR elements derived from gnas, morn2, ndufa1 (Mm; Mus musculus), and NDUFA1 (Hs; Homo sapiens) on luciferase expression from mRNA was examined, compared to luciferase expression from mRNA lacking a 3′-UTR. To this end, human Hela cells were transfected with respective mRNAs by lipofection. Luciferase levels were measured at different times after transfection. The PpLuc signal was corrected for transfection efficiency by the signal of cotransfected RrLuc. Normalized PpLuc levels at 24 h were set to 100% and relative expression to 24 h is calculated. The 3′UTRs prolong luciferase expression. Mean values from 3 independent experiments are shown. Values are summarized in Table 8.

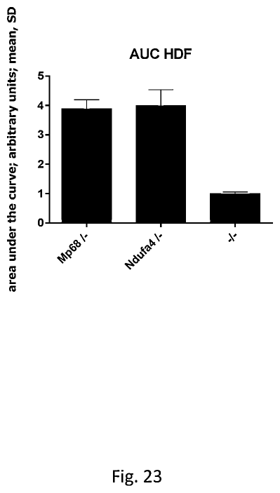

FIG. 23: shows that different 5′-UTR elements, namely 5′-UTR elements derived from Mp68 and ndufa4, markedly increase total protein expression from mRNA. The effect of the inventive 5′-UTR elements derived from Mp68 and ndufa4 on luciferase expression from mRNA was examined. To this end, human HeLa cells were transfected with different mRNAs by lipofection. Luciferase levels were measured 6, 24, 48, and 72 hours after transfection. The PpLuc signal was corrected for transfection efficiency by the signal of cotransfected RrLuc. Total protein expression (area under the curve) was calculated. To compare the expression levels of the mRNAs containing the inventive 5′-UTR elements to an mRNA lacking a 5′-UTR, expression levels of the control construct without 5′ UTR was set to 1. Mean values are shown. Values are summarized in Table 9.

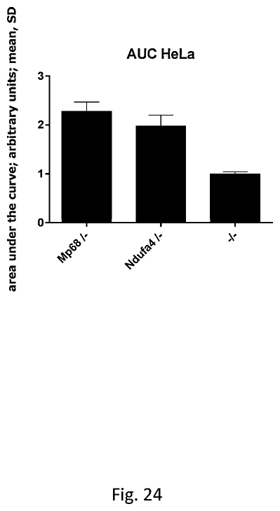

FIG. 24: shows that different 5′-UTR elements, namely 5′-UTR elements derived from Mp68 and ndufa4, markedly increase total protein expression from mRNA. The effect of the inventive 5′-UTR elements derived from Mp68 and ndufa4 on luciferase expression from mRNA was examined. To this end, human Hela cells were transfected with different mRNAs by lipofection. Luciferase levels were measured 24, 48, and 72 hours after transfection. The PpLuc signal was corrected for transfection efficiency by the signal of cotransfected RrLuc. Total protein expression (area under the curve) was calculated. To compare the expression levels of the mRNAs containing the inventive 5′-UTR elements to an mRNA lacking a 5′-UTR, expression levels of the control construct without 5′ UTR was set to 1. Mean values are shown. Values are summarized in Table 9.

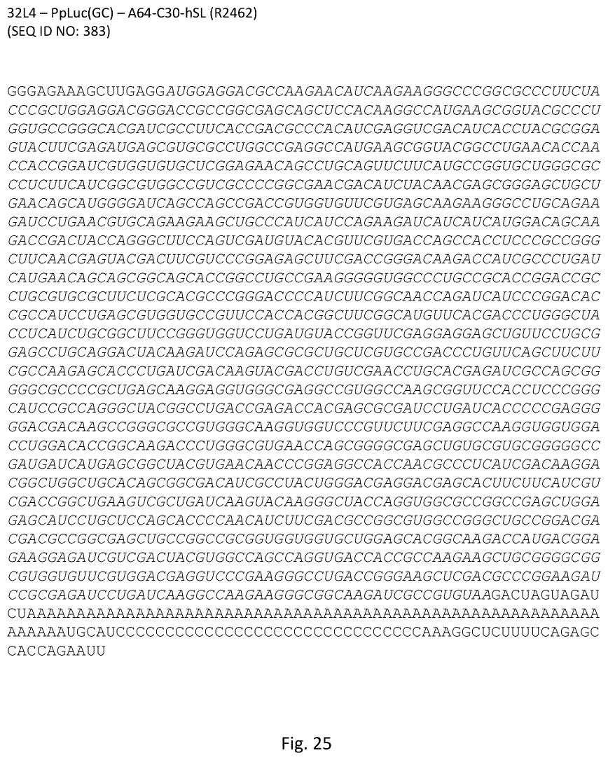

FIG. 25: shows SEQ ID NO. 383, i.e. the mRNA sequence of 32L4-PpLuc(GC)-A64-C30-hSL (R2462). The PpLuc(GC) ORF is highlighted in italics.

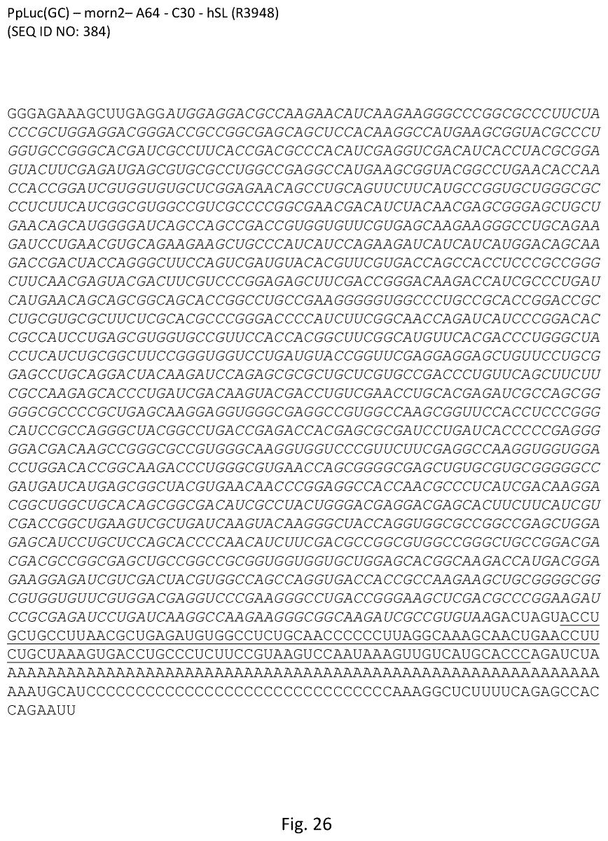

FIG. 26: shows SEQ ID NO. 384, i.e. the mRNA sequence of PpLuc(GC)-morn2-A64-C30-hSL. (R3948). The PpLuc(GC) ORF is highlighted in italics. The 3′-UTR element, which is derived from murine morn2 is underlined.

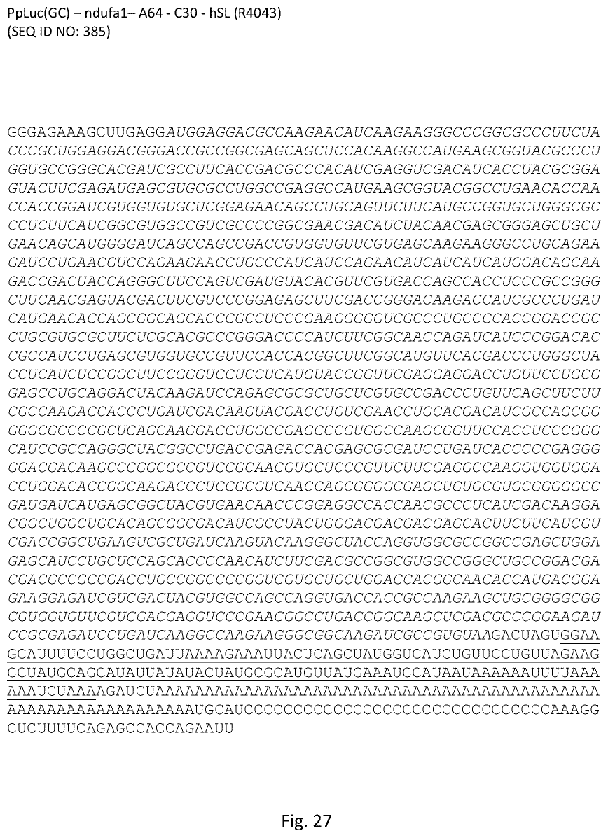

FIG. 27: shows SEQ ID NO. 385, i.e. the mRNA sequence of PpLuc(GC)-ndufa1-A64-C30-hSL. (R4043). The PpLuc(GC) ORF is highlighted in italics. The 3′-UTR element, which is derived from murine ndufa1 is underlined.

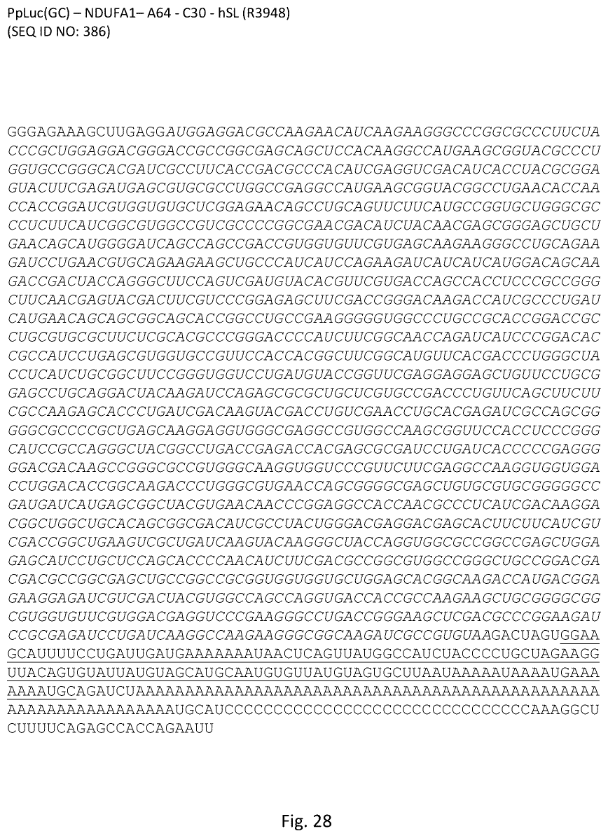

FIG. 28: shows SEQ ID NO. 386, i.e. the mRNA sequence of PpLuc(GC)-NDFUA1-A64-C30-hSL. (R3948). The PpLuc(GC) ORF is highlighted in italics. The 3′-UTR element, which is derived from human NDUFA1 is underlined.

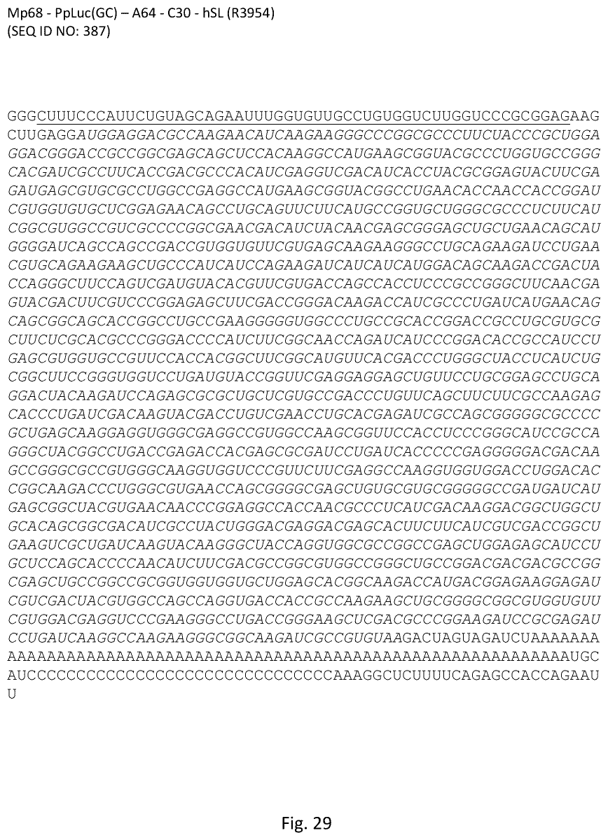

FIG. 29: shows SEQ ID NO. 387, i.e. the mRNA sequence of Mp68-PpLuc(GC)-A64-C30-hSL. (R3954). The PpLuc(GC) ORF is highlighted in italics. The 5′-UTR element, which is derived from murine mp68 is underlined.

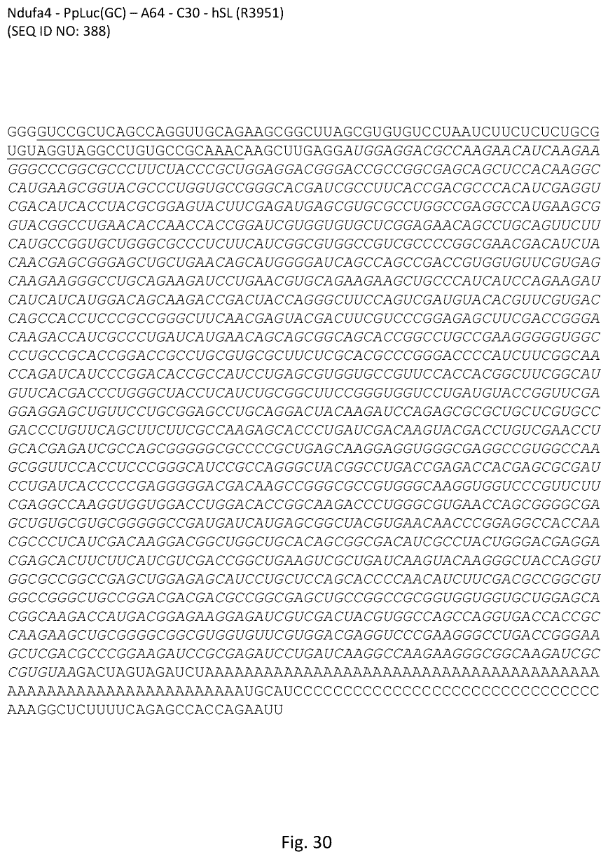

FIG. 30: shows SEQ ID NO. 388, i.e. the mRNA sequence of Ndufa4-PpLuc(GC)-A64-C30-hSL. (R3951). The PpLuc(GC) ORF is highlighted in italics. The 5′-UTR element, which is derived from murine ndufa4 is underlined.

### EXAMPLES

1. Identification of 3′-untranslated region elements (3′-UTR elements) and/or 5′-untranslated region elements (5′-UTR elements) prolonging and/or increasing protein production:

mRNA decay in different human and murine cell types was assessed by pulse-chase methodology. To this end, three different human cell types (HeLa, HDF and U-937) and three different mouse cell types (NIH3T3, JAWSII and L929) were plated over night in their respective medium: HeLa, U-937, L929 in RPMI medium, JAWSII und NIH3T3 in DMEM and HDF in Fibroblast Growth Medium 2. The cells were incubated for 3 h with the respective medium containing 200 μM 4-thiouridine (4sU) for labelling of newly synthesized RNA (“pulse”). After incubation (labelling), cells are washed once and the medium was replaced by fresh medium supplemented with 2 mM Uridine (“chase”). Cells were incubated further for 3 h (1st point in time) or 15 h (2^{nd} point in time) before harvesting.

Accordingly, cells were harvested 3 h (1st point in time) and 15 h (2^{nd} point in time) after end of labelling.

The total RNA was isolated from these cells using RNeasy Mini Kit (Qiagen). HPDP-Biotin (EZ-Link Biotin-HPDP, Thermo Scientific; pyridyldithiol-activated, sulfhydryl-reactive biotinylation reagent that conjugates via a cleavable (reversible) disulfide bond) was then incubated with the total RNA in order to extract the 4-thiouridine (4sU)-labelled RNA. HPDP-Biotin specifically reacts with the reduced thiols (—SH) in the 4-thiouridine (4sU)-labelled RNA to form reversible disulfide bonds. The biotinylated RNA was ultrafiltrated using an Amicon-30 device, incubated with streptavidin-coupled dynabeads (Life Technologies) and recovered from streptavidin by DTT. Subsequently, the RNA was purified using RNeasy Mini Kit. For each cell line 3 independent experiments were performed.

The extracted 4sU-labelled RNA was used in a micro array analysis in order to determine the transcript levels of a large variety of mRNA species (i.e. the amounts of the mRNA species) present at a first point in time (3 h after labelling) and the transcript levels of a large variety of mRNA species (i.e. the amounts of the mRNA species) present at a second point in time (15 h after labelling). Affymetrix Human Gene 1.0 ST and Affymetrix Mouse Gene 1.0 ST micro arrays were used. Affymetrix Human Gene 1.0 ST comprises 36079 mRNA species. Affymetrix Mouse Gene 1.0 ST comprises 26166 mRNA species.

Since these micro arrays provide a whole transcript coverage, i.e. they provide a complete expression profile of mRNA, the ratio of the transcript level of a certain mRNA species at the second point in time to the transcript level of the same mRNA species at the first point in time was accordingly determined for a large number of mRNA species. The ratios thus reflect the x-fold transcript level of the mRNA species (shown as Gene Symbol) at the second point in time as compared to the first point in time.

The results from these experiments are shown in Tables 1-3 below. Each of the Tables 1-3 shows a ranking of the most stable mRNA species, i.e. according to the ratio of the transcript level of this mRNA species at the second point in time to the transcript level of this mRNA species at the first point in time (Table 1: combined analysis of human cell types (HeLa, HDF and U-937); Table 2: combined analysis of mouse cell lines (NIH3T3, JAWSII and L929); Table 3: human cell line HDF (human dermal fibroblasts)). Such mRNA species were considered as “most stable mRNA species”, which show a value for the ratio of the transcript level at the first point in time/the transcript level at the second point in time of at least 0,549943138 (approximately 55%; Table 1), 0,676314425 (approximately 68%, Table 2) or 0,8033973 (approximately 80%, Table 3).

Furthermore, the relationship of the ratio of a certain mRNA species to the average ratio (i.e. the average of the ratios of all mRNA species determined, which is shown in the Tables as “Average of the ratio”) was calculated and given as % of average.

TABLE 1
stable mRNAs resulting from the combined analysis of human cell
types (HeLa, HDF and U-937) with the Affymetrix Human Gene
1.0 ST micro array. 113 mRNA species of the 36079 mRNA
species on the micro array were selected as “most stable”
mRNA species. This corresponds to 0.31% of the mRNA
species present on the micro array.

| | Ratio of the | | |
|---|---|---|---|
| | transcript level at | | |
| | the 2nd time to the | | |
| | transcript level at | Average of | % of |
| Gene symbol | the 1st time | the ratio | average |
| LTA4H | 0.982490359 | 0.258826017 | 379.5948991 |
| SLC38A6 | 0.953694877 | | 368.4694789 |
| DECR1 | 0.927429689 | | 358.3216631 |
| PIGK | 0.875178367 | | 338.1338462 |
| FAM175A | 0.849392515 | | 328.1712266 |
| PHYH | 0.827905031 | | 319.8693239 |
| NT5DC1 | 0.815986179 | | 315.2643572 |
| TBC1D19 | 0.805960687 | | 311.3909086 |
| PIGB | 0.805108608 | | 311.0616997 |
| ALG6 | 0.804875859 | | 310.9717748 |
| CRYZ | 0.797694475 | | 308.1971756 |
| BRP44L | 0.796150905 | | 307.6008021 |
| ACADSB | 0.792385554 | | 306.1460216 |
| SUPT3H | 0.792305264 | | 306.1150005 |
| TMEM14A | 0.792128439 | | 306.0466827 |
| GRAMD1C | 0.78766459 | | 304.3220303 |
| C11orf80 | 0.778391775 | | 300.739386 |
| C9orf46 | 0.776061355 | | 299.8390053 |
| ANXA4 | 0.765663559 | | 295.8217134 |
| RAB7A | 0.757621668 | | 292.7146492 |
| TBCK | 0.753324047 | | 291.0542204 |
| AGA | 0.751782245 | | 290.4585303 |
| IFI6 | 0.742389518 | | 286.829557 |
| C2orf34 | 0.737633511 | | 284.9920263 |
| TPK1 | 0.731359535 | | 282.5680135 |
| ALDH6A1 | 0.731062569 | | 282.4532776 |
| AGTPBP1 | 0.725606511 | | 280.3452757 |
| CCDC53 | 0.725535697 | | 280.3179158 |
| LRRC28 | 0.722761729 | | 279.2461657 |
| MBNL3 | 0.716905277 | | 276.9834674 |
| CCDC109B | 0.713320794 | | 275.5985668 |
| PUS10 | 0.70905743 | | 273.9513739 |
| CCDC104 | 0.706185858 | | 272.8419137 |
| CASP1 | 0.699081435 | | 270.0970494 |
| SNX14 | 0.689529842 | | 266.4066965 |
| SKAP2 | 0.686417578 | | 265.2042424 |
| NDUFB6 | 0.683568924 | | 264.1036366 |
| EFHA1 | 0.680321463 | | 262.8489478 |
| BCKDHB | 0.679714289 | | 262.6143601 |
| BBS2 | 0.677825758 | | 261.8847077 |
| LMBRD1 | 0.676629332 | | 261.4224565 |
| ITGA6 | 0.660264393 | | 255.0996998 |
| HERC5 | 0.654495807 | | 252.8709496 |
| HADHB | 0.651220796 | | 251.6056164 |
| MCCC2 | 0.650460461 | | 251.3118537 |
| CAT | 0.647218183 | | 250.0591672 |
| ANAPC4 | 0.646761056 | | 249.8825517 |
| PCCB | 0.641145931 | | 247.7130926 |
| PHKB | 0.639806797 | | 247.1957046 |
| ABCB7 | 0.639415266 | | 247.0444329 |
| PGCP | 0.636830107 | | 246.0456309 |
| GPD2 | 0.63484437 | | 245.2784217 |
| TMEM38B | 0.634688463 | | 245.2181856 |
| NFU1 | 0.63202654 | | 244.1897253 |
| OMA1 | 0.631592924 | | 244.0221934 |
| LOC128322 | 0.630915328 | | 243.7603974 |
| NUBPL | 0.627949735 | | 242.6146113 |
| LANCL1 | 0.627743069 | | 242.5347636 |
| HHLA3 | 0.62723119 | | 242.3369941 |
| PIR | 0.625871255 | | 241.8115696 |
| ACAA2 | 0.624054189 | | 241.1095284 |
| CTBS | 0.621758355 | | 240.22251 |
| GSTM4 | 0.618559637 | | 238.9866536 |
| ALG8 | 0.617468882 | | 238.5652294 |
| ACTR10 | 0.614629804 | | 237.4683237 |
| PIGF | 0.612863425 | | 236.7858655 |
| MGST3 | 0.607459796 | | 234.6981198 |
| SCP2 | 0.604745109 | | 233.6492735 |
| HPRT1 | 0.604586436 | | 233.5879689 |
| ACSF2 | 0.603568827 | | 233.1948052 |
| VPS13A | 0.60079506 | | 232.1231332 |
| CTH | 0.598492068 | | 231.2333494 |
| NXT2 | 0.597938464 | | 231.0194589 |
| MGST2 | 0.596121512 | | 230.3174615 |
| C11orf67 | 0.59596274 | | 230.2561181 |
| PCCA | 0.595915054 | | 230.2376943 |
| GLMN | 0.594596168 | | 229.7281295 |
| DHRS1 | 0.594391166 | | 229.6489249 |
| PON2 | 0.594025719 | | 229.5077308 |
| NME7 | 0.593140523 | | 229.1657265 |
| ETFDH | 0.59290737 | | 229.0756456 |
| ALG13 | 0.591519568 | | 228.5394547 |
| DDX60 | 0.590567649 | | 228.1716714 |
| DYNC2LI1 | 0.590400874 | | 228.1072359 |
| VPS8 | 0.586233686 | | 226.4972016 |
| ITFG1 | 0.585791975 | | 226.3265424 |
| CDK5 | 0.584517109 | | 225.8339853 |
| C1orf112 | 0.58415003 | | 225.6921603 |
| IFT52 | 0.579757269 | | 223.9949738 |
| CLYBL | 0.577777391 | | 223.230028 |
| FAM114A2 | 0.575975081 | | 222.533688 |
| NUDT7 | 0.575398988 | | 222.3111085 |
| AKD1 | 0.57519887 | | 222.233791 |
| MAGED2 | 0.575157132 | | 222.217665 |
| HRSP12 | 0.574805797 | | 222.0819235 |
| STX8 | 0.573508131 | | 221.5805571 |
| ACAT1 | 0.569067306 | | 219.8648003 |
| IFT74 | 0.568627867 | | 219.695019 |
| KIFAP3 | 0.567709483 | | 219.3401921 |
| CAPN1 | 0.567537877 | | 219.2738902 |
| COX11 | 0.566354405 | | 218.8166442 |
| GLT8D4 | 0.566035014 | | 218.6932442 |
| HACL1 | 0.56371793 | | 217.7980159 |
| IFT88 | 0.562663344 | | 217.3905661 |
| NDUFB3 | 0.561240987 | | 216.8410243 |
| ANO10 | 0.561096127 | | 216.7850564 |
| ARL6 | 0.560155258 | | 216.4215424 |
| LPCAT3 | 0.559730076 | | 216.2572689 |
| ABCD3 | 0.55747212 | | 215.3848853 |
| COPG2 | 0.557180095 | | 215.2720583 |
| MIPEP | 0.554396343 | | 214.1965281 |
| LEPR | 0.551799358 | | 213.1931572 |
| C2orf76 | 0.549943138 | | 212.4759882 |

TABLE 2
stable mRNAs resulting from the combined analysis of mouse
cell lines (NIH3T3, JAWSII and L929) with the Affymetrix
Mouse Gene 1.0 ST micro array: 99 mRNA species of the
26166 mRNA species on the micro array were selected as the
“most stable” mRNA species. This corresponds to
0.38% of the mRNA species present on the micro array.

| | Ratio of the | | |
|---|---|---|---|
| | transcript level at | | |
| | the 2nd time to the | | |
| | transcript level at | Average of | |
| ene symbol | the 1st time | the ratio | % of average |
| Ndufa1 | 1.571557917 | 0.209425963 | 750.4121719 |
| Atp5e | 1.444730129 | | 689.8524465 |
| Gstm5 | 1.436992822 | | 686.1579154 |
| Uqcr11 | 1.221605816 | | 583.3115431 |
| Ifi27l2a | 1.203811772 | | 574.8149632 |
| Cbr2 | 1.162403907 | | 555.0428852 |
| Anapc13 | 1.153679871 | | 550.8771953 |
| Atp5l | 1.126858713 | | 538.0702074 |
| Tmsb10 | 1.048459674 | | 500.6350022 |
| Nenf | 1.045891853 | | 499.4088786 |
| Ndufa7 | 1.03898238 | | 496.1096349 |
| Atp5k | 1.03623698 | | 494.7987179 |
| 1110008P14Rik | 1.029513775 | | 491.5884162 |
| Cox4i1 | 0.991815573 | | 473.5876865 |
| Cox6a1 | 0.991620272 | | 473.4944312 |
| Ndufs6 | 0.989419978 | | 472.4438002 |
| Sec61b | 0.984420709 | | 470.0566705 |
| Romo1 | 0.981642576 | | 468.7301241 |
| Gnas | 0.969128675 | | 462.7547898 |
| Snrpd2 | 0.962862199 | | 459.7625743 |
| Mgst3 | 0.96060161 | | 458.6831531 |
| Aldh2 | 0.949761281 | | 453.5069425 |
| 2010107E04Rik | 0.933570825 | | 445.776069 |
| Ssr4 | 0.930263069 | | 444.1966294 |
| Myl6 | 0.920572238 | | 439.5692993 |
| Prdx4 | 0.914830854 | | 436.8278128 |
| Ubl5 | 0.902505176 | | 430.9423544 |
| 1110001J03Rik | 0.888041155 | | 424.0358468 |
| Ndufa13 | 0.881735594 | | 421.0249684 |
| Ndufa3 | 0.880861551 | | 420.6076163 |
| Gstp2 | 0.87970004 | | 420.0529997 |
| Tmem160 | 0.878001416 | | 419.2419142 |
| Ergic3 | 0.87481135 | | 417.7186716 |
| Pgcp | 0.870441149 | | 415.6319192 |
| Slpi | 0.868909664 | | 414.9006418 |
| Myeov2 | 0.868175997 | | 414.5503186 |
| Ndufa4 | 0.862009116 | | 411.6056594 |
| Ndufs5 | 0.857586364 | | 409.4938143 |
| Gstm1 | 0.856672742 | | 409.0575637 |
| 1810027O10Rik | 0.855929863 | | 408.7028424 |
| Atp5o | 0.848957424 | | 405.3735324 |
| Shfm1 | 0.841951399 | | 402.0281856 |
| Tspo | 0.840567742 | | 401.3674952 |
| S100a6 | 0.840163495 | | 401.1744691 |
| Taldo1 | 0.8400757 | | 401.1325475 |
| Bloc1s1 | 0.838838894 | | 400.541978 |
| Hexa | 0.826597959 | | 394.6969835 |
| Ndufb11 | 0.821601877 | | 392.311376 |
| Map1lc3a | 0.816696063 | | 389.968871 |
| Morn2 | 0.810862522 | | 387.18338 |
| Gpx4 | 0.808459051 | | 386.0357329 |
| Mif | 0.804105552 | | 383.9569558 |
| Cox6b1 | 0.803409855 | | 383.6247633 |
| 2900010J23Rik | 0.802900813 | | 383.3816981 |
| Sec61g | 0.797138268 | | 380.6301077 |
| 2900010M23Rik | 0.793618387 | | 378.9493795 |
| Anapc5 | 0.793224505 | | 378.7613023 |
| Mars2 | 0.787395376 | | 375.9779182 |
| Phpt1 | 0.785668786 | | 375.153479 |
| Ndufb8 | 0.784300334 | | 374.5000492 |
| Pfdn5 | 0.779021933 | | 371.9796349 |
| Arpc3 | 0.77876305 | | 371.8560197 |
| Ndufb7 | 0.774103875 | | 369.6312833 |
| Atp5h | 0.772255845 | | 368.7488573 |
| Mrpl23 | 0.77034041 | | 367.834245 |
| Tomm6 | 0.75481818 | | 360.4224467 |
| Mtch1 | 0.752594518 | | 359.3606576 |
| Pcbd2 | 0.752256847 | | 359.199421 |
| Ecm1 | 0.752254099 | | 359.1981094 |
| Hrsp12 | 0.749135357 | | 357.708923 |
| Mecr | 0.746269148 | | 356.3403207 |
| Uqcrq | 0.734462177 | | 350.7025426 |
| Gstm3 | 0.733839044 | | 350.4049993 |
| Lsm4 | 0.732100345 | | 349.5747779 |
| Park7 | 0.7307842 | | 348.9463242 |
| Usmg5 | 0.724562823 | | 345.9756436 |
| Cox8a | 0.720194618 | | 343.8898445 |
| Ly6c1 | 0.716087602 | | 341.9287619 |
| Cox7b | 0.713519017 | | 340.7022736 |
| Ppib | 0.706106711 | | 337.1629288 |
| Bag1 | 0.70488561 | | 336.5798584 |
| S100a4 | 0.701675201 | | 335.046902 |
| Bcap31 | 0.700846929 | | 334.6514056 |
| Tecr | 0.699592215 | | 334.0522852 |
| Rabac1 | 0.699161282 | | 333.8465165 |
| Robld3 | 0.694068018 | | 331.4145049 |
| Sod1 | 0.691852987 | | 330.356837 |
| Nedd8 | 0.691415017 | | 330.1477083 |
| Higd2a | 0.689498548 | | 329.2326025 |
| Trappc6a | 0.688046277 | | 328.5391491 |
| Ldhb | 0.686084572 | | 327.6024437 |
| Nme2 | 0.685974394 | | 327.5498339 |
| Snrpg | 0.684247073 | | 326.7250454 |
| Ndufa2 | 0.683350661 | | 326.2970129 |
| Serf1 | 0.681148053 | | 325.2452768 |
| Oaz1 | 0.681139695 | | 325.2412861 |
| Ybx1 | 0.678927132 | | 324.1847964 |
| Sepp1 | 0.677551422 | | 323.5279009 |
| Gaa | 0.676314425 | | 322.9372402 |

TABLE 3
stable mRNAs resulting from the analysis of the human cell line
HDF (human dermal fibroblasts) with the Affymetrix Human
Gene 1.0 ST micro array: 46 mRNA species of the 36079
mRNA species on the micro array were selected as the “most
stable” mRNA species. This corresponds to 0.13% of
the mRNA species present on the micro array.

| | Ratio of the | | |
|---|---|---|---|
| | transcript level at | | |
| | the 2nd time to | | |
| | thetranscript level at | Average of | |
| Gene symbol | the 1st time | the ratio | % of average |
| ABCA6 | 2.062835692 | 0.278262352 | 741.3276273 |
| LY96 | 1.719983635 | | 618.1158256 |
| CROT | 1.422424006 | | 511.1809038 |
| ENPP5 | 1.315849211 | | 472.880791 |
| SERPINB7 | 1.12288882 | | 403.5360196 |
| TCP11L2 | 1.103519648 | | 396.5752607 |
| IRAK1BP1 | 1.05490107 | | 379.1030521 |
| CDKL2 | 1.042002646 | | 374.4677057 |
| GHR | 1.039327135 | | 373.5061992 |
| KIAA1107 | 1.020519239 | | 366.7471477 |
| RPS6KA6 | 1.017695602 | | 365.7324085 |
| CLGN | 1.007943464 | | 362.2277524 |
| TMEM45A | 1.006063873 | | 361.5522781 |
| TBC1D8B | 0.979626826 | | 352.0515148 |
| ACP6 | 0.964241225 | | 346.5223439 |
| RP6-213H19.1 | 0.960702414 | | 345.2505905 |
| C11orf74 | 0.960086216 | | 345.0291458 |
| SNRPN | 0.939315038 | | 337.5645433 |
| GLRB | 0.923441342 | | 331.8599644 |
| HERC6 | 0.919865006 | | 330.5747254 |
| CFH | 0.908835974 | | 326.6111879 |
| GALC | 0.90862766 | | 326.5363257 |
| PDE1A | 0.908445187 | | 326.4707497 |
| GSTM5 | 0.902862912 | | 324.4646303 |
| CADPS2 | 0.89753131 | | 322.5485959 |
| AASS | 0.894768872 | | 321.5558503 |
| TRIM6-TRIM34 | 0.892150571 | | 320.6149031 |
| SEPP1 | 0.891344657 | | 320.3252795 |
| PDE5A | 0.890221551 | | 319.9216656 |
| SATB1 | 0.885139895 | | 318.0954552 |
| CCPG1 | 0.88148167 | | 316.7807873 |
| CNTN1 | 0.87246423 | | 313.5401621 |
| LMBRD2 | 0.871500964 | | 313.1939903 |
| TLR3 | 0.86777981 | | 311.8567077 |
| BCAT1 | 0.864255836 | | 310.5902863 |
| TOM1L1 | 0.86240499 | | 309.925142 |
| SLC35A1 | 0.857201353 | | 308.055095 |
| GLYATL2 | 0.85132258 | | 305.9424223 |
| STAT4 | 0.840572034 | | 302.0789653 |
| GULP1 | 0.839518351 | | 301.7003001 |
| EHHADH | 0.82971807 | | 298.1783427 |
| NBEAL1 | 0.82554089 | | 296.6771768 |
| KIAA1598 | 0.820341324 | | 294.8085928 |
| HFE | 0.815037603 | | 292.9025779 |
| KIAA1324L | 0.808279102 | | 290.4737547 |
| MANSC1 | 0.8033973 | | 288.7193664 |

2. Cloning of 5′- and 3′-UTR Elements of Stably Expressed mRNAs:

The nucleotide sequence of the 5′- and/or 3′-UTRs of the mRNA species shown in Table 1-3 were determined by data base search and amplified by PCR or synthesized by oligo annealing. The resulting PCR fragments were cloned into a vector as described in detail in Example 3 below. 5′-UTR elements were cloned into the vector PpLuc(GC)-albumin7-A64-C30-hSL (SEQ ID NO. 41, FIG. 7); and 3′-UTR elements were cloned into the vector 32L4-PpLuc(GC)-A64-C30-hSL (SEQ ID NO. 35, FIG. 1) or into the vector PpLuc(GC)-A64-C30-hSL (SEQ ID NO. 44, FIG. 10).

### 3. Preparation of DNA-Templates

A vector for in vitro transcription was constructed containing a T7 promoter and a GC-enriched sequence coding for Photinus pyralis luciferase (PpLuc(GC)). An A64 poly(A) sequence, followed by C30 and a histone stem-loop sequence, was inserted 3′ of PpLuc(GC). The histone stem-loop sequence was followed by a restriction site used for linearization of the vector before in vitro transcription.

To investigate the effect of different 3′-UTR elements on protein expression, a vector as described above was used (control) and this vector was modified to include a 3′-UTR element of interest. Alternatively, a vector was constructed as described above, whereby the 5′ untranslated region (5′-UTR) of 32L4 (ribosomal protein Large 32) was inserted 5′ of PpLuc(GC). This vector was then modified to include either different 3′-UTR elements or no 3′-UTR (control).

Particularly, the following mRNAs were obtained from these vectors accordingly by in vitro transcription (the mRNA sequences are depicted in FIGS. 1 to 6, FIGS. 10, 11 and FIGS. 19 to 21):
32L4-PpLuc(GC)-A64-C30-hSL (SEQ ID NO. 35, FIG. 1);
32L4-PpLuc(GC)-gnas-A64-C30-hSL (SEQ ID NO. 36, FIG. 2);
32L4-PpLuc(GC)-morn2-A64-C30-hSL (SEQ ID NO. 37, FIG. 3);
32L4-PpLuc(GC)-gstm1-A64-C30-hSL (SEQ ID NO. 38, FIG. 4);
32L4-PpLuc(GC)-ndufa1-A64-C30-hSL (SEQ ID NO. 39, FIG. 5);
32L4-PpLuc(GC)-cbr2-A64-C30-hSL (SEQ ID NO. 40, FIG. 6);
PpLuc(GC)-A64-C30-hSL (SEQ ID NO. 44, FIG. 10);
PpLuc(GC)-gnas-A64-C30-hSL (SEQ ID NO. 45, FIG. 11);
32L4-PpLuc(GC)-Ybx1(V2)-A64-C30-hSL (SEQ ID NO. 46, FIG. 19);
32L4-PpLuc(GC)-Ndufb8-A64-C30-hSL (SEQ ID NO. 47, FIG. 20); and
32L4-PpLuc(GC)-Cntn1-004(V2)-A64-C30-hSL (SEQ ID NO. 48, FIG. 21).

An alternative sequence for the construct 32L4-PpLuc(GC)-A64-C30-hSL is shown in FIG. 25 (SEQ ID NO. 383). However, SEQ ID NO. 35, FIG. 1 was used in the Examples as described herein and is, thus, preferred for the construct 32L4-PpLuc(GC)-A64-C30-hSL.

To investigate the effect of different 5′-UTR elements on protein expression, a vector was constructed as described above, whereby the 3′ untranslated region (3′-UTR) of albumin7 (3′-UTR of human albumin with three single point mutations introduced to remove a T7 termination signal as well as a HindIII and XbaI restriction site) was inserted 3′ of PpLuc(GC). This vector was modified to include either different 5′-UTR elements or no 5′-UTR (control).

Particularly, the following mRNAs were obtained from these vectors accordingly by in vitro transcription (the mRNA sequences are depicted in FIGS. 7 to 9):
PpLuc(GC)-albumin7-A64-C30-hSL (SEQ ID NO. 41, FIG. 7);
Mp68-PpLuc(GC)-albumin7-A64-C30-hSL (SEQ ID NO. 42, FIG. 8); and
Ndufa4-PpLuc(GC)-albumin7-A64-C30-hSL (SEQ ID NO. 43, FIG. 9);

### 4. In Vitro Transcription

The DNA templates according to Example 2 and 3 were linearized and transcribed in vitro using T7-RNA polymerase. The DNA templates were then digested by DNase-treatment. mRNA transcripts contained a 5′-CAP structure obtained by adding an excess of N7-Methyl-Guanosine-5′-Triphosphate-5′-Guanosine to the transcription reaction. mRNA thus obtained was purified and resuspended in water.

5. Luciferase Expression by mRNA Lipofection

Human dermal fibroblasts (HDF) and HeLa cells were seeded in 96 well plates at a density of 1×10⁴ cells per well. The following day, cells were washed in Opti-MEM and then transfected with 12.5 ng per well of Lipofectamine2000-complexed PpLuc-encoding mRNA in Opti-MEM. Untransfected cells served as control. mRNA coding for Renilla reniformis luciferase (RrLuc) was transfected together with PpLuc mRNA to control for transfection efficiency (1 ng of RrLuc mRNA per well). 90 minutes after start of transfection, Opti-MEM was exchanged for medium. 6, 24, 48, 72 hours after transfection, medium was aspirated and cells were lysed in 100 l of Passive Lysis buffer (Promega). Lysates were stored at −80° C. until luciferase activity was measured.

### 6. Luciferase Measurement

Luciferase activity was measured as relative light units (RLU) in a Hidex Chameleon plate reader. The activities of Ppluc and Rrluc are measured sequentially from a single sample in a dual luciferase assay. The PpLuc activity was measured first at 2 seconds measuring time using 20 l of lysate and 50 l of Beetle juice (pjk GmbH). After 1500 ms delay RrLuc activity is measured with 50 μl Renilla juice (pjk GmbH).

### 7. Results

a. Protein Expression from mRNA Containing 3′-UTR Elements According to the Invention is Increased and/or Prolonged.

To investigate the effect of various 3′-UTR elements on protein expression from mRNA, mRNAs containing different 3′-UTR elements were compared to an mRNA lacking a 3′-UTR.

Human HeLa and HDF cells were transfected with Luciferase encoding mRNAs and Luciferase levels (in RLU) were measured 6, 24, 48, and 72 hours after transfection. The PpLuc signal was corrected for transfection efficiency by the signal of cotransfected RrLuc. Normalized PpLuc levels at 24 h were set to 100% and relative expression to 24 h is calculated (see following Table 4 and FIGS. 12 (HeLa cells) and 13 (HDF cells)).

TABLE 4

| | HeLa | | | HDF | | |
|---|---|---|---|---|---|---|
| mRNA | 24 h | 48 h | 72 h | 24 h | 48 h | 72 h |
| 32L4-PpLuc(GC)-A64-C30-hSL | 100 | 12.3 | 2.7 | 100 | 34.8 | 10.9 |
| 32L4-PpLuc(GC)-gnas-A64-C30-hSL | 100 | 50.5 | 30.9 | 100 | 79.8 | 27.8 |
| 32L4-PpLuc(GC)-morn2-A64-C30-hSL | 100 | 32.9 | 10.5 | 100 | 44.5 | 14.6 |
| 32L4-PpLuc(GC)-gstm1-A64-C30-hSL | 100 | 24.8 | 7.6 | 100 | 46.5 | 21.4 |
| 32L4-PpLuc(GC)-ndufa1-A64-C30-hSL | 100 | 29.4 | 10.6 | 100 | 41.9 | 13.9 |
| 32L4-PpLuc(GC)-cbr2-A64-C30-hSL | 100 | 21.9 | 4.9 | 100 | 60.0 | 23.2 |

Table 4 shows relative PpLuc expression normalized to RrLuc (mean values of three independent experiments are given).

Luciferase was expressed from mRNA lacking a 3′-UTR. However, the inventive 3′-UTR elements gnas, morn2, gstm1, ndufa and cbr2 significantly prolonged luciferase expression.

b. Protein Expression from mRNA Containing 5′-UTR Elements According to the Invention is Increased and/or Prolonged.

To investigate the effect of various 5′-UTR elements on protein expression from mRNA, mRNAs containing different 5′-UTRs were compared to an mRNA lacking a 5′-UTR.

Human HeLa and HDF cells were transfected with Luciferase encoding mRNAs and Luciferase levels were measured 6, 24, 48, and 72 hours after transfection. The PpLuc signal was corrected for transfection efficiency by the signal of cotransfected RrLuc. Total protein expression from 0 to 72 hours was calculated as the area under the curve (AUC). The levels of the control construct without 5′ UTR was set to 1 (see following Table 5 and FIG. 14 (HeLa cells) and 15 (HDF cells)).

TABLE 5

| mRNA | AUC HeLa | AUC HDF |
|---|---|---|
| PpLuc(GC)-albumin7-A64-C30-hSL | 1.00 | 1.07 |
| Mp68-PpLuc(GC)-albumin7-A64-C30-hSL | 1.79 | 3.03 |
| Ndufa4-PpLuc(GC)-albumin7-A64-C30-hSL | 1.92 | 2.83 |

Table 5 shows the total PpLuc expression normalized to RrLuc (mean values of three independent experiments are given).

Luciferase was expressed from mRNA lacking a 5′-UTR. However, the inventive 5′-UTR elements mp68 and ndufa4 significantly increased luciferase expression.

c. Protein Expression from mRNA Containing 3′-UTR Elements According to the Invention is Prolonged.

To investigate the effect of various 3′UTRs on protein expression from mRNA, mRNAs containing different 3′UTRs were compared to an mRNA lacking a 3′UTR.

Human HeLa and HDF cells were transfected with Luciferase encoding mRNAs and Luciferase levels (in RLU) were measured 24, 48, and 72 hours after transfection. The PpLuc signal was corrected for transfection efficiency by the signal of cotransfected RrLuc. Normalized PpLuc levels at 24 h were set to 100% and relative expression to 24 h is calculated (see following Table 6 and FIGS. 16 (HeLa cells) and 17 (HDF cells)).

TABLE 6

| | HeLa | | | HDF | | |
|---|---|---|---|---|---|---|
| mRNA | 24 h | 48 h | 72 h | 24 h | 48 h | 72 h |
| PpLuc(GC)-gnas-A64-C30-hSL | 100 | 61.1 | 30.3 | 100 | 53.6 | 34.2 |
| PpLuc(GC)-A64-C30-hSL | 100 | 17.1 | 2.7 | 100 | 29.0 | 12.4 |

Table 6 shows relative PpLuc expression normalized to RrLuc (mean values of three independent experiments are given).

d. Protein Expression from mRNA Containing 3′-UTR Elements According to the Invention is Prolonged.

To investigate the effect of various 3′UTRs on protein expression from mRNA, mRNAs containing different 3′UTRs were compared to an mRNA lacking a 3′UTR.

Human HeLa and HDF cells were transfected with Luciferase encoding mRNAs and Luciferase levels were measured 6, 24, 48, and 72 hours after transfection. The PpLuc signal was corrected for transfection efficiency by the signal of cotransfected RrLuc. Total protein expression from 0 to 72 hours was calculated as the area under the curve (AUC). The levels of the control construct without 5′ UTR was set to 1 (see following Table 7 and FIG. 18 (HDF cells) and 17 (HeLa cells)).

Human HeLa and HDF cells were transfected with Luciferase encoding mRNAs and Luciferase levels (in RLU) were measured 24, 48, and 72 hours after transfection. The PpLuc signal was corrected for transfection efficiency by the signal of cotransfected RrLuc. Normalized PpLuc levels at 24 h were set to 100% and relative expression to 24 h is calculated (see following Table 7 and FIG. 18 (HDF cells)).

TABLE 7

| | HDF | | |
|---|---|---|---|
| mRNA | 24 h | 48 h | 72 h |
| 32L4-PpLuc(GC)-Ybx1-001(V2)-A64-C30-hSL | 100 | 57.0 | 28.5 |
| 32L4-PpLuc(GC)-Ndufb8-A64-C30-hSL | 100 | 65.4 | 37.6 |
| 32L4-PpLuc(GC)-Cntn1004(V2)-A64-C30-hSL | 100 | 71.0 | 47.7 |
| 32L4-PpLuc(GC)-A64-C30-hSL | 100 | 45.2 | 21.87 |

Table 7 shows relative PpLuc expression normalized to RrLuc (mean values of three independent experiments are given).

### 8. Effect of Further 3′UTRs on Protein Expression

To further investigate the effect of various 3′UTRs on protein expression from mRNA, new mRNA constructs were prepared and those mRNAs containing different 3′UTRs were compared to an mRNA lacking a 3′UTR.

To this end, selected 3′-UTR elements (gnas, morn2, ndufa1 and NDUFA1) were cloned into the vector PpLuc(GC)-A64-C30-hSL (SEQ ID NO. 44, FIG. 10), which was constructed containing a T7 promoter and a GC-enriched sequence coding for Photinus pyralis luciferase (PpLuc(GC)). An A64 poly(A) sequence, followed by C30 and a histone stem-loop sequence, was inserted 3′ of PpLuc(GC). The histone stem-loop sequence was followed by a restriction site used for linearization of the vector before in vitro transcription.

In particular, the following mRNAs were obtained from such vectors by in vitro transcription (the mRNA sequences are depicted in FIGS. 11 and 26 to 28:
PpLuc(GC)-gnas-A64-C30-hSL (SEQ ID NO. 45, FIG. 11);
PpLuc(GC)-morn2-A64-C30-hSL (SEQ ID NO. 384, FIG. 26);
PpLuc(GC)-ndufa1-A64-C30-hSL (SEQ ID NO. 385, FIG. 27); and
PpLuc(GC)-NDUFA1-A64-C30-hSL (SEQ ID NO. 386, FIG. 28).

Human HeLa cells were transfected with Luciferase encoding mRNAs and Luciferase levels were measured 24, 48, and 72 hours after transfection. The PpLuc signal was corrected for transfection efficiency by the signal of cotransfected RrLuc (see following Table 8 and FIG. 22).

TABLE 8
relative PpLuc expression normalized to RrLuc (mean
values of 3 independent experiments are given).

| | HeLa (expression in %) | | |
|---|---|---|---|
| mRNA | 24 h | 48 h | 72 h |
| PpLuc(GC)-gnas-A64-C30-hSL | 100 | 77.9 | 36.7 |
| PpLuc(GC)-morn2-A64-C30-hSL | 100 | 53.8 | 17.2 |
| PpLuc(GC)-ndufa1-A64-C30-hSL | 100 | 55.2 | 17.9 |
| PpLuc(GC)-NDUFA1-A64-C30-hSL | 100 | 66.9 | 29.4 |
| PpLuc(GC)-A64-C30-hSL | 100 | 41.5 | 9.6 |

These data and the data shown in FIG. 22 show that protein expression from mRNA containing 3′-UTR elements according to the invention is prolonged.

### 9. Effect of Further 5′UTRs on Protein Expression

To further investigate the effect of various 5′UTRs on protein expression from mRNA, new mRNA constructs were prepared and those mRNAs containing different 5′UTRs were compared to an mRNA lacking a 5′UTR.

To this end, selected 5′-UTR elements (mp68 and ndufa4) were cloned into the vector PpLuc(GC)-A64-C30-hSL (SEQ ID NO. 44, FIG. 10), which was constructed containing a T7 promoter and a GC-enriched sequence coding for Photinus pyralis luciferase (PpLuc(GC)). An A64 poly(A) sequence, followed by C30 and a histone stem-loop sequence, was inserted 3′ of PpLuc(GC). The histone stem-loop sequence was followed by a restriction site used for linearization of the vector before in vitro transcription.

In particular, the following mRNAs were obtained from such vectors by in vitro transcription (the mRNA sequences are depicted in FIGS. 29 and 30:
Mp68-PpLuc(GC)-A64-C30-hSL (SEQ ID NO. 387, FIG. 29); and
Ndufa4-PpLuc(GC)-A64-C30-hSL (SEQ ID NO. 388, FIG. 30).

Human HDF and HeLa cells were transfected with Luciferase encoding mRNAs and Luciferase levels were measured 24, 48, and 72 hours after transfection. The PpLuc signal was corrected for transfection efficiency by the signal of cotransfected RrLuc. Total protein expression (area under the curve) was calculated. The levels of the control construct without 5′ UTR was set to 1 (see following Table 9 and FIGS. 23 and 24).

TABLE 9
total PpLuc expression normalized to RrLuc
(mean RLU values are given).

| mRNA | AUC HDF | AUC HeLa |
|---|---|---|
| PpLuc(GC)-A64-C30-hSL | 1.0 | 1.0 |
| Mp68-PpLuc(GC)-A64-C30-hSL | 3.9 | 2.3 |
| Ndufa4-PpLuc(GC)-A64-C30-hSL | 4.0 | 2.0 |

These data and the data shown in FIGS. 23 and 24 show that protein expression from mRNA containing 5′-UTR elements according to the invention is increased.

1. An artificial nucleic acid molecule comprising:
(a) at least one open reading frame (ORF); and
(b) at least one heterologous 3′-untranslated region element (3′-UTR element),
that comprises at least 50 consecutive nucleotides of the RNA sequence of SEQ ID NO: 2.

2. (canceled)

3. The artificial nucleic acid molecule according to claim 1 comprising at least one 5′-UTR element.

4. The artificial nucleic acid molecule according to claim 3, wherein each of the at least one open reading frame, the at least one 3′-UTR element and the at least one 5′-UTR element are heterologous to each other.

5-11. (canceled)

12. The artificial nucleic acid molecule according to claim 3, wherein:
(i) the at least one 5′-UTR element is derived from a human or a murine gene selected from the group consisting of: housekeeping genes, genes coding for a membrane protein, genes involved in cellular metabolism, genes involved in transcription, translation and replication processes, genes involved in protein modification and genes involved in cell division; or
(ii) the 5′-UTR is not a 5′ TOP UTR.

13-24. (canceled)

25. The artificial nucleic acid molecule according to claim 1, wherein the at least one 3′-UTR element comprises or consists of a nucleic acid sequence which has an identity of at least about to a nucleic acid sequence selected from the group consisting of SEQ ID NOs: 1 to 4 and SEQ ID NO: 116.

26. The artificial nucleic acid molecule according claim 1, wherein the at least one 5′-UTR element comprises or consists of a nucleic acid sequence which has an identity of at least about 80% to a nucleic acid sequence selected from the group consisting of SEQ ID NOs: 25 to 30 and SEQ ID NOs: 319 to 38.

27. (canceled)

28. The artificial nucleic acid molecule according to claim 1, wherein the at least one 3′-UTR element and/or at least one 5′-UTR element exhibits a length of between 3 and about 500 nucleotides.

29. The artificial nucleic acid molecule according to claim 1 further comprising
c. a poly(A) sequence and/or a polyadenylation signal.

30-33. (canceled)

34. The artificial nucleic acid molecule according to claim 1, further comprising a 5′-cap structure, a poly(C) sequence, a histone stem-loop, and/or an IRES-motif.

35. The artificial nucleic acid molecule according to claim 34, wherein the histone stem-loop comprises a sequence according to SEQ ID NO: 34.

36. The artificial nucleic acid molecule according to claim 1, wherein the nucleic acid comprises a promoter.

37. The artificial nucleic acid molecule according to claim 1, wherein the nucleic acid comprises a 5′-TOP UTR.

38-41. (canceled)

42. A vector comprising an artificial nucleic acid molecule according to claim 1.

43-46. (canceled)

47. A cell comprising the artificial nucleic acid molecule according to claim 1.

48-49. (canceled)

50. A pharmaceutical composition comprising the artificial nucleic acid molecule according to claim 1.

51-53. (canceled)

54. A method for treating or preventing a disorder comprising administering the artificial nucleic acid molecule according to claim 1 to a patient in need thereof.

55. A method of treating or preventing a disorder comprising transfection of a cell with an artificial nucleic acid molecule according to claim 1.

56-80. (canceled)

81. The artificial nucleic acid molecule according to claim 3, wherein the at least one 5′-UTR element is derived from a 5′-TOP UTR.

82. The artificial nucleic acid molecule according to claim 81, wherein the at least one 5′-UTR element comprises a sequence of SEQ ID NO: 33.
